SEQUENCE LISTING (466 sequences)

SEQ ID NO: 1 - DNA, 29902 bp, betacoronavirus SARS coronavirus 2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggccgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - protein, 7096 aa, betacoronavirus SARS coronavirus 2 [sequence omitted]

SEQ ID NO: 3 - protein, 1273 aa, betacoronavirus SARS coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 220 aa, Artificial Sequence
  Synthetic sequence surface glycoprotein RBD [SARS-CoV-2 Wuhan seafood market pneumonia virus]; GenBank QHD43416.1; January 23, 2020
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTG

SEQ ID NO: 5 - protein, 72 aa, Artificial Sequence
  Receptor Binding Motif (RBM) in surface glycoprotein RBD [SARS-CoV-2 Wuhan seafood market pneumonia virus]; GenBank QHD43416.1; January 23, 2020
NSNNLDSKVGGNYNYLYRLFRKSNLKPFERDISTEIYQAGSTPCNGVEGFNCYFPLQSYGFQPTNGVGYQPY

SEQ ID NO: 6 - protein, 330 aa, Artificial Sequence
  Synthetic sequence SARS-CoV-2 CH1-CH3 LS G1m17 IgHG1*01
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 7 - protein, 330 aa, Artificial Sequence
  Synthetic sequence SARS-CoV-2 CH1-CH3 LS, ALE G1m17 IgHG1*01
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 8 - protein, 106 aa, Artificial Sequence
  Synthetic sequence SARS-CoV-2 CL IgLC*01
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 107 aa, Artificial Sequence [sequence omitted]

SEQ ID NO: 10 - protein, 18 aa, Artificial Sequence
  Synthetic linker sequence
GSTSGSGKPGSGEGSTKG

SEQ ID NO: 11 - protein, 10 aa, Artificial Sequence
  Synthetic linker sequence
GSGKPGSGEG

SEQ ID NO: 12 - protein, 8 aa, Artificial Sequence
  Synthetic linker sequence
GKPGSGEG

SEQ ID NO: 13 - protein, 8 aa, Artificial Sequence
  Synthetic linker sequence
SGKPGSGE

SEQ ID NO: 14 - protein, 6 aa, Artificial Sequence
  Synthetic linker sequence
  VARIANT (1)..(1): Xaa = positively charged amino acid
  VARIANT (3)..(5): Xaa = glycine (G) or serine (S)
  VARIANT (6)..(6): Xaa = glycine (G) or a negatively charged amino acid
XPXXXX

SEQ ID NO: 15 - protein, 110 aa, Artificial Sequence
  Synthetic linker sequence
  VARIANT (2)..(10): Any one or all of amino acids 2-10 can either be present or absent
  VARIANT (12)..(110): any one or all of amino acids 12-110 can either be present or absent
GGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGS

SEQ ID NO: 16 - protein, 15 aa, Artificial Sequence
  Synthetic linker sequence
GGGGSGGGGSGGGGS

SEQ ID NO: 17 - protein, 50 aa, Artificial Sequence
  Synthetic linker sequence
GGGGSGGGGSGGGGSGGGGSGGGGSGGGGSGGGGSGGGGSGGGGSGGGGS

SEQ ID NO: 18 - protein, 18 aa, Artificial Sequence
  Synthetic linker sequence
GSTSGGGSGGGSGGGGSS

SEQ ID NO: 19 - protein, 14 aa, Artificial Sequence
  Synthetic linker sequence
EGKSSGSGSESKVD

SEQ ID NO: 20 - protein, 18 aa, Artificial Sequence
  Synthetic linker sequence
KESGSVSSEQLAQFRSLD

SEQ ID NO: 21 - protein, 5 aa, Artificial Sequence
  Synthetic linker sequence
GGGGS

SEQ ID NO: 22 - protein, 120 aa, Artificial Sequence
  Synthetic sequence SARS-CoV-2 S2H7-v1 mAb VH
EVQLVESGGGLVQPGGSLRLSCAASGFIVSSNYMSWVRQAPGKGLEWVSVIYSGGSTYYADSVKGRFTISRDNSKNTLYLQMNSLRAEDTSVYYCARDRQYSGSPSFDYWGQGTLVTVSS

SEQ ID NO: 23 - protein, 8 aa, Artificial Sequence
  Synthetic sequence SARS-CoV-2 S2H7-v1 mAb CDRH1
GFIVSSNY

SEQ ID NO: 24 - protein, 7 aa, Artificial Sequence
  Synthetic sequence SARS-CoV-2 S2H7-v1 mAb CDRH2
IYSGGST

SEQ ID NO: 25 - protein, 14 aa, Artificial Sequence
  Synthetic sequence SARS-CoV-2 S2H7-v1 mAb CDRH3
ARDRQYSGSPSFDY

SEQ ID NO: 26 - protein, 108 aa, Artificial Sequence
  Synthetic sequence SARS-CoV-2 S2H7-v1 mAb VL(VK)
DIQMTQSPSSLSASVGDRVTITCRASQSISSYLNWYQQKPGKAPKLLIYTASSLQSGVPSRFSGSGSGTDFTLTISSLQPEDFATYYCQQSYSTPGLTFGGGTKVEIK

SEQ ID NO: 27 - protein, 6 aa, Artificial Sequence
  Synthetic sequence SARS-CoV-2 S2H7-v1 mAb CDRL1
QSISSY

SEQ ID NO: 28 - protein, 3 aa, Artificial Sequence
  Synthetic sequence SARS-CoV-2 S2H7-v1 mAb CDRL2
TAS

SEQ ID NO: 29 - protein, 10 aa, Artificial Sequence
  Synthetic sequence SARS-CoV-2 S2H7-v1 mAb CDRL3
QQSYSTPGLT

SEQ ID NO: 30 - protein, 120 aa, Artificial Sequence
  Synthetic sequence SARS-CoV-2 S2H7-v2 mAb VH
EVQLVESGGGLVQPGGSLRLSCAASGFIVSSNYMSWVRQAPGKGLEWVSVIYSGGSTYYAESVKGRFTISRDNSKNTLYLQMNSLRAEDTSVYYCARDRQYSGSPSFDYWGQGTLVTVSS

SEQ ID NO: 31 - protein, 16 aa, Artificial Sequence
  Synthetic sequence SARS-CoV-2 S2H7-v2 mAb CDRH2
VIYSGGSTYYAESVKG

SEQ ID NO: 32 - protein, 120 aa, Artificial Sequence
  Synthetic sequence SARS-CoV-2 S2H7-v3 mAb VH
EVQLVESGGGLVQPGGSLRLSCAASGFIVSSNYISWVRQAPGKGLEWVSVIYSGGSTYYADSVKGRFTISRDNSKNTLYLQMNSLRAEDTSVYYCARDRQYSGSPSFDYWGQGTLVTVSS

SEQ ID NO: 33 - protein, 10 aa, Artificial Sequence
  Synthetic sequence SARS-CoV-2 S2H7-v3 mAb CDRH1
GFIVSSNYIS

SEQ ID NO: 34 - protein, 120 aa, Artificial Sequence
  Synthetic sequence SARS-CoV-2 S2H7-v4 mAb VH
EVQLVESGGGLVQPGGSLRLSCAASGFIVSSNYISWVRQAPGKGLEWVSVIYSGGSTYYAESVKGRFTISRDNSKNTLYLQMNSLRAEDTSVYYCARDRQYSGSPSFDYWGQGTLVTVSS

SEQ ID NO: 35 - protein, 120 aa, Artificial Sequence
  Synthetic sequence SARS-CoV-2 S2H7-v5 mAb VH
EVQLVESGGGLVQPGGSLRLSCAASGFIVSSNYISWVRQAPGKGLEWVSVIYSGGSTYYADAVKGRFTISRDNSKNTLYLQMNSLRAEDTSVYYCARDRQYSGSPSFDYWGQGTLVTVSS

SEQ ID NO: 36 - protein, 16 aa, Artificial Sequence
  Synthetic sequence SARS-CoV-2 S2H7-v5 mAb CDRH2
VIYSGGSTYYADAVKG

SEQ ID NO: 37 - protein, 120 aa, Artificial Sequence
  Synthetic sequence SARS-CoV-2 S2H13-v1 mAb VH
EVQLVESGGDSVQPGGSLRLSCAAAGFTFSSYWMNWVRQAPGKGLEWVANIKQDGSEKYYVDSVKGRFTISRDNAKNSLYLQMNSLRAEDTAVYYCALSSGYSGYAGNYWGQGTLVTVSS

SEQ ID NO: 38 - protein, 8 aa, Artificial Sequence
  Synthetic sequence SARS-CoV-2 S2H13-v1 mAb CDRH1
GFTFSSYW

SEQ ID NO: 39 - protein, 8 aa, Artificial Sequence
  Synthetic sequence SARS-CoV-2 S2H13-v1 mAb CDRH2
IKQDGSEK

SEQ ID NO: 40 - protein, 13 aa, Artificial Sequence
  Synthetic sequence SARS-CoV-2 S2H13-v1 mAb CDRH3
ALSSGYSGYAGNY

SEQ ID NO: 41 - protein, 110 aa, Artificial Sequence
  Synthetic sequence SARS-CoV-2 S2H13-v1 mAb VL
QAVVTQEPSLTVSPGGTVTLTCGSSTGAVTSGHYPYWFQQKPGQAPRTLIYDTSNKHSWTPARFSGSLLGGKAALTLSGARPEDEAEYYCLLSYSGARGVFGGGTKLTVL

SEQ ID NO: 42 - protein, 9 aa, Artificial Sequence
  Synthetic sequence SARS-CoV-2 S2H13-v1 mAb CDRL1
TGAVTSGHY

SEQ ID NO: 43 - protein, 3 aa, Artificial Sequence
  Synthetic sequence SARS-CoV-2 S2H13-v1 mAb CDRL2
DTS

SEQ ID NO: 44 - protein, 10 aa, Artificial Sequence
  Synthetic sequence SARS-CoV-2 S2H13-v1 mAb CDRL3
LLSYSGARGV

SEQ ID NO: 45 - protein, 120 aa, Artificial Sequence
  Synthetic sequence SARS-CoV-2 S2H13-v2 mAb VH
EVQLVESGGDSVQPGGSLRLSCAAAGFTFSSYFMNWVRQAPGKGLEWVANIKQDGSEKYYVDSVKGRFTISRDNAKNSLYLQMNSLRAEDTAVYYCALSSGYSGYAGNYWGQGTLVTVSS

SEQ ID NO: 46 - protein, 10 aa, Artificial Sequence
  Synthetic sequence SARS-CoV-2 S2H13-v2 mAb CDRH1
GFTFSSYFMN

SEQ ID NO: 47 - protein, 120 aa, Artificial Sequence
  Synthetic sequence SARS-CoV-2 S2H13-v3 mAb VH
EVQLVESGGDSVQPGGSLRLSCAAAGFTFSSYWMNWVRQAPGKGLEWVANIKQEGSEKYYVDSVKGRFTISRDNAKNSLYLQMNSLRAEDTAVYYCALSSGYSGYAGNYWGQGTLVTVSS

SEQ ID NO: 48 - protein, 17 aa, Artificial Sequence
  Synthetic sequence SARS-CoV-2 S2H13-v3 mAb CDRH2
NIKQEGSEKYYVDSVKG

SEQ ID NO: 49 - protein, 120 aa, Artificial Sequence
  Synthetic sequence SARS-CoV-2 S2H13-v4 mAb VH
EVQLVESGGDSVQPGGSLRLSCAAAGFTFSSYFMNWVRQAPGKGLEWVANIKQEGSEKYYVDSVKGRFTISRDNAKNSLYLQMNSLRAEDTAVYYCALSSGYSGYAGNYWGQGTLVTVSS

SEQ ID NO: 50 - protein, 120 aa, Artificial Sequence
  Synthetic sequence SARS-CoV-2 S2H13-v5 mAb VH
EVQLVESGGDSVQPGGSLRLSCAAAGFTFSSYFMNWVRQAPGKGLEWVANIKQDASEKYYVDSVKGRFTISRDNAKNSLYLQMNSLRAEDTAVYYCALSSGYSGYAGNYWGQGTLVTVSS

SEQ ID NO: 51 - protein, 17 aa, Artificial Sequence
  Synthetic sequence SARS-CoV-2 S2H13-v5 mAb CDRH2
NIKQDASEKYYVDSVKG

SEQ ID NO: 52 - protein, 120 aa, Artificial Sequence
  Synthetic sequence SARS-CoV-2 S2H13-v6 mAb VH
EVQLVESGGDSVQPGGSLRLSCAAAGFTFSSYYMNWVRQAPGKGLEWVANIKQEGSEKYYVDSVKGRFTISRDNAKNSLYLQMNSLRAEDTAVYYCALSSGYSGYAGNYWGQGTLVTVSS

SEQ ID NO: 53 - protein, 10 aa, Artificial Sequence
  Synthetic sequence SARS-CoV-2 S2H13-v6 mAb CDRH1
GFTFSSYYMN

SEQ ID NO: 54 - protein, 126 aa, Artificial Sequence
  Synthetic sequence SARS-CoV-2 S2H14-v1 mAb VH
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTGSETYYYDSSGPFDYWGQGTLVTVSS

SEQ ID NO: 55 - protein, 10 aa, Artificial Sequence
  Synthetic sequence SARS-CoV-2 S2H14-v1 mAb CDRH1
GFTFSNAWMS

SEQ ID NO: 56 - protein, 19 aa, Artificial Sequence
  Synthetic sequence SARS-CoV-2 S2H14-v1 mAb CDRH2
RIKSKTDGGTTDYAAPVKG

SEQ ID NO: 57 - protein, 15 aa, Artificial Sequence
  Synthetic sequence SARS-CoV-2 S2H14-v1 mAb CDRH3
GSETYYYDSSGPFDY

SEQ ID NO: 58 - protein, 110 aa, Artificial Sequence
  Synthetic sequence SARS-CoV-2 S2H14-v1 mAb VL
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNQVFGGGTKLTVL

SEQ ID NO: 59 - protein, 13 aa, Artificial Sequence
  Synthetic sequence SARS-CoV-2 S2H14-v1 mAb CDRL1
TRSSGSIASNYVQ

SEQ ID NO: 60 - protein, 7 aa, Artificial Sequence
  Synthetic sequence SARS-CoV-2 S2H14-v1 mAb CDRL2
EDNQRPS

SEQ ID NO: 61 - protein, 9 aa, Artificial Sequence
  Synthetic sequence SARS-CoV-2 S2H14-v1 mAb CDRL3
QSYDSSNQV

SEQ ID NO: 62 - protein, 126 aa, Artificial Sequence
  Synthetic sequence SARS-CoV-2 S2H14-v2 mAb VH
EVQLVESGGGLVKPGGSLRLSCAASGFTFSNAFMSWVRQAPGKGLEWVGRIKSKTDGGTTDYAAPVKGRFTISRDDSKNTLYLQMNSLKTEDTAVYYCTTGSETYYYDSSGPFDYWGQGTLVTVSS

SEQ ID NO: 63 - protein, 10 aa, Artificial Sequence
  Synthetic sequence SARS-CoV-2 S2H14-v2 mAb CDRH1
GFTFSNAFMS

SEQ ID NO: 64 - protein, 126 aa, Artificial Sequence
  Synthetic sequence SARS-CoV-2 S2H14-v3 mAb VH
EVQLVESGGGLVKPGGSLRLSCAASGFTFSNAWMSWVRQAPGKGLEWVGRIKSKTEGGTTDYAAPVKGRFTISRDDSKNTLYLQMNSLKTEDTAVYYCTTGSETYYYDSSGPFDYWGQGTLVTVSS

SEQ ID NO: 65 - protein, 19 aa, Artificial Sequence
  Synthetic sequence SARS-CoV-2 S2H14-v3 mAb CDRH2
RIKSKTEGGTTDYAAPVKG

SEQ ID NO: 66 - protein, 126 aa, Artificial Sequence
  Synthetic sequence SARS-CoV-2 S2H14-v4 mAb VH
EVQLVESGGGLVKPGGSLRLSCAASGFTFSNAFMSWVRQAPGKGLEWVGRIKSKTDAGTTDYAAPVKGRFTISRDDSKNTLYLQMNSLKTEDTAVYYCTTGSETYYYDSSGPFDYWGQGTLVTVSS

SEQ ID NO: 67 - protein, 19 aa, Artificial Sequence
  Synthetic sequence SARS-CoV-2 S2H14-v4 mAb CDRH2
RIKSKTDAGTTDYAAPVKG

SEQ ID NO: 68 - protein, 126 aa, Artificial Sequence
  Synthetic sequence SARS-CoV-2 S2H14-v5 mAb VH
EVQLVESGGGLVKPGGSLRLSCAASGFTFSNAFMSWVRQAPGKGLEWVGRIKSKTEGGTTDYAAPVKGRFTISRDDSKNTLYLQMNSLKTEDTAVYYCTTGSETYYYDSSGPFDYWGQGTLVTVSS

SEQ ID NO: 69 - protein, 126 aa, Artificial Sequence
  Synthetic sequence SARS-CoV-2 S2H14-v6 mAb VH
EVQLVESGGGLVKPGGSLRLSCAASGFTFSNAYMSWVRQAPGKGLEWVGRIKSKTEGGTTDYAAPVKGRFTISRDDSKNTLYLQMNSLKTEDTAVYYCTTGSETYYYDSSGPFDYWGQGTLVTVSS

SEQ ID NO: 70 - protein, 10 aa, Artificial Sequence
  Synthetic sequence SARS-CoV-2 S2H14-v6 mAb CDRH1
GFTFSNAYMS

SEQ ID NO: 71 - protein, 119 aa, Artificial Sequence
  Synthetic sequence SARS-CoV-2 S2A4-v1 mAb VH
EVQLVESGGGLVQPGGSLRLSCAASGFTFSSYWMNWVRQAPGKGLEWVANIKQDGSEKYYVDSVKGRFTISRDNAKNSLFLQMNSLRAEDTAVYYCARVWWLRGSFDYWGQGTLVTVSS

SEQ ID NO: 72 - protein, 8 aa, Artificial Sequence
  Synthetic sequence SARS-CoV-2 S2A4-v1 mAb CDRH1
GFTFSSYW

SEQ ID NO: 73 - protein, 8 aa, Artificial Sequence
  Synthetic sequence SARS-CoV-2 S2A4-v1 mAb CDRH2
IKQDGSEK

SEQ ID NO: 74 - protein, 12 aa, Artificial Sequence
  Synthetic sequence SARS-CoV-2 S2A4-v1 mAb CDRH3
ARVWWLRGSFDY

SEQ ID NO: 75 - protein, 111 aa, Artificial Sequence
  Synthetic sequence SARS-CoV-2 S2A4-v1 mAb VL
NFMLTQPHSVSESPGKTVTISCTGSSGSIASNYVQWYQQRPGSAPTTVIYEDNQRPSGVPDRFSGSIDSSSNSASLTISGLKTEDEADYYCQSYDSSNHVVFGGGTKLTVL

SEQ ID NO: 76 - protein, 8 aa, Artificial Sequence
  Synthetic sequence SARS-CoV-2 S2A4-v1 mAb CDRL1
SGSIASNY

SEQ ID NO: 77 - protein, 3 aa, Artificial Sequence
  Synthetic sequence SARS-CoV-2 S2A4-v1 mAb CDRL2
EDN

SEQ ID NO: 78 - protein, 10 aa, Artificial Sequence
  Synthetic sequence SARS-CoV-2 S2A4-v1 mAb CDRL3
QSYDSSNHVV

SEQ ID NO: 79 - DNA, 358 bp, Artificial Sequence
  Synthetic sequence SARS-CoV-2 S2A4-v1 mAb VH
gaggtgcagctggtggagtctgggggaggcttggtccagcctggggggtccctgagactctcctgtgcagcctctggattcacctttagtagctattggatgaactgggtccgccaggctccagggaaggggctggagtgggtggccaacataaagcaagatggaagtgagaaatactatgtggactctgtgaagggccgattcaccatctccagagacaacgccaagaactcactgtttctgcaaatgaacagcctgagagccgaggacacggctgtgtattactgtgcgagagtctggtggctacgaggttcctttgactactggggccagggaaccctggtcaccgtctcctcag

SEQ ID NO: 80 - DNA, 334 bp, Artificial Sequence
  Synthetic sequence SARS-CoV-2 S2A4-v1 mAb VL
aattttatgctgactcagccccactctgtgtcggagtctccggggaagacggtaaccatctcctgcaccggcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaatcatgtggtattcggcggagggaccaagctgaccgtcctag

SEQ ID NO: 81 - protein, 128 aa, Artificial Sequence
  Synthetic sequence SARS-CoV-2 S2A5-v1 mAb VH
EVQLVESGGGLVQPGRSLRLSCAASGFTFDDYAMHWVRQAPGKGLRWVSGISWNSGSIGYADSVKGRFTISRDNAKNSLYLQMNSLRAEDTALYYCAKEVGKNYYDSSGYERDYFDYWGQGTLVTVSS

SEQ ID NO: 82 - protein, 8 aa, Artificial Sequence
  Synthetic sequence SARS-CoV-2 S2A5-v1 mAb CDRH1
GFTFDDYA

SEQ ID NO: 83 - protein, 8 aa, Artificial Sequence
  Synthetic sequence SARS-CoV-2 S2A5-v1 mAb CDRH2
ISWNSGSI

SEQ ID NO: 84 - protein, 21 aa, Artificial Sequence
  Synthetic sequence SARS-CoV-2 S2A5-v1 mAb CDRH3
AKEVGKNYYDSSGYERDYFDY

SEQ ID NO: 85 - protein, 108 aa, Artificial Sequence
  Synthetic sequence SARS-CoV-2 S2A5-v1 mAb VL
SYVLTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVL

SEQ ID NO: 86 - protein, 6 aa, Artificial Sequence
  Synthetic sequence SARS-CoV-2 S2A5-v1 mAb CDRL1
NIGSKS

SEQ ID NO: 87 - protein, 3 aa, Artificial Sequence
  Synthetic sequence SARS-CoV-2 S2A5-v1 mAb CDRL2
YDS

SEQ ID NO: 88 - protein, 11 aa, Artificial Sequence
  Synthetic sequence SARS-CoV-2 S2A5-v1 mAb CDRL3
QVWDSSSDHYV

SEQ ID NO: 89 - DNA, 385 bp, Artificial Sequence
  Synthetic sequence SARS-CoV-2 S2A5-v1 mAb VH
gaagtgcagctggtggagtctgggggaggcttggtacagcctggcaggtccctgagactctcctgtgcagcctctggattcacctttgatgattatgccatgcactgggtccggcaagctccagggaagggcctgcggtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagaggtgggaaagaattactatgatagtagtggttatgaaagggactactttgactactggggccagggaaccctggtcaccgtctcctcag

SEQ ID NO: 90 - DNA, 325 bp, Artificial Sequence
  Synthetic sequence SARS-CoV-2 S2A5-v1 mAb VL
tcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggatagtagtagtgatcattatgtcttcggaactgggaccaaggtcaccgtcctag

SEQ ID NO: 91 - protein, 127 aa, Artificial Sequence
  Synthetic sequence SARS-CoV-2 S2A10-v1 mAb VH
QVQLQESGPGLVKPSQTLSLTCTVSGGSISSGDYYWSWIRQPPGKGLEWIGYIYYSGSTYYNPSLKSRVTISVDSSKNQFSLRLSSVTAADTAVYYCARGIKDAYCGGDCYHAFDIWGQGTMVTVSS

SEQ ID NO: 92 - protein, 10 aa, Artificial Sequence
  Synthetic sequence SARS-CoV-2 S2A10-v1 mAb CDRH1
GGSISSGDYY

SEQ ID NO: 93 - protein, 7 aa, Artificial Sequence
  Synthetic sequence SARS-CoV-2 S2A10-v1 mAb CDRH2
IYYSGST

SEQ ID NO: 94 - protein, 19 aa, Artificial Sequence
  Synthetic sequence SARS-CoV-2 S2A10-v1 mAb CDRH3
ARGIKDAYCGGDCYHAFDI

SEQ ID NO: 95 - protein, 109 aa, Artificial Sequence
  Synthetic sequence SARS-CoV-2 S2A10-v1 mAb VL(VK)
SYVLTQPPSVSVAPGQTARITCGGNNIGSKSVHWYQQKPGQAPVLVVYDDSDRPSGIPERFSGSNSGNTATLTISRVEAGDEADYYCQVWDSTSDHPNVFGTGTKVTVL

SEQ ID NO: 96 - protein, 6 aa, Artificial Sequence
  Synthetic sequence SARS-CoV-2 S2A10-v1 mAb CDRL1
NIGSKS

SEQ ID NO: 97 - protein, 3 aa, Artificial Sequence
  Synthetic sequence SARS-CoV-2 S2A10-v1 mAb CDRL2
DDS

SEQ ID NO: 98 - protein, 12 aa, Artificial Sequence
  Synthetic sequence SARS-CoV-2 S2A10-v1 mAb CDRL3
QVWDSTSDHPNV

SEQ ID NO: 99 - DNA, 382 bp, Artificial Sequence
  Synthetic sequence SARS-CoV-2 S2A10-v1 mAb VH
caggtgcagctgcaggagtcgggcccaggactggtgaagccttcacagaccctgtccctcacctgcactgtctctggtggctccatcagcagtggtgattactactggagttggatccgccagcccccagggaagggcctggagtggattgggtacatctattacagtgggagcacctactacaacccgtccctcaagagtcgagttaccatatcagtagactcgtccaagaaccagttctccctgaggctgagctctgtgactgccgcagacacggccgtgtattactgtgccagagggattaaggacgcatattgtggtggtgattgctaccatgcttttgatatctggggccaagggacaatggtcaccgtctcttcag

SEQ ID NO: 100 - DNA, 328 bp, Artificial Sequence
  Synthetic sequence SARS-CoV-2 S2A10-v1 mAb VL(VK)
tcctatgtgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtactagtgatcatccaaatgtcttcggaactgggaccaaggtcaccgtcctag

SEQ ID NO: 101 - protein, 123 aa, Artificial Sequence
  Synthetic sequence SARS-CoV-2 S2A15-v1 mAb VH
QVQLQESGPGLVKPSGTLSLTCAVSGGSISSSNWWSWVRQPPGKGLEWIGEIYHSGNTNYNPSLKSRVTISVDKSKNQFSLKLTSVTAADTAVYYCASRYCSSTSCPNWFDPWGQGTLVTVSS

SEQ ID NO: 102 - protein, 9 aa, Artificial Sequence
  Synthetic sequence SARS-CoV-2 S2A15-v1 mAb CDRH1
GGSISSSNW

SEQ ID NO: 103 - protein, 7 aa, Artificial Sequence
  Synthetic sequence SARS-CoV-2 S2A15-v1 mAb CDRH2
IYHSGNT

SEQ ID NO: 104 - protein, 16 aa, Artificial Sequence
  Synthetic sequence SARS-CoV-2 S2A15-v1 mAb CDRH3
ASRYCSSTSCPNWFDP

SEQ ID NO: 105 - protein, 110 aa, Artificial Sequence
  Synthetic sequence SARS-CoV-2 S2A15-v1 mAb VL
QSALTQPASVSGSPGQSITISCTGTSSDVGSYNLVSWYQQHPGKAPKFMIYEGSKRPSGVSNRFSGSKSGNTASLTISGLQAEDEADYYCCSYAGSSTWVFGGGTKLTVL

SEQ ID NO: 106 - protein, 9 aa, Artificial Sequence
  Synthetic sequence SARS-CoV-2 S2A15-v1 mAb CDRL1
SSDVGSYNL

SEQ ID NO: 107 - protein, 3 aa, Artificial Sequence
  Synthetic sequence SARS-CoV-2 S2A15-v1 mAb CDRL2
EGS

SEQ ID NO: 108 - protein, 10 aa, Artificial Sequence
  Synthetic sequence SARS-CoV-2 S2A15-v1 mAb CDRL3
CSYAGSSTWV

SEQ ID NO: 109 - DNA, 370 bp, Artificial Sequence
  Synthetic sequence SARS-CoV-2 S2A15-v1 mAb VH
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggaacaccaactacaacccgtccctcaagagtcgagtcaccatatcagtagacaagtccaagaaccagttctccctgaagctgacctctgtgaccgccgcggacacggccgtgtattactgtgcgagccgatattgtagtagtaccagttgccccaactggttcgacccctggggccagggaaccctggtcaccgtctcctcag

SEQ ID NO: 110 - DNA, 331 bp, Artificial Sequence
  Synthetic sequence SARS-CoV-2 S2A15-v1 mAb VL
cagtctgccctgactcagcctgcctccgtgtctgggtctcctggacagtcgatcaccatctcctgcactggaaccagcagtgatgttgggagttataaccttgtctcctggtaccaacagcacccaggcaaagcccccaaattcatgatttatgagggcagtaagcggccctcaggggtttctaatcgcttctctggctccaagtctggcaacacggcctccctgacaatctctgggctccaggctgaggacgaggctgattattactgctgctcatatgcaggtagtagcacttgggtgttcggcggagggaccaagctgaccgtcctag

SEQ ID NO: 111 - protein, 123 aa, Artificial Sequence
  Synthetic sequence SARS-CoV-2 S2B2-v1 mAb VH
QVQLVESGGGVVQPGRSLRLSCAASGFTFSNFGMHWVRQAPGKGLEWVAVISYDGNNKFYADSVKGRFTISRDNSKNTLYLQMNSLRAEDTAVYYCAKPTVSFGVVIDAFDIWGQGTMVTVSS

SEQ ID NO: 112 - protein, 8 aa, Artificial Sequence
  Synthetic sequence SARS-CoV-2 S2B2-v1 mAb CDRH1
GFTFSNFG

SEQ ID NO: 113 - protein, 8 aa, Artificial Sequence
  Synthetic sequence SARS-CoV-2 S2B2-v1 mAb CDRH2
ISYDGNNK

SEQ ID NO: 114 - protein, 16 aa, Artificial Sequence
  Synthetic sequence SARS-CoV-2 S2B2-v1 mAb CDRH3
AKPTVSFGVVIDAFDI

SEQ ID NO: 115 - protein, 112 aa, Artificial Sequence
  Synthetic sequence SARS-CoV-2 S2B2-v1 mAb VL(VK)
DIVMTQSPDSLAVSLGERASINCKSSQSVLYSSNNKNYLAWYQQKPGQPPKLLIYWASTRESGVPDRFSGSGSGTDFTLTISSLQAEDVAVYYCQQYYRIITFGQGTRLEIK

SEQ ID NO: 116 - protein, 12 aa, Artificial Sequence
  Synthetic sequence SARS-CoV-2 S2B2-v1 mAb CDRL1
QSVLYSSNNKNY

SEQ ID NO: 117 - protein, 3 aa, Artificial Sequence
  Synthetic sequence SARS-CoV-2 S2B2-v1 mAb CDRL2
WAS

SEQ ID NO: 118 - protein, 8 aa, Artificial Sequence
  Synthetic sequence SARS-CoV-2 S2B2-v1 mAb CDRL3
QQYYRIIT

SEQ ID NO: 119 - DNA, 370 bp, Artificial Sequence
  Synthetic sequence SARS-CoV-2 S2B2-v1 mAb VH
caggtgcagctggtggagtctgggggaggcgtggtccagcctgggaggtccctgagactctcctgtgcagcctctggattcacgttcagtaactttggcatgcactgggtccgccaggctccaggcaaggggctggagtgggtggcagttatatcatatgatggaaataataaattctatgcagactccgtgaagggccgattcaccatctccagagacaattccaagaacacgctgtatctgcaaatgaacagcctgagagctgaggacacggctgtgtattactgtgcgaaacccaccgtatcttttggagtggttattgatgcttttgatatctggggccaagggacaatggtcaccgtctcttcag

SEQ ID NO: 120 - DNA, 337 bp, Artificial Sequence
  Synthetic sequence SARS-CoV-2 S2B2-v1 mAb VL(VK)
gacatcgtgatgacccagtctccagactccctggctgtgtctctgggcgagagggccagcatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagaattatcaccttcggccaagggacacgactggagattaaac

SEQ ID NO: 121 - protein, 121 aa, Artificial Sequence
  Synthetic sequence SARS-CoV-2 S2F1-v1 mAb VH
EVQLLESGGGLVQPGGSLRLSCAGSGFSVSNHAMSWVRQAPGKGLEWVSAIGGSDSTTYYADSVKGRFAISRDNSKNTLYLQMNSLRADDTAIYYCAKDNYTSTWFPFDYWGQGTQVIVSS

SEQ ID NO: 122 - protein, 8 aa, Artificial Sequence
  Synthetic sequence SARS-CoV-2 S2F1-v1 mAb CDRH1
GFSVSNHA

SEQ ID NO: 123 - protein, 8 aa, Artificial Sequence
  Synthetic sequence SARS-CoV-2 S2F1-v1 mAb CDRH2
IGGSDSTT

SEQ ID NO: 124 - protein, 14 aa, Artificial Sequence
  Synthetic sequence SARS-CoV-2 S2F1-v1 mAb CDRH3
AKDNYTSTWFPFDY

SEQ ID NO: 125 - protein, 113 aa, Artificial Sequence
  Synthetic sequence SARS-CoV-2 S2F1-v1 mAb VL
DVVMTQSPLSLPVTLGQPASISCRSSHSLVHGDGNTYLNWFQQRPGQSPRRLIHKVSNRDSGVPDRFSGSGSGTDFTLKISRVEAEDVGVYYCMQGSYWPPWTFGQGTKVEIN

SEQ ID NO: 126 - protein, 11 aa, Artificial Sequence
  Synthetic sequence SARS-CoV-2 S2F1-v1 mAb CDRL1
HSLVHGDGNTY

SEQ ID NO: 127 - protein, 3 aa, Artificial Sequence
  Synthetic sequence SARS-CoV-2 S2F1-v1 mAb CDRL2
KVS

SEQ ID NO: 128 - protein, 10 aa, Artificial Sequence
  Synthetic sequence SARS-CoV-2 S2F1-v1 mAb CDRL3
MQGSYWPPWT

SEQ ID NO: 129 - DNA, 364 bp, Artificial Sequence
  Synthetic sequence SARS-CoV-2 S2F1-v1 mAb VH
gaggtgcaactgttggagtctgggggaggcttggtacagcctggggggtccctgagactctcctgcgcaggctctggattcagtgttagcaaccatgccatgagctgggtccgccaggctccagggaaggggctggagtgggtctcagcaattggtggaagtgacagtactacatactacgcagactctgtgaagggccggttcgccatctccagagacaattccaagaacacgctgtatctgcaaatgaacagcctgagagccgacgacacggccatatattactgtgccaaagacaactataccagtacctggttcccctttgactactggggccagggaacccaggtcatcgtctcctcag

SEQ ID NO: 130 - DNA, 340 bp, Artificial Sequence
  Synthetic sequence SARS-CoV-2 S2F1-v1 mAb VL(VK)
gatgttgtgatgactcagtctccactctctctgcccgtcacccttgggcagccggcctccatctcctgcaggtctagtcacagtctcgtacacggtgatggaaacacctacttgaattggtttcagcagaggcctggccaatctccaaggcgcctgattcataaggtttctaaccgggactctggggtccctgacagattcagcggcagtgggtcaggcactgatttcacactgaagatcagcagggtggaggctgaggatgttggggtttattactgcatgcaaggttcatattggcccccgtggacgttcggccaagggaccaaggtggaaatcaatc

SEQ ID NO: 131 - DNA, 361 bp, Artificial Sequence
  Synthetic sequence SARS-CoV-2 S2H7-v1 mAb VH
gaggtgcagctggtggagtctgggggaggcttggtccagcctggggggtccctgagactctcctgtgcagcctctggattcatcgtcagtagtaattacatgagctgggtccgccaggctccagggaaggggctggagtgggtctcagttatttatagtggtggtagcacatactacgcagactccgtgaagggcagattcaccatctccagagacaattccaagaacacgctgtatcttcaaatgaacagcctgagagccgaggacacgtctgtgtattactgtgcgagagatcggcagtatagtgggagccccagctttgactactggggccagggaaccctggtcaccgtctcctcag

SEQ ID NO: 132 - DNA, 325 bp, Artificial Sequence
  Synthetic sequence SARS-CoV-2 S2H7-v1 mAb VL(VK)
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatactgcatccagtttgcaaagtggggtcccatcaaggttcagtggcagtggatctgggacagatttcactctcaccatcagcagtctgcaacctgaagattttgcaacttactactgtcaacagagttacagtacccctgggctcactttcggcggagggaccaaggtggagatcaaac

SEQ ID NO: 133 - DNA, 361 bp, Artificial Sequence
  Synthetic sequence SARS-CoV-2 S2H13-v1 mAb VH
gaggtgcagctggtggagtctgggggagactcggtccagcctggggggtccctgagactctcctgtgcagccgctggattcacctttagtagctattggatgaactgggtccgccaggcaccagggaaggggctggagtgggtggccaacataaagcaagatggaagtgagaaatactatgtggactctgtgaagggccgattcaccatctccagagacaacgccaagaactcactgtatctacaaatgaacagcctgagagccgaggacacggccgtgtattactgtgcgctatcatcaggatatagtggctacgcaggtaactactggggccagggaaccctggtcaccgtctcctcag

SEQ ID NO: 134 - DNA, 331 bp, Artificial Sequence
  Synthetic sequence SARS-CoV-2 S2H13-v1 mAb VL
caggctgtggtgactcaggagccctcactgactgtgtccccaggagggacagtcactctcacctgtggctccagcactggagctgtcaccagtggtcattatccctactggttccagcagaagcctggccaagcccccaggacactgatttatgatacaagcaacaaacactcctggacccctgcccgattctcaggctccctccttgggggcaaagctgccctgaccctttcgggtgcgcggcctgaggatgaggctgagtattactgcttgctctcctatagtggtgctcggggggtgttcggcggagggaccaagctgaccgtcctag

SEQ ID NO: 135 - protein, 121 aa, Artificial Sequence
  Synthetic sequence SARS-CoV-2 S2R5-v1 mAb VH
QVQLVESGGGVVQPGGSLRLSCVASGFTFSDYGVNWVRQAPGKGLDWVAYIQYDGSNKYYADSVKGRFTISRDNFKNTLHLQMNSLRAEDTAVYFCAKLSVAGSFGPFDIWGQGTLVTVSS

SEQ ID NO: 136 - protein, 8 aa, Artificial Sequence
  Synthetic sequence SARS-CoV-2 S2R5-v1 mAb CDRH1
GFTFSDYG

SEQ ID NO: 137 - protein, 8 aa, Artificial Sequence
  Synthetic sequence SARS-CoV-2 S2R5-v1 mAb CDRH2
IQYDGSNK

SEQ ID NO: 138 - protein, 14 aa, Artificial Sequence
  Synthetic sequence SARS-CoV-2 S2R5-v1 mAb CDRH3
AKLSVAGSFGPFDI

SEQ ID NO: 139 - protein, 111 aa, Artificial Sequence
  Synthetic sequence SARS-CoV-2 S2R5-v1 mAb VL
QSALTQPASVSGSPGQSITISCTGTSNDVGGFNYVSWYQQHPGKAPKLLIYEVSSRPSGVSTRFSGSKSANTASLTVSGLQAEDEADYYCGSYSSTNTLVVFGSGTKVTVL

SEQ ID NO: 140 - protein, 9 aa, Artificial Sequence
  Synthetic sequence SARS-CoV-2 S2R5-v1 mAb CDRL1
SNDVGGFNY

SEQ ID NO: 141 - protein, 3 aa, Artificial Sequence
  Synthetic sequence SARS-CoV-2 S2R5-v1 mAb CDRL2
EVS

SEQ ID NO: 142 - protein, 11 aa, Artificial Sequence
  Synthetic sequence SARS-CoV-2 S2R5-v1 mAb CDRL3
GSYSSTNTLVV

SEQ ID NO: 143 - DNA, 364 bp, Artificial Sequence
  Synthetic sequence SARS-CoV-2 S2R5-v1 mAb VH
caggtgcagttggtggagtctgggggaggcgtggtccagcctggggggtccctgagactctcctgtgtagcgtccgggttcaccttcagtgactatggagttaactgggtccgccaggctccaggcaaggggctggactgggtggcatatatacaatatgatggaagtaataaatactatgcggactccgtgaagggccgattcaccatctcccgagacaatttcaagaacacgctgcatcttcaaatgaacagcctgagagccgaggacacggctgtgtatttctgtgcgaagctttcagtggctggttctttcggtccttttgatatctggggccaagggacattggtcaccgtctcttcag

SEQ ID NO: 144 - DNA, 334 bp, Artificial Sequence
  Synthetic sequence SARS-CoV-2 S2R5-v1 mAb VL
cagtctgccctgactcagcctgcctccgtgtctgggtctcctggacagtcgatcaccatctcctgcactggaaccagcaatgacgttggtggttttaactatgtctcctggtaccaacaacacccaggcaaagcccccaaactcctgatttatgaggtcagtagtcggccctcaggggtctctactcgcttctctggctccaagtctgccaacacggcctccctgaccgtctctgggctccaggctgaggacgaggctgattattactgcggctcatattcaagcaccaacactctcgttgtcttcggaagtgggaccaaggtcaccgtcctag

SEQ ID NO: 145 - protein, 122 aa, Artificial Sequence
  Synthetic sequence SARS-CoV-2 S2R7-v1 mAb VH
QVQLQESGPGLVRPSETLSLTCTVSSASIPTGTHYWGWIRQPPGQGLEWIGSISNIGYSFYNPSLKSRVSISIDTSKKQFSLKVRSVTAADTAVYYCVRPTNEYGGFWFDRWGQGTLVTVSS

SEQ ID NO: 146 - protein, 10 aa, Artificial Sequence
  Synthetic sequence SARS-CoV-2 S2R7-v1 mAb CDRH1
SASIPTGTHY

SEQ ID NO: 147 - protein, 7 aa, Artificial Sequence
  Synthetic sequence SARS-CoV-2 S2R7-v1 mAb CDRH2
ISNIGYS

SEQ ID NO: 148 - protein, 14 aa, Artificial Sequence
  Synthetic sequence SARS-CoV-2 S2R7-v1 mAb CDRH3
VRPTNEYGGFWFDR

SEQ ID NO: 149 - protein, 108 aa, Artificial Sequence
  Synthetic sequence SARS-CoV-2 S2R7-v1 mAb VL
SYVLTQPPSVSVAPGKTARFTCGGDNIGSKRVHWYQQKPGQAPVLVIYYDADRPSGIPERFSGSKSGSTATLTISRVEAGDEADYYCQVWESTRDHVIFGGGTKLTVL

SEQ ID NO: 150 - protein, 6 aa, Artificial Sequence
  Synthetic sequence SARS-CoV-2 S2R7-v1 mAb CDRL1
NIGSKR

SEQ ID NO: 151 - protein, 3 aa, Artificial Sequence
  Synthetic sequence SARS-CoV-2 S2R7-v1 mAb CDRL2
YDA

SEQ ID NO: 152 - protein, 11 aa, Artificial Sequence
  Synthetic sequence SARS-CoV-2 S2R7-v1 mAb CDRL3
QVWESTRDHVI

SEQ ID NO: 153 - DNA, 367 bp, Artificial Sequence
  Synthetic sequence SARS-CoV-2 S2R7-v1 mAb VH
caggtgcaactgcaggagtcgggcccaggactggtgaggccttcggagaccctgtcgctcacctgcactgtctctagtgcctccatccccactggtactcactattggggctggatccgccagcccccagggcagggactggagtggattgggagtatctctaatattgggtacagcttctacaacccgtccctcaagagtcgagtcagcatatccattgacacgtccaagaagcagttctccctgaaagtgaggtctgtgaccgccgcggacacggctgtctactactgtgtgagacctacgaacgaatacggtggtttctggttcgaccgctggggccagggaaccctggtcacggtctcctcag

SEQ ID NO: 154 - DNA, 325 bp, Artificial Sequence
  Synthetic sequence SARS-CoV-2 S2R7-v1 mAb VL
tcctatgtgctgactcagccaccctcagtgtcagtggcccctggaaagacggccagatttacctgtgggggagacaacattggaagtaaaagagtgcactggtaccagcagaagccaggccaggcccctgttctggtcatctattatgatgccgaccggccctcagggatccctgagcgattctctggctccaagtctgggagcacggccaccctgaccatcagcagggtcgaagccggggatgaggccgactattactgtcaggtgtgggaaagtactcgtgatcatgtgattttcggcggagggaccaagctgaccgtcctag

SEQ ID NO: 155 - protein, 123 aa, Artificial Sequence
  Synthetic sequence SARS-CoV-2 S2B2-v2 mAb VH
EVQLVESGGGVVQPGRSLRLSCAASGFTFSNFGMHWVRQAPGKGLEWVAVISYDGNNKFYADSVKGRFTISRDNSKNTLYLQMNSLRAEDTAVYYCSKPTVSFGVVIDAFDIWGQGTMVTVSS

SEQ ID NO: 156 - protein, 16 aa, Artificial Sequence
  Synthetic sequence SARS-CoV-2 S2B2-v2 mAb CDRH3
SKPTVSFGVVIDAFDI

SEQ ID NO: 157 - DNA, 370 bp, Artificial Sequence
  Synthetic sequence SARS-CoV-2 S2B2-v2 mAb VH
gaggtgcagctggtggagtctgggggaggcgtggtccagcctgggaggtccctgagactctcctgtgcagcctctggattcacgttcagtaactttggcatgcactgggtccgccaggctccaggcaaggggctggagtgggtggcagttatatcatatgatggaaataataaattctatgcagactccgtgaagggccgattcaccatctccagagacaattccaagaacacgctgtatctgcaaatgaacagcctgagagctgaggacacggctgtgtattactgttcgaaacccaccgtatcttttggagtggttattgatgcttttgatatctggggccaagggacaatggtcaccgtctcttcag

SEQ ID NO: 158 - protein, 123 aa, Artificial Sequence
  Synthetic sequence SARS-CoV-2 S2A15-v2 mAb VH
QITLKESGPGLVKPSGTLSLTCAVSGGSISSSNWWSWVRQPPGKGLEWIGEIYHSGNTNYNPSLKSRVTISVDKSKNQFSLKLTSVTAADTAVYYCASRYCSSTSCPNWFDPWGQGTLVTVSS

SEQ ID NO: 159 - DNA, 370 bp, Artificial Sequence
  Synthetic sequence SARS-CoV-2 S2A15-v2 mAb VH
cagatcaccttgaaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggaacaccaactacaacccgtccctcaagagtcgagtcaccatatcagtagacaagtccaagaaccagttctccctgaagctgacctctgtgaccgccgcggacacggccgtgtattactgtgcgagccgatattgtagtagtaccagttgccccaactggttcgacccctggggccagggaaccctggtcaccgtctcctcag

SEQ ID NO: 160 - protein, 10 aa, Artificial Sequence
  Synthetic sequence SARS-CoV-2 S2H7-v1.1 mAb CDRH1
GFIVSSNYMS

SEQ ID NO: 161 - protein, 16 aa, Artificial Sequence
  Synthetic sequence SARS-CoV-2 S2H7-v1.1 mAb CDRH2
VIYSGGSTYYADSVKG

SEQ ID NO: 162 - protein, 12 aa, Artificial Sequence
  Synthetic sequence SARS-CoV-2 S2H7-v1.1 mAb CDRH3
DRQYSGSPSFDY

SEQ ID NO: 163 - protein, 11 aa, Artificial Sequence
  Synthetic sequence SARS-CoV-2 S2H7-v1.1 mAb CDRL1
RASQSISSYLN

SEQ ID NO: 164 - protein, 7 aa, Artificial Sequence
  Synthetic sequence SARS-CoV-2 S2H7-v1.1 mAb CDRL2
TASSLQS

SEQ ID NO: 165 - protein, 10 aa, Artificial Sequence
  Synthetic sequence SARS-CoV-2 S2H7-v1.1 mAb CDRL3
QQSYSTPGLT

SEQ ID NO: 166 - protein, 10 aa, Artificial Sequence
  Synthetic sequence SARS-CoV-2 S2H13-v1.1 mAb CDRH1
GFTFSSYWMN

SEQ ID NO: 167 - protein, 17 aa, Artificial Sequence
  Synthetic sequence SARS-CoV-2 S2H13-v1.1 mAb CDRH2
NIKQDGSEKYYVDSVKG

SEQ ID NO: 168 - protein, 11 aa, Artificial Sequence
  Synthetic sequence SARS-CoV-2 S2H13-v1.1 mAb CDRH3
SSGYSGYAGNY

SEQ ID NO: 169 - protein, 14 aa, Artificial Sequence
  Synthetic sequence SARS-CoV-2 S2H13-v1.1 mAb CDRL1
GSSTGAVTSGHYPY

SEQ ID NO: 170 - protein, 7 aa, Artificial Sequence
  Synthetic sequence SARS-CoV-2 S2H13-v1.1 mAb CDRL2
DTSNKHS

SEQ ID NO: 171 - protein, 7 aa, Artificial Sequence
  Synthetic sequence SARS-CoV-2 S2H13-v1.1 mAb CDRL3
DTSNKHS

SEQ ID NO: 172 - protein, 127 aa, Artificial Sequence [sequence omitted]

SEQ ID NO: 173 - protein, 8 aa, Artificial Sequence
  Synthetic sequence SARS-CoV-2 S309 mAb CDRH1
GYPFTSYG

SEQ ID NO: 174 - protein, 8 aa, Artificial Sequence
  Synthetic sequence SARS-CoV-2 S309 mAb CDRH2
ISTYNGNT

SEQ ID NO: 175 - protein, 20 aa, Artificial Sequence
  Synthetic sequence SARS-CoV-2 S309 mAb CDRH3
ARDYTRGAWFGESLIGGFDN

SEQ ID NO: 176 - protein, 107 aa, Artificial Sequence [sequence omitted]

SEQ ID NO: 177 - protein, 7 aa, Artificial Sequence
  Synthetic sequence SARS-CoV-2 S309 mAb CDRL1
QTVSSTS

SEQ ID NO: 178 - protein, 3 aa, Artificial Sequence
  Synthetic sequence SARS-CoV-2 S309 mAb CDRL2
GAS

SEQ ID NO: 179 - protein, 8 aa, Artificial Sequence
  Synthetic sequence SARS-CoV-2 S309 mAb CDRL3
QQHDTSLT

SEQ ID NO: 180 - protein, 127 aa, Artificial Sequence
  Synthetic sequence SARS-CoV-2 S2N3-v1 mAb VH
EVQLVQSGAEVKKPGSSVKVSCKASGGVFSSYAISWVRQAPGQGLEWMGGIIPLFVTPTYAQKFQGRVTITADESTTTASMELSSLTSDDTAVYYCARDRSGYSGSWPVPNFAFHIWGQGTLVTVSS

SEQ ID NO: 181 - protein, 8 aa, Artificial Sequence
  Synthetic sequence SARS-CoV-2 S2N3-v1 mAb CDRH1
GGVFSSYA

SEQ ID NO: 182 - protein, 8 aa, Artificial Sequence
  Synthetic sequence SARS-CoV-2 S2N3-v1 mAb CDRH2
IIPLFVTP

SEQ ID NO: 183 - protein, 20 aa, Artificial Sequence
  Synthetic sequence SARS-CoV-2 S2N3-v1 mAb CDRH3
ARDRSGYSGSWPVPNFAFHI

SEQ ID NO: 184 - protein, 108 aa, Artificial Sequence
  Synthetic sequence SARS-CoV-2 S2N3-v1 mAb VL(VK)
EIVLTQSPGTLSLSPGERATLSCRASQSVSSRSLAWYQQKPGQAPRLLIYGASSRATGIPDRFSGSGSGTDFTLTISRLEPEDFAVYYCQQYGTSPRSFGPGTKVDIK

SEQ ID NO: 185 - protein, 7 aa, Artificial Sequence
  Synthetic sequence SARS-CoV-2 S2N3-v1 mAb CDRL1
QSVSSRS

SEQ ID NO: 186 - protein, 3 aa, Artificial Sequence
  Synthetic sequence SARS-CoV-2 S2N3-v1 mAb CDRL2
GAS

SEQ ID NO: 187 - protein, 9 aa, Artificial Sequence
  Synthetic sequence SARS-CoV-2 S2N3-v1 mAb CDRL3
QQYGTSPRS

SEQ ID NO: 188 - DNA, 382 bp, Artificial Sequence
  Synthetic sequence SARS-CoV-2 S2N3-v1 mAb VH
gaggtgcagctggtgcagtctggggctgaggtgaagaagcctgggtcctcggtgaaggtctcctgcaaggcttctggaggcgtcttcagcagctatgctatcagctgggtgcgacaggcccctggacaagggcttgagtggatgggggggatcatccctctctttgttacaccaacttatgcacagaagttccagggcagagtcacgattaccgcggacgaatccacgaccacagcctccatggagctgagcagcctgacatctgacgacacggccgtctattactgtgcgcgagatcgttccggatatagcggcagctggccagtcccgaactttgcttttcatatctggggccaagggacactggtcaccgtctcttcag

SEQ ID NO: 189 - DNA, 325 bp, Artificial Sequence
  Synthetic sequence SARS-CoV-2 S2N3-v1 mAb VL(VK)
gaaattgtgttgacgcagtctccaggcaccctgtctttgtctccaggggaaagagccaccctctcctgcagggccagtcagagtgttagcagcaggtccttagcctggtaccagcagaaacctggccaggctcccaggctcctcatctatggtgcatccagcagggccactggcatcccggacaggttcagtggcagtgggtctgggacagacttcactctcaccatcagcagactggagcctgaagattttgcagtgtattactgtcagcagtatggtacctcacctcggtctttcggccctgggaccaaagtggatatcaaac

SEQ ID NO: 190 - protein, 127 aa, Artificial Sequence
  Synthetic sequence SARS-CoV-2 S2N6-v1 mAb VH
QVQLVQSGAEVKRPGSSVKVSCKASGGVFSSFAISWVRQAPGQGLEWMGGIIPLFVKPDYAQKFQDRVTITADESTTTAYMELRSLKSDDTAVYYCARDHSGYSGSWPVPNYPFDIWGQGTMVTVSS

SEQ ID NO: 191 - protein, 8 aa, Artificial Sequence
  Synthetic sequence SARS-CoV-2 S2N6-v1 mAb CDRH1
GGVFSSFA

SEQ ID NO: 192 - protein, 8 aa, Artificial Sequence
  Synthetic sequence SARS-CoV-2 S2N6-v1 mAb CDRH2
IIPLFVKP

SEQ ID NO: 193 - protein, 20 aa, Artificial Sequence
  Synthetic sequence SARS-CoV-2 S2N6-v1 mAb CDRH3
ARDHSGYSGSWPVPNYPFDI

SEQ ID NO: 194 - protein, 108 aa, Artificial Sequence
  Synthetic sequence SARS-CoV-2 S2N6-v1 mAb VL(VK)
EIVLTQSPGTLSLSPGERAILSCRASQSVSSNSLAWYQQIPGQPPRLLIYGASSRATGIPDRFSGSGSGTDFTLSISRLEPEDFAVYYCQQYGGSTRSFGPGTKVDIK

SEQ ID NO: 195 - protein, 7 aa, Artificial Sequence
  Synthetic sequence SARS-CoV-2 S2N6-v1 mAb CDRL1
QSVSSNS

SEQ ID NO: 196 - protein, 3 aa, Artificial Sequence
  Synthetic sequence SARS-CoV-2 S2N6-v1 mAb CDRL2
GAS

SEQ ID NO: 197 - protein, 9 aa, Artificial Sequence
  Synthetic sequence SARS-CoV-2 S2N6-v1 mAb CDRL3
QQYGGSTRS

SEQ ID NO: 198 - DNA, 382 bp, Artificial Sequence
  Synthetic sequence SARS-CoV-2 S2N6-v1 mAb VH
caggtgcagctggtgcagtctggggctgaggtgaagaggcctgggtcctccgtgaaggtctcctgcaaggcctctggaggcgtcttcagcagctttgctatcagctgggtgcgacaggcccctggacaagggcttgagtggatgggagggatcatccctttgtttgttaaaccagactacgcacagaaattccaggacagagtcacgattaccgcggacgaatcaacgaccacagcctacatggagctgcgcagccttaaatctgacgacacggccgtttattactgtgcgagagatcattccggctatagtggcagctggccggtgccgaactatccttttgatatctggggccaagggacaatggtcaccgtctcttcag

SEQ ID NO: 199 - DNA, 325 bp, Artificial Sequence
  Synthetic sequence SARS-CoV-2 S2N6-v1 mAb VL(VK)
gaaattgtgttgacgcagtctccaggcaccctgtctttgtctcctggggaaagagccatcctctcctgcagggccagtcagagtgttagcagcaactccttagcctggtaccagcagattcctggccagcctcccaggctcctcatctacggtgcatccagcagggccactggcatcccagacaggttcagtggcagtgggtctgggacagacttcactctcagtatcagcagactggagcctgaagattttgcagtatattactgtcagcagtatggtggctcaactcggtctttcggccctgggaccaaagtggatatcaaac

SEQ ID NO: 200 - protein, 130 aa, Artificial Sequence
  Synthetic sequence SARS-CoV-2 S2X2-v1 mAb VH
QVQLVQSGAEVKKPGASVKVSCKASGYTFTSYGVSWVRQAPGQGLEWMGWISAYNGNTNYAQNLQGRVTMTTDTSTSTAYMELRSLRSDDTAVYYCARDAVITIFGVVIGKSGYYGMDVWGQGTTVTVSS

SEQ ID NO: 201 - protein, 8 aa, Artificial Sequence
  Synthetic sequence SARS-CoV-2 S2X2-v1 mAb CDRH1
GYTFTSYG

SEQ ID NO: 202 - protein, 8 aa, Artificial Sequence
  Synthetic sequence SARS-CoV-2 S2X2-v1 mAb CDRH2
ISAYNGNT

SEQ ID NO: 203 - protein, 23 aa, Artificial Sequence
  Synthetic sequence SARS-CoV-2 S2X2-v1 mAb CDRH3
ARDAVITIFGVVIGKSGYYGMDV

SEQ ID NO: 204 - protein, 113 aa, Artificial Sequence
  Synthetic sequence SARS-CoV-2 S2X2-v1 mAb VL(VK)
DVVMTQSPLSLPVTLGQPASISCRSSQSLVYSDGNTYLNWFQQRPGQSPRRLIYKVSNRDSGVPDRFSGSGSGTDFTLEISRVEAEDVGVYYCMQGTHWPAWTFGQGTKVEIK

SEQ ID NO: 205 - protein, 11 aa, Artificial Sequence
  Synthetic sequence SARS-CoV-2 S2X2-v1 mAb CDRL1
QSLVYSDGNTY

SEQ ID NO: 206 - protein, 3 aa, Artificial Sequence
  Synthetic sequence SARS-CoV-2 S2X2-v1 mAb CDRL2
KVS

SEQ ID NO: 207 - protein, 10 aa, Artificial Sequence
  Synthetic sequence SARS-CoV-2 S2X2-v1 mAb CDRL3
MQGTHWPAWT

SEQ ID NO: 208 - DNA, 390 bp, Artificial Sequence
  Synthetic sequence SARS-CoV-2 S2X2-v1 mAb VH
caggtgcagctggtgcagtctggagctgaggtgaagaagcctggggcctcagtgaaggtctcctgcaaggcttctggttacacctttaccagctatggtgtcagctgggtgcgacaggcccctggacaagggcttgagtggatgggatggatcagcgcttacaatggtaacacaaactatgcacagaacctccagggcagagtcaccatgaccacagacacatccacgagcacagcctacatggagctgaggagcctgagatctgacgacacggccgtgtattactgtgcgagagatgccgtgattacgatttttggagtggttattgggaaatcgggctattacggtatggacgtctggggccaagggaccacggtcaccgtctcctca

SEQ ID NO: 209 - DNA, 340 bp, Artificial Sequence
  Synthetic sequence SARS-CoV-2 S2X2-v1 mAb VL(VK)
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactggaaatcagcagggtggaggctgaggatgttggggtttattactgcatgcaaggtacacactggcctgcgtggacgttcggccaagggaccaaggtggagatcaaac

SEQ ID NO: 210 - protein, 122 aa, Artificial Sequence
  Synthetic sequence SARS-CoV-2 S2D8-v1 mAb VH
EVQLLESGGGLVQPGGSLRLSCAASGFTFTTYAMSWVRQAPGKGLEWVSGISGSGGNTYHADSVKGRFTISRDNSKSTLYLQMNSLRAEDTAVYYCAKDLWFREILHGMDVWGEGTTVTVSS

SEQ ID NO: 211 - protein, 8 aa, Artificial Sequence
  Synthetic sequence SARS-CoV-2 S2D8-v1 mAb CDRH1
GFTFTTYA

SEQ ID NO: 212 - protein, 8 aa, Artificial Sequence
  Synthetic sequence SARS-CoV-2 S2D8-v1 mAb CDRH2
ISGSGGNT

SEQ ID NO: 213 - protein, 15 aa, Artificial Sequence
  Synthetic sequence SARS-CoV-2 S2D8-v1 mAb CDRH3
AKDLWFREILHGMDV

SEQ ID NO: 214 - protein, 110 aa, Artificial Sequence
  Synthetic sequence SARS-CoV-2 S2D8-v1 mAb VL
QSALTQPASVSGSPGQSITISCTGTSSDIGGYNYVSWYQHHPGKAPKIMIYEVTNRPSGVSNRFSGSKSGNTASLTISGLQAEDEADYYCSSYTSSNTYVFGTGTKVTVL

SEQ ID NO: 215 - protein, 9 aa, Artificial Sequence
  Synthetic sequence SARS-CoV-2 S2D8-v1 mAb CDRL1
SSDIGGYNY

SEQ ID NO: 216 - protein, 3 aa, Artificial Sequence
  Synthetic sequence SARS-CoV-2 S2D8-v1 mAb CDRL2
EVT

SEQ ID NO: 217 - protein, 10 aa, Artificial Sequence
  Synthetic sequence SARS-CoV-2 S2D8-v1 mAb CDRL3
SSYTSSNTYV

SEQ ID NO: 218 - DNA, 367 bp, Artificial Sequence
  Synthetic sequence SARS-CoV-2 S2D8-v1 mAb VH
gaggtgcagctgttggagtctgggggaggcttggtgcagcctggggggtccctgagactctcttgtgcagcctctggattcacctttaccacctatgccatgagttgggtccgccaggctccagggaaggggctggagtgggtctcaggtattagtggtagtggtggtaacacataccacgcagactccgtgaagggccggttcaccatctccagagacaattccaagagcacgctgtatctgcaaatgaacagcctgagagccgaggacacggccgtgtattactgtgcgaaagacctctggttcagggagatactccacggtatggatgtctggggcgaagggaccacggtcaccgtctcctcag

SEQ ID NO: 219 - DNA, 331 bp, Artificial Sequence
  Synthetic sequence SARS-CoV-2 S2D8-v1 mAb VL
cagtctgccctgactcagcctgcctccgtgtctgggtctcctggacagtcgatcaccatctcctgcactggaaccagcagtgacattggtggttataactatgtctcctggtaccaacaccacccaggcaaagcccccaaaataatgatttatgaggtcactaatcggccctcaggggtttctaatcgcttctctggctccaagtctggcaacacggcctccctgaccatctctgggctccaggctgaggacgaggctgattattactgcagctcatatacaagcagcaacacttatgtcttcggaactgggaccaaggtcaccgtcctag

SEQ ID NO: 220 - protein, 122 aa, Artificial Sequence
  Synthetic sequence SARS-CoV-2 S2D25-v1 mAb VH
EVQLVESGGGLVQPGGSLRLSCAASGFPFNIYAMSWVRQAPGKGLEWVSGISGSGGSTYYADSVRGRFAISRDNSKNTLYLQMNSLRAEDTAEYYCAKDLWFREILHGMDVWGKGTTVTVSS

SEQ ID NO: 221 - protein, 8 aa, Artificial Sequence
  Synthetic sequence SARS-CoV-2 S2D25-v1 mAb CDRH1
GFPFNIYA

SEQ ID NO: 222 - protein, 8 aa, Artificial Sequence
  Synthetic sequence SARS-CoV-2 S2D25-v1 mAb CDRH2
ISGSGGST

SEQ ID NO: 223 - protein, 15 aa, Artificial Sequence
  Synthetic sequence SARS-CoV-2 S2D25-v1 mAb CDRH3
AKDLWFREILHGMDV

SEQ ID NO: 224 - protein, 110 aa, Artificial Sequence
  Synthetic sequence SARS-CoV-2 S2D25-v1 mAb VL
QSALTQPASVSGSPGQSITISCTGTSSDVGGYNYVSWYQHHPGRAPKLMIYEVSNRPSGVSNRFSGSKSGNTASLSISGLQAEDEADYYCSSYTSSSTYVFGAGTKVTVL

SEQ ID NO: 225 - protein, 9 aa, Artificial Sequence
  Synthetic sequence SARS-CoV-2 S2D25-v1 mAb CDRL1
SSDVGGYNY

SEQ ID NO: 226 - protein, 3 aa, Artificial Sequence
  Synthetic sequence SARS-CoV-2 S2D25-v1 mAb CDRL2
EVS

SEQ ID NO: 227 - protein, 10 aa, Artificial Sequence
  Synthetic sequence SARS-CoV-2 S2D25-v1 mAb CDRL3
SSYTSSSTYV

SEQ ID NO: 228 - DNA, 367 bp, Artificial Sequence
  Synthetic sequence SARS-CoV-2 S2D25-v1 mAb VH
gaggtgcagctggtggagtctgggggaggcttggtacagcctggggggtccctgagactctcctgtgcagcctctggattcccatttaacatctatgccatgagctgggtccgccaggctccagggaaggggctggagtgggtctcaggtattagtggaagtggtggtagcacatactacgcagactccgtgaggggccggttcgccatctccagagacaattccaagaacacgctgtatctgcaaatgaacagcctgagagccgaggacacggccgaatattactgtgcgaaagacctctggttcagggagatactccacggtatggacgtctggggcaaagggaccacggtcaccgtctcctcag

SEQ ID NO: 229 - DNA, 331 bp, Artificial Sequence
  Synthetic sequence SARS-CoV-2 S2D25-v1 mAb VL
cagtctgacctgactcagcctgcctccgtgtctgggtctcctggacagtcgatcaccatctcctgcactggaaccagcagtgacgttggtggttataactatgtctcctggtaccaacaccacccaggcagagcccccaaactcatgatttatgaggtcagtaatcggccctcaggggtttctaatcgcttctctggctccaagtctggcaacacggcctccctgagcatctctgggctccaggctgaggacgaggctgattattactgcagctcatatacaagcagcagcacttatgtcttcggagctgggaccaaggtcaccgtcctag

SEQ ID NO: 230 - protein, 110 aa, Artificial Sequence
  Synthetic sequence SARS-CoV-2 S2D25-v2 mAb VL
QSDLTQPASVSGSPGQSITISCTGTSSDVGGYNYVSWYQHHPGRAPKLMIYEVSNRPSGVSNRFSGSKSGNTASLSISGLQAEDEADYYCSSYTSSSTYVFGAGTKVTVL

SEQ ID NO: 231 - DNA, 331 bp, Artificial Sequence
  Synthetic sequence SARS-CoV-2 S2D25-v2 mAb VL
cagtctgccctgactcagcctgcctccgtgtctgggtctcctggacagtcgatcaccatctcctgcactggaaccagcagtgacgttggtggttataactatgtctcctggtaccaacaccacccaggcagagcccccaaactcatgatttatgaggtcagtaatcggccctcaggggtttctaatcgcttctctggctccaagtctggcaacacggcctccctgagcatctctgggctccaggctgaggacgaggctgattattactgcagctcatatacaagcagcagcacttatgtcttcggagctgggaccaaggtcaccgtcctag

SEQ ID NO: 232 - protein, 128 aa, Artificial Sequence
  Synthetic sequence SARS-CoV-2 S2D32-v1 mAb VH
EVQLVESGGGLVQPGRSLRLSCTSSGFTFGDYPMSWFRQAPGKGLEWVGFIRSKAYGGTTQYAASVKGRFTISRDDSKNIAYLQMNSLKTEDTAVYYCTREMWDCSGGRCYSPFFDYWGQGTLVTVSS

SEQ ID NO: 233 - protein, 8 aa, Artificial Sequence
  Synthetic sequence SARS-CoV-2 S2D32-v1 mAb CDRH1
GFTFGDYP

SEQ ID NO: 234 - protein, 10 aa, Artificial Sequence
  Synthetic sequence SARS-CoV-2 S2D32-v1 mAb CDRH2
IRSKAYGGTT

SEQ ID NO: 235 - protein, 19 aa, Artificial Sequence
  Synthetic sequence SARS-CoV-2 S2D32-v1 mAb CDRH3
TREMWDCSGGRCYSPFFDY

SEQ ID NO: 236 - protein, 106 aa, Artificial Sequence
  Synthetic sequence SARS-CoV-2 S2D32-v1 mAb VL (VK)
EIVMTQSPATLSVSPGERATLSCRASQTVSSNLAWYQQKPGQAPRLLIYGASTRATGIPARFSGSGSGTEFTLTISSLQSEDFAVYYCQQYNNWRTFGQGTKLEIK

SEQ ID NO: 237 - protein, 6 aa, Artificial Sequence
  Synthetic sequence SARS-CoV-2 S2D32-v1 mAb CDRL1
QTVSSN

SEQ ID NO: 238 - protein, 3 aa, Artificial Sequence
  Synthetic sequence SARS-CoV-2 S2D32-v1 mAb CDRL2
GAS

SEQ ID NO: 239 - protein, 8 aa, Artificial Sequence
  Synthetic sequence SARS-CoV-2 S2D32-v1 mAb CDRL3
QQYNNWRT

SEQ ID NO: 240 - DNA, 385 bp, Artificial Sequence
  Synthetic sequence SARS-CoV-2 S2D32-v1 mAb VH
gaggtgcagctggtggagtctgggggaggcttggtacagccagggcggtccctgagactctcctgtacatcttctggattcacctttggtgattatcctatgagctggttccgccaggctccagggaaggggctggagtgggtaggtttcattagaagcaaagcttatggtgggacaacacaatacgccgcgtctgtgaaaggcagattcaccatctcaagagatgactccaaaaacatcgcctatctgcaaatgaacagcctgaaaaccgaggacacagccgtgtattactgtactagagaaatgtgggattgtagtggtggtaggtgctactccccttttttcgactactggggccagggaaccctggtcaccgtctcctcag

SEQ ID NO: 241 - DNA, 319 bp, Artificial Sequence
  Synthetic sequence SARS-CoV-2 S2D32-v1 mAb VL(VK)
gaaatagtgatgacgcagtctccagccaccctgtctgtgtctccaggggaaagagccaccctctcctgcagggccagtcagactgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcggacttttggccaggggaccaagctggagatcaaac

SEQ ID NO: 242 - protein, 123 aa, Artificial Sequence
  Synthetic sequence SARS-CoV-2 S2D60-v1 mAb VH
EVQLVQSGPEVKKPGTSVKVSCKASGFTFMSSAVQWVRQARGQRLEWIGWIVVGSGNTNYTQKFRERVTITRDMSTSTAYMELSSLRSEDTAVYYCAAPRCSGGSCHDGFDIWGQGTMVTVSS

SEQ ID NO: 243 - protein, 8 aa, Artificial Sequence
  Synthetic sequence SARS-CoV-2 S2D60-v1 mAb CDRH1
GFTFMSSA

SEQ ID NO: 244 - protein, 8 aa, Artificial Sequence
  Synthetic sequence SARS-CoV-2 S2D60-v1 mAb CDRH2
IVVGSGNT

SEQ ID NO: 245 - protein, 16 aa, Artificial Sequence
  Synthetic sequence SARS-CoV-2 S2D60-v1 mAb CDRH3
AAPRCSGGSCHDGFDI

SEQ ID NO: 246 - protein, 108 aa, Artificial Sequence
  Synthetic sequence SARS-CoV-2 S2D60-v1 mAb VL (VK)
EIVLTQSPGTLSLSPGERATLSCRASQSVSSSYLGWYQQKPGQAPRLLIYGASSRATGIPDRFSGSGSGTDFTLTISRLEPEDFAVYYCQQYGRSPWTFGQGTKVEIK

SEQ ID NO: 247 - protein, 7 aa, Artificial Sequence
  Synthetic sequence SARS-CoV-2 S2D60-v1 mAb CDRL1
QSVSSSY

SEQ ID NO: 248 - protein, 3 aa, Artificial Sequence
  Synthetic sequence SARS-CoV-2 S2D60-v1 mAb CDRL2
GAS

SEQ ID NO: 249 - protein, 9 aa, Artificial Sequence
  Synthetic sequence SARS-CoV-2 S2D60-v1 mAb CDRL3
QQYGRSPWT

SEQ ID NO: 250 - DNA, 370 bp, Artificial Sequence
  Synthetic sequence SARS-CoV-2 S2D60-v1 mAb VH
gaggtgcagctggtgcagtctgggcctgaggtgaagaagcctgggacctcagtgaaggtctcctgcaaggcttctggattcacctttatgagctctgctgtgcagtgggtgcgacaggctcgtggacaacgccttgagtggataggatggatcgtcgttggcagtggtaacacaaactacacacagaagttccgggaaagagtcaccatcaccagggacatgtccacaagtacagcctacatggagctgagcagcctgagatccgaggacacggccgtgtattattgtgcggctcctcgttgtagtggtggtagctgccatgatggttttgatatctggggccaagggacaatggtcaccgtctcttcag

SEQ ID NO: 251 - DNA, 325 bp, Artificial Sequence
  Synthetic sequence SARS-CoV-2 S2D60-v1 mAb VL (VK)
gaaattgtgttgacgcagtctccaggcaccctgtctttgtctccaggggaaagagccaccctctcctgcagggccagtcagagtgttagcagcagctacttaggctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtaggtcaccgtggacgttcggccaagggaccaaggtggagatcaaac

SEQ ID NO: 252 - protein, 122 aa, Artificial Sequence
  Synthetic sequence SARS-CoV-2 S2D8-v2 mAb VH
EVQLLESGGGLVQPGGSLRLSCAASGFTFTTYAMSWVRQAPGKGLEWVSGISGSGGNTYHAESVKGRFTISRDNSKSTLYLQMNSLRAEDTAVYYCAKDLWFREILHGMDVWGEGTTVTVSS

SEQ ID NO: 253 - protein, 122 aa, Artificial Sequence
  Synthetic sequence SARS-CoV-2 S2D8-v3 mAb VH
EVQLLESGGGLVQPGGSLRLSCAASGFTFTTYAMSWVRQAPGKGLEWVSGISGSGGNTYHADSVKGRFTISRDNSKSTLYLQMNSLRAEDTAVYYCAKDLFFREILHGMDVWGEGTTVTVSS

SEQ ID NO: 254 - protein, 13 aa, Artificial Sequence
  Synthetic sequence SARS-CoV-2 S2D8-v3 mAb CDRH3
DLFFREILHGMDV

SEQ ID NO: 255 - protein, 122 aa, Artificial Sequence
  Synthetic sequence SARS-CoV-2 S2D8-v4 mAb VH
EVQLLESGGGLVQPGGSLRLSCAASGFTFTTYAMSWVRQAPGKGLEWVSGISGSGGNTYHAESVKGRFTISRDNSKSTLYLQMNSLRAEDTAVYYCAKDLFFREILHGMDVWGEGTTVTVSS

SEQ ID NO: 256 - protein, 122 aa, Artificial Sequence
  Synthetic sequence SARS-CoV-2 S2D8-v5 mAb VH
EVQLLESGGGLVQPGGSLRLSCAASGFTFTTYAMSWVRQAPGKGLEWVSGISGSGGNTYHADAVKGRFTISRDNSKSTLYLQMNSLRAEDTAVYYCAKDLWFREALHGMDVWGEGTTVTVSS

SEQ ID NO: 257 - protein, 13 aa, Artificial Sequence
  Synthetic sequence SARS-CoV-2 S2D8-v5 mAb CDRH3
DLWFREALHGMDV

SEQ ID NO: 258 - protein, 122 aa, Artificial Sequence
  Synthetic sequence SARS-CoV-2 S2D8-v6 mAb VH
EVQLLESGGGLVQPGGSLRLSCAASGFTFTTYAMSWVRQAPGKGLEWVSGISGSGGNTYHADAVKGRFTISRDNSKSTLYLQMNSLRAEDTAVYYCAKDLFFREALHGMDVWGEGTTVTVSS

SEQ ID NO: 259 - protein, 13 aa, Artificial Sequence
  Synthetic sequence SARS-CoV-2 S2D8-v6 mAb CDRH3
DLFFREALHGMDV

SEQ ID NO: 260 - protein, 122 aa, Artificial Sequence
  Synthetic sequence SARS-CoV-2 S2D8-v7 mAb VH
EVQLLESGGGLVQPGGSLRLSCAASGFTFTTYAMSWVRQAPGKGLEWVSGISGSGGNTYHADAVKGRFTISRDNSKSTLYLQMNSLRAEDTAVYYCAKDLYFREALHGMDVWGEGTTVTVSS

SEQ ID NO: 261 - protein, 13 aa, Artificial Sequence
  Synthetic sequence SARS-CoV-2 S2D8-v7 mAb CDRH3
DLYFREALHGMDV

SEQ ID NO: 262 - protein, 122 aa, Artificial Sequence
  Synthetic sequence SARS-CoV-2 S2D25-v3 mAb VH
EVQLVESGGGLVQPGGSLRLSCAASGFPFNIYAMSWVRQAPGKGLEWVSGISGSGGSTYYAESVRGRFAISRDNSKNTLYLQMNSLRAEDTAEYYCAKDLWFREILHGMDVWGKGTTVTVSS

SEQ ID NO: 263 - protein, 17 aa, Artificial Sequence
  Synthetic sequence SARS-CoV-2 S2D25-v3 mAb CDRH2
GISGSGGSTYYAESVRG

SEQ ID NO: 264 - protein, 122 aa, Artificial Sequence
  Synthetic sequence SARS-CoV-2 S2D25-v4 mAb VH
EVQLVESGGGLVQPGGSLRLSCAASGFPFNIYAMSWVRQAPGKGLEWVSGISGSGGSTYYADSVRGRFAISRDNSKNTLYLQMNSLRAEDTAEYYCAKDLFFREILHGMDVWGKGTTVTVSS

SEQ ID NO: 265 - protein, 13 aa, Artificial Sequence
  Synthetic sequence SARS-CoV-2 S2D25-v4 mAb CDRH3
DLFFREILHGMDV

SEQ ID NO: 266 - protein, 122 aa, Artificial Sequence
  Synthetic sequence SARS-CoV-2 S2D25-v5 mAb VH
EVQLVESGGGLVQPGGSLRLSCAASGFPFNIYAMSWVRQAPGKGLEWVSGISGSGGSTYYAESVRGRFAISRDNSKNTLYLQMNSLRAEDTAEYYCAKDLFFREILHGMDVWGKGTTVTVSS

SEQ ID NO: 267 - protein, 122 aa, Artificial Sequence
  Synthetic sequence SARS-CoV-2 S2D25-v6 mAb VH
EVQLVESGGGLVQPGGSLRLSCAASGFPFNSYAMSWVRQAPGKGLEWVSGISGSGGSTYYADAVRGRFAISRDNSKNTLYLQMNSLRAEDTAEYYCAKDLFFREILHGMDVWGKGTTVTVSS

SEQ ID NO: 268 - protein, 10 aa, Artificial Sequence
  Synthetic sequence SARS-CoV-2 S2D25-v6 mAb CDRH1
GFPFNSYAMS

SEQ ID NO: 269 - protein, 17 aa, Artificial Sequence
  Synthetic sequence SARS-CoV-2 S2D25-v6 mAb CDRH2
GISGSGGSTYYADAVRG

SEQ ID NO: 270 - protein, 122 aa, Artificial Sequence
  Synthetic sequence SARS-CoV-2 S2D25-v7 mAb VH
EVQLVESGGGLVQPGGSLRLSCAASGFPFNSYAMSWVRQAPGKGLEWVSGISGSGGSTYYADAVRGRFAISRDNSKNTLYLQMNSLRAEDTAEYYCAKDLWFREALHGMDVWGKGTTVTVSS

SEQ ID NO: 271 - protein, 13 aa, Artificial Sequence
  Synthetic sequence SARS-CoV-2 S2D25-v7 mAb CDRH3
DLWFREALHGMDV

SEQ ID NO: 272 - protein, 122 aa, Artificial Sequence
  Synthetic sequence SARS-CoV-2 S2D25-v8 mAb VH
EVQLVESGGGLVQPGGSLRLSCAASGFPFNSYAMSWVRQAPGKGLEWVSGISGSGGSTYYADAVRGRFAISRDNSKNTLYLQMNSLRAEDTAEYYCAKDLYFREILHGMDVWGKGTTVTVSS

SEQ ID NO: 273 - protein, 13 aa, Artificial Sequence
  Synthetic sequence SARS-CoV-2 S2D25-v8 mAb CDRH3
DLYFREILHGMDV

SEQ ID NO: 274 - protein, 128 aa, Artificial Sequence
  Synthetic sequence SARS-CoV-2 S2D32-v2 mAb VH
EVQLVESGGGLVQPGRSLRLSCTSSGFTFGDYPMSWFRQAPGKGLEWVGFIRSKAYGGTTQYAASVKGRFTISRDDSKNIAYLQMNSLKTEDTAVYYCTREMFDCSGGRCYSPFFDYWGQGTLVTVSS

SEQ ID NO: 275 - protein, 17 aa, Artificial Sequence
  Synthetic sequence SARS-CoV-2 S2D32-v2 mAb CDRH3
EMFDCSGGRCYSPFFDY

SEQ ID NO: 276 - protein, 128 aa, Artificial Sequence
  Synthetic sequence SARS-CoV-2 S2D32-v3 mAb VH
EVQLVESGGGLVQPGRSLRLSCTSSGFTFGDYPMSWFRQAPGKGLEWVGFIRSKAYGGTTQYAASVKGRFTISRDDSKNIAYLQMNSLKTEDTAVYYCTREMWDSSGGRSYSPFFDYWGQGTLVTVSS

SEQ ID NO: 277 - protein, 17 aa, Artificial Sequence
  Synthetic sequence SARS-CoV-2 S2D32-v3 mAb CDRH3
EMWDSSGGRSYSPFFDY

SEQ ID NO: 278 - protein, 128 aa, Artificial Sequence
  Synthetic sequence SARS-CoV-2 S2D32-v4 mAb VH
EVQLVESGGGLVQPGRSLRLSCTSSGFTFGDYPMSWFRQAPGKGLEWVGFIRSKAYGGTTQYAASVKGRFTISRDDSKNIAYLQMNSLKTEDTAVYYCTREMWDPSGGRPYSPFFDYWGQGTLVTVSS

SEQ ID NO: 279 - protein, 17 aa, Artificial Sequence
  Synthetic sequence SARS-CoV-2 S2D32-v4 mAb CDRH3
EMWDPSGGRPYSPFFDY

SEQ ID NO: 280 - protein, 128 aa, Artificial Sequence
  Synthetic sequence SARS-CoV-2 S2D32-v5 mAb VH
EVQLVESGGGLVQPGRSLRLSCTSSGFTFGDYPMSWFRQAPGKGLEWVGFIRSKAYGGTTQYAASVKGRFTISRDDSKNIAYLQMNSLKTEDTAVYYCTREMWDASGGRAYSPFFDYWGQGTLVTVSS

SEQ ID NO: 281 - protein, 17 aa, Artificial Sequence
  Synthetic sequence SARS-CoV-2 S2D32-v5 mAb CDRH3
EMWDASGGRAYSPFFDY

SEQ ID NO: 282 - protein, 106 aa, Artificial Sequence
  Synthetic sequence SARS-CoV-2 S2D32-v6 mAb VL(VK)
EIVMTQSPATLSVSPGERATLSCRASQTVSSNLAWYQQKPGQAPRLLIYGASTRATGIPARFSGSGSGTEFTLTISSLQSEDFAVYYCQQYNNFRTFGQGTKLEIK

SEQ ID NO: 283 - protein, 8 aa, Artificial Sequence
  Synthetic sequence SARS-CoV-2 S2D32-v6 mAb CDRL3
QQYNNFRT

SEQ ID NO: 284 - protein, 123 aa, Artificial Sequence
  Synthetic sequence SARS-CoV-2 S2D60-v2 mAb VH
EVQLVQSGPEVKKPGTSVKVSCKASGFTFMSSAVQWVRQARGQRLEWIGFIVVGSGNTNYAQKFRERVTITRDMSTSTAYMELSSLRSEDTAVYYCAAPRCSGGSCHDGFDIWGQGTMVTVSS

SEQ ID NO: 285 - protein, 17 aa, Artificial Sequence
  Synthetic sequence SARS-CoV-2 S2D60-v2 mAb CDRH2
FIVVGSGNTNYAQKFRE

SEQ ID NO: 286 - protein, 123 aa, Artificial Sequence
  Synthetic sequence SARS-CoV-2 S2D60-v3 mAb VH
EVQLVQSGPEVKKPGTSVKVSCKASGFTFMSSAVQWVRQARGQRLEWIGFIVVGSGNTQYTQKFRERVTITRDMSTSTAYMELSSLRSEDTAVYYCAAPRCSGGSCHDGFDIWGQGTMVTVSS

SEQ ID NO: 287 - protein, 17 aa, Artificial Sequence
  Synthetic sequence SARS-CoV-2 S2D60-v3 mAb CDRH2
FIVVGSGNTQYTQKFRE

SEQ ID NO: 288 - protein, 123 aa, Artificial Sequence
  Synthetic sequence SARS-CoV-2 S2D60-v4 mAb VH
EVQLVQSGPEVKKPGTSVKVSCKASGFTFMSSAVQWVRQARGQRLEWIGWIVVGSGNTNYAQKFRERVTITRDMSTSTAYMELSSLRSEDTAVYYCAAPRCSGGSCHDAFDIWGQGTMVTVSS

SEQ ID NO: 289 - protein, 17 aa, Artificial Sequence
  Synthetic sequence SARS-CoV-2 S2D60-v4 mAb CDRH2
WIVVGSGNTNYAQKFRE

SEQ ID NO: 290 - protein, 14 aa, Artificial Sequence
  Synthetic sequence SARS-CoV-2 S2D60-v4 mAb CDRH3
PRCSGGSCHDAFDI

SEQ ID NO: 291 - protein, 123 aa, Artificial Sequence
  Synthetic sequence SARS-CoV-2 S2D60-v5 mAb VH
EVQLVQSGPEVKKPGTSVKVSCKASGFTFMSSAVQWVRQARGQRLEWIGFIVVGSGNTNYAQKFRERVTITRDMSTSTAYMELSSLRSEDTAVYYCAAPRCSGGSCHDAFDIWGQGTMVTVSS

SEQ ID NO: 292 - protein, 123 aa, Artificial Sequence
  Synthetic sequence SARS-CoV-2 S2D60-v6 mAb VH
EVQLVQSGPEVKKPGTSVKVSCKASGFTFMSSAVQWVRQARGQRLEWIGYIVVGSGNTQYTQKFRERVTITRDMSTSTAYMELSSLRSEDTAVYYCAAPRCSGGSCHEGFDIWGQGTMVTVSS

SEQ ID NO: 293 - protein, 17 aa, Artificial Sequence
  Synthetic sequence SARS-CoV-2 S2D60-v6 mAb CDRH2
YIVVGSGNTQYTQKFRE

SEQ ID NO: 294 - protein, 14 aa, Artificial Sequence
  Synthetic sequence SARS-CoV-2 S2D60-v6 mAb CDRH3
PRCSGGSCHEGFDI

SEQ ID NO: 295 - protein, 123 aa, Artificial Sequence
  Synthetic sequence SARS-CoV-2 S2D60-v7 mAb VH
EVQLVQSGPEVKKPGTSVKVSCKASGFTFMSSAVQWVRQARGQRLEWIGWIVVGSGNTNYAQKFRERVTITRDMSTSTAYMELSSLRSEDTAVYYCAAPRSSGGSSHDGFDIWGQGTMVTVSS

SEQ ID NO: 296 - protein, 14 aa, Artificial Sequence
  Synthetic sequence SARS-CoV-2 S2D60-v7 mAb CDRH3
PRSSGGSSHDGFDI

SEQ ID NO: 297 - protein, 123 aa, Artificial Sequence
  Synthetic sequence SARS-CoV-2 S2D60-v8 mAb VH
EVQLVQSGPEVKKPGTSVKVSCKASGFTFMSSAVQWVRQARGQRLEWIGWIVVGSGNTNYAQKFRERVTITRDMSTSTAYMELSSLRSEDTAVYYCAAPRCSGGSCHDGFDIWGQGTMVTVSS

SEQ ID NO: 298 - protein, 123 aa, Artificial Sequence
  Synthetic sequence SARS-CoV-2 S2D60-v9 mAb VH
EVQLVQSGPEVKKPGTSVKVSCKASGFTFMSSAVQWVRQARGQRLEWIGWIVVGSGNTQYTQKFRERVTITRDMSTSTAYMELSSLRSEDTAVYYCAAPRCSGGSCHDGFDIWGQGTMVTVSS

SEQ ID NO: 299 - protein, 17 aa, Artificial Sequence
  Synthetic sequence SARS-CoV-2 S2D60-v9 mAb CDRH2
WIVVGSGNTQYTQKFRE

SEQ ID NO: 300 - protein, 123 aa, Artificial Sequence
  Synthetic sequence SARS-CoV-2 S2D60-v10 mAb VH
EVQLVQSGPEVKKPGTSVKVSCKASGFTFMSSAVQWVRQARGQRLEWIGWIVVGSGNTDYTQKFRERVTITRDMSTSTAYMELSSLRSEDTAVYYCAAPRCSGGSCHDGFDIWGQGTMVTVSS

SEQ ID NO: 301 - protein, 17 aa, Artificial Sequence
  Synthetic sequence SARS-CoV-2 S2D60-v10 mAb CDRH2
WIVVGSGNTDYTQKFRE

SEQ ID NO: 302 - protein, 108 aa, Artificial Sequence
  Synthetic sequence SARS-CoV-2 S2D60-v11 mAb VL(VK)
EIVLTQSPGTLSLSPGERATLSCRASQSVSSSYLGWYQQKPGQAPRLLIYGASSRATGIPDRFSGSGSGTDFTLTISRLEPEDFAVYYCQQYGRSPFTFGQGTKVEIK

SEQ ID NO: 303 - protein, 9 aa, Artificial Sequence
  Synthetic sequence SARS-CoV-2 S2D60-v11 mAb CDRL3
QQYGRSPFT

SEQ ID NO: 304 - protein, 129 aa, Artificial Sequence
  Synthetic sequence SARS-CoV-2 S2D22-v1 mAb VH
EVHLVESGGGLVQPGGSLKLSCAASGFTFSGSAMHWVRQASGKGLEWVGRIRTKANTYATAYAASVKGRFTISRDDSKNTAYLQMNSLKTEDTAVYYCTRPAPYDFLSDYYTGEQLDYWGQGTLVTVSS

SEQ ID NO: 305 - protein, 8 aa, Artificial Sequence
  Synthetic sequence SARS-CoV-2 S2D22-v1 mAb CDRH1
GFTFSGSA

SEQ ID NO: 306 - protein, 10 aa, Artificial Sequence
  Synthetic sequence SARS-CoV-2 S2D22-v1 mAb CDRH2
IRTKANTYAT

SEQ ID NO: 307 - protein, 20 aa, Artificial Sequence
  Synthetic sequence SARS-CoV-2 S2D22-v1 mAb CDRH3
TRPAPYDFLSDYYTGEQLDY

SEQ ID NO: 308 - protein, 112 aa, Artificial Sequence
  Synthetic sequence SARS-CoV-2 S2D22-v1 mAb VL
QSVLTQPPSVSGAPGQRVTISCTGSSSNIGAGYDVHWYQQLPGAAPKLLIYGNSNRPSGVPDRFSGSKSGTSASLAITGLQAEDEADYYCQSYDSSLSGPEVFGTGTKVTVL

SEQ ID NO: 309 - protein, 9 aa, Artificial Sequence
  Synthetic sequence SARS-CoV-2 S2D22-v1 mAb CDRL1
SSNIGAGYD

SEQ ID NO: 310 - protein, 3 aa, Artificial Sequence
  Synthetic sequence SARS-CoV-2 S2D22-v1 mAb CDRL2
GNS

SEQ ID NO: 311 - protein, 12 aa, Artificial Sequence
  Synthetic sequence SARS-CoV-2 S2D22-v1 mAb CDRL3
QSYDSSLSGPEV

SEQ ID NO: 312 - DNA, 388 bp, Artificial Sequence
  Synthetic sequence SARS-CoV-2 S2D22-v1 mAb VH
gaggtgcatctggtggagtccgggggaggcttggtccagcctggggggtccctgaaactctcctgtgcagcctctgggttcaccttcagtggctctgctatgcactgggtccgccaggcttccgggaaagggctggagtgggttggccgtataagaaccaaagctaatacttacgcgacagcatatgctgcgtcggtgaaaggcaggttcaccatctccagagatgattcaaagaacacggcttatctgcaaatgaacagcctgaaaaccgaggacacggccgtgtattactgtactagacccgccccctacgattttttgagtgattattataccggtgaacaacttgactactggggccagggaaccctggtcaccgtctcctcag

SEQ ID NO: 313 - DNA, 337 bp, Artificial Sequence
  Synthetic sequence SARS-CoV-2 S2D22-v1 mAb VL
cagtctgtgctgacgcagccgccctcagtgtctggggccccagggcagagggtcaccatctcctgcactgggagcagctccaacatcggggcaggttatgatgtacactggtaccagcaacttccaggagcagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggtccggaggtcttcggaactgggaccaaggtcaccgtcctgg

SEQ ID NO: 314 - protein, 126 aa, Artificial Sequence
  Synthetic sequence SARS-CoV-2 S2D43-v1 mAb VH
QVTLKESGPTLVKPTQTLTLTCTFSGFSLSTSGVGVGWLRQPPGKALEWLALIYWDDDKRYSPSLKNRLTVTKDTSKNQVVLTMTNLDPVDTATYYCAHDNTLGGSSTWQSTFDYWGQGTLVTVSS

SEQ ID NO: 315 - protein, 10 aa, Artificial Sequence
  Synthetic sequence SARS-CoV-2 S2D43-v1 mAb CDRH1
GFSLSTSGVG

SEQ ID NO: 316 - protein, 7 aa, Artificial Sequence
  Synthetic sequence SARS-CoV-2 S2D43-v1 mAb CDRH2
IYWDDDK

SEQ ID NO: 317 - protein, 18 aa, Artificial Sequence
  Synthetic sequence SARS-CoV-2 S2D43-v1 mAb CDRH3
AHDNTLGGSSTWQSTFDY

SEQ ID NO: 318 - protein, 110 aa, Artificial Sequence
  Synthetic sequence SARS-CoV-2 S2D43-v1 mAb VL
QSVLTQPPSVSEAPRQRVTISCSGSNSNIGNNAVHWYQQLPGKAPKLLIYYDDLLPSGVSDRFSGSKSGTSASLAISGLQSEDEADYYCAAWDDRMNGPVFGGGTKLTVL

SEQ ID NO: 319 - protein, 8 aa, Artificial Sequence
  Synthetic sequence SARS-CoV-2 S2D43-v1 mAb CDRL1
NSNIGNNA

SEQ ID NO: 320 - protein, 3 aa, Artificial Sequence
  Synthetic sequence SARS-CoV-2 S2D43-v1 mAb CDRL2
YDD

SEQ ID NO: 321 - protein, 11 aa, Artificial Sequence
  Synthetic sequence SARS-CoV-2 S2D43-v1 mAb CDRL3
AAWDDRMNGPV

SEQ ID NO: 322 - DNA, 379 bp, Artificial Sequence
  Synthetic sequence SARS-CoV-2 S2D43-v1 mAb VH
caggtcaccttgaaggagtctggtcctacgctggtgaaacccacacagaccctcacgctgacctgcaccttctctgggttctcactcagcactagtggagtgggtgtgggctggctccgtcagcccccaggaaaggccctggagtggcttgcactcatttattgggatgatgataagcgctacagtccatctctgaagaataggctcaccgtcaccaaggacacctccaaaaaccaggtggtcctcacaatgaccaatttggaccctgtggacacagccacatattactgtgcacacgacaacaccttgggaggtagcagcacctggcaatcaacctttgactactggggccagggaaccctggtcaccgtctcctcag

SEQ ID NO: 323 - DNA, 331 bp, Artificial Sequence
  Synthetic sequence SARS-CoV-2 S2D43-v1 mAb VL
cagtctgtgctgactcagccaccctcggtgtctgaagcccccaggcagagggtcaccatctcctgttctggaagcaactccaacatcggaaataatgctgtacactggtaccagcagctcccaggaaaggctcccaaactcctcatctattatgatgatctgctgccctcaggggtctctgaccgattctctggctccaagtctggcacctcagcctccctggccatcagtgggctccagtctgaggatgaggctgattattactgtgcagcatgggatgacaggatgaatggtccggtattcggcggagggaccaagctgaccgtcctag

SEQ ID NO: 324 - protein, 13 aa, Artificial Sequence
  Synthetic sequence SARS-CoV-2 S2D8-v1.1 mAb CDRH3
DLWFREILHGMDV

SEQ ID NO: 325 - protein, 10 aa, Artificial Sequence
  Synthetic sequence SARS-CoV-2 S2D25-v1.1 mAb CDRH1
GFPFNIYAMS

SEQ ID NO: 326 - protein, 17 aa, Artificial Sequence
  Synthetic sequence SARS-CoV-2 S2D25-v1.2 mAb CDRH2
GISGSGGSTYYADSVRG

SEQ ID NO: 327 - protein, 13 aa, Artificial Sequence
  Synthetic sequence SARS-CoV-2 S2D25-v1.3 mAb CDRH3
DLWFREILHGMDV

SEQ ID NO: 328 - protein, 14 aa, Artificial Sequence
  Synthetic sequence SARS-CoV-2 S2D25-v1.4 mAb CDRL1
TGTSSDVGGYNYVS

SEQ ID NO: 329 - protein, 7 aa, Artificial Sequence
  Synthetic sequence SARS-CoV-2 S2D25-v1.5 mAb CDRL2
EVSNRPS

SEQ ID NO: 330 - protein, 10 aa, Artificial Sequence
  Synthetic sequence SARS-CoV-2 S2D32-v1.1 mAb CDRH1
GFTFGDYPMS

SEQ ID NO: 331 - protein, 19 aa, Artificial Sequence
  Synthetic sequence SARS-CoV-2 S2D32-v1.2 mAb CDRH2
FIRSKAYGGTTQYAASVKG

SEQ ID NO: 332 - protein, 17 aa, Artificial Sequence
  Synthetic sequence SARS-CoV-2 S2D32-v1.3 mAb CDRH3
EMWDCSGGRCYSPFFDY

SEQ ID NO: 333 - protein, 11 aa, Artificial Sequence
  Synthetic sequence SARS-CoV-2 S2D32-v1.4 mAb CDRL1
RASQTVSSNLA

SEQ ID NO: 334 - protein, 7 aa, Artificial Sequence
  Synthetic sequence SARS-CoV-2 S2D32-v1.5 mAb CDRL2
GASTRAT

SEQ ID NO: 335 - protein, 10 aa, Artificial Sequence
  Synthetic sequence SARS-CoV-2 S2D60-v1.1 mAb CDRH1
GFTFMSSAVQ

SEQ ID NO: 336 - protein, 17 aa, Artificial Sequence
  Synthetic sequence SARS-CoV-2 S2D60-v1.2 mAb CDRH2
WIVVGSGNTNYTQKFRE

SEQ ID NO: 337 - protein, 14 aa, Artificial Sequence
  Synthetic sequence SARS-CoV-2 S2D60-v1.3 mAb CDRH3
PRCSGGSCHDGFDI

SEQ ID NO: 338 - protein, 12 aa, Artificial Sequence
  Synthetic sequence SARS-CoV-2 S2D60-v1.4 mAb CDRL1
RASQSVSSSYLG

SEQ ID NO: 339 - protein, 7 aa, Artificial Sequence
  Synthetic sequence SARS-CoV-2 S2D60-v1.5 mAb CDRL2
GASSRAT

SEQ ID NO: 340 - protein, 127 aa, Artificial Sequence
  Synthetic sequence SARS-CoV-2 S309-v2 mAb VH
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSS

SEQ ID NO: 341 - protein, 8 aa, Artificial Sequence
  Synthetic sequence SARS-CoV-2 S309-v2 mAb CDRH1
GYPFTSYG

SEQ ID NO: 342 - protein, 8 aa, Artificial Sequence
  Synthetic sequence SARS-CoV-2 S309-v2 mAb CDRH2
ISTYQGNT

SEQ ID NO: 343 - protein, 20 aa, Artificial Sequence
  Synthetic sequence SARS-CoV-2 S309-v2 mAb CDRH3
ARDYTRGAWFGESLIGGFDN

SEQ ID NO: 344 - protein, 107 aa, Artificial Sequence [sequence omitted]

SEQ ID NO: 345 - protein, 7 aa, Artificial Sequence
  Synthetic sequence SARS-CoV-2 S309-v2 mAb CDRL1
QTVSSTS

SEQ ID NO: 346 - protein, 3 aa, Artificial Sequence
  Synthetic sequence SARS-CoV-2 S309-v2 mAb CDRL2
GAS

SEQ ID NO: 347 - protein, 8 aa, Artificial Sequence
  Synthetic sequence SARS-CoV-2 S309-v2 mAb CDRL3
QQHDTSLT

SEQ ID NO: 348 - protein, 121 aa, Artificial Sequence
  Synthetic sequence SARS-CoV-2 S2X127-v1 mAb VH
EVQLVESGGGLIQPGGSLRLSCAASGLTVRSNYMSWVRQAPGKGLEWVSVMYSGGSTFYADSVKGRSTISRDNSKNTLYLQMNSLRAEDTAVYYCARGDIADDYHYGLDVWGQGTTVTVSS

SEQ ID NO: 349 - protein, 8 aa, Artificial Sequence
  Synthetic sequence SARS-CoV-2 S2X127-v1 mAb CDRH1
GLTVRSNY

SEQ ID NO: 350 - protein, 7 aa, Artificial Sequence
  Synthetic sequence SARS-CoV-2 S2X127-v1 mAb CDRH2
MYSGGST

SEQ ID NO: 351 - protein, 15 aa, Artificial Sequence
  Synthetic sequence SARS-CoV-2 S2X127-v1 mAb CDRH3
ARGDIADDYHYGLDV

SEQ ID NO: 352 - protein, 110 aa, Artificial Sequence
  Synthetic sequence SARS-CoV-2 S2X127-v1 mAb VL
QSVLTQPPSVSGAPGQRVTISCTGSTSNIGAGYNVHWYQHFPGTSPKLLIYGNSNRPSGVPDRFSGSKSGTSASLAITGLQAEDEAAYYCQSYDSNLSGVFGGGTKLTVL

SEQ ID NO: 353 - protein, 9 aa, Artificial Sequence
  Synthetic sequence SARS-CoV-2 S2X127-v1 mAb CDRL1
TSNIGAGYN

SEQ ID NO: 354 - protein, 3 aa, Artificial Sequence
  Synthetic sequence SARS-CoV-2 S2X127-v1 mAb CDRL2
GNS

SEQ ID NO: 355 - protein, 10 aa, Artificial Sequence
  Synthetic sequence SARS-CoV-2 S2X127-v1 mAb CDRL3
QSYDSNLSGV

SEQ ID NO: 356 - DNA, 363 bp, Artificial Sequence
  Synthetic sequence SARS-CoV-2 S2X127-v1 mAb VH
gaggtgcagctggtggagtctggaggaggcttgatccagccgggggggtccctgagactctcctgtgcagcctctgggctcaccgtcaggagcaactatatgagctgggtccgccaggctccagggaaggggctggagtgggtctcagttatgtatagcggtggtagtacattctacgcagactccgtgaagggccgatccaccatctccagagacaattccaagaacacgctgtatcttcaaatgaacagcctgagagccgaggacacggccgtgtattactgtgcgagaggtgatatagcagatgactaccactacggtttggacgtctggggccaagggaccacggtcaccgtctcctcg

SEQ ID NO: 357 - DNA, 331 bp, Artificial Sequence
  Synthetic sequence SARS-CoV-2 S2X127-v1 mAb VL
cagtctgtgttgacgcagccgccctcagtgtctggggccccagggcagagggtcaccatctcctgcactgggagcacctccaacatcggggcaggttataatgtacactggtaccagcactttccaggaacatcccccaaactcctcatctatggtaacagcaatcggccctcaggggtccctgaccgattttctggctccaagtctggcacctcagcctccctggccatcactgggctccaggctgaggatgaggctgcttattactgccagtcctatgacagcaacctgagtggagtgttcggcggagggaccaagctgaccgtcctag

SEQ ID NO: 358 - protein, 124 aa, Artificial Sequence
  Synthetic sequence SARS-CoV-2 S2X129-v1 mAb VH
EVQLVQSGAEVKKPGASVKVSCKASGYTFTDYFMHWVRQAPGQGLEWMGWISPNSGGTNYAQRFQGRVTMTRDTSISTTYMELSRLRSDDTAVYYCARDQAYIVLAQGSGMDVWGQGTTVTVSS

SEQ ID NO: 359 - protein, 8 aa, Artificial Sequence
  Synthetic sequence SARS-CoV-2 S2X129-v1 mAb CDRH1
GYTFTDYF

SEQ ID NO: 360 - protein, 8 aa, Artificial Sequence
  Synthetic sequence SARS-CoV-2 S2X129-v1 mAb CDRH2
ISPNSGGT

SEQ ID NO: 361 - protein, 17 aa, Artificial Sequence
  Synthetic sequence SARS-CoV-2 S2X129-v1 mAb CDRH3
ARDQAYIVLAQGSGMDV

SEQ ID NO: 362 - protein, 107 aa, Artificial Sequence
  Synthetic sequence SARS-CoV-2 S2X129-v1 mAb VL(VK)
DIVMTQTPSSVSASVGDRVTITCRASQGISSWLAWYQQKPGKAPKLLISAASSLQSGVPSRFSGSGSGTDFTLTISTLQPEDFATYYCQQANSFPLAFGPGTKVDIK

SEQ ID NO: 363 - protein, 6 aa, Artificial Sequence
  Synthetic sequence SARS-CoV-2 S2X129-v1 mAb CDRL1
QGISSW

SEQ ID NO: 364 - protein, 3 aa, Artificial Sequence
  Synthetic sequence SARS-CoV-2 S2X129-v1 mAb CDRL2
AAS

SEQ ID NO: 365 - protein, 9 aa, Artificial Sequence
  Synthetic sequence SARS-CoV-2 S2X129-v1 mAb CDRL3
QQANSFPLA

SEQ ID NO: 366 - DNA, 372 bp, Artificial Sequence
  Synthetic sequence SARS-CoV-2 S2X129-v1 mAb VH
gaggtgcagctggtgcagtctggggctgaggtgaagaagcctggggcctccgtgaaggtctcctgcaaggcttctggatacaccttcaccgactactttatgcactgggtgcgacaggcccctggacaagggcttgagtggatgggatggatcagccctaacagtggtggcacaaactatgcacagaggtttcagggcagggtcaccatgaccagggacacgtccatcagtacaacctacatggagctgagcaggctgagatctgacgacacggccgtgtattactgtgcgagagatcaggcgtatattgttctagcccaaggctccggtatggacgtctggggccaagggaccacggtcaccgtctcctca

SEQ ID NO: 367 - DNA, 322 bp, Artificial Sequence
  Synthetic sequence SARS-CoV-2 S2X129-v1 mAb VL(VK)
gatattgtgatgacccagactccatcttccgtgtctgcatctgtaggagacagagtcaccatcacttgtcgggcgagtcagggtattagcagctggttagcctggtatcagcagaaaccagggaaagcccctaagctcctgatctctgctgcatccagtttgcaaagtggggtcccatcaaggttcagcggcagtggatctgggacagatttcactctcaccatcagcaccctgcagcctgaagattttgcaacttactattgtcaacaggctaacagtttccccctcgctttcggccctgggaccaaagtggatatcaaac

SEQ ID NO: 368 - protein, 121 aa, Artificial Sequence
  Synthetic sequence SARS-CoV-2 S2X132-v1 mAb VH
EVQLVESGGGLVQPGGSLRLSCAASGFTVSSNYMSWVRQAPGKGLEWVSVIYSGGNTYYADSVKGRFTISRDNSKNTLYLQMISLRAEDTAVYYCARDRRLPSIIFGLDVWGQGTTVTVSS

SEQ ID NO: 369 - protein, 8 aa, Artificial Sequence
  Synthetic sequence SARS-CoV-2 S2X132-v1 mAb CDRH1
GFTVSSNY

SEQ ID NO: 370 - protein, 7 aa, Artificial Sequence
  Synthetic sequence SARS-CoV-2 S2X132-v1 mAb CDRH2
IYSGGNT

SEQ ID NO: 371 - protein, 15 aa, Artificial Sequence
  Synthetic sequence SARS-CoV-2 S2X132-v1 mAb CDRH3
ARDRRLPSIIFGLDV

SEQ ID NO: 372 - protein, 111 aa, Artificial Sequence
  Synthetic sequence SARS-CoV-2 S2X132-v1 mAb VL
QSALTQPASVSGSPGQSITISCTGTSSDVGGNNHVSWYRQHPGKAPKLMIYEVSNRPSGVSNRFSGSKSGNTASLTISGLQAGDEADYYCSSFTTYTTDVVFGGGTKLTVL

SEQ ID NO: 373 - protein, 9 aa, Artificial Sequence
  Synthetic sequence SARS-CoV-2 S2X132-v1 mAb CDRL1
SSDVGGNNH

SEQ ID NO: 374 - protein, 3 aa, Artificial Sequence
  Synthetic sequence SARS-CoV-2 S2X132-v1 mAb CDRL2
EVS

SEQ ID NO: 375 - protein, 11 aa, Artificial Sequence
  Synthetic sequence SARS-CoV-2 S2X132-v1 mAb CDRL3
SSFTTYTTDVV

SEQ ID NO: 376 - DNA, 363 bp, Artificial Sequence
  Synthetic sequence SARS-CoV-2 S2X132-v1 mAb VH
gaggtgcagctggtggagtctggaggaggcttggtccagcctggggggtccctgagactctcctgtgcagcctctggattcaccgtcagtagcaactacatgagctgggtccgccaggctccagggaaggggctggagtgggtctcagttatttatagcggtggtaacacatactacgcagactccgtaaagggccgattcaccatctccagagacaattccaagaacacgctgtatcttcaaatgatcagcctgagagccgaggacacggccgtgtattactgtgcgagagataggaggttgccctcgatcatcttcggtctggacgtctggggccaagggaccacggtcaccgtctcctca

SEQ ID NO: 377 - DNA, 334 bp, Artificial Sequence
  Synthetic sequence SARS-CoV-2 S2X132-v1 mAb VL
cagtctgccctgactcagcctgcctccgtgtctgggtctcctggacagtcgatcaccatctcctgcactggaaccagcagtgacgttggtggtaataaccatgtctcctggtaccgacagcacccaggcaaagcccccaaactcatgatttatgaggtcagtaatcggccctcaggggtttctaatcgcttctctggctccaagtctggcaacacggcctccctgaccatctctgggctccaggctggggacgaggctgattattactgcagctcatttacaacctacaccacggatgtggtattcggcggagggaccaagcttaccgtcctag

SEQ ID NO: 378 - protein, 122 aa, Artificial Sequence
  Synthetic sequence SARS-CoV-2 S2X190-v1 mAb VH
EVQLVESGGGVVQPGRSLRLSCAASGFPFSTYGMHWVRQAPGKGLEWVAVIWYDGSTKYYADSVKGRFTISRDNSKNTLYLQMNSLRAEDTALYYCARVHSSGPGDEYFQHWGQGTLLTVSS

SEQ ID NO: 379 - protein, 8 aa, Artificial Sequence
  Synthetic sequence SARS-CoV-2 S2X190-v1 mAb CDRH1
GFPFSTYG

SEQ ID NO: 380 - protein, 8 aa, Artificial Sequence
  Synthetic sequence SARS-CoV-2 S2X190-v1 mAb CDRH2
IWYDGSTK

SEQ ID NO: 381 - protein, 15 aa, Artificial Sequence
  Synthetic sequence SARS-CoV-2 S2X190-v1 mAb CDRH3
ARVHSSGPGDEYFQH

SEQ ID NO: 382 - protein, 107 aa, Artificial Sequence
  Synthetic sequence SARS-CoV-2 S2X190-v1 mAb VL(VK)
DIVMTQSPSSLSASVGDRVTITCRASQGISSGLAWYQQKPGKGPKVLIYDASSLESGVPSRFSGSASGTDFTLTISSLQPEDFATYFCQQFYSYPLTFGGGTKVEIK

SEQ ID NO: 383 - protein, 6 aa, Artificial Sequence
  Synthetic sequence SARS-CoV-2 S2X190-v1 mAb CDRL1
QGISSG

SEQ ID NO: 384 - protein, 3 aa, Artificial Sequence
  Synthetic sequence SARS-CoV-2 S2X190-v1 mAb CDRL2
DAS

SEQ ID NO: 385 - protein, 9 aa, Artificial Sequence
  Synthetic sequence SARS-CoV-2 S2X190-v1 mAb CDRL3
QQFYSYPLT

SEQ ID NO: 386 - DNA, 367 bp, Artificial Sequence
  Synthetic sequence SARS-CoV-2 S2X190-v1 mAb VH
gaggtgcagctggtggagtctgggggaggcgtggtccagcctggcaggtccctgagactctcctgtgcagcgtctggattccccttcagtacctatggcatgcactgggtccgccaggctccaggcaaggggctggagtgggtggcagttatatggtatgatggaagtactaaatactatgcagactccgtgaagggccgattcaccatctccagagacaattccaagaacactctctatctgcaaatgaacagcctcagagccgaggacacggctttgtattactgtgcgagagtccatagcagtggcccgggggatgaatacttccagcactggggccagggcaccctgctcaccgtctcctcag

SEQ ID NO: 387 - DNA, 322 bp, Artificial Sequence
  Synthetic sequence SARS-CoV-2 S2X190-v1 mAb VL(VK)
gacatcgtgatgacccagtctccatcctccctgtctgcatctgtaggagacagagtcaccatcacttgccgggcaagtcagggcatcagcagtggtttagcctggtatcaacagaaaccagggaaaggtcctaaggtcctgatctatgatgcctccagtttggaaagtggagtcccatcaaggttcagcggcagtgcatctgggacagatttcactctcaccatcagcagcctgcagcctgaagattttgcaacttatttctgtcaacagttttatagttaccctctcactttcggcggagggaccaaggtggagatcaagc

SEQ ID NO: 388 - protein, 117 aa, Artificial Sequence
  Synthetic sequence SARS-CoV-2 S2X200-v1 mAb VH
EVHLVESGGGLIQPGGSLRLSCAASGITVSSNYMSWVRQAPGKGLEWVSVIYSGGSTFYADSVKGRFTISRDNSKNTVYLQMNSLRAEDTAVYYCARDLVTYGMDVWGQGTTVTVSS

SEQ ID NO: 389 - protein, 8 aa, Artificial Sequence
  Synthetic sequence SARS-CoV-2 S2X200-v1 mAb CDRH1
GITVSSNY

SEQ ID NO: 390 - protein, 7 aa, Artificial Sequence
  Synthetic sequence SARS-CoV-2 S2X200-v1 mAb CDRH2
IYSGGST

SEQ ID NO: 391 - protein, 11 aa, Artificial Sequence
  Synthetic sequence SARS-CoV-2 S2X200-v1 mAb CDRH3
ARDLVTYGMDV

SEQ ID NO: 392 - protein, 107 aa, Artificial Sequence
  Synthetic sequence SARS-CoV-2 S2X200-v1 mAb VL(VK)
EIVLTQSPSFLSASVGDRVTITCRASQGISSYLAWYQQKPGKAPKLLIYAASTLQSGVPSRFSGSGSGTEFTLTISSLQPEDFATYYCQQLNGDPPIFGHGTRLEIK

SEQ ID NO: 393 - protein, 6 aa, Artificial Sequence
  Synthetic sequence SARS-CoV-2 S2X200-v1 mAb CDRL1
QGISSY

SEQ ID NO: 394 - protein, 3 aa, Artificial Sequence
  Synthetic sequence SARS-CoV-2 S2X200-v1 mAb CDRL2
AAS

SEQ ID NO: 395 - protein, 9 aa, Artificial Sequence
  Synthetic sequence SARS-CoV-2 S2X200-v1 mAb CDRL3
QQLNGDPPI

SEQ ID NO: 396 - DNA, 351 bp, Artificial Sequence
  Synthetic sequence SARS-CoV-2 S2X200-v1 mAb VH
gaggtgcatctggtggagtctggaggaggcttgatccagcctggggggtccctgagactctcctgtgcagcctctgggatcaccgtcagtagcaactatatgagctgggtccgccaggctccagggaaggggctggagtgggtctcagttatttatagcggtggtagcacattctatgcagactccgtgaagggccgattcaccatctccagagacaattccaagaacacggtgtatcttcaaatgaacagcctgagagccgaggacacggccgtgtattactgtgcgagagatctggtaacctacggtatggacgtctggggccaagggaccacggtcaccgtctcctca

SEQ ID NO: 397 - DNA, 322 bp, Artificial Sequence
  Synthetic sequence SARS-CoV-2 S2X200-v1 mAb VL(VK)
gaaattgtgttgacacagtctccatccttcctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatggtgaccctcctatcttcggccatgggacacgactggagattaaac

SEQ ID NO: 398 - protein, 124 aa, Artificial Sequence
  Synthetic sequence SARS-CoV-2 S2X227-v1 mAb VH
EVQLVQSGAEVKKPGASVKVSCKASGYTFTSYYMHWVRQAPGQGLEWMGIINPGGVSTTYAHYAQKFQGRVTMTRDTSTSTVYMELSSLRSEDTAVYYCARSIAVFWGDAFDIWGQGTMVTVSS

SEQ ID NO: 399 - protein, 8 aa, Artificial Sequence
  Synthetic sequence SARS-CoV-2 S2X227-v1 mAb CDRH1
GYTFTSYY

SEQ ID NO: 400 - protein, 8 aa, Artificial Sequence
  Synthetic sequence SARS-CoV-2 S2X227-v1 mAb CDRH2
INPGGVST

SEQ ID NO: 401 - protein, 14 aa, Artificial Sequence
  Synthetic sequence SARS-CoV-2 S2X227-v1 mAb CDRH3
ARSIAVFWGDAFDI

SEQ ID NO: 402 - protein, 113 aa, Artificial Sequence
  Synthetic sequence SARS-CoV-2 S2X227-v1 mAb VL(VK)
DIQMTQSPDSLAVSLGERATINCKSSQSVLYSSNNKNYLAWYQQKPGQPPKLLIYWASTRESGVPDRFSGSGSGTDFTLTISSLQAEDVAVYYCQQYSSSPLTFGGGTKVEIK

SEQ ID NO: 403 - protein, 12 aa, Artificial Sequence
  Synthetic sequence SARS-CoV-2 S2X227-v1 mAb CDRL1
QSVLYSSNNKNY

SEQ ID NO: 404 - protein, 3 aa, Artificial Sequence
  Synthetic sequence SARS-CoV-2 S2X227-v1 mAb CDRL2
WAS

SEQ ID NO: 405 - protein, 9 aa, Artificial Sequence
  Synthetic sequence SARS-CoV-2 S2X227-v1 mAb CDRL3
QQYSSSPLT

SEQ ID NO: 406 - DNA, 373 bp, Artificial Sequence
  Synthetic sequence SARS-CoV-2 S2X227-v1 mAb VH
gaggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctggtggtgttagcacaacgtacgcacattacgcacagaagttccagggcagagtcaccatgaccagggacacgtccacgagcacagtctacatggagctgagcagcctgagatctgaggacacggccgtgtattactgtgcgagatctatagcagtgttttggggagatgcttttgatatctggggccaagggacaatggtcaccgtctcttcag

SEQ ID NO: 407 - DNA, 340 bp, Artificial Sequence
  Synthetic sequence SARS-CoV-2 S2X227-v1 mAb VL(VK)
gacatccagatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcgtctacccgggaatccggggtccctgaccgattcagtggcagcgggtctgggacagatttcactctcaccatcagcagcctgcaggctgaagatgtggcagtttattactgtcagcaatattctagttctcccctcactttcggcggagggaccaaggtggagatcaaac

SEQ ID NO: 408 - protein, 126 aa, Artificial Sequence
  Synthetic sequence SARS-CoV-2 S2X259-v1 mAb VH
QVQLVQSGAEVKKPGSSVKVSCKASGGIFNTYTISWVRQAPGQGLEWMGRIILMSGMANYAQKIQGRVTITADKSTSTAYMELTSLRSDDTAVYYCARGFNGNYYGWGDDDAFDISGQGTLVTVYS

SEQ ID NO: 409 - protein, 8 aa, Artificial Sequence
  Synthetic sequence SARS-CoV-2 S2X259-v1 mAb CDRH1
GGIFNTYT

SEQ ID NO: 410 - protein, 8 aa, Artificial Sequence
  Synthetic sequence SARS-CoV-2 S2X259-v1 mAb CDRH2
IILMSGMA

SEQ ID NO: 411 - protein, 19 aa, Artificial Sequence
  Synthetic sequence SARS-CoV-2 S2X259-v1 mAb CDRH3
ARGFNGNYYGWGDDDAFDI

SEQ ID NO: 412 - protein, 113 aa, Artificial Sequence
  Synthetic sequence SARS-CoV-2 S2X259-v1 mAb VL
QTVLTQPPSVSGAPGQRVTISCTGSNSNIGAGYDVHWYQQLPGTAPKLLICGNSNRPSGVPDRFSGSKSGTSASLAITGLQAEDEADYYCQSYDSSLSGPNWVFGGGTKLTVL

SEQ ID NO: 413 - protein, 9 aa, Artificial Sequence
  Synthetic sequence SARS-CoV-2 S2X259-v1 mAb CDRL1
NSNIGAGYD

SEQ ID NO: 414 - protein, 3 aa, Artificial Sequence
  Synthetic sequence SARS-CoV-2 S2X259-v1 mAb CDRL2
GNS

SEQ ID NO: 415 - protein, 13 aa, Artificial Sequence
  Synthetic sequence SARS-CoV-2 S2X259-v1 mAb CDRL3
QSYDSSLSGPNWV

SEQ ID NO: 416 - DNA, 379 bp, Artificial Sequence
  Synthetic sequence SARS-CoV-2 S2X259-v1 mAb VH
caggtacagctggtgcaatctggggctgaggtgaagaagcctgggtcctcggtgaaggtctcctgcaaggcttctgggggcatcttcaacacctatactatcagctgggtgcgacaggcccctgggcaagggcttgagtggatgggaaggatcatccttatgtctggtatggcaaactacgcacagaagatccagggcagagtcacgataaccgcggacaaatcgacgagcacagcctacatggagctgaccagcctgagatctgacgacacggccgtctattactgtgcgagaggcttcaacgggaactattatggttggggggacgatgatgcttttgatatctcgggccaagggacactggtcaccgtctattcag

SEQ ID NO: 417 - DNA, 340 bp, Artificial Sequence
  Synthetic sequence SARS-CoV-2 S2X259-v1 mAb VL
cagactgtgttgacgcagccgccctcagtgtctggggccccagggcagagggtcaccatctcctgcactgggagcaactccaacatcggggctggttatgatgtacactggtaccagcagcttccaggaacagcccccaaactcctcatctgtggtaacagcaatcggccctcaggggtccctgaccgattctctggctccaagtctggcacctcagcctccctggccatcactgggctccaggctgaggatgaggctgattattactgccagtcctatgacagcagcctgagtggcccgaattgggtgttcggcggagggaccaagctgaccgtcctac

SEQ ID NO: 418 - protein, 124 aa, Artificial Sequence
  Synthetic sequence SARS-CoV-2 S2X288-v1 mAb VH
EVQLVESGGGLIQPGGSLRLSCAASGFTVSSNYMSWVRQAPGKGLEWVSVIYFGGTTYYADSVKGRFTISRDTSENTLFLQMNSLRVEDTAVYYCARDQGIAVAGLDFGAYDIWGQGTMVTVSS

SEQ ID NO: 419 - protein, 8 aa, Artificial Sequence
  Synthetic sequence SARS-CoV-2 S2X288-v1 mAb CDRH1
GFTVSSNY

SEQ ID NO: 420 - protein, 7 aa, Artificial Sequence
  Synthetic sequence SARS-CoV-2 S2X288-v1 mAb CDRH2
IYFGGTT

SEQ ID NO: 421 - protein, 18 aa, Artificial Sequence
  Synthetic sequence SARS-CoV-2 S2X288-v1 mAb CDRH3
ARDQGIAVAGLDFGAYDI

SEQ ID NO: 422 - protein, 114 aa, Artificial Sequence
  Synthetic sequence SARS-CoV-2 S2X288-v1 mAb VL(VK)
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYRTPTWTFGQGTKVEIK

SEQ ID NO: 423 - protein, 12 aa, Artificial Sequence
  Synthetic sequence SARS-CoV-2 S2X288-v1 mAb CDRL1
QSVLYSSNNKNY

SEQ ID NO: 424 - protein, 3 aa, Artificial Sequence
  Synthetic sequence SARS-CoV-2 S2X288-v1 mAb CDRL2
WAS

SEQ ID NO: 425 - protein, 10 aa, Artificial Sequence
  Synthetic sequence SARS-CoV-2 S2X288-v1 mAb CDRL3
QQYYRTPTWT

SEQ ID NO: 426 - DNA, 373 bp, Artificial Sequence
  Synthetic sequence SARS-CoV-2 S2X288-v1 mAb VH
gaggtgcagctggtggagtctggaggaggcttgatccagcctggggggtccctgagactctcctgtgcagcctctgggttcaccgtcagtagcaactacatgagctgggtccgccaggctccagggaaggggctggagtgggtctcagttatttatttcggtggtaccacatactacgcagactccgtgaagggccgattcaccatctccagagacacttccgagaacacgctgtttcttcagatgaacagcctgagagtcgaggacacggccgtgtattactgtgccagagatcagggtatagcagtggctggtctcgattttggcgcttatgatatctggggccaagggacaatggtcaccgtctcttcag

SEQ ID NO: 427 - DNA, 343 bp, Artificial Sequence
  Synthetic sequence SARS-CoV-2 S2X288-v1 mAb VL(VK)
gacatcgtgatgacccagtctccagactccctggctgtgtctctgggcgagagggccaccatcaactgcaagtccagccagagtgttttatatagctccaacaataagaactacttagcttggtaccagcagaagccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattataggactcccacgtggacgttcggccaagggaccaaggtggaaatcaaac

SEQ ID NO: 428 - protein, 126 aa, Artificial Sequence
  Synthetic sequence Antibody 407_10_1_v2 (VH)
QVQLVQSGAEVKKPGSSVKVSCKASGGIFNTYTISWVRQAPGQGLEWMGRIILMSGMANYAQKIQGRVTITADKSTSTAYMELTSLRSDDTAVYYCARGFNGNYYGWGDDDAFDIWGQGTLVTVYS

SEQ ID NO: 429 - DNA, 378 bp, Artificial Sequence
  Synthetic sequence Antibody 407_10_1_v2 (VH)
caggtgcagctggtccagagcggcgcagaggtcaaaaagcccggcagtagtgtcaaggtgtcttgtaaagcatcagggggtatcttcaacacctacacaatcagctgggtgagacaggctccaggacagggactggagtggatgggccgcatcatcctgatgtctggcatggccaattacgctcagaagatccagggcagggtgaccatcacagccgacaagtccaccagcacagcttatatggagctgacctctctgaggtccgacgatacagccgtgtactattgcgctcggggcttcaacggcaattactatggctggggagatgatgatgcttttgacatttgggggcagggcactctggtgacagtctacagt

SEQ ID NO: 430 - DNA, 372 bp, Artificial Sequence
  Synthetic sequence SARS-CoV-2 S2X227-v1 (WT)
gaagtgcaactagtgcaaagtggtgcagaagtcaagaagcccggcgcttctgttaaagtgtcctgcaaggcctctggctacacctttacatcctactacatgcactgggtgcggcaggctcctggccagggcctggagtggatgggcatcatcaaccctggaggagtgagcaccacctacgctcactacgcccagaagttccagggcagagtgacaatgacccgggacacctccacctctaccgtgtacatggaactgtcctctctgagatctgaggataccgctgtgtactattgtgccagatccatcgccgtgttctggggcgacgccttcgacatctggggccaaggcaccatggtgaccgtgtccagc

SEQ ID NO: 431 - DNA, 339 bp, Artificial Sequence
  Synthetic sequence SARS-CoV-2 S2X227-v1 mAb VL(VK)
gatatccaaatgactcaaagtccagatagtctcgctgtgtccctgggcgagcgggctaccatcaactgcaagtccagccagtccgtgctgtactcctccaataacaagaactacctggcctggtatcaacagaagcctggccagcctccaaagctgctgatctactgggcctctaccagagagtccggcgtccccgatagattctccggatctggctctggcaccgacttcaccctgaccatctcctctctgcaggccgaggacgtggccgtgtactactgtcagcagtacagctcttctcctctgacctttggcggcggaacaaaagtggaaatcaag

SEQ ID NO: 432 - protein, 124 aa, Artificial Sequence
  Synthetic sequence Antibody 407_10_2_v2 VH
EVQLVQSGAEVKKPGASVKVSCKASGYTFTSYYIHWVRQAPGQGLEWMGIINPGGVSTTYAHYAQKFQGRVTMTRDTSTSTVYMELSSLRSEDTAVYYCARSIAVFWGDAFDIWGQGTMVTVSS

SEQ ID NO: 433 - DNA, 372 bp, Artificial Sequence
  Synthetic sequence Antibody  407_10_2_v2 VH
gaagtgcaactagtgcaaagtggtgcagaagtcaagaagcccggagcttctgtgaaagtgtcctgcaaggcctctggctacacctttacctcctactacatccactgggtgcggcaggctcctggacaaggcctggagtggatgggcatcatcaaccctggcggcgtgtccaccacctacgctcactacgcccagaagttccagggcagagtgacaatgaccagagataccagcacatctaccgtgtacatggaactgtcctctctgcggtccgaggacaccgctgtgtactattgtgccagatccatcgccgtgttctggggcgacgccttcgacatctggggccagggcaccatggttaccgtgtctagc

SEQ ID NO: 434 - protein, 124 aa, Artificial Sequence
  Synthetic sequence Antibody  407_10_2_v3 VH
EVQLVQSGAEVKKPGASVKVSCKASGYTFTSYYMHWVRQAPGQGLEWMGIINPGGVSTTYAHYAQKFQGRVTMTRDTSTSTVYMELSSLRSEDTAVYYCARSIAVFFGDAFDIWGQGTMVTVSS

SEQ ID NO: 435 - protein, 14 aa, Artificial Sequence
  Synthetic sequence Antibody  407_10_2_v3 CDRH3
ARSIAVFFGDAFDI

SEQ ID NO: 436 - DNA, 372 bp, Artificial Sequence
  Synthetic sequence Antibody  407_10_2_v3 VH
gaagtgcaactagtgcaaagtggtgcagaagtcaagaagcccggagcttctgtgaaagtgtcctgcaaggcctctggctacacctttacctcctactacatgcactgggtccggcaggctcctggacaaggcctggagtggatgggcatcatcaaccctggcggcgtgtctaccacctacgctcactacgcccagaagttccagggcagagtgacaatgaccagagataccagcacctctacagtgtacatggaactgtcctctctgcggtccgaggacaccgctgtgtactattgtgccagatccatcgccgtgttcttcggcgacgccttcgacatctggggccagggcaccatggtgaccgtgtccagc

SEQ ID NO: 437 - DNA, 372 bp, Artificial Sequence
  Synthetic sequence Antibody  407_10_2_v4 VH
gaagtgcaactagtgcaaagtggtgcagaagtgaagaagcctggcgcttctgttaaagtgtcctgcaaggcctctggctacacctttacctcctactacatccactgggtgcggcaggctcctggacaaggcctggagtggatgggcatcatcaaccccggcggcgtgtctaccacctacgctcactacgcccagaagttccagggaagagtgaccatgaccagagataccagcacatctacagtgtacatggaactgtcctctctgcggtccgaggacaccgctgtgtactattgtgccagatccatcgccgtgttcttcggcgacgccttcgacatctggggccagggcaccatggtcaccgtgtccagc

SEQ ID NO: 438 - DNA, 372 bp, Artificial Sequence; identical to SEQ ID NO: 437
  Synthetic sequence Antibody  407_10_2_v4 VH

SEQ ID NO: 439 - protein, 113 aa, Artificial Sequence
  Synthetic sequence Antibody  407_10_2_v5 VL
DIQMTQSPDSLAVSLGERATINCKSSQSVLYSSNNKNYLAWYQQKPGQPPKLLIYFASTRESGVPDRFSGSGSGTDFTLTISSLQAEDVAVYYCQQYSSSPLTFGGGTKVEIK

SEQ ID NO: 440 - protein, 3 aa, Artificial Sequence
  Synthetic sequence Antibody  407_10_2_v5 CDRL2
FAS

SEQ ID NO: 441 - DNA, 339 bp, Artificial Sequence
  Synthetic sequence Antibody  407_10_2_v5 VL
gatatccaaatgactcaaagtccagatagtctcgctgtgtccctgggcgagagagctacaatcaactgcaagtccagccagtccgtgctgtactcctccaataacaagaactacctggcctggtaccagcagaaaccaggccagcctcctaagctgctgatctacttcgcctccaccagagagtccggcgtccccgatcggttttctggctctggatctggcaccgacttcaccctgaccatctcctctctgcaggccgaggacgtggccgtgtactactgtcagcaatattctagctctcctctgaccttcggcggcggaaccaaggtggaaatcaag

SEQ ID NO: 442 - protein, 113 aa, Artificial Sequence
  Synthetic sequence Antibody S2X259-v3 VL
QTVLTQPPSVSGAPGQRVTISCTGSNSNIGAGYDVHWYQQLPGTAPKLLISGNSNRPSGVPDRFSGSKSGTSASLAITGLQAEDEADYYCQSYDSSLSGPNWVFGGGTKLTVL

SEQ ID NO: 443 - protein, 113 aa, Artificial Sequence
  Synthetic sequence Antibody S2X259-v4 VL
QTVLTQPPSVSGAPGQRVTISCTGSNSNIGAGYDVHWYQQLPGTAPKLLIAGNSNRPSGVPDRFSGSKSGTSASLAITGLQAEDEADYYCQSYDSSLSGPNWVFGGGTKLTVL

SEQ ID NO: 444 - protein, 113 aa, Artificial Sequence
  Synthetic sequence Antibody S2X259-v5 VL
QTVLTQPPSVSGAPGQRVTISCTGSNSNIGAGYDVHWYQQLPGTAPKLLIYGNSNRPSGVPDRFSGSKSGTSASLAITGLQAEDEADYYCQSYDSSLSGPNWVFGGGTKLTVL

SEQ ID NO: 445 - protein, 113 aa, Artificial Sequence
  Synthetic sequence Antibody S2X259-v6 VL
QTVLTQPPSVSGAPGQRVTISCTGSNSNIGAGYDVHWYQQLPGTAPKLLIVGNSNRPSGVPDRFSGSKSGTSASLAITGLQAEDEADYYCQSYDSSLSGPNWVFGGGTKLTVL

SEQ ID NO: 446 - protein, 126 aa, Artificial Sequence
  Synthetic sequence Antibody S2X259-v7 VH
QVQLVQSGAEVKKPGSSVKVSCKASGGIFNTYTISWVRQAPGQGLEWMGRIILISGIANYAQKIQGRVTITADKSTSTAYMELTSLRSDDTAVYYCARGFNGNYYGWGDDDAFDISGQGTLVTVYS

SEQ ID NO: 447 - protein, 8 aa, Artificial Sequence
  Synthetic sequence Antibody S2X259-v7 CDRH2
IILISGIA

SEQ ID NO: 448 - protein, 126 aa, Artificial Sequence
  Synthetic sequence Antibody S2X259-v8 VH
QVQLVQSGAEVKKPGSSVKVSCKASGGIFSTYTISWVRQAPGQGLEWMGRIILMSGMANYAQKIQGRVTITADKSTSTAYMELTSLRSDDTAVYYCARGFNGNYYGWGDDDAFDISGQGTLVTVYS

SEQ ID NO: 449 - protein, 8 aa, Artificial Sequence
  Synthetic sequence Antibody S2X259-v8 CDRH1
GGIFSTYT

SEQ ID NO: 450 - protein, 1255 aa, Artificial Sequence
  Synthetic sequence SARS-CoV; Urbani strain; surface
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 451 - DNA, 767 bp, Artificial Sequence
  Synthetic sequence CMV promoter
gacattgattattgactagttattaatagtaatcaattacggggtcattagttcatagcccatatatggagttccgcgttacataacttacggtaaatggcccgcctggctgaccgcccaacgacccccgcccatgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttag

SEQ ID NO: 452 - DNA, 50 bp, Artificial Sequence
  Signal peptide
atgggatggtcatgtatcatcctttttctagtagcaactgcaaccggtgt

SEQ ID NO: 453 - DNA, 135 bp, Artificial Sequence
  Synthetic sequence Poly-adenylation signal sequence
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 454 - protein, 16 aa, Artificial Sequence
  Signal peptide
MGWSCIILFLVATATG

SEQ ID NO: 455 - DNA, 57 bp, Artificial Sequence
  Signal peptide
atgggctggtcctgcatcatcctgttcctggtggccacagccaccggcgtgcacagc

SEQ ID NO: 456 - protein, 19 aa, Artificial Sequence
  Signal peptide
MGWSCIILFLVATATGVHS

SEQ ID NO: 457 - protein, 8 aa, Artificial Sequence
  Synthetic sequence S2X259 variant CDRH2
IILMSGKA

SEQ ID NO: 458 - protein, 126 aa, Artificial Sequence
  Synthetic sequence S2X259 variant VH W118_S127
QVQLVQSGAEVKKPGSSVKVSCKASGGIFNTYTISWVRQAPGQGLEWMGRIILMSGMANYAQKIQGRVTITADKSTSTAYMELTSLRSDDTAVYYCARGFNGNYYGWGDDDAFDIWGQGTLVTVSS

SEQ ID NO: 459 - protein, 126 aa, Artificial Sequence
  Synthetic sequence S2X259 variant VH M64K_W118_S127
QVQLVQSGAEVKKPGSSVKVSCKASGGIFNTYTISWVRQAPGQGLEWMGRIILMSGKANYAQKIQGRVTITADKSTSTAYMELTSLRSDDTAVYYCARGFNGNYYGWGDDDAFDIWGQGTLVTVSS

SEQ ID NO: 460 - protein, 126 aa, Artificial Sequence
  Synthetic sequence S2X259 variant VH M59I_M64I_W118_S127
QVQLVQSGAEVKKPGSSVKVSCKASGGIFNTYTISWVRQAPGQGLEWMGRIILISGIANYAQKIQGRVTITADKSTSTAYMELTSLRSDDTAVYYCARGFNGNYYGWGDDDAFDIWGQGTLVTVSS

SEQ ID NO: 461 - DNA, 21 bp, Artificial Sequence
  ACE2 forward primer
caagagcaaacggttgaacac

SEQ ID NO: 462 - DNA, 22 bp, Artificial Sequence
  ACE2 reverse primer
ccagagcctctcattgtagtct

SEQ ID NO: 463 - DNA, 19 bp, Artificial Sequence
  HPRT forward primer
cctggcgtcgtgattagtg

SEQ ID NO: 464 - DNA, 21 bp, Artificial Sequence
  HPRT reverse primer
acaccctttccaaatcctcag

SEQ ID NO: 465 - DNA, 21 bp, Artificial Sequence
  TMPRSS2 forward primer
caagtgctccractctgggat

SEQ ID NO: 466 - DNA, 21 bp, Artificial Sequence
  TMPRSS2 reverse primer
aacacaccgrttctcgtcctc

(19) United States
(12) Patent Application Publication
(10) Pub. No.: US 2024/0059757 A1
(43) Pub. Date: Feb. 22, 2024

(54) ANTIBODIES AGAINST SARS-COV-2 AND METHODS OF USING THE SAME

(71) Applicant: Vir Biotechnology, Inc., San Francisco, CA (US)

(72) Inventors: Martina BELTRAMELLO, Bellinzona (CH); Siro BIANCHI, Bellinzona (CH); Elisabetta CAMERONI, Bellinzona (CH); Davide CORTI, Bellinzona (CH); Nadine CZUDNOCHOWSKI, San Francisco, CA (US); Katja FINK, Bellinzona (CH); Florian A. LEMPP, San Francisco, CA (US); Dora PINTO, Bellinzona (CH); Matteo Samuele PIZZUTO, Bellinzona (CH); Gyorgy SNELL, San Francisco, CA (US); Amalio TELENTI, San Francisco, CA (US); Fabrizia ZATTA, Bellinzona (CH)

(21) Appl. No.: 17/996,209

(22) PCT Filed: Apr. 14, 2021
(86) PCT No.: PCT/US2021/027375

§ 371 Date: Oct. 13, 2022

Related U.S. Application Data
(60) Provisional application No. 63/010,025, filed on Apr. 14, 2020.
(60) Provisional application No. 63/015,399, filed on Apr. 24, 2020.
(60) Provisional application No. 63/019,926, filed on May 4, 2020.
(60) Provisional application No. 63/021,063, filed on May 6, 2020.
(60) Provisional application No. 63/023,808, filed on May 12, 2020.
(60) Provisional application No. 63/030,254, filed on May 26, 2020.
(60) Provisional application No. 63/036,631, filed on Jun. 9, 2020.
(60) Provisional application No. 63/046,452, filed on Jun. 30, 2020.
(60) Provisional application No. 63/057,557, filed on Jul. 28, 2020.
(60) Provisional application No. 63/091,841, filed on Oct. 14, 2020.
(60) Provisional application No. 63/166,879, filed on Mar. 26, 2021.
(60) Provisional application No. 63/170,360, filed on Apr. 2, 2021.
(60) Provisional application No. 63/171,892, filed on Apr. 7, 2021.

(51) Int. Cl. C07K 16/10; G01N 33/569; A61P 31/14

(52) U.S. Cl. CPC C07K 16/1003; G01N 33/56983; A61P 31/14; C07K 2317/76; C07K 2317/52; C07K 2317/92; G01N 2333/165; A61K 2039/505

(57) ABSTRACT

The instant disclosure provides antibodies and antigen-binding fragments thereof that can bind to a SARS-CoV-2 antigen and, in certain embodiments, are capable of neutralizing a SARS-CoV-2 infection. In certain embodiments, the presently disclosed antibodies are capable of binding to S proteins of multiple sarbecoviruses and/or neutralizing infection by multiple sarbecoviruses. Also provided are polynucleotides that encode an antibody or antigen-binding fragment, vectors and host cells that comprise a polynucleotide, pharmaceutical compositions, and methods of using the presently disclosed antibodies, antigen-binding fragments, polynucleotides, vectors, host cells, and compositions to treat or diagnose a sarbecovirus infection, such as a SARS-CoV-2 infection.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 930585_407WO_SEQUENCE_LISTING.txt. The text file is 346 KB, was created on Apr. 14, 2021, and is being submitted electronically via EFS-Web.

### BACKGROUND

A novel betacoronavirus emerged in Wuhan, China, in late 2019. As of Apr. 7, 2021, approximately 132 million cases of infection by this virus (termed, among other names, SARS-CoV-2, and originally identified as Wuhan coronavirus), were confirmed worldwide, and had resulted in approximately 2.87 million deaths. Modalities for preventing or treating SARS-CoV-2 infection, and diagnostic tools for diagnosing a SARS-CoV-2 infection, are needed.

### BRIEF DESCRIPTION OF THE DRAWINGS

FIGS. 1A-IC show results from a neutralization of infection assay using antibodies against SARS-CoV-2 pseudotyped virus. Human monoclonal antibodies isolated from patients recovered from either COVID-19 or SARS infections were expressed recombinantly and were tested in neutralization assays against murine leukemia virus (MLV) pseudotyped with SARS-CoV-2 Spike protein. FIG. 1A shows results for six antibodies isolated from patients recovered from COVID-19. FIG. 1B shows results for six further antibodies isolated from patients recovered from COVID-19 and two antibodies isolated from patients recovered from SARS (S307 and S309 (S309 has the VH of SEQ ID NO.:172 (HCDRs of SEQ ID NOs.:173-175) and the VL SEQ ID NO.:176 (LCDRs of SEQ ID NOs.:177-179), and is described in Pinto et al. Nature 583:290-295 (2020)). FIG. 1C shows results for four of the six antibodies isolated from patients recovered from COVID-19 shown in FIG. 1B. Antibodies were tested at concentrations indicated in the x-axis. Symbols show means of duplicates.

FIGS. 2A-2D show binding of certain antibodies to SARS-CoV-2 Spike protein RBD and SARS-CoV Spike protein RBD. Human monoclonal antibodies were expressed recombinantly and were tested by ELISA. FIG. 2A shows binding of eight antibodies to SARS-CoV-2 Spike protein RBD (left panel) and SARS-CoV Spike protein RBD (right panel). FIG. 2B shows binding of two further antibodies to SARS-CoV-2 Spike protein RBD (left panel) and SARS-CoV Spike protein RBD (right panel). FIG. 2C shows binding of three further antibodies to SARS-CoV-2 Spike protein RBD (left panel) and SARS-CoV Spike protein RBD (right panel). FIG. 2D shows binding of one further antibody to SARS-CoV-2 Spike protein RBD (left panel) and SARS-CoV Spike protein RBD (right panel).

FIGS. 3A-3C show binding of antibodies to SARS-CoV-2 RBD, SARS-CoV-1 RBD, and SARS-CoV-2 S1 domain, as measured by ELISA. FIG. 3A shows binding of recombinant monoclonal antibody S2H7. FIG. 3B shows binding of recombinant monoclonal antibody S2R7. FIG. 3C shows binding of recombinant monoclonal antibody S2R5. Symbols are means of duplicates.

FIGS. 4A and 4B show binding of certain antibodies to SARS-CoV-2 Spike protein ectodomain. Binding to the stabilized prefusion trimer of Spike was measured by ELISA. Recombinant antibodies were diluted 1:3 in the concentration range indicated in the x-axis. EC50 values in ng/ml are given in boxes in the upper right. FIG. 4A shows binding of recombinant monoclonal antibodies S2A15-v1 and S2A15-v2. FIG. 4B shows binding of recombinant monoclonal antibodies S2B2-v1 and S2B2-v2.

FIGS. 5A-5C show competition of certain antibodies for binding to SARS-CoV-2 RBD. HIS-tagged SARS-CoV-2 RBD (residues 331-550 of Spike protein from BetaCoV/Wuhan-Hu-1/2019, accession number MN908947) was loaded onto Octet pins, followed by incubation with a first antibody, followed by incubation with a second antibody. FIG. 5A shows competition of certain antibodies with first antibody S2A5. FIG. 5B shows competition of certain antibodies with first antibody S2A10. FIG. 5C shows competition of certain antibodies with first antibody S309 (VH SEQ ID NO.:172; VL SEQ ID NO.:176; Pinto et al. Nature 583:290-295 (2020)).

FIGS. 6A and 6B show competition, by certain antibodies and human ACE2, for binding to RBD. Human ACE2 was located onto Octet pins, followed by association of RBD together with antibody or RBD alone. The vertical dashed line indicates the start of RBD or RBD plus antibody association. FIG. 6A shows results for four purified recombinant antibodies and for two antibodies used in the form of ExpiCHO culture supernatant (SN). FIG. 6B shows separate graphs for the four purified recombinant antibodies (left panel) and for the two antibodies used in the form of ExpiCHO culture supernatant (right panel).

FIG. 7 shows results from a neutralization of infection assay using certain antibodies against SARS-CoV-2 pseudotyped virus. Human monoclonal antibodies isolated from patients recovered from either COVID-19 or SARS infections were expressed recombinantly and were tested in neutralization assays against vesicular stomatitis virus (VSV) pseudotyped with SARS-CoV-2 Spike protein. Results are shown for four antibodies isolated from patients recovered from COVID-19 (S2N3, S2N6, S2X2, and S2X3) and one antibody isolated from a patient recovered from SARS (S309 (VH SEQ ID NO.:172; VL SEQ ID NO.:176; Pinto et al. Nature 583:290-295 (2020)). All antibodies were expressed as variants having M428L and N434S (“LS”) Fc mutations. Antibodies were tested at concentrations indicated on the x-axis. Symbols show means of duplicates.

FIG. 8 shows results from a neutralization of infection assay using a monoclonal antibody isolated from a patient who recovered from COVID-19 (S2X2), a monoclonal antibody isolated from a patient who recovered from SARS (S309 (VH SEQ ID NO.:172; VL SEQ ID NO.:176; Pinto et al. Nature 583:290-295 (2020)), and the combination of S2X2 and S309. Antibodies were expressed recombinantly (as LS Fc variants) and were tested in neutralization assays against murine leukemia virus (MLV) pseudotyped with SARS-CoV-2 Spike protein. The starting concentration for individual antibodies was 5 μg/ml. The starting concentration for the combination of antibodies was 10 μg/ml total antibody. The x-axis shows the total concentration of antibody. Symbols show means±SD of duplicates.

FIG. 9 shows binding of certain antibodies to the RBD of SARS-CoV-2. Culture supernatant of transfected ExpiCHO cells producing human monoclonal antibodies isolated from patients recovered from either COVID-19 (S2N3, S2N6, S2X2, S2X3) or SARS-CoV (S309 (VH SEQ ID NO.:172; VL SEQ ID NO.:176; Pinto et al. Nature 583:290-295 (2020)) infections were used. Antibody concentrations in the culture supernatant were determined by ELISA before the test. Protein A sensors (Bioforte) were hydrated before loading of antibody at 3 μg/ml in kinetics buffer for 1.5 minutes. RBD of SARS-CoV-2 (residues 331-550 of spike protein from BetaCoV/Wuhan-Hu-1/2019, accession number MN908947; produced in-house) at 5 μg/ml was associated for 5 minutes. RBD was allowed to dissociate for 10 minutes. The start of the dissociation phase is indicated by the vertical dashed line.

FIG. 10 shows competition, by human monoclonal antibody S2X2 and human ACE2, for binding to SARS-CoV-2 RBD and. Human ACE2-His (Sino Biological) was located onto anti-HIS (HIS1K) biosensors (Molecular Devices—ForteBio), followed by association of RBD together with antibody or RBD alone. RBD was pre-incubated with or without antibody at 15 μg/ml for 30 minutes before measurement of RBD association to ACE2-His for 10 minutes. Dissociation was recorded for 5 minutes. The vertical dashed line indicates the start of the dissociation phase. Antibody was in the form of cell culture supernatant from transfected ExpiCHO cells.

FIGS. 11A-11C show results from neutralization of infection assays using monoclonal antibodies isolated from patients who recovered from COVID-19 and a comparator monoclonal antibody isolated from a patient who recovered from SARS (S309 (VH SEQ ID NO.:172; VL SEQ ID NO.:176; Pinto et al. Nature 583:290-295 (2020)). FIG. 11A shows results for monoclonal antibodies S2D60, S2D22, S2D52, and S309. FIG. 11B shows results for monoclonal antibodies S2D32, S2D8, S2D38, and S309. FIG. 11C shows results for monoclonal antibodies S2D25, S2D19, S2D34, and S309. Antibodies were tested in neutralization assays against murine leukemia virus (MLV) pseudotyped with SARS-CoV-2 Spike protein. The x-axis shows the total concentration of antibody.

FIGS. 12A-12F show competition, of monoclonal antibodies isolated from patients who recovered from COVID-19 versus human ACE2, for binding to SARS-CoV-2 RBD. ELISA plates were coated with recombinant human ACE2. Coating was carried out with ACE2 at 2 ug/ml in PBS. Plates were incubated overnight at 4° C. and blocking was performed with blocker Casein (1% Casein from Thermofisher) for 1 hour at room temperature. Serial dilutions of monoclonal antibodies were incubated with SARS-CoV-2 RBD at 20 ng/ml (RBD fused with mouse Fc, from Sino Biological) for 30 minutes at 37° C. and then transferred onto the ACE2-coated plates for an additional incubation at room temperature. Plates were washed and binding of RBD to ACE2 was detected using a polyclonal goat anti-mouse Fc-AP antibody (Southern Biotech). After an additional wash, AP substrate pNPP (Sigma) was added and plates were incubated at 20 minutes at room temperature before measuring adsorbance at 405 nm with a spectrophotometer (Powerwave340 Biotek). FIG. 12A shows results for monoclonal antibodies S2D4, S2D5, S2D8, S2D10, and S2A4. FIG. 12B shows results for monoclonal antibodies S2D11, S2D15, S2D19, S2D22, and S2A4. FIG. 12C shows results for monoclonal antibodies S2D25, S2D27, S2D31, S2D32, and S2A4. FIG. 12D shows results for monoclonal antibodies S2D34, S2D38, S2D39, S2D41, and S2A4. FIG. 12E shows results for monoclonal antibodies S2D43, S2D47, S2D51, S2D52, and S2A4. FIG. 12F shows results for monoclonal antibodies S2D53, S2D60, and S2A4.

FIGS. 13A-13C show results from an RBD binding assay using monoclonal antibodies isolated from patients who recovered from COVID-19. FIG. 13A shows results for monoclonal antibodies S2D4, S2D5, S2D8, S2D10, S2D 11, S2D13, S2D15, S2D19, S2D22, S2D24, and S2D25. FIG. 13B shows results for monoclonal antibodies S2D27, S2D31, S2D32, S2D34, S2D38, S2D39, S2D41, S2D43, S2D47, S2D51, and S2D52. FIG. 13C shows results for monoclonal antibodies S2D53, S2D57, and S2D60. In these experiments, the antibodies were expressed as recombinant IgG1 with M428L and N434 (“LS”) Fc mutations. The antibodies shown in the figure key in bold font were cross-reactive with SARS-CoV RBD.

FIGS. 14A-14E show pair-wise competition of five monoclonal antibodies of the present disclosure (S2D8, S2D25, S2D32, S2D60, and S2D22) for binding to the RBD of SARS-CoV-2. Each of FIGS. 14A-14E shows results of the monoclonal antibody indicated at left in competition with each of the other antibodies (indicated along the top of the figure). FIG. 14A shows results for monoclonal antibody S2D8. FIG. 14B shows results for monoclonal antibody S2D25. FIG. 14C shows results for monoclonal antibody S2D32. FIG. 14D shows results for monoclonal antibody S2D60. FIG. 14E shows results for monoclonal antibody S2D22. The dashed vertical lines in each graph show the switch from the first antibody, indicated on the left of the figure, to the second antibody, indicated at the top of the graph.

FIG. 15 shows binding affinity and avidity of five monoclonal antibodies of the present disclosure to SARS-CoV-2 RBD, as measured by Octet. RBD was loaded to BLI pins and association of the indicated antibody was measured. Vertical dashed lines indicate the start of the dissociation phase when BLI pins were switched to buffer.

FIG. 16 shows data from a neutralization of infection assay using antibodies against authentic SARS-CoV-2 virus. The comparator antibody labeled “S309-v2” (also referred to herein as S309 N55Q) in FIG. 16 comprises the VH amino acid sequence set forth in SEQ ID NO.:340 (HCDRs of SEQ ID NOs.:341-343) and the VL amino acid sequence set forth in SEQ ID NO.:344 (LCDRs of SEQ ID NOs.:345-347). Vero E6 cells cultured in DMEM supplemented with 10% FBS (VWR) and 1×Penicillin/Streptomycin (Thermo Fisher Scientific) were seeded in white 96-well plates at 20,000 cells/well and attached overnight. Serial 1:4 dilutions of the monoclonal antibodies were incubated with 200 pfu of SARS-CoV-2 (isolate USA-WA1/2020, passage 3, passaged in Vero E6 cells) for 30 minutes at 37° C. in a BSL-3 facility. Cell supernatant was removed and the virus-antibody mixture was added to the cells. 24 hours post-infection, cells were fixed with 4% paraformaldehyde for 30 minutes, followed by two PBS (pH 7.4) washes and permeabilization with 0.25% Triton X-100 in PBS for 30 minutes. After blocking in 5% milk powder/PBS for 30 minutes, cells were incubated with a primary antibody targeting SARS-CoV-2 nucleocapsid protein (Sino Biological, cat. 40143-R001) at a 1:2000 dilution for 1 hour. After washing and incubation with a secondary Alexa647-labeled antibody mixed with 1 μg/ml Hoechst33342 for 1 hour, plates were imaged on an automated cell-imaging reader (Cytation 5, Biotek) and nucleocapsid-positive cells were counted using the manufacturer's supplied software. Data were processed using Prism software (GraphPad Prism 8.0).

FIGS. 17A and 17B show results from neutralization of infection assays using monoclonal antibodies. FIG. 17A shows results for monoclonal antibodies S2X127 and S2X129. FIG. 17B shows results for monoclonal antibodies S2X132 and S2X190. Antibodies were tested in neutralization assays against murine leukemia virus (MLV) pseudotyped with SARS-CoV-2 Spike protein. The x-axis shows the total concentration of antibody. Calculated IC50, IC80, and IC90 values are shown in the box on the right side of each figure.

FIGS. 18A and 18B show results from neutralization of infection assays using certain monoclonal antibodies. Human monoclonal antibodies were expressed recombinantly and were tested in neutralization assays against vesicular stomatitis virus (VSV) pseudotyped with SARS-CoV-2 Spike protein. FIG. 18A shows results for monoclonal antibodies S2X127, S2X129, and S2X132. FIG. 18B shows results for monoclonal antibody S2X190, and for comparator monoclonal antibodies S2X193 and S2X195. Antibodies were tested at concentrations indicated on the x-axis. Calculated IC50 and IC90 values are shown at the bottom of each figure.

FIG. 19 shows the ability of certain anti-SARS-CoV-2 monoclonal antibodies to inhibit binding by SARS-CoV-2 RBD to human ACE2. ELISA plates were coated with recombinant human ACE2 at 2 μg/ml in PBS. Serial dilutions of monoclonal antibodies were incubated with SARS-CoV-2 RBD at 20 ng/ml (RBD fused with mouse Fc, from Sino Biological) for 30 minutes at 37° C. and then transferred onto the ACE2-coated plates for an additional 20 minute incubation at room temperature. Eleven serial dilutions were used, starting at 10 μg/ml and diluting at 1:3. Binding of RBD to ACE2 was detected using secondary antibody goat F(ab′)2 anti-mouse IgG(H+L) antibody (Southern Biotech) conjugated to alkaline phosphatase, followed by addition of pNPP (Sigma Aldrich N2765-100TAB) in bicarbonate buffer and reading absorbance at 405 nm. Shown are results for monoclonal antibodies S2X127, S2X129, S2X132, and S2X190, along with comparator antibodies S2X193 and S2X195. Calculated IC50 values are shown to the right of the graph.

FIGS. 20A-20D show binding affinity and avidity of four monoclonal antibodies of the present disclosure to SARS-CoV-2 RBD, as measured by Octet. Antibody (as indicated in the bottom right of the figure) was loaded on Protein A pins at 2.7 μg/ml. SARS-CoV-2 RBD was loaded for 5 minutes at 6 μg/ml, 1.5 μg/ml, or 0.4 μg/ml. Dissociation was measured for 7 minutes. The vertical dashed line in each figure indicates the start of the dissociation phase.

FIG. 21 shows binding affinity and avidity of monoclonal antibodies S2X127, S2X129, S2X132, and S2X190, along with seven comparator antibodies, to SARS-CoV RBD, as measured by Octet. Antibody was loaded on Protein A pins at 2.7 μg/ml. SARS-CoV RBD was loaded for 5 minutes at 6 μg/ml. Dissociation was measured for 7 minutes. The vertical dashed line in each figure indicates the start of the dissociation phase. The top-to-bottom order of the curves in the graph corresponds to the top-to-bottom order of the antibody names to the right of the graph; i.e., antibody S2X127 corresponds to the top curve in the graph, and antibody S2X278 corresponds to the bottom curve in the graph.

FIGS. 22A-22C show results from neutralization of infection assays using certain monoclonal antibodies. FIG. 22A shows results for monoclonal antibody S2X227 (VH amino acid sequence set forth in SEQ ID NO.:398 (HCDRs set forth in SEQ ID NOs.:399-401); VL amino acid sequence set forth in SEQ ID NO.:402 (LCDRs set forth in SEQ ID NOs.:403-405). FIG. 22B shows results for monoclonal antibodies S2X200 and S2X259. S2X259 comprises the VH amino acid sequence set forth in SEQ ID NO.:408 (HCDRs of SEQ ID NOs.:409-411) and the VL amino acid sequence set forth in SEQ ID NO.:412 (LCDRs of SEQ ID NOs.:413-415). FIG. 22C shows results for monoclonal antibody S2X288. Antibodies were tested in neutralization assays against murine leukemia virus (MLLV) pseudotyped with SARS-CoV-2 Spike protein. The x-axis shows the total concentration of antibody. Calculated IC50, IC80, and IC90 values are shown in the box on the right side of each figure (left-hand column of the box; e.g., interpolated IC90 of S2X227 in FIG. 22A is 86.995 ng/mL).

FIGS. 23A and 23B show binding of human monoclonal antibodies S2X227 (also identified herein as “S2X227-v1”; VH amino acid sequence set forth in SEQ ID NO.:398 (HCDRs set forth in SEQ ID NOs.:399-401), VL amino acid sequence set forth in set forth in SEQ ID NO.:402 (LCDRs set forth in SEQ ID NOs.:403-405)) and S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) to SARS-CoV Spike protein, SARS-CoV Spike protein RBD, and SARS-CoV-2 Spike protein RBD. Human monoclonal antibodies were expressed recombinantly and binding was tested by ELISA. FIG. 23A shows binding of antibodies to SARS-CoV Spike protein RBD (top panel) and SARS-CoV Spike protein (bottom panel). FIG. 23B shows binding of antibodies to SARS-CoV-2 Spike protein RBD (top panel) and to an uncoated control plate (bottom panel). The boxes to the right of the graphs show calculated EC50 values.

FIGS. 24A and 24B show the ability of monoclonal antibodies to inhibit binding by SARS-CoV-2 RBD to human ACE2, as measured by ELISA. FIG. 24A shows results for monoclonal antibody S2X200, along with comparator antibody S2X179. FIG. 24B shows results for monoclonal antibodies S2X227 (VH amino acid sequence set forth in SEQ ID NO.:398 (HCDRs set forth in SEQ ID NOs.:399-401), VL amino acid sequence set forth in set forth in SEQ ID NO.:402 (LCDRs set forth in SEQ ID NOs.:403-405)) and S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)). Calculated IC50 values are shown in the box to the right of each graph.

FIGS. 25A and 25B show binding of human monoclonal antibody S2X200 and comparator antibody S2X179 to SARS-CoV Spike protein, SARS-CoV Spike protein RBD, and SARS-CoV-2 Spike protein RBD. Human monoclonal antibodies were expressed recombinantly and binding was tested by ELISA. FIG. 25A shows binding of antibodies to SARS-CoV Spike protein RBD (top panel) and SARS-CoV Spike protein (bottom panel). FIG. 25B shows binding of antibodies to SARS-CoV-2 Spike protein RBD (top panel) and to an uncoated control plate (bottom panel). The box to the right of the top graph in FIG. 25B shows calculated EC50 values for binding SARS-CoV-2 RBD.

FIG. 26 summarizes results of quantitative epitope-specific serology studies using monoclonal antibody S309 and other anti-Spike antibodies, as determined by binding competition, cryo-EM, and crystallography data. Underlined antibodies are cross-reactive with SARS-CoV.

FIGS. 27A-27C show neutralization of SARS-CoV-2 infection by certain monoclonal antibodies. FIG. 27A shows results for antibody S2X193, along with five comparator antibodies, including S309 N55Q LS. S309 N55Q LS comprises the VH sequence as set forth in SEQ ID NO.:340 (HCDRs of SEQ ID NOs.:341-343) and the VL sequence as set forth in SEQ ID NO.: 344 (LCDRs of SEQ ID NOs.:345-347), and comprises an MLNS (M428L/N434S; abbreviated in the figure as “LS”) mutation in the Fc region. FIG. 27B shows results for antibodies S2X195, S2X219, and S2X246, along with three comparator antibodies. FIG. 27C shows results for five antibodies along with comparator antibody S309 N55Q LS.

FIG. 28 shows binding to RBD by antibodies S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) and 407_10_1_v2 (an engineered variant of S2X259, having the VH amino acid sequence set forth in SEQ ID NO.:428 (HCDRs of SEQ ID NOs.:409-411) and the VL amino acid sequence set forth in SEQ ID NO.:412 (LCDRs of SEQ ID NOs.:413-415), as measured by ELISA.

FIG. 29 shows neutralization of SARS-CoV-2 infection by certain antibodies using a VSV pseudovirus. Data are from one single experiment, triplicate wells VSV-luc(spike D19) pseudovirus. “LS”=Fc mutations M428L+N434S. “S2X227-v1” comprises the VH amino acid sequence set forth in SEQ ID NO.:398 (HCDRs set forth in SEQ ID NOs.:399-401) and the VL amino acid sequence set forth in set forth in SEQ ID NO.:402 (LCDRs set forth in SEQ ID NOs.:403-405)). “S309 wt” comprises the VH amino acid sequence set forth in SEQ ID NO.: 172 and the VL amino acid sequence set forth in SEQ ID NO.:176.

FIG. 30 shows neutralization of infection by live SARS-CoV-2 by certain antibodies using a VSV pseudovirus. Data are from triplicate wells SARS-CoV-2-luc, MOI 0.1, 6h infection. “S2X227-v1” comprises the VH amino acid sequence set forth in SEQ ID NO.:398 (HCDRs set forth in SEQ ID NOs.:399-401) and the VL amino acid sequence set forth in set forth in SEQ ID NO. 402 (LCDRs set forth in SEQ ID NOs.:403-405)). “S309 wt” comprises the VH amino acid sequence set forth in SEQ ID NO.:172 and the VL amino acid sequence set forth in SEQ ID NO.:176.

FIGS. 31A and 31B show activation of FcγRIIIa (V158 allele) (FIG. 31A) and FcγRIIa (H131 allele) (FIG. 31B) by certain antibodies. Data show experiments using CHO target cells expressing SARS CoV2 S protein. “S2X227-v1” comprises the VH amino acid sequence set forth in SEQ ID NO.:398 (HCDRs set forth in SEQ ID NOs.:399-401) and the VL amino acid sequence set forth in set forth in SEQ ID NO.:402 (LCDRs set forth in SEQ ID NOs.:403-405)). “S309” comprises the VH amino acid sequence set forth in SEQ ID NO.:172 and the VL amino acid sequence set forth in SEQ ID NO.:176.

FIG. 32 shows a phylogenetic tree of sarbecovirus RBDs constructed via maximum likelihood analysis of amino acid sequences retrieved from GISAID and GenBank. Cross-reactivity within the sarbecovirus subgenus is shown for S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)), S2E12 (see Tortorici et al. Ultrapotent human antibodies protect against SARS-CoV-2 challenge via multiple mechanisms. Science 370, 950-957 (2020)), S309 (VH amino acid sequence set forth in SEQ ID NO.:172, VL amino acid sequence set forth in SEQ ID NO.:176; see Pinto et al. Cross-neutralization of SARS-CoV-2 by a human monoclonal SARS-CoV antibody. Nature 583, 290-295 (2020)), and ADG-2 (see Rappazzo et al. Broad and potent activity against SARS-like viruses by an engineered human monoclonal antibody. Science 371, 823-829 (2021)).

FIG. 33 shows flow cytometry analysis of S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) cross-reactivity with a panel of 30 S glycoproteins representative of sarbecovirus clades 1a, 1b, 2, and 3 as well as SARS-CoV-2 variants of concern (VOCs). One independent experiment out of at least two is shown.

FIG. 34 shows S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) binding to RBDs representative of the different sarbecovirus clades and SARS-CoV-2 variants as measured by ELISA. One independent experiment out of at least two is shown. Error bars indicate standard deviation of duplicates or triplicates.

FIG. 35 shows S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415))-mediated neutralization of SARS-CoV-2-Nluc authentic virus and SARS-CoV-2 S MLV-pseudotyped virus. One independent experiment out of at least two is shown. Error bars indicate standard deviation of duplicates or triplicates.

FIG. 36 shows S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415))-mediated neutralization of VSV pseudotypes harbouring SARS-CoV-2 S from isolates representing the B.1.1.7, B.1.351, P.1 and B.1.429 VOC (top panel) as well as single RBD mutants (bottom panel). One independent experiment out of at least two is shown. Error bars indicate standard deviation of duplicates or triplicates.

FIG. 37 shows S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415))-mediated neutralization of VSV pseudotypes harboring SARS-CoV-related (clade 1a, top panel) or SARS-CoV-2-related (clade 1b, bottom panel) S glycoproteins. One independent experiment out of at least two is shown. Error bars indicate standard deviation of duplicates or triplicates.

FIGS. 38A-38C show S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) Fab binding to recombinant sarbecovirus RBDs, prefusion SARS-CoV-2 S ectodomain trimer and RBD variants analysed by surface plasmon resonance. S or RBD antigen was captured on the sensor chip surface and binding to S2X259 Fab at 11, 33, 100, and 300 nM was measured successively, in single-cycle kinetics format. All data have been fit to a 1:1 binding model and the equilibrium dissociation constant (KD) is reported. For the S-binding data, an apparent KD (KD,app) is reported since the kinetics incorporate conformational dynamics between open and closed RBD states.

FIG. 39 shows frequency of mutations identified as not affecting, affecting, or having as-yet unknown effect on S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) binding or neutralization in circulating SARS-CoV-2 isolates as of March 2021; as of April 2021, the G504D mutation which reduces neutralization by S2X259 has been found in 0.001% of 232,598 viral isolates having a naturally occurring mutation in S2X259 epitope; the 232,598 viral isolates account for approximately 27.4% of all sequences currently available.

FIG. 40 shows S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) in vitro neutralizing activity vs. VSV-based SARS-COV-2 S mutations. For each mutant, the fold change vs neutralization against SARS-CoV-2 S WT (Wuhan-Hu-1) is reported. *Q506K mutant displayed a 10-fold reduction in viral entry in comparison the other mutants.

FIG. 41 shows protein sequence alignment of representative sarbecovirus RBDs, with matching residues shown as dots and conservation indicated as a bar plot. Positions are based on SARS-CoV-2 RBD. Residues determined to be important for S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) binding, as well as extended epitope, are denoted. Substitutions at positions D405 and G504 are indicated in the alignment. The sarbecovirus RBDs shown include representatives of clade 1a (SARS-CoV, WIV1, RsSHC014, LYRa3, CS24, A021, Rs3367, HKU3, PC4-127, Rs4231, and Rs4084), clade 1b (SARS-CoV-2, RaTG13, PG-GD-2019, and PG-GX-2017), clade 2 (SX2011, YN2013, Anlong112, Rs4255, YN2011, SC2018, ZC45, ZXC21, RmYN02, Rml/2004, Rf1-2004, Rf4092, and As6526) and clade 3 (BtkY72 and BGR/2008).

FIG. 42 shows inhibition of RBD binding to ACE2. Pre-incubation of serial dilutions of S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) with SARS-CoV-2 RBD (grey) or SARS-CoV RBD (black) prevents binding to immobilized human ACE2 (hACE2) ectodomain in ELISA.

FIG. 43 shows mAb-mediated S1 subunit shedding from cell-surface expressed SARS-CoV-2 S, as determined by flow-cytometry. S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) was examined; S2E12 was included as positive control; S2M11 was included as negative control. CHO cells stably expressing wild-type SARS-CoV-2 S were resuspended in wash buffer (PBS 1% BSA, 2 mM EDTA) and treated with 10 μg/mL TPCK-trypsin (Worthington Biochem) for 30 min at 37° C. Cells were then washed and distributed into round bottom 96-well plates (90,000 cells/well). S2X259 was added to cells at 15 g/mL final concentration for 180 min at 37° C. Cells were collected at different time points (5, 30, 60, 120 and 180), washed with wash buffer at 4° C., and incubated with 1.5 g/mL secondary goat anti-human IgG, Fc fragment specific (Jackson ImmunoResearch) on ice for 20 min. Cells were washed and resuspended in wash buffer and analyzed with ZE5 FACS (Bio-rad).

FIG. 44 shows quantification of viral RNA loads (left panel) and replicating virus titres (TCID50) (right panel) in the lungs of Syrian hamsters 4 days post intranasal challenge with B.1.351 SARS-CoV-2 VOC following prophylactic administration of S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) at 1 mg/kg (n=6), 4 mg/kg (n=6), and in combination with S309 (1+1 mg/kg, n=6). Mann-Whitney test was used for statistical analysis of significance. *p<0.05, **p<0.01. Data from one independent experiment are presented. Briefly, SARS-CoV-2 Wuhan (BetaCov/Belgium/GHB-03021/2020-EPI ISL 109 407976|2020-02-03) and B.1.351 (hCoV105 19/Belgium/rega-1920/2021; EPI_ISL_896474, 2021-01-11) isolates were recovered from nasopharyngeal swabs taken from a RT-qPCR confirmed asymptomatic patient and from a patient with respiratory symptoms, respectively. A close relatedness with the prototypic Wuhan-Hu-1 2019 SARS-CoV-2 and with B.1.351 lineage was confirmed by sequencing and phylogenetic analysis. Infectious viruses were isolated by serial passaging on Vero E6 cells and passage 6 for SARS-CoV-2 Wuhan and passage 2 for B.1.351 viruses were used for the study. The titre of the virus stock was determined by end-point dilution on Vero E6 cells by the Reed and Muench method. Syrian hamsters (Mesocricetus auratus) were purchased from Janvier Laboratories and were housed per two in ventilated isolator cages (IsoCage N Biocontainment System, Tecniplast) with ad libitum access to food and water and cage enrichment (wood block). Housing conditions and experimental procedures were approved by the ethical committee of animal experimentation of KU Leuven (license P065-2020). 6-10 week-old female hamsters were administered by intraperitoneal injection with S2X259 mAb at 1 mg/kg and 4 mg/kg 48 hours before intranasal infection with 1.89×106 TCID50 in 50 μl inoculum. Hamsters were monitored for appearance, behavior and weight. At day 4 post infection hamsters were euthanized by intraperitoneal injection of 500 μL Dolethal (200 mg/mL sodium pentobarbital, Vetoquinol SA). Lungs were collected, homogenized using bead disruption (Precellys) in 350 μL RLT buffer (RNeasy Mini kit, Qiagen) and centrifuged (10,000 rpm, 5 minutes, 4° C.) to pellet the cell debris. RNA was extracted using a NucleoSpin kit (Macherey-Nagel) according to the manufacturer's instructions. RT-qPCR was performed on a LightCycler96 platform (Roche) using the iTaq Universal Probes One-Step RTqPCR kit (BioRad) with N2 primers and probes targeting the nucleocapsid. Standards of SARS-CoV-2 cDNA (IDT) were used to express viral genome copies per mg tissue or per mL serum. To quantify infectious SARS-CoV-2 particles, endpoint titrations were performed on confluent Vero E6 cells in 96-well plates. Viral titres were calculated by the Reed and Muench method and were expressed as 50% tissue culture infectious dose (TCID50) per mg tissue.

FIG. 45 shows viral RNA loads (left panel) and replicating virus titers (right panel) in the lungs of Syrian hamsters 4 days post-intranasal infection with prototypic SARS-CoV-2. Results for one independent experiment are shown. Irrelevant mAb n=3; S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) 4 mg/kg n=4.

FIG. 46 shows data from competition binding assays for S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) vs site I-targeting S2E12 (top panel) and site IV-targeting S309 (VH amino acid sequence set forth in SEQ ID NO.:172, VL amino acid sequence set forth in SEQ ID NO.:176) (bottom panel) mAbs on SARS-CoV-2 RBD as measured by biolayer interferometry. One independent experiment out of two is shown.

FIG. 47 shows correlation between concentration of monoclonal antibodies measured in the serum before infection (day 0; x-axis) and infectious virus (TCID50) in the lung four days post infection (y-axis). Syrian hamsters were intra-nasally challenged with B.1.351 SARS-CoV-2 following prophylactic administration of antibody S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) or a combination of antibodies S2X259 and S309 (VH amino acid sequence set forth in SEQ ID NO.:172, VL amino acid sequence set forth in SEQ ID NO.:176) (see also FIG. 44). Indicated by circles are: S2X259, 1 mg/kg; S2X259, 4 mg/kg; and S309+S2X259, 1+1 mg/kg. Data from one independent experiment are presented.

FIG. 48 shows quantification of viral RNA load (RNA genome copies/mg lung) in the lungs of Syrian hamsters four days post intra-nasal infection with prototypic (Wuhan-1 related) SARS-CoV-2 following prophylactic administration of S2X259 antibody (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)), plotted as a function of serum monoclonal antibody concentrations before infection (day 0). S2X259 antibody was administered at 4 mg/kg; n=4.

FIG. 49 shows quantification of replicating virus titers (TCID50) in the lungs of Syrian hamsters four days post intra-nasal infection with prototypic (Wuhan-1 related) SARS-CoV-2 following prophylactic administration of S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) antibody, plotted as a function of serum monoclonal antibody concentrations before infection (day 0). S2X259 antibody was administered at 4 mg/kg; n=4.

FIG. 50 shows S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) in vitro neutralizing activity vs. VSV pseudoviruses harboring S mutations to S2X259-contact residues found with higher frequency in 229 clinical isolates. For each mutant the fold change vs neutralization against SARS-CoV-2 S WT is reported. *Q506K mutant displayed a 10-fold reduction in viral entry in comparison the other mutants. Results from two independent experiments are reported.

FIG. 51 shows infection of HEK293T cells transfected to overexpress ACE2 or one of a panel of selected lectins and receptor candidates by VSV-SARS-CoV-2 pseudovirus.

FIG. 52 shows micrographs of stable HEK293T cell lines overexpressing DC-SIGN, L-SIGN, SIGLEC1, or ACE2 infected with authentic SARS-CoV-2 (MOI of 0.1), then fixed and immunostained for 24 hours for SARS-CoV-2 nucleoprotein.

FIG. 53 shows quantification of luciferase levels in stable HEK293T cell lines overexpressing DC-SIGN, L-SIGN, SIGLEC1, or ACE2, as measured 24 hours after infection with SARS-CoV-2-Nluc.

FIG. 54 shows quantification of luciferase levels in stable HEK293T cell lines overexpressing DC-SIGN, L-SIGN, SIGLEC1, or ACE2 after incubation with different concentrations of anti-SIGLEC1 monoclonal antibody (clone 7-239) and infection with SARS-CoV-2-Nluc.

FIG. 55 shows infection of cells transiently transduced to overexpress DC-SIGN, L-SIGN, SIGLEC1, or ACE2 by VSV-SARS-CoV-2 pseudovirus. Results for HEK293T cells (left panel), HeLa cells (center panel), and MRC5 cells (right panel) are shown.

FIG. 56 shows infection of stable HEK293T cell lines overexpressing DC-SIGN, L-SIGN, SIGLEC1, or ACE2 after treatment with ACE2 siRNA followed by infection with VSV-SARS-CoV-2 pseudovirus.

FIG. 57 shows infection of stable HEK293T cell lines overexpressing DC-SIGN, L-SIGN, SIGLEC1, or ACE2 after treatment with different concentrations of anti-ACE2 antibody (polyclonal serum) followed by infection with VSV-SARS-CoV-2 pseudovirus.

FIG. 58 shows distribution and expression of ACE2, DC-SIGN (CD209), L-SIGN (CLEC4M), and SIGLEC1 in the human lung cell atlas.

FIG. 59 shows analysis of major cell types with detectable SARS-CoV-2 genome in bronchoalveolar lavage fluid or sputum of severe COVID-19 patients. The single cell gene expression profiles are shown as a t-SNE (t-distributed stochastic neighbor embedding) plot, identified by cell type and sized by viral load.

FIG. 60 shows analysis of major cell types with detectable SARS-CoV-2 genome in bronchoalveolar lavage fluid or sputum of severe COVID-19 patients. The cumulative fraction of cells (y-axis) with detected viral RNA per cell up to the corresponding log CPM (log(counts per million); x-axis) is shown for each of the indicated cell types.

FIG. 61 shows a heatmap matrix of counts for cells with detected transcripts for the receptor genes shown on the x-axis and SARS-CoV-2+ cell types on the y-axis. Total n=3,085 cells from eight subjects. See Ren, X. et al. COVID-19 immune features revealed by a large-scale single cell transcriptome atlas. Cell, doi:10.1016/j.cell.2021.01.053 (2021).

FIG. 62 shows correlation of receptor transcript counts (y-axis of each plot) with SARS-CoV-2 RNA counts (x-axis of each plot) in macrophages and in secretory cells. Correlation is based on counts before log transformation from Ren et al.

FIG. 63 shows results of trans-infection with VSV-SARS-CoV-2. A schematic of the trans-infection process is shown in the left panel. HeLa cells transduced with DC-SIGN, L-SIGN, or SIGLEC1 were incubated with VSV-SARS-CoV-2, extensively washed, and co-cultured with Vero-E6-TMPRSS2 susceptible target cells. Results in the presence or absence of target cells are shown in the right panel.

FIG. 64 shows results of trans-infection, where VSV-SARS-CoV-2 viral adsorption was performed in the presence or absence of an anti-SIGLEC1 blocking antibody.

FIG. 65 shows quantification of binding of purified, fluorescently-labeled SARS-CoV-2 spike protein or RBD to the indicated cell lines, as measured by flow cytometry. “A” indicates cell line overexpressing ACE2; “T” indicates cell line overexpressing TMPRSS2.

FIG. 66 shows quantification of cellular ACE2 and TMPRSS2 transcripts in the indicated cell lines, as measured by RT-qPCR. “A” indicates cell line overexpressing ACE2; “T” indicates cell line overexpressing TMPRSS2.

FIG. 67 shows CHO-S cell-cell fusion mediated by different spike-specific antibodies. Fusion was quantified using the Cytation 5 Imager (BioTek) and an object detection protocol that detected nuclei as objects and measured their size. The area of the objects in fused cells divided by the total area of all the objects multiplied by 100 provides the percentage of fused cells.

FIG. 68 shows inhibition of S2E12-induced cell-cell fusion of CHO-S cells by 15 μg/ml of the indicated antibodies.

FIG. 69 shows S2E12-induced uni-directional fusion (also referred to as trans-fusion) of S-positive CHO-S cells with fluorescently-labelled S-negative CHO cells in the absence of ACE2. Nuclei were stained with Hoechst dye; cytoplasm was stained with CellTracker Green.

FIG. 70 shows analysis of binding of antibodies targeting DC/L-SIGN, DC-SIGN, SIGLEC1, or ACE2 on HEK293T cells stably over-expressing the respective attachment receptor, as measured by flow cytometry.

FIG. 71 shows analysis of binding of antibodies targeting DC/L-SIGN, DC-SIGN, SIGLEC1, or ACE2 on HEK293T cells stably over-expressing the respective attachment receptor, as measured by immunofluorescence.

FIG. 72 shows infection of HEK293T cells stably over-expressing the indicated attachment receptor by VSV-SARS-CoV-2 pseudotyped with wild type spike protein (dark grey bars), or VSV-SARS-CoV-2 pseudotyped with spike protein bearing the mutations of the B1.1.7 lineage (light grey bars). Luminescence was analyzed one day post infection.

FIG. 73 shows quantification of binding of purified, fluorescently-labelled SARS-CoV-2 spike protein (left panels) or RBD (right panels) to the indicated cell lines, as measured by flow cytometry.

FIG. 74 shows quantification of binding of purified, fluorescently-labelled SARS-CoV-2 spike protein (left panels) or RBD (right panels) to the indicated cell lines, as measured by flow cytometry.

FIG. 75 shows binding of immunocomplexes to hamster splenocytes. Alexa-488 fluorescent immunocomplexes (IC) were titrated (0-200 nM range) and incubated with total naïve hamster splenocytes. Binding was revealed with a cytometer upon exclusion of dead/apoptotic cells and physical gating on bona fide monocyte population. Left panel shows the fluorescent intensity associated to hamster cells of IC made with either hamster or human Fc antibodies (Human S309 shown in green; GH-S309 shown in dark grey; GH-S309-N297A shown in blue). A single replicate of two is shown. Right panel shows the relative Alexa-488 mean fluorescent intensity of the replicates measured on the entire monocyte population.

FIG. 76 shows analysis of the role of host effector function in SARS-CoV-2 challenge. Syrian hamsters were injected with the indicated amount (mg/kg) of hamster IgG2a S309, either wt or Fc silenced (S309-N297A). Top panel shows quantification of viral RNA in the lung 4 days post infection. Center panel shows quantification of replicating virus in the lung 4 days post infection. Bottom panel shows histopathological score in the lung 4 days post infection. Control animals (white symbols) were injected with 4 mg/kg unrelated control isotype antibody. * p<0.05, ** p<0.01, *** p<0.001, **** p<0.0001 vs control animals, using Mann-Whitney test.

### DETAILED DESCRIPTION

Provided herein are antibodies and antigen-binding fragments that are capable of binding to SARS-CoV-2 coronavirus (e.g., a SARS-CoV-2 surface glycoprotein and/or RBD, as described herein, in a SARS-CoV-2 virion and/or expressed on the surface of a host cell, such as a cell infected by the SARS-CoV-2 coronavirus). A host cell can be, for example, a lung cell, a CHO cell (such as, for example, an ExpiCHO cell transfected to express the surface glycoprotein), or the like. In certain embodiments, presently disclosed antibodies and antigen-binding fragments can neutralize a SARS-CoV-2 infection in an in vitro model of infection and/or in a human subject.

In certain embodiments, presently disclosed antibodies and antigen-binding fragments are capable of binding to and/or neutralizing two, three, or more sarbecoviruses and/or SARS-CoV-2 viruses, such as, for example, a sarbecovirus of clade 1a, a sarbecovirus of clade 1b, a sarbecovirus of clade 2, a sarbecovirus of clade 3, and/or a variant of SARS-CoV-2.

Also provided are polynucleotides that encode the antibodies and antigen-binding fragments, vectors, host cells, and related compositions, as well as methods of using the antibodies, nucleic acids, vectors, host cells, and related compositions to treat (e.g., reduce, delay, eliminate, or prevent) a SARS-CoV-2 infection in a subject and/or in the manufacture of a medicament for treating a SARS-CoV-2 infection in a subject.

Further provided herein are antibodies and antigen-binding fragments that are capable of binding to multiple sarbecoviruses (e.g., a surface glycoprotein, as described herein, of one or more (e.g., one, two, three, four, five, six, or more) different sarbecovirus virions and/or expressed on the surface of a cell infected by two or more sarbecoviruses). In certain embodiments, presently disclosed antibodies and antigen-binding fragments can neutralize infection by two or more sarbecoviruses in an in vitro model of infection and/or in a human subject. Also provided are polynucleotides that encode the antibodies and antigen-binding fragments, vectors, host cells, and related compositions, as well as methods of using the antibodies, nucleic acids, vectors, host cells, and related compositions to treat (e.g., reduce, delay, eliminate, or prevent) infection by two or more sarbecoviruses in a subject and/or in the manufacture of a medicament for treating infection in a subject by two or more sarbecoviruses.

Prior to setting forth this disclosure in more detail, it may be helpful to an understanding thereof to provide definitions of certain terms to be used herein. Additional definitions are set forth throughout this disclosure.

As used herein, “sarbecovirus” refers to any betacoronavirus within lineage B, and includes lineage B viruses in clade 1a, clade 1b, clade 2, and clade 3. Examples of clade 1a sarbecoviruses are SARS-CoV and Bat SARS-like coronavirus WIV1 (WIV1). Examples of clade 1b sarbecoviruses are SARS-CoV-2, RatG13, Pangolin-Guanxi-2017 (PANG/GX) and Pangolin-Guangdon-2019 (PANG/GD). Examples of clade 2 sarbecoviruses are Bat ZC45 (ZC45), Bat ZXC21 (ZXC21), YN2013, and RmYN02. Examples of clade 3 sarbecoviruses are BtkY72 and BGR2008.

In some embodiments, an antibody or antigen-binding fragment thereof is capable of binding to: a sarbecovirus of clade 1a (e.g., SARS-CoV, WIV1, or both); a sarbecovirus of clade 1b (e.g., SARS-CoV-2, RatG13, Pangolin-Guanxi-2017 (PANG/GX), Pangolin-Guangdon-209, or any combination thereof); a sarbecovirus of clade 2; or a sarbecovirus of clade 3.

In certain further embodiments, an antibody or antigen-binding fragment thereof is capable of binding to a SARS-CoV-2 variant; e.g., a N501Y variant; a Y453F variant; a N439K variant; a K417V variant; a N501Y-K417N-E484K variant; a E484K variant; a California variant; a Brazilian variant; a Swiss variant; or any combination thereof.

In some embodiments, an antibody or antigen-binding fragment thereof is capable of inhibing a binding interaction between human ACE2 and a sarbecovirus (e.g., SARS-CoV-2) receptor binding domain (RBD) with an IC50 of about 12 ng/mL, about 12.5 ng/mL, or about 13 ng/mL.

As used herein, “SARS-CoV-2”, also referred to herein as “Wuhan seafood market phenomia virus”, or “Wuhan coronavirus” or “Wuhan CoV”, or “novel CoV”, or “nCoV”, or “2019 nCoV”, or “Wuhan nCoV” is a betacoronavirus believed to be of lineage B (sarbecovirus). SARS-CoV-2 was first identified in Wuhan, Hubei province, China, in late 2019 and spread within China and to other parts of the world by early 2020. Symptoms of SARS-CoV-2 infection include fever, dry cough, and dyspnea.

The genomic sequence of SARS-CoV-2 isolate Wuhan-Hu-1 is provided in SEQ ID NO.:1 (see also GenBank MN908947.3, Jan. 23, 2020), and the amino acid translation of the genome is provided in SEQ ID NO.:2 (see also GenBank QHD43416.1, Jan. 23, 2020). Like other coronaviruses (e.g., SARS-CoV-1), SARS-CoV-2 comprises a “spike” or surface (“S”) type I transmembrane glycoprotein containing a receptor binding domain (RBD). RBD is believed to mediate entry of the lineage B SARS coronavirus to respiratory epithelial cells by binding to the cell surface receptor angiotensin-converting enzyme 2 (ACE2). In particular, a receptor binding motif (RBM) in the virus RBD is believed to interact with ACE2.

The amino acid sequence of the SARS-CoV-2 Wuhan-Hu-1 surface glycoprotein is provided in SEQ ID NO.:3. Antibodies and antigen-binding fragments of the present disclosure are capable of binding to a SARS CoV-2 surface glycoprotein (S), such as that of Wuhan-Hu-1. For example, in certain embodiments, an antibody or antigen-binding fragment binds to an epitope in Wuhan-Hu-1 S protein RBD.

The amino acid sequence of SARS-CoV-2 Wuhan-Hu-1 RBD is provided in SEQ ID NO.:4. SARS-CoV-2 S protein has approximately 73% amino acid sequence identity with SARS-CoV S protein. The amino acid sequence of SARS-CoV-2 RBM is provided in SEQ ID NO.:5. SARS-CoV-2 RBD has approximately 75% to 77% amino acid sequence similarity to SARS-CoV-1 RBD, and SARS-CoV-2 RBM has approximately 50% amino acid sequence similarity to SARS-CoV RBM.

Unless otherwise indicated herein, SARS-CoV-2 Wuhan-Hu-1 refers to a virus comprising the amino acid sequence set forth in any one or more of SEQ ID NOs.:2, 3, and 4, optionally with the genomic sequence set forth in SEQ ID NO.: 1.

There have been a number of emerging SARS-CoV-2 variants. Some SARS-CoV-2 variants contain an N439K mutation, which has enhanced binding affinity to the human ACE2 receptor (Thomson, E. C., et al., The circulating SARS-CoV-2 spike variant N439K maintains fitness while evading antibody-mediated immunity. bioRxiv, 2020). Some SARS-CoV-2 variants contain an N501Y mutation, which is associated with increased transmissibility, including the lineages B.1.1.7 (also known as 20I/501Y.V1 and VOC 202012/01; (del69-70, del144, N501Y, A570D, D614G, P681H, T716I, S982A, and D1118H mutations)) and B.1.351 (also known as 20H/501Y.V2; L18F, D80A, D215G, R246I, K417N, E484K, N501Y, D614G, and A701V mutations), which were discovered in the United Kingdom and South Africa, respectively (Tegally, H., et al., Emergence and rapid spread of a new severe acute respiratory syndrome-related coronavirus 2 (SARS-CoV-2) lineage with multiple spike mutations in South Africa. medRxiv, 2020: p. 2020.12.21.20248640; Leung, K., et al., Early empirical assessment of the N501Y mutant strains of SARS-CoV-2 in the United Kingdom, October to November 2020. medRxiv, 2020: p. 2020.12.20.20248581). B.1.351 also include two other mutations in the RBD domain of SARS-CoV2 spike protein, K417N and E484K (Tegally, H., et al., Emergence and rapid spread of a new severe acute respiratory syndrome-related coronavirus 2 (SARS-CoV-2) lineage with multiple spike mutations in South Africa. medRxiv, 2020: p. 2020.12.21.20248640). Other SARS-CoV-2 variants include the Lineage B.1.1.28, which was first reported in Brazil; the Variant P.1, lineage B.1.1.28 (also known as 20J/501Y.V3), which was first reported in Japan; Variant L452R, which was first reported in California in the United States (Pan American Health Organization, Epidemiological update: Occurrence of variants of SARS-CoV-2 in the Americas, Jan. 20, 2021, available at reliefweb.int/sites/reliefweb.int/files/resources/2021-jan-20-phe-epi-update-SARS-CoV-2.pdf). Other SARS-CoV-2 variants include a SARS CoV-2 of clade 19A; SARS CoV-2 of clade 19B; a SARS CoV-2 of clade 20A; a SARS CoV-2 of clade 20B; a SARS CoV-2 of clade 20C; a SARS CoV-2 of clade 20D; a SARS CoV-2 of clade 20E (EU1); a SARS CoV-2 of clade 20F; a SARS CoV-2 of clade 20G; and SARS CoV-2 B1.1.207; and other SARS CoV-2 lineages described in Rambaut, A., et al., A dynamic nomenclature proposal for SARS-CoV-2 lineages to assist genomic epidemiology. Nat Microbiol 5, 1403-1407 (2020). The foregoing SARS-CoV-2 variants, and the amino acid and nucleotide sequences thereof, are incorporated herein by reference.

SARS-CoV is another betacoronavirus of lineage B (sarbecovirus) that causes respiratory symptoms in infected individuals. The genomic sequence of SARS-CoV Urbani strain has GenBank accession number AAP13441.1. The amino acid sequence of the SARS-CoV surface glycoprotein (“S protein”) is provided in SEQ ID NO: 450.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means+20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

“Nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, viral genomic RNA, and synthetic RNA, and polydeoxyribonucleic acid (DNA), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (K_{D}) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity).

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mites et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” or “vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vectors” include HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based antibody or antigen-binding fragment-encoding plasmid vectors for direct administration to a subject, are described further herein.

As used herein, the term “host” refers to a cell or microorganism targeted for genetic modification with a heterologous nucleic acid molecule to produce a polypeptide of interest (e.g., an antibody of the present disclosure).

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In the context of a SARS-CoV-2 infection (or infection by another sarbecovirus), a “host” refers to a cell or a subject infected with the SARS-CoV-2 coronavirus (or other sarbecovirus).

“Antigen” or “Ag”, as used herein, refers to an immunogenic molecule that provokes an immune response. This immune response may involve antibody production, activation of specific immunologically-competent cells, activation of complement, antibody dependent cytotoxicicity, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen. Antigens can also be present in a sarbecovirus, e.g. SARS-CoV-2 coronavirus (e.g., a surface glycoprotein or portion thereof), such as present in a virion, or expressed or presented on the surface of a cell infected by SARS-CoV-2.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

### Antibodies, Antigen-Binding Fragments, and Compositions

In one aspect, the present disclosure provides an isolated antibody, or an antigen-binding fragment thereof, that comprises a heavy chain variable domain (VH) comprising a CDRH1, a CDRH2, and a CDRH3, and a light chain variable domain (VL) comprising a CDRL1, a CDRL2, and a CDRL3, and is capable of binding to a surface glycoprotein of SARS-CoV-2. In certain embodiments, the antibody or antigen-binding fragment is capable of binding to a surface glycoprotein of SARS-CoV-2, such as expressed on a cell surface of a host cell and/or on a SARS-CoV-2 virion.

In certain embodiments, an antibody or antigen-binding fragment of the present disclosure associates with or unites with a SARS-CoV-2 surface glycoprotein epitope or antigen comprising the epitope, while not significantly associating or uniting with any other molecules or components in a sample.

In certain embodiments, an antibody or antigen-binding fragment of the present disclosure associates with or unites (e.g., binds) to a SARS-CoV-2 surface glycoprotein epitope, and can also associate with or unite with an epitope from another coronavirus (e.g., SARS-CoV) present in the sample, but not significantly associating or uniting with any other molecules or components in the sample. In other words, in certain embodiments, an antibody or antigen binding fragment of the present disclosure is cross-reactive for SARS-CoV-2 and one or more additional coronavirus.

In certain embodiments, an antibody or antigen-binding fragment of the present disclosure is capable of binding to a surface glycoprotein of two or more sarbecoviruses. In some embodiments, the two or more sarbecoviruses are selected from: clade 1a sarbecoviruses and/or clade 1b sarbecoviruses; clade 2 sarbecoviruses; clade 3 sarbecoviruses; or naturally occurring variants thereof, and any combination thereof. In certain embodiments, the antibody or antigen-binding fragment is capable of binding to a surface glycoprotein of two or more sarbecoviruses; e.g., capable of binding when a sarbecovirus is expressed on a cell surface of a host cell and/or on a sarbecovirus virion. In certain embodiments, the two or more sarbecoviruses are selected from SARS-CoV, WIV1, SARS-CoV2, PANG/GD, PANG/GX, RatG13, ZXC21, ZC45, RmYN02, BGR2008, BtkY72, and naturally occurring variants thereof. In some embodiments, the two or more sarbecoviruses include one or more of SARS-CoV-2 variants P.1, B.1.1.7, B.1.429, and B.1.351. In some embodiments, the two or more sarbecoviruses include one or more SARS-CoV-2 variants having S protein mutations N501Y, Y453F, N439K, K417V, E484K, or any combination thereof.

In certain embodiments, an antibody or antigen-binding fragment of the present disclosure associates with or unites with a sarbecovirus surface glycoprotein epitope or antigen comprising the epitope, while not significantly associating or uniting with any other molecules or components in a sample. In some embodiments, the epitope is comprised in a S1 subunit of a spike (S) protein. In further embodiments, the epitope is comprised in a receptor binding domain (RBD) of a S protein. In some embodiments, the epitope is a conformational epitope or a linear epitope.

In certain embodiments, an antibody or antigen-binding fragment of the present disclosure associates with or unites (e.g., binds) to a first sarbecovirus surface glycoprotein epitope, and can also associate with or unite with an epitope from another sarbecovirus present in the sample, but not significantly associating or uniting with any other molecules or components in the sample. In other words, in certain embodiments, an antibody or antigen binding fragment of the present disclosure is cross-reactive against and specifically binds to two or more sarbecoviruses.

In certain embodiments, an antibody or antigen-binding fragment of the present disclosure specifically binds to a sarbecovirus surface glycoprotein, such as a SARS-CoV-2 surface glycoprotein. As used herein, “specifically binds” refers to an association or union of an antibody or antigen-binding fragment to an antigen with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Alternatively, affinity may be defined as an equilibrium dissociation constant (K_{D}) of a particular binding interaction with units of M (e.g., 10⁻⁵ M to 10⁻¹³ M). Antibodies may be classified as “high-affinity” antibodies or as “low-affinity” antibodies. “High-affinity” antibodies refer to those antibodies having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low-affinity” antibodies refer to those antibodies having a Kₐ of up to 107 M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M).

In some contexts, antibody and antigen-binding fragments may be described with reference to affinity and/or to avidity for antigen. Unless otherwise indicated, avidity refers to the total binding strength of an antibody or antigen-binding fragment thereof to antigen, and reflects binding affinity, valency of the antibody or antigen-binding fragment (e.g., whether the antibody or antigen-binding fragment comprises one, two, three, four, five, six, seven, eight, nine, ten, or more binding sites), and, for example, whether another agent is present that can affect the binding (e.g., a non-competitive inhibitor of the antibody or antigen-binding fragment).

A variety of assays are known for identifying antibodies of the present disclosure that bind a particular target, as well as determining binding domain or binding protein affinities, such as Western blot, ELISA (e.g., direct, indirect, or sandwich), analytical ultracentrifugation, spectroscopy, and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known.

In certain examples, binding can be determined by recombinantly expressing a sarbecovirus antigen, such as a SARS-CoV-2 antigen in a host cell (e.g., by transfection) and immunostaining the (e.g., fixed, or fixed and permeabilized) host cell with antibody and analyzing binding by flow cytometery (e.g., using a ZE5 Cell Analyzer (BioRad®) and FlowJo software (TreeStar). In some embodiments, positive binding can be defined by differential staining by antibody of SARS-CoV-2-expressing cells versus control (e.g., mock) cells.

In some embodiments, an antibody or antigen-binding fragment of the present disclosure binds to a sarbecovirus spike protein (i.e., from two or more sarbecoviruses) expressed on the surface of a host cell (e.g., an Expi-CHO cell), as determined by flow cytometry.

In some embodiments an antibody or antigen-binding fragment of the present disclosure binds to a sarbecorvirus S protein, such as a SARS-CoV-2 S protein, as measured using biolayer interferometry. In certain embodiments, an antibody or antigen-binding fragment of the present disclosure binds to SARS-CoV-2 S protein with a K_{D} of less than about 4.5×10⁻⁹ M, less than about 5×10⁻⁹ M, less than about 1×10⁻¹⁰ M, less than about 5×10⁻¹⁰ M, less than about 1×10⁻¹¹ M, less than about 5×10⁻¹¹ M, less than about 1×10⁻¹² M, or less than about 5×10⁻¹² M. In some embodiments, an antibody or antigen-binding fragment of the present disclosure binds to SARS-CoV-2 S protein RBD with a K_{D} of less than about 4.5×10⁻⁹ M, less than about 5×10⁻⁹ M, less than about 1×10⁻¹⁰ M, less than about 5×10⁻¹⁰ M, less than about 1×10⁻¹¹ M, less than about 5×10⁻¹¹ M, less than about 1×10⁻¹² M, or less than about 5×10⁻¹² M.

In certain embodiments, an antibody of the present disclosure is capable of neutralizing infection by SARS-CoV-2. In certain embodiments, an antibody of the present disclosure is capable of neutralizing infection by two or more sarbecoviruses. As used herein, a “neutralizing antibody” is one that can neutralize, i.e., prevent, inhibit, reduce, impede, or interfere with, the ability of a pathogen to initiate and/or perpetuate an infection in a host. The terms “neutralizing antibody” and “an antibody that neutralizes” or “antibodies that neutralize” are used interchangeably herein. In any of the presently disclosed embodiments, the antibody or antigen-binding fragment is capable of preventing and/or neutralizing a SARS-CoV-2 infection (or infection by another sarbecovirus) in an in vitro model of infection and/or in an in vivo animal model of infection and/or in a human. In some embodiments, an antibody or antigen-binding fragment of the present disclosure is capable of neutralizing a SARS-CoV-2 infection (or infection by another sarbecovirus) or a virus pseudotyped with an IC50 of about 16 to about 20 μg/ml. In some embodiments, an antibody or antigen-binding fragment is capable of neutralizing a SARS-CoV-2 infection (or infection by another sarbecovirus), or a virus pseudotyped with SARS-CoV-2, with an IC50 of about 3 to about 4 μg/ml. In any of the presently disclosed embodiments, an antibody or antigen-binding fragment is capable of neutralizing a SARS-CoV-2 infection (or infection by another sarbecovirus), or a virus pseudotyped with SARS-CoV-2, with an IC50, an IC80, an IC90, and/or an IC95 as shown in Table 4.

In some embodiments, an antibody or antigen-binding fragment, or a composition comprising two or more antibodies or antigen-binding fragments, of the present disclosure is capable of neutralizing a SARS-CoV-2 infection, or a virus pseudotyped with SARS-CoV-2, with an IC50 of about 0.8 to about 0.9 μg/ml. In some embodiments, an antibody or antigen-binding fragment, or a composition comprising two or more antibodies or antigen-binding fragments, of the present disclosure is capable of neutralizing a SARS-CoV-2 infection, or a virus pseudotyped with SARS-CoV-2, with an IC50 of about 0.5 to about 0.6 μg/ml. In some embodiments, an antibody or antigen-binding fragment, or a composition comprising two or more antibodies or antigen-binding fragments, of the present disclosure is capable of neutralizing a SARS-CoV-2 infection, or a virus pseudotyped with SARS-CoV-2, with an IC50 of about 0.1 to about 0.2 μg/ml.

In certain embodiments, the antibody or antigen-binding fragment (i) recognizes an epitope in the ACE2 receptor binding motif (RBM, SEQ ID NO.:5) of SARS-CoV-2; (ii) is capable of blocking an interaction between SARS-CoV-2 and ACE2; (ii) is capable of binding to SARS-CoV-2 S protein with greater avidity than to SARS-CoV S protein; (iv) recognizes an epitope that is conserved in the ACE2 RBM of SARS-CoV-2 and in an ACE2 RBM of SARS-CoV; (v) is cross-reactive against SARS-CoV-2 and SARS-CoV; (vi) recognizes an epitope in the SARS-CoV-2 surface glycoprotein that is not in the ACE2 RBM; or (vii) any combination of (i)-(vi).

In certain embodiments, the antibody or antigen-binding fragment (i) recognizes an epitope in the Spike protein of two or more sarbecoviruses; (ii) is capable of blocking an interaction between the Spike protein of one or more sarbecoviruses and a cell surface receptor; (iii) recognizes an epitope that is conserved in the Spike protein of two or more sarbecoviruses; (iv) is cross-reactive against two or more sarbecoviruses; or (v) any combination of (i)-(iv).

In some embodiments, an antibody or antigen-binding fragment thereof is capable of capable of inhibiting an interaction between: (i) SARS-CoV-2 and a human DC-SIGN; (ii) SARS-CoV-2 and a human L-SIGN; (iii) SARS-CoV-2 and a human SIGLEC-1; or (iv) any combination of (i)-(iii). As disclosed herein, DC-SIGN, L-SIGN, and SIGLEC-1 can be involved in a SARS-CoV-2 infection, in roles comprising those of attachment receptors. Inhibiting an interaction between SARS-CoV-2 and DC-SIGN, L-SIGN, and/or SIGLEC-1 can, in some contexts, neutralize infection by the SARS-CoV-2.

In some embodiments, an antibody or antigen-binding fragment thereof is capable of binding to a surface glycoprotein of: (i) a SARS-CoV-2 Wuhan-Hu-1 (SEQ ID NO.:3); (ii) a SARS-CoV-2 B.1.1.7; and/or (iii) a SARS-CoV-2 B.1.351.

Terms understood by those in the art of antibody technology are each given the meaning acquired in the art, unless expressly defined differently herein. For example, the term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

The terms “V_{L}” or “VL” and “V_{H}” or “VH” refer to the variable binding region from an antibody light chain and an antibody heavy chain, respectively. In certain embodiments, a VL is a kappa (κ) class (also “VK” herein). In certain embodiments, a VL is a lambda (λ) class. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary structure by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs.

As used herein, a “variant” of a CDR refers to a functional variant of a CDR sequence having up to 1-3 amino acid substitutions (e.g., conservative or non-conservative substitutions), deletions, or combinations thereof.

Numbering of CDR and framework regions may be according to any known method or scheme, such as the Kabat, Chothia, EU, IMGT, and AHo numbering schemes (see, e.g., Kabat et al., “Sequences of Proteins of Immunological Interest,” US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Pluckthun, J. Mol. Bio. 309:657-670 (2001)). Equivalent residue positions can be annotated and for different molecules to be compared using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme. In certain embodiments, an antibody or antigen-binding fragment is provided that comprises CDRs of a VH sequence according to any one of SEQ ID NOs.: 22, 30, 32, 34, 35, 37, 45, 47, 49, 50, 52, 54, 62, 64, 66, 68, 69, 71, 81, 91, 101, 111, 121, 135, 145, 155, 158, 180, 190, 200, 210, 220, 232, 242, 252, 253, 255, 256, 258, 260, 262, 264, 266, 267, 270, 272, 274, 276, 278, 280, 284, 286, 288, 291, 292, 295, 297, 298, 300, 304, 314, 348, 358, 368, 378, 388, 398, 408, 418, 428, 432, 434, 437, 446, 448, 458, 459, and 460, and in a VL sequence according to any one of SEQ ID NOs.: 26, 41, 58, 75, 85, 95, 105, 115, 125, 139, 149, 184, 194, 204, 214, 224, 230, 236, 246, 282, 302, 308, 319, 352, 362, 372, 382, 392, 402, 412, 422, 439, 442, 443, 444, and 445, according to any known CDR numbering method, such as, for example, the Kabat, Chothia, EU, IMGT, Martin (Enhanced Chothia), Contact, or AHo numbering method. In certain embodiments, CDRs are according to the antibody numbering method developed by the Chemical Computing Group (CCG); e.g., using Molecular Operating Environment (MOE) software (www.chemcomp.com).

In certain embodiments, an antibody or an antigen-binding fragment is provided that comprises a heavy chain variable domain (VH) comprising a CDRH1, a CDRH2, and a CDRH3, and a light chain variable domain (VL) comprising a CDRL1, a CDRL2, and a CDRL3, wherein: (i) the CDRH1 comprises or consists of the amino acid sequence according to any one of SEQ ID NOs.: 23, 33, 38, 46, 53, 55, 63, 70, 72, 83, 93, 103, 113, 123, 137, 147, 160, 166, 181, 191, 201, 211, 221, 233, 243, 268, 305, 315, 325, 330, 335, 349, 359, 369, 379, 389, 399, 409, 419, or 449, or a sequence variant thereof comprising one, two, or three acid substitutions, one or more of which substitutions is optionally a conservative substitution and/or is a substitution to a germline-encoded amino acid; (ii) the CDRH2 comprises or consists of the amino acid sequence according to any one of SEQ ID NOs.: 24, 31, 36, 39, 48, 51, 56, 65, 67, 73, 83, 93, 103, 113, 123, 137, 147, 161, 167, 182, 192, 202, 212, 222, 234, 244, 263, 269, 285, 287, 289, 293, 299, 301, 306, 316, 326, 331, 336, 350, 360, 370, 380, 390,400, 410, 420, 447, or 457, or a sequence variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence according to any one of SEQ ID NOs.: 25, 40, 57, 74, 84, 94, 104, 114, 124, 138, 148, 156, 162, 168, 183, 193, 203, 213, 223, 235, 245, 254, 257, 259, 261, 265, 271, 273, 275, 277, 279, 281, 290, 294, 296, 307, 317, 324, 327, 332, 337, 351, 361, 371, 381, 391, 401, 411, 421, or 435, or a sequence variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence according to any one of SEQ ID NOs.: 27, 42, 59, 76, 86, 96, 106, 116, 126, 140, 150, 163, 169, 185, 195, 205, 215, 225, 237, 247, 309, 319, 328, 333, 338, 353, 363, 373, 383, 393, 403, 413, or 423, or a sequence variant thereof comprising one, two, or three amino acid substitutions, one or more of which substitutions is optionally a conservative substitution and/or is a substitution to a germline-encoded amino acid; (v) the CDRL2 comprises or consists of the amino acid sequence according to any one of SEQ ID NOs.: 28, 43, 60, 77, 87, 97, 107, 117, 127, 141, 151, 164, 170, 186, 106, 206, 216, 226, 238, 248, 310, 320, 329, 334, 339, 354, 364, 374, 384, 394, 404, 414, 424, or 440, or a sequence variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence according to any one of SEQ ID NOs.: 29, 44, 61, 78, 88, 98, 108, 118, 128, 142, 152, 165, 171, 187, 197, 207, 217, 227, 239, 249, 283, 303, 311, 321, 355, 365, 375, 385, 395, 405, 415, or 425, or a sequence variant thereof comprising having one, two, or three amino acid substitutions, one or more of which substitutions is optionally a conservative substitution and/or is a substitution to a germline-encoded amino acid, wherein the antibody or antigen binding fragment is capable of binding to a surface glycoprotein of SARS-CoV-2 expressed on a cell surface of a host cell.

In any of the presently disclosed embodiments, the antibody or antigen-binding fragment is capable of preventing and/or neutralizing a SARS-CoV-2 infection in an in vitro model of infection and/or in an in vivo animal model of infection and/or in a human.

In any of the presently disclosed embodiments, the antibody or antigen-binding fragment comprises CDRH1, CDRH2, CDRH3, CDRL1, CDRL2, and CDRL3 amino acid sequences according to SEQ ID NOs.: (i) 23-25 and 27-29, respectively; (ii) 23 or 160, 31, 25 or 162, and 27-29 or 163-165, respectively; (iii) 33, 24 or 161, 25 or 162, and 27-29 or 163-165, respectively; (iv) 33, 31, 25 or 162, and 27-29 or 163-165, respectively; (v) 33, 36, 25 or 162, and 27-29 or 163-165, respectively; (vi) 38-40 and 42-44, respectively; (vii) 46, 39 or 167, 40 or 168, and 42-44 or 169-171, respectively; (viii) 38 or 166, 48, 40 or 168 and 42-44 or 169-171, respectively; (ix) 46, 48, 40 or 168 and 42-44 or 169-171, respectively; (x) 46, 51, 40 or 168, and 42-44 or 169-171, respectively; (xi) 53, 48, 40 or 168, and 42-44 or 169-171, respectively; (xii) 55-57 and 59-61, respectively; (xiii) 63, 56, 57 and 59-61, respectively; (xiv) 55, 65, 57 and 59-61, respectively; (xv) 63, 67, 57, and 59-61, respectively; (xvi) 63, 65, 57 and 59-61, respectively; (xvii) 70, 65, 57, and 59-61, respectively; (xviii) 72-74 and 76-78, respectively; (xix) 82-84 and 86-88, respectively; (xx) 92-94 and 96-98, respectively; (xxi) 102-104, and 106-108, respectively; (xxii) 112-114 and 116-118, respectively; (xxiii) 122-124 and 126-128, respectively; (xxiv) 136-138 and 140-142, respectively; (xxv) 146-148 and 150-152, respectively; (xxvi) 112, 113, 156 and 116-118, respectively; (xxvii) 181-183 and 185-187, respectively; (xxviii) 191-193 and 195-197, respectively; (xxix) 201-203 and 205-207, respectively; (xxx) 211-213 and 215-217, respectively; (xxxi) 221-223 and 225-227, respectively; (xxxii) 233-235 and 237-239, respectively; (xxxiii) 243-245 and 247-249, respectively; (xxxiv) 211, 212, any one of 254, 257, 259, 261, or 324 and 215-217, respectively; (xxxv) any one of 221, 268, or 325, any one of 222, 263, 269, or 326, any one of 223, 265, 271, 273, or 327 and 225, 226 or 328, and 227 or 329, respectively; (xxxvi) 233 or 330, 234 or 331, any one of 235, 275, 277, 279, 281, or 332, any one of 237, 282, or 333, 238 or 334, and 239, respectively; (xxxvii) 243 or 335, any one of 244, 285, 287, 289, 293, 299, 301, or 336, any one of 245, 290, 294, 296, or 337, 247 or 338, 248 or 339, and 249 or 303, respectively; (xxxviii) 305-307 and 309-311, respectively; (xxxix) 315-317 and 319-321, respectively; (xxxx) 349-351 and 353-355, respectively; (xxxxi) 359-361 and 363-365, respectively; (xxxxii) 369-371 and 373-375, respectively; (xxxxiii) 379-381 and 383-385, respectively; (xxxxiv) 389-391 and 393-395, respectively; (xxxxv) 399, 400, 401 or 435, and 403, 404 or 440, and 405, respectively; (xxxxvi) 409-411 and 413-415, respectively; (xxxxvii) 419-421 and 423-425, respectively; (xxxxviii) 409, 447, 411, and 413-415, respectively; (xxxxix) 449, 410, 411, and 413-415, respectively; or (xxxxx) 449, 447, 411, and 413-415, respectively.

In certain embodiments, an antibody or an antigen-binding fragment of the present disclosure comprises a CDRH1, a CDRH2, a CDRH3, a CDRL1, a CDRL2, and a CDRL3, wherein each CDR is independently selected from a corresponding CDR of SARS-CoV-2 S2H7-v1 mAb, SARS-CoV-2 S2H7-v2 mAb, SARS-CoV-2 S2H7-v3 mAb, SARS-CoV-2 S2H7-v4 mAb, SARS-CoV-2 S2H7-v5 mAb, SARS-CoV-2 S2H13-v1 mAb, SARS-CoV-2 S2H13-v2 mAb, SARS-CoV-2 S2H13-v3 mAb, SARS-CoV-2 S2H13-v4 mAb, or SARS-CoV-2 S2H13-v5 mAb SARS-CoV-2 S2H13-v6 mAb, SARS-CoV-2 S2H14-v1 mAb, SARS-CoV-2 S2H14-v2 mAb, SARS-CoV-2 S2H14-v3 mAb, SARS-CoV-2 S2H14-v4 mAb, SARS-CoV-2 S2H14-v5 mAb, SARS-CoV-2 S2-H14-v6 mAb, SARS-CoV-2 S2A4-v1 mAb, SARS-CoV-2 S2A5-v1mAb, SARS-CoV-2 S2A10-v1 mAb, SARS-CoV-2 S2A15-v1 mAb, SARS-CoV-2 S2A15-v2 mAb, SARS-CoV-2 S2-B2v1, SARS-CoV-2 S2B2-v2 mAb, SARS-CoV-2 S2F1-v1, SARS-CoV-2 S2H7-v1 mAb, SARS-CoV-2 S2R5-v1 mAb, SARS-CoV-2 S2R7-v1 mAb, SARS-CoV-2 S2N3-v1 mAb, SARS-CoV-2 S2N6-v1 mAb, SARS-CoV-2 S2X2-v1 mAb, SARS-CoV-2 S2D8-v1 mAb, SARS-CoV-2 S2D25-v1 mAb, SARS-CoV-2 S2D25-v2 mAb, SARS-CoV-2 S2D32-v1 mAb, SARS-CoV-2 S2D60-v1 mAb, SARS-CoV-2 S2X127-v1 mAb, SARS-CoV-2 S2X129-v1 mAb, SARS-CoV-2 S2X132-v1 mAb, SARS-CoV-2 S2X190-v1 mAb, SARS-CoV-2 S2X200 mAb, SARS-CoV-2 S2X227 mAb, SARS-CoV-2 S2X259 mAb, SARS-CoV-2 S2X259-v3 mAb, SARS-CoV-2 S2X259-v4 mAb, SARS-CoV-2 S2X259-v5 mAb, SARS-CoV-2 S2X259-v6 mAb, SARS-CoV-2 S2X259-v7 mAb, SARS-CoV-2 S2X259-v8 mAb, SARS-CoV-2 S2X288 mAb, Antibody 407_10_1_v2, Antibody 407_10_2_v2, Antibody 407_10_2_v3, Antibody 407_10_2_v4, or Antibody 407_10_2_v5, as provided in Table 3. That is, all combinations of CDRs from SARS-CoV-2 mAbs and the variant sequences thereof provided in Table 3 are contemplated.

Exemplary antibodies of the present disclosure include antibody S2X259 and engineered variants thereof. In particular embodiments, an antibody or antigen-binding fragment comprises a CDRH1, a CDRH2, a CDRH3, a CDRL1, a CDRL2, and a CDRL3 selected from any of the CDRH1, CDRH2, CDRH3, CDRL1, CDRL2, and CDRL3 amino acid sequences (respectively) provided in Table 1. In some embodiments, an antibody or antigen-binding fragment comprises: a CDRH1, a CDRH2, and a CDRH3 of the VH amino acid sequence set forth in any one of SEQ ID NOs.:408, 428, 446, 448, 458, 459, and 460; and a CDRL1, a CDRL2, and a CDRL3 of the VL amino acid sequence set forth in any one of SEQ ID NOs.:412, 442, 443, 444, and 445 (i.e., according to any CDR numbering or determination method known in the art, such as IMGT, Kabat, Chothia, AHo, North, Contact, CCG, EU, or Martin (Enhanced Chothia)). In further embodiments, the antibody or antigen-binding fragment comprises a VH having at least 85% identity (i.e., 85%, 86, 87, 88, 89, 90, 91, 92, 93, 94, 95, 96, 97, 98, 99, or 100%) identity to a VH amino acid sequence provided in Table 1 and/or a VL having at least 85% identity (i.e., 85%, 86, 87, 88, 89, 90, 91, 92, 93, 94, 95, 96, 97, 98, 99, or 100%) identity to a VL amino acid sequence provided in Table 1. In still further embodiments, the antibody or antigen-binding fragment comprises a VH having at least 90% identity identity to a VH amino acid sequence provided in Table 1 and/or a VL having at least 90% identity to a VL amino acid sequence provided in Table 1. In still further embodiments, the antibody or antigen-binding fragment comprises a VH having at least 95% identity identity to a VH amino acid sequence provided in Table 1 and/or a VL having at least 95% identity to a VL amino acid sequence provided in Table 1. In still further embodiments, the antibody or antigen-binding fragment comprises a VH having at least 99% identity identity to a VH amino acid sequence provided in Table 1 and/or a VL having at least 99% identity to a VL amino acid sequence provided in Table 1. In some embodiments, the antibody or antigen-binding fragment comprises a VH amino acid sequence selected from the VH amino acid sequences provided in Table 1 and a VL amino acid sequence selected from the VL amino acid sequence provided in Table 1.

TABLE 1
CDR and Variable Region Amino Acid Sequences of Certain S2X259
Antibodies.

| | |
|---|---|
| CDRH1 | GGIFNTYT (SEQ ID NO.: 409); GGIFSTYT (SEQ ID NO.: 449) |
| CDRH2 | IILMSGMA (SEQ ID NO.: 410); IILISGIA (SEQ ID NO.: 447); |
| | IILMSGKA (SEQ ID NO.: 457) |
| CDRH3 | ARGENGNYYGWGDDDAFDI (SEQ ID NO.: 411) |
| VH | QVQLVQSGAEVKKPGSSVKVSCKASGGIFNTYTISWVRQAPGQ |
| | GLEWMGRIILMSGMANYAQKIQGRVTITADKSTSTAYMELTSL |
| | RSDDTAVYYCARGENGNYYGWGDDDAFDISGQGTLVTVYS |
| | (SEQ ID NO.: 408) |
| | QVQLVQSGAEVKKPGSSVKVSCKASGGIFNTYTISWVRQAPGQ |
| | GLEWMGRIILMSGMANYAQKIQGRVTITADKSTSTAYMELTSL |
| | RSDDTAVYYCARGENGNYYGWGDDDAFDIWGQGTLVTVYS |
| | (SEQ ID NO.: 428) |
| | QVQLVQSGAEVKKPGSSVKVSCKASGGIFNTYTISWVRQAPGQ |
| | GLEWMGRIILISGIANYAQKIQGRVTITADKSTSTAYMELTSLRS |
| | DDTAVYYCARGENGNYYGWGDDDAFDISGQGTLVTVYS (SEQ |
| | ID NO.: 446) |
| | QVQLVQSGAEVKKPGSSVKVSCKASGGIFSTYTISWVRQAPGQ |
| | GLEWMGRIILMSGMANYAQKIQGRVTITADKSTSTAYMELTSL |
| | RSDDTAVYYCARGFNGNYYGWGDDDAFDISGQGTLVTVYS |
| | (SEQ ID NO.: 448) |
| | QVQLVQSGAEVKKPGSSVKVSCKASGGIFNTYTISWVRQAPGQ |
| | GLEWMGRIILMSGMANYAQKIQGRVTITADKSTSTAYMELTSL |
| | RSDDTAVYYCARGENGNYYGWGDDDAFDIWGQGTLVTVSS |
| | (SEQ ID NO.: 458) |
| | QVQLVQSGAEVKKPGSSVKVSCKASGGIFNTYTISWVRQAPGQ |
| | GLEWMGRIILMSGKANYAQKIQGRVTITADKSTSTAYMELTSL |
| | RSDDTAVYYCARGENGNYYGWGDDDAFDIWGQGTLVTVSS |
| | (SEQ ID NO.: 459) |
| | QVQLVQSGAEVKKPGSSVKVSCKASGGIFNTYTISWVRQAPGQ |
| | GLEWMGRIILISGIANYAQKIQGRVTITADKSTSTAYMELTSLRS |
| | DDTAVYYCARGENGNYYGWGDDDAFDIWGQGTLVTVSS |
| | (SEQ ID NO.: 460) |
| CDRL1 | NSNIGAGYD (SEQ ID NO.: 413) |
| CDRL2 | GNS (SEQ ID NO.: 414) |
| CDRL3 | QSYDSSLSGPNWV (SEQ ID NO.: 415) |
| VL | QTVLTQPPSVSGAPGQRVTISCTGSNSNIGAGYDVHWYQQLPGT |
| | APKLLICGNSNRPSGVPDRFSGSKSGTSASLAITGLQAEDEADYY |
| | CQSYDSSLSGPNWVFGGGTKLTVL (SEQ ID NO.: 412) |
| | QTVLTQPPSVSGAPGQRVTISCTGSNSNIGAGYDVHWYQQLPGT |
| | APKLLISGNSNRPSGVPDRFSGSKSGTSASLAITGLQAEDEADYY |
| | CQSYDSSLSGPNWVFGGGTKLTVL (SEQ ID NO.: 442) |
| | QTVLTQPPSVSGAPGQRVTISCTGSNSNIGAGYDVHWYQQLPGT |
| | APKLLIAGNSNRPSGVPDRFSGSKSGTSASLAITGLQAEDEADYY |
| | CQSYDSSLSGPNWVFGGGTKLTVL (SEQ ID NO.: 443) |
| | QTVLTQPPSVSGAPGQRVTISCTGSNSNIGAGYDVHWYQQLPGT |
| | APKLLIYGNSNRPSGVPDRFSGSKSGTSASLAITGLQAEDEADYY |
| | CQSYDSSLSGPNWVFGGGTKLTVL (SEQ ID NO.: 444) |
| | QTVLTQPPSVSGAPGQRVTISCTGSNSNIGAGYDVHWYQQLPGT |
| | APKLLIVGNSNRPSGVPDRFSGSKSGTSASLAITGLQAEDEADYY |
| | CQSYDSSLSGPNWVFGGGTKLTVL (SEQ ID NO.: 445) |

In particular embodiments, the antibody or antigen-binding fragment comprises the CDRH1, CDRH2, and CDRH3 amino acid sequences set forth in SEQ ID NOs.:409 or 449, 410, 447, or 457, and 411, respectively, and the CDRL1, CDRL2, and CDRL3 amino acid sequences set forth in SEQ ID NOs.:413, 414, and 415, respectively. In some embodiments, the antibody or antigen-binding fragment comprises the CDRH1, CDRH2, CDRH3, CDRL1, CDRL2, and CDRL3 amino acid sequences set forth in: (a) SEQ ID NOs.: 409, 410, 411, 413, 414, and 415, respectively; (b) SEQ ID NOs.: 409, 447, 411, 413, 414, and 415, respectively; (c) SEQ ID NOs.: 409, 457, 411, 413, 414, and 415, respectively; (d) SEQ ID NOs.: 449, 410, 411, 413, 414, and 415, respectively; (e) SEQ ID NOs.: 449, 447, 411, 413, 414, and 415, respectively; or (f) SEQ ID NOs.: 449, 457, 411, 413, 414, and 415, respectively.

In further embodiments, the VH and the VL have at least 85% identity to, at least 90% identity to, at least 95% identity to, at least 97% identity to, at least 99% identity to, or comprise or consist of, the amino acid sequences set forth in SEQ ID NOs.: (i) 408 and 412, respectively; (ii) 408 and 442, respectively; (iii) 408 and 443, respectively; (iv) 408 and 444, respectively; (v) 408 and 445, respectively; (vi) 428 and 412, respectively; (vii) 428 and 442, respectively; (viii) 428 and 443, respectively; (ix) 428 and 444, respectively; (x) 428 and 445, respectively; (xi) 446 and 412, respectively; (xii) 446 and 442, respectively; (xiii) 446 and 443, respectively; (xiv) 446 and 444, respectively; (xv) 446 and 445, respectively; (xvi) 448 and 412, respectively; (xvii) 448 and 442, respectively; (xviii) 448 and 443, respectively; (xix) 448 and 444, respectively; (xx) 448 and 445, respectively; (xxi) 458 and 412, respectively; (xxii) 458 and 442, respectively; (xxiii) 458 and 443, respectively; (xxiv) 458 and 444, respectively; (xxv) 458 and 445, respectively; (xxvi) 459 and 412, respectively; (xxvii) 459 and 442, respectively; (xxviii) 459 and 443, respectively; (xxix) 459 and 444, respectively; (xxx) 459 and 445, respectively; (xxxi) 460 and 412, respectively; (xxxii) 460 and 442, respectively; (xxxiii) 460 and 443, respectively; (xxxiv) 460 and 444, respectively; or (xxxv) 460 and 445, respectively.

Exemplary antibodies of the present disclosure also include antibody S2X227 and engineered variants thereof. In particular embodiments, an antibody or antigen-binding fragment comprises a CDRH1, a CDRH2, a CDRH3, a CDRL1, a CDRL2, and a CDRL3 selected from any of the CDRH1, CDRH2, CDRH3, CDRL1, CDRL2, and CDRL3 amino acid sequences (respectively) provided in Table 2.

In some embodiments, an antibody or antigen-binding fragment comprises: a CDRH1, a CDRH2, and a CDRH3 of the VH amino acid sequence set forth in any one of SEQ ID NOs.:398, 432, 434, and 437; and a CDRL1, a CDRL2, and a CDRL3 of the VL amino acid sequence set forth in any one of SEQ ID NOs.:402 and 439 (i.e., according to any CDR numbering or determination method known in the art, such as IMGT, Kabat, Chothia, AHo, North, Contact, CCG, EU, or Martin (Enhanced Chothia)).

In further embodiments, the antibody or antigen-binding fragment comprises a VH having at least 85% identity (i.e., 85%, 86, 87, 88, 89, 90, 91, 92, 93, 94, 95, 96, 97, 98, 99, or 100%) identity to a VH amino acid sequence provided in Table 2 and/or a VL having at least 85% identity (i.e., 85%, 86, 87, 88, 89, 90, 91, 92, 93, 94, 95, 96, 97, 98, 99, or 100%) identity to a VL amino acid sequence provided in Table 2. In still further embodiments, the antibody or antigen-binding fragment comprises a VH having at least 90% identity identity to a VH amino acid sequence provided in Table 2 and/or a VL having at least 90% identity to a VL amino acid sequence provided in Table 2. In still further embodiments, the antibody or antigen-binding fragment comprises a VH having at least 95% identity identity to a VH amino acid sequence provided in Table 2 and/or a VL having at least 95% identity to a VL amino acid sequence provided in Table 2. In still further embodiments, the antibody or antigen-binding fragment comprises a VH having at least 99% identity identity to a VH amino acid sequence provided in Table 2 and/or a VL having at least 99% identity to a VL amino acid sequence provided in Table 2. In some embodiments, the antibody or antigen-binding fragment comprises a VH amino acid sequence selected from the VH amino acid sequences provided in Table 2 and a VL amino acid sequence selected from the VL amino acid sequence provided in Table 2.

TABLE 2
CDR and Variable Region Amino Acid Sequences of Certain S2X227
Antibodies

| | |
|---|---|
| CDRH1 | GYTFTSYY (SEQ ID NO.: 399) |
| CDRH2 | INPGGVST (SEQ ID NO.: 400) |
| CDRH3 | ARSIAVFWGDAFDI (SEQ ID NO.: 401); |
| | ARSIAVFFGDAFDI (SEQ ID NO.: 435) |
| VH | EVQLVQSGAEVKKPGASVKVSCKASGYTFTSYYMHWVRQAPG |
| | QGLEWMGIINPGGVSTTYAHYAQKFQGRVTMTRDTSTSTVYM |
| | ELSSLRSEDTAVYYCARSIAVFWGDAFDIWGQGTMVTVSS |
| | (SEQ ID NO.: 398) |
| | EVQLVQSGAEVKKPGASVKVSCKASGYTFTSYYIHWVRQAPGQ |
| | GLEWMGIINPGGVSTTYAHYAQKFQGRVTMTRDTSTSTVYM |
| | ELSSLRSEDTAVYYCARSIAVFWGDAFDIWGQGTMVTVSS |
| | (SEQ ID NO.: 432) |
| | EVQLVQSGAEVKKPGASVKVSCKASGYTFTSYYMHWVRQAPG |
| | QGLEWMGIINPGGVSTTYAHYAQKFQGRVTMTRDTSTSTVYM |
| | ELSSLRSEDTAVYYCARSIAVFFGDAFDIWGQGTMVTVSS |
| | (SEQ ID NO.: 434) |
| | EVQLVQSGAEVKKPGASVKVSCKASGYTFTSYYIHWVRQAPGQ |
| | GLEWMGIINPGGVSTTYAHYAQKFQGRVTMTRDTSTSTVYM |
| | ELSSLRSEDTAVYYCARSIAVFFGDAFDIWGQGTMVTVSS |
| | (SEQ ID NO.: 437) |
| CDRL1 | QSVLYSSNNKNY (SEQ ID NO.: 403) |
| CDRL2 | WAS (SEQ ID NO.: 404); |
| | FAS (SEQ ID NO.: 440) |
| CDRL3 | QQYSSSPLT (SEQ ID NO.: 405) |
| VL | DIQMTQSPDSLAVSLGERATINCKSSQSVLYSSNNKNYLAWYQQ |
| | KPGQPPKLLIYWASTRESGVPDRFSGSGSGTDFTLTISSLQAED |
| | VAVYYCQQYSSSPLTFGGGTKVEIK |
| | (SEQ ID NO.: 402) |
| | DIQMTQSPDSLAVSLGERATINCKSSQSVLYSSNNKNYLAWYQQ |
| | KPGQPPKLLIYFASTRESGVPDRFSGSGSGTDFTLTISSLQAED |
| | VAVYYCQQYSSSPLTFGGGTKVEIK |
| | (SEQ ID NO.: 439) |

In particular embodiments, the antibody or antigen-binding fragment comprises the CDRH1, CDRH2, and CDRH3 amino acid sequences set forth in SEQ ID NOs.:399, 400, and 401 or 435, respectively, and the CDRL1, CDRL2, and CDRL3 amino acid sequences set forth in SEQ ID NOs.:403, 404 or 440, and 405, respectively. In some embodiments, the antibody or antigen-binding fragment comprises the CDRH1, CDRH2, CDRH3, CDRL1, CDRL2, and CDRL3 amino acid sequences set forth in: (a) SEQ ID NOs.: 399, 400, 401, 403, 404, and 405, respectively; (b) SEQ ID NOs.: 399, 400, 435, 403, 404, and 405, respectively; (c) SEQ ID NOs.: 399, 400, 401, 403, 440, and 405, respectively; or (d) SEQ ID NOs.: 399, 400, 435, 403, 440, and 405, respectively.

In further embodiments, the VH and the VL have at least 85% identity to, at least 90% identity to, at least 95% identity, at least 97% identity to, at least 99% identity to, or comprise or consist of, the amino acid sequences set forth in SEQ ID NOs.: (i) 398 and 402, respectively; (ii) 398 and 439, respectively; (iii) 432 and 402, respectively; (iv) 432 and 439, respectively; (v) 434 and 402, respectively; (vi) 434 and 439, respectively; (vii) 437 and 402, respectively; or (viii) 437 and 439, respectively.

The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). The Fc region of an antibody heavy chain is described further herein. In any of the presently disclosed embodiments, an antibody or antigen-binding fragment of the present disclosure comprises any one or more of CL, a CH1, a CH2, and a CH3. In certain embodiments, a CL comprises an amino acid sequence having 90%, 91%, 92%, 93%, 94%, 95%, 96%, 975, 98%, 99%, or 100% identity to the amino acid sequence of SEQ ID NO.:6 or SEQ ID NO.: 7. In certain embodiments, a CH1-CH2-CH3 comprises an amino acid sequence having 90%, 91%, 92%, 93%, 94%, 95%, 96%, 975, 98%, 99%, or 100% identity to the amino acid sequence of SEQ ID NO.:6 or SEQ ID NO.:7. It will be understood that, for example, production in a mammalian cell line can remove one or more C-terminal lysine of an antibody heavy chain (see, e.g., Liu et al. mAbs 6(5):1145-1154 (2014)). Accordingly, an antibody or antigen-binding fragment of the present disclosure can comprise a heavy chain, a CH1-CH3, a CH3, or an Fc polypeptide wherein a C-terminal lysine residue is present or is absent; in other words, encompassed are embodiments where the C-terminal residue of a heavy chain, a CH1-CH3, or an Fc polypeptide is not a lysine, and embodiments where a lysine is the C-terminal residue. In certain embodiments, a composition comprises a plurality of an antibody and/or an antigen-binding fragment of the present disclosure, wherein one or more antibody or antigen-binding fragment does not comprise a lysine residue at the C-terminal end of the heavy chain, CH1-CH3, or Fc polypeptide, and wherein one or more antibody or antigen-binding fragment comprises a lysine residue at the C-terminal end of the heavy chain, CH1-CH3, or Fc polypeptide.

A “Fab” (fragment antigen binding) is the part of an antibody that binds to antigens and includes the variable region and CHI of the heavy chain linked to the light chain via an inter-chain disulfide bond. Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Both the Fab and F(ab′)2 are examples of “antigen-binding fragments.” Fab′ fragments differ from Fab fragments by having additional few residues at the carboxy terminus of the CHI domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

Fab fragments may be joined, e.g., by a peptide linker, to form a single chain Fab, also referred to herein as “scFab”. In these embodiments, an inter-chain disulfide bond that is present in a native Fab may not be present, and the linker serves in full or in part to link or connect the Fab fragments in a single polypeptide chain. A heavy chain-derived Fab fragment (e.g., comprising, consisting of, or consisting essentially of VH+CH1, or “Fd”) and a light chain-derived Fab fragment (e.g., comprising, consisting of, or consisting essentially of VL+CL) may be linked in any arrangement to form a scFab. For example, a scFab may be arranged, in N-terminal to C-terminal direction, according to (heavy chain Fab fragment—linker—light chain Fab fragment) or (light chain Fab fragment—linker—heavy chain Fab fragment). Peptide linkers and exemplary linker sequences for use in scFabs are discussed in further detail herein.

“Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the VH and VL antibody domains connected into a single polypeptide chain. In some embodiments, the scFv polypeptide comprises a polypeptide linker disposed between and linking the VH and VL domains that enables the scFv to retain or form the desired structure for antigen binding. Such a peptide linker can be incorporated into a fusion polypeptide using standard techniques well known in the art. For a review of scFv, see Pluckthun in The Pharmacology of Monoclonal Antibodies, vol. 113, Rosenburg and Moore eds., Springer-Verlag, New York, pp. 269-315 (1994); Borrebaeck 1995, infra. In certain embodiments, the antibody or antigen-binding fragment comprises a scFv comprising a VH domain, a VL domain, and a peptide linker linking the VH domain to the VL domain. In particular embodiments, a scFv comprises a VH domain linked to a VL domain by a peptide linker, which can be in a VH-linker-VL orientation or in a VL-linker-VH orientation. Any scFv of the present disclosure may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C). Alternatively, in some embodiments, a linker may be linked to an N-terminal portion or end of the VH domain, the VL domain, or both.

Peptide linker sequences may be chosen, for example, based on: (1) their ability to adopt a flexible extended conformation; (2) their inability or lack of ability to adopt a secondary structure that could interact with functional epitopes on the first and second polypeptides and/or on a target molecule; and/or (3) the lack or relative lack of hydrophobic or charged residues that might react with the polypeptides and/or target molecule. Other considerations regarding linker design (e.g., length) can include the conformation or range of conformations in which the VH and VL can form a functional antigen-binding site. In certain embodiments, peptide linker sequences contain, for example, Gly, Asn and Ser residues. Other near neutral amino acids, such as Thr and Ala, may also be included in a linker sequence. Other amino acid sequences which may be usefully employed as linker include those disclosed in Maratea et al., Gene 40:39 46 (1985); Murphy et al., Proc. Natl. Acad. Sci. USA 83:8258 8262 (1986); U.S. Pat. Nos. 4,935,233, and 4,751,180. Other illustrative and non-limiting examples of linkers may include, for example, Glu-Gly-Lys-Ser-Ser-Gly-Ser-Gly-Ser-Glu-Ser-Lys-Val-Asp (SEQ ID NO: 19) (Chaudhary et al., Proc. Natl. Acad. Sci. USA 87:1066-1070 (1990)) and Lys-Glu-Ser-Gly-Ser-Val-Ser-Ser-Glu-Gln-Leu-Ala-Gln-Phe-Arg-Ser-Leu-Asp (SEQ ID NO: 20) (Bird et al., Science 242:423-426 (1988)) and the pentamer Gly-Gly-Gly-Gly-Ser (SEQ ID NO: 21) when present in a single iteration or repeated 1 to 5 or more times, or more; see, e.g., SEQ ID NO: 17. Any suitable linker may be used, and in general can be about 3, 4, 5, 6, 7, 8, 9, 10, 11, 12, 13, 14, 15, 16, 17, 18, 19, 20, 21, 22, 15 23, 24, 25, 26, 27, 28, 29, 30, 40, 50, 60, 70, 80, 90, 100 amino acids in length, or less than about 200 amino acids in length, and will preferably comprise a flexible structure (can provide flexibility and room for conformational movement between two regions, domains, motifs, fragments, or modules connected by the linker), and will preferably be biologically inert and/or have a low risk of immunogenicity in a human. Exemplary linkers include those comprising or consisting of the amino acid sequence set forth in any one or more of SEQ ID NOs: 10-21. In certain embodiments, the linker comprises or consists of an amino acid sequence having at least 75% (i.e., at least about 75%, 80%, 85%, 90%, 91%, 92%, 93%, 94%, 95%, 96%, 97%, 98%, 99%, or more) identity to the amino acid sequence set forth in any one of SEQ ID NOs: 10-21. scFv can be constructed using any combination of the VH and VL sequences or any combination of the CDRH1, CDRH2, CDRH3, CDRL1, CDRL2, and CDRL3 sequences disclosed herein.

In some embodiments, linker sequences are not required; for example, when the first and second polypeptides have non-essential N-terminal amino acid regions that can be used to separate the functional domains and prevent steric interference.

During antibody development, DNA in the germline variable (V), joining (J), and diversity (D) gene loci may be rearranged and insertions and/or deletions of nucleotides in the coding sequence may occur. Somatic mutations may be encoded by the resultant sequence, and can be identified by reference to a corresponding known germline sequence. In some contexts, somatic mutations that are not critical to a desired property of the antibody (e.g., binding to a SARS-CoV-2 antigen), or that confer an undesirable property upon the antibody (e.g., an increased risk of immunogenicity in a subject administered the antibody), or both, may be replaced by the corresponding germline-encoded amino acid, or by a different amino acid, so that a desirable property of the antibody is improved or maintained and the undesirable property of the antibody is reduced or abrogated. Thus, in some embodiments, the antibody or antigen-binding fragment of the present disclosure comprises at least one more germline-encoded amino acid in a variable region as compared to a parent antibody or antigen-binding fragment, provided that the parent antibody or antigen binding fragment comprises one or more somatic mutations. Variable region and CDR amino acid sequences of exemplary anti-SARS-CoV-2 antibodies of the present disclosure are provided in Tables 1, 2, and 3 herein.

In certain embodiments, an antibody or antigen-binding fragment comprises an amino acid modification (e.g., a substitution mutation) to remove an undesired risk of oxidation, deamidation, and/or isomerization.

Also provided herein are variant antibodies that comprise one or more amino acid alterations in a variable region (e.g., VH, VL, framework or CDR) as compared to a presently disclosed antibody, wherein the variant antibody is capable of binding to a SARS-CoV-2 antigen.

In certain embodiments, the VH comprises or consists of an amino acid sequence having at least 85% (i.e., 85%, 86, 87, 88, 89, 90, 91, 92, 93, 94, 95, 96, 97, 98, 99, or 100%) identity to the amino acid sequence according to any one of SEQ ID NOs.: 22, 30, 32, 34, 35, 37, 45, 47, 49, 50, 52, 54, 62, 64, 66, 68, 69, 71, 81, 91, 101, 111, 121, 135, 145, 155, 158, 180, 190, 200, 210, 220, 232, 242, 252, 253, 255,256, 258, 260, 262, 264, 266, 267, 270, 272, 274, 276, 278, 280, 284, 286, 288, 291, 292, 295, 297, 298, 300, 304, 314, 348, 358, 368, 378, 388, 398, 408, 418, 428, 432, 434, 437, 446, 448, 458, 459, and 460, wherein the variation is optionally limited to one or more framework regions and/or the variation comprises one or more substitution to a germline-encoded amino acid; and/or (ii) the VL comprises or consists of an amino acid sequence having at least 85% (i.e., 85%, 86, 87, 88, 89, 90, 91, 92, 93, 94, 95, 96, 97, 98, 99, or 100%) identity to the amino acid sequence according to any one of SEQ ID NOs.: 26, 41, 58, 75, 85, 95, 105, 115, 125, 139, 149, 184, 194, 204, 214, 224, 230, 236, 246, 282, 302, 308, 319, 352, 362, 372, 382, 392, 402, 412, 422, 439, 442, 443, 444, and 445, wherein the variation is optionally limited to one or more framework regions and/or the variation comprises one or more substitution to a germline-encoded amino acid.

In certain embodiments, the VH comprises or consists of any VH amino acid sequence set forth in Table 1, 2, or 3, and the VL comprises or consists of any VL amino acid sequence set forth in Table 1, 2, or 3. In particular embodiments, the VH and the VL comprise or consist of the amino acid sequences according to SEQ ID NOs.: (i) 22 and 26, respectively; (ii) 30 and 26, respectively; (iii) 32 and 26, respectively; (iv) 34 and 26, respectively; (v) 35 and 26, respectively; (vi) 37 and 41, respectively; (vii) 45 and 41, respectively; (viii) 47 and 41, respectively; (ix) 49 and 41, respectively; (x) 50 and 41, respectively; (xi) 52 and 41, respectively; (xii) 54 and 58, respectively; (xiii) 62 and 58, respectively; (xiv) 64 and 58, respectively; (xv) 66 and 58, respectively; (xvi) 68 and 58, respectively; (xvii) 69 and 58, respectively; (xviii) 71 and 75, respectively; (xix) 81 and 85, respectively; (xx) 91 and 95, respectively; (xxi) 101 or 158 and 105, respectively; (xxii) 111 or 155 and 115, respectively; (xxiii) 121 and 125, respectively; (xxiv) 135 and 139, respectively; (xxv) 145 and 149, respectively; (xxvi) 180 and 184, respectively; (xxvii) 190 and 194, respectively; (xxviii) 200 and 204, respectively; (xxix) 210 and 214; respectively; (xxx) 220 and 224, respectively; (xxxi) 220 and 230, respectively; (xxxii) 232 and 236, respectively; (xxxiii) 242 and 246, respectively; (xxxiv) any one of 252, 253, 255, 256, 258, or 260 and 214, respectively; (xxxv) any one of 262, 264, 266, 267, 270, or 272 and 224, respectively; (xxxvi) any one of 274, 276, 278, or 280 and 236 or 282, respectively; (xxxvii) any one of 284, 286, 288, 291, 292, 295, 297, 298, or 300 and 246 or 302, respectively; (xxxviii) 304 and 308, respectively; (xxxix) 314 and 318, respectively; (xxxx) 348 and 352, respectively; (xxxxi) 358 and 362, respectively; (xxxxii) 368 and 372, respectively; (xxxxiii) 378 and 382, respectively; (xxxxiv) 388 and 392, respectively; (xxxxv) 398 or 432 or 434 or 437 and 402 or 439, respectively; (xxxxvi) 408 or 428 and 412, respectively; (xxxxvii) 418 and 422, respectively; (xxxxviii) any one of 408, 446, and 448 and any one of 412, 442, 443, 444, and 445, respectively; or (xxxxix) any one of 408, 446, and 448 and any one of 412, 442, 443, 444, and 445, respectively, optionally according to (a) SEQ ID NOs.:408 and 412, respectively, (b) SEQ ID NOs.:408 and 442, respectively, (c) SEQ ID NOs.:408 and 443, respectively, (d) SEQ ID NOs.:408 and 444, respectively, (e) SEQ ID NOs.:408 and 445, respectively, (e) SEQ ID NOs.:446 and 412, respectively, (f) SEQ ID NOs.:446 and 442, respectively, (g) SEQ ID NOs.:446 and 443, respectively, (h) SEQ ID NOs.:446 and 444, respectively, (i) SEQ ID NOs.:446 and 448, respectively, (j) SEQ ID NOs.:448 and 412, respectively, (k) SEQ ID NOs.:448 and 442, respectively, (1) SEQ ID NOs.:448 and 443, respectively, (m) SEQ ID NOs.:448 and 444, respectively, or (n) SEQ ID NOs.:448 and 445, respectively.

In certain embodiments, an antibody or antigen-binding fragment of the present disclosure is monospecific (e.g., binds to a single epitope) or is multispecific (e.g., binds to multiple epitopes and/or target molecules). Antibodies and antigen binding fragments may be constructed in various formats. Exemplary antibody formats disclosed in Spiess et al., Mol. Immunol. 67(2):95 (2015), and in Brinkmann and Kontermann, mAbs 9(2):182-212 (2017), which formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, KX-bodies, orthogonal Fabs, DVD-Igs (e.g., U.S. Pat. No. 8,258,268, which formats are incorporated herein by reference in their entirety), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

In certain embodiments, the antibody or antigen-binding fragment comprises two or more of VH domains, two or more VL domains, or both (i.e., two or more VH domains and two or more VL domains). In particular embodiments, an antigen-binding fragment comprises the format (N-terminal to C-terminal direction) VH-linker-VL-linker-VH-linker-VL, wherein the two VH sequences can be the same or different and the two VL sequences can be the same or different. Such linked scFvs can include any combination of VH and VL domains arranged to bind to a given target, and in formats comprising two or more VH and/or two or more VL, one, two, or more different eptiopes or antigens may be bound. It will be appreciated that formats incorporating multiple antigen-binding domains may include VH and/or VL sequences in any combination or orientation. For example, the antigen-binding fragment can comprise the format VL-linker-VH-linker-VL-linker-VH, VH-linker-VL-linker-VL-linker-VH, or VL-linker-VH-linker-VH-linker-VL.

Monospecific or multispecific antibodies or antigen-binding fragments of the present disclosure constructed comprise any combination of the VH and VL sequences and/or any combination of the CDRH1, CDRH2, CDRH3, CDRL1, CDRL2, and CDRL3 sequences disclosed herein. A bispecific or multispecific antibody or antigen-binding fragment may, in some embodiments, comprise one, two, or more antigen-binding domains (e.g., a VH and a VL) of the instant disclosure. Two or more binding domains may be present that bind to the same or a different SARS-CoV-2 epitope, and a bispecific or multispecific antibody or antigen-binding fragment as provided herein can, in some embodiments, comprise a further SARS-CoV-2 binding domain, and/or can comprise a binding domain that binds to a different antigen or pathogen altogether.

In any of the presently disclosed embodiments, the antibody or antigen-binding fragment can be multispecific; e.g., bispecific, trispecific, or the like.

In certain embodiments, the antibody or antigen-binding fragment comprises: (i) a first VH and a first VL; and (ii) a second VH and a second VL, wherein the first VH and the second VH are different and each independently comprise an amino acid sequence having at least 85% (i.e., 85%, 86%, 87%, 88%, 89%, 90%, 91%, 92%, 93%, 94%, 95%, 96%, 97%, 98%, 99%, or 100%) identity to the amino acid sequence set forth in any one of SEQ ID NOs.: 22, 30, 32, 34, 35, 37, 45, 47, 49, 50, 52, 54, 62, 64, 66, 68, 69, 71, 81, 91, 101, 111, 121, 135, 145, 155, 158, 180, 190, 200, 210, 220, 232, 242, 252, 253, 255, 256, 258, 260, 262, 264, 266, 267, 270, 272, 274, 276, 278, 280, 284, 286, 288, 291, 292, 295, 297, 298, 300, 304, 314, 348, 358, 368, 378, 388, 398, 408, 418, 428, 432, 434, 437, 446, 448, 458, 459, and 460, and wherein the first VL and the second VL are different and each independently comprise an amino acid sequence having at least 85% (i.e., 85%, 86%, 87%, 88%, 89%, 90%, 91%, 92%, 93%, 94%, 95%, 96%, 97%, 98%, 99%, or 100%) identity to the amino acid sequence set forth in any one of SEQ ID NOs.: 26, 41, 58, 75, 85, 95, 105, 115, 125, 139, 149, 184, 194, 204, 214, 224, 230, 236, 246, 282, 302, 308, 319, 352, 362, 372, 382, 392, 402, 412, 422, 439, 442, 443, 444, and 445, and wherein the first VH and the first VL together form a first antigen-binding site, and wherein the second VH and the second VL together form a second antigen-binding site.

In certain embodiments, the antibody or antigen-binding fragment comprises a Fc polypeptide, or a fragment thereof. The “Fc” fragment or Fc polypeptide comprises the carboxy-terminal portions (i.e., the CH2 and CH3 domains of IgG) of both antibody H chains held together by disulfides. Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation. As discussed herein, modifications (e.g., amino acid substitutions) may be made to an Fc domain in order to modify (e.g., improve, reduce, or ablate) one or more functionality of an Fc-containing polypeptide (e.g., an antibody of the present disclosure). Such functions include, for example, Fc receptor (FcR) binding, antibody half-life modulation (e.g., by binding to FcRn), ADCC function, protein A binding, protein G binding, and complement binding. Amino acid modifications that modify (e.g., improve, reduce, or ablate) Fc functionalities include, for example, the T250Q/M428L, M252Y/S254T/T256E, H433K/N434F, M428L/N434S, E233P/L234V/L235A/G236+A327G/A330S/P331S, E333A, S239D/A330L/1332E, P2571/Q311, K326W/E333S, S239D/1332E/G236A, N297Q, K322A, S228P, L235E+E318A/K320A/K322A, L234A/L235A (also referred to herein as “LALA”), and L234A/L235A/P329G mutations, which mutations are summarized and annotated in “Engineered Fc Regions”, published by InvivoGen (2011) and available online at invivogen.com/PDF/review/review-Engineered-Fc-Regions-invivogen.pdf?utm_source=review&utm_medium=pdf&utm_campaign=review&utm_content=Engineered-Fc-Regions, and are incorporated herein by reference.

For example, to activate the complement cascade, the C1q protein complex can bind to at least two molecules of IgG1 or one molecule of IgM when the immunoglobulin molecule(s) is attached to the antigenic target (Ward, E. S., and Ghetie, V., Ther. Immunol. 2 (1995) 77-94). Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to C1q. The role of Glu318, Lys320 and Lys 322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on cells including hematopoietic cells. Fc receptors belong to the immunoglobulin superfamily, and shown to mediate both the removal of antibody-coated pathogens by phagocytosis of immune complexes, and the lysis of erythrocytes and various other cellular targets (e.g., tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcγR, for IgE as FcR, for IgA as FcαR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., J Lab. Clin. Med. 126 (1995) 330-341; and Gessner, J. E., et al., Ann. Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Fc moieties providing cross-linking of receptors (e.g., FcγR) are contemplated herein. In humans, three classes of FcγR have been characterized to-date, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is believed to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homologous; and (iii) FcγRIII (CD16), which binds IgG with medium to low affinity and has been found in two forms: FcγRIIIA, which has been found on NK cells, macrophages, eosinophils, and some monocytes and T cells, and is believed to mediate ADCC; and FcγRIIIB, which is highly expressed on neutrophils. FcγRIIA is found on many cells involved in killing (e.g., macrophages, monocytes, neutrophils) and seems able to activate the killing process. FcγRIIB seems to play a role in inhibitory processes and is found on B-cells, macrophages and on mast cells and eosinophils. Importantly, it has been shown that 75% of all FcγRIIB is found in the liver (Ganesan, L. P. et al., 2012: “FcγRIIb on liver sinusoidal endothelium clears small immune complexes,” Journal of Immunology 189: 4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189: 4981-4988).

In some embodiments, the antibodies disclosed herein and the antigen-binding fragments thereof comprise an Fc polypeptide or fragment thereof for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). In some embodiments, the antibodies of the present disclosure, or the antigen binding fragments thereof, comprise an engineered Fc moiety with the mutations S267E and L328F, in particular as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933.

On B cells, FcγRIIB may function to suppress further immunoglobulin production and isotype switching to, for example, the IgE class. On macrophages, FcγRIIB is thought to inhibit phagocytosis as mediated through FcγRIIA. On eosinophils and mast cells, the B form may help to suppress activation of these cells through IgE binding to its separate receptor.

Regarding FcγRI binding, modification in native IgG of at least one of E233-G236, P238, D265, N297, A327 and P329 reduces binding to FcγRI. IgG2 residues at positions 233-236, substituted into corresponding positions IgG1 and IgG4, reduces binding of IgG1 and IgG4 to FcγRI by 10³-fold and eliminated the human monocyte response to antibody-sensitized red blood cells (Armour, K. L., et al. Eur. J. Immunol. 29 (1999) 2613-2624).

Regarding FcγRII binding, reduced binding for FcγRIIA is found, e.g., for IgG mutation of at least one of E233-G236, P238, D265, N297, A327, P329, D270, Q295, A327, R292 and K414.

Two allelic forms of human FcγRIIA are the “H131” variant, which binds to IgG1 Fc with high affinity, and the “R131” variant, which binds to IgG1 Fc with low affinity. See, e.g., Bruhns et al., Blood 113:3716-3725 (2009).

Regarding FcγRIII binding, reduced binding to FcγRIIIA is found, e.g., for mutation of at least one of E233-G236, P238, D265, N297, A327, P329, D270, Q295, A327, 5239, E269, E293, Y296, V303, A327, K338 and D376. Mapping of the binding sites on human IgG1 for Fc receptors, the above-mentioned mutation sites, and methods for measuring binding to FcγRI and FcγRIIA, are described in Shields, R. L., et al., J. Biol. Chem. 276 (2001) 6591-6604.

Two allelic forms of human FcγRIIIA are the “F158” variant, which binds to IgG1 Fc with low affinity, and the “V158” variant, which binds to IgG1 Fc with high affinity. See, e.g., Bruhns et al., Blood 113:3716-3725 (2009).

Regarding binding to FcγRII, two regions of native IgG Fc appear to be involved in interactions between FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g., in a region of P331 (Wines, B. D., et al., J. Immunol. 2000; 164: 5313-5318). Moreover, FcγRI appears to bind to the same site on IgG Fc, whereas FcRn and Protein A bind to a different site on IgG Fe, which appears to be at the CH2-CH3 interface (Wines, B. D., et al., J. Immunol. 2000; 164: 5313-5318).

Also contemplated are mutations that increase binding affinity of an Fc polypeptide or fragment thereof of the present disclosure to a (i.e., one or more) Fcγ receptor (e.g., as compared to a reference Fc polypeptide or fragment thereof or containing the same that does not comprise the mutation(s)). See, e.g., Delillo and Ravetch, Cell 161(5):1035-1045 (2015) and Ahmed et al., J. Struc. Biol. 194(1):78 (2016), the Fc mutations and techniques of which are incorporated herein by reference.

In any of the herein disclosed embodiments, an antibody or antigen-binding fragment can comprise a Fc polypeptide or fragment thereof comprising a mutation selected from G236A; S239D; A330L; and 1332E; or a combination comprising any two or more of the same; e.g., S239D/I332E; S239D/A330L/I332E; G236A/S239D/I332E; G236A/A330L/I332E (also referred to herein as “GAALIE”); or G236A/S239D/A330L/I332E. In some embodiments, the Fc polypeptide or fragment thereof does not comprise S239D. In some embodiments, the Fc polypeptide or fragment thereof comprises S at position 239.

In certain embodiments, the Fc polypeptide or fragment thereof may comprise or consist of at least a portion of an Fc polypeptide or fragment thereof that is involved in binding to FcRn binding. In certain embodiments, the Fc polypeptide or fragment thereof comprises one or more amino acid modifications that improve binding affinity for (e.g., enhance binding to) FcRn (e.g., at a pH of about 6.0) and, in some embodiments, thereby extend in vivo half-life of a molecule comprising the Fc polypeptide or fragment thereof (e.g., as compared to a reference Fc polypeptide or fragment thereof or antibody that is otherwise the same but does not comprise the modification(s)). In certain embodiments, the Fc polypeptide or fragment thereof comprises or is derived from a IgG Fc and a half-life-extending mutation comprises any one or more of: M428L; N434S; N434H; N434A; N434S; M252Y; S254T; T256E; T250Q; P257I Q31 11; D376V; T307A; E380A (EU numbering). In certain embodiments, a half-life-extending mutation comprises M428L/N434S (also referred to herein as “MLNS” and “LS”). In certain embodiments, a half-life-extending mutation comprises M252Y/S254T/T256E. In certain embodiments, a half-life-extending mutation comprises T250Q/M428L. In certain embodiments, a half-life-extending mutation comprises P257I/Q311I. In certain embodiments, a half-life-extending mutation comprises P257I/N434H. In certain embodiments, a half-life-extending mutation comprises D376V/N434H. In certain embodiments, a half-life-extending mutation comprises T307A/E380A/N434A.

In some embodiments, an antibody or antigen-binding fragment includes a Fc moiety that comprises the substitution mutations M428L/N434S. In some embodiments, an antibody or antigen-binding fragment includes a Fc polypeptide or fragment thereof that comprises the substitution mutations G236A/A330L/I332E. In certain embodiments, an antibody or antigen-binding fragment includes a (e.g., IgG) Fc moiety that comprises a G236A mutation, an A330L mutation, and a 1332E mutation (GAALIE), and does not comprise a S239D mutation (e.g., comprises a native S at position 239). In particular embodiments, an antibody or antigen-binding fragment includes an Fc polypeptide or fragment thereof that comprises the substitution mutations: M428L/N434S and G236A/A330L/I332E, and optionally does not comprise S239D. In certain embodiments, an antibody or antigen-binding fragment includes a Fc polypeptide or fragment thereof that comprises the substitution mutations: M428L/N434S and G236A/S239D/A330L/I332E.

In certain embodiments, the antibody or antigen-binding fragment comprises a mutation that alters glycosylation, wherein the mutation that alters glycosylation comprises N297A, N297Q, or N297G, and/or the antibody or antigen-binding fragment is partially or fully aglycosylated and/or is partially or fully afucosylated. Host cell lines and methods of making partially or fully aglycosylated or partially or fully afucosylated antibodies and antigen-binding fragments are known (see, e.g., PCT Publication No. WO 2016/181357; Suzuki et al. Clin. Cancer Res. 13(6):1875-82 (2007); Huang et al. MAbs 6:1-12 (2018)).

In certain embodiments, the antibody or antigen-binding fragment is capable of eliciting continued protection in vivo in a subject even once no detectable levels of the antibody or antigen-binding fragment can be found in the subject (i.e., when the antibody or antigen-binding fragment has been cleared from the subject following administration). Such protection is referred to herein as a vaccinal effect. Without wishing to be bound by theory, it is believed that dendritic cells can internalize complexes of antibody and antigen and thereafter induce or contribute to an endogenous immune response against antigen. In certain embodiments, an antibody or antigen-binding fragment comprises one or more modifications, such as, for example, mutations in the Fc comprising G236A, A330L, and 1332E, that are capable of activating dendritic cells that may induce, e.g., T cell immunity to the antigen.

In any of the presently disclosed embodiments, the antibody or antigen-binding fragment comprises a Fc polypeptide or a fragment thereof, including a CH2 (or a fragment thereof, a CH3 (or a fragment thereof), or a CH2 and a CH3, wherein the CH2, the CH3, or both can be of any isotype and may contain amino acid substitutions or other modifications as compared to a corresponding wild-type CH2 or CH3, respectively. In certain embodiments, a Fc polypeptide of the present disclosure comprises two CH2-CH3 polypeptides that associate to form a dimer.

In any of the presently disclosed embodiments, the antibody or antigen-binding fragment can be monoclonal. The term “monoclonal antibody” (mAb) as used herein refers to an antibody obtained from a population of substantially homogeneous antibodies, i.e., individual antibodies comprising the population are identical except for possible naturally occurring mutations that may be present, in some cases in minor amounts. Monoclonal antibodies are highly specific, being directed against a single antigenic site. Furthermore, in contrast to polyclonal antibody preparations that include different antibodies directed against different epitopes, each monoclonal antibody is directed against a single epitope of the antigen. In addition to their specificity, the monoclonal antibodies are advantageous in that they may be synthesized uncontaminated by other antibodies. The term “monoclonal” is not to be construed as requiring production of the antibody by any particular method. For example, monoclonal antibodies useful in the present invention may be prepared by the hybridoma methodology first described by Kohler et al., Nature 256:495 (1975), or may be made using recombinant DNA methods in bacterial, eukaryotic animal, or plant cells (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:581-597 (1991), for example. Monoclonal antibodies may also be obtained using methods disclosed in PCT Publication No. WO 2004/076677A2.

Antibodies and antigen-binding fragments of the present disclosure include “chimeric antibodies” in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, U.S. Pat. Nos. 4,816,567; 5,530,101 and 7,498,415; and Morrison et al., Proc. Natl. Acad. Sci. USA, 81:6851-6855 (1984)). For example, chimeric antibodies may comprise human and non-human residues. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). Chimeric antibodies also include primatized and humanized antibodies.

A “humanized antibody” is generally considered to be a human antibody that has one or more amino acid residues introduced into it from a source that is non-human. These non-human amino acid residues are typically taken from a variable domain. Humanization may be performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting non-human variable sequences for the corresponding sequences of a human antibody. Accordingly, such “humanized” antibodies are chimeric antibodies (U.S. Pat. Nos. 4,816,567; 5,530,101 and 7,498,415) wherein substantially less than an intact human variable domain has been substituted by the corresponding sequence from a non-human species. In some instances, a “humanized” antibody is one which is produced by a non-human cell or animal and comprises human sequences, e.g., Hc domains.

A “human antibody” is an antibody containing only sequences that are present in an antibody that is produced by a human. However, as used herein, human antibodies may comprise residues or modifications not found in a naturally occurring human antibody (e.g., an antibody that is isolated from a human), including those modifications and variant sequences described herein. These are typically made to further refine or enhance antibody performance. In some instances, human antibodies are produced by transgenic animals. For example, see U.S. Pat. Nos. 5,770,429; 6,596,541 and 7,049,426.

In certain embodiments, an antibody or antigen-binding fragment of the present disclosure is chimeric, humanized, or human.

### Polynucleotides, Vectors, and Host Cells

In another aspect, the present disclosure provides isolated polynucleotides that encode any of the presently disclosed antibodies or an antigen-binding fragment thereof, or a portion thereof (e.g., a CDR, a VH, a VL, a heavy chain, or a light chain). In certain embodiments, the polynucleotide is codon-optimized for expression in a host cell. Once a coding sequence is known or identified, codon optimization can be performed using known techniques and tools, e.g., using the GenScript® OptimiumGene™ tool; see also Scholten et al., Clin. Immunol. 119:135, 2006). Codon-optimized sequences include sequences that are partially codon-optimized (i.e., one or more codon is optimized for expression in the host cell) and those that are fully codon-optimized.

It will also be appreciated that polynucleotides encoding antibodies and antigen-binding fragments of the present disclosure may possess different nucleotide sequences while still encoding a same antibody or antigen-binding fragment due to, for example, the degeneracy of the genetic code, splicing, and the like.

In certain embodiments, the polynucleotide comprises a polynucleotide having at least 50% (i.e., 50%, 55%, 60%, 65%, 70%, 75%, 80%, 85%, 90%, 91%, 92%, 93%, 94%, 95%, 96%, 97%, 98%, 99%, or 100%) identity to the polynucleotide sequence according to any one or more of SEQ ID NOs.:79, 80, 89, 90, 99, 100, 109, 110, 119, 120, 129-134, 143, 144, 153, 154, 157, 159, 188, 189, 198, 199, 208, 209, 218, 219, 228, 229, 231, 240, 241, 250, 251, 312, 313, 322, 323, 356, 357, 366, 367, 376, 377, 386, 387, 396, 397, 406, 407, 416, 417, 426, 427, 429, 430, 431, 433, 436, 438, and 441.

It will be appreciated that in certain embodiments, a polynucleotide encoding an antibody or antigen-binding fragment is comprised in a polynucleotide that includes other sequences and/or features for, e.g., expression of the antibody or antigen-binding fragment in a host cell. Exemplary features include a promoter sequence, a polyadenylation sequence, a sequence that encodes a signal peptide (e.g., located at the N-terminus of a expressed antibody heavy chain or light chain), or the like. Accordingly, in some embodiments, a polynucleotide further comprises a polynucleotide sequence having at least 50% identity to, comprising, or consisting of the polynucleotide sequence set forth in any one of SEQ ID NOs.:451-453 and 455. In some embodiments, a polynucleotide comprises a sequence that encodes a signal peptide (also referred-to as a leader sequence) having at least 90% to, comprising, or consisting of the amino acid sequence set forth in SEQ ID NO.: 454 or SEQ ID NO.: 456.

In any of the presently disclosed embodiments, the polynucleotide can comprise deoxyribonucleic acid (DNA) or ribonucleic acid (RNA). In some embodiments, the RNA comprises messenger RNA (mRNA).

Vectors are also provided, wherein the vectors comprise or contain a polynucleotide as disclosed herein (e.g., a polynucleotide that encodes an antibody or antigen-binding fragment that binds to SARS-CoV-2). A vector can comprise any one or more of the vectors disclosed herein. In particular embodiments, a vector is provided that comprises a DNA plasmid construct encoding the antibody or antigen-binding fragment, or a portion thereof (e.g., so-called “DMAb”; see, e.g., Muthumani et al., J Infect Dis. 214(3):369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). In certain embodiments, a DNA plasmid construct comprises a single open reading frame encoding a heavy chain and a light chain (or a VH and a VL) of the antibody or antigen-binding fragment, wherein the sequence encoding the heavy chain and the sequence encoding the light chain are optionally separated by polynucleotide encoding a protease cleavage site and/or by a polynucleotide encoding a self-cleaving peptide. In some embodiments, the substituent components of the antibody or antigen-binding fragment are encoded by a polynucleotide comprised in a single plasmid. In other embodiments, the substituent components of the antibody or antigen-binding fragment are encoded by a polynucleotide comprised in two or more plasmids (e.g., a first plasmid comprises a polynucleotide encoding a heavy chain, VH, or VH+CH, and a second plasmid comprises a polynucleotide encoding the cognate light chain, VL, or VL+CL). In certain embodiments, a single plasmid comprises a polynucleotide encoding a heavy chain and/or a light chain from two or more antibodies or antigen-binding fragments of the present disclosure. An exemplary expression vector is pVax1, available from Invitrogen®. A DNA plasmid of the present disclosure can be delivered to a subject by, for example, electroporation (e.g., intramuscular electroporation), or with an appropriate formulation (e.g., hyaluronidase).

An exemplary expression vector is pVax1, available from Invitrogen®. A DNA plasmid of the present disclosure can be delivered to a subject by, for example, electroporation (e.g., intramuscular electroporation), or with an appropriate formulation (e.g., hyaluronidase). In some embodiments, a vector of the present disclosure comprises a nucleotide sequence encoding a signal peptide. The signal peptide may or may not be present (e.g., can be enzymatically cleaved from) on the mature antibody or antigen-binding fragment. In certain embodiments, the signal peptide is encoded by a nucleotide sequence as set forth in SEQ ID NO.: 452 or SEQ ID NO.: 455, and/or the signal peptide comprises or consists of the amino acid sequence set forth in SEQID NO.:454 or SEQ ID NO.: 456. In some embodiments, a vector of the present disclosure comprises a polyadenylation signal sequence. In certain embodiments, the polyadenylation signal sequence comprises or consists of the nucleotide sequence as set forth in SEQ ID NO.: 453.

In some embodiments, a vector of the present disclosure comprises a CMV promoter. In certain embodiments, the promoter comprises or consists of the nucleotide sequence as set forth in SEQ ID NO.: 451.

In a further aspect, the present disclosure also provides a host cell expressing an antibody or antigen-binding fragment according to the present disclosure; or comprising or containing a vector or polynucleotide according the present disclosure.

Examples of such cells include but are not limited to, eukaryotic cells, e.g., yeast cells, animal cells, insect cells, plant cells; and prokaryotic cells, including E. coli. In some embodiments, the cells are mammalian cells. In certain such embodiments, the cells are a mammalian cell line such as CHO cells (e.g., DHFR-CHO cells (Urlaub et al., PNAS 77:4216 (1980)), human embryonic kidney cells (e.g., HEK293T cells), PER.C6 cells, Y0 cells, Sp2/0 cells. NS0 cells, human liver cells, e.g., Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

In particular embodiments, the cell may be transfected with a vector according to the present description with an expression vector. The term “transfection” refers to the introduction of nucleic acid molecules, such as DNA or RNA (e.g., mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine, etc. In certain embodiments, the introduction is non-viral.

Moreover, host cells of the present disclosure may be transfected stably or transiently with a vector according to the present disclosure, e.g., for expressing an antibody, or an antigen-binding fragment thereof, according to the present disclosure. In such embodiments, the cells may be stably transfected with the vector as described herein. Alternatively, cells may be transiently transfected with a vector according to the present disclosure encoding an antibody or antigen-binding fragment as disclosed herein. In any of the presently disclosed embodiments, a polynucleotide may be heterologous to the host cell.

Accordingly, the present disclosure also provides recombinant host cells that heterologously express an antibody or antigen-binding fragment of the present disclosure. For example, the cell may be of a species that is different to the species from which the antibody was fully or partially obtained (e.g., CHO cells expressing a human antibody or an engineered human antibody). In some embodiments, the cell type of the host cell does not express the antibody or antigen-binding fragment in nature. Moreover, the host cell may impart a post-translational modification (PTM; e.g., glysocylation or fucosylation) on the antibody or antigen-binding fragment that is not present in a native state of the antibody or antigen-binding fragment (or in a native state of a parent antibody from which the antibody or antigen binding fragment was engineered or derived). Such a PTM may result in a functional difference (e.g., reduced immunogenicity). Accordingly, an antibody or antigen-binding fragment of the present disclosure that is produced by a host cell as disclosed herein may include one or more post-translational modification that is distinct from the antibody (or parent antibody) in its native state (e.g., a human antibody produced by a CHO cell can comprise a more post-translational modification that is distinct from the antibody when isolated from the human and/or produced by the native human B cell or plasma cell).

Insect cells useful expressing a binding protein of the present disclosure are known in the art and include, for example, Spodoptera frugipera Sf9 cells, Trichoplusia ni BTI-TN5B1-4 cells, and Spodoptera frugipera SfSWT01 “Mimic™” cells. See, e.g., Palmberger et al., J. Biotechnol. 153(3-4):160-166 (2011). Numerous baculoviral strains have been identified which may be used in conjunction with insect cells, particularly for transfection of Spodoptera frugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210-215 (2006).

Plant cells can also be utilized as hosts for expressing a binding protein of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

In certain embodiments, the host cell comprises a mammalian cell. In particular embodiments, the host cell is a CHO cell, a HEK293 cell, a PER.C6 cell, a Y0 cell, a Sp2/0 cell, a NS0 cell, a human liver cell, a myeloma cell, or a hybridoma cell.

In a related aspect, the present disclosure provides methods for producing an antibody, or antigen-binding fragment, wherein the methods comprise culturing a host cell of the present disclosure under conditions and for a time sufficient to produce the antibody, or the antigen-binding fragment. Methods useful for isolating and purifying recombinantly produced antibodies, by way of example, may include obtaining supernatants from suitable host cell/vector systems that secrete the recombinant antibody into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant antibody described herein include batch cell culture, which is monitored and controlled to maintain appropriate culture conditions. Purification of soluble antibodies may be performed according to methods described herein and known in the art and that comport with laws and guidelines of domestic and foreign regulatory agencies.

Compositions

Also provided herein are compositions that comprise any one or more of the presently disclosed antibodies, antigen-binding fragments, polynucleotides, vectors, or host cells, singly or in any combination, and can further comprise a pharmaceutically acceptable carrier, excipient, or diluent. Carriers, excipients, and diluents are discussed in further detail herein.

In certain embodiments, a composition comprises a plurality of an antibody and/or an antigen-binding fragment of the present disclosure, wherein one or more antibody or antigen-binding fragment does not comprise a lysine residue at the C-terminal end of the heavy chain, CH1-CH3, or Fc polypeptide, and wherein one or more antibody or antigen-binding fragment comprises a lysine residue at the C-terminal end of the heavy chain, CH1-CH3, or Fc polypeptide.

In certain embodiments, a composition comprises two or more different antibodies or antigen-binding fragments according to the present disclosure. In certain embodiments, antibodies or antigen-binding fragments to be used in a combination each independently have one or more of the following characteristics: neutralize naturally occurring SARS-CoV-2 variants; do not compete with one another for Spike protein binding; bind distinct Spike protein epitopes; have a reduced formation of resistance to SARS-CoV-2; when in a combination, have a reduced formation of resistance to SARS-CoV-2; potently neutralize live SARS-CoV-2 virus; exhibit additive or synergistic effects on neutralization of live SARS-CoV-2 virus when used in combination; exhibit effector functions; are protective in relevant animal model(s) of infection; are capable of being produced in sufficient quantities for large-scale production.

In certain embodiments, antibodies or antigen-binding fragments to be used in a combination each independently have one or more of the following characteristics: neutralize one, two, three, four, five, or more naturally occurring sarbecovirus variants; do not compete with one another for Spike protein binding; bind distinct sarbecovirus Spike protein epitopes; have a reduced formation of resistance to sarbecovirus; when in a combination, have a reduced formation of resistance to sarbecovirus; potently neutralize one, two, three, four, five or more live sarbecoviruses; exhibit additive or synergistic effects on neutralization of one, two, three, four, five or more or more live sarbecoviruses when used in combination; exhibit effector functions; are protective in relevant animal model(s) of infection; are capable of being produced in sufficient quantities for large-scale production.

In certain embodiments, a composition comprises two or more different antibodies or antigen-binding fragments according to the present disclosure. In certain embodiments, a composition comprises a first antibody or antigen-binding fragment, comprising a VH comprising or consisting of the amino acid sequence as set forth in SEQ ID NO: 172 and a VL comprising or consisting of the amino acid sequence as set forth in SEQ ID NO: 176; and a second antibody or antigen-binding fragment comprising a VH comprising or consisting of the amino acid sequence as set forth in any one of SEQ ID NOs: 22, 30, 32, 34, 35, 37, 45, 47, 49, 50, 52, 54, 62, 64, 66, 68, or 69, and a VL comprising of consisting of the amino acid sequence as set forth in any one of SEQ ID NOs: 26, 41, or 58. In certain embodiments, a composition comprises a first antibody or antigen-binding fragment comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 173-175, respectively, and the CDRL1, CDRL2, and CDRL3 comprise or consist of the amino acid sequences set forth in SEQ ID NOs: 177-179, respectively, and a second antibody or antigen-binding fragment comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in (i) SEQ ID NOs: 23-25, respectively, (ii) SEQ ID NOs: 160-162, respectively, (iii) SEQ ID NOs: 38-40, respectively, or (iv) SEQ ID NOs: 166-168, respectively, and the CDRL1, CDRL2, and CDRL3 comprise or consist of the amino acid sequences set forth in (i) SEQ ID NOs: 27-29, respectively, (ii) SEQ ID NOs: 163-165, respectively, (iii) SEQ ID NOs: 42-44, respectively, or (iv) SEQ ID NOs: 169-171, respectively.

In certain embodiments, a composition comprises a first antibody or antigen-binding fragment comprising a VH comprising or consisting of the amino acid sequence as set forth in SEQ ID NO.: 172 and a VL comprising or consisting of the amino acid sequence as set forth in SEQ ID NO.: 176; and a second antibody or antigen-binding fragment comprising a VH comprising or consisting of the amino acid sequence as set forth in SEQ ID NO.: 200 and a VL comprising or consisting of the amino acid sequence as set forth in SEQ ID NO.: 204. In certain embodiments, a composition comprises a first antibody or antigen-binding fragment comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 173-175, respectively, and the CDRL1, CDRL2, and CDRL3 comprise or consist of the amino acid sequences set forth in SEQ ID NOs: 177-179, respectively, and a second antibody or antigen-binding fragment comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 201-203, respectively, and the CDRL1, CDRL2, and CDRL3 comprise or consist of the amino acid sequences set forth in SEQ ID NOs: 205-207, respectively.

In certain embodiments, a composition comprises a first vector comprising a first plasmid, and a second vector comprising a second plasmid, wherein the first plasmid comprises a polynucleotide encoding a heavy chain, VH, or VH+CH, and a second plasmid comprises a polynucleotide encoding the cognate light chain, VL, or VL+CL of the antibody or antigen-binding fragment thereof. In certain embodiments, a composition comprises a polynucleotide (e.g., mRNA) coupled to a suitable delivery vehicle or carrier. Exemplary vehicles or carriers for administration to a human subject include a lipid or lipid-derived delivery vehicle, such as a liposome, solid lipid nanoparticle, oily suspension, submicron lipid emulsion, lipid microbubble, inverse lipid micelle, cochlear liposome, lipid microtubule, lipid microcylinder, or lipid nanoparticle (LNP) or a nanoscale platform (see, e.g., Li et al. Wilery Interdiscip Rev. Nanomed Nanobiotechnol. 11(2):e1530 (2019)). Principles, reagents, and techniques for designing appropriate mRNA and formulating mRNA-LNP and delivering the same are described in, for example, Pardi et al. (J Control Release 217345-351 (2015)); Thess et al. (Mol Ther 23: 1456-1464 (2015)); Thran et al. (EMBO Mol Med 9(10):1434-1448 (2017); Kose et al. (Sci. Immunol. 4 eaaw6647 (2019); and Sabnis et al. (Mol. Ther. 26:1509-1519 (2018)), which techniques, include capping, codon optimization, nucleoside modification, purification of mRNA, incorporation of the mRNA into stable lipid nanoparticles (e.g., ionizable cationic lipid/phosphatidylcholine/cholesterol/PEG-lipid; ionizable lipid:distearoyl PC:cholesterol: polyethylene glycol lipid), and subcutaneous, intramuscular, intradermal, intravenous, intraperitoneal, and intratracheal administration of the same, are incorporated herein by reference.

### Methods and Uses

Also provided herein are methods for use of an antibody or antigen-binding fragment, nucleic acid, vector, cell, or composition of the present disclosure in the diagnosis of a sarbecovirus infection, such as a SARS-CoV-2 infection (e.g., in a human subject, or in a sample obtained from a human subject).

Methods of diagnosis (e.g., in vitro, ex vivo) may include contacting an antibody, antibody fragment (e.g., antigen binding fragment) with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood. The methods of diagnosis may also include the detection of an antigen/antibody complex, in particular following the contacting of an antibody or antibody fragment with a sample. Such a detection step can be performed at the bench, i.e., without any contact to the human or animal body. Examples of detection methods are well-known to the person skilled in the art and include, e.g., ELISA (enzyme-linked immunosorbent assay), including direct, indirect, and sandwich ELISA.

Also provided herein are methods of treating a subject using an antibody or antigen-binding fragment of the present disclosure, or a composition comprising the same, wherein the subject has, is believed to have, or is at risk for having an infection by a sarbecorvirus, such as SARS-CoV-2. “Treat,” “treatment,” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising an antibody or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof. In certain embodiments, therapeutic or prophylactic/preventive benefit includes reduction or prevention of hospitalization for treatment of a sarbecovirus infection, such as a SARS-CoV-2 infection (i.e., in a statistically significant manner). In certain embodiments, therapeutic or prophylactic/preventive benefit includes a reduced duration of hospitalization for treatment of a sarbecovirus infection, such as a SARS-CoV-2 infection (i.e., in a statistically significant manner). In certain embodiments, therapeutic or prophylactic/preventive benefit includes a reduced or abrogated need for respiratory intervention, such as intubation and/or the use of a respirator device. In certain embodiments, therapeutic or prophylactic/preventive benefit includes reversing a late-stage disease pathology and/or reducing mortality.

A “therapeutically effective amount” or “effective amount” of an antibody, antigen-binding fragment, polynucleotide, vector, host cell, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously. A combination may comprise, for example, two different antibodies that specifically bind a SARS-CoV-2 antigen, which in certain embodiments, may be the same or different SARS-CoV-2 antigen, and/or can comprise the same or different epitopes.

Accordingly, in certain embodiments, methods are provided for treating a sarbecovirus infection, such as a SARS-CoV-2 infection, in a subject, wherein the methods comprise administering to the subject an effective amount of an antibody, antigen-binding fragment, polynucleotide, vector, host cell, or composition as disclosed herein.

Subjects that can be treated by the present disclosure are, in general, human and other primate subjects, such as monkeys and apes for veterinary medicine purposes. Other model organisms, such as mice and rats, may also be treated according to the present disclosure. In any of the aforementioned embodiments, the subject may be a human subject. The subjects can be male or female and can be any suitable age, including infant, juvenile, adolescent, adult, and geriatric subjects.

A number of criteria are believed to contribute to high risk for severe symptoms or death associated with a SARS CoV-2 infection. These include, but are not limited to, age, occupation, general health, pre-existing health conditions, and lifestyle habits. In some embodiments, a subject treated according to the present disclosure comprises one or more risk factors.

In certain embodiments, a human subject treated according to the present disclosure is an infant, a child, a young adult, an adult of middle age, or an elderly person. In certain embodiments, a human subject treated according to the present disclosure is less than 1 year old, or is 1 to 5 years old, or is between 5 and 125 years old (e.g., 5, 10, 15, 20, 25, 30, 35, 40, 45, 50, 55, 60, 65, 70, 75, 80, 85, 90, 95, 100, 105, 110, 115, or 125 years old, including any and all ages therein or therebetween). In certain embodiments, a human subject treated according to the present disclosure is 0-19 years old, 20-44 years old, 45-54 years old, 55-64 years old, 65-74 years old, 75-84 years old, or 85 years old, or older. Persons of middle, and especially of elderly age are believed to be at particular risk. In particular embodiments, the human subject is 45-54 years old, 55-64 years old, 65-74 years old, 75-84 years old, or 85 years old, or older. In some embodiments, the human subject is male. In some embodiments, the human subject is female.

In certain embodiments, a human subject treated according to the present disclosure is a resident of a nursing home or a long-term care facility, is a hospice care worker, is a healthcare provider or healthcare worker, is a first responder, is a family member or other close contact of a subject diagnosed with or suspected of having a SARS-CoV-2 infection, is overweight or clinically obese, is or has been a smoker, has or had chronic obstructive pulmonary disease (COPD), is asthmatic (e.g., having moderate to severe asthma), has an autoimmune disease or condition (e.g., diabetes), and/or has a compromised or depleted immune system (e.g., due to AIDS/HIV infection, a cancer such as a blood cancer, a lymphodepleting therapy such as a chemotherapy, a bone marrow or organ transplantation, or a genetic immune condition), has chronic liver disease, has cardiovascular disease, has a pulmonary or heart defect, works or otherwise spends time in close proximity with others, such as in a factory, shipping center, hospital setting, or the like.

In certain embodiments, a subject treated according to the present disclosure has received a vaccine for SARS-CoV-2 and the vaccine is determined to be ineffective, e.g., by post-vaccine infection or symptoms in the subject, by clinical diagnosis or scientific or regulatory criteria.

In certain embodiments, treatment is administered as peri-exposure prophylaxis. In certain embodiments, treatment is administered to a subject with mild-to-moderate disease, which may be in an outpatient setting. In certain embodiments, treatment is administered to a subject with moderate-to-severe disease, such as requiring hospitalization.

Typical routes of administering the presently disclosed compositions thus include, without limitation, oral, topical, transdermal, inhalation, parenteral, sublingual, buccal, rectal, vaginal, and intranasal. The term “parenteral”, as used herein, includes subcutaneous injections, intravenous, intramuscular, intrasternal injection or infusion techniques. In certain embodiments, administering comprises administering by a route that is selected from oral, intravenous, parenteral, intragastric, intrapleural, intrapulmonary, intrarectal, intradermal, intraperitoneal, intratumoral, subcutaneous, topical, transdermal, intracisternal, intrathecal, intranasal, and intramuscular. In particular embodiments, a method comprises orally administering the antibody, antigen-binding fragment, polynucleotide, vector, host cell, or composition to the subject.

Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described an antibody or antigen-binding in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of an antibody or antigen-binding fragment, polynucleotide, vector, host cell, , or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi solid, semi liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of an antibody or antigen-binding fragment as herein disclosed such that a suitable dosage will be obtained. Typically, this amount is at least 0.01% of the antibody or antigen-binding fragment in the composition. When intended for oral administration, this amount may be varied to be between 0.1 and about 70% of the weight of the composition. Certain oral pharmaceutical compositions contain between about 4% and about 75% of the antibody or antigen-binding fragment. In certain embodiments, pharmaceutical compositions and preparations according to the present invention are prepared so that a parenteral dosage unit contains between 0.01 to 10% by weight of antibody or antigen-binding fragment prior to dilution.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the antibody or antigen-binding fragment of the disclosure and thereby assists in the delivery of the compound. Suitable agents that may act in this capacity include monoclonal or polyclonal antibodies, one or more proteins or a liposome. The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi phasic, or tri phasic systems in order to deliver the active ingredient(s). Delivery of the aerosol includes the necessary container, activators, valves, subcontainers, and the like, which together may form a kit. One of ordinary skill in the art, without undue experimentation, may determine preferred aerosols.

It will be understood that compositions of the present disclosure also encompass carrier molecules for polynucleotides, as described herein (e.g., lipid nanoparticles, nanoscale delivery platforms, and the like).

The pharmaceutical compositions may be prepared by methodology well known in the pharmaceutical art. For example, a composition intended to be administered by injection can be prepared by combining a composition that comprises an antibody, antigen-binding fragment thereof, or antibody conjugate as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the peptide composition so as to facilitate dissolution or homogeneous suspension of the antibody or antigen-binding fragment thereof in the aqueous delivery system.

In general, an appropriate dose and treatment regimen provide the composition(s) in an amount sufficient to provide therapeutic and/or prophylactic benefit (such as described herein, including an improved clinical outcome (e.g., a decrease in frequency, duration, or severity of diarrhea or associated dehydration, or inflammation, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions are administered in an effective amount (e.g., to treat a SARS-CoV-2 infection), which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In certain embodiments, following administration of therapies according to the formulations and methods of this disclosure, test subjects will exhibit about a 10% up to about a 99% reduction in one or more symptoms associated with the disease or disorder being treated as compared to placebo-treated or other suitable control subjects.

Generally, a therapeutically effective daily dose of an antibody or antigen binding fragment is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g). For polynucleotides, vectors, host cells, and related compositions of the present disclosure, a therapeutically effective dose may be different than for an antibody or antigen-binding fragment.

In certain embodiments, a method comprises administering the antibody, antigen-binding fragment, polynucleotide, vector, host cell, or composition to the subject at 2, 3, 4, 5, 6, 7, 8, 9, 10 times, or more.

In certain embodiments, a method comprises administering the antibody, antigen-binding fragment, or composition to the subject a plurality of times, wherein a second or successive administration is performed at about 6, about 7, about 8, about 9, about 10, about 11, about 12, about 24, about 48, about 74, about 96 hours, or more, following a first or prior administration, respectively.

In certain embodiments, a method comprises administering the antibody, antigen-binding fragment, polynucleotide, vector, host cell, or composition at least one time prior to the subject being infected by a sarbecovirus, such as SARS-CoV-2.

Compositions comprising an antibody, antigen-binding fragment, polynucleotide, vector, host cell, or composition of the present disclosure may also be administered simultaneously with, prior to, or after administration of one or more other therapeutic agents. Such combination therapy may include administration of a single pharmaceutical dosage formulation which contains a compound of the invention and one or more additional active agents, as well as administration of compositions comprising an antibody or antigen-binding fragment of the disclosure and each active agent in its own separate dosage formulation. For example, an antibody or antigen-binding fragment thereof as described herein and the other active agent can be administered to the patient together in a single oral dosage composition such as a tablet or capsule, or each agent administered in separate oral dosage formulations. Similarly, an antibody or antigen-binding fragment as described herein and the other active agent can be administered to the subject together in a single parenteral dosage composition such as in a saline solution or other physiologically acceptable solution, or each agent administered in separate parenteral dosage formulations. Where separate dosage formulations are used, the compositions comprising an antibody or antigen-binding fragment and one or more additional active agents can be administered at essentially the same time, i.e., concurrently, or at separately staggered times, i.e., sequentially and in any order; combination therapy is understood to include all these regimens.

In certain embodiments, a combination therapy is provided that comprises one or more anti-sarbecovirus antibody, such as an anti-SARS-CoV-2 antibody, (or one or more nucleic acid, host cell, vector, or composition) of the present disclosure and one or more anti-inflammatory agent and/or one or more anti-viral agent. In particular embodiments, the one or more anti-inflammatory agent comprises a corticosteroid such as, for example, dexamethasone, prednisone, or the like. In some embodiments, the one or more anti-inflammatory agents comprise a cytokine antagonist such as, for example, an antibody that binds to IL6 (such as siltuximab), or to IL-6R (such as tocilizumab), or to IL-1β, IL-7, IL-8, IL-9, IL-10, FGF, G-CSF, GM-CSF, IFN-7, IP-10, MCP-1, MIP-1A, MIP1-B, PDGR, TNF-α, or VEGF. In some embodiments, anti-inflammatory agents such as leronlimab, ruxolitinib and/or anakinra are used. In some embodiments, the one or more anti-viral agents comprise nucleotide analogs or nucleotide analog prodrugs such as, for example, remdesivir, sofosbuvir, acyclovir, and zidovudine. In particular embodiments, an anti-viral agent comprises lopinavir, ritonavir, favipiravir, or any combination thereof. Other anti-inflammatory agents for use in a combination therapy of the present disclosure include non-steroidal anti-inflammatory drugs (NSAIDS). It will be appreciated that in such a combination therapy, the one or more antibody (or one or more nucleic acid, host cell, vector, or composition) and the one or more anti-inflammatory agent and/or one or the more antiviral agent can be administered in any order and any sequence, or together.

In some embodiments, an antibody (or one or more nucleic acid, host cell, vector, or composition) is administered to a subject who has previously received one or more anti-inflammatory agent and/or one or more antiviral agent. In some embodiments, one or more anti-inflammatory agent and/or one or more antiviral agent is administered to a subject who has previously received an antibody (or one or more nucleic acid, host cell, vector, or composition).

In certain embodiments, a combination therapy is provided that comprises two or more anti-sarbecovirus antibodies of the present disclosure, such as two or more anti-SARS-CoV-2 antibodies. A method can comprise administering a first antibody to a subject who has received a second antibody, or can comprise administering two or more antibodies together. For example, in particular embodiments, a method is provided that comprises administering to the subject (a) a first antibody or antigen-binding fragment, when the subject has received a second antibody or antigen-binding fragment; (b) the second antibody or antigen-binding fragment, when the subject has received the first antibody or antigen-binding fragment; or (c) the first antibody or antigen-binding fragment, and the second antibody or antigen-binding fragment.

In a related aspect, uses of the presently disclosed antibodies, antigen-binding fragments, vectors, host cells, and compositions are provided.

In certain embodiments, an antibody, antigen-binding fragment, polynucleotide, vector, host cell, or composition is provided for use in a method of treating a SARS-CoV-2 infection in a subject.

In certain embodiments, an antibody, antigen-binding fragment, or composition is provided for use in a method of manufacturing or preparing a medicament for treating a sarbecovirus infection, such as a SARS-CoV-2 infection, in a subject.

TABLE 3
Sequences

| Sequence | SEQ ID | |
|---|---|---|
| Description | NO. | Sequence |
| SARS-CoV-2 | 1 | 1 attaaaggtt tataccttcc caggtaacaa accaaccaac |
| Wuhan | | tttcgatctc ttgtagatct 61 gttctctaaa cgaactttaa |
| seafood | | aatctgtgtg gctgtcactc ggctgcatgc ttagtgcact 121 |
| market | | cacgcagtat aattaataac taattactgt cgttgacagg |
| pneumonia | | acacgagtaa ctegtctatc 181 ttctgcaggc tgcttacggt |
| virus isolate | | ttcgtccgtg ttgcagccga tcatcagcac atctaggttt 241 |
| Wuhan-Hu-1 | | cgtccgggtg tgaccgaaag gtaagatgga gagccttgtc |
| genomic | | cctggtttca acgagaaaac 301 acacgtccaa ctcagtttgc |
| sequence | | ctgttttaca ggttcgcgac gtgctcgtac gtggctttgg 361 |
| (GenBank: | | agactccgtg gaggaggtct tatcagaggc cgtcaacat |
| MN908947.3; | | cttaaagatg gcacttgtgg 421 cttagtagaa gttgaaaaag |
| Jan. 23, 2020) | | gcgttttgcc tcaacttgaa cagccctatg tgttcatcaa 481 |
| | | acgttoggat gctcgaactg cacctcatgg tcatgttatg |
| | | gttgagctgg tagcagaact 541 cgaaggcatt cagtacggtc |
| | | gtagtggtga gacacttggt gtccttgtcc ctcatgtggg 601 |
| | | cgaaatacca gtggcttacc gcaaggttct tcttcgtaag |
| | | aacggtaata aaggagctgg 661 tggccatagt tacggcgccg |
| | | atctaaagtc atttgactta ggcgacgagc ttggcactga 721 |
| | | tccttatgaa gattttcaag aaaactggaa cactaaacat |
| | | agcagtggtg ttacccgtga 781 actcatgcgt gagcttaacg |
| | | gaggggcata cactcgctat gtcgataaca acttctgtgg 841 |
| | | ccctgatggc taccctcttg agtgcattaa agaccttcta |
| | | gcacgtgctg gtaaagcttc 901 atgcactttg tccgaacaac |
| | | tggactttat tgacactaag aggggtgtat actgctgccg 961 |
| | | tgaacatgag catgaaattg cttggtacac ggaacgttct |
| | | gaaaagagct atgaattgca 1021 gacacctttt gaaattaaat |
| | | tggcaaagaa atttgacacc ttcaatgggg aatgtccaaa 1081 |
| | | ttttgtattt cccttaaatt ccataatcaa gactattcaa ccaagggttg |
| | | aaaagaaaaa 1141 gcttgatggc tttatgggta gaattcgatc |
| | | tgtctatcca gttgcgtcac caaatgaatg 1201 caaccaaatg |
| | | tgcctttcaa ctctcatgaa gtgtgatcat tgtggtgaaa |
| | | cttcatggca 1261 gacgggcgat tttgttaaag ccacttgcga |
| | | attttgtggc actgagaatt tgactaaaga 1321 aggtgccact |
| | | acttgtggtt acttacccca aaatgctgtt gttaaaattt attgtccagc |
| | | 1381 atgtcacaat tcagaagtag gacctgagca tagtcttgcc |
| | | gaataccata atgaatctgg 1441 cttgaaaacc attcttgta |
| | | agggtggtcg cactattgcc tttggaggct gtgtgttctc |
| | | 1501 ttatgttggt tgccataaca agtgtgccta ttgggttcca |
| | | cgtgctagcg ctaacatagg 1561 ttgtaaccat acaggtgttg |
| | | ttggagaagg ttccgaaggt cttaatgaca accttcttga 1621 |
| | | aatactccaa aaagagaaag tcaacatcaa tattgttggt |
| | | gactttaaac ttaatgaaga 1681 gatcgccatt attttggcat |
| | | ctttttctgc ttccacaagt gcttttgtgg aaactgtgaa 1741 |
| | | aggtttggat tataaagcat tcaaacaaat tgttgaatcc tgtggtaatt |
| | | ttaaagttac |
| | | 1801 aaaaggaaaa gctaaaaaag gtgcctggaa tattggtgaa |
| | | cagaaatcaa tactgagtcc 1861 tctttatgca tttgcatcag |
| | | aggctgctcg tgttgtacga tcaattttct cccgcactct 1921 |
| | | tgaaactgct caaaattctg tgcgtgtttt acagaaggcc |
| | | gctataacaa tactagatgg 1981 aatttcacag tattcactga |
| | | gactcattga tgctatgatg ttcacatctg atttggctac 2041 |
| | | taacaatcta gttgtaatgg cctacattac aggtggtgtt |
| | | gttcagttga cttcgcagtg |
| | | 2101 gctaactaac atctttggca ctgtttatga aaaactcaaa |
| | | cccgtccttg attggcttga 2161 agagaagttt aaggaaggtg |
| | | tagagtttct tagagacggt tgggaaattg ttaaatttat 2221 |
| | | ctcaacctgt gcttgtgaaa ttgtcggtgg acaaattgtc |
| | | acctgtgcaa aggaaattaa 2281 ggagagtgtt cagacattct |
| | | ttaagcttgt aaataaattt ttggctttgt gtgctgactc 2341 |
| | | tatcattatt ggtggagcta aacttaaagc cttgaattta |
| | | ggtgaaacat ttgtcacgca |
| | | 2401 ctcaaaggga ttgtacagaa agtgtgttaa atccagagaa |
| | | gaaactggcc tactcatgcc 2461 tctaaaagcc ccaaaagaaa |
| | | ttatcttctt agagggagaa acacttccca cagaagtgtt 2521 |
| | | aacagaggaa gttgtcttga aaactggtga tttacaacca |
| | | ttagaacaac ctactagtga 2581 agctgttgaa gctccattgg |
| | | ttggtacacc agtttgtatt aacgggctta tgttgctcga 2641 |
| | | aatcaaagac acagaaaagt actgtgccct tgcacctaat |
| | | atgatggtaa caaacaatac 2701 cttcacactc aaaggcggtg |
| | | caccaacaaa ggttactttt ggtgatgaca ctgtgataga 2761 |
| | | agtgcaaggt tacaagagtg tgaatatcac ttttgaactt |
| | | gatgaaagga ttgataaagt 2821 acttaatgag aagtgctctg |
| | | cctatacagt tgaactcggt acagaagtaa atgagttcgc 2881 |
| | | ctgtgttgtg gcagatgctg tcataaaaac tttgcaacca |
| | | gtatctgaat tacttacacc |
| | | 2941 actgggcatt gatttagatg agtggagtat ggctacatac |
| | | tacttatttg atgagtctgg 3001 tgagtttaaa ttggcttcac |
| | | atatgtattg ttctttctac cctccagatg aggatgaaga 3061 |
| | | agaaggtgat tgtgaagaag aagagtttga gccatcaact |
| | | caatatgagt atggtactga 3121 agatgattac caaggtaaac |
| | | ctttggaatt tggtgccact tctgctgctc ttcaacctga 3181 |
| | | agaagagcaa gaagaagatt ggttagatga tgatagtcaa |
| | | caaactgttg gtcaacaaga 3241 cggcagtgag gacaatcaga |
| | | caactactat tcaaacaatt gttgaggttc aacctcaatt 3301 |
| | | agagatggaa cttacaccag ttgttcagac tattgaagtg |
| | | aatagtttta gtggttattt 3361 aaaacttact gacaatgtat |
| | | acattaaaaa tgcagacatt gtggaagaag ctaaaaaggt 3421 |
| | | aaaaccaaca gtggttgtta atgcagccaa tgtttacctt |
| | | aaacatggag gaggtgttgc |
| | | 3481 aggagcctta aataaggcta ctaacaatgc catgcaagtt |
| | | gaatctgatg attacatagc 3541 tactaatgga ccacttaaag |
| | | tgggtggtag ttgtgtttta agcggacaca atcttgctaa 3601 |
| | | acactgtctt catgttgtcg gcccaaatgt taacaaaggt |
| | | gaagacatto aacttcttaa 3661 gagtgcttat gaaaatttta |
| | | atcagcacga agttctactt gcaccattat tatcagctgg 3721 |
| | | tatttttggt gctgacccta tacattcttt aagagtttgt gtagatactg |
| | | ttogcacaaa |
| | | 3781 tgtctactta gctgtctttg ataaaaatct ctatgacaaa |
| | | cttgtttcaa gctttttgga 3841 aatgaagagt gaaaagcaag |
| | | ttgaacaaaa gatcgctgag attcctaaag aggaagttaa |
| | | 3901 gccatttata actgaaagta aaccttcagt tgaacagaga |
| | | aaacaagatg ataagaaaat 3961 caaagcttgt gttgaagaag |
| | | ttacaacaac tctggaagaa actaagttcc tcacagaaaa 4021 |
| | | cttgttactt tatattgaca ttaatggcaa tcttcatcca gattctgcca |
| | | ctcttgttag 4081 tgacattgac atcactttct taaagaaaga |
| | | tgctccatat atagtgggtg atgttgttca 4141 agagggtgtt |
| | | ttaactgctg tggttatacc tactaaaaag gctggtggca |
| | | ctactgaaat |
| | | 4201 gctagcgaaa gctttgagaa aagtgccaac agacaattat |
| | | ataaccactt acccgggtca 4261 gggtttaaat ggttacactg |
| | | tagaggaggc aaagacagtg cttaaaaagt gtaaaagtgc |
| | | 4321 cttttacatt ctaccatcta ttatctctaa tgagaagcaa |
| | | gaaattcttg gaactgtttc 4381 ttggaatttg cgagaaatgc |
| | | ttgcacatgc agaagaaaca cgcaaattaa tgcctgtctg |
| | | 4441 tgtggaaact aaagccatag tttcaactat acagcgtaaa |
| | | tataagggta ttaaaataca 4501 agagggtgtg gttgattatg |
| | | gtgctagatt ttacttttac accagtaaaa caactgtagc 4561 |
| | | gtcacttato aacacactta acgatctaaa tgaaactctt |
| | | gttacaatgc cacttggcta 4621 tgtaacacat ggcttaaatt |
| | | tggaagaagc tgctcggtat atgagatctc tcaaagtgcc 4681 |
| | | agctacagtt tctgtttctt cacctgatgc tgttacagcg tataatggtt |
| | | atcttacttc |
| | | 4741 ttcttctaaa acacctgaag aacattttat tgaaaccatc |
| | | tcacttgctg gttcctataa 4801 agattggtcc tattctggac |
| | | aatctacaca actaggtata gaatttctta agagaggtga |
| | | 4861 taaaagtgta tattacacta gtaatcctac cacattccac |
| | | ctagatggtg aagttatcac 4921 ctttgacaat cttaagacac |
| | | ttctttcttt gagagaagtg aggactatta aggtgtttac 4981 |
| | | aacagtagac aacattaacc tccacacgca agttgtggac |
| | | atgtcaatga catatggaca 5041 acagtttggt ccaacttatt |
| | | tggatggagc tgatgttact aaaataaaac ctcataattc 5101 |
| | | acatgaaggt aaaacatttt atgttttacc taatgatgac actctacgtg |
| | | ttgaggcttt 5161 tgagtactac cacacaactg atcctagttt |
| | | tctgggtagg tacatgtcag cattaaatca 5221 cactaaaaag |
| | | tggaaatacc cacaagttaa tggtttaact tctattaaat |
| | | gggcagataa |
| | | 5281 caactgttat cttgccactg cattgttaac actccaacaa |
| | | atagagttga agtttaatcc 5341 acctgctcta caagatgctt |
| | | attacagagc aagggctggt gaagctgcta acttttgtgc |
| | | 5401 acttatctta gcctactgta ataagacagt aggtgagtta |
| | | ggtgatgtta gagaaacaat 5461 gagttacttg tttcaacatg |
| | | ccaatttaga ttcttgcaaa agagtcttga acgtggtgtg 5521 |
| | | taaaacttgt ggacaacage agacaaccct taagggtgta |
| | | gaagctgtta tgtacatggg 5581 cacactttct tatgaacaat |
| | | ttaagaaagg tgttcagata ccttgtacgt gtggtaaaca 5641 |
| | | agctacaaaa tatctagtac aacaggagtc accttttgtt |
| | | atgatgtcag caccacctgc 5701 tcagtatgaa cttaagcatg |
| | | gtacatttac ttgtgctagt gagtacactg gtaattacca 5761 |
| | | gtgtggtcac tataaacata taacttctaa agaaactttg tattgcatag |
| | | acggtgcttt |
| | | 5821 acttacaaag tcctcagaat acaaaggtcc tattacggat |
| | | gttttctaca aagaaaacag 5881 ttacacaaca accataaaac |
| | | cagttactta taaattggat ggtgttgttt gtacagaaat 5941 |
| | | tgaccctaag ttggacaatt attataagaa agacaattct |
| | | tatttcacag agcaaccaat 6001 tgatcttgta ccaaaccaac |
| | | catatccaaa cgcaagcttc gataatttta agtttgtatg 6061 |
| | | tgataatatc aaatttgctg atgatttaaa ccagttaact ggttataaga |
| | | aacctgcttc |
| | | 6121 aagagagctt aaagttacat ttttccctga cttaaatggt |
| | | gatgtggtgg ctattgatta 6181 taaacactac acaccctctt |
| | | ttaagaaagg agctaaattg ttacataaac ctattgtttg 6241 |
| | | gcatgttaac aatgcaacta ataaagccac gtataaacca |
| | | aatacctggt gtatacgttg 6301 tctttggagc acaaaaccag |
| | | ttgaaacatc aaattcgttt gatgtactga agtcagagga 6361 |
| | | cgcgcaggga atggataatc ttgcctgcga agatctaaaa |
| | | ccagtctctg aagaagtagt 6421 ggaaaatcct accatacaga |
| | | aagacgttct tgagtgtaat gtgaaaacta ccgaagttgt 6481 |
| | | aggagacatt atacttaaac cagcaaataa tagtttaaaa |
| | | attacagaag aggttggcca 6541 cacagatcta atggctgctt |
| | | atgtagacaa ttctagtctt actattaaga aacctaatga 6601 |
| | | attatctaga gtattaggtt tgaaaaccct tgctactcat ggtttagctg |
| | | ctgttaatag |
| | | 6661 tgtcccttgg gatactatag ctaattatgc taagcctttt |
| | | cttaacaaag ttgttagtac 6721 aactactaac atagttacac |
| | | ggtgtttaaa ccgtgtttgt actaattata tgccttattt 6781 |
| | | ctttacttta ttgctacaat tgtgtacttt tactagaagt acaaattcta |
| | | gaattaaagc |
| | | 6841 atctatgccg actactatag caaagaatac tgttaagagt |
| | | gtcggtaaat tttgtctaga 6901 ggcttcattt aattatttga |
| | | agtcacctaa tttttctaaa ctgataaata ttataatttg 6961 |
| | | gtttttacta ttaagtgttt gcctaggttc tttaatctac tcaaccgctg |
| | | ctttaggtgt 7021 tttaatgtct aatttaggca tgccttctta |
| | | ctgtactggt tacagagaag gctatttgaa 7081 ctctactaat |
| | | gtcactattg caacctactg tactggttct ataccttgta gtgtttgtct |
| | | 7141 tagtggttta gattctttag acacctatcc ttctttagaa |
| | | actatacaaa ttaccatttc 7201 atcttttaaa tgggatttaa |
| | | ctgcttttgg cttagttgca gagtggtttt tggcatatat |
| | | 7261 tcttttcact aggtttttct atgtacttgg attggctgca |
| | | atcatgcaat tgtttttcag 7321 ctattttgca gtacatttta |
| | | ttagtaattc ttggcttatg tggttaataa ttaatcttgt 7381 |
| | | acaaatggcc ccgatttcag ctatggttag aatgtacatc |
| | | ttctttgcat cattttatta 7441 tgtatggaaa agttatgtgc |
| | | atgttgtaga cggttgtaat tcatcaactt gtatgatgtg 7501 |
| | | ttacaaacgt aatagagcaa caagagtcga atgtacaact |
| | | attgttaatg gtgttagaag |
| | | 7561 gtccttttat gtctatgcta atggaggtaa aggcttttgc |
| | | aaactacaca attggaattg 7621 tgttaattgt gatacattct |
| | | gtgctggtag tacatttatt agtgatgaag ttgcgagaga 7681 |
| | | cttgtcacta cagtttaaaa gaccaataaa tcctactgac cagtcttctt |
| | | acatcgttga 7741 tagtgttaca gtgaagaatg gttccatcca |
| | | tctttacttt gataaagctg gtcaaaagac 7801 ttatgaaaga |
| | | cattctctct ctcattttgt taacttagac aacctgagag ctaataacac |
| | | 7861 taaaggttca ttgcctatta atgttatagt ttttgatggt |
| | | aaatcaaaat gtgaagaatc 7921 atctgcaaaa tcagcgtctg |
| | | tttactacag tcagcttatg tgtcaaccta tactgttact 7981 |
| | | agatcaggca ttagtgtctg atgttggtga tagtgcggaa |
| | | gttgcagtta aaatgtttga 8041 tgcttacgtt aatacgtttt |
| | | catcaacttt taacgtacca atggaaaaac tcaaaacact 8101 |
| | | agttgcaact gcagaagctg aacttgcaaa gaatgtgtcc |
| | | ttagacaatg tcttatctac |
| | | 8161 ttttatttca gcagctcggc aagggtttgt tgattcagat |
| | | gtagaaacta aagatgttgt 8221 tgaatgtctt aaattgtcac |
| | | atcaatctga catagaagtt actggcgata gttgtaataa 8281 |
| | | ctatatgctc acctataaca aagttgaaaa catgacaccc |
| | | cgtgaccttg gtgcttgtat 8341 tgactgtagt gcgcgtcata |
| | | ttaatgcgca ggtagcaaaa agtcacaaca ttgctttgat 8401 |
| | | atggaacgtt aaagatttca tgtcattgtc tgaacaacta |
| | | cgaaaacaaa tacgtagtgc |
| | | 8461 tgctaaaaag aataacttac cttttaagtt gacatgtgca |
| | | actactagac aagttgttaa 8521 tgttgtaaca acaaagatag |
| | | cacttaaggg tggtaaaatt gttaataatt ggttgaagca 8581 |
| | | gttaattaaa gttacacttg tgttcctttt tgttgctgct attttctatt |
| | | taataacacc 8641 tgttcatgtc atgtctaaac atactgactt |
| | | ttcaagtgaa atcataggat acaaggctat 8701 tgatggtggt |
| | | gtcactcgtg acatagcatc tacagatact tgttttgcta |
| | | acaaacatgc |
| | | 8761 tgattttgac acatggttta gccagcgtgg tggtagttat |
| | | actaatgaca aagcttgccc 8821 attgattgct gcagtcataa |
| | | caagagaagt gggttttgtc gtgcctggtt tgcctggcac 8881 |
| | | gatattacgc acaactaatg gtgacttttt gcatttctta cctagagttt |
| | | ttagtgcagt 8941 tggtaacatc tgttacacac catcaaaact |
| | | tatagagtac actgactttg caacatcagc 9001 ttgtgttttg |
| | | gctgctgaat gtacaatttt taaagatgct tctggtaagc |
| | | cagtaccata |
| | | 9061 ttgttatgat accaatgtac tagaaggttc tgttgcttat |
| | | gaaagtttac gccctgacac 9121 acgttatgtg ctcatggatg |
| | | gctctattat tcaatttect aacacctacc ttgaaggttc 9181 |
| | | tgttagagtg gtaacaactt ttgattctga gtactgtagg |
| | | cacggcactt gtgaaagatc 9241 agaagctggt gtttgtgtat |
| | | ctactagtgg tagatgggta cttaacaatg attattacag 9301 |
| | | atctttacca ggagttttct gtggtgtaga tgctgtaaat ttacttacta |
| | | atatgtttac |
| | | 9361 accactaatt caacctattg gtgctttgga catatcagca |
| | | tctatagtag ctggtggtat 9421 tgtagctatc gtagtaacat |
| | | gccttgccta ctattttatg aggtttagaa gagcttttgg 9481 |
| | | tgaatacagt catgtagttg cctttaatac tttactattc cttatgtcat |
| | | tcactgtact 9541 ctgtttaaca ccagtttact cattcttacc |
| | | tggtgtttat tctgttattt acttgtactt 9601 gacattttat |
| | | cttactaatg atgtttcttt tttagcacat attcagtgga tggttatgtt |
| | | 9661 cacaccttta gtacctttct ggataacaat tgcttatatc |
| | | atttgtattt ccacaaagca 9721 tttctattgg ttctttagta |
| | | attacctaaa gagacgtgta gtctttaatg gtgtttcctt 9781 |
| | | tagtactttt gaagaagctg cgctgtgcac ctttttgtta |
| | | aataaagaaa tgtatctaaa |
| | | 9841 gttgcgtagt gatgtgctat tacctcttac gcaatataat |
| | | agatacttag ctctttataa 9901 taagtacaag tattttagtg |
| | | gagcaatgga tacaactagc tacagagaag ctgcttgttg 9961 |
| | | tcatctogca aaggctctca atgacttcag taactcaggt |
| | | tctgatgttc tttaccaacc 10021 accacaaacc tctatcacct |
| | | cagctgtttt gcagagtggt tttagaaaaa tggcattccc 10081 |
| | | atctggtaaa gttgagggtt gtatggtaca agtaacttgt |
| | | ggtacaacta cacttaacgg |
| | | 10141 tctttggctt gatgacgtag tttactgtcc aagacatgtg |
| | | atctgcacct ctgaagacat 10201 gottaaccct aattatgaag |
| | | atttactcat togtaagtct aatcataatt tottggtaca 10261 |
| | | ggctggtaat gttcaactca gggttattgg acattctatg |
| | | caaaattgtg tacttaagct 10321 taaggttgat acagccaatc |
| | | ctaagacacc taagtataag tttgttcgca ttcaaccagg 10381 |
| | | acagactttt tcagtgttag cttgttacaa tggttcacca tctggtgttt |
| | | accaatgtgc |
| | | 10441 tatgaggccc aatttcacta ttaagggttc attccttaat |
| | | ggttcatgtg gtagtgttgg 10501 ttttaacata gattatgact |
| | | gtgtctcttt ttgttacatg caccatatgg aattaccaac 10561 |
| | | tggagttcat gctggcacag acttagaagg taacttttat |
| | | ggaccttttg ttgacaggca 10621 aacagcacaa gcagctggta |
| | | cggacacaac tattacagtt aatgttttag cttggttgta 10681 |
| | | cgctgctgtt ataaatggag acaggtggtt tctcaatcga |
| | | tttaccacaa ctcttaatga |
| | | 10741 ctttaacctt gtggctatga agtacaatta tgaacctcta |
| | | acacaagacc atgttgacat 10801 actaggacct ctttctgctc |
| | | aaactggaat tgccgtttta gatatgtgtg cttcattaaa 10861 |
| | | agaattactg caaaatggta tgaatggacg taccatattg |
| | | ggtagtgctt tattagaaga 10921 tgaatttaca ccttttgatg |
| | | ttgttagaca atgctcaggt gttactttcc aaagtgcagt 10981 |
| | | gaaaagaaca atcaagggta cacaccactg gttgttactc |
| | | acaattttga cttcactttt |
| | | 11041 agttttagtc cagagtactc aatggtcttt gttctttttt |
| | | ttgtatgaaa atgccttttt 11101 accttttgct atgggtatta |
| | | ttgctatgtc tgcttttgca atgatgtttg tcaaacataa 11161 |
| | | gcatgcattt ctctgtttgt ttttgttacc ttctcttgcc actgtagctt |
| | | attttaatat 11221 ggtctatatg cctgctagtt gggtgatgcg |
| | | tattatgaca tggttggata tggttgatac 11281 tagtttgtct |
| | | ggttttaagc taaaagactg tgttatgtat gcatcagctg tagtgttact |
| | | 11341 aatccttatg acagcaagaa ctgtgtatga tgatggtgct |
| | | aggagagtgt ggacacttat 11401 gaatgtcttg acactcgttt |
| | | ataaagttta ttatggtaat gctttagatc aagccatttc 11461 |
| | | catgtgggct cttataatct ctgttacttc taactactca ggtgtagtta |
| | | caactgtcat 11521 gtttttggcc agaggtattg tttttatgtg |
| | | tgttgagtat tgccctattt tottcataac 11581 tggtaataca |
| | | cttcagtgta taatgctagt ttattgtttc ttaggctatt tttgtacttg |
| | | 11641 ttactttggc ctcttttgtt tactcaaccg ctactttaga |
| | | ctgactcttg gtgtttatga 11701 ttacttagtt tctacacagg |
| | | agtttagata tatgaattca cagggactac tcccacccaa 11761 |
| | | gaatagcata gatgccttca aactcaacat taaattgttg |
| | | ggtgttggtg gcaaaccttg 11821 tatcaaagta gccactgtac |
| | | agtctaaaat gtcagatgta aagtgcacat cagtagtctt 11881 |
| | | actctcagtt ttgcaacaac tcagagtaga atcatcatct |
| | | aaattgtggg ctcaatgtgt |
| | | 11941 ccagttacac aatgacatte tcttagctaa agatactact |
| | | gaagcctttg aaaaaatggt 12001 ttcactactt tctgttttgc |
| | | tttccatgca gggtgctgta gacataaaca agctttgtga 12061 |
| | | agaaatgctg gacaacaggg caaccttaca agctatagcc |
| | | tcagagttta gttcccttcc 12121 atcatatgca gottttgcta |
| | | ctgctcaaga agcttatgag caggctgttg ctaatggtga 12181 |
| | | ttctgaagtt gttcttaaaa agttgaagaa gtctttgaat gtggctaaat |
| | | ctgaatttga 12241 ccgtgatgca gccatgcaac gtaagttgga |
| | | aaagatggct gatcaagcta tgacccaaat 12301 gtataaacag |
| | | gctagatctg aggacaagag ggcaaaagtt actagtgcta |
| | | tgcagacaat 12361 gcttttcact atgcttagaa agttggataa |
| | | tgatgcactc aacaacatta tcaacaatgc 12421 aagagatggt |
| | | tgtgttccct tgaacataat acctcttaca acagcagcca |
| | | aactaatggt |
| | | 12481 tgtcatacca gactataaca catataaaaa tacgtgtgat |
| | | ggtacaacat ttacttatgc 12541 atcagcattg tgggaaatcc |
| | | aacaggttgt agatgcagat agtaaaattg ttcaacttag 12601 |
| | | tgaaattagt atggacaatt cacctaattt agcatggcct cttattgtaa |
| | | cagctttaag 12661 ggccaattct gctgtcaaat tacagaataa |
| | | tgagcttagt cctgttgcac tacgacagat 12721 gtcttgtgct |
| | | gccggtacta cacaaactgc ttgcactgat gacaatgcgt |
| | | tagcttacta |
| | | 12781 caacacaaca aagggaggta ggtttgtact tgcactgtta |
| | | tccgatttac aggatttgaa 12841 atgggctaga ttccctaaga |
| | | gtgatggaac tggtactate tatacagaac tggaaccacc 12901 |
| | | ttgtaggttt gttacagaca cacctaaagg tcctaaagtg |
| | | aagtatttat actttattaa 12961 aggattaaac aacctaaata |
| | | gaggtatggt acttggtagt ttagctgcca cagtacgtct 13021 |
| | | acaagctggt aatgcaacag aagtgcctgc caattcaact |
| | | gtattatctt tctgtgcttt |
| | | 13081 tgctgtagat gctgctaaag cttacaaaga ttatctagct |
| | | agtgggggac aaccaatcac 13141 taattgtgtt aagatgttgt |
| | | gtacacacac tggtactggt caggcaataa cagttacacc 13201 |
| | | ggaagccaat atggatcaag aatcctttgg tggtgcatcg |
| | | tgttgtctgt actgccgttg 13261 ccacatagat catccaaatc |
| | | ctaaaggatt ttgtgactta aaaggtaagt atgtacaaat 13321 |
| | | acctacaact tgtgctaatg accctgtggg ttttacactt |
| | | aaaaacacag tctgtaccgt |
| | | 13381 ctgcggtatg tggaaaggtt atggctgtag ttgtgatcaa |
| | | ctccgcgaac ccatgcttca 13441 gtcagctgat gcacaatcgt |
| | | ttttaaacgg gtttgcggtg taagtgcage ccgtcttaca 13501 |
| | | ccgtgoggca caggcactag tactgatgtc gtatacaggg |
| | | cttttgacat ctacaatgat 13561 aaagtagctg gttttgctaa |
| | | attcctaaaa actaattgtt gtogcttcca agaaaaggac |
| | | 13621 gaagatgaca atttaattga ttcttacttt gtagttaaga |
| | | gacacacttt ctctaactac 13681 caacatgaag aaacaattta |
| | | taatttactt aaggattgtc cagctgttgc taaacatgac 13741 |
| | | ttctttaagt ttagaataga cggtgacatg gtaccacata |
| | | tatcacgtca acgtcttact 13801 aaatacacaa tggcagacct |
| | | cgtctatgct ttaaggcatt ttgatgaagg taattgtgac 13861 |
| | | acattaaaag aaatacttgt cacatacaat tgttgtgatg atgattattt |
| | | caataaaaag |
| | | 13921 gactggtatg attttgtaga aaacccagat atattacgcg |
| | | tatacgccaa cttaggtgaa 13981 cgtgtacgcc aagctttgtt |
| | | aaaaacagta caattctgtg atgccatgcg aaatgctggt 14041 |
| | | attgttggtg tactgacatt agataatcaa gatctcaatg |
| | | gtaactggta tgatttoggt 14101 gatttcatac aaaccacgcc |
| | | aggtagtgga gttcctgttg tagattctta ttattcattg 14161 |
| | | ttaatgccta tattaacctt gaccagggct ttaactgcag |
| | | agtcacatgt tgacactgac |
| | | 14221 ttaacaaagc cttacattaa gtgggatttg ttaaaatatg |
| | | acttcacgga agagaggtta 14281 aaactctttg accgttattt |
| | | taaatattgg gatcagacat accacccaaa ttgtgttaac 14341 |
| | | tgtttggatg acagatgcat tctgcattgt gcaaacttta atgttttatt |
| | | ctctacagtg 14401 ttcccaccta caagttttgg accactagtg |
| | | agaaaaatat ttgttgatgg tgttccattt 14461 gtagtttcaa |
| | | ctggatacca cttcagagag ctaggtgttg tacataatca |
| | | ggatgtaaac |
| | | 14521 ttacatagct ctagacttag ttttaaggaa ttacttgtgt |
| | | atgctgctga ccctgctatg 14581 cacgctgctt ctggtaatct |
| | | attactagat aaacgcacta cgtgcttttc agtagctgca 14641 |
| | | cttactaaca atgttgcttt tcaaactgtc aaacccggta attttaacaa |
| | | agacttctat 14701 gactttgctg tgtctaaggg tttctttaag |
| | | gaaggaagtt ctgttgaatt aaaacacttc 14761 ttctttgctc |
| | | aggatggtaa tgctgctatc agcgattatg actactatcg |
| | | ttataatcta |
| | | 14821 ccaacaatgt gtgatatcag acaactacta tttgtagttg |
| | | aagttgttga taagtacttt 14881 gattgttacg atggtggctg |
| | | tattaatgct aaccaagtca togtcaacaa cctagacaaa 14941 |
| | | tcagctggtt ttccatttaa taaatggggt aaggctagac tttattatga |
| | | ttcaatgagt 15001 tatgaggatc aagatgcact tttcgcatat |
| | | acaaaacgta atgtcatccc tactataact 15061 caaatgaatc |
| | | ttaagtatgc cattagtgca aagaatagag ctcgcaccgt |
| | | agctggtgtc 15121 tctatctgta gtactatgac caatagacag |
| | | tttcatcaaa aattattgaa atcaatagcc 15181 gccactagag |
| | | gagctactgt agtaattgga acaagcaaat tctatggtgg |
| | | ttggcacaac 15241 atgttaaaaa ctgtttatag tgatgtagaa |
| | | aaccctcacc ttatgggttg ggattatcct 15301 aaatgtgata |
| | | gagccatgcc taacatgctt agaattatgg cctcacttgt |
| | | tcttgctcgc |
| | | 15361 aaacatacaa cgtgttgtag cttgtcacac cgtttctata |
| | | gattagctaa tgagtgtgct 15421 caagtattga gtgaaatggt |
| | | catgtgtggc ggttcactat atgttaaacc aggtggaacc 15481 |
| | | tcatcaggag atgccacaac tgcttatgct aatagtgttt ttaacatttg |
| | | tcaagctgtc 15541 acggccaatg ttaatgcact tttatctact |
| | | gatggtaaca aaattgccga taagtatgtc 15601 cgcaatttac |
| | | aacacagact ttatgagtgt ctctatagaa atagagatgt |
| | | tgacacagac |
| | | 15661 tttgtgaatg agttttacgc atatttgcgt aaacatttct |
| | | caatgatgat actctctgac 15721 gatgctgttg tgtgtttcaa |
| | | tagcacttat gcatctcaag gtctagtggc tagcataaag 15781 |
| | | aactttaagt cagttcttta ttatcaaaac aatgttttta tgtctgaagc |
| | | aaaatgttgg 15841 actgagactg accttactaa aggacctcat |
| | | gaattttgct ctcaacatac aatgctagtt 15901 aaacagggtg |
| | | atgattatgt gtaccttoct tacccagato catcaagaat |
| | | cctaggggcc |
| | | 15961 ggctgttttg tagatgatat cgtaaaaaca gatggtacac |
| | | ttatgattga acggttcgtg 16021 tctttagcta tagatgctta |
| | | cccacttact aaacatccta atcaggagta tgctgatgtc 16081 |
| | | tttcatttgt acttacaata cataagaaag ctacatgatg |
| | | agttaacagg acacatgtta 16141 gacatgtatt ctgttatgct |
| | | tactaatgat aacacttcaa ggtattggga acctgagttt 16201 |
| | | tatgaggcta tgtacacacc gcatacagtc ttacaggctg |
| | | ttggggcttg tgttctttgc |
| | | 16261 aattcacaga cttcattaag atgtggtgct tgcatacgta |
| | | gaccattctt atgttgtaaa 16321 tgctgttacg accatgtcat |
| | | atcaacatca cataaattag tottgtctgt taatccgtat 16381 |
| | | gtttgcaatg ctccaggttg tgatgtcaca gatgtgactc aactttactt |
| | | aggaggtatg 16441 agctattatt gtaaatcaca taaaccaccc |
| | | attagttttc cattgtgtgc taatggacaa 16501 gtttttggtt |
| | | tatataaaaa tacatgtgtt ggtagcgata atgttactga ctttaatgca |
| | | 16561 attgcaacat gtgactggac aaatgctggt gattacattt |
| | | tagctaacac ctgtactgaa 16621 agactcaagc tttttgcagc |
| | | agaaacgctc aaagctactg aggagacatt taaactgtct 16681 |
| | | tatggtattg ctactgtacg tgaagtgctg tctgacagag |
| | | aattacatct ttcatgggaa 16741 gttggtaaac ctagaccacc |
| | | acttaaccga aattatgtct ttactggtta togtgtaact 16801 |
| | | aaaaacagta aagtacaaat aggagagtac acctttgaaa |
| | | aaggtgacta tggtgatgct 16861 gttgtttacc gaggtacaac |
| | | aacttacaaa ttaaatgttg gtgattattt tgtgctgaca 16921 |
| | | tcacatacag taatgccatt aagtgcacct acactagtgc |
| | | cacaagagca ctatgttaga |
| | | 16981 attactggct tatacccaac actcaatatc tcagatgagt |
| | | tttctagcaa tgttgcaaat 17041 tatcaaaagg ttggtatgca |
| | | aaagtattct acactccagg gaccacctgg tactggtaag |
| | | 17101 agtcattttg ctattggcct agctctctac tacccttctg |
| | | ctogcatagt gtatacagct 17161 tgctctcatg ccgctgttga |
| | | tgcactatgt gagaaggcat taaaatattt gcctatagat 17221 |
| | | aaatgtagta gaattatacc tgcacgtgct cgtgtagagt |
| | | gttttgataa attcaaagtg 17281 aattcaacat tagaacagta |
| | | tgtcttttgt actgtaaatg cattgcctga gacgacagca 17341 |
| | | gatatagttg tctttgatga aatttcaatg gccacaaatt atgatttgag |
| | | tgttgtcaat |
| | | 17401 gccagattac gtgctaagca ctatgtgtac attggcgacc |
| | | ctgctcaatt acctgcacca 17461 cgcacattgc taactaaggg |
| | | cacactagaa ccagaatatt tcaattcagt gtgtagactt 17521 |
| | | atgaaaacta taggtccaga catgttcctc ggaacttgtc |
| | | ggcgttgtcc tgctgaaatt 17581 gttgacactg tgagtgcttt |
| | | ggtttatgat aataagctta aagcacataa agacaaatca 17641 |
| | | gctcaatgct ttaaaatgtt ttataagggt gttatcacgc atgatgtttc |
| | | atctgcaatt |
| | | 17701 aacaggccac aaataggcgt ggtaagagaa ttccttacac |
| | | gtaaccctgc ttggagaaaa 17761 gctgtcttta tttcacctta |
| | | taattcacag aatgctgtag cctcaaagat tttgggacta |
| | | 17821 ccaactcaaa ctgttgatt atcacagggc tcagaatatg |
| | | actatgtcat attcactcaa 17881 accactgaaa cagctcactc |
| | | ttgtaatgta aacagattta atgttgctat taccagagca 17941 |
| | | aaagtaggca tactttgcat aatgtctgat agagaccttt |
| | | atgacaagtt gcaatttaca 18001 agtcttgaaa ttccacgtag |
| | | gaatgtggca actttacaag ctgaaaatgt aacaggactc 18061 |
| | | tttaaagatt gtagtaaggt aatcactggg ttacatccta |
| | | cacaggcacc tacacacctc 18121 agtgttgaca ctaaattcaa |
| | | aactgaaggt ttatgtgttg acatacctgg catacctaag 18181 |
| | | gacatgacct atagaagact catctctatg atgggtttta |
| | | aaatgaatta tcaagttaat |
| | | 18241 ggttacccta acatgtttat cacccgcgaa gaagctataa |
| | | gacatgtacg tgcatggatt 18301 ggcttcgatg tcgaggggtg |
| | | tcatgctact agagaagctg ttggtaccaa tttaccttta 18361 |
| | | cagctaggtt tttctacagg tgttaaccta gttgctgtac ctacaggtta |
| | | tgttgataca 18421 cctaataata cagatttttc cagagttagt |
| | | gctaaaccac cgcctggaga tcaatttaaa 18481 cacctcatac |
| | | cacttatgta caaaggactt ccttggaatg tagtgcgtat |
| | | aaagattgta |
| | | 18541 caaatgttaa gtgacacact taaaaatctc tctgacagag |
| | | tcgtatttgt cttatgggca 18601 catggctttg agttgacatc |
| | | tatgaagtat tttgtgaaaa taggacctga gcgcacctgt |
| | | 18661 tgtctatgtg atagacgtgc cacatgcttt tccactgctt |
| | | cagacactta tgcctgttgg 18721 catcattcta ttggatttga |
| | | ttacgtctat aatccgttta tgattgatgt tcaacaatgg 18781 |
| | | ggttttacag gtaacctaca aagcaaccat gatctgtatt |
| | | gtcaagtcca tggtaatgca 18841 catgtagcta gttgtgatgc |
| | | aatcatgact aggtgtctag ctgtccacga gtgctttgtt 18901 |
| | | aagcgtgttg actggactat tgaatatcct ataattggtg |
| | | atgaactgaa gattaatgcg |
| | | 18961 gcttgtagaa aggttcaaca catggttgtt aaagctgcat |
| | | tattagcaga caaattccca 19021 gttcttcacg acattggtaa |
| | | ccctaaagct attaagtgtg tacctcaagc tgatgtagaa |
| | | 19081 tggaagttct atgatgcaca gccttgtagt gacaaagctt |
| | | ataaaataga agaattattc 19141 tattcttatg ccacacattc |
| | | tgacaaattc acagatggtg tatgcctatt ttggaattgc 19201 |
| | | aatgtcgata gatatcctgc taattccatt gtttgtagat ttgacactag |
| | | agtgctatct 19261 aaccttaact tgcctggttg tgatggtggc |
| | | agtttgtatg taaataaaca tgcattccac 19321 acaccagctt |
| | | ttgataaaag tgcttttgtt aatttaaaac aattaccatt tttctattac |
| | | 19381 tctgacagtc catgtgagtc tcatggaaaa caagtagtgt |
| | | cagatataga ttatgtacca 19441 ctaaagtctg ctacgtgtat |
| | | aacacgttgc aatttaggtg gtgctgtctg tagacatcat 19501 |
| | | gctaatgagt acagattgta tctcgatgct tataacatga |
| | | tgatctcagc tggctttagc 19561 ttgtgggttt acaaacaatt |
| | | tgatacttat aacctctgga acacttttac aagacttcag 19621 |
| | | agtttagaaa atgtggcttt taatgttgta aataagggac |
| | | actttgatgg acaacagggt |
| | | 19681 gaagtaccag tttctatcat taataacact gtttacacaa |
| | | aagttgatgg tgttgatgta 19741 gaattgtttg aaaataaaac |
| | | aacattacct gttaatgtag catttgagct ttgggctaag 19801 |
| | | cgcaacatta aaccagtacc agaggtgaaa atactcaata |
| | | atttgggtgt ggacattgct 19861 gctaatactg tgatctggga |
| | | ctacaaaaga gatgctccag cacatatato tactattggt 19921 |
| | | gtttgttcta tgactgacat agccaagaaa ccaactgaaa |
| | | cgatttgtgc accactcact 19981 gtcttttttg atggtagagt |
| | | tgatggtcaa gtagacttat ttagaaatgc ccgtaatggt 20041 |
| | | gttcttatta cagaaggtag tgttaaaggt ttacaaccat |
| | | ctgtaggtcc caaacaagct 20101 agtcttaatg gagtcacatt |
| | | aattggagaa gccgtaaaaa cacagttcaa ttattataag 20161 |
| | | aaagttgatg gtgttgtcca acaattacct gaaacttact |
| | | ttactcagag tagaaattta |
| | | 20221 caagaattta aacccaggag tcaaatggaa attgatttct |
| | | tagaattagc tatggatgaa 20281 ttcattgaac ggtataaatt |
| | | agaaggctat gccttcgaac atatcgttta tggagatttt 20341 |
| | | agtcatagtc agttaggtgg tttacatcta ctgattggac |
| | | tagctaaacg ttttaaggaa 20401 tcaccttttg aattagaaga |
| | | ttttattcct atggacagta cagttaaaaa ctatttcata 20461 |
| | | acagatgcgc aaacaggttc atctaagtgt gtgtgttctg ttattgattt |
| | | attacttgat |
| | | 20521 gattttgttg aaataataaa atcccaagat ttatctgtag |
| | | tttctaaggt tgtcaaagtg 20581 actattgact atacagaaat |
| | | ttcatttatg ctttggtgta aagatggcca tgtagaaaca 20641 |
| | | ttttacccaa aattacaatc tagtcaagog tggcaaccgg |
| | | gtgttgctat gcctaatctt 20701 tacaaaatgc aaagaatgct |
| | | attagaaaag tgtgaccttc aaaattatgg tgatagtgca 20761 |
| | | acattaccta aaggcataat gatgaatgtc gcaaaatata |
| | | ctcaactgtg tcaatattta |
| | | 20821 aacacattaa cattagctgt accctataat atgagagtta |
| | | tacattttgg tgctggttct 20881 gataaaggag ttgcaccagg |
| | | tacagctgtt ttaagacagt ggttgcctac gggtacgctg 20941 |
| | | cttgtcgatt cagatcttaa tgactttgtc tctgatgcag attcaacttt |
| | | gattggtgat 21001 tgtgcaactg tacatacagc taataaatgg |
| | | gatctcatta ttagtgatat gtacgaccct 21061 aagactaaaa |
| | | atgttacaaa agaaaatgac tctaaagagg gttttttcac ttacatttgt |
| | | 21121 gggtttatac aacaaaagct agctcttgga ggttccgtgg |
| | | ctataaagat aacagaacat 21181 tcttggaatg ctgatcttta |
| | | taagctcatg ggacacttcg catggtggac agcctttgtt |
| | | 21241 actaatgtga atgcgtcatc atctgaagca tttttaattg |
| | | gatgtaatta tcttggcaaa 21301 ccacgcgaac aaatagatgg |
| | | ttatgtcatg catgcaaatt acatattttg gaggaataca 21361 |
| | | aatccaattc agttgtcttc ctattcttta tttgacatga gtaaatttcc |
| | | ccttaaatta 21421 aggggtactg ctgttatgtc tttaaaagaa |
| | | ggtcaaatca atgatatgat tttatctctt 21481 cttagtaaag |
| | | gtagacttat aattagagaa aacaacagag ttgttatttc tagtgatgtt |
| | | 21541 cttgttaaca actaaacgaa caatgtttgt ttttcttgtt |
| | | ttattgccac tagtctctag |
| | | 21601 tcagtgtgtt aatcttacaa ccagaactca attaccccct |
| | | gcatacacta attctttcac 21661 acgtggtgtt tattaccctg |
| | | acaaagtttt cagatcctca gttttacatt caactcagga 21721 |
| | | cttgttctta cctttctttt ccaatgttac ttggttccat gctatacatg |
| | | tctctgggac 21781 caatggtact aagaggtttg ataaccctgt |
| | | cctaccattt aatgatggtg tttattttgc 21841 ttccactgag |
| | | aagtctaaca taataagagg ctggattttt ggtactactt |
| | | tagattogaa |
| | | 21901 gacccagtcc ctacttattg ttaataacgc tactaatgtt |
| | | gttattaaag tctgtgaatt 21961 tcaattttgt aatgatccat |
| | | ttttgggtgt ttattaccac aaaaacaaca aaagttggat 22021 |
| | | ggaaagtgag ttcagagttt attctagtgc gaataattgc |
| | | acttttgaat atgtctctca 22081 gccttttctt atggaccttg |
| | | aaggaaaaca gggtaatttc aaaaatctta gggaatttgt 22141 |
| | | gtttaagaat attgatggtt attttaaaat atattctaag cacacgccta |
| | | ttaatttagt |
| | | 22201 gcgtgatctc cctcagggtt tttcggcttt agaaccattg |
| | | gtagatttgc caataggtat 22261 taacatcact aggtttcaaa |
| | | ctttacttgc tttacataga agttatttga ctcctggtga 22321 |
| | | ttcttcttca ggttggacag ctggtgctgc agcttattat gtgggttatc |
| | | ttcaacctag 22381 gacttttcta ttaaaatata atgaaaatgg |
| | | aaccattaca gatgctgtag actgtgcact 22441 tgaccctctc |
| | | tcagaaacaa agtgtacgtt gaaatccttc actgtagaaa |
| | | aaggaatcta |
| | | 22501 tcaaacttct aactttagag tocaaccaac agaatctatt |
| | | gttagatttc ctaatattac 22561 aaacttgtgc ccttttggtg |
| | | aagtttttaa cgccaccaga tttgcatctg tttatgcttg 22621 |
| | | gaacaggaag agaatcagca actgtgttgc tgattattct |
| | | gtcctatata attccgcatc 22681 attttccact tttaagtgtt |
| | | atggagtgtc tcctactaaa ttaaatgato tctgctttac 22741 |
| | | taatgtctat gcagattcat ttgtaattag aggtgatgaa |
| | | gtcagacaaa tcgctccagg 22801 gcaaactgga aagattgctg |
| | | attataatta taaattacca gatgatttta caggctgcgt 22861 |
| | | tatagcttgg aattctaaca atcttgattc taaggttggt ggtaattata |
| | | attacctgta |
| | | 22921 tagattgttt aggaagtcta atctcaaacc ttttgagaga |
| | | gatatttcaa ctgaaatcta 22981 tcaggccggt agcacacctt |
| | | gtaatggtgt tgaaggtttt aattgttact ttcctttaca 23041 |
| | | atcatatggt ttccaaccca ctaatggtgt tggttaccaa |
| | | ccatacagag tagtagtact 23101 ttcttttgaa cttctacatg |
| | | caccagcaac tgtttgtgga cctaaaaagt ctactaattt 23161 |
| | | ggttaaaaac aaatgtgtca atttcaactt caatggttta |
| | | acaggcacag gtgttcttac |
| | | 23221 tgagtctaac aaaaagtttc tgcctttcca acaatttggc |
| | | agagacattg ctgacactac 23281 tgatgctgtc cgtgatccac |
| | | agacacttga gattcttgac attacaccat gttcttttgg 23341 |
| | | tggtgtcagt gttataacac caggaacaaa tacttctaac |
| | | caggttgctg ttctttatca 23401 ggatgttaac tgcacagaag |
| | | tccctgttgc tattcatgca gatcaactta ctcctacttg 23461 |
| | | gcgtgtttat tctacaggtt ctaatgtttt tcaaacacgt gcaggctgtt |
| | | taataggggc |
| | | 23521 tgaacatgtc aacaactcat atgagtgtga catacccatt |
| | | ggtgcaggta tatgcgctag 23581 ttatcagact cagactaatt |
| | | ctcctcggcg ggcacgtagt gtagctagtc aatccatcat 23641 |
| | | tgcctacact atgtcacttg gtgcagaaaa ttcagttgct tactctaata |
| | | actctattgc 23701 catacccaca aattttacta ttagtgttac |
| | | cacagaaatt ctaccagtgt ctatgaccaa 23761 gacatcagta |
| | | gattgtacaa tgtacatttg tggtgattca actgaatgca gcaatctttt |
| | | 23821 gttgcaatat ggcagttttt gtacacaatt aaaccgtgct |
| | | ttaactggaa tagctgttga 23881 acaagacaaa aacacccaag |
| | | aagtttttgc acaagtcaaa caaatttaca aaacaccacc 23941 |
| | | aattaaagat tttggtggtt ttaatttttc acaaatatta ccagatccat |
| | | caaaaccaag 24001 caagaggtca tttattgaag atctactttt |
| | | caacaaagtg acacttgcag atgctggctt 24061 catcaaacaa |
| | | tatggtgatt gccttggtga tattgctgct agagacctca |
| | | tttgtgcaca |
| | | 24121 aaagtttaac ggccttactg ttttgccacc tttgctcaca |
| | | gatgaaatga ttgctcaata 24181 cacttctgca ctgttagcgg |
| | | gtacaatcac ttctggttgg acctttggtg caggtgctgc 24241 |
| | | attacaaata ccatttgcta tgcaaatggc ttataggttt aatggtattg |
| | | gagttacaca 24301 gaatgttctc tatgagaacc aaaaattgat |
| | | tgccaaccaa tttaatagtg ctattggcaa 24361 aattcaagac |
| | | tcactttctt ccacagcaag tgcacttgga aaacttcaag |
| | | atgtggtcaa |
| | | 24421 ccaaaatgca caagctttaa acacgcttgt taaacaactt |
| | | agctccaatt ttggtgcaat 24481 ttcaagtgtt ttaaatgata |
| | | tcctttcacg tcttgacaaa gttgaggctg aagtgcaaat 24541 |
| | | tgataggttg atcacaggca gacttcaaag tttgcagaca |
| | | tatgtgactc aacaattaat 24601 tagagctgca gaaatcagag |
| | | cttctgctaa tcttgctgct actaaaatgt cagagtgtgt 24661 |
| | | acttggacaa tcaaaaagag ttgatttttg tggaaagggc |
| | | tatcatctta tgtccttccc |
| | | 24721 tcagtcagca cctcatggtg tagtcttctt gcatgtgact |
| | | tatgtccctg cacaagaaaa 24781 gaacttcaca actgctcctg |
| | | ccatttgtca tgatggaaaa gcacactttc ctcgtgaagg |
| | | 24841 tgtctttgtt tcaaatggca cacactggtt tgtaacacaa |
| | | aggaattttt atgaaccaca 24901 aatcattact acagacaaca |
| | | catttgtgtc tggtaactgt gatgttgtaa taggaattgt 24961 |
| | | caacaacaca gtttatgatc ctttgcaacc tgaattagac |
| | | tcattcaagg aggagttaga 25021 taaatatttt aagaatcata |
| | | catcaccaga tgttgattta ggtgacatct ctggcattaa 25081 |
| | | tgcttcagtt gtaaacatto aaaaagaaat tgaccgcctc |
| | | aatgaggttg ccaagaattt |
| | | 25141 aaatgaatct ctcategatc tocaagaact tggaaagtat |
| | | gagcagtata taaaatggcc 25201 atggtacatt tggctaggtt |
| | | ttatagctgg cttgattgcc atagtaatgg tgacaattat 25261 |
| | | gctttgctgt atgaccagtt gctgtagttg tctcaagggc tgttgttctt |
| | | gtggatcctg 25321 ctgcaaattt gatgaagacg actctgagcc |
| | | agtgctcaaa ggagtcaaat tacattacac 25381 ataaacgaac |
| | | ttatggattt gtttatgaga atcttcacaa ttggaactgt aactttgaag |
| | | 25441 caaggtgaaa tcaaggatgc tactccttca gattttgttc |
| | | gcgctactgc aacgataccg 25501 atacaagcct cactcccttt |
| | | cggatggctt attgttggcg ttgcacttct tgctgttttt 25561 |
| | | cagagcgctt ccaaaatcat aaccctcaaa aagagatggc |
| | | aactagcact ctccaagggt 25621 gttcactttg tttgcaactt |
| | | gctgttgttg tttgtaacag tttactcaca ccttttgctc 25681 |
| | | gttgctgctg gccttgaagc cccttttctc tatctttatg ctttagtcta |
| | | cttcttgcag 25741 agtataaact ttgtaagaat aataatgagg |
| | | ctttggcttt gctggaaatg cogttccaaa 25801 aacccattac |
| | | tttatgatgc caactatttt ctttgctggc atactaattg ttacgactat |
| | | 25861 tgtatacctt acaatagtgt aacttcttca attgtcatta |
| | | cttcaggtga tggcacaaca 25921 agtcctattt ctgaacatga |
| | | ctaccagatt ggtggttata ctgaaaaatg ggaatctgga 25981 |
| | | gtaaaagact gtgttgtatt acacagttac ttcacttcag actattacca |
| | | gctgtactca 26041 actcaattga gtacagacac tggtgttgaa |
| | | catgttacct tcttcatcta caataaaatt 26101 gttgatgagc |
| | | ctgaagaaca tgtccaaatt cacacaateg acggttcatc |
| | | cggagttgtt |
| | | 26161 aatccagtaa tggaaccaat ttatgatgaa ccgacgacga |
| | | ctactagcgt gcctttgtaa 26221 gcacaagctg atgagtacga |
| | | acttatgtac tcattogttt cggaagagac aggtacgtta 26281 |
| | | atagttaata gcgtacttct ttttcttgct ttcgtggtat tcttgctagt |
| | | tacactagcc 26341 atccttactg cgcttcgatt gtgtgcgtac |
| | | tgctgcaata ttgttaacgt gagtcttgta 26401 aaaccttctt |
| | | tttacgttta ctctogtgtt aaaaatctga attcttctag agttcctgat |
| | | 26461 cttctggtct aaacgaacta aatattatat tagtttttct |
| | | gtttggaact ttaattttag 26521 ccatggcaga ttccaacggt |
| | | actattaccg ttgaagagct taaaaagctc cttgaacaat 26581 |
| | | ggaacctagt aataggtttc ctattcctta catggatttg tcttctacaa |
| | | tttgcctatg 26641 ccaacaggaa taggtttttg tatataatta |
| | | agttaatttt cctctggctg ttatggccag 26701 taactttagc |
| | | ttgttttgtg cttgctgctg tttacagaat aaattggatc accggtggaa |
| | | 26761 ttgctatcgc aatggcttgt cttgtaggct tgatgtggct |
| | | cagctacttc attgcttctt 26821 tcagactgtt tgcgcgtacg |
| | | cgttccatgt ggtcattcaa tocagaaact aacattcttc 26881 |
| | | tcaacgtgcc actccatggc actattctga ccagaccgct |
| | | tctagaaagt gaactcgtaa 26941 toggagctgt gatccttcgt |
| | | ggacatcttc gtattgctgg acaccatcta ggacgctgtg 27001 |
| | | acatcaagga cctgcctaaa gaaatcactg ttgctacatc |
| | | acgaacgctt tcttattaca |
| | | 27061 aattgggagc ttcgcagcgt gtagcaggtg actcaggttt |
| | | tgctgcatac agtcgctaca 27121 ggattggcaa ctataaatta |
| | | aacacagacc attccagtag cagtgacaat attgctttgc 27181 |
| | | ttgtacagta agtgacaaca gatgtttcat ctcgttgact ttcaggttac |
| | | tatagcagag 27241 atattactaa ttattatgag gacttttaaa |
| | | gtttccattt ggaatcttga ttacatcata 27301 aacctcataa |
| | | ttaaaaattt atctaagtca ctaactgaga ataaatattc tcaattagat |
| | | 27361 gaagagcaac caatggagat tgattaaacg aacatgaaaa |
| | | ttattctttt cttggcactg 27421 ataacactcg ctacttgtga |
| | | gctttatcac taccaagagt gtgttagagg tacaacagta 27481 |
| | | cttttaaaag aaccttgctc ttctggaaca tacgagggca |
| | | attcaccatt tcatcctcta 27541 gctgataaca aatttgcact |
| | | gacttgcttt agcactcaat ttgcttttgc ttgtcctgac 27601 |
| | | ggcgtaaaac acgtctatca gttacgtgcc agatcagttt |
| | | cacctaaact gttcatcaga |
| | | 27661 caagaggaag ttcaagaact ttactctcca atttttctta |
| | | ttgttgcggc aatagtgttt 27721 ataacacttt gcttcacact |
| | | caaaagaaag acagaatgat tgaactttca ttaattgact 27781 |
| | | tctatttgtg ctttttagcc tttctgctat tccttgtttt aattatgctt |
| | | attatctttt 27841 ggttctcact tgaactgcaa gatcataatg |
| | | aaacttgtca cgcctaaacg aacatgaaat 27901 ttcttgtttt |
| | | cttaggaatc atcacaactg tagctgcatt tcaccaagaa |
| | | tgtagtttac |
| | | 27961 agtcatgtac tcaacatcaa ccatatgtag ttgatgaccc |
| | | gtgtcctatt cacttctatt 28021 ctaaatggta tattagagta |
| | | ggagctagaa aatcagcacc tttaattgaa ttgtgcgtgg 28081 |
| | | atgaggctgg ttctaaatca cccattcagt acatcgatat |
| | | cggtaattat acagtttcct 28141 gtttaccttt tacaattaat |
| | | tgccaggaac ctaaattggg tagtcttgta gtgcgttgtt 28201 |
| | | cgttctatga agacttttta gagtatcatg acgttcgtgt tgttttagat |
| | | ttcatctaaa |
| | | 28261 cgaacaaact aaaatgtctg ataatggacc ccaaaatcag |
| | | cgaaatgcac cccgcattac 28321 gtttggtgga ccctcagatt |
| | | caactggcag taaccagaat ggagaacgca gtggggcgcg |
| | | 28381 atcaaaacaa cgteggcccc aaggtttacc caataatact |
| | | gcgtcttggt tcaccgctct 28441 cactcaacat ggcaaggaag |
| | | accttaaatt ccctogagga caagggttc caattaacac 28501 |
| | | caatagcagt ccagatgacc aaattggcta ctaccgaaga |
| | | gctaccagac gaattcgtgg 28561 tggtgacggt aaaatgaaag |
| | | atctcagtcc aagatggtat ttctactacc taggaactgg 28621 |
| | | gccagaagct ggacttccct atggtgctaa caaagacggc |
| | | atcatatggg ttgcaactga 28681 gggagccttg aatacaccaa |
| | | aagatcacat tggcacccgc aatcctgcta acaatgctgc 28741 |
| | | aatcgtgcta caacttcctc aaggaacaac attgccaaaa |
| | | ggcttctacg cagaagggag 28801 cagaggcggc |
| | | agtcaagcct cttctogttc ctcatcacgt agtogcaaca |
| | | gttcaagaaa 28861 ttcaactcca ggcagcagta ggggaacttc |
| | | tcctgctaga atggctggca atggcggtga 28921 tgctgctctt |
| | | gctttgctgc tgcttgacag attgaaccag cttgagagca |
| | | aaatgtctgg 28981 taaaggccaa caacaacaag gccaaactgt |
| | | cactaagaaa tctgctgctg aggcttctaa 29041 gaagcctcgg |
| | | caaaaacgta ctgccactaa agcatacaat gtaacacaag |
| | | ctttoggcag 29101 acgtggtcca gaacaaaccc aaggaaattt |
| | | tggggaccag gaactaatca gacaaggaac 29161 tgattacaaa |
| | | cattggccgc aaattgcaca atttgccccc agcgcttcag |
| | | cgttcttcgg 29221 aatgtegcgc attggcatgg aagtcacacc |
| | | ttcgggaacg tggttgacct acacaggtgc 29281 catcaaattg |
| | | gatgacaaag atccaaattt caaagatcaa gtcattttgc |
| | | tgaataagca |
| | | 29341 tattgacgca tacaaaacat toccaccaac agagcctaaa |
| | | aaggacaaaa agaagaaggc 29401 tgatgaaact |
| | | caagccttac cgcagagaca gaagaaacag caaactgtga |
| | | ctcttcttcc 29461 tgctgcagat ttggatgatt tctccaaaca |
| | | attgcaacaa tocatgagca gtgctgactc 29521 aactcaggcc |
| | | taaactcatg cagaccacac aaggcagatg ggctatataa |
| | | acgttttcgc 29581 ttttccgttt acgatatata gtctactctt |
| | | gtgcagaatg aattctcgta actacatagc |
| | | 29641 acaagtagat gtagttaact ttaatctcac atagcaatct |
| | | ttaatcagtg tgtaacatta 29701 gggaggactt gaaagagcca |
| | | ccacattttc accgaggcca cgcggagtac gatcgagtgt |
| | | 29761 acagtgaaca atgctaggga gagctgccta tatggaagag |
| | | ccctaatgtg taaaattaat 29821 tttagtagtg ctatccccat |
| | | gtgattttaa tagcttctta ggagaatgac aaaaaaaaaa 29881 |
| | | aaaaaaaaaa aaaaaaaaaa aaa |
| SARS-CoV-2 | 2 | MESLVPGFNEKTHVQLSLPVLQVRDVLVRGF |
| Wuhan | | GDSVEEVLSEARQHLKDGTCGLVEVEKGVLP |
| seafood | | QLEQPYVFIKRSDARTAPHGHVMVELVAELE |
| market | | GIQYGRSGETLGVLVPHVGEIPVAYRKVLLR |
| pneumonia | | KNGNKGAGGHSYGADLKSFDLGDELGTDPY |
| virus isolate | | EDFQEN |
| Wuhan-Hu-1 | | WNTKHSSGVTRELMRELNGGAYTRYVDNNF |
| genomic | | CGPDGYPLECIKDLLARAGKASCTLSEQLDFI |
| sequence | | DTKRGVYCCREHEHEIAWYTERSEKSYELQT |
| (GenBank: | | PFEIKLAKKFDTFNGECPNFVFPLNSIIKTIQPR |
| MN908947.3; | | VEKKKLDGFMGRIRSVYPVASPNECNQMCLS |
| Jan. 23, 2020)- | | TLMKCDHCGETSWQTGDFVKATCEFCGTEN |
| amino acid | | LTKEGATTCGYLPQNAVVKIYCPACHNSEVG |
| translation | | PEHSLAEYHNESGLKTILRKGGRTIAFGGCVF |
| | | SYVGCHNKCAYWVPRASANIGCNHTGVVGE |
| | | GSEGLNDNL |
| | | LEILQKEKVNINIVGDFKLNEEIAIILASFSAST |
| | | SAFVETVKGLDYKAFKQIVESCGNFKVTKGK |
| | | AKKGAWNIGEQKSILSPLYAFASEAARVVRSI |
| | | FSRTLETAQNSVRVLQKAAITILDGISQYSLRL |
| | | IDAMMFTSDLATNNLVVMAYITGGVVQLTSQ |
| | | WLTNIFGTVYEKLKPVLDWLEEKFKEGVEFL |
| | | RDGWEIVKFISTCACEIVGGQIVTCAKEIKESV |
| | | QTFFKLVNKFLALCADSIIIGGAKLKALNLGE |
| | | TFVTHSKGLYRKCVKSREETGLLMPLKAPKEI |
| | | IFLEGETLPTEVLTEEVVLKTGDLQPLEQPTSE |
| | | AVEAPLVGTPVCINGLMLLEIKDTEKYCALAP |
| | | NMMVTNNTFTLKGGAPTKVTFGDDTVIEVQ |
| | | GYKSVNITFELDERIDKVLNEKCSAYTVELGT |
| | | EVNEFACVVADAVIKTLQPVSELLTPLGIDLD |
| | | EWSMATYYLFDESGEFKLASHMYCSFYPPDE |
| | | DEEEGDCEEEEFEPSTQYEYGTEDDYQGKPLE |
| | | FGATSAALQPEEEQEEDWLDDDSQQTVGQQ |
| | | DGSEDNQTTTIQTIVEVQPQLEMELTPVVQTI |
| | | EVNSFSGYLKLTDNVYIKNADIVEEAKKVKP |
| | | TVVVNAANVYLKHGGGVAGALNKATNNAM |
| | | QVESDDYIATNGPLKVGGSCVLSGHNLAKHC |
| | | LHVVGPNVNKGEDIQLLKSAYENFNQHEVLL |
| | | APLLSAGIFGADPIHSLRVCVDTVRTNVYLAV |
| | | FDKNLYDKLVSSFLEMKSEKQVEQKIAEIPKE |
| | | EVKPFITESKPSVEQRKQDDKKIKACVEEVTT |
| | | TLEETKFLTENLLLYIDINGNLHPDSATLVSDI |
| | | DITFLKKDAPYIVGDVVQEGVLTAVVIPTKKA |
| | | GGTTEMLAKALRKVPTDNYITTYPGQGLNGY |
| | | TVEEAKTVLKKCKSAFYILPSIISNEKQEILGT |
| | | VSWNLREMLAHAEETRKLMPVCVETKAIVST |
| | | IQRKYKGIKIQEGVVDYGARFYFYTSKTTVAS |
| | | LINTLNDLNETLVTMPLGYVTHGLNLEEAAR |
| | | YMRSLKVPATVSVSSPDAVTAYNGYLTSSSK |
| | | TPEEHFIETISLAGSYKDWSYSGQSTQLGIEFL |
| | | KRGDKSVYYTSNPTTFHLDGEVITFDNLKTLL |
| | | SLREVRTIKVFTTVDNINLHTQVVDMSMTYG |
| | | QQFGPTYLDGADVTKIKPHNSHEGKTFYVLP |
| | | NDDTLRVEAFEYYHTTDPSFLGRYMSALNHT |
| | | KKWKYPQVNGLTSIKWADNNCYLATALLTL |
| | | QQIELKFNPPALQDAYYRARAGEAANFCALIL |
| | | AYCNKTVGELGDVRETMSYLFQHANLDSCK |
| | | RVLNVVCKTCGQQQTTLKGVEAVMYMGTLS |
| | | YEQFKKGVQIPCTCGKQATKYLVQQESPFVM |
| | | MSAPPAQYELKHGTFTCASEYTGNYQCGHY |
| | | KHITSKETLYCIDGALLTKSSEYKGPITDVFYK |
| | | ENSYTTTIKPVTYKLDGVVCTEIDPKLDNYYK |
| | | KDNSYFTEQPIDLVPNQPYPNASFDNFKFVCD |
| | | NIKFADDLNQLTGYKKPASRELKVTFFPDLN |
| | | GDVVAIDYKHYTPSFKKGAKLLHKPIVWHVN |
| | | NATNKATYKPNTWCIRCLWSTKPVETSNSFD |
| | | VLKSEDAQGMDNLACEDLKPVSEEVVENPTI |
| | | QKDVLECNVKTTEVVGDIILKPANNSLKITEE |
| | | VGHTDLMAAYVDNSSLTIKKPNELSRVLGLK |
| | | TLATHGLAAVNSVPWDTIANYAKPFLNKVVS |
| | | TTTNIVTRCLNRVCTNYMPYFFTLLLQLCTFT |
| | | RSTNSRIKASMPTTIAKNTVKSVGKFCLEASF |
| | | NYLKSPNFSKLINIIIWFLLLSVCLGSLIYSTAA |
| | | LGVLMSNLGMPSYCTGYREGYLNSTNVTIAT |
| | | YCTGSIPCSVCLSGLDSLDTYPSLETIQITISSF |
| | | KWDLTAFGLVAEWFLAYILFTRFFYVLGLAA |
| | | IMQLFFSYFAVHFISNSWLMWLIINLVQMAPI |
| | | SAMVRMYIFFASFYYVWKSYVHVVDGCNSS |
| | | TCMMCYKRNRATRVECTTIVNGVRRSFYVY |
| | | ANGGKGFCKLHNWNCVNCDTFCAGSTFISDE |
| | | VARDLSLQFKRPINPTDQSSYIVDSVTVKNGSI |
| | | HLYFDKAGQKTYERHSLSHFVNLDNLRANNT |
| | | KGSLPINVIVFDGKSKCEESSAKSASVYYSQL |
| | | MCQPILLLDQALVSDVGDSAEVAVKMFDAY |
| | | VNTFSSTFNVPMEKLKTLVATAEAELAKNVS |
| | | LDNVLSTFISAARQGFVDSDVETKDVVECLK |
| | | LSHQSDIEVTGDSCNNYMLTYNKVENMTPRD |
| | | LGACIDCSARHINAQVAKSHNIALIWNVKDF |
| | | MSLSEQLRKQIRSAAKKNNLPFKLTCATTRQ |
| | | VVNVVTTKIALKGGKIVNNWLKQLIKVTLVF |
| | | LFVAAIFYLITPVHVMSKHTDFSSEIIGYKAID |
| | | GGVTRDIASTDTCFANKHADFDTWFSQRGGS |
| | | YTNDKACPLIAAVITREVGFVVPGLPGTILRTT |
| | | NGDFLHFLPRVFSAVGNICYTPSKLIEYTDFAT |
| | | SACVLAAECTIFKDASGKPVPYCYDTNVLEG |
| | | SVAYESLRPDTRYVLMDGSIIQFPNTYLEGSV |
| | | RVVTTFDSEYCRHGTCERSEAGVCVSTSGRW |
| | | VLNNDYYRSLPGVFCGVDAVNLLTNMFTPLI |
| | | QPIGALDISASIVAGGIVAIVVTCLAYYFMRFR |
| | | RAFGEYSHVVAFNTLLFLMSFTVLCLTPVYSF |
| | | LPGVYSVIYLYLTFYLTNDVSFLAHIQWMVM |
| | | FTPLVPFWITIAYIICISTKHFYWFFSNYLKRRV |
| | | VFNGVSFSTFEEAALCTFLLNKEMYLKLRSD |
| | | VLLPLTQYNRYLALYNKYKYFSGAMDTTSY |
| | | REAACCHLAKALNDFSNSGSDVLYQPPQTSIT |
| | | SAVLQSGFRKMAFPSGKVEGCMVQVTCGTT |
| | | TLNGLWLDDVVYCPRHVICTSEDMLNPNYED |
| | | LLIRKSNHNFLVQAGNVQLRVIGHSMQNCVL |
| | | KLKVDTANPKTPKYKFVRIQPGQTFSVLACY |
| | | NGSPSGVYQCAMRPNFTIKGSFLNGSCGSVGF |
| | | NIDYDCVSFCYMHHMELPTGVHAGTDLEGN |
| | | FYGPFVDRQTAQAAGTDTTITVNVLAWLYA |
| | | AVINGDRWFLNRFTTTLNDFNLVAMKYNYEP |
| | | LTQDHVDILGPLSAQTGIAVLDMCASLKELLQ |
| | | NGMNGRTILGSALLEDEFTPFDVVRQCSGVTF |
| | | QSAVKRTIKGTHHWLLLTILTSLLVLVQSTQ |
| | | WSLFFFLYENAFLPFAMGIIAMSAFAMMFVK |
| | | HKHAFLCLFLLPSLATVAYFNMVYMPASWV |
| | | MRIMTWLDMVDTSLSGFKLKDCVMYASAVV |
| | | LLILMTARTVYDDGARRVWTLMNVLTLVYK |
| | | VYYGNALDQAISMWALIISVTSNYSGVVTTV |
| | | MFLARGIVFMCVEYCPIFFITGNTLQCIMLVY |
| | | CFLGYFCTCYFGLFCLLNRYFRLTLGVYDYL |
| | | VSTQEFRYMNSQGLLPPKNSIDAFKLNIKLLG |
| | | VGGKPCIKVATVQSKMSDVKCTSVVLLSVLQ |
| | | QLRVESSSKLWAQCVQLHNDILLAKDTTEAF |
| | | EKMVSLLSVLLSMQGAVDINKLCEEMLDNR |
| | | ATLQAIASEFSSLPSYAAFATAQEAYEQAVAN |
| | | GDSEVVLKKLKKSLNVAKSEFDRDAAMQRK |
| | | LEKMADQAMTQMYKQARSEDKRAKVTSAM |
| | | QTMLFTMLRKLDNDALNNIINNARDGCVPLN |
| | | IIPLTTAAKLMVVIPDYNTYKNTCDGTTFTYA |
| | | SALWEIQQVVDADSKIVQLSEISMDNSPNLA |
| | | WPLIVTALRANSAVKLQNNELSPVALRQMSC |
| | | AAGTTQTACTDDNALAYYNTTKGGRFVLAL |
| | | LSDLQDLKWARFPKSDGTGTIYTELEPPCRFV |
| | | TDTPKGPKVKYLYFIKGLNNLNRGMVLGSLA |
| | | ATVRLQAGNATEVPANSTVLSFCAFAVDAAK |
| | | AYKDYLASGGQPITNCVKMLCTHTGTGQAIT |
| | | VTPEANMDQESFGGASCCLYCRCHIDHPNPK |
| | | GFCDLKGKYVQIPTTCANDPVGFTLKNTVCT |
| | | VCGMWKGYGCSCDQLREPMLQSADAQSFLN |
| | | RVCGVSAARLTPCGTGTSTDVVYRAFDIYND |
| | | KVAGFAKFLKTNCCRFQEKDEDDNLIDSYFV |
| | | VKRHTFSNYQHEETIYNLLKDCPAVAKHDFF |
| | | KFRIDGDMVPHISRQRLTKYTMADLVYALRH |
| | | FDEGNCDTLKEILVTYNCCDDDYFNKKDWY |
| | | DFVENPDILRVYANLGERVRQALLKTVQFCD |
| | | AMRNAGIVGVLTLDNQDLNGNWYDFGDFIQ |
| | | TTPGSGVPVV |
| | | DSYYSLLMPILTLTRALTAESHVDTDLTKPYI |
| | | KWDLLKYDFTEERLKLFDRYFKYWDQTYHP |
| | | NCVNCLDDRCILHCANFNVLFSTVFPPTSFGP |
| | | LVRKIFVDGVPFVVSTGYHFRELGVVHNQDV |
| | | NLHSSRLSFKELLVYAADPAMHAASGNLLLD |
| | | KRTTCFSVAALTNNVAFQTVKPGNFNKDFYD |
| | | FAVSKGFFKEGSSVELKHFFFAQDGNAAISDY |
| | | DYYRYNLPTMCDIRQLLFVVEVVDKYFDCY |
| | | DGGCINANQVIVNNLDKSAGFPFNKWGKARL |
| | | YYDSMSYEDQDALFAYTKRNVIPTITQMNLK |
| | | YAISAKNRARTVAGVSICSTMTNRQFHQKLL |
| | | KSIAATRGATVVIGTSKFYGGWHNMLKTVYS |
| | | DVENPHLMGWDYPKCDRAMPNMLRIMASL |
| | | VLARKHTTCCSLSHRFYRLANECAQVLSEMV |
| | | MCGGSLYVKPGGTSSGDATTAYANSVFNICQ |
| | | AVTANVNALLSTDGNKIADKYVRNLQHRLY |
| | | ECLYRNRDVDTDFVNEFYAYLRKHFSMMILS |
| | | DDAVVCFNSTYASQGLVASIKNFKSVLYYQN |
| | | NVFMSEAKCWTETDLTKGPHEFCSQHTMLV |
| | | KQGDDYVYLPYPDPSRILGAGCFVDDIVKTD |
| | | GTLMIERFVSLAIDAYPLTKHPNQEYADVFHL |
| | | YLQYIRKLHDELTGHMLDMYSVMLINDNTS |
| | | RYWEPEFYEAMYTPHTVLQAVGACVLCNSQ |
| | | TSLRCGACIRRPFLCCKCCYDHVISTSHKLVL |
| | | SVNPYVCNAPGCDVTDVTQLYLGGMSYYCK |
| | | SHKPPISFPLCANGQVFGLYKNTCVGSDNVTD |
| | | FNAIATCDWTNAGDYILANTCTERLKLFAAE |
| | | TLKATEETFKLSYGIATVREVLSDRELHLSWE |
| | | VGKPRPPLNRNYVFTGYRVTKNSKVQIGEYT |
| | | FEKGDYGDAVVYRGTTTYKLNVGDYFVLTS |
| | | HTVMPLSAPTLVPQEHYVRITGLYPTLNISDE |
| | | FSSNVANYQKVGMQKYSTLQGPPGTGKSHF |
| | | AIGLALYYPSARIVYTACSHAAVDALCEKAL |
| | | KYLPIDKCSRIIPARARVECFDKFKVNSTLEQY |
| | | VFCTVNALPETTADIVVFDEISMATNYDLSVV |
| | | NARLRAKHYVYIGDPAQLPAPRTLLTKGTLE |
| | | PEYFNSVCRLMKTIGPDMFLGTCRRCPAEIVD |
| | | TVSALVYDNKLKAHKDKSAQCFKMFYKGVI |
| | | THDVSSAINRPQIGVVREFLTRNPAWRKAVFI |
| | | SPYNSQNAVASKILGLPTQTVDSSQGSEYDYV |
| | | IFTQTTETAHSCNVNRFNVAITRAKVGILCIMS |
| | | DRDLYDKLQFTSLEIPRRNVATLQAENVTGLF |
| | | KDCSKVITGLHPTQAPTHLSVDTKFKTEGLCV |
| | | DIPGIPKDMTYRRLISMMGFKMNYQVNGYPN |
| | | MFITREEAIRHVRAWIGFDVEGCHATREAVG |
| | | TNLPLQLGFSTGVNLVAVPTGYVDTPNNTDF |
| | | SRVSAKPPPGDQFKHLIPLMYKGLPWNVVRI |
| | | KIVQMLSDTLKNLSDRVVFVLWAHGFELTSM |
| | | KYFVKIGPERTCCLCDRRATCFSTASDTYAC |
| | | WHHSIGFDYVYNPFMIDVQQWGFTGNLQSN |
| | | HDLYCQVHGNAHVASCDAIMTRCLAVHECF |
| | | VKRVDWTIEYPIIGDELKINAACRKVQHMVV |
| | | KAALLADKFPVLHDIGNPKAIKCVPQADVEW |
| | | KFYDAQPCSDKAYKIEELFYSYATHSDKFTD |
| | | GVCLFWNCNVDRYPANSIVCRFDTRVLSNLN |
| | | LPGCDGGSLYVNKHAFHTPAFDKSAFVNLKQ |
| | | LPFFYYSDSPCESHGKQVVSDIDYVPLKSATCI |
| | | TRCNLGGAVCRHHANEYRLYLDAYNMMISA |
| | | GFSLWVYKQFDTYNLWNTFTRLQSLENVAF |
| | | NVVNKGHFDGQQGEVPVSIINNTVYTKVDGV |
| | | DVELFENKTTLPVNVAFELWAKRNIKPVPEV |
| | | KILNNLGVDIAANTVIWDYKRDAPAHISTIGV |
| | | CSMTDIAKKPTETICAPLTVFFDGRVDGQVDL |
| | | FRNARNGVLITEGSVKGLQPSVGPKQASLNG |
| | | VTLIGEAVKTQFNYYKKVDGVVQQLPETYFT |
| | | QSRNLQEFKPRSQMEIDFLELAMDEFIERYKL |
| | | EGYAFEHIVYGDFSHSQLGGLHLLIGLAKRFK |
| | | ESPFELEDFIPMDSTVKNYFITDAQTGSSKCVC |
| | | SVIDLLLDDFVEIIKSQDLSVVSKVVKVTIDYT |
| | | EISFMLWCKDGHVETFYPKLQSSQAWQPGVA |
| | | MPNLYKMQRMLLEKCDLQNYGDSATLPKGI |
| | | MMNVAKYTQLCQYLNTLTLAVPYNMRVIHF |
| | | GAGSDKGVAPGTAVLRQWLPTGTLLVDSDL |
| | | NDFVSDADSTLIGDCATVHTANKWDLIISDM |
| | | YDPKTKNVTKENDSKEGFFTYICGFIQQKLAL |
| | | GGSVAIKITEHSWNADLYKLMGHFAWWTAF |
| | | VTNVNASSSEAFLIGCNYLGKPREQIDGYVM |
| | | HANYIFWRNTNPIQLSSYSLFDMSKFPLKLRG |
| | | TAVMSLKEGQINDMILSLLSKGRLIIRENNRV |
| | | VISSDVLVNN |
| surface | 3 | mfvflvllpl vssqcvnltt rtqlppaytn sftrgvyypd |
| glycoprotein | | kvfrssvlhs tqdlflpffs 61 nvtwfhaihv sgtngtkrfd |
| [SARS-CoV- | | npvlpfndgv yfasteksni irgwifgttl dsktqslliv 121 |
| 2 Wuhan | | nnatnvvikv cefqfcndpf lgvyyhknnk swmesefrvy |
| seafood | | ssannctfey vsqpflmdle181 gkqgnfknlr efvfknidgy |
| market | | fkiyskhtpi nlvrdlpqgf saleplvdlp iginitrfqt 241 |
| pneumonia | | llalhrsylt pgdsssgwta gaaayyvgyl qprtfllkyn |
| virus]; | | engtitdavd caldplsetk 301 ctlksftvek giyqtsnfrv |
| GenBank: | | qptesivrfp nitnlcpfge vfnatrfasv yawnrkrisn 361 |
| QHD43416.1; | | cvadysvlyn sasfstfkcy gvsptklndl cftnvyadsf |
| Jan. 23, 2020 | | virgdevrqi apgqtgkiad 421 ynyklpddft gcviawnsnn |
| | | ldskvggnyn ylyrlfrksn lkpferdist eiyqagstpc 481 |
| | | ngvegfncyf plqsygfqpt ngvgyqpyrv vvlsfellha |
| | | patvcgpkks tnlvknkcvn 541 fnfngltgtg vltesnkkfl |
| | | pfqqfgrdia dttdavrdpq tleilditpc sfggvsvitp 601 |
| | | gtntsnqvav lyqdvnctev pvaihadqlt ptwrvystgs |
| | | nvfqtragcl igaehvnnsy 661 ecdipigagi casyqtqtns |
| | | prrarsvasq siiaytmslg aensvaysnn siaiptnfti 721 |
| | | svtteilpvs mtktsvdctm yicgdstecs nlllqygsfc |
| | | tqlnraltgi aveqdkntqe 781 vfaqvkqiyk tppikdfggf |
| | | nfsqilpdps kpskrsfied llfnkvtlad agfikqygdc 841 |
| | | lgdiaardli caqkfngltv lpplltdemi aqytsallag |
| | | titsgwtfga gaalqipfam 901 qmayrfngig vtqnvlyenq |
| | | klianqfnsa igkiqdslss tasalgklqd vvnqnaqaln 961 |
| | | tlvkqlssnf gaissvlndi lsrldkveae vqidrlitgr |
| | | lqslqtyvtq qliraaeira |
| | | 1021 sanlaatkms ecvlgqskrv dfcgkgyhlm |
| | | sfpqsaphgv vflhvtyvpa qeknfttapa 1081 ichdgkahfp |
| | | regvfvsngt hwfvtqrnfy epqiittdnt fvsgncdvvi |
| | | givnntvydp 1141 lqpeldsfke eldkyfknht spdvdlgdis |
| | | ginasvvniq keidrlneva knlneslidl 1201 qelgkyeqyi |
| | | kwpwyiwlgf iagliaivmv timlccmtsc csclkgccsc |
| | | gscckfdedd 1261 sepvlkgvkl hyt |
| surface | 4 | nitnlcpfgevfnatrfasvyawnrkrisncvadysvlynsasfstfkc |
| glycoprotein | | ygvsptklndlcftnvyadsfvirgdevrqiapgqtgkiadynyklpd |
| RBD [SARS- | | dftgcviawnsnnldskvggnynylyrlfrksnlkpferdisteiyqa |
| CoV-2 | | gstpcngvegfncyfplqsygfqptngvgyqpyrvvvlsfellhapa |
| Wuhan | | tvcgpkkstnlvknkcvnfnfngltgtg |
| seafood | | |
| market | | |
| pneumonia | | |
| virus]; | | |
| GenBank: | | |
| QHD43416.1; | | |
| Jan. 23, 2020 | | |
| Receptor | 5 | Nsnnldskvggnynylyrlfrksnlkpferdisteiyqagstpcngve |
| Binding Motif | | gfncyfplqsygfqptngvgyqpy |
| (RBM) in | | |
| surface | | |
| glycoprotein | | |
| RBD [SARS- | | |
| CoV-2 | | |
| Wuhan | | |
| seafood | | |
| market | | |
| pneumonia | | |
| virus]; | | |
| GenBank: | | |
| QHD43416.1; | | |
| Jan. 23, 2020 | | |
| SARS-CoV-2 | 6 | ASTKGPSVFPLAPSSKSTSGGTAALGCLVKDY |
| CH1-CH3 LS | | FPEPVTVSWNSGALTSGVHTFPAVLQSSGLYS |
| G1m17 | | LSSVVTVPSSSLGTQTYICNVNHKPSNTKVDK |
| IgHG1*01 | | KVEPKSCDKTHTCPPCPAPELLGGPSVFLFPP |
| (aa) | | KPKDTLMISRTPEVTCVVVDVSHEDPEVKFN |
| | | WYVDGVEVHNAKTKPREEQYNSTYRVVSVL |
| | | TVLHQDWLNGKEYKCKVSNKALPAPIEKTIS |
| | | KAKGQPREPQVYTLPPSRDELTKNQVSLTCL |
| | | VKGFYPSDIAVEWESNGQPENNYKTTPPVLD |
| | | SDGSFFLYSKLTVDKSRWQQGNVFSCSVLHE |
| | | ALHSHYTQKSLSLSPGK |
| SARS-CoV-2 | 7 | ASTKGPSVFPLAPSSKSTSGGTAALGCLVKDY |
| CH1-CH3 LS, | | FPEPVTVSWNSGALTSGVHTFPAVLQSSGLYS |
| ALE G1m17 | | LSSVVTVPSSSLGTQTYICNVNHKPSNTKVDK |
| IgHG1*01 | | KVEPKSCDKTHTCPPCPAPELLAGPSVFLFPP |
| (aa) | | KPKDTLMISRTPEVTCVVVDVSHEDPEVKFN |
| | | WYVDGVEVHNAKTKPREEQYNSTYRVVSVL |
| | | TVLHQDWLNGKEYKCKVSNKALPLPEEKTIS |
| | | KAKGQPREPQVYTLPPSRDELTKNQVSLTCL |
| | | VKGFYPSDIAVEWESNGQPENNYKTTPPVLD |
| | | SDGSFFLYSKLTVDKSRWQQGNVFSCSVLHE |
| | | ALHSHYTQKSLSLSPGK |
| SARS-CoV-2 | 8 | GQPKAAPSVTLFPPSSEELQANKATLVCLISDF |
| CL IgLC*01 | | YPGAVTVAWKADSSPVKAGVETTTPSKQSN |
| (aa) | | NKYAASSYLSLTPEQWKSHRSYSCQVTHEGS |
| | | TVEKTVAPTECS |
| SARS-CoV-2 | 9 | RTVAAPSVFIFPPSDEQLKSGTASVVCLLNNF |
| CL (CK) | | YPREAKVQWKVDNALQSGNSQESVTEQDSK |
| k1m3 | | DSTYSLSSTLTLSKADYEKHKVYACEVTHQG |
| IgKC*01 (aa) | | LSSPVTKSFNRGEC |
| Linker (aa) | 10 | GSTSGSGKPGSGEGSTKG |
| Linker (aa) | 11 | GSGKPGSGEG |
| Linker (aa) | 12 | GKPGSGEG |
| Linker (aa) | 13 | SGKPGSGE |
| Linker (aa) | 14 | BPXXXZ, wherein each X is independently a |
| | | glycine (G) or serine (S), B is a positively charged |
| | | amino acid and Z is glycine (G) or a negatively |
| | | charged amino acid |
| Linker (aa) | 15 | (GxS)y, wherein x is 1-10 and y is 1-10 |
| Linker (aa) | 16 | GGGGSGGGGSGGGGS |
| Linker (aa) | 17 | GGGGSGGGGSGGGGSGGGGSGGGGS |
| | | GGGGSGGGGSGGGGSGGGGSGGGGS |
| Linker (aa) | 18 | GSTSGGGSGGGSGGGGSS |
| Linker (aa) | 19 | EGKSSGSGSESKVD |
| Linker (aa) | 20 | KESGSVSSEQLAQFRSLD |
| Linker (aa) | 21 | GGGGS |
| SARS-CoV-2 | 22 | EVQLVESGGGLVQPGGSLRLSCAASGFIVSSN |
| S2H7-v1 | | YMSWVRQAPGKGLEWVSVIYSGGSTYYADS |
| mAb VH (aa) | | VKGRFTISRDNSKNTLYLQMNSLRAEDTSVY |
| | | YCARDRQYSGSPSFDYWGQGTLVTVSS |
| SARS-CoV-2 | 23 | GFIVSSNY |
| S2H7-v1 | | |
| mAb CDRH1 | | |
| (aa) | | |
| SARS-CoV-2 | 24 | IYSGGST |
| S2H7-v1 | | |
| mAb CDRH2 | | |
| (aa) | | |
| SARS-CoV-2 | 25 | ARDRQYSGSPSFDY |
| S2H7-v1 | | |
| mAb CDRH3 | | |
| (aa) | | |
| SARS-CoV-2 | 26 | DIQMTQSPSSLSASVGDRVTITCRASQSISSYL |
| S2H7-v1 | | NWYQQKPGKAPKLLIYTASSLQSGVPSRFSGS |
| mAb VL(VK) | | GSGTDFTLTISSLQPEDFATYYCQQSYSTPGL |
| (aa) | | TFGGGTKVEIK |
| SARS-CoV-2 | 27 | QSISSY |
| S2H7-v1 | | |
| mAb CDRL1 | | |
| (aa) | | |
| SARS-CoV-2 | 28 | TAS |
| S2H7-v1 | | |
| mAb CDRL2 | | |
| (aa) | | |
| SARS-CoV-2 | 29 | QQSYSTPGLT |
| S2H7-v1 | | |
| mAb CDRL3 | | |
| (aa) | | |
| SARS-CoV-2 | 30 | EVQLVESGGGLVQPGGSLRLSCAASGFIVSSN |
| S2H7-v2 | | YMSWVRQAPGKGLEWVSVIYSGGSTYYAES |
| mAb VH (aa) | | VKGRFTISRDNSKNTLYLQMNSLRAEDTSVY |
| | | YCARDRQYSGSPSFDYWGQGTLVTVSS |
| SARS-CoV-2 | 31 | VIYSGGSTYYAESVKG |
| S2H7-v2 | | |
| mAb CDRH2 | | |
| (aa) | | |
| SARS-CoV-2 | 32 | EVQLVESGGGLVQPGGSLRLSCAASGFIVSSN |
| S2H7-v3 | | YISWVRQAPGKGLEWVSVIYSGGSTYYADS |
| mAb VH (aa) | | VKGRFTISRDNSKNTLYLQMNSLRAEDTSVY |
| | | YCARDRQYSGSPSFDYWGQGTLVTVSS |
| SARS-CoV-2 | 33 | GFIVSSNYIS |
| S2H7-v3 | | |
| mAb CDRH1 | | |
| (aa) | | |
| SARS-CoV-2 | 34 | EVQLVESGGGLVQPGGSLRLSCAASGFIVSSN |
| S2H7-v4 | | YISWVRQAPGKGLEWVSVIYSGGSTYYAES |
| mAb VH (aa) | | VKGRFTISRDNSKNTLYLQMNSLRAEDTSVY |
| | | YCARDRQYSGSPSFDYWGQGTLVTVSS |
| SARS-CoV-2 | 35 | EVQLVESGGGLVQPGGSLRLSCAASGFIVSSN |
| S2H7-v5 | | YISWVRQAPGKGLEWVSVIYSGGSTYYADA |
| mAb VH (aa) | | VKGRFTISRDNSKNTLYLQMNSLRAEDTSVY |
| | | YCARDRQYSGSPSFDYWGQGTLVTVSS |
| SARS-CoV-2 | 36 | VIYSGGSTYYADAVKG |
| S2H7-v5 | | |
| mAb CDRH2 | | |
| (aa) | | |
| SARS-CoV-2 | 37 | EVQLVESGGDSVQPGGSLRLSCAAAGFTFSS |
| S2H13-v1 | | YWMNWVRQAPGKGLEWVANIKQDGSEKY |
| mAb VH (aa) | | YVDSVKGRFTISRDNAKNSLYLQMNSLRAED |
| | | TAVYYCALSSGYSGYAGNYWGQGTLVTVSS |
| SARS-CoV-2 | 38 | GFTFSSYW |
| S2H13-v1 | | |
| mAb CDRH1 | | |
| (aa) | | |
| SARS-CoV-2 | 39 | IKQDGSEK |
| S2H13-v1 | | |
| mAb CDRH2 | | |
| (aa) | | |
| SARS-CoV-2 | 40 | ALSSGYSGYAGNY |
| S2H13-v1 | | |
| mAb CDRH3 | | |
| (aa) | | |
| SARS-CoV-2 | 41 | QAVVTQEPSLTVSPGGTVTLTCGSSTGAVTS |
| S2H13-v1 | | GHYPYWFQQKPGQAPRTLIYDTSNKHSWTP |
| mAb VL (aa) | | ARFSGSLLGGKAALTLSGARPEDEAEYYCLL |
| | | SYSGARGVFGGGTKLTVL |
| SARS-CoV-2 | 42 | TGAVTSGHY |
| S2H13-v1 | | |
| mAb CDRL1 | | |
| (aa) | | |
| SARS-CoV-2 | 43 | DTS |
| S2H13-v1 | | |
| mAb CDRL2 | | |
| (aa) | | |
| SARS-CoV-2 | 44 | LLSYSGARGV |
| S2H13-v1 | | |
| mAb CDRL3 | | |
| (aa) | | |
| SARS-CoV-2 | 45 | EVQLVESGGDSVQPGGSLRLSCAAAGFTFSS |
| S2H13-v2 | | YFMNWVRQAPGKGLEWVANIKQDGSEKYY |
| mAb VH (aa) | | VDSVKGRFTISRDNAKNSLYLQMNSLRAEDT |
| | | AVYYCALSSGYSGYAGNYWGQGTLVTVSS |
| SARS-CoV-2 | 46 | GFTFSSYFMN |
| S2H13-v2 | | |
| mAb CDRH1 | | |
| (aa) | | |
| SARS-CoV-2 | 47 | EVQLVESGGDSVQPGGSLRLSCAAAGFTFSS |
| S2H13-v3 | | YWMNWVRQAPGKGLEWVANIKQEGSEKY |
| mAb VH (aa) | | YVDSVKGRFTISRDNAKNSLYLQMNSLRAED |
| | | TAVYYCALSSGYSGYAGNYWGQGTLVTVSS |
| SARS-CoV-2 | 48 | NIKQEGSEKYYVDSVKG |
| S2H13-v3 | | |
| mAb CDRH2 | | |
| (aa) | | |
| SARS-CoV-2 | 49 | EVQLVESGGDSVQPGGSLRLSCAAAGFTFSS |
| S2H13-v4 | | YFMNWVRQAPGKGLEWVANIKQEGSEKYY |
| mAb VH (aa) | | VDSVKGRFTISRDNAKNSLYLQMNSLRAEDT |
| | | AVYYCALSSGYSGYAGNYWGQGTLVTVSS |
| SARS-CoV-2 | 50 | EVQLVESGGDSVQPGGSLRLSCAAAGFTFSS |
| S2H13-v5 | | YFMNWVRQAPGKGLEWVANIKQDASEKYY |
| mAb VH (aa) | | VDSVKGRFTISRDNAKNSLYLQMNSLRAEDT |
| | | AVYYCALSSGYSGYAGNYWGQGTLVTVSS |
| SARS-CoV-2 | 51 | NIKQDASEKYYVDSVKG |
| S2H13-v5 | | |
| mAb CDRH2 | | |
| (aa) | | |
| SARS-CoV-2 | 52 | EVQLVESGGDSVQPGGSLRLSCAAAGFTFSS |
| S2H13-v6 | | YYMNWVRQAPGKGLEWVANIKQEGSEKYY |
| mAb VH (aa) | | VDSVKGRFTISRDNAKNSLYLQMNSLRAEDT |
| | | AVYYCALSSGYSGYAGNYWGQGTLVTVSS |
| SARS-CoV-2 | 53 | GFTFSSYYMN |
| S2H13-v6 | | |
| mAb CDRH1 | | |
| (aa) | | |
| SARS-CoV-2 | 54 | EVQLVESGGGLVKPGGSLRLSCAASGFTFSN |
| S2H14-v1 | | AWMSWVRQAPGKGLEWVGRIKSKTDGGTT |
| mAb VH (aa) | | DYAAPVKGRFTISRDDSKNTLYLQMNSLKTE |
| | | DTAVYYCTTGSETYYYDSSGPFDYWGQGTL |
| | | VTVSS |
| SARS-CoV-2 | 55 | GFTFSNAWMS |
| S2H14-v1 | | |
| mAb CDRH1 | | |
| (aa) | | |
| SARS-CoV-2 | 56 | RIKSKTDGGTTDYAAPVKG |
| S2H14-v1 | | |
| mAb CDRH2 | | |
| (aa) | | |
| SARS-CoV-2 | 57 | GSETYYYDSSGPFDY |
| S2H14-v1 | | |
| mAb CDRH3 | | |
| (aa) | | |
| SARS-CoV-2 | 58 | NFMLTQPHSVSESPGKTVTISCTRSSGSIASNY |
| S2H14-v1 | | VQWYQQRPGSSPTTVIYEDNQRPSGVPDRFS |
| mAb VL (aa) | | GSIDSSSNSASLTISGLKTEDEADYYCQSYDSS |
| | | NQVFGGGTKLTVL |
| SARS-CoV-2 | 59 | TRSSGSIASNYVQ |
| S2H14-v1 | | |
| mAb CDRL1 | | |
| (aa) | | |
| SARS-CoV-2 | 60 | EDNQRPS |
| S2H14-v1 | | |
| mAb CDRL2 | | |
| (aa) | | |
| SARS-CoV-2 | 61 | QSYDSSNQV |
| S2H14-v1 | | |
| mAb CDRL3 | | |
| (aa) | | |
| SARS-CoV-2 | 62 | EVQLVESGGGLVKPGGSLRLSCAASGFTFSN |
| S2H14-v2 | | AFMSWVRQAPGKGLEWVGRIKSKTDGGTT |
| mAb VH (aa) | | DYAAPVKGRFTISRDDSKNTLYLQMNSLKTE |
| | | DTAVYYCTTGSETYYYDSSGPFDYWGQGTL |
| | | VTVSS |
| SARS-CoV-2 | 63 | GFTFSNAFMS |
| S2H14-v2 | | |
| mAb CDRH1 | | |
| (aa) | | |
| SARS-CoV-2 | 64 | EVQLVESGGGLVKPGGSLRLSCAASGFTFSN |
| S2H14-v3 | | AWMSWVRQAPGKGLEWVGRIKSKTEGGTT |
| mAb VH (aa) | | DYAAPVKGRFTISRDDSKNTLYLQMNSLKTE |
| | | DTAVYYCTTGSETYYYDSSGPFDYWGQGTL |
| | | VTVSS |
| SARS-CoV-2 | 65 | RIKSKTEGGTTDYAAPVKG |
| S2H14-v3 | | |
| mAb CDRH2 | | |
| (aa) | | |
| SARS-CoV-2 | 66 | EVQLVESGGGLVKPGGSLRLSCAASGFTFSN |
| S2H14-v4 | | AFMSWVRQAPGKGLEWVGRIKSKTDAGTT |
| mAb VH (aa) | | DYAAPVKGRFTISRDDSKNTLYLQMNSLKTE |
| | | DTAVYYCTTGSETYYYDSSGPFDYWGQGTL |
| | | VTVSS |
| SARS-CoV-2 | 67 | RIKSKTDAGTTDYAAPVKG |
| S2H14-v4 | | |
| mAb CDRH2 | | |
| (aa) | | |
| SARS-CoV-2 | 68 | EVQLVESGGGLVKPGGSLRLSCAASGFTFSN |
| S2H14-v5 | | AFMSWVRQAPGKGLEWVGRIKSKTEGGTT |
| mAb VH (aa) | | DYAAPVKGRFTISRDDSKNTLYLQMNSLKTE |
| | | DTAVYYCTTGSETYYYDSSGPFDYWGQGTL |
| | | VTVSS |
| SARS-CoV-2 | 69 | EVQLVESGGGLVKPGGSLRLSCAASGFTFSN |
| S2H14-v6 | | AYMSWVRQAPGKGLEWVGRIKSKTEGGTT |
| mAb VH (aa) | | DYAAPVKGRFTISRDDSKNTLYLQMNSLKTE |
| | | DTAVYYCTTGSETYYYDSSGPFDYWGQGTL |
| | | VTVSS |
| SARS-CoV-2 | 70 | GFTFSNAYMS |
| S2H14-v6 | | |
| mAb CDRH1 | | |
| (aa) | | |
| SARS-CoV-2 | 71 | EVQLVESGGGLVQPGGSLRLSCAASGFTFSS |
| S2A4-v1 | | YWMNWVRQAPGKGLEWVANIKQDGSEKY |
| mAb VH (aa) | | YVDSVKGRFTISRDNAKNSLFLQMNSLRAED |
| | | TAVYYCARVWWLRGSFDYWGQGTLVTVSS |
| SARS-CoV-2 | 72 | GFTFSSYW |
| S2A4-v1 | | |
| mAb CDRH1 | | |
| (aa) | | |
| SARS-CoV-2 | 73 | IKQDGSEK |
| S2A4-v1 | | |
| mAb CDRH2 | | |
| (aa) | | |
| SARS-CoV-2 | 74 | ARVWWLRGSFDY |
| S2A4-v1 | | |
| mAb CDRH3 | | |
| (aa) | | |
| SARS-CoV-2 | 75 | NFMLTQPHSVSESPGKTVTISCTGSSGSIASNY |
| S2A4-v1 | | VQWYQQRPGSAPTTVIYEDNQRPSGVPDRFS |
| mAb VL (aa) | | GSIDSSSNSASLTISGLKTEDEADYYCQSYDSS |
| | | NHVVFGGGTKLTVL |
| SARS-CoV-2 | 76 | SGSIASNY |
| S2A4-v1 | | |
| mAb CDRL1 | | |
| (aa) | | |
| SARS-CoV-2 | 77 | EDN |
| S2A4-v1 | | |
| mAb CDRL2 | | |
| (aa) | | |
| SARS-CoV-2 | 78 | QSYDSSNHVV |
| S2A4-v1 | | |
| mAb CDRL3 | | |
| (aa) | | |
| SARS-CoV-2 | 79 | GAGGTGCAGCTGGTGGAGTCTGGGGGAGG |
| S2A4-v1 | | CTTGGTCCAGCCTGGGGGGTCCCTGAGACT |
| mAb VH (nt) | | CTCCTGTGCAGCCTCTGGATTCACCTTTAG |
| | | TAGCTATTGGATGAACTGGGTCCGCCAGG |
| | | CTCCAGGGAAGGGGCTGGAGTGGGTGGCC |
| | | AACATAAAGCAAGATGGAAGTGAGAAAT |
| | | ACTATGTGGACTCTGTGAAGGGCCGATTCA |
| | | CCATCTCCAGAGACAACGCCAAGAACTCAC |
| | | TGTTTCTGCAAATGAACAGCCTGAGAGCCG |
| | | AGGACACGGCTGTGTATTACTGTGCGAGA |
| | | GTCTGGTGGCTACGAGGTTCCTTTGACT |
| | | ACTGGGGCCAGGGAACCCTGGTCACCGTCT |
| | | CCTCAG |
| SARS-CoV-2 | 80 | AATTTTATGCTGACTCAGCCCCACTCTGTGT |
| S2A4-v1 | | CGGAGTCTCCGGGGAAGACGGTAACCATCT |
| mAb VL (nt) | | CCTGCACCGGCAGCAGTGGCAGCATTGCC |
| | | AGCAACTATGTGCAGTGGTACCAGCAGCG |
| | | CCCGGGCAGTGCCCCCACCACTGTGATCTA |
| | | TGAGGATAACCAAAGACCCTCTGGGGTCC |
| | | CTGATCGGTTCTCTGGCTCCATCGACAGCTC |
| | | CTCCAACTCTGCCTCCCTCACCATCTCTGGA |
| | | CTGAAGACTGAGGACGAGGCTGACTACTAC |
| | | TGTCAGTCTTATGATAGCAGCAATCATGT |
| | | GGTATTCGGCGGAGGGACCAAGCTGACCG |
| | | TCCTAG |
| SARS-CoV-2 | 81 | EVQLVESGGGLVQPGRSLRLSCAASGFTFDD |
| S2A5-v1 | | YAMHWVRQAPGKGLRWVSGISWNSGSIGY |
| mAb VH (aa) | | ADSVKGRFTISRDNAKNSLYLQMNSLRAEDT |
| | | ALYYCAKEVGKNYYDSSGYERDYFDYWGQ |
| | | GTLVTVSS |
| SARS-CoV-2 | 82 | GFTFDDYA |
| S2A5-v1 | | |
| mAb CDRH1 | | |
| (aa) | | |
| SARS-CoV-2 | 83 | ISWNSGSI |
| S2A5-v1 | | |
| mAb CDRH2 | | |
| (aa) | | |
| SARS-CoV-2 | 84 | AKEVGKNYYDSSGYERDYFDY |
| S2A5-v1 | | |
| mAb CDRH3 | | |
| (aa) | | |
| SARS-CoV-2 | 85 | SYVLTQPPSVSVAPGKTARITCGGNNIGSKSV |
| S2A5-v1 | | HWYQQKPGQAPVLVIYYDSDRPSGIPERFSGS |
| mAb VL (aa) | | NSGNTATLTISRVEAGDEADYYCQVWDSSSD |
| | | HYVFGTGTKVTVL |
| SARS-CoV-2 | 86 | NIGSKS |
| S2A5-v1 | | |
| mAb CDRL1 | | |
| (aa) | | |
| SARS-CoV-2 | 87 | YDS |
| S2A5-v1 | | |
| mAb CDRL2 | | |
| (aa) | | |
| SARS-CoV-2 | 88 | QVWDSSSDHYV |
| S2A5-v1 | | |
| mAb CDRL3 | | |
| (aa) | | |
| SARS-CoV-2 | 89 | GAAGTGCAGCTGGTGGAGTCTGGGGGAGG |
| S2A5-v1 | | CTTGGTACAGCCTGGCAGGTCCCTGAGACT |
| mAb VH (nt) | | CTCCTGTGCAGCCTCTGGATTCACCTTTGA |
| | | TGATTATGCCATGCACTGGGTCCGGCAAG |
| | | CTCCAGGGAAGGGCCTGCGGTGGGTCTCAG |
| | | GTATTAGTTGGAATAGTGGTAGCATAGGC |
| | | TATGCGGACTCTGTGAAGGGCCGATTCACC |
| | | ATCTCCAGAGACAACGCCAAGAACTCCCTG |
| | | TATCTGCAAATGAACAGTCTGAGAGCTGAG |
| | | GACACGGCCTTGTATTACTGTGCAAAAGAG |
| | | GTGGGAAAGAATTACTATGATAGTAGTG |
| | | GTTATGAAAGGGACTACTTTGACTACTGG |
| | | GGCCAGGGAACCCTGGTCACCGTCTCCTCA |
| | | G |
| SARS-CoV-2 | 90 | TCCTATGTGCTGACTCAGCCACCCTCAGTGT |
| S2A5-v1 | | CAGTGGCCCCAGGAAAGACGGCCAGGATT |
| mAb VL (nt) | | ACCTGTGGGGGAAACAACATTGGAAGTAA |
| | | AAGTGTGCACTGGTACCAGCAGAAGCCAG |
| | | GCCAGGCCCCTGTGCTGGTCATCTATTATG |
| | | ATAGCGACCGGCCCTCAGGGATCCCTGAGC |
| | | GATTCTCTGGCTCCAACTCTGGGAACACGG |
| | | CCACCCTGACCATCAGCAGGGTCGAAGCCG |
| | | GGGATGAGGCCGACTATTACTGTCAGGTGT |
| | | GGGATAGTAGTAGTGATCATTATGTCTTC |
| | | GGAACTGGGACCAAGGTCACCGTCCTAG |
| SARS-CoV-2 | 91 | QVQLQESGPGLVKPSQTLSLTCTVSGGSISSG |
| S2A10-v1 | | DYYWSWIRQPPGKGLEWIGYIYYSGSTYYNP |
| mAb VH (aa) | | SLKSRVTISVDSSKNQFSLRLSSVTAADTAVY |
| | | YCARGIKDAYCGGDCYHAFDIWGQGTMVT |
| | | VSS |
| SARS-CoV-2 | 92 | GGSISSGDYY |
| S2A10-v1 | | |
| mAb CDRH1 | | |
| (aa) | | |
| SARS-CoV-2 | 93 | IYYSGST |
| S2A10-v1 | | |
| mAb CDRH2 | | |
| (aa) | | |
| SARS-CoV-2 | 94 | ARGIKDAYCGGDCYHAFDI |
| S2A10-v1 | | |
| mAb CDRH3 | | |
| (aa) | | |
| SARS-CoV-2 | 95 | SYVLTQPPSVSVAPGQTARITCGGNNIGSKSV |
| S2A10-v1 | | HWYQQKPGQAPVLVVYDDSDRPSGIPERFSG |
| mAb VL(VK) | | SNSGNTATLTISRVEAGDEADYYCQVWDSTS |
| (aa) | | DHPNVFGTGTKVTVL |
| SARS-CoV-2 | 96 | NIGSKS |
| S2A10-v1 | | |
| mAb CDRL1 | | |
| (aa) | | |
| SARS-CoV-2 | 97 | DDS |
| S2A10-v1 | | |
| mAb CDRL2 | | |
| (aa) | | |
| SARS-CoV-2 | 98 | QVWDSTSDHPNV |
| S2A10-v1 | | |
| mAb CDRL3 | | |
| (aa) | | |
| SARS-CoV-2 | 99 | CAGGTGCAGCTGCAGGAGTCGGGCCCAGG |
| S2A10-v1 | | ACTGGTGAAGCCTTCACAGACCCTGTCCCT |
| mAb VH (nt) | | CACCTGCACTGTCTCTGGTGGCTCCATCAG |
| | | CAGTGGTGATTACTACTGGAGTTGGATCC |
| | | GCCAGCCCCCAGGGAAGGGCCTGGAGTGG |
| | | ATTGGGTACATCTATTACAGTGGGAGCAC |
| | | CTACTACAACCCGTCCCTCAAGAGTCGAGT |
| | | TACCATATCAGTAGACTCGTCCAAGAACCA |
| | | GTTCTCCCTGAGGCTGAGCTCTGTGACTGCC |
| | | GCAGACACGGCCGTGTATTACTGTGCCAGA |
| | | GGGATTAAGGACGCATATTGTGGTGGTG |
| | | ATTGCTACCATGCTTTTGATATCTGGGGC |
| | | CAAGGGACAATGGTCACCGTCTCTTCAG |
| SARS-CoV-2 | 100 | TCCTATGTGCTGACTCAGCCACCCTCGGTGT |
| S2A10-v1 | | CAGTGGCCCCAGGACAGACGGCCAGGATTA |
| mAb VL(VK) | | CCTGTGGGGGAAACAACATTGGAAGTAAA |
| (nt) | | AGTGTGCACTGGTACCAGCAGAAGCCAGG |
| | | CCAGGCCCCTGTGCTGGTCGTCTATGATGA |
| | | TAGCGACCGGCCCTCAGGGATCCCTGAGCG |
| | | ATTCTCTGGCTCCAACTCTGGGAACACGGC |
| | | CACCCTGACCATCAGCAGGGTCGAAGCCGG |
| | | GGATGAGGCCGACTATTACTGTCAGGTGT |
| | | GGGATAGTACTAGTGATCATCCAAATGT |
| | | CTTCGGAACTGGGACCAAGGTCACCGTCCT |
| | | AG |
| SARS-CoV-2 | 101 | QVQLQESGPGLVKPSGTLSLTCAVSGGSISSS |
| S2A15-v1 | | NWWSWVRQPPGKGLEWIGEIYHSGNTNYNP |
| mAb VH (aa) | | SLKSRVTISVDKSKNQFSLKLTSVTAADTAVY |
| | | YCASRYCSSTSCPNWFDPWGQGTLVTVSS |
| SARS-CoV-2 | 102 | GGSISSSNW |
| S2A15-v1 | | |
| mAb CDRH1 | | |
| (aa) | | |
| SARS-CoV-2 | 103 | IYHSGNT |
| S2A15-v1 | | |
| mAb CDRH2 | | |
| (aa) | | |
| SARS-CoV-2 | 104 | ASRYCSSTSCPNWFDP |
| S2A15-v1 | | |
| mAb CDRH3 | | |
| (aa) | | |
| SARS-CoV-2 | 105 | QSALTQPASVSGSPGQSITISCTGTSSDVGSYN |
| S2A15-v1 | | LVSWYQQHPGKAPKFMIYEGSKRPSGVSNRF |
| mAb VL (aa) | | SGSKSGNTASLTISGLQAEDEADYYCCSYAG |
| | | SSTWVFGGGTKLTVL |
| SARS-CoV-2 | 106 | SSDVGSYNL |
| S2A15-v1 | | |
| mAb CDRL1 | | |
| (aa) | | |
| SARS-CoV-2 | 107 | EGS |
| S2A15-v1 | | |
| mAb CDRL2 | | |
| (aa) | | |
| SARS-CoV-2 | 108 | CSYAGSSTWV |
| S2A15-v1 | | |
| mAb CDRL3 | | |
| (aa) | | |
| SARS-CoV-2 | 109 | CAGGTGCAGCTGCAGGAGTCGGGCCCAGG |
| S2A15-v1 | | ACTGGTGAAGCCTTCGGGGACCCTGTCCCT |
| mAb VH (nt) | | CACCTGCGCTGTCTCTGGTGGCTCCATCAG |
| | | CAGTAGTAACTGGTGGAGTTGGGTCCGCC |
| | | AGCCCCCAGGGAAGGGGCTGGAGTGGATT |
| | | GGGGAAATCTATCATAGTGGGAACACCA |
| | | ACTACAACCCGTCCCTCAAGAGTCGAGTCA |
| | | CCATATCAGTAGACAAGTCCAAGAACCAGT |
| | | TCTCCCTGAAGCTGACCTCTGTGACCGCCG |
| | | CGGACACGGCCGTGTATTACTGTGCGAGCC |
| | | GATATTGTAGTAGTACCAGTTGCCCCAAC |
| | | TGGTTCGACCCCTGGGGCCAGGGAACCCT |
| | | GGTCACCGTCTCCTCAG |
| SARS-CoV-2 | 110 | CAGTCTGCCCTGACTCAGCCTGCCTCCGTGT |
| S2A15-v1 | | CTGGGTCTCCTGGACAGTCGATCACCATCT |
| mAb VL (nt) | | CCTGCACTGGAACCAGCAGTGATGTTGGG |
| | | AGTTATAACCTTGTCTCCTGGTACCAACAG |
| | | CACCCAGGCAAAGCCCCCAAATTCATGATT |
| | | TATGAGGGCAGTAAGCGGCCCTCAGGGGT |
| | | TTCTAATCGCTTCTCTGGCTCCAAGTCTGGC |
| | | AACACGGCCTCCCTGACAATCTCTGGGCTC |
| | | CAGGCTGAGGACGAGGCTGATTATTACTGC |
| | | TGCTCATATGCAGGTAGTAGCACTTGGG |
| | | TGTTCGGCGGAGGGACCAAGCTGACCGTCC |
| | | TAG |
| SARS-CoV-2 | 111 | QVQLVESGGGVVQPGRSLRLSCAASGFTFSN |
| S2B2-v1 mAb | | FGMHWVRQAPGKGLEWVAVISYDGNNKFY |
| VH (aa) | | ADSVKGRFTISRDNSKNTLYLQMNSLRAEDT |
| | | AVYYCAKPTVSFGVVIDAFDIWGQGTMVTV |
| | | SS |
| SARS-CoV-2 | 112 | GFTFSNFG |
| S2B2-v1 mAb | | |
| CDRH1 (aa) | | |
| SARS-CoV-2 | 113 | ISYDGNNK |
| S2B2-v1 mAb | | |
| CDRH2 (aa) | | |
| SARS-CoV-2 | 114 | AKPTVSFGVVIDAFDI |
| S2B2-v1 mAb | | |
| CDRH3 (aa) | | |
| SARS-CoV-2 | 115 | DIVMTQSPDSLAVSLGERASINCKSSQSVLYS |
| S2B2-v1 mAb | | SNNKNYLAWYQQKPGQPPKLLIYWASTRES |
| VL(VK) (aa) | | GVPDRFSGSGSGTDFTLTISSLQAEDVAVYYC |
| | | QQYYRIITFGQGTRLEIK |
| SARS-CoV-2 | 116 | QSVLYSSNNKNY |
| S2B2-v1 mAb | | |
| CDRL1 (aa) | | |
| SARS-CoV-2 | 117 | WAS |
| S2B2-v1 mAb | | |
| CDRL2 (aa) | | |
| SARS-CoV-2 | 118 | QQYYRIIT |
| S2B2-v1 mAb | | |
| CDRL3 (aa) | | |
| SARS-CoV-2 | 119 | CAGGTGCAGCTGGTGGAGTCTGGGGGAGGC |
| S2B2-v1 mAb | | GTGGTCCAGCCTGGGAGGTCCCTGAGACTC |
| VH (nt) | | TCCTGTGCAGCCTCTGGATTCACGTTCAGT |
| | | AACTTTGGCATGCACTGGGTCCGCCAGGCT |
| | | CCAGGCAAGGGGCTGGAGTGGGTGGCAGTT |
| | | ATATCATATGATGGAAATAATAAATTCTA |
| | | TGCAGACTCCGTGAAGGGCCGATTCACCAT |
| | | CTCCAGAGACAATTCCAAGAACACGCTGTA |
| | | TCTGCAAATGAACAGCCTGAGAGCTGAGGA |
| | | CACGGCTGTGTATTACTGTGCGAAACCCAC |
| | | CGTATCTTTTGGAGTGGTTATTGATGCTT |
| | | TTGATATCTGGGGCCAAGGGACAATGGTC |
| | | ACCGTCTCTTCAG |
| SARS-CoV-2 | 120 | GACATCGTGATGACCCAGTCTCCAGACTCC |
| S2B2-v1 mAb | | CTGGCTGTGTCTCTGGGCGAGAGGGCCAGC |
| VL(VK) (nt) | | ATCAACTGCAAGTCCAGCCAGAGTGTTTT |
| | | ATACAGCTCCAACAATAAGAACTACTTAG |
| | | CTTGGTACCAGCAGAAACCAGGACAGCCTC |
| | | CTAAGCTGCTCATTTACTGGGCATCTACCC |
| | | GGGAATCCGGGGTCCCTGACCGATTCAGTG |
| | | GCAGCGGGTCTGGGACAGATTTCACTCTCA |
| | | CCATCAGCAGCCTGCAGGCTGAAGATGTGG |
| | | CAGTTTATTACTGTCAGCAATATTATAGAA |
| | | TTATCACCTTCGGCCAAGGGACACGACTGG |
| | | AGATTAAAC |
| SARS-CoV-2 | 121 | EVQLLESGGGLVQPGGSLRLSCAGSGFSVSN |
| S2F1-v1 mAb | | HAMSWVRQAPGKGLEWVSAIGGSDSTTYY |
| VH (aa) | | ADSVKGRFAISRDNSKNTLYLQMNSLRADDT |
| | | AIYYCAKDNYTSTWFPFDYWGQGTQVIVSS |
| SARS-CoV-2 | 122 | GFSVSNHA |
| S2F1-v1 mAb | | |
| CDRH1 (aa) | | |
| SARS-CoV-2 | 123 | IGGSDSTT |
| S2F1-v1 mAb | | |
| CDRH2 (aa) | | |
| SARS-CoV-2 | 124 | AKDNYTSTWFPFDY |
| S2F1-v1 mAb | | |
| CDRH3 (aa) | | |
| SARS-CoV-2 | 125 | DVVMTQSPLSLPVTLGQPASISCRSSHSLVHG |
| S2F1-v1 mAb | | DGNTYLNWFQQRPGQSPRRLIHKVSNRDSGV |
| VL (aa) | | PDRFSGSGSGTDFTLKISRVEAEDVGVYYCM |
| | | QGSYWPPWTFGQGTKVEIN |
| SARS-CoV-2 | 126 | HSLVHGDGNTY |
| S2F1-v1 mAb | | |
| CDRL1 (aa) | | |
| SARS-CoV-2 | 127 | KVS |
| S2F1-v1 mAb | | |
| CDRL2 (aa) | | |
| SARS-CoV-2 | 128 | MQGSYWPPWT |
| S2F1-v1 mAb | | |
| CDRL3 (aa) | | |
| SARS-CoV-2 | 129 | GAGGTGCAACTGTTGGAGTCTGGGGGAGGC |
| S2F1-v1 mAb | | TTGGTACAGCCTGGGGGGTCCCTGAGACTC |
| VH (nt) | | TCCTGCGCAGGCTCTGGATTCAGTGTTAG |
| | | CAACCATGCCATGAGCTGGGTCCGCCAGG |
| | | CTCCAGGGAAGGGGCTGGAGTGGGTCTCAG |
| | | CAATTGGTGGAAGTGACAGTACTACATAC |
| | | TACGCAGACTCTGTGAAGGGCCGGTTCGCC |
| | | ATCTCCAGAGACAATTCCAAGAACACGCTG |
| | | TATCTGCAAATGAACAGCCTGAGAGCCGAC |
| | | GACACGGCCATATATTACTGTGCCAAAGAC |
| | | AACTATACCAGTACCTGGTTCCCCTTTGA |
| | | CTACTGGGGCCAGGGAACCCAGGTCATCGT |
| | | CTCCTCAG |
| SARS-CoV-2 | 130 | GATGTTGTGATGACTCAGTCTCCACTCTCTC |
| S2F1-v1 mAb | | TGCCCGTCACCCTTGGGCAGCCGGCCTCCA |
| VL(VK) (nt) | | TCTCCTGCAGGTCTAGTCACAGTCTCGTAC |
| | | ACGGTGATGGAAACACCTACTTGAATTGG |
| | | TTTCAGCAGAGGCCTGGCCAATCTCCAAGG |
| | | CGCCTGATTCATAAGGTTTCTAACCGGGAC |
| | | TCTGGGGTCCCTGACAGATTCAGCGGCAGT |
| | | GGGTCAGGCACTGATTTCACACTGAAGATC |
| | | AGCAGGGTGGAGGCTGAGGATGTTGGGGTT |
| | | TATTACTGCATGCAAGGTTCATATTGGCC |
| | | CCCGTGGACGTTCGGCCAAGGGACCAAGG |
| | | TGGAAATCAATC |
| SARS-CoV-2 | 131 | GAGGTGCAGCTGGTGGAGTCTGGGGGAGG |
| S2H7-v1 | | CTTGGTCCAGCCTGGGGGGTCCCTGAGACT |
| mAb VH (nt) | | CTCCTGTGCAGCCTCTGGATTCATCGTCAG |
| | | TAGTAATTACATGAGCTGGGTCCGCCAGGC |
| | | TCCAGGGAAGGGGCTGGAGTGGGTCTCAGT |
| | | TATTTATAGTGGTGGTAGCACATACTACG |
| | | CAGACTCCGTGAAGGGCAGATTCACCATCT |
| | | CCAGAGACAATTCCAAGAACACGCTGTATC |
| | | TTCAAATGAACAGCCTGAGAGCCGAGGACA |
| | | CGTCTGTGTATTACTGTGCGAGAGATCGG |
| | | CAGTATAGTGGGAGCCCCAGCTTTGACT |
| | | ACTGGGGCCAGGGAACCCTGGTCACCGTCT |
| | | CCTCAG |
| SARS-CoV-2 | 132 | GACATCCAGATGACCCAGTCTCCATCCTCC |
| S2H7-v1 | | CTGTCTGCATCTGTAGGAGACAGAGTCACC |
| mAb VL(VK) | | ATCACTTGCCGGGCAAGTCAGAGCATTAG |
| (nt) | | CAGCTATTTAAATTGGTATCAGCAGAAACC |
| | | AGGGAAAGCCCCTAAGCTCCTGATCTATAC |
| | | TGCATCCAGTTTGCAAAGTGGGGTCCCATC |
| | | AAGGTTCAGTGGCAGTGGATCTGGGACAGA |
| | | TTTCACTCTCACCATCAGCAGTCTGCAACCT |
| | | GAAGATTTTGCAACTTACTACTGTCAACAG |
| | | AGTTACAGTACCCCTGGGCTCACTTTCGG |
| | | CGGAGGGACCAAGGTGGAGATCAAAC |
| SARS-CoV-2 | 133 | GAGGTGCAGCTGGTGGAGTCTGGGGGAGA |
| S2H13-v1 | | CTCGGTCCAGCCTGGGGGGTCCCTGAGACT |
| mAb VH (nt) | | CTCCTGTGCAGCCGCTGGATTCACCTTTAG |
| | | TAGCTATTGGATGAACTGGGTCCGCCAGG |
| | | CACCAGGGAAGGGGCTGGAGTGGGTGGCC |
| | | AACATAAAGCAAGATGGAAGTGAGAAAT |
| | | ACTATGTGGACTCTGTGAAGGGCCGATTCA |
| | | CCATCTCCAGAGACAACGCCAAGAACTCAC |
| | | TGTATCTACAAATGAACAGCCTGAGAGCCG |
| | | AGGACACGGCCGTGTATTACTGTGCGCTAT |
| | | CATCAGGATATAGTGGCTACGCAGGTAA |
| | | CTACTGGGGCCAGGGAACCCTGGTCACCGT |
| | | CTCCTCAG |
| SARS-CoV-2 | 134 | CAGGCTGTGGTGACTCAGGAGCCCTCACTG |
| S2H13-v1 | | ACTGTGTCCCCAGGAGGGACAGTCACTCTC |
| mAb VL (nt) | | ACCTGTGGCTCCAGCACTGGAGCTGTCAC |
| | | CAGTGGTCATTATCCCTACTGGTTCCAGCA |
| | | GAAGCCTGGCCAAGCCCCCAGGACACTGAT |
| | | TTATGATACAAGCAACAAACACTCCTGGAC |
| | | CCCTGCCCGATTCTCAGGCTCCCTCCTTGGG |
| | | GGCAAAGCTGCCCTGACCCTTTCGGGTGCG |
| | | CGGCCTGAGGATGAGGCTGAGTATTACTGC |
| | | TTGCTCTCCTATAGTGGTGCTCGGGGGG |
| | | TGTTCGGCGGAGGGACCAAGCTGACCGTCC |
| | | TAG |
| SARS-CoV-2 | 135 | QVQLVESGGGVVQPGGSLRLSCVASGFTFSD |
| S2R5-v1 mAb | | YGVNWVRQAPGKGLDWVAYIQYDGSNKYY |
| VH (aa) | | ADSVKGRFTISRDNFKNTLHLQMNSLRAEDT |
| | | AVYFCAKLSVAGSFGPFDIWGQGTLVTVSS |
| SARS-CoV-2 | 136 | GFTFSDYG |
| S2R5-v1 mAb | | |
| CDRH1 (aa) | | |
| SARS-CoV-2 | 137 | IQYDGSNK |
| S2R5-v1 mAb | | |
| CDRH2 (aa) | | |
| SARS-CoV-2 | 138 | AKLSVAGSFGPFDI |
| S2R5-v1 mAb | | |
| CDRH3 (aa) | | |
| SARS-CoV-2 | 139 | QSALTQPASVSGSPGQSITISCTGTSNDVGGF |
| S2R5-v1 mAb | | NYVSWYQQHPGKAPKLLIYEVSSRPSGVSTR |
| VL (aa) | | FSGSKSANTASLTVSGLQAEDEADYYCGSYS |
| | | STNTLVVFGSGTKVTVL |
| SARS-CoV-2 | 140 | SNDVGGFNY |
| S2R5-v1 mAb | | |
| CDRL1 (aa) | | |
| SARS-CoV-2 | 141 | EVS |
| S2R5-v1 mAb | | |
| CDRL2 (aa) | | |
| SARS-CoV-2 | 142 | GSYSSTNTLVV |
| S2R5-v1 mAb | | |
| CDRL3 (aa) | | |
| SARS-CoV-2 | 143 | CAGGTGCAGTTGGTGGAGTCTGGGGGAGGC |
| S2R5-v1 mAb | | GTGGTCCAGCCTGGGGGGTCCCTGAGACTC |
| VH (nt) | | TCCTGTGTAGCGTCCGGGTTCACCTTCAGT |
| | | GACTATGGAGTTAACTGGGTCCGCCAGGCT |
| | | CCAGGCAAGGGGCTGGACTGGGTGGCATAT |
| | | ATACAATATGATGGAAGTAATAAATACTA |
| | | TGCGGACTCCGTGAAGGGCCGATTCACCAT |
| | | CTCCCGAGACAATTTCAAGAACACGCTGCA |
| | | TCTTCAAATGAACAGCCTGAGAGCCGAGGA |
| | | CACGGCTGTGTATTTCTGTGCGAAGCTTTC |
| | | AGTGGCTGGTTCTTTCGGTCCTTTTGATA |
| | | TCTGGGGCCAAGGGACATTGGTCACCGTCT |
| | | CTTCAG |
| SARS-CoV-2 | 144 | CAGTCTGCCCTGACTCAGCCTGCCTCCGTGT |
| S2R5-v1 mAb | | CTGGGTCTCCTGGACAGTCGATCACCATCT |
| VL (nt) | | CCTGCACTGGAACCAGCAATGACGTTGGT |
| | | GGTTTTAACTATGTCTCCTGGTACCAACAA |
| | | CACCCAGGCAAAGCCCCCAAACTCCTGATT |
| | | TATGAGGTCAGTAGTCGGCCCTCAGGGGT |
| | | CTCTACTCGCTTCTCTGGCTCCAAGTCTGCC |
| | | AACACGGCCTCCCTGACCGTCTCTGGGCTC |
| | | CAGGCTGAGGACGAGGCTGATTATTACTGC |
| | | GGCTCATATTCAAGCACCAACACTCTCGT |
| | | TGTCTTCGGAAGTGGGACCAAGGTCACCGT |
| | | CCTAG |
| SARS-CoV-2 | 145 | QVQLQESGPGLVRPSETLSLTCTVSSASIPTG |
| S2R7-v1 mAb | | THYWGWIRQPPGQGLEWIGSISNIGYSFYNPS |
| VH (aa) | | LKSRVSISIDTSKKQFSLKVRSVTAADTAVYY |
| | | CVRPTNEYGGFWFDRWGQGTLVTVSS |
| SARS-CoV-2 | 146 | SASIPTGTHY |
| S2R7-v1 mAb | | |
| CDRH1 (aa) | | |
| SARS-CoV-2 | 147 | ISNIGYS |
| S2R7-v1 mAb | | |
| CDRH2 (aa) | | |
| SARS-CoV-2 | 148 | VRPTNEYGGFWFDR |
| S2R7-v1 mAb | | |
| CDRH3 (aa) | | |
| SARS-CoV-2 | 149 | SYVLTQPPSVSVAPGKTARFTCGGDNIGSKR |
| S2R7-v1 mAb | | VHWYQQKPGQAPVLVIYYDADRPSGIPERFS |
| VL (aa) | | GSKSGSTATLTISRVEAGDEADYYCQVWEST |
| | | RDHVIFGGGTKLTVL |
| SARS-CoV-2 | 150 | NIGSKR |
| S2R7-v1 mAb | | |
| CDRL1 (aa) | | |
| SARS-CoV-2 | 151 | YDA |
| S2R7-v1 mAb | | |
| CDRL2 (aa) | | |
| SARS-CoV-2 | 152 | QVWESTRDHVI |
| S2R7-v1 mAb | | |
| CDRL3 (aa) | | |
| SARS-CoV-2 | 153 | CAGGTGCAACTGCAGGAGTCGGGCCCAGG |
| S2R7-v1 mAb | | ACTGGTGAGGCCTTCGGAGACCCTGTCGCT |
| VH (nt) | | CACCTGCACTGTCTCTAGTGCCTCCATCCC |
| | | CACTGGTACTCACTATTGGGGCTGGATCC |
| | | GCCAGCCCCCAGGGCAGGGACTGGAGTGG |
| | | ATTGGGAGTATCTCTAATATTGGGTACAG |
| | | CTTCTACAACCCGTCCCTCAAGAGTCGAGT |
| | | CAGCATATCCATTGACACGTCCAAGAAGCA |
| | | GTTCTCCCTGAAAGTGAGGTCTGTGACCGC |
| | | CGCGGACACGGCTGTCTACTACTGTGTGAG |
| | | ACCTACGAACGAATACGGTGGTTTCTGG |
| | | TTCGACCGCTGGGGCCAGGGAACCCTGGT |
| | | CACGGTCTCCTCAG |
| SARS-CoV-2 | 154 | TCCTATGTGCTGACTCAGCCACCCTCAGTGT |
| S2R7-v1 mAb | | CAGTGGCCCCTGGAAAGACGGCCAGATTTA |
| VL (nt) | | CCTGTGGGGGAGACAACATTGGAAGTAAA |
| | | AGAGTGCACTGGTACCAGCAGAAGCCAGG |
| | | CCAGGCCCCTGTTCTGGTCATCTATTATGA |
| | | TGCCGACCGGCCCTCAGGGATCCCTGAGCG |
| | | ATTCTCTGGCTCCAAGTCTGGGAGCACGGC |
| | | CACCCTGACCATCAGCAGGGTCGAAGCCGG |
| | | GGATGAGGCCGACTATTACTGTCAGGTGT |
| | | GGGAAAGTACTCGTGATCATGTGATTTTC |
| | | GGCGGAGGGACCAAGCTGACCGTCCTAG |
| SARS-CoV-2 | 155 | EVQLVESGGGVVQPGRSLRLSCAASGFTFSN |
| S2B2-v2 mAb | | FGMHWVRQAPGKGLEWVAVISYDGNNKFY |
| VH (aa) | | ADSVKGRFTISRDNSKNTLYLQMNSLRAEDT |
| | | AVYYCSKPTVSFGVVIDAFDIWGQGTMVTV |
| | | SS |
| SARS-CoV-2 | 156 | SKPTVSFGVVIDAFDI |
| S2B2-v2 mAb | | |
| CDRH3 (aa) | | |
| SARS-CoV-2 | 157 | GAGGTGCAGCTGGTGGAGTCTGGGGGAGG |
| S2B2-v2 mAb | | CGTGGTCCAGCCTGGGAGGTCCCTGAGACT |
| VH (nt) | | CTCCTGTGCAGCCTCTGGATTCACGTTCAG |
| | | TAACTTTGGCATGCACTGGGTCCGCCAGGC |
| | | TCCAGGCAAGGGGCTGGAGTGGGTGGCAGT |
| | | TATATCATATGATGGAAATAATAAATTCT |
| | | ATGCAGACTCCGTGAAGGGCCGATTCACCA |
| | | TCTCCAGAGACAATTCCAAGAACACGCTGT |
| | | ATCTGCAAATGAACAGCCTGAGAGCTGAGG |
| | | ACACGGCTGTGTATTACTGTTCGAAACCCA |
| | | CCGTATCTTTTGGAGTGGTTATTGATGCT |
| | | TTTGATATCTGGGGCCAAGGGACAATGGT |
| | | CACCGTCTCTTCAG |
| SARS-CoV-2 | 158 | QITLKESGPGLVKPSGTLSLTCAVSGGSISSSN |
| S2A15-v2 | | WWSWVRQPPGKGLEWIGEIYHSGNTNYNPS |
| mAb VH (aa) | | LKSRVTISVDKSKNQFSLKLTSVTAADTAVY |
| | | YCASRYCSSTSCPNWFDPWGQGTLVTVSS |
| SARS-CoV-2 | 159 | CAGATCACCTTGAAGGAGTCGGGCCCAGGA |
| S2A15-v2 | | CTGGTGAAGCCTTCGGGGACCCTGTCCCTC |
| mAb VH (nt) | | ACCTGCGCTGTCTCTGGTGGCTCCATCAG |
| | | CAGTAGTAACTGGTGGAGTTGGGTCCGCC |
| | | AGCCCCCAGGGAAGGGGCTGGAGTGGATT |
| | | GGGGAAATCTATCATAGTGGGAACACCA |
| | | ACTACAACCCGTCCCTCAAGAGTCGAGTCA |
| | | CCATATCAGTAGACAAGTCCAAGAACCAGT |
| | | TCTCCCTGAAGCTGACCTCTGTGACCGCCG |
| | | CGGACACGGCCGTGTATTACTGTGCGAGCC |
| | | GATATTGTAGTAGTACCAGTTGCCCCAAC |
| | | TGGTTCGACCCCTGGGGCCAGGGAACCCT |
| | | GGTCACCGTCTCCTCAG |
| SARS-CoV-2 | 160 | GFIVSSNYMS |
| S2H7-v1.1 | | |
| mAb CDRH1 | | |
| (aa) | | |
| SARS-CoV-2 | 161 | VIYSGGSTYYADSVKG |
| S2H7-v1.1 | | |
| mAb CDRH2 | | |
| (aa) | | |
| SARS-CoV-2 | 162 | DRQYSGSPSFDY |
| S2H7-v1.1 | | |
| mAb CDRH3 | | |
| (aa) | | |
| SARS-CoV-2 | 163 | RASQSISSYLN |
| S2H7-v1.1 | | |
| mAb CDRL1 | | |
| (aa) | | |
| SARS-CoV-2 | 164 | TASSLQS |
| S2H7-v1.1 | | |
| mAb CDRL2 | | |
| (aa) | | |
| SARS-CoV-2 | 165 | QQSYSTPGLT |
| S2H7-v1.1 | | |
| mAb CDRL3 | | |
| (aa) | | |
| SARS-CoV-2 | 166 | GFTFSSYWMN |
| S2H13-v1.1 | | |
| mAb CDRH1 | | |
| (aa) | | |
| SARS-CoV-2 | 167 | NIKQDGSEKYYVDSVKG |
| S2H13-v1.1 | | |
| mAb CDRH2 | | |
| (aa) | | |
| SARS-CoV-2 | 168 | SSGYSGYAGNY |
| S2H13-v1.1 | | |
| mAb CDRH3 | | |
| (aa) | | |
| SARS-CoV-2 | 169 | GSSTGAVTSGHYPY |
| S2H13-v1.1 | | |
| mAb CDRL1 | | |
| (aa) | | |
| SARS-CoV-2 | 170 | DTSNKHS |
| S2H13-v1.1 | | |
| mAb CDRL2 | | |
| (aa) | | |
| SARS-CoV-2 | 171 | DTSNKHS |
| S2H13-v1.1 | | |
| mAb CDRL3 | | |
| (aa) | | |
| SARS-CoV-2 | 172 | QVQLVQSGAEVKKPGASVKVSCKASGYPFT |
| S309 mAb | | SYGISWVRQAPGQGLEWMGWISTYNGNTNY |
| VH (aa) | | AQKFQGRVTMTTDTSTTTGYMELRRLRSDDT |
| | | AVYYCARDYTRGAWFGESLIGGFDNWGQG |
| | | TLVTVSS |
| SARS-CoV-2 | 173 | GYPFTSYG |
| S309 mAb | | |
| CDRH1 (aa) | | |
| SARS-CoV-2 | 174 | ISTYNGNT |
| S309 mAb | | |
| CDRH2 (aa) | | |
| SARS-CoV-2 | 175 | ARDYTRGAWFGESLIGGFDN |
| S309 mAb | | |
| CDRH3 (aa) | | |
| SARS-CoV-2 | 176 | EIVLTQSPGTLSLSPGERATLSCRASQTVSSTS |
| S309 mAb | | LAWYQQKPGQAPRLLIYGASSRATGIPDRFSG |
| VL (aa) | | SGSGTDFTLTISRLEPEDFAVYYCQQHDTSLT |
| | | FGGGTKVEIK |
| SARS-CoV-2 | 177 | QTVSSTS |
| S309 mAb | | |
| CDRL1 (aa) | | |
| SARS-CoV-2 | 178 | GAS |
| S309 mAb | | |
| CDRL2 (aa) | | |
| SARS-CoV-2 | 179 | QQHDTSLT |
| S309 mAb | | |
| CDRL3 (aa) | | |
| SARS-CoV-2 | 180 | EVQLVQSGAEVKKPGSSVKVSCKASGGVFSS |
| S2N3-v1 | | YAISWVRQAPGQGLEWMGGIIPLFVTPTYAQ |
| mAb VH (aa) | | KFQGRVTITADESTTTASMELSSLTSDDTAVY |
| | | YCARDRSGYSGSWPVPNFAFHIWGQGTLVT |
| | | VSS |
| SARS-CoV-2 | 181 | GGVFSSYA |
| S2N3-v1 | | |
| mAb CDRH1 | | |
| (aa) | | |
| SARS-CoV-2 | 182 | IIPLFVTP |
| S2N3-v1 | | |
| mAb CDRH2 | | |
| (aa) | | |
| SARS-CoV-2 | 183 | ARDRSGYSGSWPVPNFAFHI |
| S2N3-v1 | | |
| mAb CDRH3 | | |
| (aa) | | |
| SARS-CoV-2 | 184 | EIVLTQSPGTLSLSPGERATLSCRASQSVSSRSLA |
| S2N3-v1 | | WYQQKPGQAPRLLIYGASSRATGIPDRFSGSGSGT |
| mAb VL(VK) | | DFTLTISRLEPEDFAVYYCQQYGTSPRSFGPGTK |
| (aa) | | VDIK |
| SARS-CoV-2 | 185 | QSVSSRS |
| S2N3-v1 | | |
| mAb CDRL1 | | |
| (aa) | | |
| SARS-CoV-2 | 186 | GAS |
| S2N3-v1 | | |
| mAb CDRL2 | | |
| (aa) | | |
| SARS-CoV-2 | 187 | QQYGTSPRS |
| S2N3-v1 | | |
| mAb CDRL3 | | |
| (aa) | | |
| SARS-CoV-2 | 188 | GAGGTGCAGCTGGTGCAGTCTGGGGCTGAG |
| S2N3-v1 | | GTGAAGAAGCCTGGGTCCTCGGTGAAGGTC |
| mAb VH (nt) | | TCCTGCAAGGCTTCTGGAGGCGTCTTCAG |
| | | CAGCTATGCTATCAGCTGGGTGCGACAGG |
| | | CCCCTGGACAAGGGCTTGAGTGGATGGGGG |
| | | GGATCATCCCTCTCTTTGTTACACCAACT |
| | | TATGCACAGAAGTTCCAGGGCAGAGTCACG |
| | | ATTACCGCGGACGAATCCACGACCACAGCC |
| | | TCCATGGAGCTGAGCAGCCTGACATCTGAC |
| | | GACACGGCCGTCTATTACTGTGCGCGAGAT |
| | | CGTTCCGGATATAGCGGCAGCTGGCCAG |
| | | TCCCGAACTTTGCTTTTCATATCTGGGGC |
| | | CAAGGGACACTGGTCACCGTCTCTTCAG |
| SARS-CoV-2 | 189 | GAAATTGTGTTGACGCAGTCTCCAGGCACC |
| S2N3-v1 | | CTGTCTTTGTCTCCAGGGGAAAGAGCCACC |
| mAb VL(VK) | | CTCTCCTGCAGGGCCAGTCAGAGTGTTAG |
| (nt) | | CAGCAGGTCCTTAGCCTGGTACCAGCAGA |
| | | AACCTGGCCAGGCTCCCAGGCTCCTCATCT |
| | | ATGGTGCATCCAGCAGGGCCACTGGCATC |
| | | CCGGACAGGTTCAGTGGCAGTGGGTCTGGG |
| | | ACAGACTTCACTCTCACCATCAGCAGACTG |
| | | GAGCCTGAAGATTTTGCAGTGTATTACTGT |
| | | CAGCAGTATGGTACCTCACCTCGGTCTTT |
| | | CGGCCCTGGGACCAAAGTGGATATCAAAC |
| SARS-CoV-2 | 190 | QVQLVQSGAEVKRPGSSVKVSCKASGGVFSS |
| S2N6-v1 | | FAISWVRQAPGQGLEWMGGIIPLFVKPDYA |
| mAb VH (aa) | | QKFQDRVTITADESTTTAYMELRSLKSDDTA |
| | | VYYCARDHSGYSGSWPVPNYPFDIWGQGT |
| | | MVTVSS |
| SARS-CoV-2 | 191 | GGVFSSFA |
| S2N6-v1 | | |
| mAb CDRH1 | | |
| (aa) | | |
| SARS-CoV-2 | 192 | IIPLFVKP |
| S2N6-v1 | | |
| mAb CDRH2 | | |
| (aa) | | |
| SARS-CoV-2 | 193 | ARDHSGYSGSWPVPNYPFDI |
| S2N6-v1 | | |
| mAb CDRH3 | | |
| (aa) | | |
| SARS-CoV-2 | 194 | EIVLTQSPGTLSLSPGERAILSCRASQSVSSNSLA |
| | | WYQQIPGQPPRLLIYGASSRATGIPDRFSGSGSGT |
| S2N6-v1 | | DFTLSISRLEPEDFAVYYCQQYGGSTRSFGPGTK |
| mAb VL(VK) | | VDIK |
| (aa) | | |
| SARS-CoV-2 | 195 | QSVSSNS |
| S2N6-v1 | | |
| mAb CDRL1 | | |
| (aa) | | |
| SARS-CoV-2 | 196 | GAS |
| S2N6-v1 | | |
| mAb CDRL2 | | |
| (aa) | | |
| SARS-CoV-2 | 197 | QQYGGSTRS |
| S2N6-v1 | | |
| mAb CDRL3 | | |
| (aa) | | |
| SARS-CoV-2 | 198 | CAGGTGCAGCTGGTGCAGTCTGGGGCTGAG |
| S2N6-v1 | | GTGAAGAGGCCTGGGTCCTCCGTGAAGGTC |
| mAb VH (nt) | | TCCTGCAAGGCCTCTGGAGGCGTCTTCAG |
| | | CAGCTTTGCTATCAGCTGGGTGCGACAGG |
| | | CCCCTGGACAAGGGCTTGAGTGGATGGGAG |
| | | GGATCATCCCTTTGTTTGTTAAACCAGAC |
| | | TACGCACAGAAATTCCAGGACAGAGTCACG |
| | | ATTACCGCGGACGAATCAACGACCACAGCC |
| | | TACATGGAGCTGCGCAGCCTTAAATCTGAC |
| | | GACACGGCCGTTTATTACTGTGCGAGAGAT |
| | | CATTCCGGCTATAGTGGCAGCTGGCCGG |
| | | TGCCGAACTATCCTTTTGATATCTGGGGC |
| | | CAAGGGACAATGGTCACCGTCTCTTCAG |
| SARS-CoV-2 | 199 | GAAATTGTGTTGACGCAGTCTCCAGGCACC |
| S2N6-v1 | | CTGTCTTTGTCTCCTGGGGAAAGAGCCATC |
| mAb VL(VK) | | CTCTCCTGCAGGGCCAGTCAGAGTGTTAG |
| (nt) | | CAGCAACTCCTTAGCCTGGTACCAGCAGAT |
| | | TCCTGGCCAGCCTCCCAGGCTCCTCATCTAC |
| | | GGTGCATCCAGCAGGGCCACTGGCATCCC |
| | | AGACAGGTTCAGTGGCAGTGGGTCTGGGAC |
| | | AGACTTCACTCTCAGTATCAGCAGACTGGA |
| | | GCCTGAAGATTTTGCAGTATATTACTGTCA |
| | | GCAGTATGGTGGCTCAACTCGGTCTTTCG |
| | | GCCCTGGGACCAAAGTGGATATCAAAC |
| SARS-CoV-2 | 200 | QVQLVQSGAEVKKPGASVKVSCKASGYTFT |
| S2X2-v1 | | SYGVSWVRQAPGQGLEWMGWISAYNGNTN |
| mAb VH (aa) | | YAQNLQGRVTMTTDTSTSTAYMELRSLRSDD |
| | | TAVYYCARDAVITIFGVVIGKSGYYGMDV |
| | | WGQGTTVTVSS |
| SARS-CoV-2 | 201 | GYTFTSYG |
| S2X2-v1 | | |
| mAb CDRH1 | | |
| (aa) | | |
| SARS-CoV-2 | 202 | ISAYNGNT |
| S2X2-v1 | | |
| mAb CDRH2 | | |
| (aa) | | |
| SARS-CoV-2 | 203 | ARDAVITIFGVVIGKSGYYGMDV |
| S2X2-v1 | | |
| mAb CDRH3 | | |
| (aa) | | |
| SARS-CoV-2 | 204 | DVVMTQSPLSLPVTLGQPASISCRSSQSLVYS |
| S2X2-v1 | | DGNTYLNWFQQRPGQSPRRLIYKVSNRDSGV |
| mAb VL(VK) | | PDRFSGSGSGTDFTLEISRVEAEDVGVYYCM |
| (aa) | | QGTHWPAWTFGQGTKVEIK |
| SARS-CoV-2 | 205 | QSLVYSDGNTY |
| S2X2-v1 | | |
| mAb CDRL1 | | |
| (aa) | | |
| SARS-CoV-2 | 206 | KVS |
| S2X2-v1 | | |
| mAb CDRL2 | | |
| (aa) | | |
| SARS-CoV-2 | 207 | MQGTHWPAWT |
| S2X2-v1 | | |
| mAb CDRL3 | | |
| (aa) | | |
| SARS-CoV-2 | 208 | CAGGTGCAGCTGGTGCAGTCTGGAGCTGAG |
| S2X2-v1 | | GTGAAGAAGCCTGGGGCCTCAGTGAAGGTC |
| mAb VH (nt) | | TCCTGCAAGGCTTCTGGTTACACCTTTACC |
| | | AGCTATGGTGTCAGCTGGGTGCGACAGGC |
| | | CCCTGGACAAGGGCTTGAGTGGATGGGATG |
| | | GATCAGCGCTTACAATGGTAACACAAACT |
| | | ATGCACAGAACCTCCAGGGCAGAGTCACCA |
| | | TGACCACAGACACATCCACGAGCACAGCCT |
| | | ACATGGAGCTGAGGAGCCTGAGATCTGACG |
| | | ACACGGCCGTGTATTACTGTGCGAGAGAT |
| | | GCCGTGATTACGATTTTTGGAGTGGTTAT |
| | | TGGGAAATCGGGCTATTACGGTATGGAC |
| | | GTCTGGGGCCAAGGGACCACGGTCACCGTC |
| | | TCCTCA |
| SARS-CoV-2 | 209 | GATGTTGTGATGACTCAGTCTCCACTCTCCC |
| S2X2-v1 | | TGCCCGTCACCCTTGGACAGCCGGCCTCCA |
| mAb VL(VK) | | TCTCCTGCAGGTCTAGTCAAAGCCTCGTAT |
| (nt) | | ACAGTGATGGAAACACCTACTTGAATTGG |
| | | TTTCAGCAGAGGCCAGGCCAATCTCCAAGG |
| | | CGCCTAATTTATAAGGTTTCTAACCGGGAC |
| | | TCTGGGGTCCCAGACAGATTCAGCGGCAGT |
| | | GGGTCAGGCACTGATTTCACACTGGAAATC |
| | | AGCAGGGTGGAGGCTGAGGATGTTGGGGTT |
| | | TATTACTGCATGCAAGGTACACACTGGCC |
| | | TGCGTGGACGTTCGGCCAAGGGACCAAGG |
| | | TGGAGATCAAAC |
| SARS-CoV-2 | 210 | EVQLLESGGGLVQPGGSLRLSCAASGFTFTT |
| S2D8-v1 | | YAMSWVRQAPGKGLEWVSGISGSGGNTYH |
| mAb VH (aa) | | ADSVKGRFTISRDNSKSTLYLQMNSLRAEDT |
| | | AVYYCAKDLWFREILHGMDVWGEGTTVTV |
| | | SS |
| SARS-CoV-2 | 211 | GFTFTTYA |
| S2D8-v1 | | |
| mAb CDRH1 | | |
| (aa) | | |
| SARS-CoV-2 | 212 | ISGSGGNT |
| S2D8-v1 | | |
| mAb CDRH2 | | |
| (aa) | | |
| SARS-CoV-2 | 213 | AKDLWFREILHGMDV |
| S2D8-v1 | | |
| mAb CDRH3 | | |
| (aa) | | |
| SARS-CoV-2 | 214 | QSALTQPASVSGSPGQSITISCTGTSSDIGGYN |
| S2D8-v1 | | YVSWYQHHPGKAPKIMIYEVTNRPSGVSNRF |
| mAb VL (aa) | | SGSKSGNTASLTISGLQAEDEADYYCSSYTSS |
| | | NTYVFGTGTKVTVL |
| SARS-CoV-2 | 215 | SSDIGGYNY |
| S2D8-v1 | | |
| mAb CDRL1 | | |
| (aa) | | |
| SARS-CoV-2 | 216 | EVT |
| S2D8-v1 | | |
| mAb CDRL2 | | |
| (aa) | | |
| SARS-CoV-2 | 217 | SSYTSSNTYV |
| S2D8-v1 | | |
| mAb CDRL3 | | |
| (aa) | | |
| SARS-CoV-2 | 218 | GAGGTGCAGCTGTTGGAGTCTGGGGGAGGC |
| S2D8-v1 | | TTGGTGCAGCCTGGGGGGTCCCTGAGACTC |
| mAb VH (nt) | | TCTTGTGCAGCCTCTGGATTCACCTTTACC |
| | | ACCTATGCCATGAGTTGGGTCCGCCAGGCT |
| | | CCAGGGAAGGGGCTGGAGTGGGTCTCAGGT |
| | | ATTAGTGGTAGTGGTGGTAACACATACCA |
| | | CGCAGACTCCGTGAAGGGCCGGTTCACCAT |
| | | CTCCAGAGACAATTCCAAGAGCACGCTGTA |
| | | TCTGCAAATGAACAGCCTGAGAGCCGAGGA |
| | | CACGGCCGTGTATTACTGTGCGAAAGACCT |
| | | CTGGTTCAGGGAGATACTCCACGGTATG |
| | | GATGTCTGGGGCGAAGGGACCACGGTCAC |
| | | CGTCTCCTCAG |
| SARS-CoV-2 | 219 | CAGTCTGCCCTGACTCAGCCTGCCTCCGTGT |
| S2D8-v1 | | CTGGGTCTCCTGGACAGTCGATCACCATCT |
| mAb VL (nt) | | CCTGCACTGGAACCAGCAGTGACATTGGT |
| | | GGTTATAACTATGTCTCCTGGTACCAACAC |
| | | CACCCAGGCAAAGCCCCCAAAATAATGATT |
| | | TATGAGGTCACTAATCGGCCCTCAGGGGTT |
| | | TCTAATCGCTTCTCTGGCTCCAAGTCTGGCA |
| | | ACACGGCCTCCCTGACCATCTCTGGGCTCC |
| | | AGGCTGAGGACGAGGCTGATTATTACTGCA |
| | | GCTCATATACAAGCAGCAACACTTATGTC |
| | | TTCGGAACTGGGACCAAGGTCACCGTCCTA |
| | | G |
| SARS-CoV-2 | 220 | EVQLVESGGGLVQPGGSLRLSCAASGFPFNI |
| S2D25-v1 | | YAMSWVRQAPGKGLEWVSGISGSGGSTYYA |
| mAb VH (aa) | | DSVRGRFAISRDNSKNTLYLQMNSLRAEDTA |
| | | EYYCAKDLWFREILHGMDVWGKGTTVTVS |
| | | S |
| SARS-CoV-2 | 221 | GFPFNIYA |
| S2D25-v1 | | |
| mAb CDRH1 | | |
| (aa) | | |
| SARS-CoV-2 | 222 | ISGSGGST |
| S2D25-v1 | | |
| mAb CDRH2 | | |
| (aa) | | |
| SARS-CoV-2 | 223 | AKDLWFREILHGMDV |
| S2D25-v1 | | |
| mAb CDRH3 | | |
| (aa) | | |
| SARS-CoV-2 | 224 | QSALTQPASVSGSPGQSITISCTGTSSDVGGY |
| S2D25-v1 | | NYVSWYQHHPGRAPKLMIYEVSNRPSGVSN |
| mAb VL (aa) | | RFSGSKSGNTASLSISGLQAEDEADYYCSSYT |
| | | SSSTYVFGAGTKVTVL |
| SARS-CoV-2 | 225 | SSDVGGYNY |
| S2D25-v1 | | |
| mAb CDRL1 | | |
| (aa) | | |
| SARS-CoV-2 | 226 | EVS |
| S2D25-v1 | | |
| mAb CDRL2 | | |
| (aa) | | |
| SARS-CoV-2 | 227 | SSYTSSSTYV |
| S2D25-v1 | | |
| mAb CDRL3 | | |
| (aa) | | |
| SARS-CoV-2 | 228 | GAGGTGCAGCTGGTGGAGTCTGGGGGAGG |
| S2D25-v1 | | CTTGGTACAGCCTGGGGGGTCCCTGAGACT |
| mAb VH (nt) | | CTCCTGTGCAGCCTCTGGATTCCCATTTAA |
| | | CATCTATGCCATGAGCTGGGTCCGCCAGG |
| | | CTCCAGGGAAGGGGCTGGAGTGGGTCTCAG |
| | | GTATTAGTGGAAGTGGTGGTAGCACATAC |
| | | TACGCAGACTCCGTGAGGGGCCGGTTCGCC |
| | | ATCTCCAGAGACAATTCCAAGAACACGCTG |
| | | TATCTGCAAATGAACAGCCTGAGAGCCGAG |
| | | GACACGGCCGAATATTACTGTGCGAAAGA |
| | | CCTCTGGTTCAGGGAGATACTCCACGGT |
| | | ATGGACGTCTGGGGCAAAGGGACCACGGT |
| | | CACCGTCTCCTCAG |
| SARS-CoV-2 | 229 | CAGTCTGACCTGACTCAGCCTGCCTCCGTGT |
| S2D25-v1 | | CTGGGTCTCCTGGACAGTCGATCACCATCT |
| mAb VL (nt) | | CCTGCACTGGAACCAGCAGTGACGTTGGT |
| | | GGTTATAACTATGTCTCCTGGTACCAACAC |
| | | CACCCAGGCAGAGCCCCCAAACTCATGATT |
| | | TATGAGGTCAGTAATCGGCCCTCAGGGGTT |
| | | TCTAATCGCTTCTCTGGCTCCAAGTCTGGCA |
| | | ACACGGCCTCCCTGAGCATCTCTGGGCTCC |
| | | AGGCTGAGGACGAGGCTGATTATTACTGCA |
| | | GCTCATATACAAGCAGCAGCACTTATGTC |
| | | TTCGGAGCTGGGACCAAGGTCACCGTCCTA |
| | | G |
| SARS-CoV-2 | 230 | QSDLTQPASVSGSPGQSITISCTGTSSDVGGY |
| S2D25-v2 | | NYVSWYQHHPGRAPKLMIYEVSNRPSGVSN |
| mAb VL (aa) | | RFSGSKSGNTASLSISGLQAEDEADYYCSSYT |
| | | SSSTYVFGAGTKVTVL |
| SARS-CoV-2 | 231 | CAGTCTGCCCTGACTCAGCCTGCCTCCGTGT |
| S2D25-v2 | | CTGGGTCTCCTGGACAGTCGATCACCATCT |
| mAb VL (nt) | | CCTGCACTGGAACCAGCAGTGACGTTGGT |
| | | GGTTATAACTATGTCTCCTGGTACCAACAC |
| | | CACCCAGGCAGAGCCCCCAAACTCATGATT |
| | | TATGAGGTCAGTAATCGGCCCTCAGGGGTT |
| | | TCTAATCGCTTCTCTGGCTCCAAGTCTGGCA |
| | | ACACGGCCTCCCTGAGCATCTCTGGGCTCC |
| | | AGGCTGAGGACGAGGCTGATTATTACTGCA |
| | | GCTCATATACAAGCAGCAGCACTTATGTC |
| | | TTCGGAGCTGGGACCAAGGTCACCGTCCTA |
| | | G |
| SARS-CoV-2 | 232 | EVQLVESGGGLVQPGRSLRLSCTSSGFTFGD |
| S2D32-v1 | | YPMSWFRQAPGKGLEWVGFIRSKAYGGTTQ |
| mAb VH (aa) | | YAASVKGRFTISRDDSKNIAYLQMNSLKTED |
| | | TAVYYCTREMWDCSGGRCYSPFFDYWGQG |
| | | TLVTVSS |
| SARS-CoV-2 | 233 | GFTFGDYP |
| S2D32-v1 | | |
| mAb CDRH1 | | |
| (aa) | | |
| SARS-CoV-2 | 234 | IRSKAYGGTT |
| S2D32-v1 | | |
| mAb CDRH2 | | |
| (aa) | | |
| SARS-CoV-2 | 235 | TREMWDCSGGRCYSPFFDY |
| S2D32-v1 | | |
| mAb CDRH3 | | |
| (aa) | | |
| SARS-CoV-2 | 236 | EIVMTQSPATLSVSPGERATLSCRASQTVSSN |
| S2D32-v1 | | LAWYQQKPGQAPRLLIYGASTRATGIPARFS |
| mAb VL | | GSGSGTEFTLTISSLQSEDFAVYYCQQYNNW |
| (VK) (aa) | | RTFGQGTKLEIK |
| SARS-CoV-2 | 237 | QTVSSN |
| S2D32-v1 | | |
| mAb CDRL1 | | |
| (aa) | | |
| SARS-CoV-2 | 238 | GAS |
| S2D32-v1 | | |
| mAb CDRL2 | | |
| (aa) | | |
| SARS-CoV-2 | 239 | QQYNNWRT |
| S2D32-v1 | | |
| mAb CDRL3 | | |
| (aa) | | |
| SARS-CoV-2 | 240 | GAGGTGCAGCTGGTGGAGTCTGGGGGAGG |
| S2D32-v1 | | CTTGGTACAGCCAGGGCGGTCCCTGAGACT |
| mAb VH (nt) | | CTCCTGTACATCTTCTGGATTCACCTTTGG |
| | | TGATTATCCTATGAGCTGGTTCCGCCAGGC |
| | | TCCAGGGAAGGGGCTGGAGTGGGTAGGTTT |
| | | CATTAGAAGCAAAGCTTATGGTGGGACA |
| | | ACACAATACGCCGCGTCTGTGAAAGGCAGA |
| | | TTCACCATCTCAAGAGATGACTCCAAAAAC |
| | | ATCGCCTATCTGCAAATGAACAGCCTGAAA |
| | | ACCGAGGACACAGCCGTGTATTACTGTACT |
| | | AGAGAAATGTGGGATTGTAGTGGTGGTA |
| | | GGTGCTACTCCCCTTTTTTCGACTACTGG |
| | | GGCCAGGGAACCCTGGTCACCGTCTCCTCA |
| | | G |
| SARS-CoV-2 | 24 | GAAATAGTGATGACGCAGTCTCCAGCCACC |
| S2D32-v1 | | CTGTCTGTGTCTCCAGGGGAAAGAGCCACC |
| mAb VL(VK) | | CTCTCCTGCAGGGCCAGTCAGACTGTTAGC |
| (nt) | | AGCAACTTAGCCTGGTACCAGCAGAAACCT |
| | | GGCCAGGCTCCCAGGCTCCTCATCTATGGT |
| | | GCATCCACCAGGGCCACTGGTATCCCAGCC |
| | | AGGTTCAGTGGCAGTGGGTCTGGGACAGAG |
| | | TTCACTCTCACCATCAGCAGCCTGCAGTCTG |
| | | AAGATTTTGCAGTTTATTACTGTCAGCAGT |
| | | ATAATAACTGGCGGACTTTTGGCCAGGGG |
| | | ACCAAGCTGGAGATCAAAC |
| SARS-CoV-2 | 242 | EVQLVQSGPEVKKPGTSVKVSCKASGFTFMS |
| S2D60-v1 | | SAVQWVRQARGQRLEWIGWIVVGSGNTNYT |
| mAb VH (aa) | | QKFRERVTITRDMSTSTAYMELSSLRSEDTAV |
| | | YYCAAPRCSGGSCHDGFDIWGQGTMVTVSS |
| SARS-CoV-2 | 243 | GFTFMSSA |
| S2D60-v1 | | |
| mAb CDRH1 | | |
| (aa) | | |
| SARS-CoV-2 | 244 | IVVGSGNT |
| S2D60-v1 | | |
| mAb CDRH2 | | |
| (aa) | | |
| SARS-CoV-2 | 245 | AAPRCSGGSCHDGFDI |
| S2D60-v1 | | |
| mAb CDRH3 | | |
| (aa) | | |
| SARS-CoV-2 | 246 | EIVLTQSPGTLSLSPGERATLSCRASQSVSSSY |
| S2D60-v1 | | LGWYQQKPGQAPRLLIYGASSRATGIPDRFSG |
| mAb VL | | SGSGTDFTLTISRLEPEDFAVYYCQQYGRSP |
| (VK) (aa) | | WTFGQGTKVEIK |
| SARS-CoV-2 | 247 | QSVSSSY |
| S2D60-v1 | | |
| mAb CDRL1 | | |
| (aa) | | |
| SARS-CoV-2 | 248 | GAS |
| S2D60-v1 | | |
| mAb CDRL2 | | |
| (aa) | | |
| SARS-CoV-2 | 249 | QQYGRSPWT |
| S2D60-v1 | | |
| mAb CDRL3 | | |
| (aa) | | |
| SARS-CoV-2 | 250 | GAGGTGCAGCTGGTGCAGTCTGGGCCTGAG |
| S2D60-v1 | | GTGAAGAAGCCTGGGACCTCAGTGAAGGTC |
| mAb VH (nt) | | TCCTGCAAGGCTTCTGGATTCACCTTTATG |
| | | AGCTCTGCTGTGCAGTGGGTGCGACAGGC |
| | | TCGTGGACAACGCCTTGAGTGGATAGGATG |
| | | GATCGTCGTTGGCAGTGGTAACACAAACT |
| | | ACACACAGAAGTTCCGGGAAAGAGTCACC |
| | | ATCACCAGGGACATGTCCACAAGTACAGCC |
| | | TACATGGAGCTGAGCAGCCTGAGATCCGAG |
| | | GACACGGCCGTGTATTATTGTGCGGCTCCT |
| | | CGTTGTAGTGGTGGTAGCTGCCATGATG |
| | | GTTTTGATATCTGGGGCCAAGGGACAATG |
| | | GTCACCGTCTCTTCAG |
| SARS-CoV-2 | 251 | GAAATTGTGTTGACGCAGTCTCCAGGCACC |
| S2D60-v1 | | CTGTCTTTGTCTCCAGGGGAAAGAGCCACC |
| mAb VL(VK) | | CTCTCCTGCAGGGCCAGTCAGAGTGTTAG |
| (nt) | | CAGCAGCTACTTAGGCTGGTACCAGCAGA |
| | | AACCTGGCCAGGCTCCCAGGCTCCTCATCT |
| | | ATGGTGCATCCAGCAGGGCCACTGGCATC |
| | | CCAGACAGGTTCAGTGGCAGTGGGTCTGGG |
| | | ACAGACTTCACTCTCACCATCAGCAGACTG |
| | | GAGCCTGAAGATTTTGCAGTGTATTACTGT |
| | | CAGCAGTATGGTAGGTCACCGTGGACGT |
| | | TCGGCCAAGGGACCAAGGTGGAGATCAAA |
| | | C |
| SARS-CoV-2 | 252 | EVQLLESGGGLVQPGGSLRLSCAASGFTFTT |
| S2D8-v2 | | YAMSWVRQAPGKGLEWVSGISGSGGNTYH |
| mAb VH (aa) | | AESVKGRFTISRDNSKSTLYLQMNSLRAEDTA |
| | | VYYCAKDLWFREILHGMDVWGEGTTVTVS |
| | | S |
| SARS-CoV-2 | 253 | EVQLLESGGGLVQPGGSLRLSCAASGFTFTT |
| S2D8-v3 | | YAMSWVRQAPGKGLEWVSGISGSGGNTYH |
| mAb VH (aa) | | ADSVKGRFTISRDNSKSTLYLQMNSLRAEDT |
| | | AVYYCAKDLFFREILHGMDVWGEGTTVTVS |
| | | S |
| SARS-CoV-2 | 254 | DLFFREILHGMDV |
| S2D8-v3 | | |
| mAb CDRH3 | | |
| (aa) | | |
| SARS-CoV-2 | 255 | EVQLLESGGGLVQPGGSLRLSCAASGFTFTT |
| S2D8-v4 | | YAMSWVRQAPGKGLEWVSGISGSGGNTYH |
| mAb VH (aa) | | AESVKGRFTISRDNSKSTLYLQMNSLRAEDTA |
| | | VYYCAKDLFFREILHGMDVWGEGTTVTVSS |
| SARS-CoV-2 | 256 | EVQLLESGGGLVQPGGSLRLSCAASGFTFTT |
| S2D8-v5 | | YAMSWVRQAPGKGLEWVSGISGSGGNTYH |
| mAb VH (aa) | | ADAVKGRFTISRDNSKSTLYLQMNSLRAEDT |
| | | AVYYCAKDLWFREALHGMDVWGEGTTVT |
| | | VSS |
| SARS-CoV-2 | 257 | DLWFREALHGMDV |
| S2D8-v5 | | |
| mAb CDRH3 | | |
| (aa) | | |
| SARS-CoV-2 | 258 | EVQLLESGGGLVQPGGSLRLSCAASGFTFTT |
| S2D8-v6 | | YAMSWVRQAPGKGLEWVSGISGSGGNTYH |
| mAb VH (aa) | | ADAVKGRFTISRDNSKSTLYLQMNSLRAEDT |
| | | AVYYCAKDLFFREALHGMDVWGEGTTVTV |
| | | SS |
| SARS-CoV-2 | 259 | DLFFREALHGMDV |
| S2D8-v6 | | |
| mAb CDRH3 | | |
| (aa) | | |
| SARS-CoV-2 | 260 | EVQLLESGGGLVQPGGSLRLSCAASGFTFTT |
| S2D8-v7 | | YAMSWVRQAPGKGLEWVSGISGSGGNTYH |
| mAb VH (aa) | | ADAVKGRFTISRDNSKSTLYLQMNSLRAEDT |
| | | AVYYCAKDLYFREALHGMDVWGEGTTVTV |
| | | SS |
| SARS-CoV-2 | 261 | DLYFREALHGMDV |
| S2D8-v7 | | |
| mAb CDRH3 | | |
| (aa) | | |
| SARS-CoV-2 | 262 | EVQLVESGGGLVQPGGSLRLSCAASGFPFNI |
| S2D25-v3 | | YAMSWVRQAPGKGLEWVSGISGSGGSTYY |
| mAb VH (aa) | | AESVRGRFAISRDNSKNTLYLQMNSLRAEDT |
| | | AEYYCAKDLWFREILHGMDVWGKGTTVTV |
| | | SS |
| SARS-CoV-2 | 263 | GISGSGGSTYYAESVRG |
| S2D25-v3 | | |
| mAb CDRH2 | | |
| (aa) | | |
| SARS-CoV-2 | 264 | EVQLVESGGGLVQPGGSLRLSCAASGFPFNI |
| S2D25-v4 | | YAMSWVRQAPGKGLEWVSGISGSGGSTYY |
| mAb VH (aa) | | ADSVRGRFAISRDNSKNTLYLQMNSLRAEDT |
| | | AEYYCAKDLFFREILHGMDVWGKGTTVTVS |
| | | S |
| SARS-CoV-2 | 265 | DLFFREILHGMDV |
| S2D25-v4 | | |
| mAb CDRH3 | | |
| (aa) | | |
| SARS-CoV-2 | 266 | EVQLVESGGGLVQPGGSLRLSCAASGFPFNI |
| S2D25-v5 | | YAMSWVRQAPGKGLEWVSGISGSGGSTYY |
| mAb VH (aa) | | AESVRGRFAISRDNSKNTLYLQMNSLRAEDT |
| | | AEYYCAKDLFFREILHGMDVWGKGTTVTVS |
| | | S |
| SARS-CoV-2 | 267 | EVQLVESGGGLVQPGGSLRLSCAASGFPFNS |
| S2D25-v6 | | YAMSWVRQAPGKGLEWVSGISGSGGSTYY |
| mAb VH (aa) | | ADAVRGRFAISRDNSKNTLYLQMNSLRAEDT |
| | | AEYYCAKDLFFREILHGMDVWGKGTTVTVS |
| | | S |
| SARS-CoV-2 | 268 | GFPFNSYAMS |
| S2D25-v6 | | |
| mAb CDRH1 | | |
| (aa) | | |
| SARS-CoV-2 | 269 | GISGSGGSTYYADAVRG |
| S2D25-v6 | | |
| mAb CDRH2 | | |
| (aa) | | |
| SARS-CoV-2 | 270 | EVQLVESGGGLVQPGGSLRLSCAASGFPENS |
| S2D25-v7 | | YAMSWVRQAPGKGLEWVSGISGSGGSTYY |
| mAb VH (aa) | | ADAVRGRFAISRDNSKNTLYLQMNSLRAEDT |
| | | AEYYCAKDLWFREALHGMDVWGKGTTVT |
| | | VSS |
| SARS-CoV-2 | 271 | DLWFREALHGMDV |
| S2D25-v7 | | |
| mAb CDRH3 | | |
| (aa) | | |
| SARS-CoV-2 | 272 | EVQLVESGGGLVQPGGSLRLSCAASGFPENS |
| S2D25-v8 | | YAMSWVRQAPGKGLEWVSGISGSGGSTYY |
| mAb VH (aa) | | ADAVRGRFAISRDNSKNTLYLQMNSLRAEDT |
| | | AEYYCAKDLYFREILHGMDVWGKGTTVTV |
| | | SS |
| SARS-CoV-2 | 273 | DLYFREILHGMDV |
| S2D25-v8 | | |
| mAb CDRH3 | | |
| (aa) | | |
| SARS-CoV-2 | 274 | EVQLVESGGGLVQPGRSLRLSCTSSGFTFGD |
| S2D32-v2 | | YPMSWFRQAPGKGLEWVGFIRSKAYGGTT |
| mAb VH (aa) | | QYAASVKGRFTISRDDSKNIAYLQMNSLKTE |
| | | DTAVYYCTREMFDCSGGRCYSPFFDYWGQ |
| | | GTLVTVSS |
| SARS-CoV-2 | 275 | EMFDCSGGRCYSPFFDY |
| S2D32-v2 | | |
| mAb CDRH3 | | |
| (aa) | | |
| SARS-CoV-2 | 276 | EVQLVESGGGLVQPGRSLRLSCTSSGFTFGD |
| S2D32-v3 | | YPMSWFRQAPGKGLEWVGFIRSKAYGGTT |
| mAb VH (aa) | | QYAASVKGRFTISRDDSKNIAYLQMNSLKTE |
| | | DTAVYYCTREMWDSSGGRSYSPFFDYWGQ |
| | | GTLVTVSS |
| SARS-CoV-2 | 277 | EMWDSSGGRSYSPFFDY |
| S2D32-v3 | | |
| mAb CDRH3 | | |
| (aa) | | |
| SARS-CoV-2 | 278 | EVQLVESGGGLVQPGRSLRLSCTSSGFTFGD |
| S2D32-v4 | | YPMSWFRQAPGKGLEWVGFIRSKAYGGTT |
| mAb VH (aa) | | QYAASVKGRFTISRDDSKNIAYLQMNSLKTE |
| | | DTAVYYCTREMWDPSGGRPYSPFFDYWGQ |
| | | GTLVTVSS |
| SARS-CoV-2 | 279 | EMWDPSGGRPYSPFFDY |
| S2D32-v4 | | |
| mAb CDRH3 | | |
| (aa) | | |
| SARS-CoV-2 | 280 | EVQLVESGGGLVQPGRSLRLSCTSSGFTFGD |
| S2D32-v5 | | YPMSWFRQAPGKGLEWVGFIRSKAYGGTT |
| mAb VH (aa) | | QYAASVKGRFTISRDDSKNIAYLQMNSLKTE |
| | | DTAVYYCTREMWDASGGRAYSPFFDYWGQ |
| | | GTLVTVSS |
| SARS-CoV-2 | 281 | EMWDASGGRAYSPFFDY |
| S2D32-v5 | | |
| mAb CDRH3 | | |
| (aa) | | |
| SARS-CoV-2 | 282 | EIVMTQSPATLSVSPGERATLSCRASQTVSSN |
| S2D32-v6 | | LAWYQQKPGQAPRLLIYGASTRATGIPARFS |
| mAb VL(VK) | | GSGSGTEFTLTISSLQSEDFAVYYCQQYNNFR |
| (aa) | | TFGQGTKLEIK |
| SARS-CoV-2 | 283 | QQYNNFRT |
| S2D32-v6 | | |
| mAb CDRL3 | | |
| (aa) | | |
| SARS-CoV-2 | 284 | EVQLVQSGPEVKKPGTSVKVSCKASGFTFMS |
| S2D60-v2 | | SAVQWVRQARGQRLEWIGFIVVGSGNTNYA |
| mAb VH (aa) | | QKFRERVTITRDMSTSTAYMELSSLRSEDTA |
| | | VYYCAAPRCSGGSCHDGFDIWGQGTMVTV |
| | | SS |
| SARS-CoV-2 | 285 | FIVVGSGNTNYAQKFRE |
| S2D60-v2 | | |
| mAb CDRH2 | | |
| (aa) | | |
| SARS-CoV-2 | 286 | EVQLVQSGPEVKKPGTSVKVSCKASGFTFMS |
| S2D60-v3 | | SAVQWVRQARGQRLEWIGFIVVGSGNTQYT |
| mAb VH (aa) | | QKFRERVTITRDMSTSTAYMELSSLRSEDTA |
| | | VYYCAAPRCSGGSCHDGFDIWGQGTMVTV |
| | | SS |
| SARS-CoV-2 | 287 | FIVVGSGNTQYTQKFRE |
| S2D60-v3 | | |
| mAb CDRH2 | | |
| (aa) | | |
| SARS-CoV-2 | 288 | EVQLVQSGPEVKKPGTSVKVSCKASGFTFMS |
| | | SAVQWVRQARGQRLEWIGWIVVGSGNTNY |
| S2D60-v4 | | AQKFRERVTITRDMSTSTAYMELSSLRSEDT |
| mAb VH (aa) | | AVYYCAAPRCSGGSCHDAFDIWGQGTMVT |
| | | VSS |
| SARS-CoV-2 | 289 | WIVVGSGNTNYAQKFRE |
| S2D60-v4 | | |
| mAb CDRH2 | | |
| (aa) | | |
| SARS-CoV-2 | 290 | PRCSGGSCHDAFDI |
| S2D60-v4 | | |
| mAb CDRH3 | | |
| (aa) | | |
| SARS-CoV-2 | 291 | EVQLVQSGPEVKKPGTSVKVSCKASGFTFMS |
| S2D60-v5 | | SAVQWVRQARGQRLEWIGFIVVGSGNTNYA |
| mAb VH (aa) | | QKFRERVTITRDMSTSTAYMELSSLRSEDTA |
| | | VYYCAAPRCSGGSCHDAFDIWGQGTMVTV |
| | | SS |
| SARS-CoV-2 | 292 | EVQLVQSGPEVKKPGTSVKVSCKASGFTFMS |
| S2D60-v6 | | SAVQWVRQARGQRLEWIGYIVVGSGNTQYT |
| mAb VH (aa) | | QKFRERVTITRDMSTSTAYMELSSLRSEDTA |
| | | VYYCAAPRCSGGSCHEGFDIWGQGTMVTV |
| | | SS |
| SARS-CoV-2 | 293 | YIVVGSGNTQYTQKFRE |
| S2D60-v6 | | |
| mAb CDRH2 | | |
| (aa) | | |
| SARS-CoV-2 | 294 | PRCSGGSCHEGFDI |
| S2D60-v6 | | |
| mAb CDRH3 | | |
| (aa) | | |
| SARS-CoV-2 | 295 | EVQLVQSGPEVKKPGTSVKVSCKASGFTFMS |
| S2D60-v7 | | SAVQWVRQARGQRLEWIGWIVVGSGNTNY |
| mAb VH (aa) | | AQKFRERVTITRDMSTSTAYMELSSLRSEDT |
| | | AVYYCAAPRSSGGSSHDGFDIWGQGTMVTV |
| | | SS |
| SARS-CoV-2 | 296 | PRSSGGSSHDGFDI |
| S2D60-v7 | | |
| mAb CDRH3 | | |
| (aa) | | |
| SARS-CoV-2 | 297 | EVQLVQSGPEVKKPGTSVKVSCKASGFTFMS |
| S2D60-v8 | | SAVQWVRQARGQRLEWIGWIVVGSGNTNY |
| mAb VH (aa) | | AQKFRERVTITRDMSTSTAYMELSSLRSEDT |
| | | AVYYCAAPRCSGGSCHDGFDIWGQGTMVT |
| | | VSS |
| SARS-CoV-2 | 298 | EVQLVQSGPEVKKPGTSVKVSCKASGFTFMS |
| S2D60-v9 | | SAVQWVRQARGQRLEWIGWIVVGSGNTQY |
| mAb VH (aa) | | TQKFRERVTITRDMSTSTAYMELSSLRSEDT |
| | | AVYYCAAPRCSGGSCHDGFDIWGQGTMVT |
| | | VSS |
| SARS-CoV-2 | 299 | WIVVGSGNTQYTQKFRE |
| S2D60-v9 | | |
| mAb CDRH2 | | |
| (aa) | | |
| SARS-CoV-2 | 300 | EVQLVQSGPEVKKPGTSVKVSCKASGFTFMS |
| S2D60-v10 | | SAVQWVRQARGQRLEWIGWIVVGSGNTDY |
| mAb VH (aa) | | TQKFRERVTITRDMSTSTAYMELSSLRSEDT |
| | | AVYYCAAPRCSGGSCHDGFDIWGQGTMVT |
| | | VSS |
| SARS-CoV-2 | 301 | WIVVGSGNTDYTQKFRE |
| S2D60-v10 | | |
| mAb CDRH2 | | |
| (aa) | | |
| SARS-CoV-2 | 302 | EIVLTQSPGTLSLSPGERATLSCRASQSVSSSY |
| S2D60-v11 | | LGWYQQKPGQAPRLLIYGASSRATGIPDRFS |
| mAb VL(VK) | | GSGSGTDFTLTISRLEPEDFAVYYCQQYGRSP |
| (aa) | | FTFGQGTKVEIK |
| SARS-CoV-2 | 303 | QQYGRSPFT |
| S2D60-v11 | | |
| mAb CDRL3 | | |
| (aa) | | |
| SARS-CoV-2 | 304 | EVHLVESGGGLVQPGGSLKLSCAASGFTFSG |
| S2D22-v1 | | SAMHWVRQASGKGLEWVGRIRTKANTYAT |
| mAb VH (aa) | | AYAASVKGRFTISRDDSKNTAYLQMNSLKTE |
| | | DTAVYYCTRPAPYDFLSDYYTGEQLDYWG |
| | | QGTLVTVSS |
| SARS-CoV-2 | 305 | GFTFSGSA |
| S2D22-v1 | | |
| mAb CDRH1 | | |
| (aa) | | |
| SARS-CoV-2 | 306 | IRTKANTYAT |
| S2D22-v1 | | |
| mAb CDRH2 | | |
| (aa) | | |
| SARS-CoV-2 | 307 | TRPAPYDFLSDYYTGEQLDY |
| S2D22-v1 | | |
| mAb CDRH3 | | |
| (aa) | | |
| SARS-CoV-2 | 308 | QSVLTQPPSVSGAPGQRVTISCTGSSSNIGAG |
| S2D22-v1 | | YDVHWYQQLPGAAPKLLIYGNSNRPSGVPDR |
| mAb VL (aa) | | FSGSKSGTSASLAITGLQAEDEADYYCQSYDS |
| | | SLSGPEVFGTGTKVTVL |
| SARS-CoV-2 | 309 | SSNIGAGYD |
| S2D22-v1 | | |
| mAb CDRL1 | | |
| (aa) | | |
| SARS-CoV-2 | 310 | GNS |
| S2D22-v1 | | |
| mAb CDRL2 | | |
| (aa) | | |
| SARS-CoV-2 | 311 | QSYDSSLSGPEV |
| S2D22-v1 | | |
| mAb CDRL3 | | |
| (aa) | | |
| SARS-CoV-2 | 312 | GAGGTGCATCTGGTGGAGTCCGGGGGAGGC |
| S2D22-v1 | | TTGGTCCAGCCTGGGGGGTCCCTGAAACTC |
| mAb VH (nt) | | TCCTGTGCAGCCTCTGGGTTCACCTTCAGT |
| | | GGCTCTGCTATGCACTGGGTCCGCCAGGCT |
| | | TCCGGGAAAGGGCTGGAGTGGGTTGGCCGT |
| | | ATAAGAACCAAAGCTAATACTTACGCGA |
| | | CAGCATATGCTGCGTCGGTGAAAGGCAGGT |
| | | TCACCATCTCCAGAGATGATTCAAAGAACA |
| | | CGGCTTATCTGCAAATGAACAGCCTGAAAA |
| | | CCGAGGACACGGCCGTGTATTACTGTACTA |
| | | GACCCGCCCCCTACGATTTTTTGAGTGAT |
| | | TATTATACCGGTGAACAACTTGACTACTG |
| | | GGGCCAGGGAACCCTGGTCACCGTCTCCTC |
| | | AG |
| SARS-CoV-2 | 313 | CAGTCTGTGCTGACGCAGCCGCCCTCAGTG |
| S2D22-v1 | | TCTGGGGCCCCAGGGCAGAGGGTCACCATC |
| mAb VL (nt) | | TCCTGCACTGGGAGCAGCTCCAACATCGG |
| | | GGCAGGTTATGATGTACACTGGTACCAGC |
| | | AACTTCCAGGAGCAGCCCCCAAACTCCTCA |
| | | TCTATGGTAACAGCAATCGGCCCTCAGGG |
| | | GTCCCTGACCGATTCTCTGGCTCCAAGTCTG |
| | | GCACCTCAGCCTCCCTGGCCATCACTGGGC |
| | | TCCAGGCTGAGGATGAGGCTGATTATTACT |
| | | GCCAGTCCTATGACAGCAGCCTGAGTGG |
| | | TCCGGAGGTCTTCGGAACTGGGACCAAGG |
| | | TCACCGTCCTGG |
| SARS-CoV-2 | 314 | QVTLKESGPTLVKPTQTLTLTCTFSGFSLSTS |
| S2D43-v1 | | GVGVGWLRQPPGKALEWLALIYWDDDKRY |
| mAb VH (aa) | | SPSLKNRLTVTKDTSKNQVVLTMTNLDPVDT |
| | | ATYYCAHDNTLGGSSTWQSTFDYWGQGTL |
| | | VTVSS |
| SARS-CoV-2 | 315 | GFSLSTSGVG |
| S2D43-v1 | | |
| mAb CDRH1 | | |
| (aa) | | |
| SARS-CoV-2 | 316 | IYWDDDK |
| S2D43-v1 | | |
| mAb CDRH2 | | |
| (aa) | | |
| SARS-CoV-2 | 317 | AHDNTLGGSSTWQSTEDY |
| S2D43-v1 | | |
| mAb CDRH3 | | |
| (aa) | | |
| SARS-CoV-2 | 318 | QSVLTQPPSVSEAPRQRVTISCSGSNSNIGNN |
| S2D43-v1 | | AVHWYQQLPGKAPKLLIYYDDLLPSGVSDRF |
| mAb VL (aa) | | SGSKSGTSASLAISGLQSEDEADYYCAAWDD |
| | | RMNGPVFGGGTKLTVL |
| SARS-CoV-2 | 319 | NSNIGNNA |
| S2D43-v1 | | |
| mAb CDRL1 | | |
| (aa) | | |
| SARS-CoV-2 | 320 | YDD |
| S2D43-v1 | | |
| mAb CDRL2 | | |
| (aa) | | |
| SARS-CoV-2 | 321 | AAWDDRMNGPV |
| S2D43-v1 | | |
| mAb CDRL3 | | |
| (aa) | | |
| SARS-CoV-2 | 322 | CAGGTCACCTTGAAGGAGTCTGGTCCTACG |
| S2D43-v1 | | CTGGTGAAACCCACACAGACCCTCACGCTG |
| mAb VH (nt) | | ACCTGCACCTTCTCTGGGTTCTCACTCAGC |
| | | ACTAGTGGAGTGGGTGTGGGCTGGCTCCG |
| | | TCAGCCCCCAGGAAAGGCCCTGGAGTGGCT |
| | | TGCACTCATTTATTGGGATGATGATAAGC |
| | | GCTACAGTCCATCTCTGAAGAATAGGCTCA |
| | | CCGTCACCAAGGACACCTCCAAAAACCAGG |
| | | TGGTCCTCACAATGACCAATTTGGACCCTG |
| | | TGGACACAGCCACATATTACTGTGCACACG |
| | | ACAACACCTTGGGAGGTAGCAGCACCTG |
| | | GCAATCAACCTTTGACTACTGGGGCCAGG |
| | | GAACCCTGGTCACCGTCTCCTCAG |
| SARS-CoV-2 | 323 | CAGTCTGTGCTGACTCAGCCACCCTCGGTG |
| S2D43-v1 | | TCTGAAGCCCCCAGGCAGAGGGTCACCATC |
| mAb VL (nt) | | TCCTGTTCTGGAAGCAACTCCAACATCGG |
| | | AAATAATGCTGTACACTGGTACCAGCAGCT |
| | | CCCAGGAAAGGCTCCCAAACTCCTCATCTA |
| | | TTATGATGATCTGCTGCCCTCAGGGGTCTC |
| | | TGACCGATTCTCTGGCTCCAAGTCTGGCAC |
| | | CTCAGCCTCCCTGGCCATCAGTGGGCTCCA |
| | | GTCTGAGGATGAGGCTGATTATTACTGTGC |
| | | AGCATGGGATGACAGGATGAATGGTCCG |
| | | GTATTCGGCGGAGGGACCAAGCTGACCGTC |
| | | CTAG |
| SARS-CoV-2 | 324 | DLWFREILHGMDV |
| S2D8-v1.1 | | |
| mAb CDRH3 | | |
| (aa) | | |
| SARS-CoV-2 | 325 | GFPFNIYAMS |
| S2D25-v1.1 | | |
| mAb CDRH1 | | |
| (aa) | | |
| SARS-CoV-2 | 326 | GISGSGGSTYYADSVRG |
| S2D25-v1.2 | | |
| mAb CDRH2 | | |
| (aa) | | |
| SARS-CoV-2 | 327 | DLWFREILHGMDV |
| S2D25-v1.3 | | |
| mAb CDRH3 | | |
| (aa) | | |
| SARS-CoV-2 | 328 | TGTSSDVGGYNYVS |
| S2D25-v1.4 | | |
| mAb CDRL1 | | |
| (aa) | | |
| SARS-CoV-2 | 329 | EVSNRPS |
| S2D25-v1.5 | | |
| mAb CDRL2 | | |
| (aa) | | |
| SARS-CoV-2 | 330 | GFTFGDYPMS |
| S2D32-v1.1 | | |
| mAb CDRH1 | | |
| (aa) | | |
| SARS-CoV-2 | 331 | FIRSKAYGGTTQYAASVKG |
| S2D32-v1.2 | | |
| mAb CDRH2 | | |
| (aa) | | |
| SARS-CoV-2 | 332 | EMWDCSGGRCYSPFFDY |
| S2D32-v1.3 | | |
| mAb CDRH3 | | |
| (aa) | | |
| SARS-CoV-2 | 333 | RASQTVSSNLA |
| S2D32-v1.4 | | |
| mAb CDRL1 | | |
| (aa) | | |
| SARS-CoV-2 | 334 | GASTRAT |
| S2D32-v1.5 | | |
| mAb CDRL2 | | |
| (aa) | | |
| SARS-CoV-2 | 335 | GFTFMSSAVQ |
| S2D60-v1.1 | | |
| mAb CDRH1 | | |
| (aa) | | |
| SARS-CoV-2 | 336 | WIVVGSGNTNYTQKFRE |
| S2D60-v1.2 | | |
| mAb CDRH2 | | |
| (aa) | | |
| SARS-CoV-2 | 337 | PRCSGGSCHDGFDI |
| S2D60-v1.3 | | |
| mAb CDRH3 | | |
| (aa) | | |
| SARS-CoV-2 | 338 | RASQSVSSSYLG |
| S2D60-v1.4 | | |
| mAb CDRL1 | | |
| (aa) | | |
| SARS-CoV-2 | 339 | GASSRAT |
| S2D60-v1.5 | | |
| mAb CDRL2 | | |
| (aa) | | |
| SARS-CoV-2 | 340 | QVQLVQSGAEVKKPGASVKVSCKASGYPFT |
| S309-v2 mAb | | SYGISWVRQAPGQGLEWMGWISTYQGNTN |
| VH (aa) | | YAQKFQGRVTMTTDTSTTTGYMELRRLRSD |
| | | DTAVYYCARDYTRGAWFGESLIGGFDNWG |
| | | QGTLVTVSS |
| SARS-CoV-2 | 341 | GYPFTSYG |
| S309-v2 mAb | | |
| CDRH1 (aa) | | |
| SARS-CoV-2 | 342 | ISTYQGNT |
| S309-v2 mAb | | |
| CDRH2 (aa) | | |
| SARS-CoV-2 | 343 | ARDYTRGAWFGESLIGGFDN |
| S309-v2 mAb | | |
| CDRH3 (aa) | | |
| SARS-CoV-2 | 344 | EIVLTQSPGTLSLSPGERATLSCRASQTVSSTS |
| S309-v2 mAb | | LAWYQQKPGQAPRLLIYGASSRATGIPDRFSG |
| VL(VK) (aa) | | SGSGTDFTLTISRLEPEDFAVYYCQQHDTSLT |
| | | FGGGTKVEIK |
| SARS-CoV-2 | 345 | QTVSSTS |
| S309-v2 mAb | | |
| CDRL1 (aa) | | |
| SARS-CoV-2 | 346 | GAS |
| S309-v2 mAb | | |
| CDRL2 (aa) | | |
| SARS-CoV-2 | 347 | QQHDTSLT |
| S309-v2 mAb | | |
| CDRL3 (aa) | | |
| SARS-CoV-2 | 348 | EVQLVESGGGLIQPGGSLRLSCAASGLTVRS |
| S2X127-v1 | | NYMSWVRQAPGKGLEWVSVMYSGGSTFYA |
| mAb VH (aa) | | DSVKGRSTISRDNSKNTLYLQMNSLRAEDTA |
| | | VYYCARGDIADDYHYGLDVWGQGTTVTVS |
| | | S |
| SARS-CoV-2 | 349 | GLTVRSNY |
| S2X127-v1 | | |
| mAb CDRH1 | | |
| (aa) | | |
| SARS-CoV-2 | 350 | MYSGGST |
| S2X127-v1 | | |
| mAb CDRH2 | | |
| (aa) | | |
| SARS-CoV-2 | 351 | ARGDIADDYHYGLDV |
| S2X127-v1 | | |
| mAb CDRH3 | | |
| (aa) | | |
| SARS-CoV-2 | 352 | QSVLTQPPSVSGAPGQRVTISCTGSTSNIGAG |
| S2X127-v1 | | YNVHWYQHFPGTSPKLLIYGNSNRPSGVPDR |
| mAb VL (aa) | | FSGSKSGTSASLAITGLQAEDEAAYYCQSYDS |
| | | NLSGVFGGGTKLTVL |
| SARS-CoV-2 | 353 | TSNIGAGYN |
| S2X127-v1 | | |
| mAb CDRL1 | | |
| (aa) | | |
| SARS-CoV-2 | 354 | GNS |
| S2X127-v1 | | |
| mAb CDRL2 | | |
| (aa) | | |
| SARS-CoV-2 | 355 | QSYDSNLSGV |
| S2X127-v1 | | |
| mAb CDRL3 | | |
| (aa) | | |
| SARS-CoV-2 | 356 | GAGGTGCAGCTGGTGGAGTCTGGAGGAGG |
| S2X127-v1 | | CTTGATCCAGCCGGGGGGGTCCCTGAGACT |
| mAb VH (nt) | | CTCCTGTGCAGCCTCTGGGCTCACCGTCA |
| | | GGAGCAACTATATGAGCTGGGTCCGCCAG |
| | | GCTCCAGGGAAGGGGCTGGAGTGGGTCTCA |
| | | GTTATGTATAGCGGTGGTAGTACATTCTA |
| | | CGCAGACTCCGTGAAGGGCCGATCCACCAT |
| | | CTCCAGAGACAATTCCAAGAACACGCTGTA |
| | | TCTTCAAATGAACAGCCTGAGAGCCGAGGA |
| | | CACGGCCGTGTATTACTGTGCGAGAGGTG |
| | | ATATAGCAGATGACTACCACTACGGTTTG |
| | | GACGTCTGGGGCCAAGGGACCACGGTCAC |
| | | CGTCTCCTCG |
| SARS-CoV-2 | 357 | CAGTCTGTGTTGACGCAGCCGCCCTCAGTG |
| S2X127-v1 | | TCTGGGGCCCCAGGGCAGAGGGTCACCATC |
| mAb VL (nt) | | TCCTGCACTGGGAGCACCTCCAACATCGG |
| | | GGCAGGTTATAATGTACACTGGTACCAGC |
| | | ACTTTCCAGGAACATCCCCCAAACTCCTCA |
| | | TCTATGGTAACAGCAATCGGCCCTCAGGG |
| | | GTCCCTGACCGATTTTCTGGCTCCAAGTCTG |
| | | GCACCTCAGCCTCCCTGGCCATCACTGGGC |
| | | TCCAGGCTGAGGATGAGGCTGCTTATTACT |
| | | GCCAGTCCTATGACAGCAACCTGAGTGG |
| | | AGTGTTCGGCGGAGGGACCAAGCTGACCG |
| | | TCCTAG |
| SARS-CoV-2 | 358 | EVQLVQSGAEVKKPGASVKVSCKASGYTFT |
| S2X129-v1 | | DYFMHWVRQAPGQGLEWMGWISPNSGGTN |
| mAb VH (aa) | | YAQRFQGRVTMTRDTSISTTYMELSRLRSDD |
| | | TAVYYCARDQAYIVLAQGSGMDVWGQGTT |
| | | VTVSS |
| SARS-CoV-2 | 359 | GYTFTDYF |
| S2X129-v1 | | |
| mAb CDRH1 | | |
| (aa) | | |
| SARS-CoV-2 | 360 | ISPNSGGT |
| S2X129-v1 | | |
| mAb CDRH2 | | |
| (aa) | | |
| SARS-CoV-2 | 361 | ARDQAYIVLAQGSGMDV |
| S2X129-v1 | | |
| mAb CDRH3 | | |
| (aa) | | |
| SARS-CoV-2 | 362 | DIVMTQTPSSVSASVGDRVTITCRASQGISSW |
| S2X129-v1 | | LAWYQQKPGKAPKLLISAASSLQSGVPSRFSG |
| mAb VL(VK) | | SGSGTDFTLTISTLQPEDFATYYCQQANSFPL |
| (aa) | | AFGPGTKVDIK |
| SARS-CoV-2 | 363 | QGISSW |
| S2X129-v1 | | |
| mAb CDRL1 | | |
| (aa) | | |
| SARS-CoV-2 | 364 | AAS |
| S2X129-v1 | | |
| mAb CDRL2 | | |
| (aa) | | |
| SARS-CoV-2 | 365 | QQANSFPLA |
| S2X129-v1 | | |
| mAb CDRL3 | | |
| (aa) | | |
| SARS-CoV-2 | 366 | GAGGTGCAGCTGGTGCAGTCTGGGGCTGAG |
| S2X129-v1 | | GTGAAGAAGCCTGGGGCCTCCGTGAAGGTC |
| mAb VH (nt) | | TCCTGCAAGGCTTCTGGATACACCTTCACC |
| | | GACTACTTTATGCACTGGGTGCGACAGGCC |
| | | CCTGGACAAGGGCTTGAGTGGATGGGATGG |
| | | ATCAGCCCTAACAGTGGTGGCACAAACT |
| | | ATGCACAGAGGTTTCAGGGCAGGGTCACCA |
| | | TGACCAGGGACACGTCCATCAGTACAACCT |
| | | ACATGGAGCTGAGCAGGCTGAGATCTGACG |
| | | ACACGGCCGTGTATTACTGTGCGAGAGAT |
| | | CAGGCGTATATTGTTCTAGCCCAAGGCT |
| | | CCGGTATGGACGTCTGGGGCCAAGGGACC |
| | | ACGGTCACCGTCTCCTCA |
| SARS-CoV-2 | 367 | GATATTGTGATGACCCAGACTCCATCTTCC |
| S2X129-v1 | | GTGTCTGCATCTGTAGGAGACAGAGTCACC |
| mAb VL(VK) | | ATCACTTGTCGGGCGAGTCAGGGTATTAG |
| (nt) | | CAGCTGGTTAGCCTGGTATCAGCAGAAAC |
| | | CAGGGAAAGCCCCTAAGCTCCTGATCTCTG |
| | | CTGCATCCAGTTTGCAAAGTGGGGTCCCAT |
| | | CAAGGTTCAGCGGCAGTGGATCTGGGACAG |
| | | ATTTCACTCTCACCATCAGCACCCTGCAGCC |
| | | TGAAGATTTTGCAACTTACTATTGTCAACA |
| | | GGCTAACAGTTTCCCCCTCGCTTTCGGCC |
| | | CTGGGACCAAAGTGGATATCAAAC |
| SARS-CoV-2 | 368 | EVQLVESGGGLVQPGGSLRLSCAASGFTVSS |
| S2X132-v1 | | NYMSWVRQAPGKGLEWVSVIYSGGNTYYA |
| mAb VH (aa) | | DSVKGRFTISRDNSKNTLYLQMISLRAEDTAV |
| | | YYCARDRRLPSIIFGLDVWGQGTTVTVSS |
| SARS-CoV-2 | 369 | GFTVSSNY |
| S2X132-v1 | | |
| mAb CDRH1 | | |
| (aa) | | |
| SARS-CoV-2 | 370 | IYSGGNT |
| S2X132-v1 | | |
| mAb CDRH2 | | |
| (aa) | | |
| SARS-CoV-2 | 371 | ARDRRLPSIIFGLDV |
| S2X132-v1 | | |
| mAb CDRH3 | | |
| (aa) | | |
| SARS-CoV-2 | 372 | QSALTQPASVSGSPGQSITISCTGTSSDVGGN |
| S2X132-v1 | | NHVSWYRQHPGKAPKLMIYEVSNRPSGVSN |
| mAb VL (aa) | | RFSGSKSGNTASLTISGLQAGDEADYYCSSFT |
| | | TYTTDVVFGGGTKLTVL |
| SARS-CoV-2 | 373 | SSDVGGNNH |
| S2X132-v1 | | |
| mAb CDRL1 | | |
| (aa) | | |
| SARS-CoV-2 | 374 | EVS |
| S2X132-v1 | | |
| mAb CDRL2 | | |
| (aa) | | |
| SARS-CoV-2 | 375 | SSFTTYTTDVV |
| S2X132-v1 | | |
| mAb CDRL3 | | |
| (aa) | | |
| SARS-CoV-2 | 376 | GAGGTGCAGCTGGTGGAGTCTGGAGGAGG |
| S2X132-v1 | | CTTGGTCCAGCCTGGGGGGTCCCTGAGACT |
| mAb VH (nt) | | CTCCTGTGCAGCCTCTGGATTCACCGTCAG |
| | | TAGCAACTACATGAGCTGGGTCCGCCAGG |
| | | CTCCAGGGAAGGGGCTGGAGTGGGTCTCAG |
| | | TTATTTATAGCGGTGGTAACACATACTAC |
| | | GCAGACTCCGTAAAGGGCCGATTCACCATC |
| | | TCCAGAGACAATTCCAAGAACACGCTGTAT |
| | | CTTCAAATGATCAGCCTGAGAGCCGAGGAC |
| | | ACGGCCGTGTATTACTGTGCGAGAGATAG |
| | | GAGGTTGCCCTCGATCATCTTCGGTCTG |
| | | GACGTCTGGGGCCAAGGGACCACGGTCAC |
| | | CGTCTCCTCA |
| SARS-CoV-2 | 377 | CAGTCTGCCCTGACTCAGCCTGCCTCCGTGT |
| S2X132-v1 | | CTGGGTCTCCTGGACAGTCGATCACCATCT |
| mAb VL (nt) | | CCTGCACTGGAACCAGCAGTGACGTTGGT |
| | | GGTAATAACCATGTCTCCTGGTACCGACAG |
| | | CACCCAGGCAAAGCCCCCAAACTCATGATT |
| | | TATGAGGTCAGTAATCGGCCCTCAGGGGTT |
| | | TCTAATCGCTTCTCTGGCTCCAAGTCTGGCA |
| | | ACACGGCCTCCCTGACCATCTCTGGGCTCC |
| | | AGGCTGGGGACGAGGCTGATTATTACTGCA |
| | | GCTCATTTACAACCTACACCACGGATGTG |
| | | GTATTCGGCGGAGGGACCAAGCTTACCGTC |
| | | CTAG |
| SARS-CoV-2 | 378 | EVQLVESGGGVVQPGRSLRLSCAASGFPFST |
| S2X190-v1 | | YGMHWVRQAPGKGLEWVAVIWYDGSTKY |
| mAb VH (aa) | | YADSVKGRFTISRDNSKNTLYLQMNSLRAED |
| | | TALYYCARVHSSGPGDEYFQHWGQGTLLTV |
| | | SS |
| SARS-CoV-2 | 379 | GFPFSTYG |
| S2X190-v1 | | |
| mAb CDRH1 | | |
| (aa) | | |
| SARS-CoV-2 | 380 | IWYDGSTK |
| S2X190-v1 | | |
| mAb CDRH2 | | |
| (aa) | | |
| SARS-CoV-2 | 381 | ARVHSSGPGDEYFQH |
| S2X190-v1 | | |
| mAb CDRH3 | | |
| (aa) | | |
| SARS-CoV-2 | 382 | DIVMTQSPSSLSASVGDRVTITCRASQGISSGL |
| S2X190-v1 | | AWYQQKPGKGPKVLIYDASSLESGVPSRFSG |
| mAb VL(VK) | | SASGTDFTLTISSLQPEDFATYFCQQFYSYPL |
| (aa) | | TFGGGTKVEIK |
| SARS-CoV-2 | 383 | QGISSG |
| S2X190-v1 | | |
| mAb CDRL1 | | |
| (aa) | | |
| SARS-CoV-2 | 384 | DAS |
| S2X190-v1 | | |
| mAb CDRL2 | | |
| (aa) | | |
| SARS-CoV-2 | 385 | QQFYSYPLT |
| S2X190-v1 | | |
| mAb CDRL3 | | |
| (aa) | | |
| SARS-CoV-2 | 386 | GAGGTGCAGCTGGTGGAGTCTGGGGGAGG |
| S2X190-v1 | | CGTGGTCCAGCCTGGCAGGTCCCTGAGACT |
| mAb VH (nt) | | CTCCTGTGCAGCGTCTGGATTCCCCTTCAG |
| | | TACCTATGGCATGCACTGGGTCCGCCAGG |
| | | CTCCAGGCAAGGGGCTGGAGTGGGTGGCA |
| | | GTTATATGGTATGATGGAAGTACTAAATA |
| | | CTATGCAGACTCCGTGAAGGGCCGATTCAC |
| | | CATCTCCAGAGACAATTCCAAGAACACTCT |
| | | CTATCTGCAAATGAACAGCCTCAGAGCCGA |
| | | GGACACGGCTTTGTATTACTGTGCGAGAGT |
| | | CCATAGCAGTGGCCCGGGGGATGAATAC |
| | | TTCCAGCACTGGGGCCAGGGCACCCTGCTC |
| | | ACCGTCTCCTCAG |
| SARS-CoV-2 | 387 | GACATCGTGATGACCCAGTCTCCATCCTCC |
| S2X190-v1 | | CTGTCTGCATCTGTAGGAGACAGAGTCACC |
| mAb VL(VK) | | ATCACTTGCCGGGCAAGTCAGGGCATCAG |
| (nt) | | CAGTGGTTTAGCCTGGTATCAACAGAAACC |
| | | AGGGAAAGGTCCTAAGGTCCTGATCTATGA |
| | | TGCCTCCAGTTTGGAAAGTGGAGTCCCATC |
| | | AAGGTTCAGCGGCAGTGCATCTGGGACAGA |
| | | TTTCACTCTCACCATCAGCAGCCTGCAGCCT |
| | | GAAGATTTTGCAACTTATTTCTGTCAACAG |
| | | TTTTATAGTTACCCTCTCACTTTCGGCGGA |
| | | GGGACCAAGGTGGAGATCAAGC |
| SARS-CoV-2 | 388 | EVHLVESGGGLIQPGGSLRLSCAASGITVSSN |
| S2X200-v1 | | YMSWVRQAPGKGLEWVSVIYSGGSTFYADS |
| mAb VH (aa) | | VKGRFTISRDNSKNTVYLQMNSLRAEDTAVY |
| | | YCARDLVTYGMDVWGQGTTVTVSS |
| SARS-CoV-2 | 389 | GITVSSNY |
| S2X200-v1 | | |
| mAb CDRH1 | | |
| (aa) | | |
| SARS-CoV-2 | 390 | IYSGGST |
| S2X200-v1 | | |
| mAb CDRH2 | | |
| (aa) | | |
| SARS-CoV-2 | 391 | ARDLVTYGMDV |
| S2X200-v1 | | |
| mAb CDRH3 | | |
| (aa) | | |
| SARS-CoV-2 | 392 | EIVLTQSPSFLSASVGDRVTITCRASQGISSYL |
| S2X200-v1 | | AWYQQKPGKAPKLLIYAASTLQSGVPSRFSG |
| mAb VL(VK) | | SGSGTEFTLTISSLQPEDFATYYCQQLNGDPP |
| (aa) | | IFGHGTRLEIK |
| SARS-CoV-2 | 393 | QGISSY |
| S2X200-v1 | | |
| mAb CDRL1 | | |
| (aa) | | |
| SARS-CoV-2 | 394 | AAS |
| S2X200-v1 | | |
| mAb CDRL2 | | |
| (aa) | | |
| SARS-CoV-2 | 395 | QQLNGDPPI |
| S2X200-v1 | | |
| mAb CDRL3 | | |
| (aa) | | |
| SARS-CoV-2 | 396 | GAGGTGCATCTGGTGGAGTCTGGAGGAGGC |
| S2X200-v1 | | TTGATCCAGCCTGGGGGGTCCCTGAGACTC |
| mAb VH (nt) | | TCCTGTGCAGCCTCTGGGATCACCGTCAG |
| | | TAGCAACTATATGAGCTGGGTCCGCCAGG |
| | | CTCCAGGGAAGGGGCTGGAGTGGGTCTCAG |
| | | TTATTTATAGCGGTGGTAGCACATTCTAT |
| | | GCAGACTCCGTGAAGGGCCGATTCACCATC |
| | | TCCAGAGACAATTCCAAGAACACGGTGTAT |
| | | CTTCAAATGAACAGCCTGAGAGCCGAGGAC |
| | | ACGGCCGTGTATTACTGTGCGAGAGATCT |
| | | GGTAACCTACGGTATGGACGTCTGGGGCC |
| | | AAGGGACCACGGTCACCGTCTCCTCA |
| SARS-CoV-2 | 397 | GAAATTGTGTTGACACAGTCTCCATCCTTCC |
| S2X200-v1 | | TGTCTGCATCTGTAGGAGACAGAGTCACCA |
| mAb VL(VK) | | TCACTTGCCGGGCCAGTCAGGGCATTAGC |
| (nt) | | AGTTATTTAGCCTGGTATCAGCAAAAACCA |
| | | GGGAAAGCCCCTAAGCTCCTGATCTATGCT |
| | | GCATCCACTTTGCAAAGTGGGGTCCCATCA |
| | | AGGTTCAGCGGCAGTGGATCTGGGACAGAA |
| | | TTCACTCTCACAATCAGCAGCCTGCAGCCT |
| | | GAAGATTTTGCAACTTATTACTGTCAACAG |
| | | CTTAATGGTGACCCTCCTATCTTCGGCCA |
| | | TGGGACACGACTGGAGATTAAAC |
| SARS-CoV-2 | 398 | EVQLVQSGAEVKKPGASVKVSCKASGYTFTS |
| S2X227-v1 | | YYMHWVRQAPGQGLEWMGIINPGGVSTTY |
| mAb VH (aa) | | AHYAQKFQGRVTMTRDTSTSTVYMELSSLRS |
| | | EDTAVYYCARSIAVFWGDAFDIWGQGTMVT |
| | | VSS |
| SARS-CoV-2 | 399 | GYTFTSYY |
| S2X227-v1 | | |
| mAb CDRH1 | | |
| (aa) | | |
| SARS-CoV-2 | 400 | INPGGVST |
| S2X227-v1 | | |
| mAb CDRH2 | | |
| (aa) | | |
| SARS-CoV-2 | 401 | ARSIAVFWGDAFDI |
| S2X227-v1 | | |
| mAb CDRH3 | | |
| (aa) | | |
| SARS-CoV-2 | 402 | DIQMTQSPDSLAVSLGERATINCKSSQSVLYS |
| S2X227-v1 | | SNNKNYLAWYQQKPGQPPKLLIYWASTRES |
| mAb VL(VK) | | GVPDRFSGSGSGTDFTLTISSLQAEDVAVYYC |
| (aa) | | QQYSSSPLTFGGGTKVEIK |
| SARS-CoV-2 | 403 | QSVLYSSNNKNY |
| S2X227-v1 | | |
| mAb CDRL1 | | |
| (aa) | | |
| SARS-CoV-2 | 404 | WAS |
| S2X227-v1 | | |
| mAb CDRL2 | | |
| (aa) | | |
| SARS-CoV-2 | 405 | QQYSSSPLT |
| S2X227-v1 | | |
| mAb CDRL3 | | |
| (aa) | | |
| SARS-CoV-2 | 406 | GAGGTGCAGCTGGTGCAGTCTGGGGCTGAG |
| S2X227-v1 | | GTGAAGAAGCCTGGGGCCTCAGTGAAGGTT |
| mAb VH (nt) | | TCCTGCAAGGCATCTGGATACACCTTCAC |
| | | CAGCTACTATATGCACTGGGTGCGACAGG |
| | | CCCCTGGACAAGGGCTTGAGTGGATGGGAA |
| | | TAATCAACCCTGGTGGTGTTAGCACAACG |
| | | TACGCACATTACGCACAGAAGTTCCAGGGC |
| | | AGAGTCACCATGACCAGGGACACGTCCACG |
| | | AGCACAGTCTACATGGAGCTGAGCAGCCTG |
| | | AGATCTGAGGACACGGCCGTGTATTACTGT |
| | | GCGAGATCTATAGCAGTGTTTTGGGGAG |
| | | ATGCTTTTGATATCTGGGGCCAAGGGACA |
| | | ATGGTCACCGTCTCTTCAG |
| SARS-CoV-2 | 407 | GACATCCAGATGACCCAGTCTCCAGACTCC |
| S2X227-v1 | | CTGGCTGTGTCTCTGGGCGAGAGGGCCACC |
| mAb VL(VK) | | ATCAACTGCAAGTCCAGCCAGAGTGTTTT |
| (nt) | | ATACAGCTCCAACAATAAGAACTACTTAG |
| | | CTTGGTACCAGCAGAAACCAGGACAGCCTC |
| | | CTAAGCTGCTCATTTACTGGGCGTCTACCC |
| | | GGGAATCCGGGGTCCCTGACCGATTCAGTG |
| | | GCAGCGGGTCTGGGACAGATTTCACTCTCA |
| | | CCATCAGCAGCCTGCAGGCTGAAGATGTGG |
| | | CAGTTTATTACTGTCAGCAATATTCTAGTT |
| | | CTCCCCTCACTTTCGGCGGAGGGACCAAG |
| | | GTGGAGATCAAAC |
| SARS-CoV-2 | 408 | QVQLVQSGAEVKKPGSSVKVSCKASGGIFNT |
| S2X259-v1 | | YTISWVRQAPGQGLEWMGRIILMSGMANYA |
| mAb VH (aa) | | QKIQGRVTITADKSTSTAYMELTSLRSDDTAV |
| | | YYCARGENGNYYGWGDDDAFDISGQGTLV |
| | | TVYS |
| SARS-CoV-2 | 409 | GGIFNTYT |
| S2X259-v1 | | |
| mAb CDRH1 | | |
| (aa) | | |
| SARS-CoV-2 | 410 | IILMSGMA |
| S2X259-v1 | | |
| mAb CDRH2 | | |
| (aa) | | |
| SARS-CoV-2 | 411 | ARGENGNYYGWGDDDAFDI |
| S2X259-v1 | | |
| mAb CDRH3 | | |
| (aa) | | |
| SARS-CoV-2 | 412 | QTVLTQPPSVSGAPGQRVTISCTGSNSNIGAG |
| S2X259-v1 | | YDVHWYQQLPGTAPKLLICGNSNRPSGVPDR |
| mAb VL (aa) | | FSGSKSGTSASLAITGLQAEDEADYYCQSYDS |
| | | SLSGPNWVFGGGTKLTVL |
| SARS-CoV-2 | 413 | NSNIGAGYD |
| S2X259-v1 | | |
| mAb CDRL1 | | |
| (aa) | | |
| SARS-CoV-2 | 414 | GNS |
| S2X259-v1 | | |
| mAb CDRL2 | | |
| (aa) | | |
| SARS-CoV-2 | 415 | QSYDSSLSGPNWV |
| S2X259-v1 | | |
| mAb CDRL3 | | |
| (aa) | | |
| SARS-CoV-2 | 416 | CAGGTACAGCTGGTGCAATCTGGGGCTGAG |
| S2X259-v1 | | GTGAAGAAGCCTGGGTCCTCGGTGAAGGTC |
| mAb VH (nt) | | TCCTGCAAGGCTTCTGGGGGCATCTTCAA |
| | | CACCTATACTATCAGCTGGGTGCGACAGG |
| | | CCCCTGGGCAAGGGCTTGAGTGGATGGGAA |
| | | GGATCATCCTTATGTCTGGTATGGCAAAC |
| | | TACGCACAGAAGATCCAGGGCAGAGTCAC |
| | | GATAACCGCGGACAAATCGACGAGCACAG |
| | | CCTACATGGAGCTGACCAGCCTGAGATCTG |
| | | ACGACACGGCCGTCTATTACTGTGCGAGAG |
| | | GCTTCAACGGGAACTATTATGGTTGGGG |
| | | GGACGATGATGCTTTTGATATCTCGGGCC |
| | | AAGGGACACTGGTCACCGTCTATTCAG |
| SARS-CoV-2 | 417 | CAGACTGTGTTGACGCAGCCGCCCTCAGTG |
| S2X259-v1 | | TCTGGGGCCCCAGGGCAGAGGGTCACCATC |
| mAb VL (nt) | | TCCTGCACTGGGAGCAACTCCAACATCGG |
| | | GGCTGGTTATGATGTACACTGGTACCAGC |
| | | AGCTTCCAGGAACAGCCCCCAAACTCCTCA |
| | | TCTGTGGTAACAGCAATCGGCCCTCAGGG |
| | | GTCCCTGACCGATTCTCTGGCTCCAAGTCTG |
| | | GCACCTCAGCCTCCCTGGCCATCACTGGGC |
| | | TCCAGGCTGAGGATGAGGCTGATTATTACT |
| | | GCCAGTCCTATGACAGCAGCCTGAGTGG |
| | | CCCGAATTGGGTGTTCGGCGGAGGGACCA |
| | | AGCTGACCGTCCTAC |
| SARS-CoV-2 | 418 | EVQLVESGGGLIQPGGSLRLSCAASGFTVSSN |
| S2X288-v1 | | YMSWVRQAPGKGLEWVSVIYFGGTTYYAD |
| mAb VH (aa) | | SVKGRFTISRDTSENTLFLQMNSLRVEDTAVY |
| | | YCARDQGIAVAGLDFGAYDIWGQGTMVTV |
| | | SS |
| SARS-CoV-2 | 419 | GFTVSSNY |
| S2X288-v1 | | |
| mAb CDRH1 | | |
| (aa) | | |
| SARS-CoV-2 | 420 | IYFGGTT |
| S2X288-v1 | | |
| mAb CDRH2 | | |
| (aa) | | |
| SARS-CoV-2 | 421 | ARDQGIAVAGLDFGAYDI |
| S2X288-v1 | | |
| mAb CDRH3 | | |
| (aa) | | |
| SARS-CoV-2 | 422 | DIVMTQSPDSLAVSLGERATINCKSSQSVLYS |
| S2X288-v1 | | SNNKNYLAWYQQKPGQPPKLLIYWASTRES |
| mAb VL(VK) | | GVPDRFSGSGSGTDFTLTISSLQAEDVAVYYC |
| (aa) | | QQYYRTPTWTFGQGTKVEIK |
| SARS-CoV-2 | 423 | QSVLYSSNNKNY |
| S2X288-v1 | | |
| mAb CDRL1 | | |
| (aa) | | |
| SARS-CoV-2 | 424 | WAS |
| S2X288-v1 | | |
| mAb CDRL2 | | |
| (aa) | | |
| SARS-CoV-2 | 425 | QQYYRTPTWT |
| S2X288-v1 | | |
| mAb CDRL3 | | |
| (aa) | | |
| SARS-CoV-2 | 426 | GAGGTGCAGCTGGTGGAGTCTGGAGGAGG |
| S2X288-v1 | | CTTGATCCAGCCTGGGGGGTCCCTGAGACT |
| mAb VH (nt) | | CTCCTGTGCAGCCTCTGGGTTCACCGTCAG |
| | | TAGCAACTACATGAGCTGGGTCCGCCAGG |
| | | CTCCAGGGAAGGGGCTGGAGTGGGTCTCAG |
| | | TTATTTATTTCGGTGGTACCACATACTAC |
| | | GCAGACTCCGTGAAGGGCCGATTCACCATC |
| | | TCCAGAGACACTTCCGAGAACACGCTGTTT |
| | | CTTCAGATGAACAGCCTGAGAGTCGAGGAC |
| | | ACGGCCGTGTATTACTGTGCCAGAGATCA |
| | | GGGTATAGCAGTGGCTGGTCTCGATTTT |
| | | GGCGCTTATGATATCTGGGGCCAAGGGAC |
| | | AATGGTCACCGTCTCTTCAG |
| SARS-CoV-2 | 427 | GACATCGTGATGACCCAGTCTCCAGACTCC |
| S2X288-v1 | | CTGGCTGTGTCTCTGGGCGAGAGGGCCACC |
| mAb VL(VK) | | ATCAACTGCAAGTCCAGCCAGAGTGTTTT |
| (nt) | | ATATAGCTCCAACAATAAGAACTACTTAG |
| | | CTTGGTACCAGCAGAAGCCAGGACAGCCTC |
| | | CTAAGCTGCTCATTTACTGGGCATCTACCC |
| | | GGGAATCCGGGGTCCCTGACCGATTCAGTG |
| | | GCAGCGGGTCTGGGACAGATTTCACTCTCA |
| | | CCATCAGCAGCCTGCAGGCTGAAGATGTGG |
| | | CAGTTTATTACTGTCAGCAATATTATAGGA |
| | | CTCCCACGTGGACGTTCGGCCAAGGGACC |
| | | AAGGTGGAAATCAAAC |
| Antibody | 428 | QVQLVQSGAEVKKPGSSVKVSCKASGGIFNT |
| 407 10 1 v2 | | YTISWVRQAPGQGLEWMGRIILMSGMANYA |
| (VH) (aa) | | QKIQGRVTITADKSTSTAYMELTSLRSDDTAV |
| | | YYCARGFNGNYYGWGDDDAFDIWGQGTLV |
| | | TVYS |
| Antibody | 429 | CAGGTGCAGCTGGTCCAGAGCGGCGCAGA |
| 407_10_1_v2 | | GGTCAAAAAGCCCGGCAGTAGTGTCAAGGT |
| (VH) (nt) | | GTCTTGTAAAGCATCAGGGGGTATCTTCAA |
| | | CACCTACACAATCAGCTGGGTGAGACAGGC |
| | | TCCAGGACAGGGACTGGAGTGGATGGGCC |
| | | GCATCATCCTGATGTCTGGCATGGCCAATT |
| | | ACGCTCAGAAGATCCAGGGCAGGGTGACC |
| | | ATCACAGCCGACAAGTCCACCAGCACAGCT |
| | | TATATGGAGCTGACCTCTCTGAGGTCCGAC |
| | | GATACAGCCGTGTACTATTGCGCTCGGGGC |
| | | TTCAACGGCAATTACTATGGCTGGGGAGAT |
| | | GATGATGCTTTTGACATTTGGGGGCAGGGC |
| | | ACTCTGGTGACAGTCTACAGT |
| SARS-CoV-2 | 430 | GAAGTGCAACTAGTGCAAAGTGGTGCAGA |
| S2X227-v1 | | AGTCAAGAAGCCCGGCGCTTCTGTTAAAGT |
| (WT) (nt-CO) | | GTCCTGCAAGGCCTCTGGCTACACCTTTAC |
| | | ATCCTACTACATGCACTGGGTGCGGCAGGC |
| | | TCCTGGCCAGGGCCTGGAGTGGATGGGCAT |
| | | CATCAACCCTGGAGGAGTGAGCACCACCTA |
| | | CGCTCACTACGCCCAGAAGTTCCAGGGCAG |
| | | AGTGACAATGACCCGGGACACCTCCACCTC |
| | | TACCGTGTACATGGAACTGTCCTCTCTGAG |
| | | ATCTGAGGATACCGCTGTGTACTATTGTGC |
| | | CAGATCCATCGCCGTGTTCTGGGGCGACGC |
| | | CTTCGACATCTGGGGCCAAGGCACCATGGT |
| | | GACCGTGTCCAGC |
| SARS-CoV-2 | 431 | GATATCCAAATGACTCAAAGTCCAGATAGT |
| S2X227-v1 | | CTCGCTGTGTCCCTGGGCGAGCGGGCTACC |
| mAb VL(VK) | | ATCAACTGCAAGTCCAGCCAGTCCGTGCTG |
| (nt-CO) | | TACTCCTCCAATAACAAGAACTACCTGGCC |
| | | TGGTATCAACAGAAGCCTGGCCAGCCTCCA |
| | | AAGCTGCTGATCTACTGGGCCTCTACCAGA |
| | | GAGTCCGGCGTCCCCGATAGATTCTCCGGA |
| | | TCTGGCTCTGGCACCGACTTCACCCTGACC |
| | | ATCTCCTCTCTGCAGGCCGAGGACGTGGCC |
| | | GTGTACTACTGTCAGCAGTACAGCTCTTCTC |
| | | CTCTGACCTTTGGCGGCGGAACAAAAGTGG |
| | | AAATCAAG |
| Antibody | 432 | EVQLVQSGAEVKKPGASVKVSCKASGYTFTS |
| 407_10_2_v2 | | YYIHWVRQAPGQGLEWMGIINPGGVSTTYAH |
| VH (aa) | | YAQKFQGRVTMTRDTSTSTVYMELSSLRSED |
| | | TAVYYCARSIAVFWGDAFDIWGQGTMVTVS |
| | | S |
| Antibody | 433 | GAAGTGCAACTAGTGCAAAGTGGTGCAGA |
| 407_10_2_v2 | | AGTCAAGAAGCCCGGAGCTTCTGTGAAAGT |
| VH (nt-CO) | | GTCCTGCAAGGCCTCTGGCTACACCTTTACC |
| | | TCCTACTACATCCACTGGGTGCGGCAGGCT |
| | | CCTGGACAAGGCCTGGAGTGGATGGGCATC |
| | | ATCAACCCTGGCGGCGTGTCCACCACCTAC |
| | | GCTCACTACGCCCAGAAGTTCCAGGGCAGA |
| | | GTGACAATGACCAGAGATACCAGCACATCT |
| | | ACCGTGTACATGGAACTGTCCTCTCTGCGG |
| | | TCCGAGGACACCGCTGTGTACTATTGTGCC |
| | | AGATCCATCGCCGTGTTCTGGGGCGACGCC |
| | | TTCGACATCTGGGGCCAGGGCACCATGGTT |
| | | ACCGTGTCTAGC |
| Antibody | 434 | EVQLVQSGAEVKKPGASVKVSCKASGYTFTS |
| 407_10_2_v3 | | YYMHWVRQAPGQGLEWMGIINPGGVSTTYA |
| VH (aa) | | HYAQKFQGRVTMTRDTSTSTVYMELSSLRSE |
| | | DTAVYYCARSIAVFFGDAFDIWGQGTMVTVS |
| | | S |
| Antibody | 435 | ARSIAVFFGDAFDI |
| 407_10_2_v3 | | |
| CDRH3 (aa) | | |
| Antibody | 436 | GAAGTGCAACTAGTGCAAAGTGGTGCAGA |
| 407_10_2_v3 | | AGTCAAGAAGCCCGGAGCTTCTGTGAAAGT |
| VH | | GTCCTGCAAGGCCTCTGGCTACACCTTTACC |
| (nt-CO) | | TCCTACTACATGCACTGGGTCCGGCAGGCT |
| | | CCTGGACAAGGCCTGGAGTGGATGGGCATC |
| | | ATCAACCCTGGCGGCGTGTCTACCACCTAC |
| | | GCTCACTACGCCCAGAAGTTCCAGGGCAGA |
| | | GTGACAATGACCAGAGATACCAGCACCTCT |
| | | ACAGTGTACATGGAACTGTCCTCTCTGCGG |
| | | TCCGAGGACACCGCTGTGTACTATTGTGCC |
| | | AGATCCATCGCCGTGTTCTTCGGCGACGCC |
| | | TTCGACATCTGGGGCCAGGGCACCATGGTG |
| | | ACCGTGTCCAGC |
| Antibody | 437 | EVQLVQSGAEVKKPGASVKVSCKASGYTFTS |
| 407_10_2_v4 | | YYIHWVRQAPGQGLEWMGIINPGGVSTTYAH |
| VH (aa) | | YAQKFQGRVTMTRDTSTSTVYMELSSLRSED |
| | | TAVYYCARSIAVFFGDAFDIWGQGTMVTVSS |
| Antibody | 438 | GAAGTGCAACTAGTGCAAAGTGGTGCAGA |
| 407_10_2_v4 | | AGTGAAGAAGCCTGGCGCTTCTGTTAAAGT |
| VH (nt-CO) | | GTCCTGCAAGGCCTCTGGCTACACCTTTACC |
| | | TCCTACTACATCCACTGGGTGCGGCAGGCT |
| | | CCTGGACAAGGCCTGGAGTGGATGGGCATC |
| | | ATCAACCCCGGCGGCGTGTCTACCACCTAC |
| | | GCTCACTACGCCCAGAAGTTCCAGGGAAGA |
| | | GTGACCATGACCAGAGATACCAGCACATCT |
| | | ACAGTGTACATGGAACTGTCCTCTCTGCGG |
| | | TCCGAGGACACCGCTGTGTACTATTGTGCC |
| | | AGATCCATCGCCGTGTTCTTCGGCGACGCC |
| | | TTCGACATCTGGGGCCAGGGCACCATGGTC |
| | | ACCGTGTCCAGC |
| Antibody | 439 | DIQMTQSPDSLAVSLGERATINCKSSQSVLYS |
| 407_10_2_v5 | | SNNKNYLAWYQQKPGQPPKLLIYFASTRESG |
| VL (aa) | | VPDRFSGSGSGTDFTLTISSLQAEDVAVYYCQ |
| | | QYSSSPLTFGGGTKVEIK |
| Antibody | 440 | FAS |
| 407_10_2_v5 | | |
| CDRL2 (aa) | | |
| Antibody | 441 | GATATCCAAATGACTCAAAGTCCAGATAGT |
| 407_10_2_v5 | | CTCGCTGTGTCCCTGGGCGAGAGAGCTACA |
| VL | | ATCAACTGCAAGTCCAGCCAGTCCGTGCTG |
| (nt-CO) | | TACTCCTCCAATAACAAGAACTACCTGGCC |
| | | TGGTACCAGCAGAAACCAGGCCAGCCTCCT |
| | | AAGCTGCTGATCTACTTCGCCTCCACCAGA |
| | | GAGTCCGGCGTCCCCGATCGGTTTTCTGGCT |
| | | CTGGATCTGGCACCGACTTCACCCTGACCA |
| | | TCTCCTCTCTGCAGGCCGAGGACGTGGCCG |
| | | TGTACTACTGTCAGCAATATTCTAGCTCT |
| | | CCTCTGACCTTCGGCGGCGGAACCAAGGTG |
| | | GAAATCAAG |
| Antibody | 442 | QTVLTQPPSVSGAPGQRVTISCTGSNSNIGAG |
| S2X259-v3 | | YDVHWYQQLPGTAPKLLISGNSNRPSGVPDR |
| VL (aa) | | FSGSKSGTSASLAITGLQAEDEADYYCQSYDS |
| | | SLSGPNWVFGGGTKLTVL |
| Antibody | 443 | QTVLTQPPSVSGAPGQRVTISCTGSNSNIGAG |
| S2X259-v4 | | YDVHWYQQLPGTAPKLLIAGNSNRPSGVPDR |
| VL (aa) | | FSGSKSGTSASLAITGLQAEDEADYYCQSYDS |
| | | SLSGPNWVFGGGTKLTVL |
| Antibody | 444 | QTVLTQPPSVSGAPGQRVTISCTGSNSNIGAG |
| S2X259-v5 | | YDVHWYQQLPGTAPKLLIYGNSNRPSGVPDR |
| VL (aa) | | FSGSKSGTSASLAITGLQAEDEADYYCQSYDS |
| | | SLSGPNWVFGGGTKLTVL |
| Antibody | 445 | QTVLTQPPSVSGAPGQRVTISCTGSNSNIGAG |
| S2X259-v6 | | YDVHWYQQLPGTAPKLLIVGNSNRPSGVPDR |
| VL (aa) | | FSGSKSGTSASLAITGLQAEDEADYYCQSYDS |
| | | SLSGPNWVFGGGTKLTVL |
| Antibody | 446 | QVQLVQSGAEVKKPGSSVKVSCKASGGIFNT |
| S2X259-v7 | | YTISWVRQAPGQGLEWMGRIILISGIANYAQ |
| VH (aa) | | KIQGRVTITADKSTSTAYMELTSLRSDDTAVY |
| | | YCARGFNGNYYGWGDDDAFDISGQGTLVT |
| | | VYS |
| Antibody | 447 | IILISGIA |
| S2X259-v7 | | |
| CDRH2 (aa) | | |
| Antibody | 448 | QVQLVQSGAEVKKPGSSVKVSCKASGGIFST |
| S2X259-v8 | | YTISWVRQAPGQGLEWMGRIILMSGMANYA |
| VH (aa) | | QKIQGRVTITADKSTSTAYMELTSLRSDDTAV |
| | | YYCARGFNGNYYGWGDDDAFDISGQGTLV |
| | | TVYS |
| Antibody | 449 | GGIFSTYT |
| S2X259-v8 | | |
| CDRHI (aa) | | |
| SARS-CoV; | 450 | MFIFLLFLTL TSGSDLDRCT TFDDVQAPNY |
| Urbani strain; | | TQHTSSMRGV YYPDEIFRSD TLYLTQDLFL |
| surface | | PFYSNVTGFH TINHTFGNPV IPFKDGIYFA |
| glycoprotein; | | ATEKSNVVRG WVFGSTMNNK SQSVIIINNS |
| GenBank: | | TNVVIRACNF ELCDNPFFAV SKPMGTQTHT |
| AAP13441.1 | | MIFDNAFNCT FEYISDAFSL DVSEKSGNFK |
| | | HLREFVFKNK DGFLYVYKGY QPIDVVRDLP |
| | | SGFNTLKPIF KLPLGINITN FRAILTAFSP |
| | | AQDIWGTSAA AYFVGYLKPT TFMLKYDENG |
| | | TITDAVDCSQ NPLAELKCSV KSFEIDKGIY |
| | | QTSNFRVVPS GDVVRFPNIT NLCPFGEVEN |
| | | ATKFPSVYAW ERKKISNCVA DYSVLYNSTF |
| | | FSTFKCYGVS ATKLNDLCFS NVYADSFVVK |
| | | GDDVRQIAPG QTGVIADYNY KLPDDFMGCV |
| | | LAWNTRNIDA TSTGNYNYKY RYLRHGKLRP |
| | | FERDISNVPF SPDGKPCTPP ALNCYWPLND |
| | | YGFYTTTGIG YQPYRVVVLS FELLNAPATV |
| | | CGPKLSTDLI KNQCVNFNFN GLTGTGVLTP |
| | | SSKRFQPFQQ FGRDVSDFTD SVRDPKTSEI |
| | | LDISPCSFGG VSVITPGTNA SSEVAVLYQD |
| | | VNCTDVSTAI HADQLTPAWR IYSTGNNVFQ |
| | | TQAGCLIGAE HVDTSYECDI PIGAGICASY |
| | | HTVSLLRSTS QKSIVAYTMS LGADSSIAYS |
| | | NNTIAIPTNF SISITTEVMP VSMAKTSVDC |
| | | NMYICGDSTE CANLLLQYGS FCTQLNRALS |
| | | GIAAEQDRNT REVFAQVKQM YKTPTLKYFG |
| | | GFNFSQILPD PLKPTKRSFI EDLLFNKVTL |
| | | ADAGFMKQYG ECLGDINARD LICAQKENGL |
| | | TVLPPLLTDD MIAAYTAALV SGTATAGWTF |
| | | GAGAALQIPF AMQMAYRENG IGVTQNVLYE |
| | | NQKQIANQFN KAISQIQESL TTTSTALGKL |
| | | QDVVNQNAQA LNTLVKQLSS NFGAISSVLN |
| | | DILSRLDKVE AEVQIDRLIT GRLQSLQTYV |
| | | TQQLIRAAEI RASANLAATK MSECVLGQSK |
| | | RVDFCGKGYH LMSFPQAAPH |
| | | GVVFLHVTYV PSQERNFTTA PAICHEGKAY |
| | | FPREGVFVFN GTSWFITQRN FFSPQIITTD |
| | | NTFVSGNCDV VIGIINNTVY DPLQPELDSF |
| | | KEELDKYFKN HTSPDVDLGD ISGINASVVN |
| | | IQKEIDRLNE VAKNLNESLI DLQELGKYEQ |
| | | YIKWPWYVWL GFIAGLIAIV MVTILLCCMT |
| | | SCCSCLKGAC SCGSCCKFDE DDSEPVLKGV |
| | | KLHYT |
| CMV | 451 | GACATTGATTATTGACTAGTTATTAATAGTA |
| promoter (nt) | | ATCAATTACGGGGTCATTAGTTCATAGCCC |
| | | ATATATGGAGTTCCGCGTTACATAACTTAC |
| | | GGTAAATGGCCCGCCTGGCTGACCGCCCAA |
| | | CGACCCCCGCCCATGACGTCAATAATGACG |
| | | TATGTTCCCATAGTAACGCCAATAGGGACT |
| | | TTCCATTGACGTCAATGGGTGGAGTATTTA |
| | | CGGTAAACTGCCCACTTGGCAGTACATCAA |
| | | GTGTATCATATGCCAAGTACGCCCCCTATT |
| | | GACGTCAATGACGGTAAATGGCCCGCCTGG |
| | | CATTATGCCCAGTACATGACCTTATGGGAC |
| | | TTTCCTACTTGGCAGTACATCTACGTATTAG |
| | | TCATCGCTATTACCATGGTGATGCGGTTTTG |
| | | GCAGTACATCAATGGGCGTGGATAGCGGTT |
| | | TGACTCACGGGGATTTCCAAGTCTCCACCC |
| | | CATTGACGTCAATGGGAGTTTGTTTTGGCA |
| | | CCAAAATCAACGGGACTTTCCAAAATGTCG |
| | | TAACAACTCCGCCCCATTGACGCAAATGGG |
| | | CGGTAGGCGTGTACGGTGGGAGGTCTATAT |
| | | AAGCAGAGCTCGTTTAGTGAACCGTCAGAT |
| | | CGCCTGGAGACGCCATCCACGCTGTTTTGA |
| | | CCTCCATAGAAGACACCGGGACCGATCCAG |
| | | CCTCCGCGGCCGGGAACGGTGCATTGGAAC |
| | | GCGGATTCCCCGTGCCAAGAGTGACGTAAG |
| | | TACCGCCTATAGAGTCTATAGGCCCACCCC |
| | | CTTGGCTTCGTTAG |
| Signal peptide | 452 | ATGGGATGGTCATGTATCATCCTTTTTCTAG |
| (nt) | | TAGCAACTGCAACCGGTGT |
| Poly- | 453 | AACTTGTTTATTGCAGCTTATAATGGTTACA |
| adenylation | | AATAAAGCAATAGCATCACAAATTTCACAA |
| signal | | ATAAAGCATTTTTTTCACTGCATTCTAGTTG |
| sequence (nt) | | TGGTTTGTCCAAACTCATCAATGTATCTTAT |
| | | CATGTCTGGATC |
| Signal peptide | 454 | MGWSCIILFLVATATG |
| (aa) | | |
| Signal peptide | 455 | atgggctggtcctgcatcatcctgttcctggtggccacagccaccggcg |
| (nt-CO) | | tgcacagc |
| Signal peptide | 456 | MGWSCIILFLVATATGVHS |
| (aa) | | |
| S2X259 | 457 | IILMSGKA |
| variant | | |
| CDRH2 | | |
| S2X259 | 458 | QVQLVQSGAEVKKPGSSVKVSCKASGGIFNT |
| variant VH | | YTISWVRQAPGQGLEWMGRIILMSGMANYA |
| W118_S127 | | QKIQGRVTITADKSTSTAYMELTSLRSDDTAV |
| | | YYCARGENGNYYGWGDDDAFDIWGQGTLV |
| | | TVSS |
| S2X259 | 459 | QVQLVQSGAEVKKPGSSVKVSCKASGGIFNT |
| variant VH | | YTISWVRQAPGQGLEWMGRIILMSGKANYA |
| M64K W118 | | QKIQGRVTITADKSTSTAYMELTSLRSDDTAV |
| S127 | | YYCARGFNGNYYGWGDDDAFDIWGQGTLV |
| | | TVSS |
| S2X259 | 460 | QVQLVQSGAEVKKPGSSVKVSCKASGGIFNT |
| variant VH | | YTISWVRQAPGQGLEWMGRIILISGIANYAQ |
| M59I M64I | | KIQGRVTITADKSTSTAYMELTSLRSDDTAVY |
| W118 S127 | | YCARGFNGNYYGWGDDDAFDIWGQGTLVT |
| | | VSS |
| ACE2 | 461 | CAAGAGCAAACGGTTGAACAC |
| forward | | |
| primer | | |
| ACE2 reverse | 462 | CCAGAGCCTCTCATTGTAGTCT |
| primer | | |
| HPRT | 463 | CCTGGCGTCGTGATTAGTG |
| forward | | |
| primer | | |
| HPRT reverse | 464 | ACACCCTTTCCAAATCCTCAG |
| primer | | |
| TMPRSS2 | 465 | CAAGTGCTCCRACTCTGGGAT |
| forward | | |
| primer | | |
| TMPRSS2 | 466 | AACACACCGRTTCTCGTCCTC |
| reverse primer | | |

The present disclosure also provides the following Embodiments:

Embodiment 1. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein: (i) the CDRH1 comprises or consists of the amino acid sequence according to any one of SEQ ID NOs.: 409, 23, 33, 38, 46, 53, 55, 63, 70, 72, 83, 93, 103, 113, 123, 137, 147, 160, 166, 181, 191, 201, 211, 221, 233, 243, 268, 305, 315, 325, 330, 335, 349, 359, 369, 379, 389, 399, 419, or 449, or a sequence variant thereof comprising one, two, or three amino acid substitutions, one or more of which substitutions is optionally a conservative substitution and/or is a substitution to a germline-encoded amino acid; (ii) the CDRH2 comprises or consists of the amino acid sequence according to any one of SEQ ID NOs.: 410, 24, 31, 36, 39, 48, 51, 56, 65, 67, 73, 83, 93, 103, 113, 123, 137, 147, 161, 167, 182, 192, 202, 212, 222, 234, 244, 263, 269, 285, 287, 289, 293, 299, 301, 306, 316, 326, 331, 336, 350, 360, 370, 380, 390, 400, 420, 447, 457, or a sequence variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence according to any one of SEQ ID NOs.: 411, 25, 40, 57, 74, 84, 94, 104, 114, 124, 138, 148, 156, 162, 168, 183, 193, 203, 213, 223, 235, 245, 254, 257, 259, 261, 265, 271, 273, 275, 277, 279, 281, 290, 294, 296, 307, 317, 324, 327, 332, 337, 351, 361, 371, 381, 391, 401, 421, or 435, or a sequence variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence according to any one of SEQ ID NOs.: 413, 27, 42, 59, 76, 86, 96, 106, 116, 126, 140, 150, 163, 169, 185, 195, 205, 215, 225, 237, 247, 309, 319, 328, 333, 338, 353, 363, 373, 383, 393, 403, or 423, or a sequence variant thereof comprising one, two, or three amino acid substitutions, one or more of which substitutions is optionally a conservative substitution and/or is a substitution to a germline-encoded amino acid; (v) the CDRL2 comprises or consists of the amino acid sequence according to any one of SEQ ID NOs.: 414, 28, 43, 60, 77, 87, 97, 107, 117, 127, 141, 151, 164, 170, 186, 196, 206, 216, 226, 238, 248, 310, 320, 329, 334, 339, 354, 364, 374, 384, 394, 404, 424, or 440, or a sequence variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence according to any one of SEQ ID NOs.: 415, 29, 44, 61, 78, 88, 98, 108, 118, 128, 142, 152, 165, 171, 187, 197, 207, 217, 227, 239, 249, 283, 303, 311, 321, 355, 365, 375, 385, 395, 405, or 425, or a sequence variant thereof comprising having one, two, or three amino acid substitutions, one or more of which substitutions is optionally a conservative substitution and/or is a substitution to a germline-encoded amino acid, wherein the antibody or antigen binding fragment is capable of binding to a SARS-CoV-2 surface glycoprotein (S) expressed on a cell surface of a host cell, on a SARS-CoV-2 virion, or both.

Embodiment 2. The antibody or antigen-binding fragment of Embodiment 1, which is capable of neutralizing a SARS-CoV-2 infection in an in vitro model of infection and/or in an in vivo animal model of infection and/or in a human, wherein, optionally, the SARS-CoV-2 infection comprises a SARS-CoV-2 comprising the amino acid sequence according to SEQ ID NO.:3.

Embodiment 3. The antibody or antigen-binding fragment of Embodiment 1 or 2, which is (i) capable of binding to the surface glycoprotein of two or more (e.g., two, three, four, five, or more) sarbecoviruses expressed on a cell surface of a host cell, on a sarbecovirus virion, or both; and/or (ii) capable of neutralizing an infection by two or more sarbecoviruses in an in vitro model of infection and/or in an in vivo animal model of infection and/or in a human.

Embodiment 4. The antibody or antigen-binding fragment of any one of Embodiments 1-3, comprising CDRH1, CDRH2, CDRH3, CDRL1, CDRL2, and CDRL3 amino acid sequences according to SEQ ID NOs.: (i) 409-411 and 413-415, respectively; (ii) 23 or 160, 31, 25 or 162, and 27-29 or 163-165, respectively; (iii) 33, 24 or 161, 25 or 162, and 27-29 or 163-165, respectively; (iv) 33, 31, 25 or 162, and 27-29 or 163-165, respectively; (v) 33, 36, 25 or 162, and 27-29 or 163-165, respectively; (vi) 38-40 and 42-44, respectively; (vii) 46, 39 or 167, 40 or 168, and 42-44 or 169-171, respectively; (viii) 38 or 166, 48, 40 or 168 and 42-44 or 169-171, respectively; (ix) 46, 48, 40 or 168 and 42-44 or 169-171, respectively; (x) 46, 51, 40 or 168, and 42-44 or 169-171, respectively; (xi) 53, 48, 40 or 168, and 42-44 or 169-171, respectively; (xii) 55-57 and 59-61, respectively; (xiii) 63, 56, 57 and 59-61, respectively; (xiv) 55, 65, 57 and 59-61, respectively; (xv) 63, 67, 57, and 59-61, respectively; (xvi) 63, 65, 57 and 59-61, respectively; (xvii) 70, 65, 57, and 59-61, respectively; (xviii) 72-74 and 76-78, respectively; (xix) 82-84 and 86-88, respectively; (xx) 92-94 and 96-98, respectively; (xxi) 102-104, and 106-108, respectively; (xxii) 112-114 and 116-118, respectively; (xxiii); 122-124 and 126-128, respectively; (xxiv) 136-138 and 140-142, respectively; (xxv); 146-148 and 150-152, respectively; (xxvi) 112, 113, 156 and 116-118, respectively; (xxvii) 181-183 and 185-187, respectively; (xxviii) 191-193 and 195-197, respectively; (xxix) 201-203 and 205-207, respectively; (xxx) 211-213 and 215-217, respectively; (xxxi) 221-223 and 225-227, respectively; (xxxii) 233-235 and 237-239, respectively; (xxxiii) 243-245 and 247-249 respectively; (xxxiv) 211, 212, any one of 254, 257, 259, 261, or 324 and 215-217, respectively; (xxxv) any one of 221, 268, or 325, any one of 222, 263, 269, or 326, any one of 223, 265, 271, 273, or 327 and 225, 226 or 328, and 227 or 329, respectively; (xxxvi) 233 or 330, 234 or 331, any one of 235, 275, 277, 279, 281, or 332, and any one of 237, 282 or 333, 238 or 234, and 239, respectively; (xxxvii) 243 or 335, any one of 244, 285, 287, 289, 293, 299, 301, or 336, any one of 245, 290, 294, 296, or 337, and 247 or 338, 248 or 339, and 249 or 303, respectively; (xxxviii) 305-307 and 309-311, respectively; (xxxix) 315-317 and 319-321, respectively; (xxxx) 349-351 and 353-355, respectively; (xxxxi) 359-361 and 363-365, respectively; (xxxxii) 369-371 and 373-375, respectively; (xxxxiii) 379-381 and 383-385, respectively; (xxxxiv) 389-391 and 393-395, respectively; (xxxxv) 399, 400, 401 or 435, and 403, 404 or 440, and 405, respectively; (xxxxvi) 23-25 and 27-29, respectively; (xxxxvii) 419-421 and 423-425, respectively; (xxxxviii) 409, 447, 411, and 413-415, respectively; (xxxxix) 449, 410, 411, and 413-415, respectively; (xxxxx) 449, 447, 411, and 413-415, respectively; (xxxxxi) 409, 457, 411, and 413-415, respectively; (xxxxxii) 449, 457, 411, and 413-415, respectively.

Embodiment 5. An antibody, or an antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, the CDRH2, the CDRH3, the CDRL1, the CDRL2, and the CDRL3 comprise or consist of the amino acid sequences set forth in: (i) SEQ ID NOs.: 409, 410, 411, 413, 414, and 415, respectively; (ii) SEQ ID NOs.: 409, 447, 411, 413, 414, and 415, respectively; (iii) SEQ ID NOs.: 409, 457, 411, 413, 414, and 415, respectively; (iv) SEQ ID NOs.: 449, 410, 411, 413, 414, and 415, respectively; (v) SEQ ID NOs.: 449, 447, 411, 413, 414, and 415, respectively; or (vi) SEQ ID NOs.: 449, 457, 411, 413, 414, and 415, respectively, and wherein the antibody or antigen-binding fragment is capable of binding to a SARS-CoV-2 surface glycoprotein (S).

Embodiment 6. The antibody or antigen-binding fragment of any one of Embodiments 1-5, wherein the CDRH1, the CDRH2, the CDRH3, the CDRL1, the CDRL2, and the CDRL3 comprise or consist of the amino acid sequences set forth in SEQ ID NOs.: 409, 410, 411, 413, 414, and 415, respectively.

Embodiment 7. The antibody or antigen-binding fragment of any one of Embodiments 1-5, wherein the CDRH1, the CDRH2, the CDRH3, the CDRL1, the CDRL2, and the CDRL3 comprise or consist of the amino acid sequences set forth in SEQ ID NOs.: 409, 447, 411, 413, 414, and 415, respectively.

Embodiment 8. The antibody or antigen-binding fragment of any one of Embodiments 1-5, wherein the CDRH1, the CDRH2, the CDRH3, the CDRL1, the CDRL2, and the CDRL3 comprise or consist of the amino acid sequences set forth in SEQ ID NOs.: 409, 457, 411, 413, 414, and 415, respectively.

Embodiment 9. The antibody or antigen-binding fragment of any one of Embodiments 1-5, wherein the CDRH1, the CDRH2, the CDRH3, the CDRL1, the CDRL2, and the CDRL3 comprise or consist of the amino acid sequences set forth in SEQ ID NOs.: 449, 410, 411, 413, 414, and 415, respectively.

Embodiment 10. The antibody or antigen-binding fragment of any one of Embodiments 1-5, wherein the CDRH1, the CDRH2, the CDRH3, the CDRL1, the CDRL2, and the CDRL3 comprise or consist of the amino acid sequences set forth in SEQ ID NOs.: 449, 447, 411, 413, 414, and 415, respectively.

Embodiment 11. The antibody or antigen-binding fragment of any one of Embodiments 1-5, wherein the CDRH1, the CDRH2, the CDRH3, the CDRL1, the CDRL2, and the CDRL3 comprise or consist of the amino acid sequences set forth in SEQ ID NOs.: 449, 457, 411, 413, 414, and 415, respectively.

Embodiment 12. An antibody, or an antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, the CDRH2, the CDRH3, the CDRL1, the CDRL2, and the CDRL3 comprise or consist of the amino acid sequences set forth in:
(i) SEQ ID NOs.: 399, 400, 401, 403, 404, and 405, respectively;
(ii) SEQ ID NOs.: 399, 400, 435, 403, 404, and 405, respectively;
(iii) SEQ ID NOs.: 399, 400, 401, 403, 440, and 405, respectively; or
(iv) SEQ ID NOs.: 399, 400, 435, 403, 440, and 405, respectively,
and wherein the antibody or antigen-binding fragment is capable of binding to a SARS-CoV-2 surface glycoprotein (S).

Embodiment 13. The antibody or antigen-binding fragment of any one of Embodiments 1-4 and 12, wherein the CDRH1, the CDRH2, the CDRH3, the CDRL1, the CDRL2, and the CDRL3 comprise or consist of the amino acid sequences set forth in SEQ ID NOs.: 399, 400, 401, 403, 404, and 405, respectively.

Embodiment 14. The antibody or antigen-binding fragment of any one of Embodiments 1-4 and 12, wherein the CDRH1, the CDRH2, the CDRH3, the CDRL1, the CDRL2, and the CDRL3 comprise or consist of the amino acid sequences set forth in SEQ ID NOs.: 399, 400, 435, 403, 404, and 405, respectively.

Embodiment 15. The antibody or antigen-binding fragment of any one of Embodiments 1-4 and 12, wherein the CDRH1, the CDRH2, the CDRH3, the CDRL1, the CDRL2, and the CDRL3 comprise or consist of the amino acid sequences set forth in SEQ ID NOs.: 399, 400, 401, 403, 440, and 405, respectively.

Embodiment 16. The antibody or antigen-binding fragment of any one of Embodiments 1-4 and 12, wherein the CDRH1, the CDRH2, the CDRH3, the CDRL1, the CDRL2, and the CDRL3 comprise or consist of the amino acid sequences set forth in SEQ ID NOs.: 399, 400, 435, 403, 440, and 405, respectively.

Embodiment 17. The antibody or antigen-binding fragment of any one of Embodiments 1-16, wherein: (i) the VH comprises or consists of an amino acid sequence having at least 85% identity to the amino acid sequence according to any one of SEQ ID NOs.: 22, 30, 32, 34, 35, 37, 45, 47, 49, 50, 52, 54, 62, 64, 66, 68, 69, 71, 81, 91, 101, 111, 121, 135, 145, 155, 158, 180, 190, 200, 210, 220, 232, 242, 252, 253, 255, 256, 258, 260, 262, 264, 266, 267, 270, 272, 274, 276, 278, 280, 284, 286, 288, 291, 292, 295, 297, 298, 300, 304, 314, 348, 358, 368, 378, 388, 398, 408, 418, 428, 432, 434, 437, 446, 448, 448, 459, and 460, wherein the variation is optionally limited to one or more framework regions and/or the variation comprises one or more substitution to a germline-encoded amino acid; and/or (ii) the VL comprises or consists of an amino acid sequence having at least 85% identity to the amino acid sequence according to any one of SEQ ID NOs.: 26, 41, 58, 75, 85, 95, 105, 115, 125, 139, 149, 184, 194, 204, 214, 224, 230, 236, 246, 282, 302, 308, 319, 352, 362, 372, 382, 392, 402, 412, 422, 439, 442, 443, 444, and 445, wherein the variation is optionally limited to one or more framework regions and/or the variation comprises one or more substitution to a germline-encoded amino acid.

Embodiment 18. The antibody or antigen-binding fragment of any one of Embodiments 1-17, wherein the VH comprises or consists of any VH amino acid sequence set forth in Table 3, and wherein the VL comprises or consists of any VL amino acid sequence set forth in Table 3, wherein, optionally, the VH and the VL comprise or consist of the amino acid sequences according to SEQ ID NOs.:
(i) any one of 408, 446, 448, 458, 459, and 460 and any one of 412, 442, 443, 444, and 445, respectively, optionally according to (a) 408 and 412, respectively; (b) 408 and 442, respectively; (c) 408 and 443, respectively; (d) 408 and 444, respectively; (e) 408 and 445, respectively; (f) 428 and 412, respectively; (g) 428 and 442, respectively; (h) 428 and 443, respectively; (i) 428 and 444, respectively; (j) 428 and 445, respectively; (k) 446 and 412, respectively; (l) 446 and 442, respectively; (m) 446 and 443, respectively; (n) 446 and 444, respectively; (o) 446 and 445, respectively; (p) 448 and 412, respectively; (q) 448 and 442, respectively; (r) 448 and 443, respectively; (s) 448 and 444, respectively; (t) 448 and 445, respectively; (u) 458 and 412, respectively; (v) 458 and 442, respectively; (w) 458 and 443, respectively; (x) 458 and 444, respectively; (y) 458 and 445, respectively; (z) 459 and 412, respectively; (aa) 459 and 442, respectively; (bb) 459 and 443, respectively; (cc) 459 and 444, respectively; (dd) 459 and 445, respectively; (ee) 460 and 412, respectively; (ff) 460 and 442, respectively; (gg) 460 and 443, respectively; (hh) 460 and 444, respectively; or (ii) 460 and 445, respectively; (ii) 30 and 26, respectively; (iii) 32 and 26, respectively; (iv) 34 and 26, respectively; (v) 35 and 26, respectively; (vi) 37 and 41, respectively; (vii) 45 and 41, respectively; (viii) 47 and 41, respectively;
(ix) 49 and 41, respectively; (x) 50 and 41, respectively; (xi) 52 and 41, respectively; (xii) 54 and 58, respectively; (xiii) 62 and 58, respectively; (xiv) 64 and 58, respectively; (xv) 66 and 58, respectively; (xvi) 68 and 58, respectively; (xvii) 69 and 58, respectively; (xviii) 71 and 75, respectively; (xix) 81 and 85, respectively; (xx) 91 and 95, respectively; (xxi) 101 or 158 and 105, respectively; (xxii) 111 or 155 and 115, respectively; (xxiii) 121 and 125, respectively; (xxiv) 135 and 139, respectively; (xxv) 145 and 149, respectively; (xxvi) 180 and 184, respectively; (xxvii) 190 and 194, respectively; (xxviii) 200 and 204, respectively; (xxix) 210 and 214, respectively; (xxx) 220 and 224, respectively;
(xxxi) 220 and 230, respectively; (xxxii) 232 and 236, respectively; (xxxiii) 242 and 246, respectively; (xxxiv) any one of 252, 253, 255, 256, 258, or 260 and 214, respectively; (xxxv) any one of 262, 264, 266, 267, 270, or 272 and 224, respectively; (xxxvi) any one of 274, 276, 278, or 280 and 236 or 282, respectively (xxxvii) any one of 284, 286, 288, 291, 292, 295, 297, or 300 and 246 or 302, respectively; (xxxviii) 304 and 308, respectively; (xxxix) 314 and 318, respectively; (xxxx) 348 and 352, respectively; (xxxxi) 358 and 362, respectively; (xxxxii) 368 and 372, respectively; (xxxxiii) 378 and 382, respectively; (xxxxiv) 388 and 392, respectively; (xxxxv) 398 or 432 or 434 or 437 and 402 or 439, respectively, optionally (a) 398 and 402, respectively, (b) 398 and 439, respectively, (c) 432 and 402, respectively, (d) 432 and 439, respectively, (e) 434 and 402, respectively, (f) 434 and 439, respectively, (g) 437 and 402, respectively, or (h) 437 and 439, respectively; (xxxxvi) 408 or 428 and 412, respectively; (xxxxvii) 418 and 422, respectively; (xxxxviii) any one of 408, 446, 448, 458, 459, and 460 and any one of 412, 442, 443, 444, and 445, respectively; or (xxxxix) 22 and 26, respectively.

Embodiment 19. The antibody or antigen-binding fragment thereof of any one of Embodiments 1-18, wherein the VH and the VL have at least 85% identity to, or comprises or consist of, the amino acid sequences set forth in SEQ ID NOs.: (a) 458 and 445, respectively; (b) 408 and 442, respectively; (c) 408 and 443, respectively; (d) 408 and 444, respectively; (e) 408 and 445, respectively; (f) 428 and 412, respectively; (g) 428 and 442, respectively; (h) 428 and 443, respectively; (i) 428 and 444, respectively; (j) 428 and 445, respectively; (k) 446 and 412, respectively; (l) 446 and 442, respectively; (m) 446 and 443, respectively; (n) 446 and 444, respectively; (o) 446 and 445, respectively; (p) 448 and 412, respectively; (q) 448 and 442, respectively; (r) 448 and 443, respectively; (s) 448 and 444, respectively; (t) 448 and 445, respectively; (u) 458 and 412, respectively; (v) 458 and 442, respectively; (w) 458 and 443, respectively; (x) 458 and 444, respectively; (y) 408 and 412, respectively; (z) 459 and 412, respectively; (aa) 459 and 442, respectively; (bb) 459 and 443, respectively; (cc) 459 and 444, respectively; (dd) 459 and 445, respectively; (ee) 460 and 412, respectively; (ff) 460 and 442, respectively; (gg) 460 and 443, respectively; (hh) 460 and 444, respectively; or (ii) 460 and 445, respectively.

Embodiment 20. An antibody, or an antigen-binding fragment thereof, comprising a heavy chain variable domain (VH) comprising or consisting of the amino acid sequence set forth in SEQ ID NO.:458 and a light chain variable domain (VL) comprising or consisting of the amino acid sequence set forth in SEQ ID NO.:445,
wherein the antibody or antigen-binding fragment is capable of binding to a SARS-CoV-2 surface glycoprotein (S).

Embodiment 21. An antibody, or an antigen-binding fragment thereof, comprising a heavy chain variable domain (VH) comprising or consisting of the amino acid sequence set forth in SEQ ID NO.:408 and a light chain variable domain (VL) comprising or consisting of the amino acid sequence set forth in SEQ ID NO.:412,
wherein the antibody or antigen-binding fragment is capable of binding to a SARS-CoV-2 surface glycoprotein (S).

Embodiment 22. An antibody, or an antigen-binding fragment thereof, comprising a heavy chain variable domain (VH) comprising or consisting of the amino acid sequence set forth in SEQ ID NO.:460 and a light chain variable domain (VL) comprising or consisting of the amino acid sequence set forth in SEQ ID NO.:445, wherein the antibody or antigen-binding fragment is capable of binding to a SARS-CoV-2 surface glycoprotein (S).

Embodiment 23. An antibody, or an antigen-binding fragment thereof, comprising a heavy chain variable domain (VH) comprising or consisting of the amino acid sequence set forth in SEQ ID NO.:459 and a light chain variable domain (VL) comprising or consisting of the amino acid sequence set forth in SEQ ID NO.:445, wherein the antibody or antigen-binding fragment is capable of binding to a SARS-CoV-2 surface glycoprotein (S).

Embodiment 24. The antibody or antigen-binding fragment of any one of Embodiments 1-18, wherein the VH and the VL have at least 85% identity to the amino acid sequences set forth in SEQ ID NOs.: (i) 398 and 402, respectively; (ii) 398 and 439, respectively; (iii) 432 and 402, respectively; (iv) 432 and 439, respectively; (v) 434 and 402, respectively; (vi) 434 and 439, respectively; (vii) 437 and 402, respectively; or (viii) 437 and 439, respectively.

Embodiment 25. An antibody, or an antigen-binding fragment thereof, comprising a heavy chain variable domain (VH) and a light chain variable domain (VL), wherein the VH and the VL comprise or consist of the amino acid sequences set forth in SEQ ID NOs.: (i) 398 and 402, respectively; (ii) 398 and 439, respectively;
(iii) 432 and 402, respectively; (iv) 432 and 439, respectively; (v) 434 and 402, respectively; (vi) 434 and 439, respectively; (vii) 437 and 402, respectively; or (viii) 437 and 439, respectively, and wherein the antibody or antigen-binding fragment is capable of binding to a SARS-CoV-2 surface glycoprotein (S).

Embodiment 26. An antibody, or antigen-binding fragment thereof, comprising a heavy chain variable domain (VH) and a light chain variable domain (VL), wherein the VH comprises or consists of the amino acid sequence as set forth in SEQ ID NO: 22 and the VL comprises or consists of the amino acid sequence as set forth in SEQ ID NO: 26, and wherein the antibody or antigen-binding fragment is capable of binding to a SARS-CoV-2 surface glycoprotein (S).

Embodiment 27. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 23-25, respectively, and the CDRL1, CDRL2, and CDRL3 comprise or consist of the amino acid sequences set forth in SEQ ID NOs: 27-29, respectively, and wherein the antibody or antigen-binding fragment is capable of binding to a SARS-CoV-2 surface glycoprotein (S).

Embodiment 28. An antibody, or antigen-binding fragment thereof, comprising a heavy chain variable domain (VH) and a light chain variable domain (VL), wherein the VH comprises or consists of the amino acid sequence as set forth in SEQ ID NO: 37 and the VL comprises or consists of the amino acid sequence as set forth in SEQ ID NO: 41, and wherein the antibody or antigen-binding fragment is capable of binding to a SARS-CoV-2 surface glycoprotein (S).

Embodiment 29. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 38-40, respectively, and the CDRL1, CDRL2, and CDRL3 comprise or consist of the amino acid sequences set forth in SEQ ID NOs: 42-44, respectively, and wherein the antibody or antigen-binding fragment is capable of binding to a SARS-CoV-2 surface glycoprotein (S).

Embodiment 30. An antibody, or antigen-binding fragment thereof, comprising a heavy chain variable domain (VH) and a light chain variable domain (VL), wherein the VH comprises or consists of the amino acid sequence as set forth in SEQ ID NO: 54 and the VL comprises or consists of the amino acid sequence as set forth in SEQ ID NO: 58, and wherein the antibody or antigen-binding fragment is capable of binding to a SARS-CoV-2 surface glycoprotein (S).

Embodiment 31. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 55-57, respectively, and the CDRL1, CDRL2, and CDRL3 comprise or consist of the amino acid sequences set forth in SEQ ID NOs: 59-61, respectively, and wherein the antibody or antigen-binding fragment is capable of binding to a SARS-CoV-2 surface glycoprotein (S).

Embodiment 32. The antibody or antigen-binding fragment of any one of Embodiments 1-31, which is capable of specifically binding to a SARS-CoV-2 surface glycoprotein (S).

Embodiment 33. The antibody or antigen-binding fragment of any one of Embodiments 1-32, which: (i) recognizes an epitope in the ACE2 receptor binding motif (RBM, SEQ ID NO.:5) of SARS-CoV-2; (ii) is capable of blocking an interaction between SARS-CoV-2 RBD (e.g., SARS-CoV-2 RBM) and ACE2; (iii) is capable of binding to SARS-CoV-2 S protein with greater avidity that to SARS-CoV S protein; (iv) recognizes an epitope that is conserved in the ACE2 RBD of SARS-CoV-2 and in an ACE2 RBD of SARS-CoV; (v) is cross-reactive against SARS-CoV-2 and SARS-CoV coronavirus; (vii) recognizes an epitope in the SARS-CoV-2 surface glycoprotein that is not in the ACE2 RBM; or (viii) any combination of (i)-(vii).

Embodiment 34. The antibody or antigen-binding fragment of any one of Embodiments 1-33, which: (i) recognizes an epitope in a S protein of two or more sarbecoviruses; (ii) is capable of blocking an interaction between a S protein of one, two, or more sarbecoviruses and a cell surface receptor; (iii) recognizes an epitope that is conserved in the Spike protein of two or more sarbecoviruses; (iv) is cross-reactive against two or more sarbecoviruses; or (v) any combination of (i)-(iv).

Embodiment 35. The antibody or antigen-binding fragment of any one of Embodiments 1-34, which is capable of capable of inhibiting an interaction between SARS-CoV-2 and any one or more of DC-SIGN, L-SIGN, and SIGLEC-1.

Embodiment 36. The antibody or antigen-binding fragment of any one of Embodiments 1-35, which is capable of inhibiting an interaction between SARS-CoV-2 and any one or more of: DC-SIGN; L-SIGN; SIGLEC-1; CD22; CD33; CLEC4M, SIGLEC-16; SIGLEC-15; SIGLEC-14; SIGLEC-12; SIGLEC-11; SIGLEC-10; SIGLEC-9; SIGLEC-8; SIGLEC-7; SIGLEC-6; SIGLEC-5; or any combination thereof.

Embodiment 37. The antibody or antigen-binding fragment of any one of Embodiments 1-36, which is capable of binding to a surface glycoprotein of any one or more of (i)-(viii): (i) one or more sarbecovirus of Clade 1a, wherein the one or more sarbecovirus optionally comprises SARS-CoV, Rs3367, Rs4084, LYRa3, Rs4231, Rs4874, WIV1, or any combination thereof; (ii) one or more sarbecovirus of Clade 1b, wherein the one or more sarbecovirus comprises SARS-CoV-2 and, optionally, one or more of RatG13, PangGD, and PangGX; (iii) one or more sarbecovirus of Clade 2, wherein the one or more sarbecovirus comprises Rml/2004, As6526, HKU3-12, Rp/Shaanxi2011, Cp/Yunnan2011, Rf4092, Rs4255, ZXC21, ZC45, YN2013, RMYN02, SC2018, Anlong1 12, YN2013, SC2011, ZC45, or any combination thereof, (iv) one or more sarbecovirus of Clade 3, wherein the one or more sarbecovirus optionally comprises BtKY72, BGR2008, or both; (iv) a variant of SEQ ID NO.:3 comprising: (iv)(a) a N501Y mutation; (iv)(b) a Y453F mutation; (iv)(c) a N439K mutation; (iv)(d) a K417V mutation; and/or (iv)(e) a N501Y mutation, a K417N mutation, and a E484K mutation; (v) a SARS-CoV-2 B.1.351 variant; (vi) a SARS-CoV-2 B.1.429 variant; (vii) a SARS-CoV-2 P.1 variant; (viii) a SARS-CoV-2 B.1.1.222 variant.

Embodiment 38. The antibody or antigen-binding fragment of any one of Embodiments 1-37, which is capable of binding to a surface glycoprotein of:
(i) a SARS-CoV-2 Wuhan-Hu-1 (SEQ ID NO.:3); (ii) a SARS-CoV-2 B.1.1.7; (iii) a SARS-CoV-2 B.1.351; (iv) a SARS-CoV-2 variant P.1; (v) a SARS-CoV-2 variant B.1.429; (vi) a SARS-CoV; (vii) a WIV1; (viii) a PANG/GD; (ix) a PANG/GX; (x) a RatG13; (xi) a ZXC21; (xii) a ZC45; (xiii) a RmYN02; (xiv) a BGR2008; (xv) a BtkY72; or (xvi) any combination of (i)-(xv).

Embodiment 39. The antibody or antigen-binding fragment of any one of Embodiments 1-38, wherein a Fab of the antibody or antigen-binding fragment is capable of binding to a SARS-CoV-2 S protein trimer with a Kd of less than 0.1 nM, optionally using surface plasmon resonance, further optionally measuring binding of captured S protein trimer to the Fab at 11, 33, 100, and 300 nM in single-cycle kinetics format.

Embodiment 40. The antibody or antigen binding fragment of any one of Embodiments 1-39, wherein a Fab of the antibody or antigen-binding fragment is capable of binding to a SARS-CoV-2 RBD with a Kd of less than 0.1 nM, optionally 0.08 nM, further optionally using surface plasmon resonance, further optionally measuring binding of captured RBD to the Fab at 11, 33, 100, and 300 nM in single-cycle kinetics format.

Embodiment 41. The antibody or antigen binding fragment of any one of Embodiments 1-40, wherein a Fab of the antibody or antigen-binding fragment is capable of binding to: (i) a Pangolin-GX RBD with a Kd of between 9 and 11 nM, optionally 10 nM; (ii) a SARS-CoV RBD with a Kd of between 1.5 nM and 1.7 nM, optionally 1.6 nM; (iii) a RaTG13 RBD with a Kd of between 1.0 nM and 1.2 nM, optionally 1.1 nM; (iv) a Pangolin-GD RBD with a Kd of between 0.1 nM and 0.3 nM, optionally 0.2 nM; (v) a WIV1 RBD with a Kd of between 1.3 nM and 1.5 nM, optionally 1.4 nM; (vi) a SC2018 RBD with a Kd of between 63 nM and 65 nM, optionally 64 nM; (vii) a ZC45 RBD with a Kd of between 3 nM and 5 nM, optionally 4 nM; (viii) a BTKY72 RBD with a Kd of between 0.3 nM and 0.5 nM, optionally 0.4 nM; and/or (ix) a BGR/2008 RBD with Kd of between 3.5 nM and 3.7 nM, optionally 3.6 nM, wherein binding is optionally measured using surface plasmon resonance, further optionally measuring binding of captured RBD to the Fab at 11, 33, 100, and 300 nM in single-cycle kinetics format.

Embodiment 42. The antibody or antigen binding fragment of any one of Embodiments 1-41, which is capable of neutralizing an infection by a SARS-CoV-2 and optionally one or more sarbecovirus that is not a SARS-CoV-2.

Embodiment 43. The antibody or antigen binding fragment of any one of Embodiments 1-42, which is capable of neutralizing in vitro infection by a SARS-CoV-2 pseudovirus, optionally a murine leukemia virus pseudotyped with SARS-CoV-2 S protein, with an IC50 of about 31.6 ng/mL, an IC80 of about 58.7 ng/mL, and/or an IC90 of about 87 ng/mL.

Embodiment 44. The antibody or antigen binding fragment of any one of Embodiments 1-43, which is capable of neutralizing in vitro infection by a SARS-CoV-2 pseudovirus, optionally a murine leukemia virus pseudotyped with SARS-CoV-2 S protein, with an IC50 between 85 ng/mL and 95 ng/mL, optionally between 91 ng/mL and 93 ng/mL or between 85 ng/mL and 88 ng/mL, an IC80 of about 184.5 ng/mL, and/or an IC90 of about 274 ng/mL.

Embodiment 45. The antibody or antigen binding fragment of any one of Embodiments 1-44, which is capable of neutralizing infection by a live SARS-CoV-2, optionally with an IC50 of between 140 ng/mL and 150 ng/mL, further optionally with an IC50 of between 142 ng/mL and 146 ng/mL.

Embodiment 46. The antibody or antigen binding fragment of any one of Embodiments 1-45, which is capable of neutralizing infection by: (i) a vesicular stomatitis virus (VSV) pseudotyped with SARS-CoV-2 S protein, optionally with an IC50 of between 210 ng/mL and 215 ng/mL, further optionally between 212 ng/mL and 214 ng/mL; (ii) a VSV pseudotyped with a B.1.1.7 S protein, with an IC50 of between 200 and 210 ng/mL, optionally between 203 ng/mL and 207 ng/mL; (iii) a VSV pseudotyped with a B.1.351 S protein, optionally with an IC50 of between 110 ng/mL and 120 ng/mL, further optionally between 112 ng/mL and 116 ng/mL; (iv) a VSV pseudotyped with a B.1.429 S protein, optionally with an IC50 of between 350 ng/mL and 360 ng/mL, further optionally between 355 ng/mL and 359 ng/mL; (v) a VSV pseudotyped with a P.1 S protein, optionally with an IC50 of between 450 ng/mL and 470 ng/mL, further optionally between 455 ng/mL and 465 ng/mL; (vi) a VSV pseudotyped with a SARS-CoV-2 S protein comprising a N439K mutation, optionally with an IC50 of between 270 ng/mL and 290 ng/mL, further optionally between 275 ng/mL and 285 ng/mL; (vii) a VSV pseudotyped with a SARS-CoV-2 S protein comprising a Y453F mutation, optionally with an IC50 of between 210 ng/mL and 230 ng/mL, further optionally between 215 ng/mL and 225 ng/mL; (viii) a VSV pseudotyped with a SARS-CoV S protein, optionally with an IC50 of between 80 ng/mL and 100 ng/mL, further optionally between 85 ng/mL and 90 ng/mL; (ix) a VSV pseudotyped with a RsSHC014 S protein, optionally with an IC50 of between 5 ng/mL and 10 ng/mL, further optionally between 6 ng/mL and 8 ng/mL; (x) a VSV pseudotyped with a WIV1 S protein, optionally with an IC50 of between 570 ng/mL and 590 ng/mL, further optionally between 575 ng/mL and 585 ng/mL; (xi) a VSV pseudotyped with a WIV16 S protein, optionally with an IC50 of between 190 ng/mL and 200 ng/mL, further optionally between 193 ng/mL and 199 ng/mL; (xii) a VSV pseudotyped with a RaTG-13 S protein, optionally with an IC50 of between 30 ng/mL and 45 ng/mL, further optionally between 35 ng/mL and 42 ng/mL; (xiii) a VSV pseudotyped with a Pangolin-GX S protein, optionally with an IC50 of between 4800 ng/mL and 4900 ng/mL, further optionally between 4840 ng/mL and 4870 ng/mL, further optionally between 4850 ng/mL and 4860 ng/mL; and/or (xiv) a VSV pseudotyped with a Pangolin-GD S protein, optionally with an IC50 of between 100 ng/mL and 110 ng/mL, further optionally between 103 ng/mL and 109 ng/mL.

Embodiment 47. The antibody or antigen binding fragment of any one of Embodiments 1-46, which is capable of inhibiting binding of a SARS-CoV-2 S protein RBD to human ACE2, optionally as measured by ELISA, further optionally with an IC50 of between 22 ng/mL and 28 ng/mL, optionally still further optionally between 22 ng/mL and 23 ng/mL or between 27 ng/mL and 28 ng/mL.

Embodiment 48. The antibody or antigen-binding fragment of any one of Embodiments 1-47, which is capable of inhibiting binding of a SARS-CoV-2 S protein RBD to human ACE2, optionally as measured by ELISA, further optionally with an IC50 of between 9 ng/mL and 11 ng/mL.

Embodiment 49. The antibody or antigen-binding fragment of any one of Embodiments 1-48, which is capable of inhibiting shedding of a SARS-CoV-2 S protein S1 subunit from by a cell infected with the SARS-CoV-2.

Embodiment 50. The antibody or antigen-binding fragment of any one of Embodiments 1-49, which is capable of preventing cell-cell fusion between cells expressing a SARS-CoV-2 S protein.

Embodiment 51. The antibody or antigen-binding fragment of any one of Embodiments 1-50, which is a IgG, IgA, IgM, IgE, or IgD isotype.

Embodiment 52. The antibody or antigen-binding fragment of any one of Embodiments 1-51, which is an IgG isotype selected from IgG1, IgG2, IgG3, and IgG4.

Embodiment 53. The antibody or antigen-binding fragment of any one of Embodiments 1-52, which is human, humanized, or chimeric.

Embodiment 54. The antibody or antigen-binding fragment of any one of Embodiments 1-53, wherein the antibody, or the antigen-binding fragment, comprises a human antibody, a monoclonal antibody, a purified antibody, a single chain antibody, a Fab, a Fab′, a F(ab′)2, a Fv, a scFv, or a scFab.

Embodiment 55. The antibody or antigen-binding fragment of Embodiment 54, wherein the antibody or antigen-binding fragment comprises a scFv comprising more than one VH domain and more than one VL domain.

Embodiment 56. The antibody or antigen-binding fragment of any one of Embodiments 1-55, wherein the antibody or antigen-binding fragment is a multi-specific antibody or antigen binding fragment.

Embodiment 57. The antibody or antigen-binding fragment of Embodiment 56, wherein the antibody or antigen binding fragment is a bispecific antibody or antigen-binding fragment.

Embodiment 58. The antibody or antigen-binding fragment of Embodiment 56 or 57, comprising:
(i) a first VH and a first VL; and
(ii) a second VH and a second VL,
wherein the first VH and the second VH are different and each independently comprise an amino acid sequence having at least 85% identity to the amino acid sequence set forth in any one of SEQ ID NOs.: 22, 30, 32, 34, 35, 37, 45, 47, 49, 50, 52, 54, 62, 64, 66, 68, 69, 71, 81, 91, 101, 111, 121, 135, 145, 155, 180, 190, 200, 210, 220, 233, 243, 252, 253, 255, 256, 258, 260, 262, 264, 266, 267, 270, 272, 274, 276, 278, 280, 284, 286, 288, 291, 292, 295, 297, 298, 300, 304, 314, 348, 358, 368, 378, 388, 398, 408, 418, 428, 432, 434, 437, 446, 448, 458, 459, and 460, and
wherein the first VL and the second VL are different and each independently comprise an amino acid sequence having at least 85% identity to the amino acid sequence set forth in any one of SEQ ID NOs.: 26, 41, 58, 75, 85, 95, 105, 115, 125, 139, 149, 184, 194, 204, 214, 224, 230, 236, 246, 282, 302, 308, 319, 352, 362, 372, 382, 392, 402, 412, 422, 439, 442, 443, 444, and 445;
and wherein the first VH and the first VL together form a first antigen-binding site, and wherein the second VH and the second VL together form a second antigen-binding site.

Embodiment 59. The antibody or antigen-binding fragment of any one of Embodiments 1-58, wherein the antibody or antigen-binding fragment further comprises a Fc polypeptide or a fragment thereof.

Embodiment 60. The antibody or antigen-binding fragment of Embodiment 59, wherein the Fc polypeptide or fragment thereof comprises:
(i) a mutation that enhances binding to a FcRn as compared to a reference Fc polypeptide that does not comprise the mutation; and/or
(ii) a mutation that enhances binding to a FcγR as compared to a reference Fc polypeptide that does not comprise the mutation.

Embodiment 61. The antibody or antigen-binding fragment of Embodiment 60, wherein the mutation that enhances binding to a FcRn comprises: M428L; N434S; N434H; N434A; N434S; M252Y; S254T; T256E; T250Q; P2571; Q31 11; D376V; T307A; E380A; or any combination thereof.

Embodiment 62. The antibody or antigen-binding fragment of Embodiment 60 or 61, wherein the mutation that enhances binding to FcRn comprises:
(i) M428L/N434S; (ii) M252Y/S254T/T256E; (iii) T250Q/M428L; (iv) P257I/Q311I; (v) P257I/N434H; (vi) D376V/N434H; (vii) T307A/E380A/N434A; or (viii) any combination of (i)-(vii).

Embodiment 63. The antibody or antigen-binding fragment of any one of Embodiments 60-62, wherein the mutation that enhances binding to FcRn comprises M428L/N434S.

Embodiment 64. The antibody or antigen-binding fragment of any one of Embodiments 60-63, wherein the mutation that enhances binding to a FcγR comprises S239D; 1332E; A330L; G236A; or any combination thereof.

Embodiment 65. The antibody or antigen-binding fragment of any one of Embodiments 60-64, wherein the mutation that enhances binding to a FcγR comprises: (i) S239D/I332E; (ii) S239D/A330L/I332E; (iii) G236A/S239D/I332E; or (iv) G236A/A330L/I332E.

Embodiment 66. The antibody or antigen-binding fragment of any one of Embodiments 1-65, which comprises a mutation that alters glycosylation, wherein the mutation that alters glycosylation comprises N297A, N297Q, or N297G, and/or which is aglycosylated and/or afucosylated.

Embodiment 67. The antibody or antigen-binding fragment of any one of Embodiments 1-66, which is capable of activating a human FcγRIIa, a human FcγRIIIa, or both, when bound to a SARS-CoV-2 S protein expressed on a surface of a target cell, wherein, optionally:
(i) the target cell comprises an EpiCHO cell;
(ii) the human FcγRIIa comprises a H131 allele;
(iii) the human FcγRIIIa comprises a V158 allele; and/or
(iv) the human FcγRIIIa and/or the human FcγRIIa is expressed by a host cell, such as a Jurkat cell or a Natural Killer cell, and activation is determined using a NFAT-driven luciferase signal in the host cell.

Embodiment 68. The antibody or antigen-binding fragment of any one of Embodiments 1-67, wherein the antibody or antigen-binding fragment is capable of inducing antibody-dependent cell-mediated cytotoxicity (ADCC) and/or antibody dependent cellular phagocytosis (ADCP) against a target cell infected by SARS-CoV-2.

Embodiment 69. The antibody or antigen-binding fragment of any one of Embodiments 59-68, wherein the Fc polypeptide comprises a L234A mutation and a L235A mutation.

Embodiment 70. The antibody or antigen-binding fragment of any one of Embodiments 1-69, wherein the antibody or antigen-binding fragment is capable of binding to a SARS-CoV-2 S protein, as determined using biolayer interferometry.

Embodiment 71. The antibody or antigen-binding fragment of Embodiments 1-70, wherein the antibody or antigen-binding fragment is capable of neutralizing a SARS-CoV-2 infection and/or of neutralizing an infection of a target cell with an IC50 of about 16 to about 20 μg/ml.

Embodiment 72. The antibody or antigen-binding fragment of Embodiments 1-71, wherein the antibody or antigen-binding fragment is capable of neutralizing a SARS-CoV-2 infection and/or of neutralizing an infection of a target cell with an IC50 of about 3 to about 4 μg/ml.

Embodiment 73. The antibody or antigen-binding fragment of any one of Embodiments 1-72, wherein the antibody or antigen-binding fragment is capable of neutralizing a SARS-CoV-2 infection and/or of neutralizing an infection of a target cell with an IC50 of about 0.8 to about 0.9 μg/ml.

Embodiment 74. The antibody or antigen-binding fragment of any one of Embodiments 1-73, wherein the antibody or antigen-binding fragment is capable of neutralizing a SARS-CoV-2 infection and/or of neutralizing an infection of a target cell with an IC50 of about 0.5 to about 0.6 μg/ml.

Embodiment 75. The antibody or antigen-binding fragment of any one of Embodiments 1-74, wherein the antibody or antigen-binding fragment is capable of neutralizing a SARS-CoV-2 infection and/or of neutralizing an infection of a target cell with an IC50 of about 0.1 to about 0.2 μg/ml.

Embodiment 76. An isolated polynucleotide encoding the antibody or antigen-binding fragment of any one of Embodiments 1-75, or encoding a VH, a heavy chain, a VL, and/or a light chain of the antibody or the antigen-binding fragment.

Embodiment 77. The polynucleotide of Embodiment 76, wherein the polynucleotide comprises deoxyribonucleic acid (DNA) or ribonucleic acid (RNA), wherein the RNA optionally comprises messenger RNA (mRNA).

Embodiment 78. The polynucleotide of Embodiment 76 or 77, which is codon-optimized for expression in a host cell.

Embodiment 79. The polynucleotide of any one of Embodiments 76-78, comprising a polynucleotide having at least 50% identity to the polynucleotide sequence according to any one or more of SEQ ID NOs.: 79, 80, 89, 90, 99, 100, 109, 110, 119, 120, 129-134, 143, 144, 153, 154, 157, 159, 188, 189, 198, 199, 208, 209, 218, 219, 228, 229, 231, 240, 241, 250, 251, 312, 313, 322, 323, 356, 357, 366, 367, 376, 377, 386, 387, 396, 397, 406, 407, 416, 417, 426, 427, 429, 430, 431, 433, 436, 438, and 441.

Embodiment 80. A recombinant vector comprising the polynucleotide of any one of Embodiments 76-79.

Embodiment 81. A host cell comprising the polynucleotide of any one of Embodiments 76-79 and/or the vector of Embodiment 80, wherein the polynucleotide is heterologous to the host cell.

Embodiment 82. A human B cell comprising the polynucleotide of any one of Embodiments 76-79, wherein polynucleotide is heterologous to the human B cell and/or wherein the human B cell is immortalized.

Embodiment 83. A composition comprising:
(i) the antibody or antigen-binding fragment of any one of Embodiments 1-75; (ii) the polynucleotide of any one of Embodiments 76-79; (iii) the recombinant vector of Embodiment 80; (iv) the host cell of Embodiment 81; and/or (v) the human B cell of Embodiment 82; and a pharmaceutically acceptable excipient, carrier, or diluent.

Embodiment 84. The composition of Embodiment 83, comprising two or more different antibodies or antigen-binding fragments, wherein each of the two or more different antibodies or antigen-binding fragments is different and is independently according to of any one of Embodiments 1-75.

Embodiment 85. The composition of Embodiment 83 or 84, further comprising an antibody or antigen-binding fragment comprising CDRH1, CDRH2, CDRH3, CDRL1, CDRL2, and CDRL3 amino acid sequences as set forth in SEQ ID NOs.:173, 174, 175, 177, 178, and 179, respectively, and optionally comprising the VH amino acid sequence of SEQ ID NO.:172 and the VL amino acid sequence of SEQ ID NO.:176.

Embodiment 86. The composition of Embodiment 83 or 84, further comprising an antibody or antigen-binding fragment comprising CDRH1, CDRH2, CDRH3, CDRL1, CDRL2, and CDRL3 amino acid sequences as set forth in SEQ ID NOs.:341, 342, 343, 345, 346, and 347, respectively, and optionally comprising the VH amino acid sequence of SEQ ID NO.:340 and the VL amino acid sequence of SEQ ID NO.:344.

Embodiment 87. The composition of any one of Embodiments 83-86, comprising the antibody or antigen-binding fragment of any one of Embodiments 5-11.

Embodiment 88. The composition of any one of Embodiments 83-87, comprising the antibody or antigen-binding fragment of any one of Embodiments 19-23.

Embodiment 89. The composition of any one of Embodiments 83-88, comprising the antibody or antigen-binding fragment of any one of Embodiments 12-18.

Embodiment 90. The composition of any one of Embodiments 83-89, comprising the antibody or antigen-binding fragment of any one of Embodiments 24 or 25.

Embodiment 91. The composition of any one of Embodiments 83-90, comprising: (i) a first antibody or antigen-binding fragment, comprising a VH comprising or consisting of the amino acid sequence as set forth in SEQ ID NO: 172 and a VL comprising or consisting of the amino acid sequence as set forth in SEQ ID NO: 176; and (ii) a second antibody or antigen-binding fragment comprising, a VH comprising or consisting of the amino acid sequence as set forth in any one of SEQ ID NOs: 22, 30, 32, 34, 35, 37, 45, 47, 49, 50, 52, 54, 62, 64, 66, 68, or 69 and a VL comprising of consisting of the amino acid sequence as set forth in any one of SEQ ID NOs: 26, 41, or 58.

Embodiment 92. The composition of any one of Embodiments 83-91, comprising: (i) a first antibody or antigen-binding fragment comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 173-175, respectively, and the CDRL1, CDRL2, and CDRL3 comprise or consist of the amino acid sequences set forth in SEQ ID NOs: 177-179, respectively; and (ii) a second antibody or antigen-binding fragment comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in (i) SEQ ID NOs: 23-25, respectively, (ii) SEQ ID NOs: 160-162, respectively, (iii) SEQ ID NOs: 38-40, respectively, or (iv) SEQ ID NOs: 166-168, respectively, and the CDRL1, CDRL2, and CDRL3 comprise or consist of the amino acid sequences set forth in (i) SEQ ID NOs: 27-29, respectively, (ii) SEQ ID NOs: 163-165, respectively, (iii) SEQ ID NOs: 42-44, respectively, or (iv) SEQ ID NOs: 169-171, respectively.

Embodiment 93. The composition of any one of Embodiment 83-92, comprising: (i) a first antibody or antigen-binding fragment, comprising a VH comprising or consisting of the amino acid sequence as set forth in SEQ ID NO: 172 and a VL comprising or consisting of the amino acid sequence as set forth in SEQ ID NO: 176; and (ii) a second antibody or antigen-binding fragment comprising, a VH comprising or consisting of the amino acid sequence as set forth in SEQ ID NO: 200 and a VL comprising of consisting of the amino acid sequence as set forth in SEQ ID NO: 204.

Embodiment 94. The composition of any one of Embodiments 83-93, comprising: (i) a first antibody or antigen-binding fragment comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 173-175, respectively, and the CDRL1, CDRL2, and CDRL3 comprise or consist of the amino acid sequences set forth in SEQ ID NOs: 177-179, respectively; and (ii) a second antibody or antigen-binding fragment comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs: 201-203, respectively, and the CDRL1, CDRL2, and CDRL3 comprise or consist of the amino acid sequences set forth in SEQ ID NOs: 205-207, respectively.

Embodiment 95. A composition comprising the polynucleotide of any one of Embodiments 76-79 encapsulated in a carrier molecule, wherein the carrier molecule optionally comprises a lipid, a lipid-derived delivery vehicle, such as a liposome, a solid lipid nanoparticle, an oily suspension, a submicron lipid emulsion, a lipid microbubble, an inverse lipid micelle, a cochlear liposome, a lipid microtubule, a lipid microcylinder, lipid nanoparticle (LNP), or a nanoscale platform.

Embodiment 96. A method of treating a sarbecovirus infection in a subject, the method comprising administering to the subject an effective amount of (i) the antibody or antigen-binding fragment of any one of Embodiments 1-75; (ii) the polynucleotide of any one of Embodiments 76-79; (iii) the recombinant vector of Embodiment 80; (iv) the host cell of Embodiment 81; (v) the human B cell of Embodiment 82; and/or (vi) the composition of any one of Embodiments 83-95.

Embodiment 97. The antibody or antigen-binding fragment of any one of Embodiments 1-75, the polynucleotide of any one of Embodiments 76-79, the recombinant vector of Embodiment 80, the host cell of Embodiment 81, the human B cell of Embodiment 82, and/or the composition of any one of Embodiments 83-95 for use in a method of treating a sarbecovirus infection in a subject.

Embodiment 98. The antibody or antigen-binding fragment of any one of Embodiments 1-75, the polynucleotide of any one of Embodiments 76-79, the recombinant vector of Embodiment 80, the host cell of Embodiment 81, the human B cell of Embodiment 82, and/or the composition of any one of Embodiments 83-95 for use in the preparation of a medicament for the treatment of a SARS-CoV-2 infection in a subject.

Embodiment 99. The method of Embodiment 96, or the antibody, antigen-binding fragment, polynucleotide, recombinant vector, host cell, human B cell, and/or composition for use of Embodiment 97 or 98, wherein the antibody or antigen-binding fragment is according to any one of Embodiments 5-11.

Embodiment 100. The method of Embodiment 96 or 99, or the antibody, antigen-binding fragment, polynucleotide, recombinant vector, host cell, human B cell, and/or composition for use of any one of Embodiments 97-99, wherein the antibody or antigen-binding fragment is according to any one of Embodiments 19-23.

Embodiment 101. The method of Embodiment 96 or the antibody, antigen-binding fragment, polynucleotide, recombinant vector, host cell, human B cell, and/or composition for use of Embodiment 97 or 98, wherein the antibody or antigen-binding fragment is according to any one of Embodiments 12-18.

Embodiment 102. The method of Embodiment 96 or 99, or the antibody, antigen-binding fragment, polynucleotide, recombinant vector, host cell, human B cell, and/or composition for use of any one of Embodiments 97-99, wherein the antibody or antigen-binding fragment is according to any one of Embodiments 24 or 25.

Embodiment 103. The method of any one of Embodiments 96-102 or the antibody, antigen-binding fragment, polynucleotide, recombinant vector, host cell, human B cell, and/or composition for use of any one of Embodiments 97-102, wherein the method further comprises administering and/or wherein the subject has received an antibody or antigen-binding fragment comprising CDRH1, CDRH2, CDRH3, CDRL1, CDRL2, and CDRL3 amino acid sequences as set forth in SEQ ID NOs.:173, 174, 175, 177, 178, and 179, respectively, and optionally comprising the VH amino acid sequence of SEQ ID NO.:172 and the VL amino acid sequence of SEQ ID NO.:176.

Embodiment 104. The method of any one of Embodiments 96-103 or the antibody, antigen-binding fragment, polynucleotide, recombinant vector, host cell, human B cell, and/or composition for use of any one of Embodiments 97-103, wherein the method further comprises administering and/or wherein the subject has received an antibody or antigen-binding fragment comprising CDRH1, CDRH2, CDRH3, CDRL1, CDRL2, and CDRL3 amino acid sequences as set forth in SEQ ID NOs.:341, 342, 343, 345, 346, and 347, respectively, and optionally comprising the VH amino acid sequence of SEQ ID NO.:340 and the VL amino acid sequence of SEQ ID NO.:344.

Embodiment 105. The method of any one of Embodiments 96-104 or the antibody, antigen-binding fragment, polynucleotide, recombinant vector, host cell, human B cell, and/or composition for use of any one of Embodiments 97-104, wherein the sarbecovirus comprises a sarbecovirus of Clade 1a, a sarbecovirus of clade 1b, a sarbecovirus of clade 2, and/or a sarbecovirus of clade 3.

Embodiment 106. The method of any one of Embodiments 96-105 or the antibody, antigen-binding fragment, polynucleotide, recombinant vector, host cell, human B cell, and/or composition for use of any one of Embodiments 97-105, wherein the sarbecovirus comprises a SARS-CoV-2.

Embodiment 107. A method for in vitro diagnosis of a SARS-CoV-2infection, the method comprising: (i) contacting a sample from a subject with an antibody or antigen-binding fragment of any one of Embodiments 1-75; and (ii) detecting a complex comprising an antigen and the antibody, or comprising an antigen and the antigen-binding fragment.

Embodiment 108. The method of Embodiment 107, wherein the sample comprises blood isolated from the subject.

Embodiment 109. A method of preventing or treating or neutralizing a coronavirus infection in a subject, the method comprising: administering to a subject who has received a first antibody or antigen binding fragment comprising: (a) VH and VL amino acid sequences according to SEQ ID NOs.:172 and 176 respectively; or (b) CDRH1, CDRH2, CDRH3, CDRL1, CDRL2, and CDRL3 amino acid sequences according to SEQ ID NOS.:173-175 and 177-179, respectively; and a second antibody or antigen binding fragment comprising: (a) a VH amino acid sequence according to any one of SEQ ID NOs.: 22, 30, 32, 34, 35, 37, 45, 47, 49, 50, 52, 54, 62, 64, 66, 68, or 69, and a VL amino acid sequence according to any one of SEQ ID NOs: 26, 41, or 58; or (b) CDRH1, CDRH2, and CDRH3 amino acids according to (i) SEQ ID NOs: 23-25, respectively, (ii) SEQ ID NOs: 160-162, respectively, (iii) SEQ ID NOs: 38-40, respectively, or (iv) SEQ ID NOs: 166-168, respectively, and CDRL1, CDRL2, and CDRL3 amino acid sequences according to (i) SEQ ID NOs: 27-29, respectively, (ii) SEQ ID NOs: 163-165, respectively, (iii) SEQ ID NOs: 42-44, respectively, or (iv) SEQ ID NOs: 169-171, respectively.

Embodiment 110. A method of preventing or treating or neutralizing a coronavirus infection in a subject, the method comprising: administering to a subject who has received a first antibody or antigen binding fragment comprising: (a) a VH amino acid sequence according to any one of SEQ ID NOs.: 22, 30, 32, 34, 35, 37, 45, 47, 49, 50, 52, 54, 62, 64, 66, 68, or 69, and a VL amino acid sequence according to any one of SEQ ID NOs: 26, 41, or 58; or (b) CDRH1, CDRH2, and CDRH3 amino acids according to (i) SEQ ID NOs: 23-25, respectively, (ii) SEQ ID NOs: 160-162, respectively, (iii) SEQ ID NOs: 38-40, respectively, or (iv) SEQ ID NOs: 166-168, respectively, and CDRL1, CDRL2, and CDRL3 amino acid sequences according to (i) SEQ ID NOs: 27-29, respectively, (ii) SEQ ID NOs: 163-165, respectively, (iii) SEQ ID NOs: 42-44, respectively, or (iv) SEQ ID NOs: 169-171, respectively; and a second antibody or antigen binding fragment comprising: (a) VH and VL amino acid sequences according to SEQ ID NOs.:172 and 176 respectively; or (b) CDRH1, CDRH2, CDRH3, CDRL1, CDRL2, and CDRL3 amino acid sequences according to SEQ ID NOS.:173-175 and 177-179, respectively.

Embodiment 111. A method of preventing or treating or neutralizing a coronavirus infection in a subject, the method comprising: administering to a subject who has received a first antibody or antigen binding fragment comprising: (a) VH and VL amino acid sequences according to SEQ ID NOs.:172 and 176 respectively; or (b) CDRH1, CDRH2, CDRH3, CDRL1, CDRL2, and CDRL3 amino acid sequences according to SEQ ID NOS.:173-175 and 177-179, respectively; and a second antibody or antigen binding fragment comprising: (a) a VH amino acid sequence according to SEQ ID NO.: 200 and a VL amino acid sequence according to SEQ ID NO: 204; or (b) CDRH1, CDRH2, and CDRH3 amino acids according to SEQ ID NOs: 201-203, respectively, and CDRL1, CDRL2, and CDRL3 amino acid sequences according to SEQ ID NOs: 205-207, respectively.

Embodiment 112. A method of preventing or treating or neutralizing a coronavirus infection in a subject, the method comprising: administering to a subject who has received a first antibody or antigen binding fragment comprising: (a) a VH amino acid sequence according to SEQ ID NO.: 200, and a VL amino acid sequence according to SEQ ID NO: 204; or (b)CDRH1, CDRH2, and CDRH3 amino acids according to SEQ ID NO: 201-203, respectively, and CDRL1, CDRL2, and CDRL3 amino acid sequences according to SEQ ID NO: 205-207, respectively; and a second antibody or antigen binding fragment comprising: (a) VH and VL amino acid sequences according to SEQ ID NOs.:172 and 176 respectively; or (b) CDRH1, CDRH2, CDRH3, CDRL1, CDRL2, and CDRL3 amino acid sequences according to SEQ ID NOS.:173-175 and 177-179, respectively.

Embodiment 113. A method of preventing or treating or neutralizing a sarbecovirus infection in a subject, the method comprising administering to the subject an effective amount of an antibody or an antigen-binding fragment that comprises CDRH1, CDRH2, CDRH3, CDRL1, CDRL2, and CDRL3 amino acid sequences as set forth in: (i) SEQ ID NOs.: 409, 410, 411, 413, 414, and 415, respectively; (ii) SEQ ID NOs.: 409, 447, 411, 413, 414, and 415, respectively; (iii) SEQ ID NOs.: 409, 457, 411, 413, 414, and 415, respectively; (iv) SEQ ID NOs.: 449, 410, 411, 413, 414, and 415, respectively; (v) SEQ ID NOs.: 449, 447, 411, 413, 414, and 415, respectively; or (vi) SEQ ID NOs.: 449, 457, 411, 413, 414, and 415, respectively, wherein the antibody or antigen-binding fragment is capable of binding to a SARS-CoV-2 surface glycoprotein (S).

Embodiment 114. A method of preventing or treating or neutralizing a sarbecovirus infection in a subject, the method comprising administering to the subject an effective amount of an antibody or an antigen-binding fragment that comprises CDRH1, CDRH2, CDRH3, CDRL1, CDRL2, and CDRL3 amino acid sequences as set forth in: (i) SEQ ID NOs.: 409, 410, 411, 413, 414, and 415, respectively; (ii) SEQ ID NOs.: 409, 447, 411, 413, 414, and 415, respectively; (iii) SEQ ID NOs.: 409, 457, 411, 413, 414, and 415, respectively; (iv) SEQ ID NOs.: 449, 410, 411, 413, 414, and 415, respectively; (v) SEQ ID NOs.: 449, 447, 411, 413, 414, and 415, respectively; or (vi) SEQ ID NOs.: 449, 457, 411, 413, 414, and 415, respectively, wherein the antibody or antigen-binding fragment is capable of binding to a SARS-CoV-2 surface glycoprotein (S).

Embodiment 115. The method of Embodiment 114, wherein the antibody or antigen-binding fragment comprises VH and VL amino acid sequences as set forth in: (a) 408 and 412, respectively; (b) 408 and 442, respectively; (c) 408 and 443, respectively; (d) 408 and 444, respectively; (e) 408 and 445, respectively; (f) 428 and 412, respectively; (g) 428 and 442, respectively; (h) 428 and 443, respectively; (i) 428 and 444, respectively; ( ) 428 and 445, respectively; (k) 446 and 412, respectively; (l) 446 and 442, respectively; (m) 446 and 443, respectively; (n) 446 and 444, respectively; (o) 446 and 445, respectively; (p) 448 and 412, respectively; (q) 448 and 442, respectively; (r) 448 and 443, respectively; (s) 448 and 444, respectively; (t) 448 and 445, respectively; (u) 458 and 412, respectively; (v) 458 and 442, respectively; (w) 458 and 443, respectively; (x) 458 and 444, respectively; (y) 458 and 445, respectively; (z) 459 and 412, respectively; (aa) 459 and 442, respectively; (bb) 459 and 443, respectively; (cc) 459 and 444, respectively; (dd) 459 and 445, respectively; (ee) 460 and 412, respectively; (ff) 460 and 442, respectively; (gg) 460 and 443, respectively; (hh) 460 and 444, respectively; or (ii) 460 and 445, respectively.

Embodiment 116. A method of preventing or treating or neutralizing a sarbecovirus infection in a subject, the method comprising administering to the subject an effective amount of an antibody or an antigen-binding fragment that comprises CDRH1, CDRH2, CDRH3, CDRL1, CDRL2, and CDRL3 amino acid sequences as set forth in: (i) SEQ ID NOs.: 399, 400, 401, 403, 404, and 405, respectively; (ii) SEQ ID NOs.: 399, 400, 435, 403, 404, and 405, respectively; (iii) SEQ ID NOs.: 399, 400, 401, 403, 440, and 405, respectively; or (iv) SEQ ID NOs.: 399, 400, 435, 403, 440, and 405, respectively, and wherein the antibody or antigen-binding fragment is capable of binding to a SARS-CoV-2 surface glycoprotein (S).

Embodiment 117. The method of Embodiment 116, wherein the antibody or antigen-binding fragment comprises VH and VL amino acid sequences as set forth in: (i) 398 and 402, respectively; (ii) 398 and 439, respectively; (iii) 432 and 402, respectively; (iv) 432 and 439, respectively; (v) 434 and 402, respectively; (vi) 434 and 439, respectively; (vii) 437 and 402, respectively; or (viii) 437 and 439, respectively, and wherein the antibody or antigen-binding fragment is capable of binding to a SARS-CoV-2 surface glycoprotein (S).

Embodiment 118. The method of any one of Embodiments 113-117, wherein the sarbecovirus comprises a sarbecovirus of Clade 1a, a sarbecovirus of clade 1b, a sarbecovirus of clade 2, and/or a sarbecovirus of clade 3, and optionally comprises a SARS-CoV-2.

Embodiment 119. The method of any one of Embodiments 113-118, wherein the method further comprises administering and/or wherein the subject has received an antibody or antigen-binding fragment comprising CDRH1, CDRH2, CDRH3, CDRL1, CDRL2, and CDRL3 amino acid sequences as set forth in SEQ ID NOs.:341, 342, 343, 345, 346, and 347, respectively, and optionally comprising the VH amino acid sequence of SEQ ID NO.:340 and the VL amino acid sequence of SEQ ID NO.:344.

Embodiment 120. The method of any one of Embodiments 113-119, wherein the method further comprises administering and/or wherein the subject has received an antibody or antigen-binding fragment comprising CDRH1, CDRH2, CDRH3, CDRL1, CDRL2, and CDRL3 amino acid sequences as set forth in SEQ ID NOs.:173, 174, 175, 177, 178, and 179, respectively, and optionally comprising the VH amino acid sequence of SEQ ID NO.:172 and the VL amino acid sequence of SEQ ID NO.:176.

### EXAMPLES

### Example 1

### Neutralization of SARS-CoV-2 by Recombinant Human Monoclonal Antibodies

Human monoclonal antibodies isolated from patients who recovered from SARS-CoV-2 infection were expressed recombinantly and were tested in neutralization assays against SARS-CoV-2 pseudotyped virus.

Murine leukemia virus (MLV) pseudotyped with SARS-CoV-2 Spike protein (SARS-CoV-2pp) were used. VeroE6 cells were used as target cells and were seeded one day before addition of virus and antibodies. SARS-CoV-2pp was activated with trypsin TPCK at 10 ug/ml. Activated SARS-CoV-2pp was added to a dilution series of antibodies and incubated for 48 hours. Starting concentration for antibodies was 5 ug/ml per antibody, 3-fold dilution. Luminescence was measured after aspirating cell culture supernatant and adding Bio-Glo substrate (Promega).

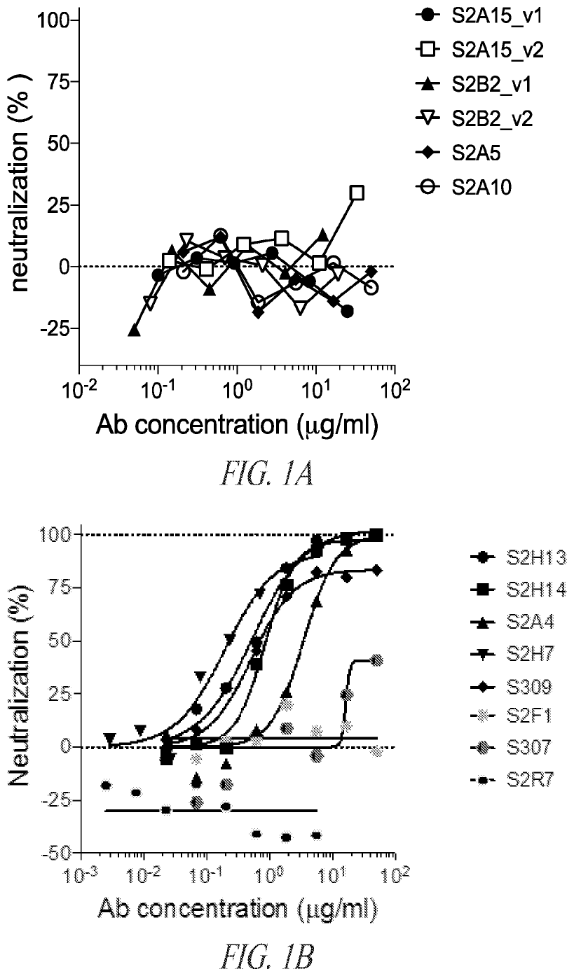

Results for antibodies S2A15-v1, S2A15-v2, S2B2-v1, S2B2-v2, S2A5, and S2A10 are shown in FIG. 1A. Results for antibodies S2H13, S2H14, S2A4, S2H7, S2F1, and S2R7 are shown in FIG. 1B, along with results for human monoclonal antibodies S307 and S309 (S309 VH SEQ ID NO.:172; S309 VL SEQ ID NO.:176), which were isolated from patients who recovered from SARS-CoV infection. IC50 values, in μg/ml, are shown in Table 4. FIG. 1C shows results for four antibodies from FIG. 1B that neutralized SARS-CoV-2pp.

TABLE 4

| | S2H13 | S2H14 | S2A4 | S2H7 | S309 | S2F1 | S307 | S2R7 |
|---|---|---|---|---|---|---|---|---|
| IC50 | 0.5343 | 0.8468 | 3.453 | 0.1949 | 0.5142 | — | approx. 16.18 | — |

Neutralization of infection by antibodies S2X2 and S309 using pseudotyped MLV was also measured. Data are shown in FIG. 8. Neutralization of SARS-CoV-2 pseudotyped MLV was assayed for each of S2X2 and S309, as well as for the combination of S2X2 and S309. Recombinant S2X2 and S309 antibodies with LS (M428L/N434S) Fc mutations were used. IC50 values, in ng/ml, are shown in Table 5.

TABLE 5

| | | | S309-rIgG1-LS + |
|---|---|---|---|
| | S309-rIgG1-LS | S2X2-rIgG1-LS | S2X2-rIgG1-LS |
| IC50 | 899.2 | 401.6 | 177.3 |

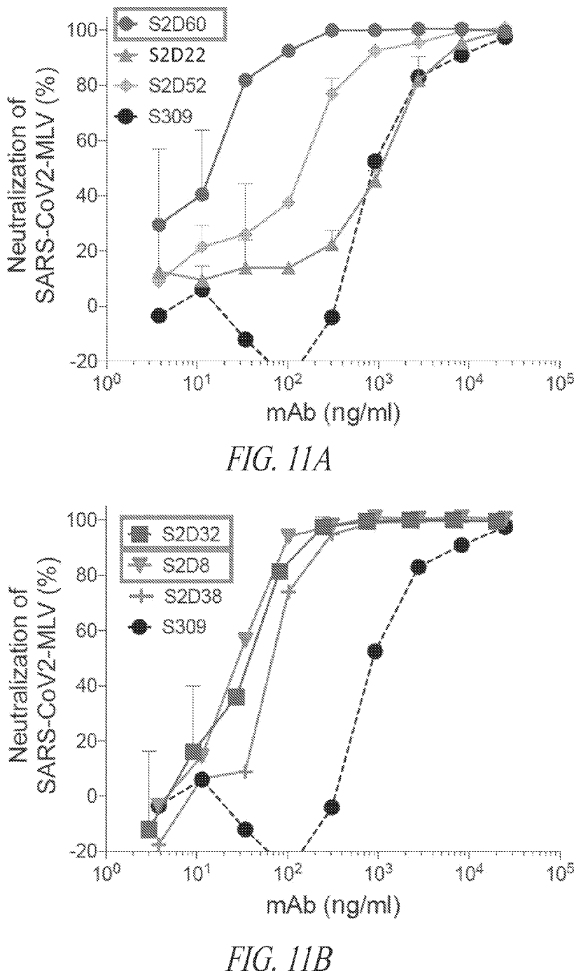

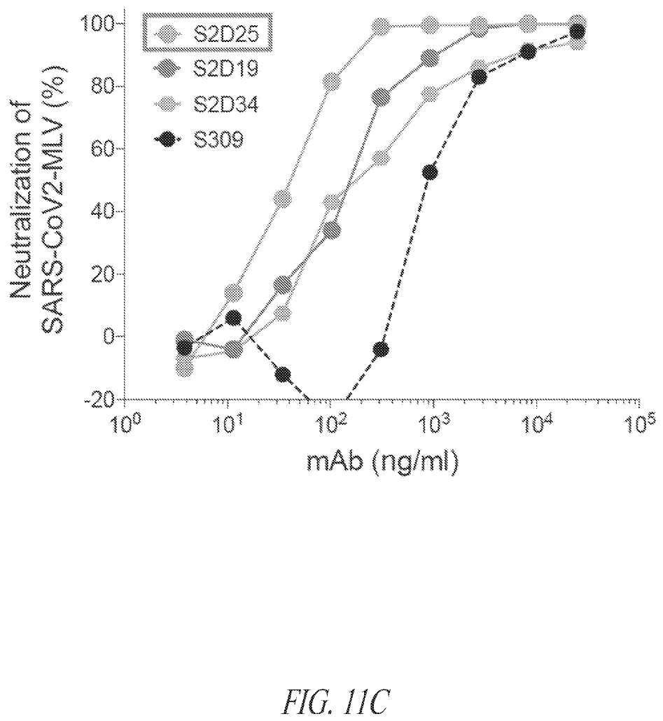

Neutralization of infection by antibodies S2D60, S2D22, S2D52, S2D32, S2D8, S2D38, S2D25, S2D19 S2D34, and S309 was assayed using similar methods. Results are shown in FIGS. 11A-11C. Neutralization IC values, in ng/ml, are shown in Table 6.

TABLE 6

| mAb | IC50 | IC80 | IC90 | IC95 |
|---|---|---|---|---|
| S2D60 | 12 | 40 | 84 | 164 |
| S2D8 | 30 | 58 | 87 | 125 |
| S2D25 | 40 | 91 | 146 | 228 |
| S2D32 | 36 | 81 | 129 | 200 |
| S2D38 | 73 | 117 | 154 | 200 |
| S2D52 | 108 | 510 | 1265 | 2919 |
| S2D19 | 150 | 405 | 723 | 1235 |
| S2D34 | 230 | 1014 | 2417 | 5378 |
| S2D22 | 865 | 3284 | 7164 | 14701 |
| S309 | 968 | 1699 | 2361 | 3197 |

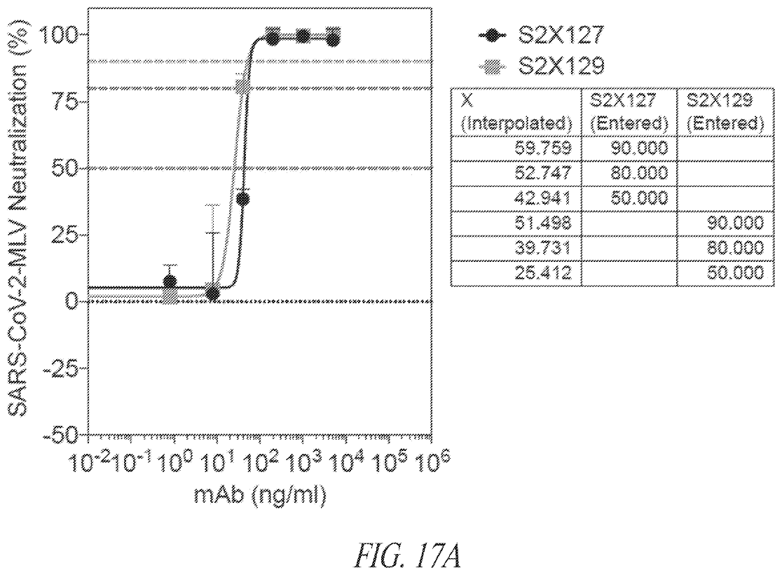

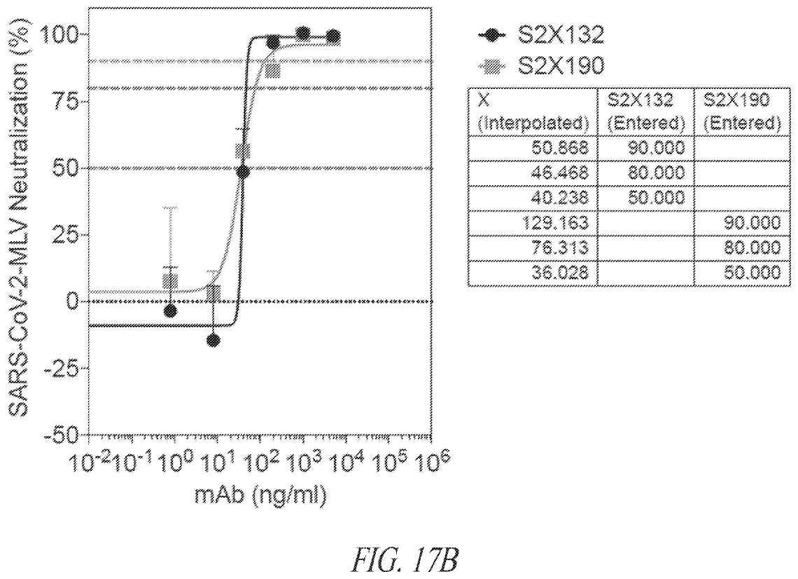

Neutralization of infection by antibodies S2X127, S2X129, S2X132, and S2X190 was assayed using similar methods. Results are shown in FIGS. 17A and 17B. Calculated IC50, IC80, and IC90 values are shown on the right of each figure.

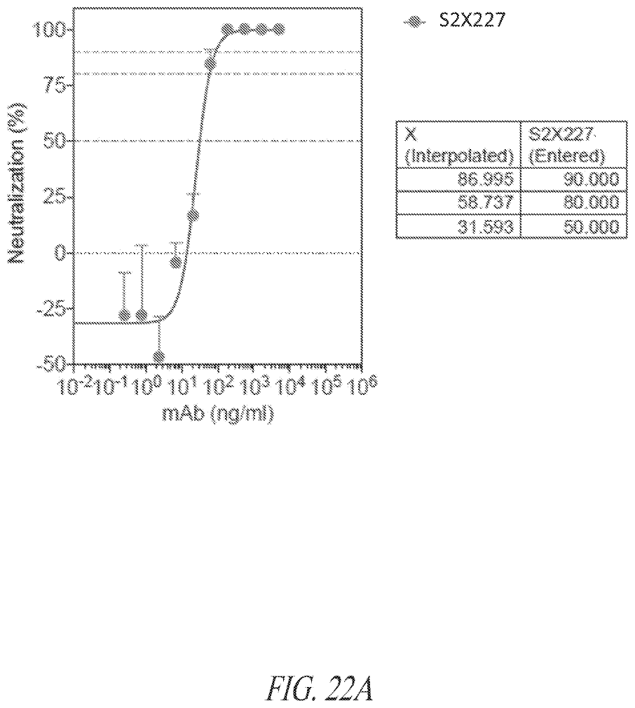

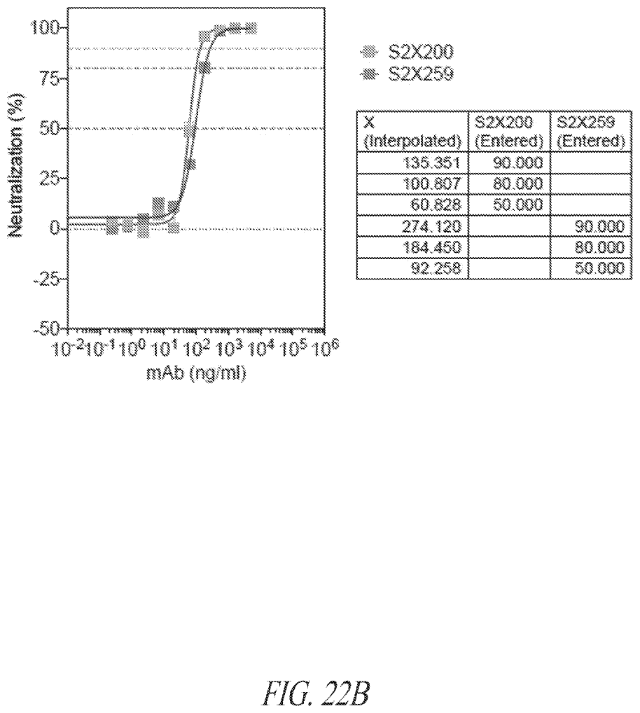

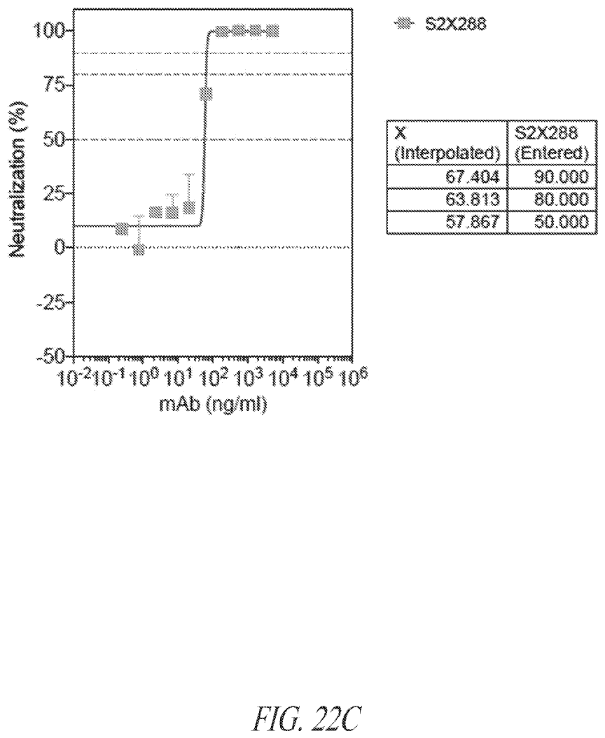

Neutralization of infection by antibodies S2X200, S2X259, S2X227 and S2X288 was assayed using similar methods. Results are shown in FIGS. 22A-22C. Calculated IC50, IC80, and IC90 values are shown in the box on the right side of each figure.

### Example 2

### Binding of Human Monoclonal Antibodies to SARS-Cov and SARS-CoV-2 RBD

Binding of human monoclonal antibodies isolated from patients who recovered from SARS-CoV-2 infection to the RBD of SARS-CoV and SARS-CoV-2 Spike protein was studied using enzyme-linked immunosorbent assay (ELISA).

96-well plates were coated with SARS-CoV-2 RBD (produced in house; residues 331-550 of spike from BetaCoV/Wuhan-Hu-1/2019, accession number MN908947), or SARS-CoV RBD (Sino Biological).

Wells were washed and blocked with PBS+1% BSA for 1 hour at room temperature and were then incubated with serially diluted recombinant monoclonal antibodies for 1 hour at room temperature. Bound antibodies were detected by incubating alkaline phosphatase-conjugated goat anti-human IgG (Southern Biotechnology: 2040-04) for 1 hour at room temperature and were developed by 1 mg/ml p-nitrophenylphosphate substrate in 0.1 M glycine buffer (pH 10.4) for 30 minutes at room temperature. The optical density (OD) values were measured at a wavelength of 405 nm in an ELISA reader (Powerwave 340/96 spectrophotometer, BioTek).

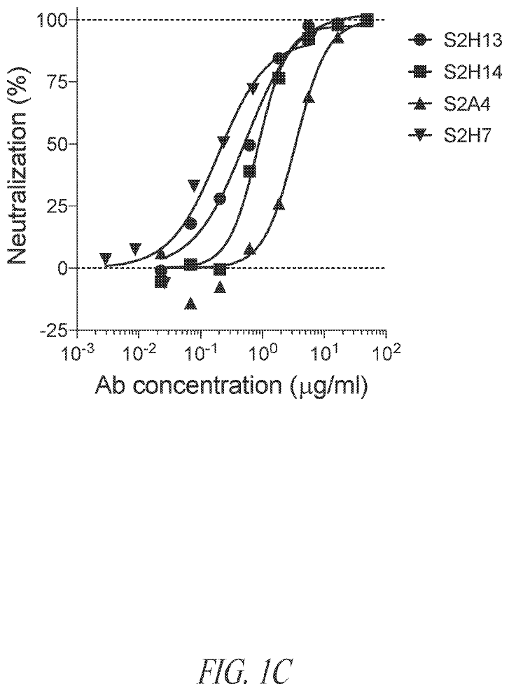

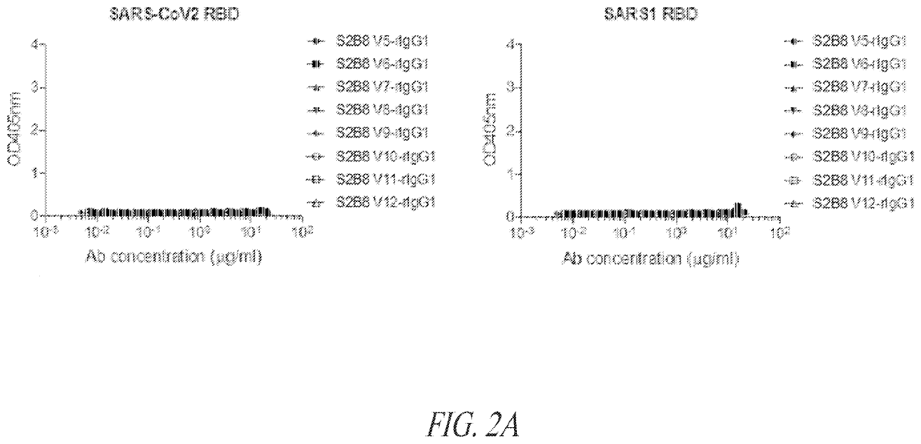

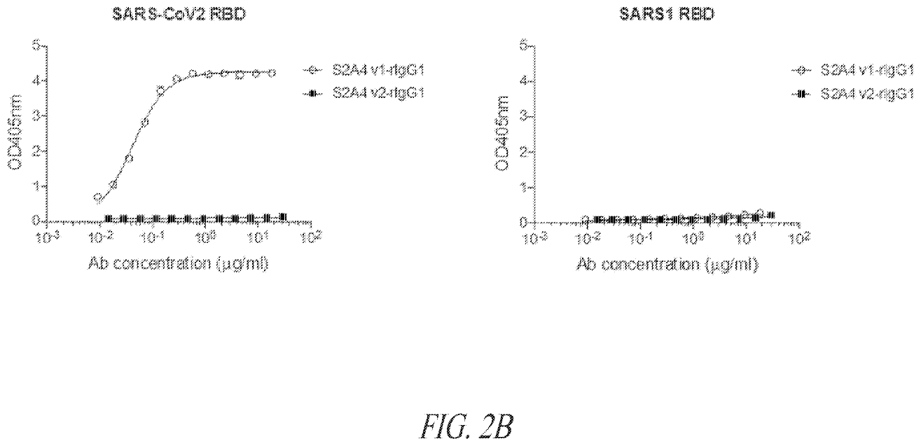

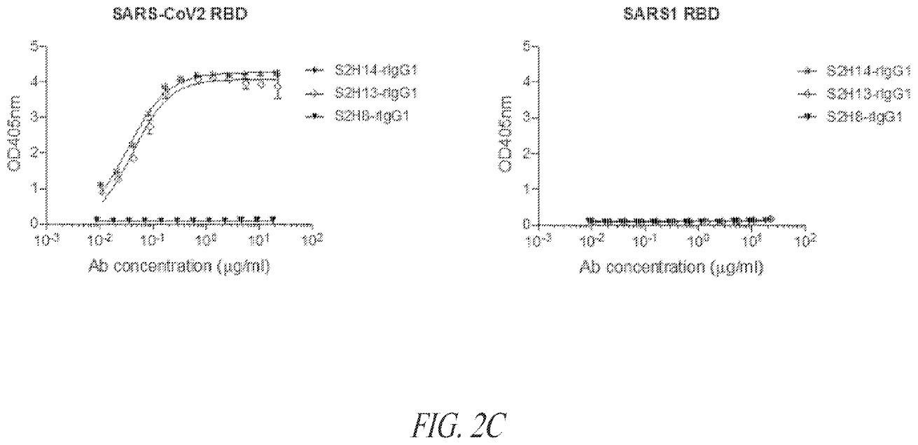

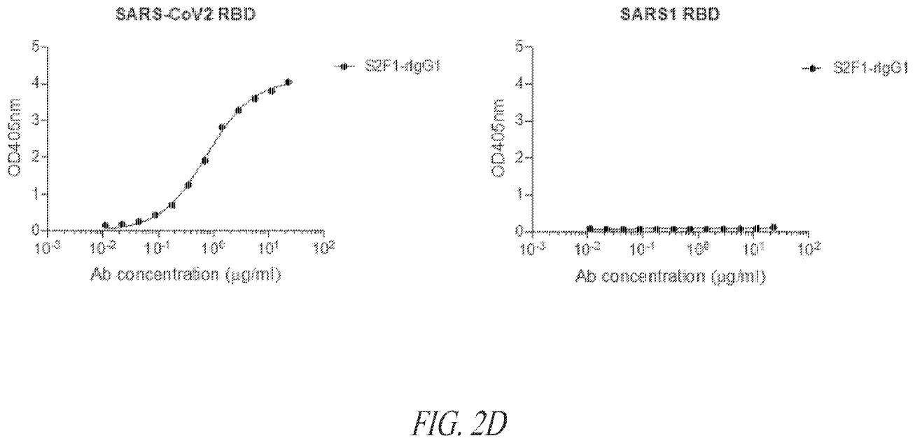

The ELISA assay results are shown in FIGS. 2A-2D. In each figure, binding to RBD of SARS-CoV-2 is shown in the left panel and binding to RBD of SARS-CoV is shown in the right panel.

### Example 3

### Binding of Human Monoclonal Antibodies to RBD of SARS-Cov and RBD and S1 Domain of SARS-CoV-2

Binding of human monoclonal antibodies isolated from patients who recovered from SARS-CoV-2 infection to the RBD of SARS-CoV and the RBD and S1 domain of SARS-CoV-2 was studied using enzyme-linked immunosorbent assay (ELISA).

96-well ELISA plates were coated with SARS-CoV RBD (Sino Biological, 40150-V08B1) at 1 μg/ml, SARS-CoV-2 RBD (produced in house; residues 331-550 of spike from BetaCoV/Wuhan-Hu-1/2019, accession number MN908947) at 10 μg/ml, or SARS-CoV-2 S1 (Sino Biological) at 1 μg/ml.

Wells were washed and blocked with PBS+1% BSA for 1 hour at room temperature and were then incubated with serially diluted recombinant monoclonal antibodies for 1 hour at room temperature. Plates were washed and bound antibodies were detected by incubating alkaline phosphatase-conjugated goat anti-human IgG (Southern Biotechnology: 2040-04) for 1 hour at room temperature followed by color development using 1 mg/ml p-nitrophenylphosphate substrate (Sigma-Aldrich 337 71768) in 0.1 M glycine buffer (pH 10.4) for 30 min at room temperature. The optical density (OD) values were measured at a wavelength of 405 nm in an ELISA reader (ELx808IU plate reader, BioTek).

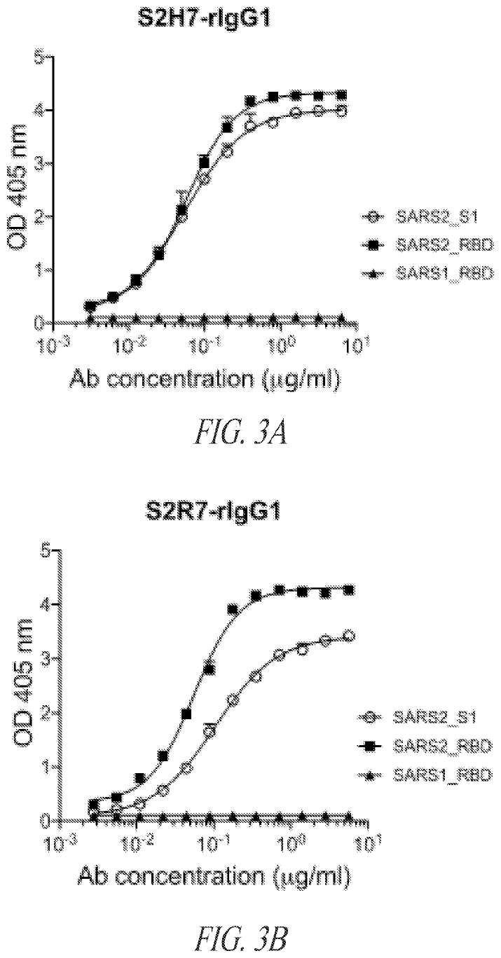

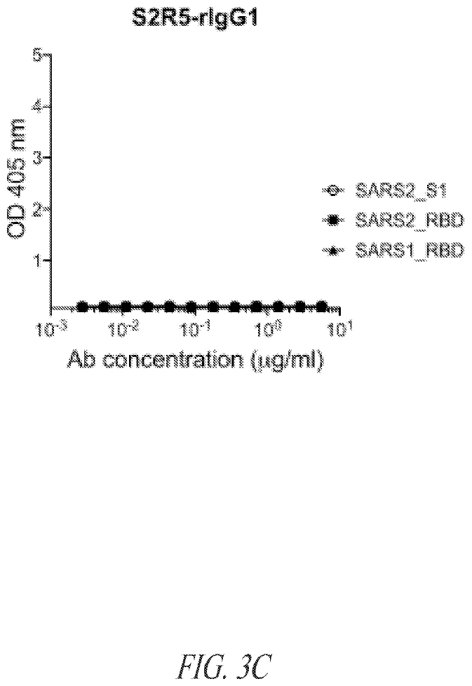

The ELISA assay results are shown in FIGS. 3A-3C, and, for antibodies S2H7 and S2R7, in Tables 7 and 8.

TABLE 7
S2H7 Binding IC50 values

| | SARS-2_S1 | SARS-2_RBD | SARS_RBD |
|---|---|---|---|
| IC50 | 0.05371 | 0.05663 | approx. 0.8615 |

TABLE 8
S2R7 Binding IC50 values

| | SARS2_S1 | SARS2_RBD | SARS1_RBD |
|---|---|---|---|
| IC50 | 0.1046 | 0.05544 | approx. 1.268 |

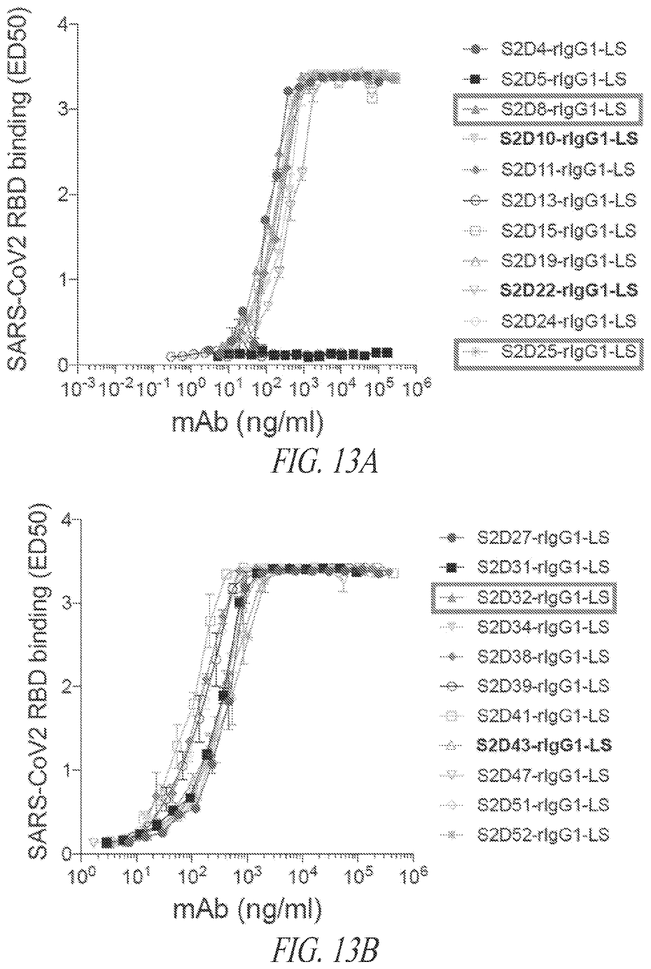

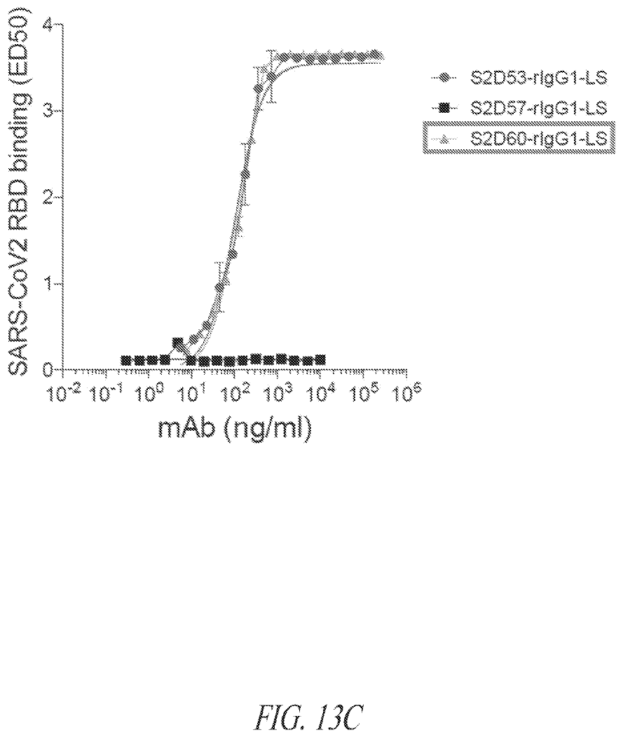

Binding of monoclonal human antibodies S2D4, S2D5, S2D8, S2D10, S2D11, S2D13, S2D15, S2D19, S2D22, S2D24, S2D25, S2D27, S2D31, S2D32, S2D34, S2D38, S2D39, S2D41, S2D43, S2D47, S2D51, S2D52, S2D53, S2D57, and S2D60, which were isolated from patients who recovered from SARS-CoV-2 infection, with the RBD of SARS-CoV-2 was determined using similar methods. Results are shown in FIGS. 13A-13C. Monoclonal antibodies S2D10, S2D22, and S2D43 also bind to RBD of SARS-CoV. Monoclonal antibodies S2D60, S2D32, S2D25, and S2D8 do not show specific binding to RBD of SARS-CoV (data not shown).

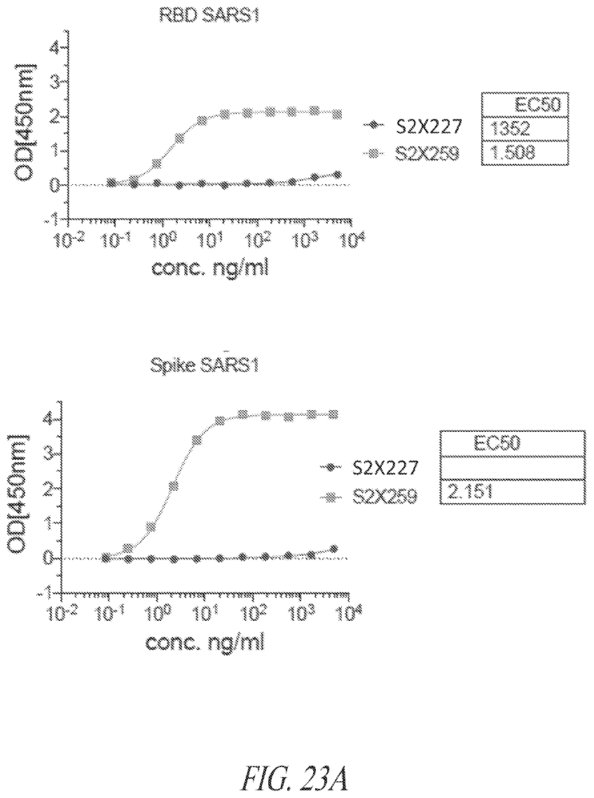

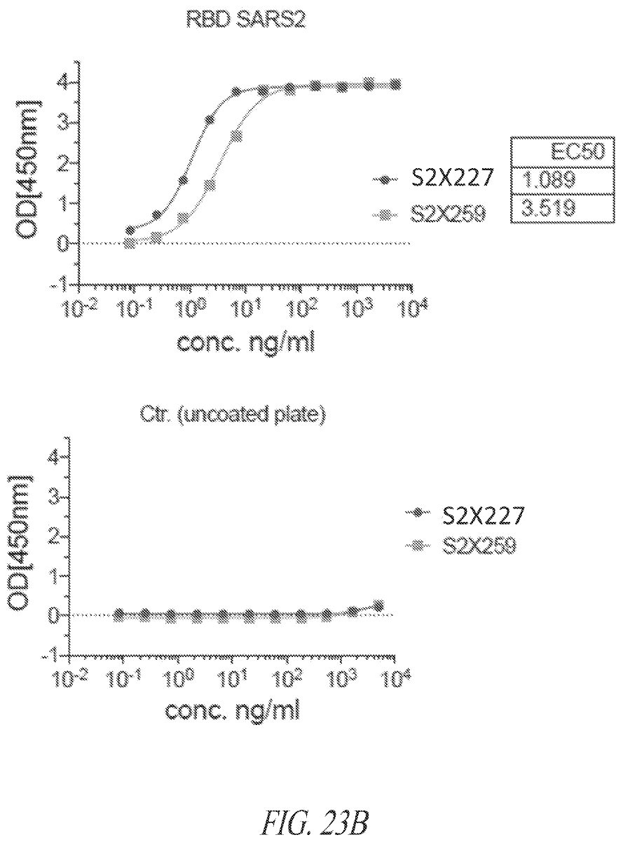

Binding of human monoclonal antibodies S2X227 and S2X259 to SARS-CoV, SARS-CoV Spike protein RBD, and SARS-CoV-2 RBD was determined using similar methods. Results are shown in FIGS. 23A and 23B.

### Example 4

### Binding of Monoclonal Antibodies to SARS-CoV-2 Spike Protein

Binding of human monoclonal antibodies S2A15-v1, S2A15-v2, S2B2-v1 and S2B2-v2, isolated from patients who recovered from SARS-CoV-2 infection, with the SARS-CoV-2 Spike protein was studied using enzyme-linked immunosorbent assay (ELISA).

96-well ELISA plates were coated with ectodomains (stabilized prefusion trimer) of SARS-CoV-2 Spike protein at 1 μg/ml.

Wells were washed and blocked with PBS+1% BSA for 1 hour at room temperature and were then incubated with serially diluted recombinant monoclonal antibodies for 1 hour at room temperature. Plates were washed and bound antibodies were detected by incubating alkaline phosphatase-conjugated goat anti-human IgG (Southern Biotechnology: 2040-04) for 1 hour at room temperature followed by color development using 1 mg/ml p-nitrophenylphosphate substrate (Sigma-Aldrich 337 71768) in 0.1 M glycine buffer (pH 10.4) for 30 min at room temperature. The optical density (OD) values were measured at a wavelength of 405 nm in an ELISA reader (ELx808IU plate reader, BioTek).

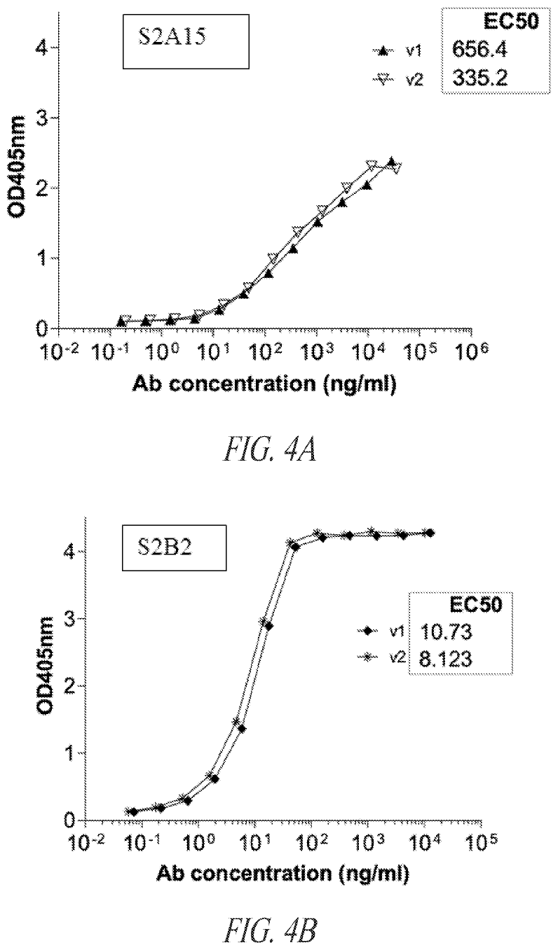

The ELISA assay results are shown in FIGS. 4A and 4B. EC50 values are given in the boxes in the upper right of each figure.

### Example 5

### Binding of Monoclonal Antibodies to RBD of SARS-CoV-2

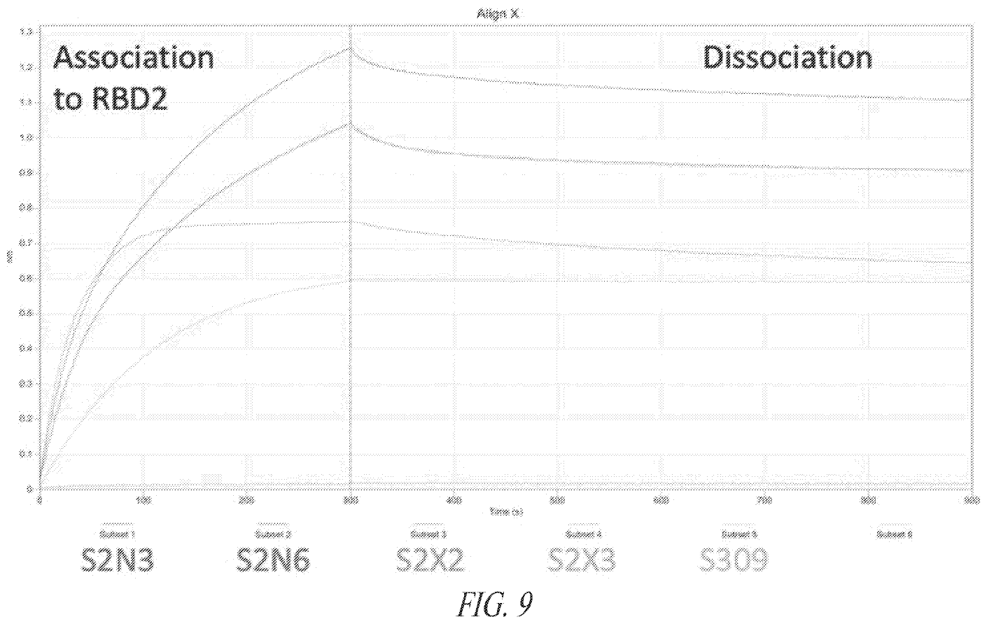

Binding of human monoclonal antibodies S2N3, S2N6, S2X2, and S2X3, isolated from patients who recovered from SARS-CoV-2 infection, and human monoclonal antibody S309, isolated from a patient who recovered from SARS-CoV infection, to the RBD of SARS-CoV-2 Spike protein was measured. Protein A sensors (Bioforte) were hydrated before loading of antibodies at 3 μg/ml in kinetics buffer for 1.5 minutes. The antibodies were from culture supernatant of transfected, monoclonal antibody-producing ExpiCHO cells. Antibody concentrations in the culture supernatant were determined by ELISA. RBD of SARS-CoV-2 (residues 331-550 of Spike protein from BetaCoV/Wuhan-Hu-1/2019, accession number MN908947) was associated at 5 g/ml for 5 minutes, then allowed to dissociate for 10 minutes. Results are shown in FIG. 9. The start of the dissociation phase is indicated in FIG. 9 with a vertical dashed line.

### Example 6

### Competitive Binding of Human Monoclonal Antibodies to RBD of SARS-CoV-2

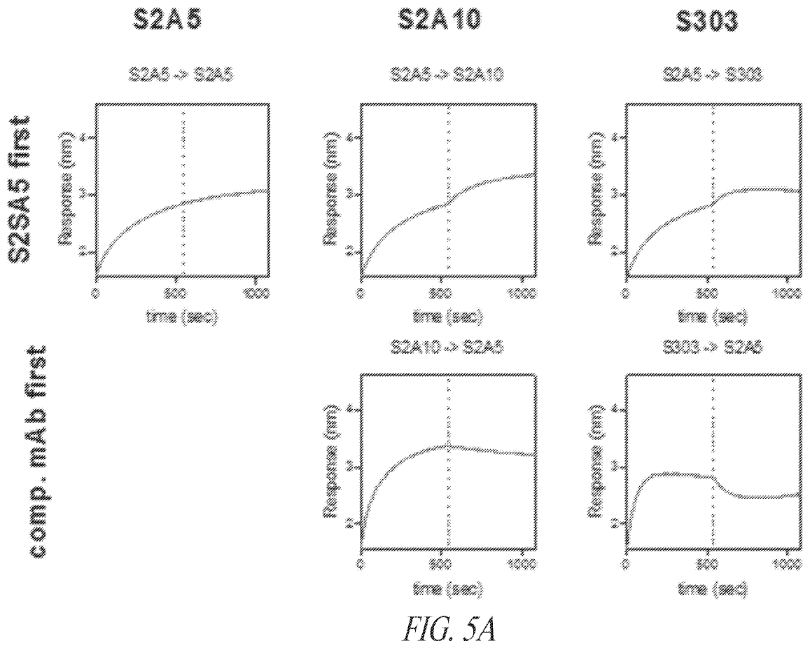

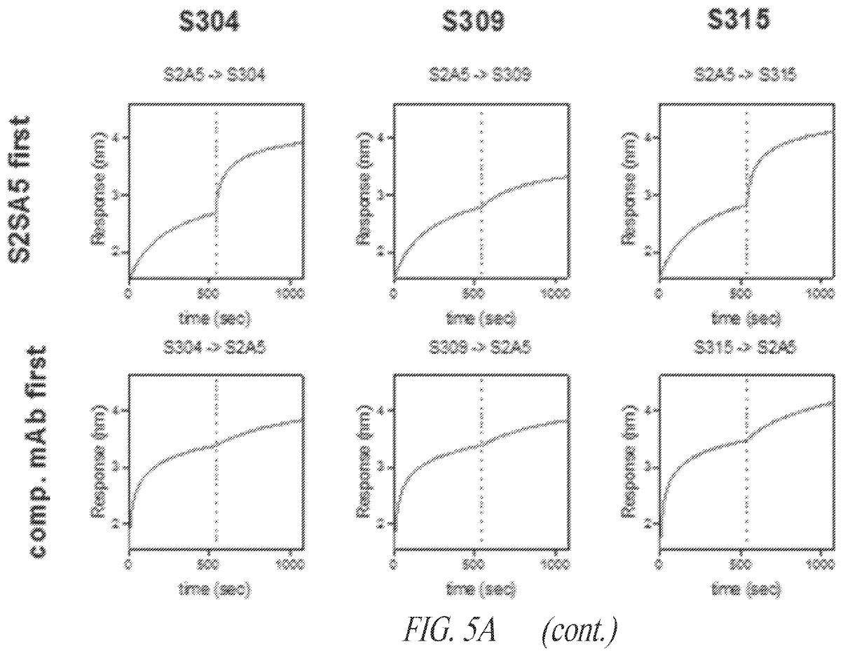

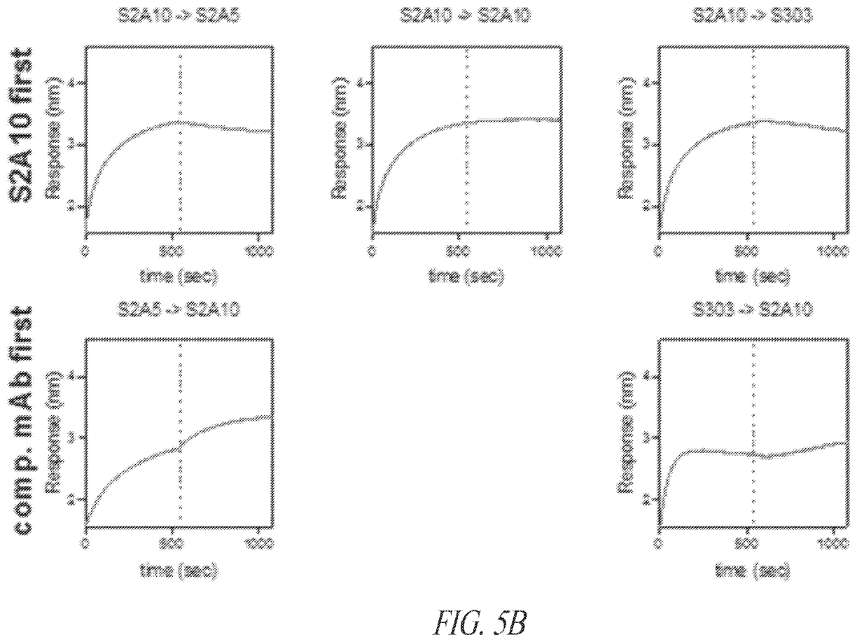

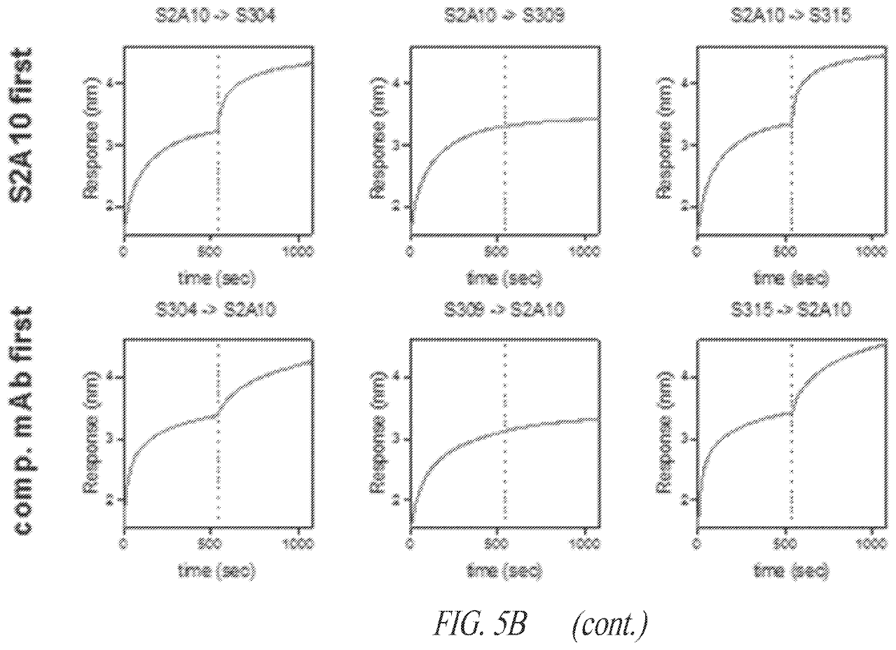

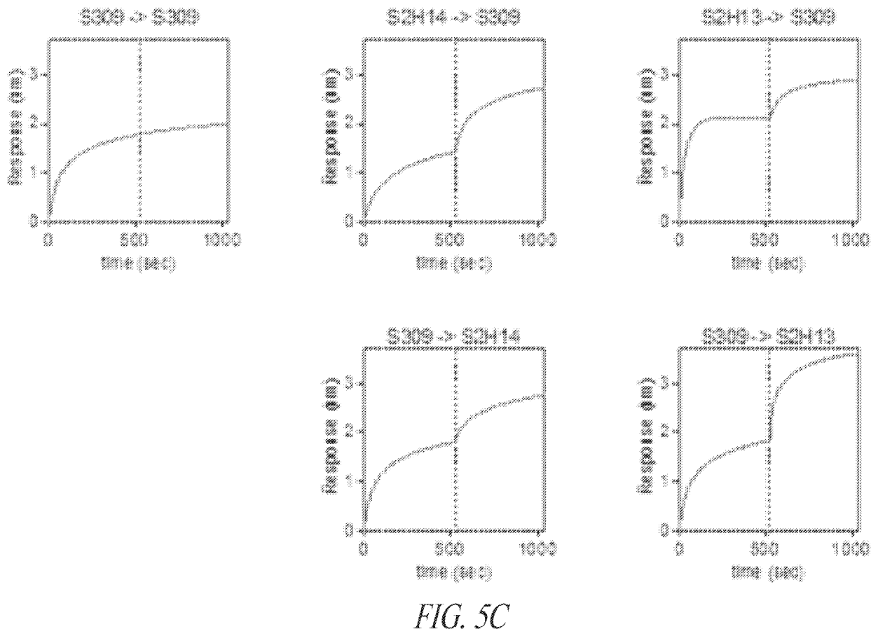

To assess overlapping binding sites of recombinant human monoclonal antibodies S2A5 and S2A10, isolated from patients who recovered from SARS-CoV-2 infection, with binding sites of recombinant human monoclonal antibodies S303, S304, S309, and S315, isolated from patients who recovered from SARS-CoV-2 infection, competition assays were performed using Octet (instrument: Octet Red96, ForteBio). Anti-His sensors (BIOSENSOR ANTI-PENTA-HIS (HIS1K)1*1^{ST}) were used to immobilize in house produced HIS-tagged RBD of SARS-CoV-2 (residues 331-550 of Spike protein from BetaCoV/Wuhan-Hu-1/2019, accession number MN908947) at a concentration of 3 μg/ml. Antibodies were associated for 6 min at 15 μg/ml. All proteins were diluted in kinetics buffer (KB). Competing antibodies were then associated at the same concentration for additional 6 mins. Results are shown in FIGS. 5A-5C.

### Example 7

### Competitive Binding of Human Monoclonal Antibodies and Human ACE2 to RBD

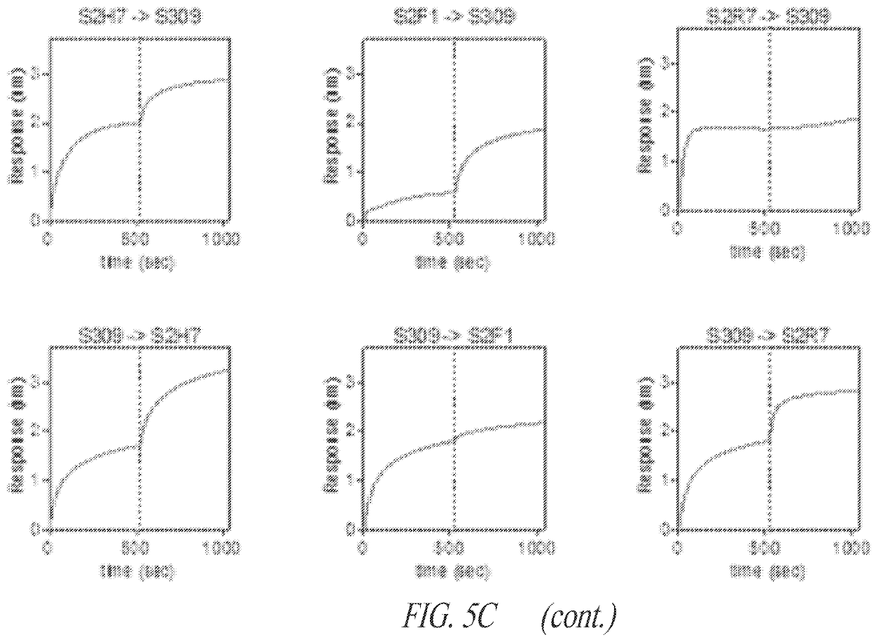

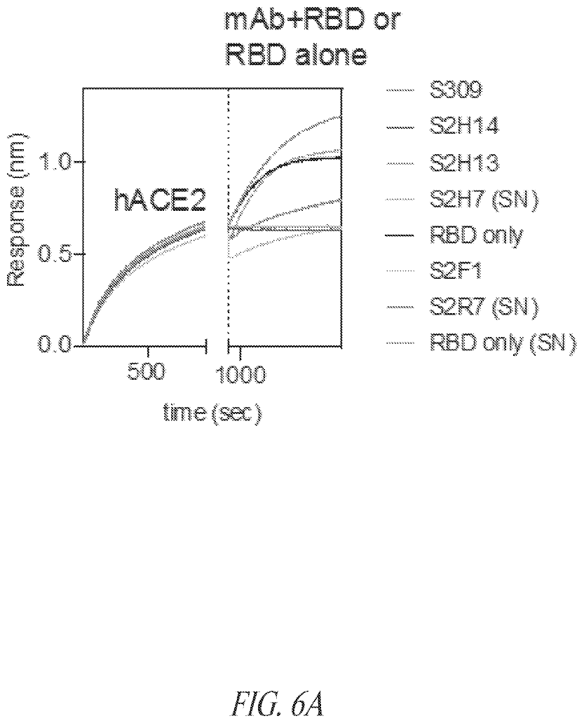

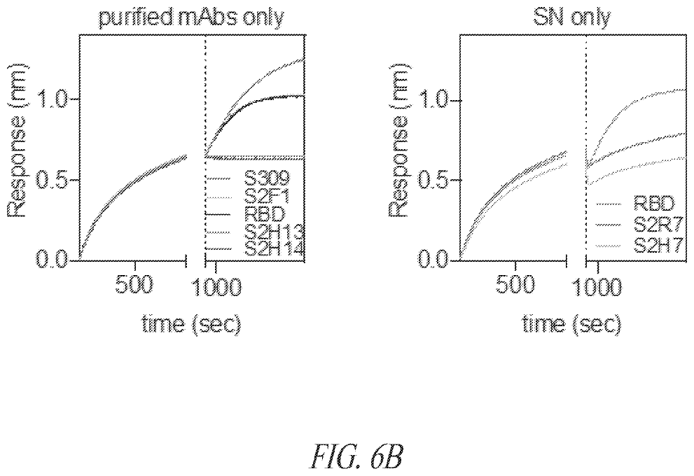

Competitive binding of recombinant human monoclonal antibodies and human ACE2 to RBD was measured. Human ACE2-His (Bio-Techne AG) was loaded for 30 minutes at 5 μg/ml in kinetics buffer (KB) onto anti-HIS (HIS1K) biosensors (Molecular Devices-ForteBio). 1 μg/ml SARS-CoV-2 RBD-mouse Fc (Sino Biological Europe GmbH) at was preincubated with or without antibody (30 μg/ml for 30 minutes) before measurement of RBD association to hACE2 for 15 minutes. FIG. 6A shows competitive binding of monoclonal antibodies S309, S2H14, S2H13, S2H7, S2F1, and S2R7 with ACE2 to RBD. FIG. 6B shows competitive binding of antibodies with ACE2 to RBD; antibodies were tested as purified recombinant antibody (left panel) and as ExpiCHO culture supernatant (SN) (right panel). The vertical dashed line in each of FIGS. 6A and 6B indicates the start of the loading of RBD with or without antibody.

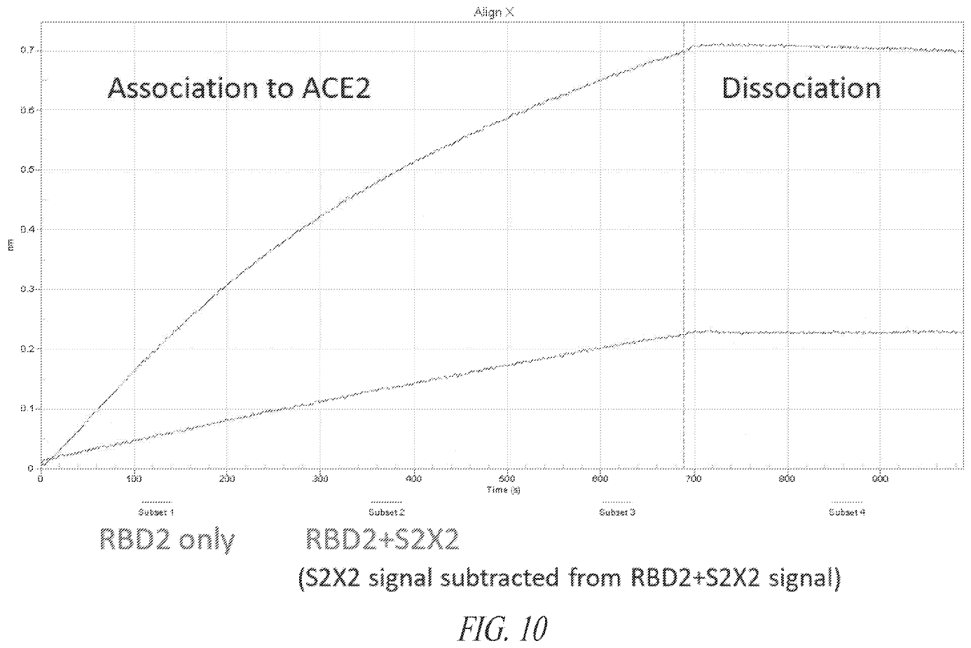

Further studies were carried out to measure competitive binding of recombinant human antibody S2X2 and human ACE2 to RBD. Human ACE2-His (Sino Biological) was loaded for 30 minutes at 2 μg/ml in kinetics buffer onto anti-HIS (HIS1K) biosensors (Molecular Devices—ForteBio). SARS-CoV-2 RBD (produced in house; residues 331-550 of spike protein from BetaCoV/Wuhan-Hu-1/2019, accession number MN908947) at 0.5 μg/ml was pre-incubated with or without mAb S2X2 (15 μg/ml, 30 minutes) before measurement of RBD association to hACE2 for 10 minutes. Dissociation was recorded for 5 minutes. Antibodies were in the form of cell culture supernatant from transfected ExpiCHO cells. Results are shown in FIG. 10. The start of the dissociation phase is indicated in FIG. 10 with a vertical dashed line.

### Example 8

### Neutralization of SARS-CoV-2 by Recombinant Human Monoclonal Antibodies

Human monoclonal antibodies isolated from patients who recovered from SARS-CoV-2 infection were expressed recombinantly and were tested in neutralization assays against SARS-CoV-2 pseudotyped virus (VSV).

Recombinant monoclonal antibodies were serially diluted and incubated with a constant amount of VSV-deltaG-luc pseudotyped with SARS-CoV-2 (strain BetaCoV/Wuhan-Hu-1/2019, accession number MN908947) for 1.5 hours at 37° C. VeroE6 cells were then added in complete DMEM medium and plates were incubated for 24 hours at 37° C. To measure the amount of luciferase expressed in infected cells, culture medium was aspirated and luciferase substrate Bio-Glo Luciferase assay system (Promega AG) warmed to room temperature was added. After 10 minutes incubation in the dark on a shaker, signals were measured in a luminometer using 1 second integration time.

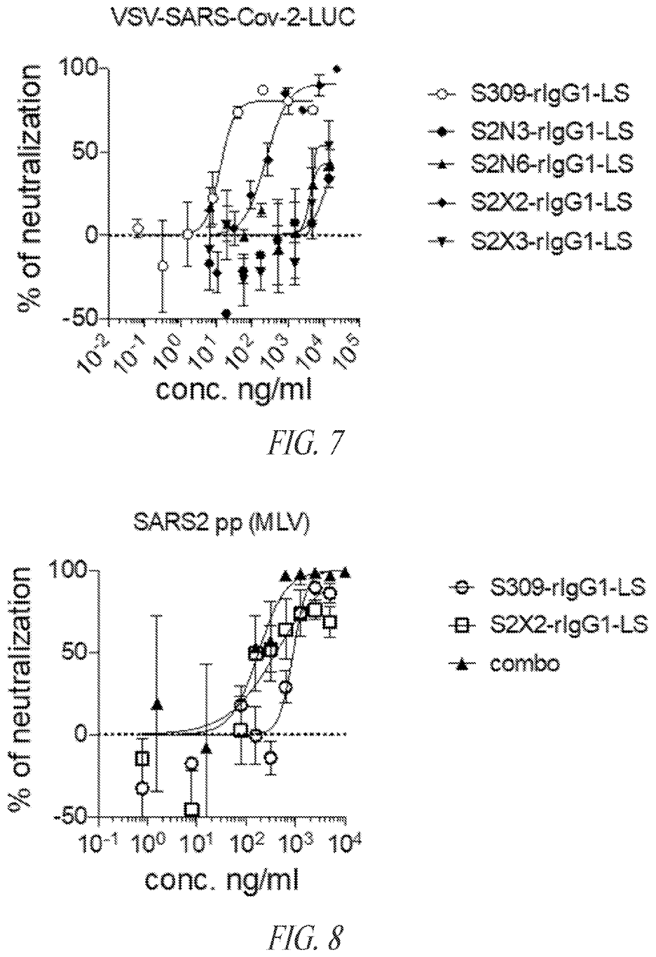

Results for recombinant monoclonal antibodies S2X2, S2X2, S2N3, and S2N6, along with results for recombinant monoclonal antibody S309, which was isolated from a patient who recovered from SARS-CoV infection, are shown in FIG. 7. IC50 values (ng/ml) are shown in Table 9. All antibodies were tested as LS Fc variants.

TABLE 9

| | S309- | S2N3- | S2N6- | S2X2- | S2X3- |
|---|---|---|---|---|---|
| | rIgG1-LS | rIgG1-LS | rIgG1-LS | rIgG1-LS | rIgG1-LS |
| IC50 | 12.75 | 21200 | 4001 | 247.2 | approx. 4882 |

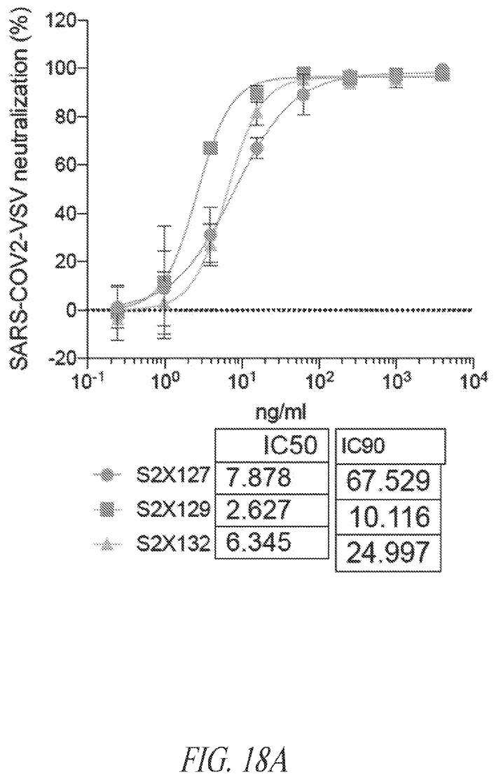

Neutralization of infection by antibodies S2X127, S2X129, S2X132, and S2X190 was assayed using similar methods. Results are shown in FIGS. 18A and 18B. Calculated IC50 and IC90 values are shown below each graph.

### Example 9

### Neutralization of SARS-CoV-2 by Recombinant Human Monoclonal Antibodies

Human monoclonal antibodies isolated from patients who recovered from SARS-CoV-2 infection were expressed recombinantly and were tested in neutralization assays against live SARS-CoV-2 virus.

Vero E6 cells cultured in DMEM supplemented with 10% FBS (VWR) and 1× Penicillin/Streptomycin (Thermo Fisher Scientific) were seeded in white 96-well plates at 20,000 cells/well and attached overnight. Serial 1:4 dilutions of the monoclonal antibodies were incubated with 200 pfu of SARS-CoV-2 (isolate USA-WA1/2020, passage 3, passaged in Vero E6 cells) for 30 minutes at 37° C. in a BSL-3 facility. Cell supernatant was removed and the virus-antibody mixture was added to the cells. 24 hours post infection, cells were fixed with 4% paraformaldehyde for 30 minutes, followed by two PBS (pH 7.4) washes and permeabilization with 0.25% Triton X-100 in PBS for 30 minutes. After blocking in 5% milk powder/PBS for 30 minutes, cells were incubated with a primary antibody targeting SARS-CoV-2 nucleocapsid protein (Sino Biological, cat. 40143-R001) at a 1:2000 dilution for 1 hour. After washing and incubation with a secondary Alexa647-labeled antibody mixed with 1 μg/ml Hoechst33342 for 1 hour, plates were imaged on an automated cell-imaging reader (Cytation 5, Biotek) and nucleocapsid-positive cells were counted using the manufacturer's supplied software. Data were processed using Prism software (GraphPad Prism 8.0).

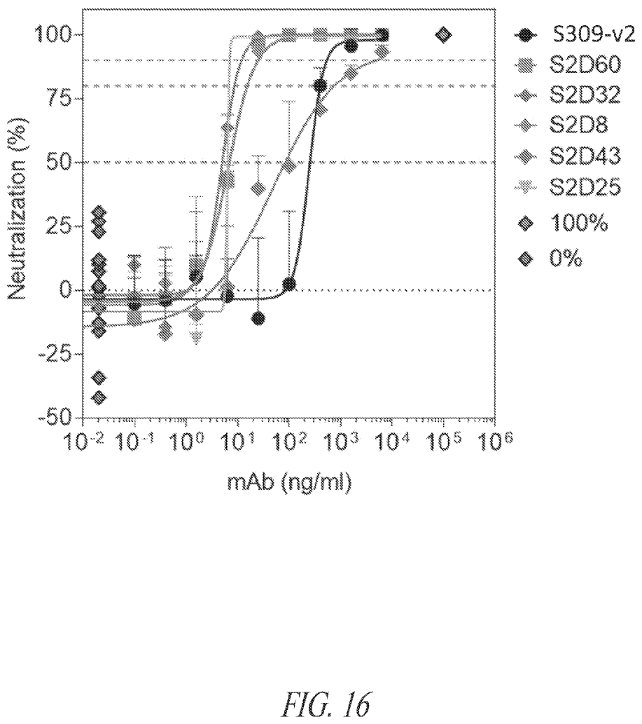

Results are shown in FIG. 16 and Table 10. Calculated IC50 values are shown in Table 11 (ng/ml). Calculated IC50 and IC90 values are shown in Table 12 (ng/ml). Comparator antibody “S309-v2” in FIG. 16 comprises the VH amino acid sequence set forth in SEQ ID NO.:340 and the VL amino acid sequence set forth in SEQ ID NO.:344, and is an engineered variant of an antibody isolated from a patient who recovered from SARS-CoV infection.

TABLE 10

| X | S309-v2 | S2D60 | S2D32 | S2D8 | S2D43 | S2D25 |
|---|---|---|---|---|---|---|
| (interpolated) | (Entered) | (Entered) | (Entered) | (Entered) | (Entered) | (Entered) |
| 538.271 | 90.000 | | | | | |
| 253.345 | 50.000 | | | | | |
| 25.014 | | 90.000 | | | | |
| 6.835 | | 50.000 | | | | |
| 11.847 | | | 90.000 | | | |
| 5.029 | | | 50.000 | | | |
| 24.765 | | | | 90.000 | | |
| 6.842 | | | | 50.000 | | |
| 4918.950 | | | | | 90.000 | |
| 83.335 | | | | | 50.000 | |
| 6.971 | | | | | | 90.000 |
| 6.312 | | | | | | 50.000 |

TABLE 11

| Antibody | IC50 (ng/mL) |
|---|---|
| S309-v2 | 244.6 |
| S2D60 | 6.395 |
| S2D32 | 4.956 |
| S2D8 | 6.494 |
| S2D43 | 48.19 |
| S2D25 | approx. 6.263 |

TABLE 12

| | IC50 | IC90 |
|---|---|---|
| Antibody | (ng/mL) | (ng/mL) |
| S2D60 | 7 | 25 |
| S2D8 | 7 | 25 |
| S2D25 | 6 | 7 |
| S2D32 | 5 | 12 |

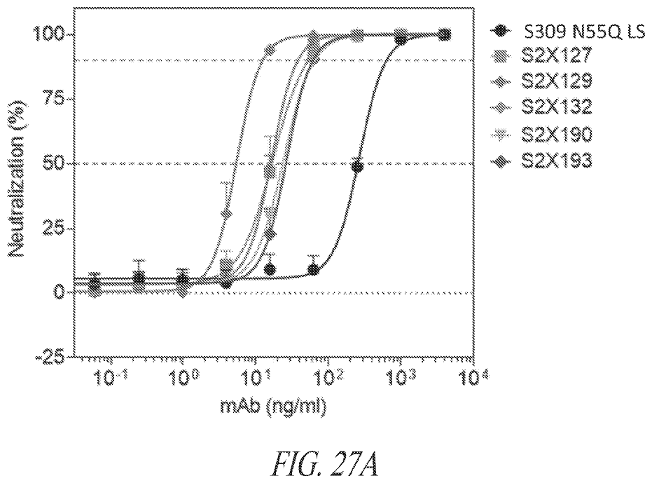

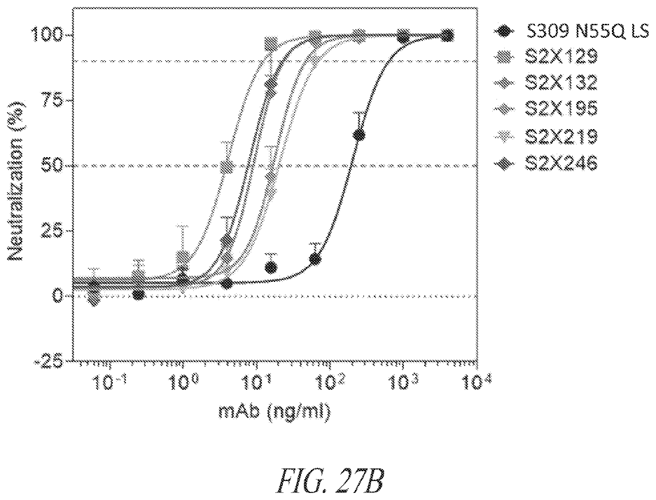

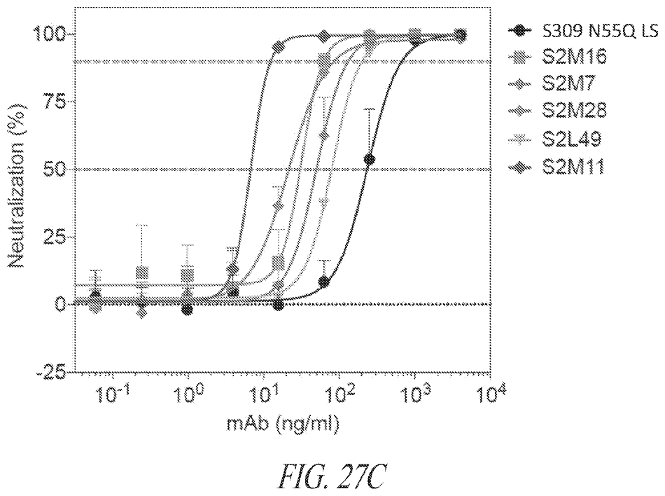

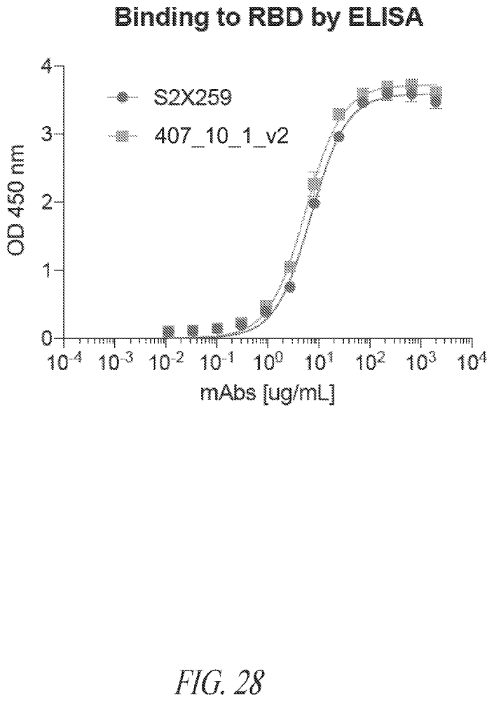

Neutralization assays were performed for additional monoclonal antibodies using similar methods. Results are shown in FIGS. 27A-27C, 29, and 30. FIG. 27A shows results for antibody S2X193, along with five comparator antibodies, including S309 N55Q LS. S309 N55Q LS comprises the VH amino acid sequence set forth in SEQ ID NO:340 and the VL amino acid sequence set forth in SEQ ID NO: 344, and comprises an MLNS modification in the Fc region. IC50 values for the tested antibodies were measured to be 268.4 ng/ml for S309 N55Q LS, 17.18 ng/ml for S2X127, 5.379 ng/ml for S2X129, 16.50 ng/ml for S2X132, 24.18 ng/ml for S2X190, and 26.69 ng/ml for S2X193. Interpolated EC50 and EC90 values for the tested antibodies are shown in Table 13 (ng/ml).

TABLE 13

| Antibody | EC50 | EC90 |
|---|---|---|
| S309 N55Q LS | 256 | 635 |
| S2X127 | 17 | 54 |
| S2X129 | 5 | 12 |
| S2X132 | 16 | 38 |
| S2X190 | 23 | 64 |
| S2X193 | 26 | 61 |

FIG. 27B shows results for exemplary antibodies S2X195, S2X219, and S2X246, along with three comparator antibodies. IC50 values for the tested antibodies were measured to be 205.4 ng/ml for S309 N55Q LS, 4.104 ng/ml for S2X129, 9.265 ng/ml for S2X132, 17.85 ng/ml for S2X195, 20.63 ng/ml for S2X219, and 7.894 ng/ml for S2X246. Interpolated EC50 and EC90 values for the tested antibodies are shown in Table 14 (ng/ml).

TABLE 14

| Antibody | EC50 | EC90 |
|---|---|---|
| S309 N55Q LS | 194 | 634 |
| S2X129 | 4 | 12 |
| S2X132 | 9 | 24 |
| S2X195 | 17 | 47 |
| S2X219 | 20 | 69 |
| S2X246 | 8 | 22 |

FIG. 27C shows results for five antibodies and comparator antibody S309 N55Q LS. IC50 values for the tested antibodies were measured to be 236.4 ng/ml for S309 N55Q LS, 31.85 ng/ml for S2M16, 50.24 ng/ml for S2M7, 21.22 ng/ml for S2M28, 80.37 ng/ml for S2L49, and 6.774 ng/ml for S2M11. Interpolated EC50 and EC90 values for the tested antibodies are shown in Table 15 (ng/ml).

TABLE 15

| Antibody | EC50 | EC90 |
|---|---|---|
| S309 N55Q LS | 233 | 670 |
| S2M16 | 30 | 62 |
| S2M7 | 50 | 131 |
| S2M28 | 21 | 85 |
| S2L49 | 79 | 200 |
| S2M11 | 7 | 12 |

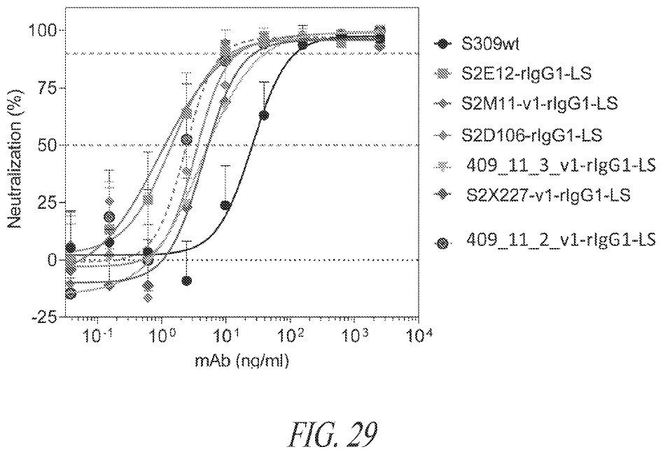

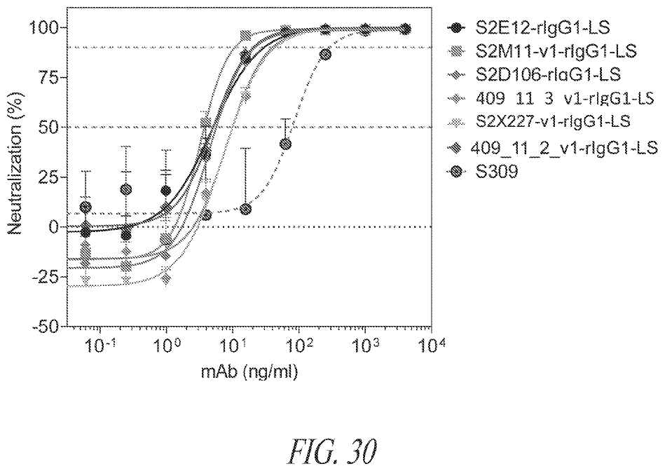

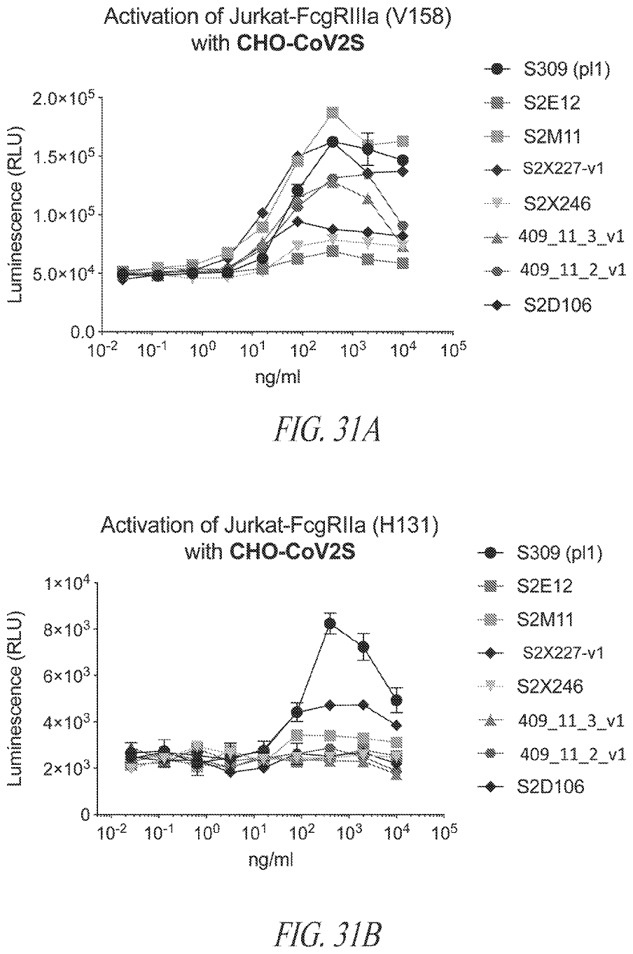

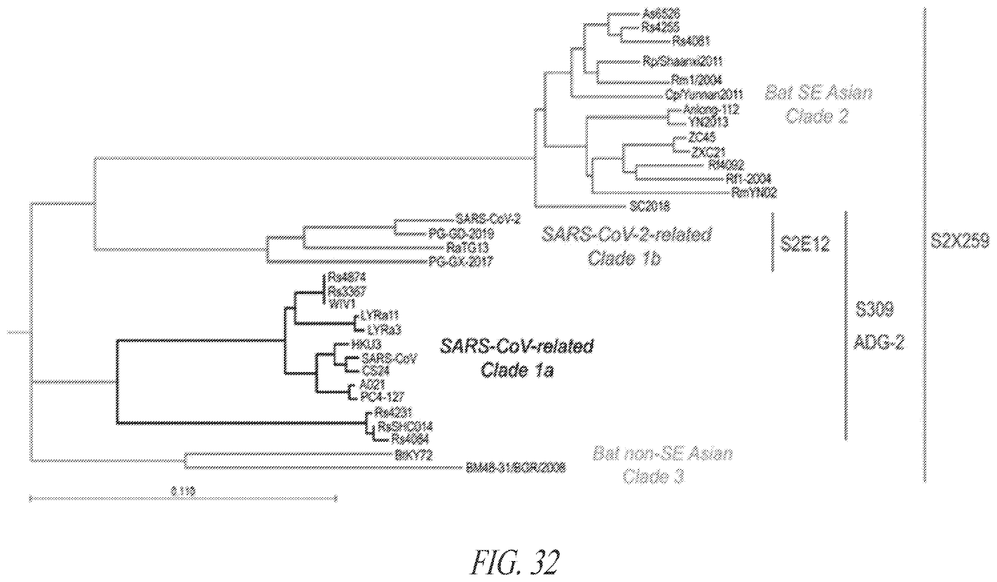

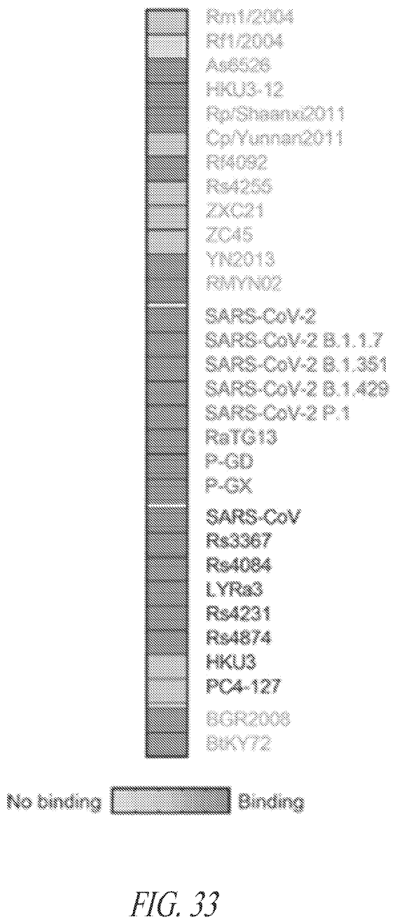

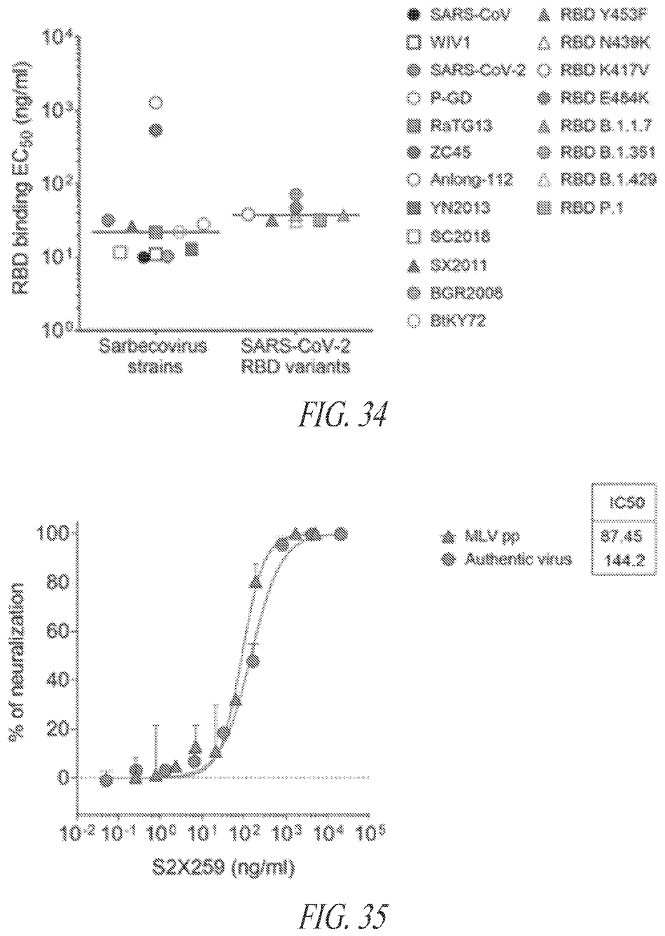

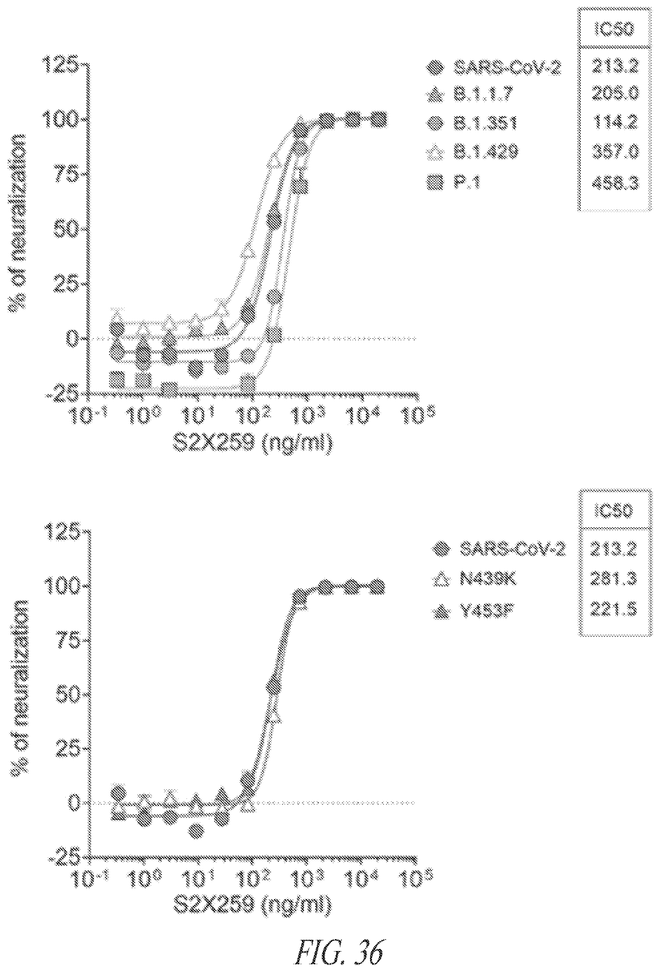

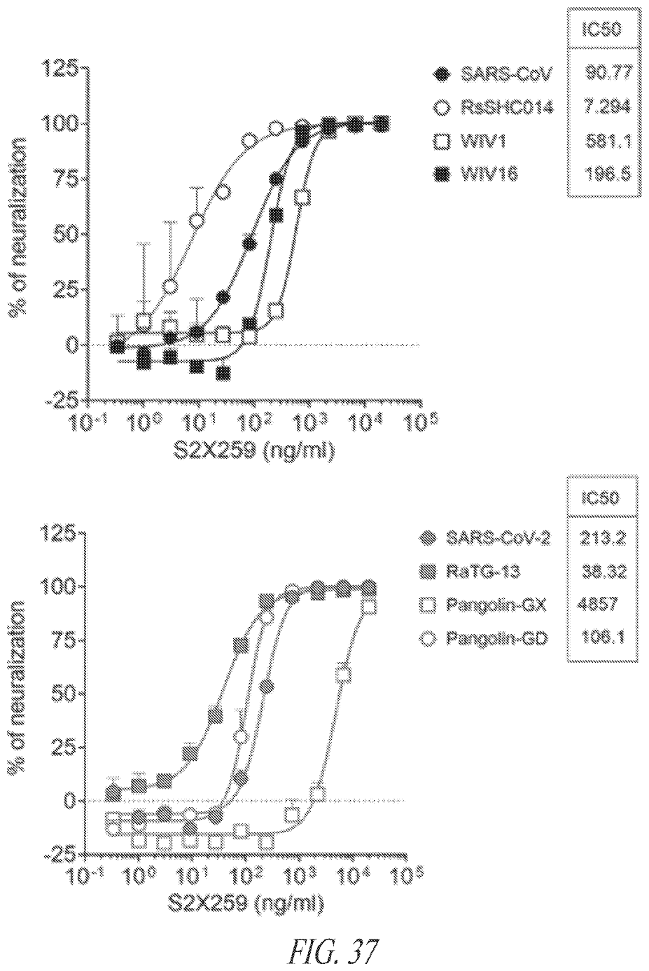

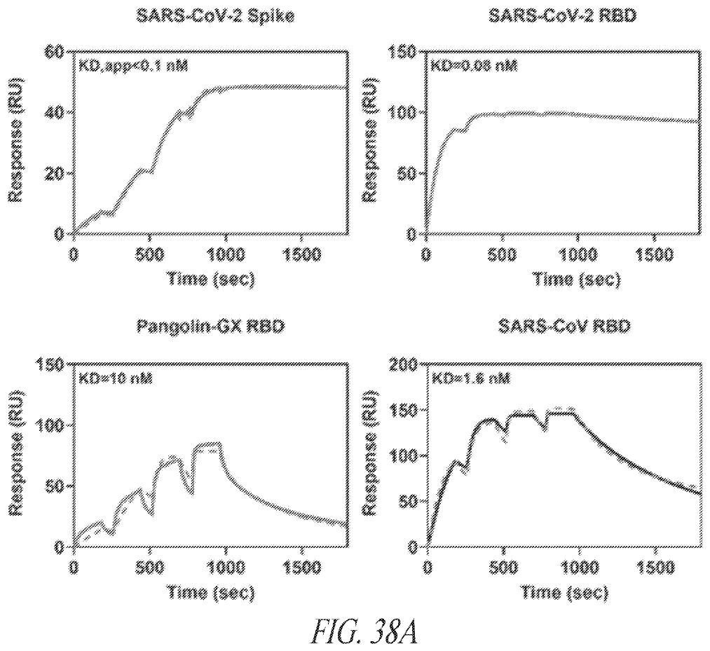

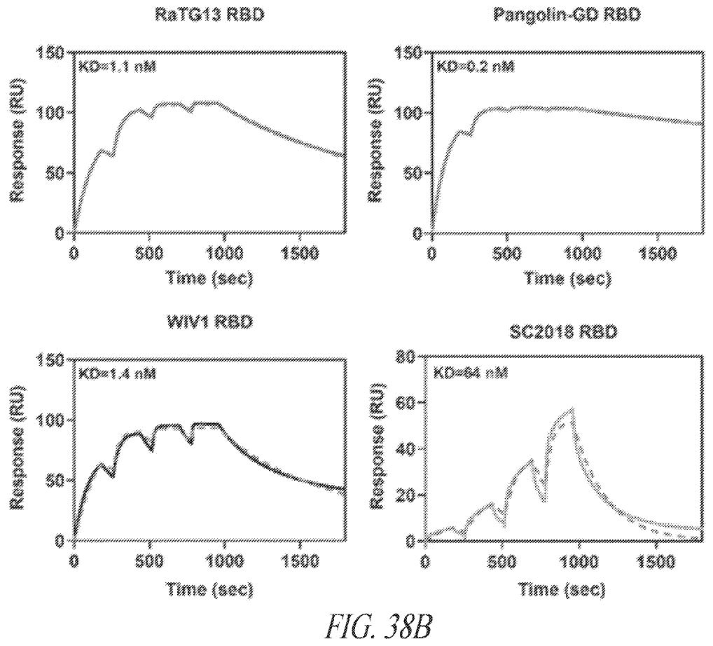

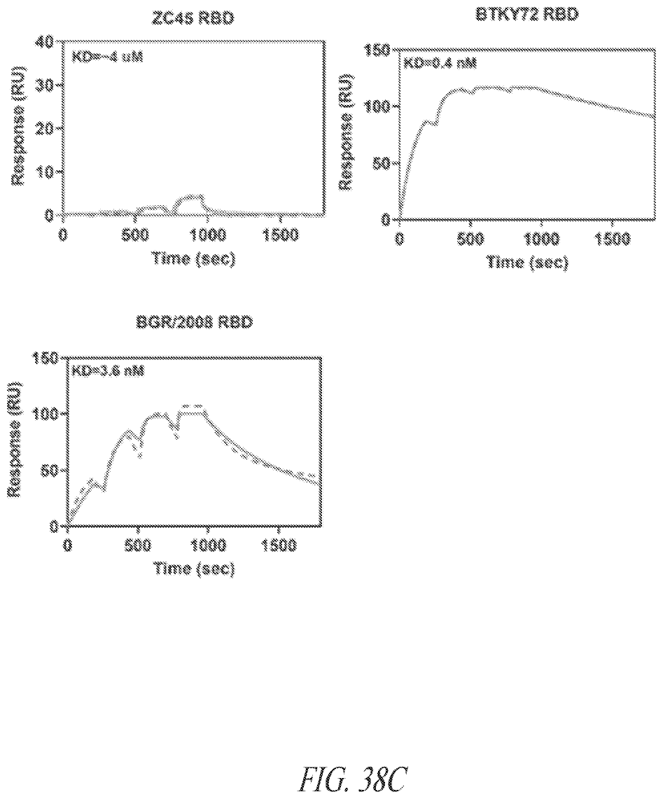

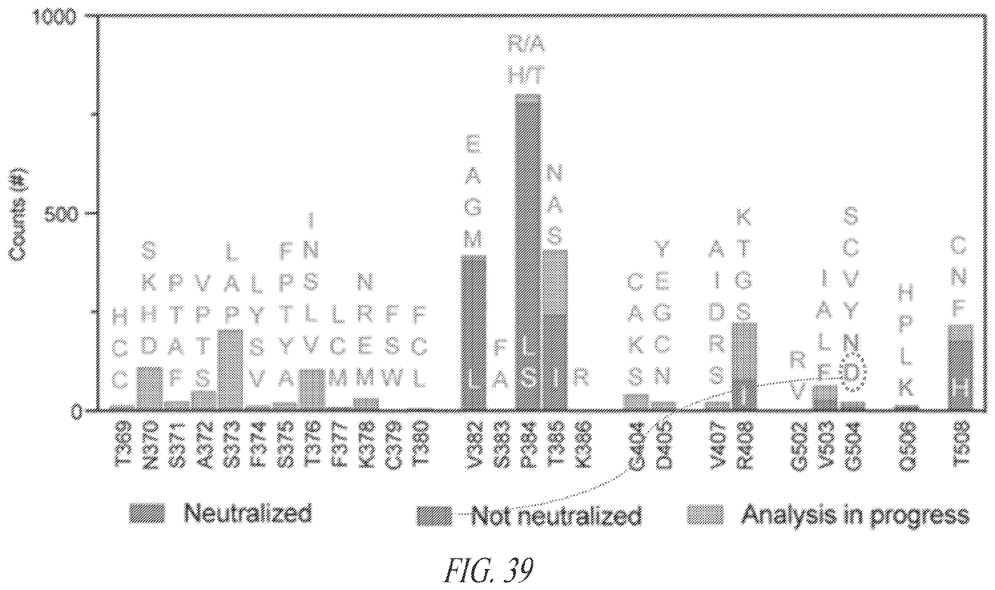

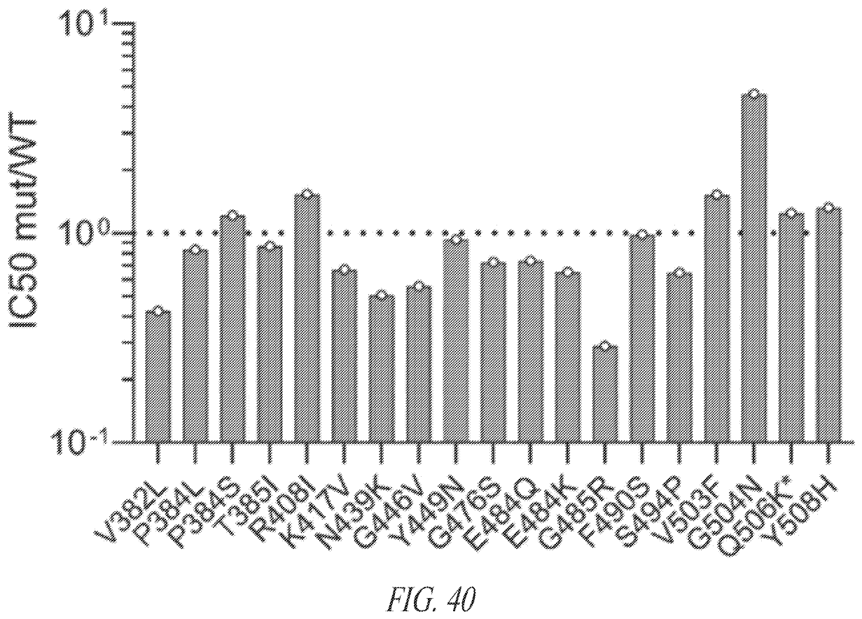

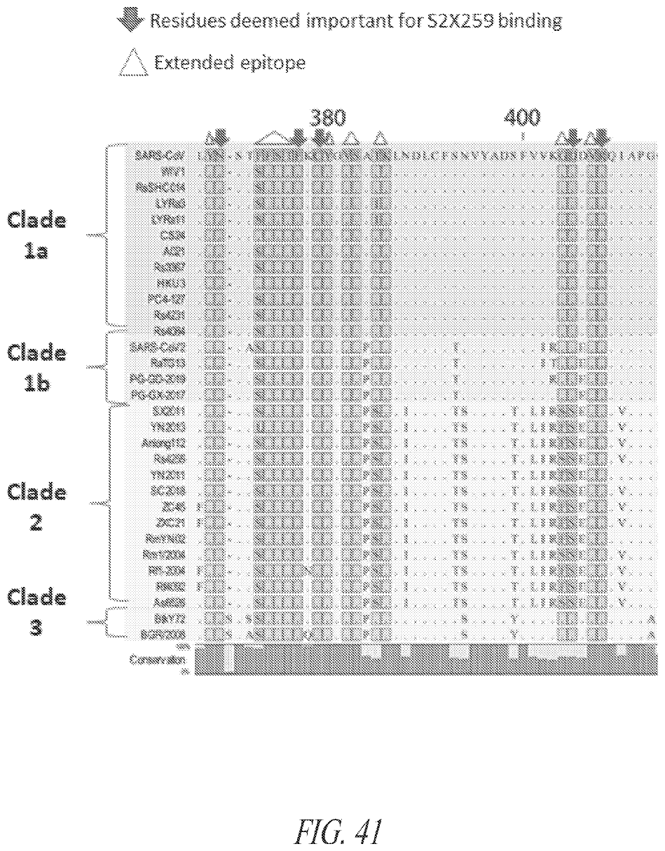

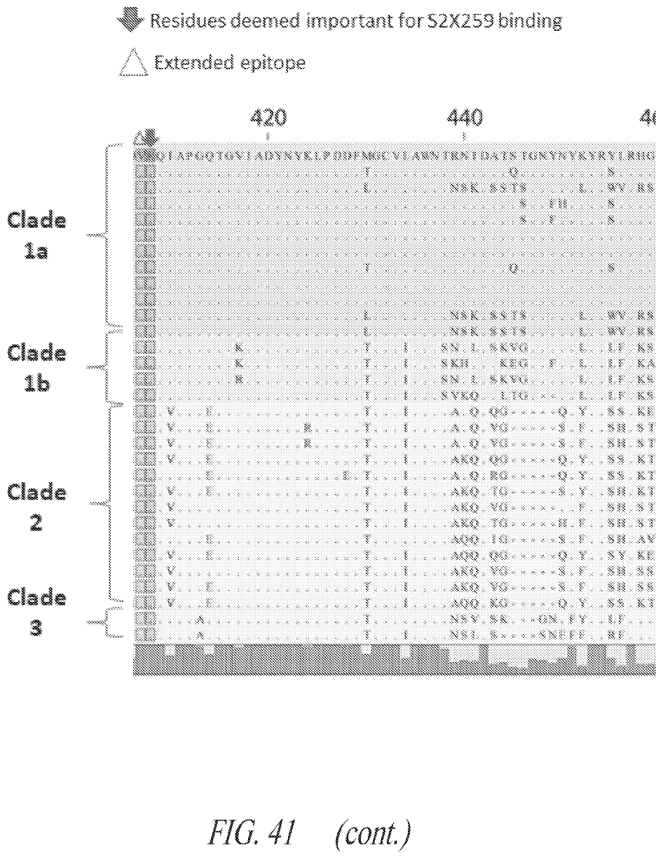

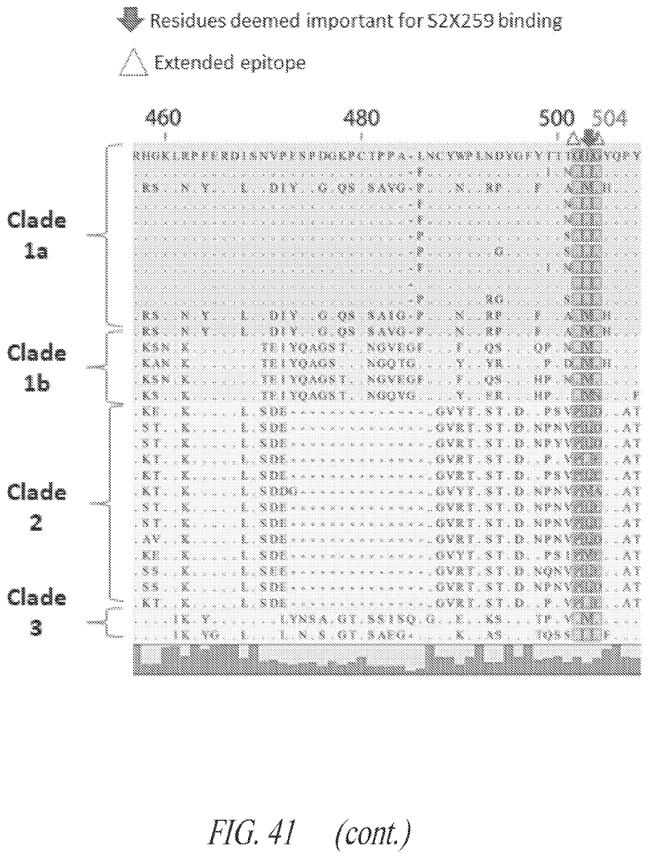

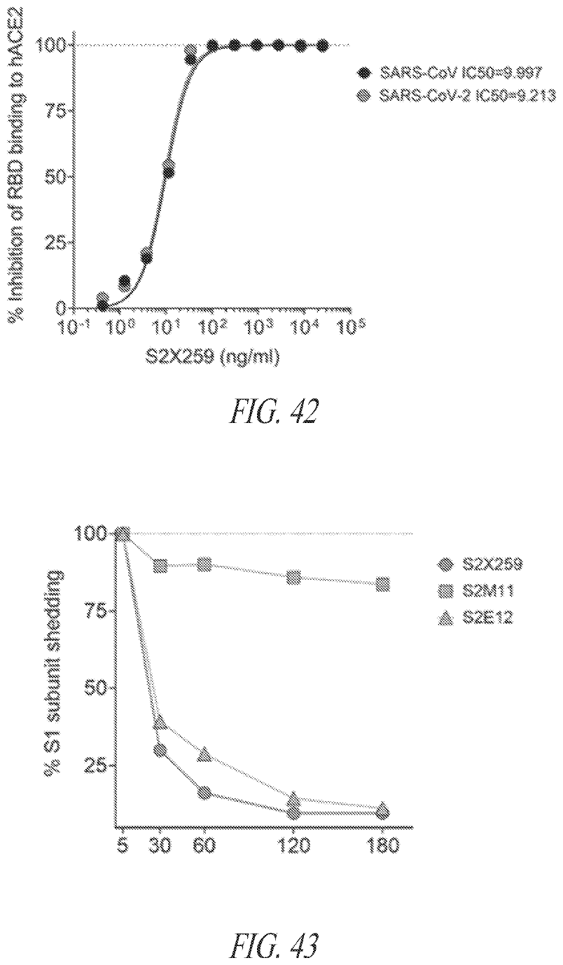

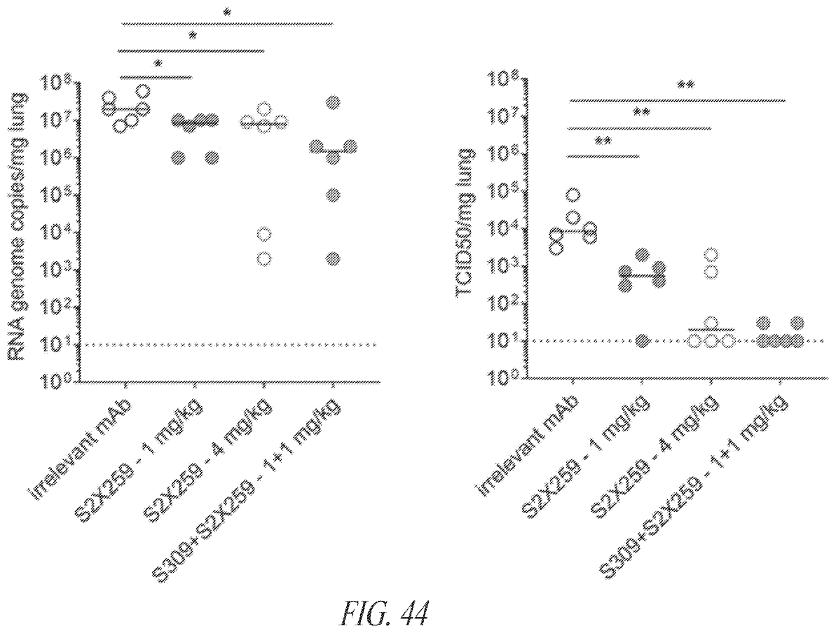

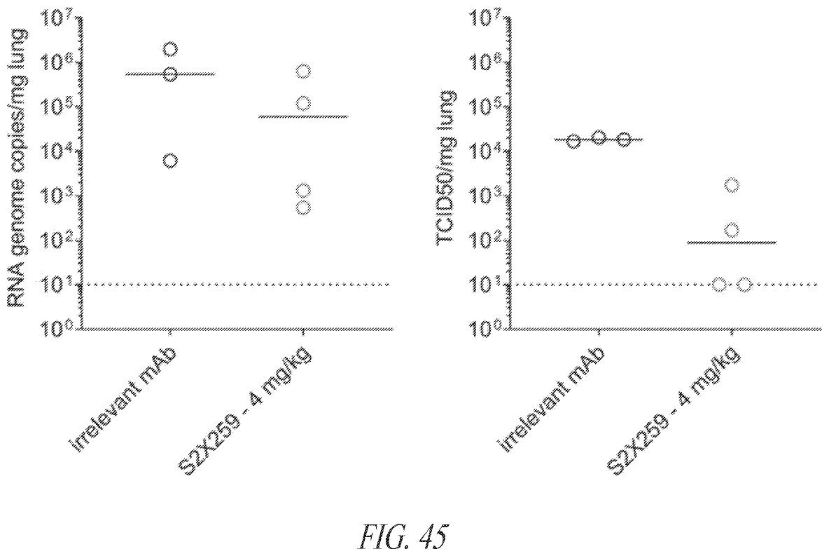

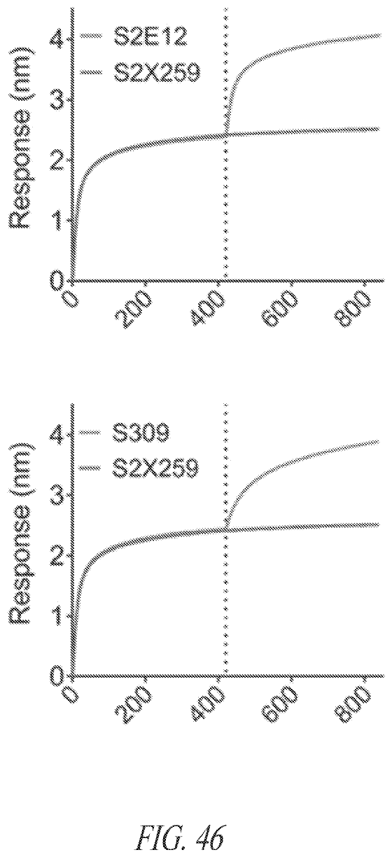

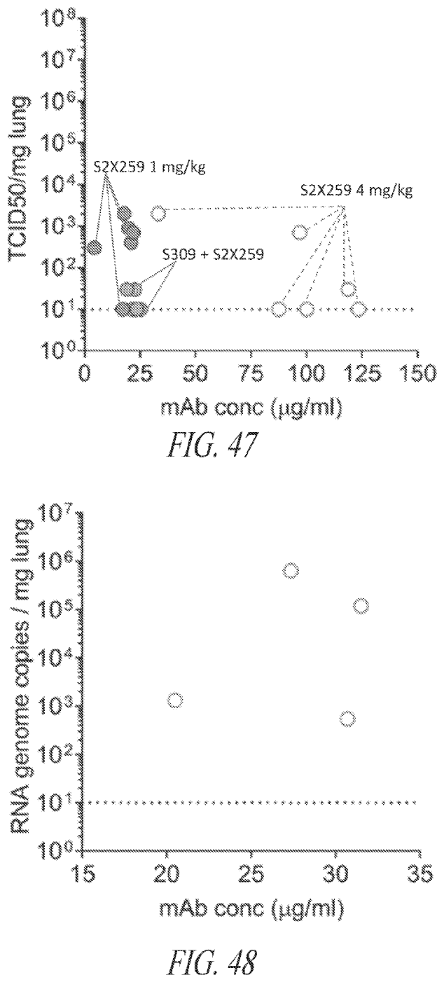

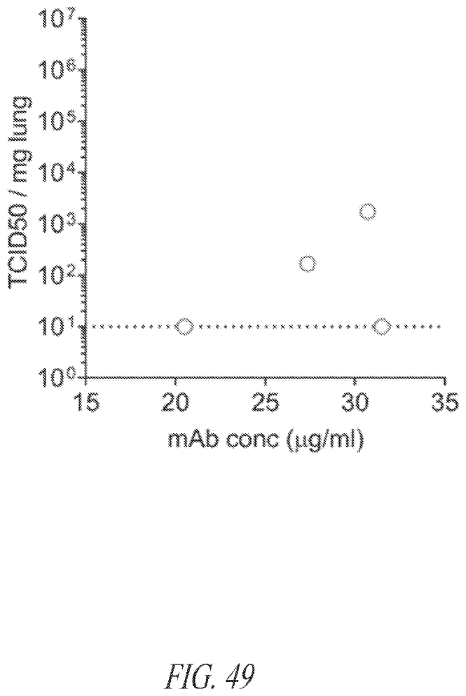

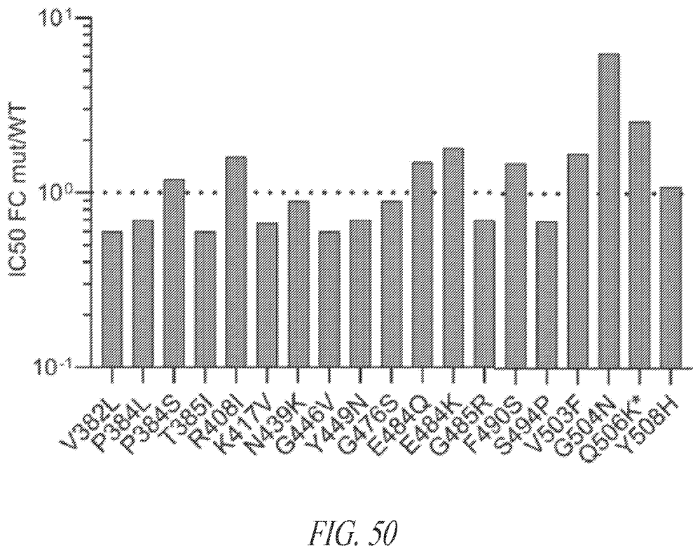

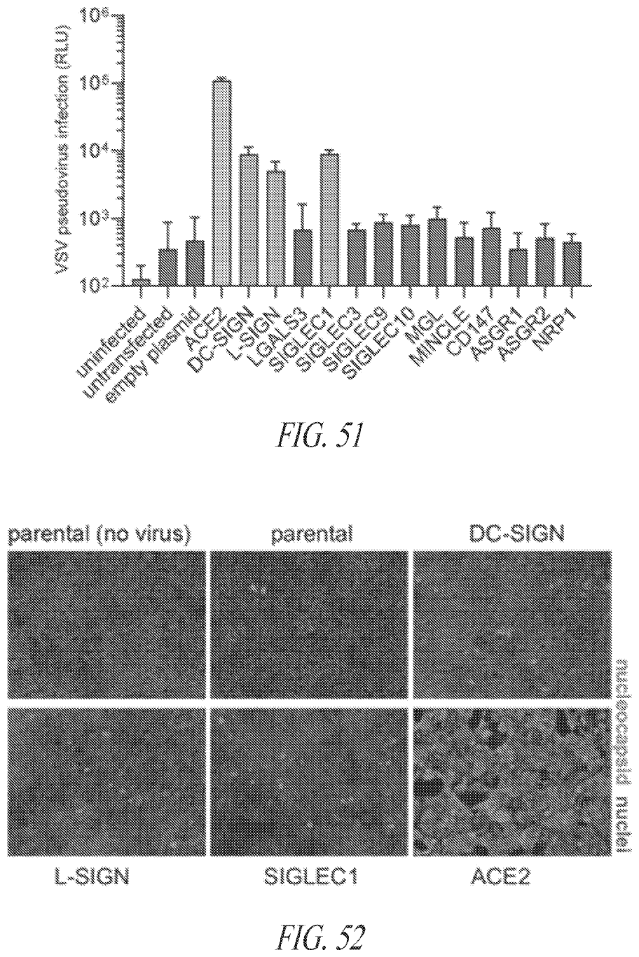

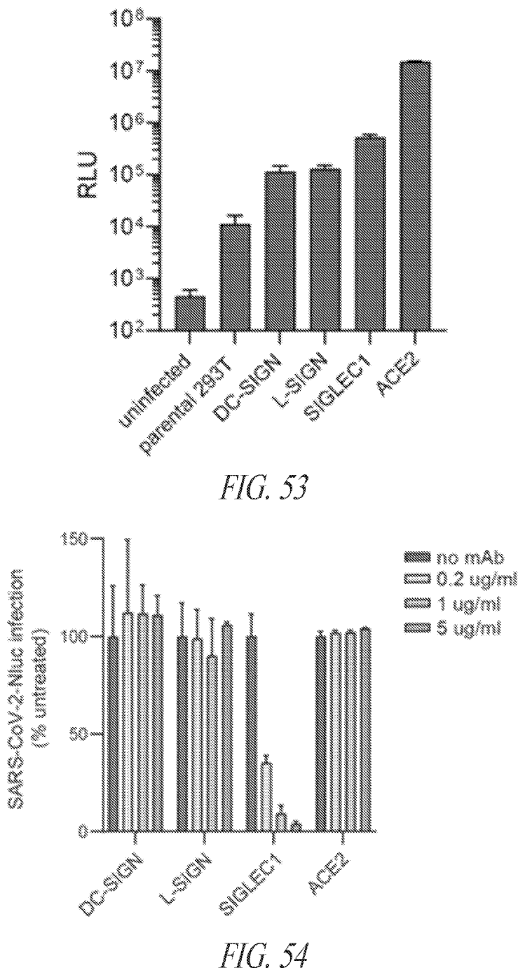

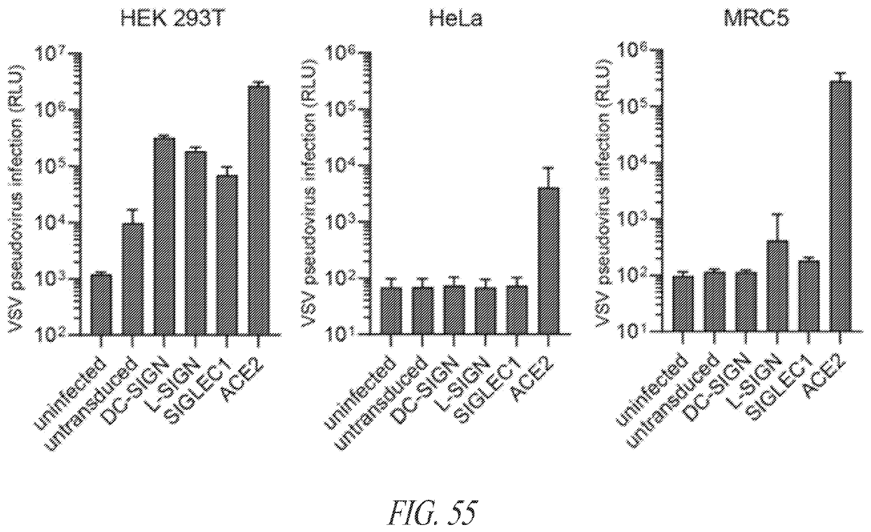

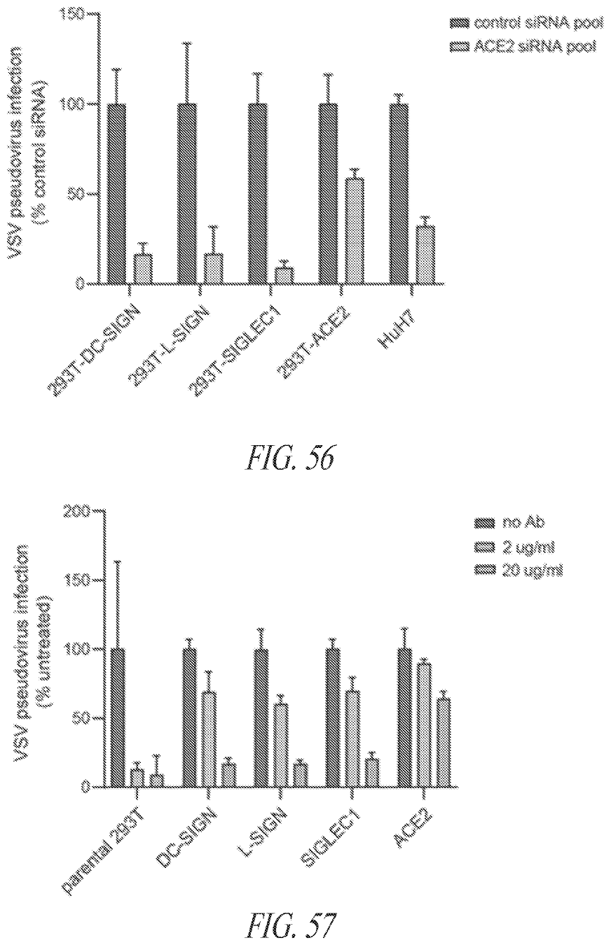

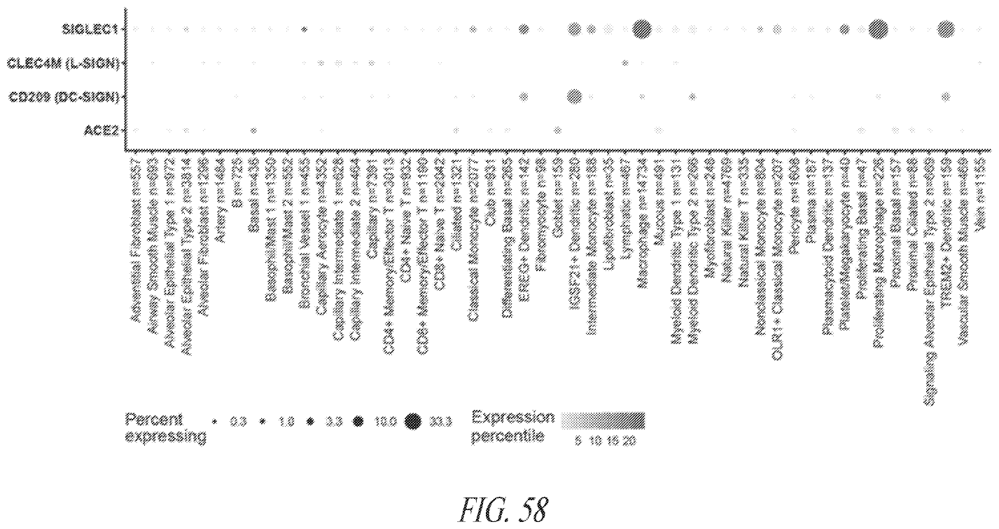

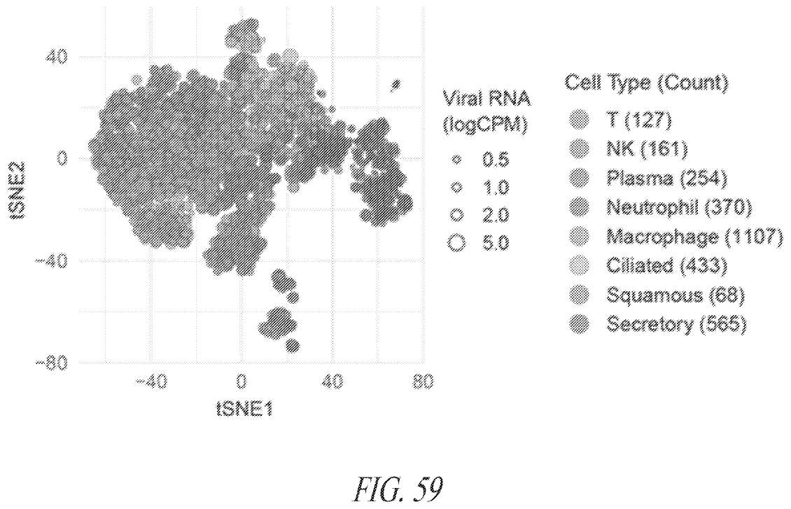

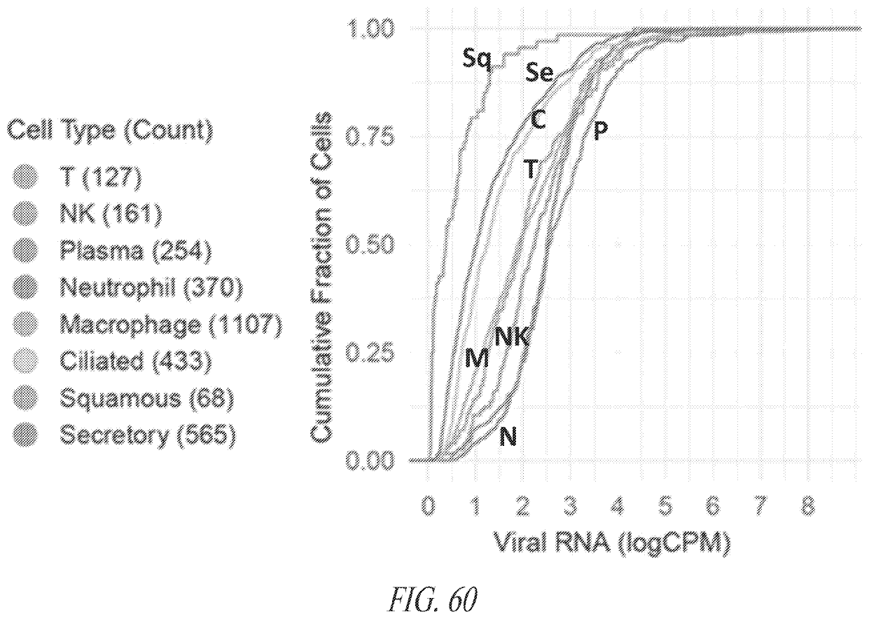

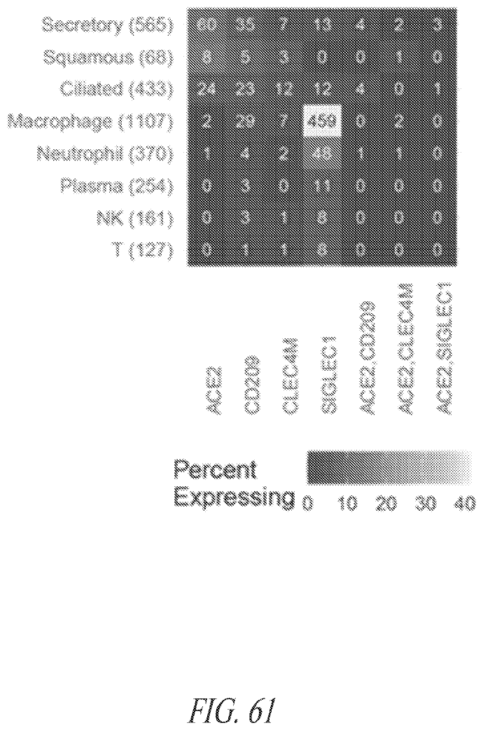

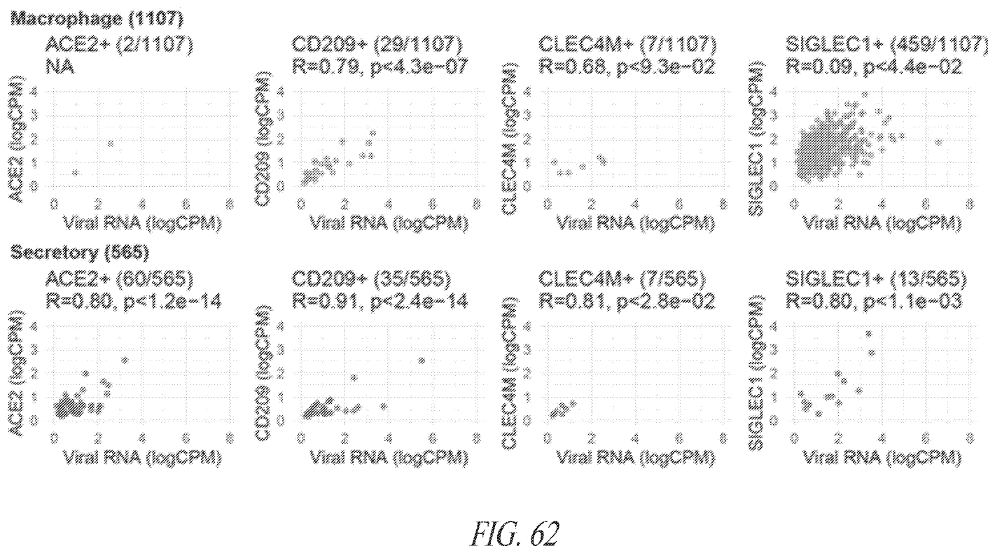

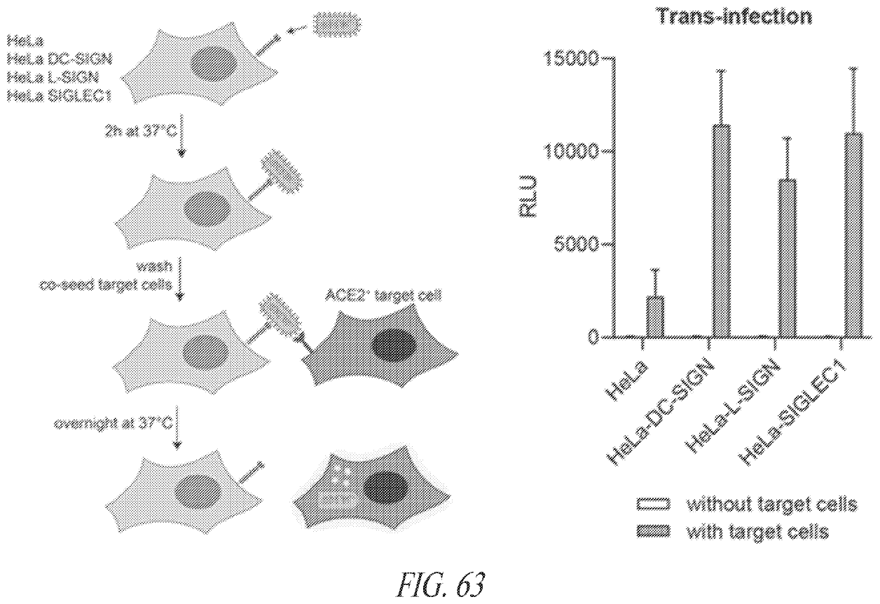

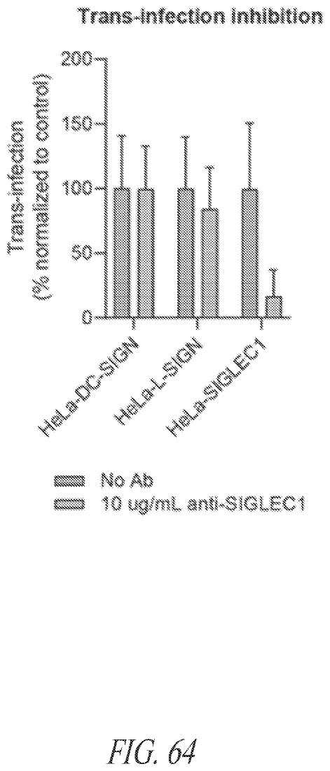

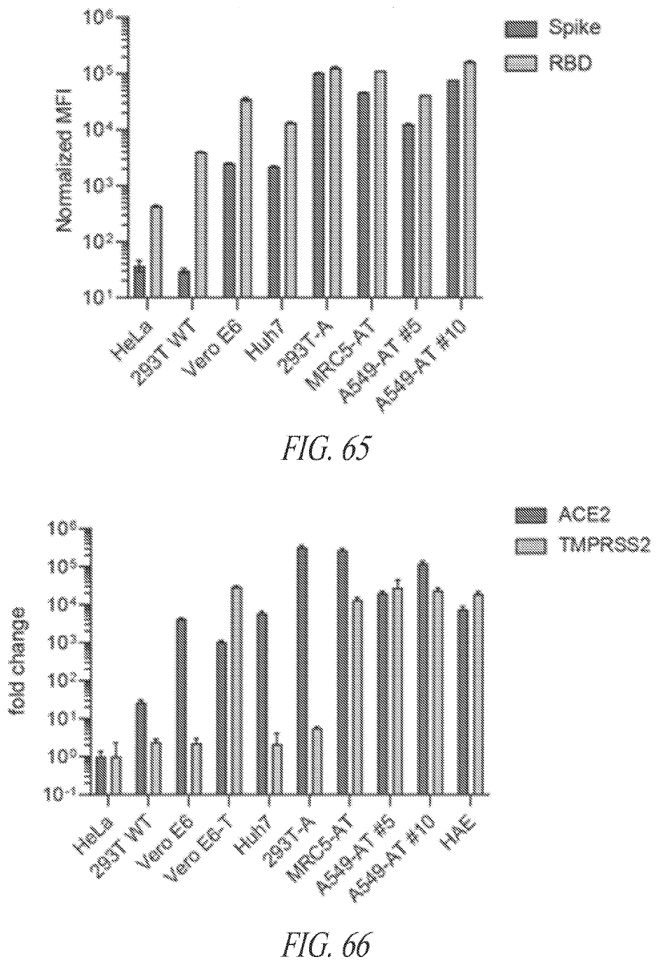

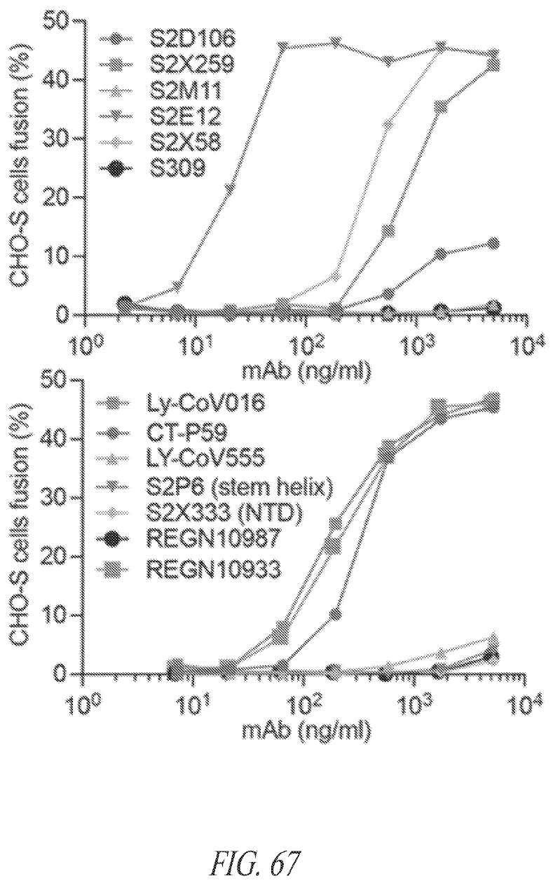

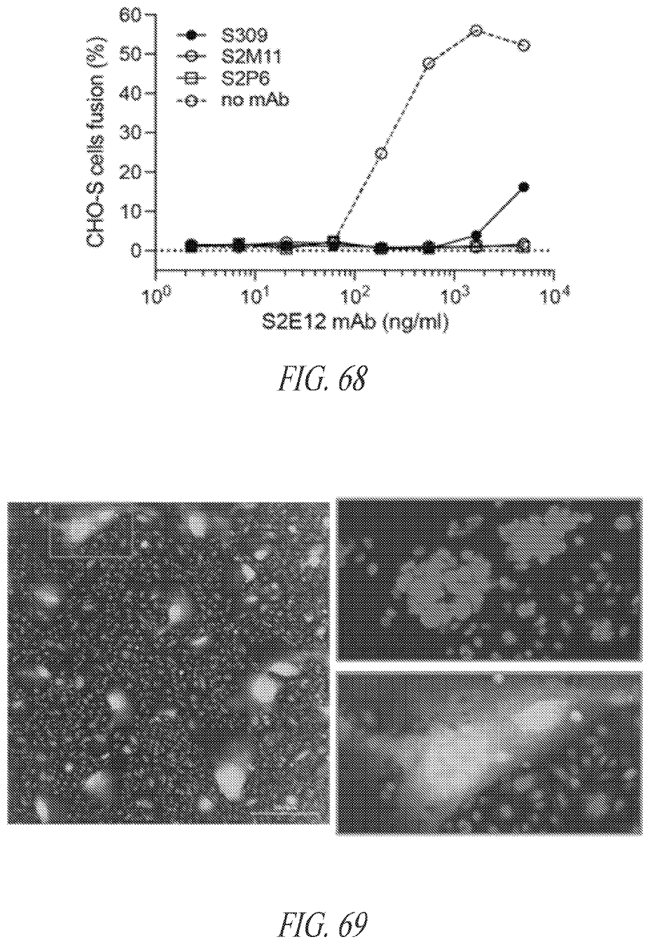

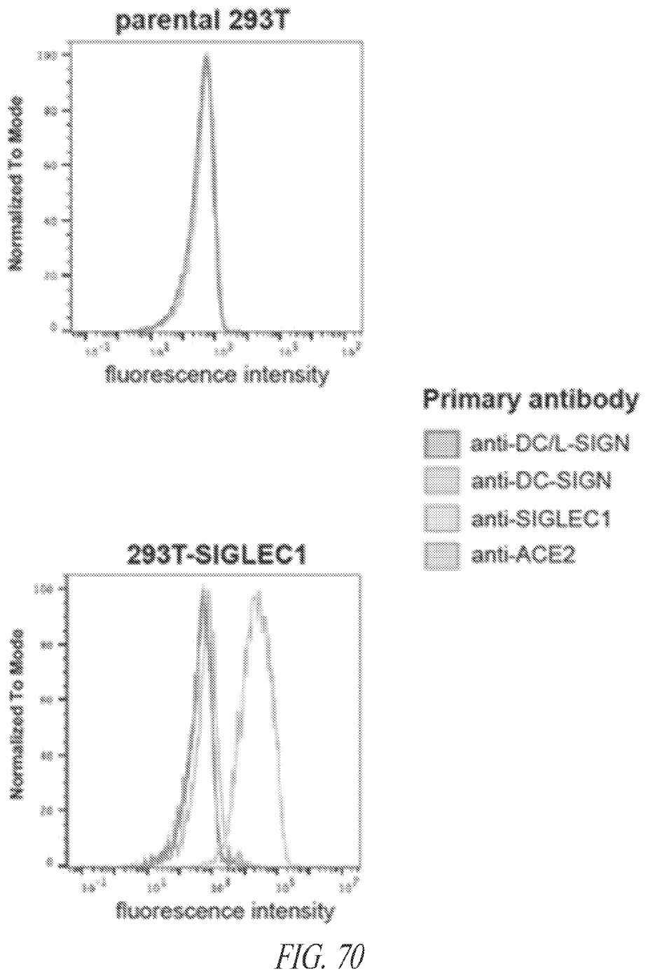

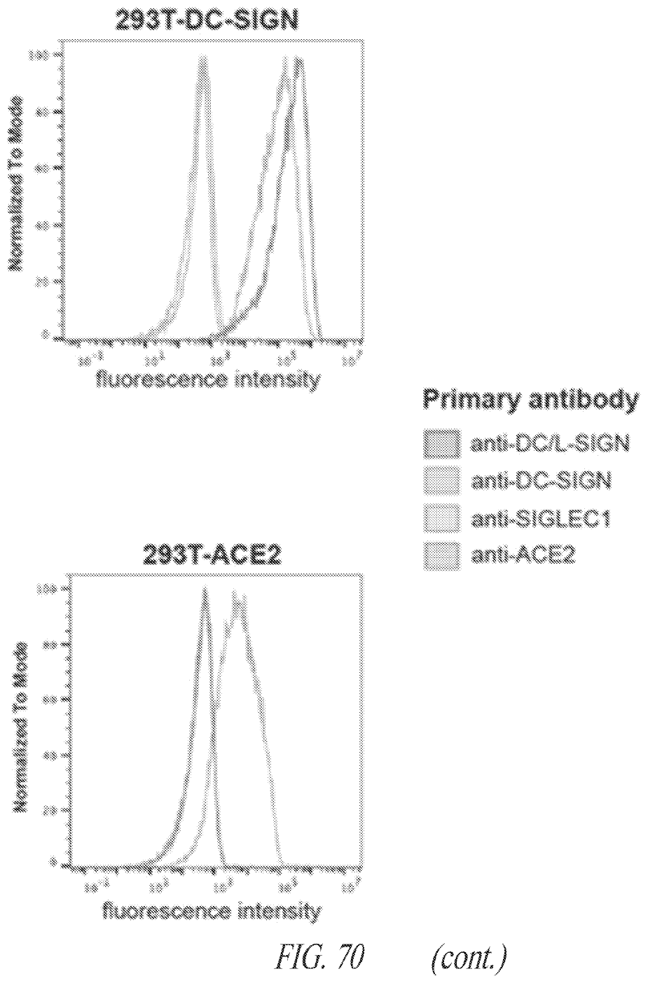

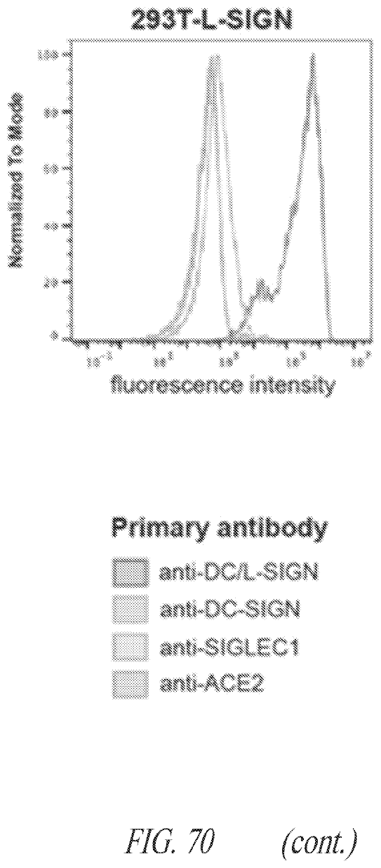

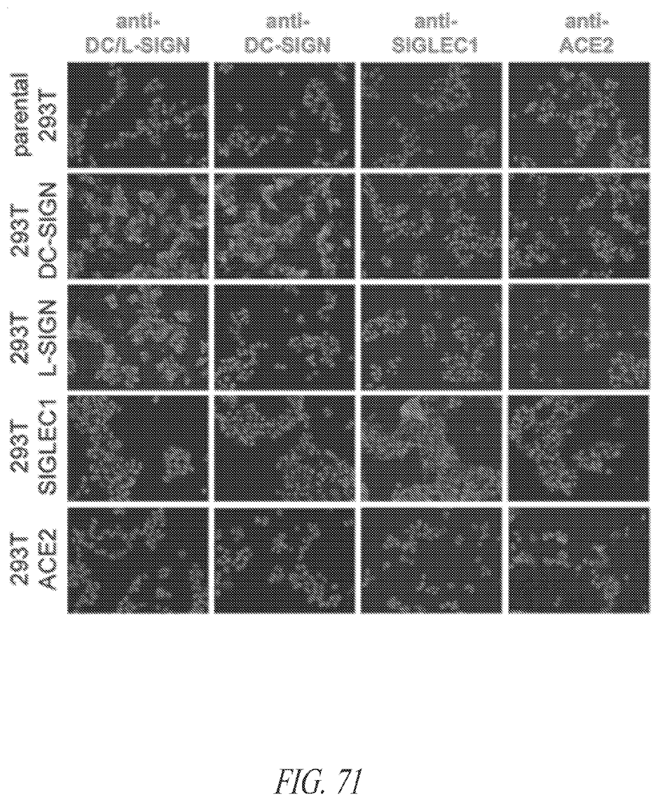

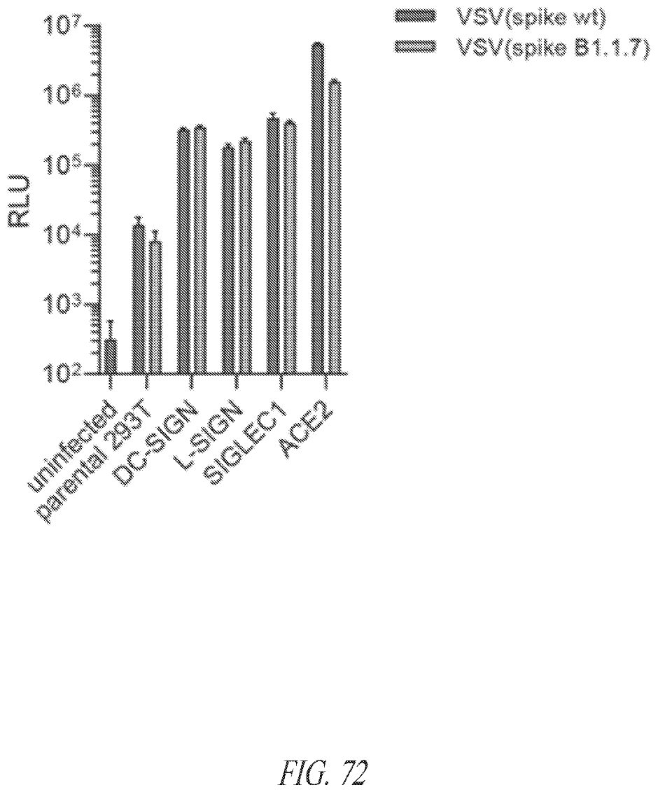

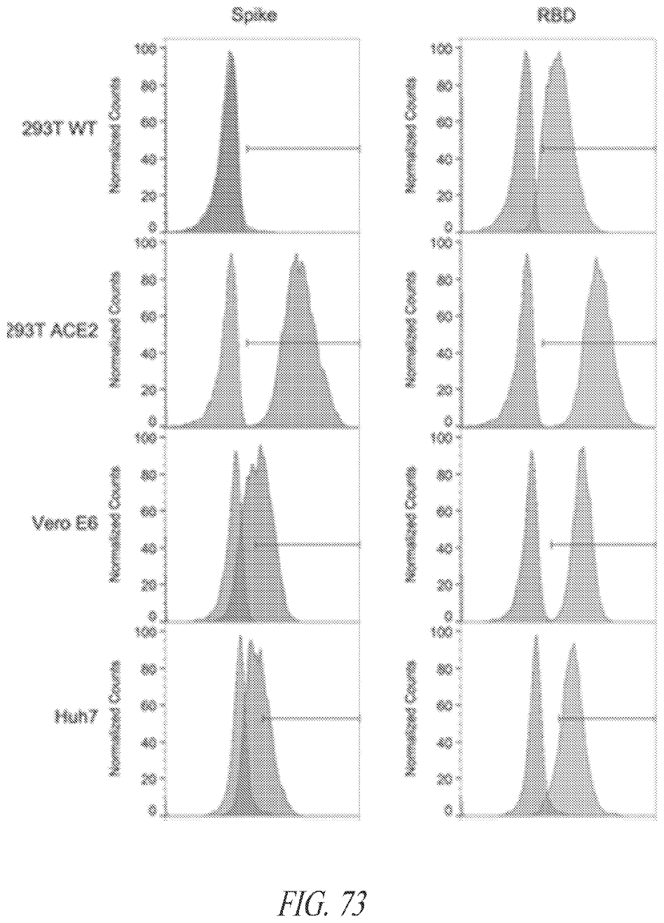

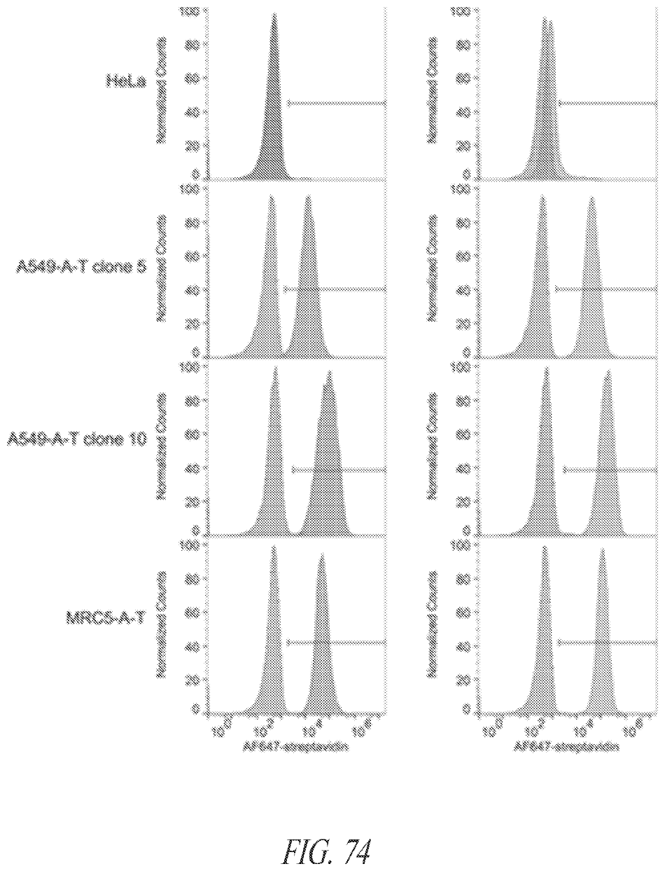

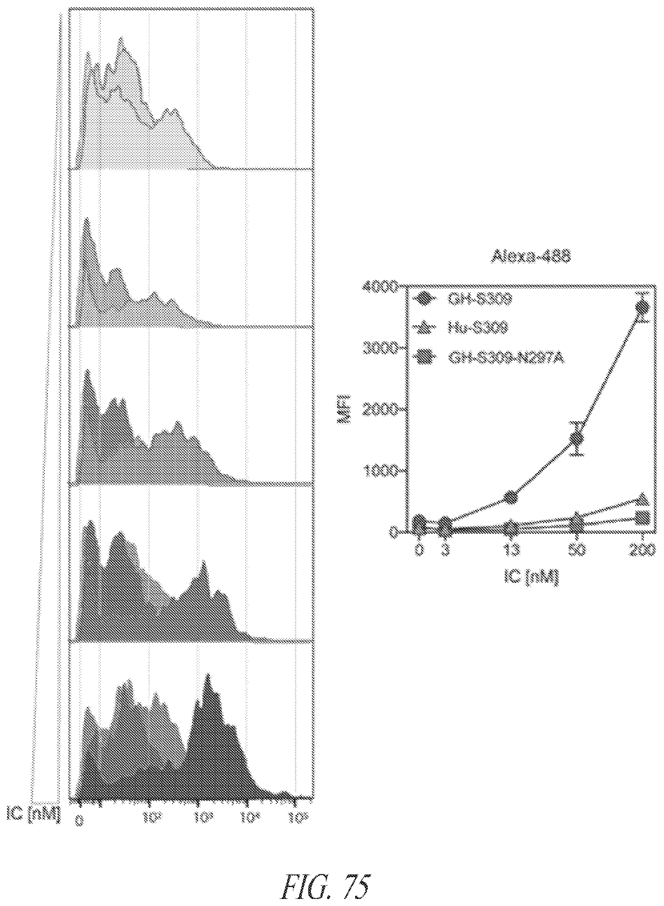

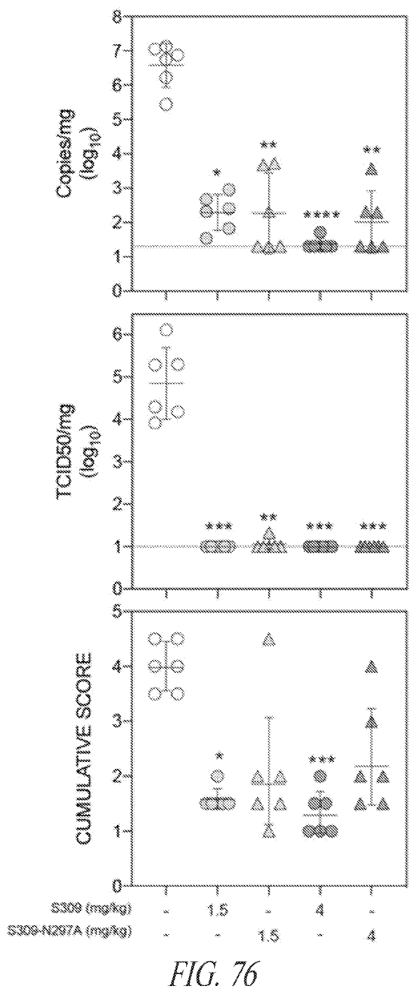

FIGS. 29 and 30 show neutralization of SARS-CoV-2 infection by certain antibodies using a VSV pseudovirus.

FIG. 29 shows data are from one single experiment using triplicate wells of VSV-luc(spike D19) pseudovirus. “LS”=Fc mutations M428L+N434S. IC50 values for the tested antibodies were measured to be 25.69 ng/ml for S309 with wild-type Fc, 1.401 ng/mL for S2E12-LS, 0.9143 ng/mL for S2M11-LS, 3.376 ng/mL for S2D106-LS, 4.085 ng/mL for 409_11_3_v1-LS, 4.446 ng/mL for S2X227-v1-LS, and 2.327 for 409_11_2-LS. Interpolated EC50 and EC90 values for the tested antibodies are shown in Table 16 (ng/ml). All antibodies were expressed as recombinant IgG1.

TABLE 16

| Antibody | EC50 | EC90 |
|---|---|---|
| S309 wt | 25.8 | 121 |
| S2E12-LS | 1.4 | 11.9 |
| S2M11-LS | 1.1 | 14.4 |
| S2D106-LS | 3.6 | 15.4 |
| 409_11_2-LS | 5.3 | 40.9 |
| S2X227-LS | 5.3 | 27.5 |
| 409_11_2-LS | 2.4 | 9.3 |

FIG. 30 shows neutralization of infection by live SARS-CoV-2 by certain antibodies using a VSV pseudovirus. Data are from triplicate wells SARS-CoV-2-luc, MOI 0.1, 6h infection. “LS”=Fc mutations M428L+N434S. IC50 values for the tested antibodies were measured to be 4.844 ng/mL for S2E12-LS, 3.214 ng/mL for S2M11-LS, 4.485 ng/mL for S2D106-LS, 8.233 ng/mL for 409_11_3-LS, 7.061 ng/mL for S2X227-LS, and 84.30 ng/mL for S309. Interpolated EC50 and EC90 values for the tested antibodies are shown in Table 17 (ng/ml).

TABLE 17

| Antibody | EC50 | EC90 |
|---|---|---|
| S2E12-LS | 5 | 30 |
| S2M11-LS | 4 | 11 |
| S2D106-LS | 6 | 22 |
| 409_11_3-LS | 10 | 39 |
| S2X227-LS | 10 | 44 |
| 409_11_2-LS | 5 | 20 |
| S309 | 79 | 289 |

### Example 10

### Competitive Binding of Human Monoclonal Antibodies and RBD to Human ACE2

Competitive binding of recombinant human monoclonal antibodies and human ACE2 to RBD was measured by competition ELISA for monoclonal antibodies S2D4, S2D5, S2D8, S2D10, S2A4, S2D11, S2D15, S2D19, S2D22, S2D25, S2D27, S2D31, S2D32, S2D34, S2D38, S2D39, S2D41, S2D43, S2D47, S2D51, S2D52, S2D53, S2D60.

ELISA plates were coated with recombinant human ACE2 (produced in-house) Coating was carried out with ACE2 at 2 ug/ml in PBS. Plates were incubated overnight at 4° C. and blocking was performed with blocker Casein (1% Casein from Thermofisher) for 1 hour at room temperature.

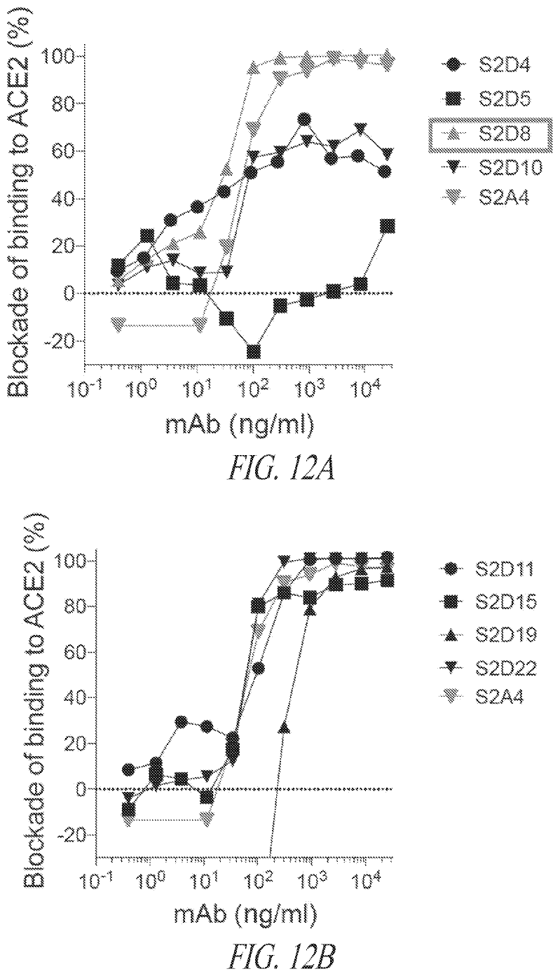

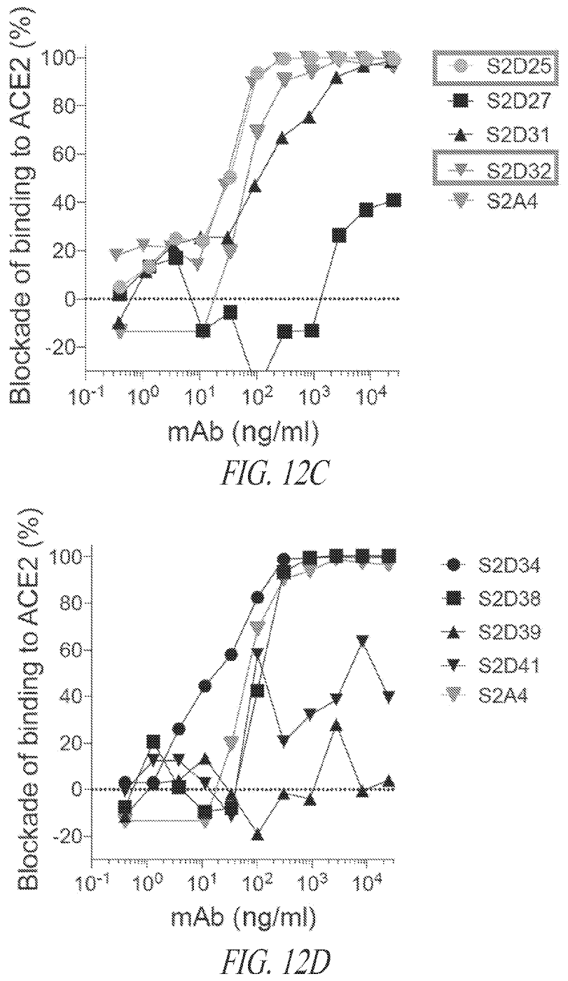

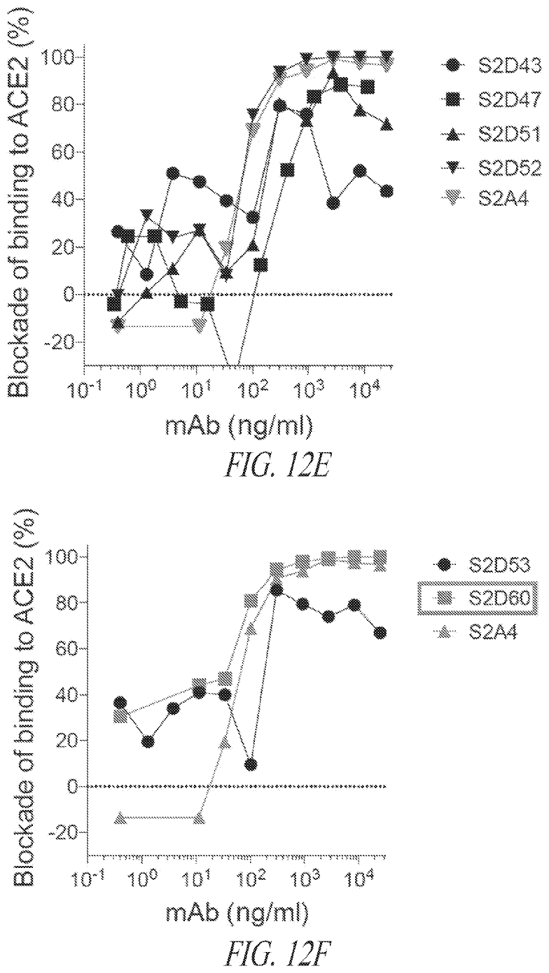

Serial dilutions of monoclonal antibodies were incubated with SARS-CoV-2 RBD at 20 ng/ml (RBD fused with mouse Fc, from Sino Biological) for 30 minutes at 37° C. and then transferred onto the ACE2-coated plates for an additional incubation at room temperature. Plates were washed and binding of RBD to ACE2 was detected using a polyclonal goat anti-mouse Fc-AP antibody (Southern Biotech). After an additional wash, AP substrate pNPP (Sigma) was added and plates were incubated at 20 minutes at room temperature before measuring adsorbance at 405 nm with a spectrophotometer (Powerwave340 Biotek). Results are shown in FIGS. 12A-12F.

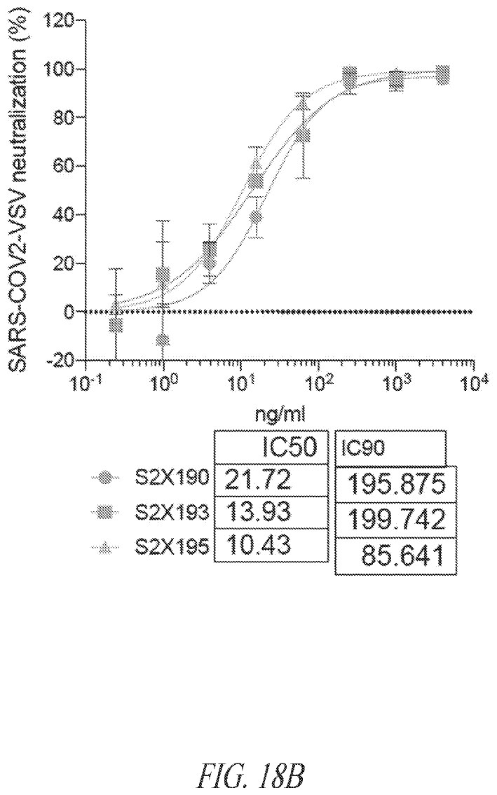

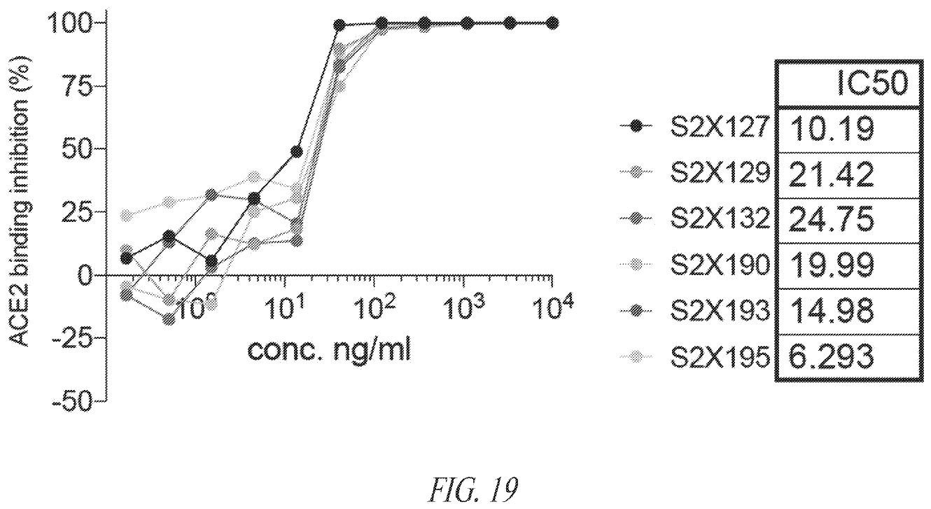

Further assays were carried out using similar methods for monoclonal antibodies S2X127, S2X129, S2X132, and S2X190. Results are shown in FIG. 19. Calculated IC50 values are shown on the right of the figure.

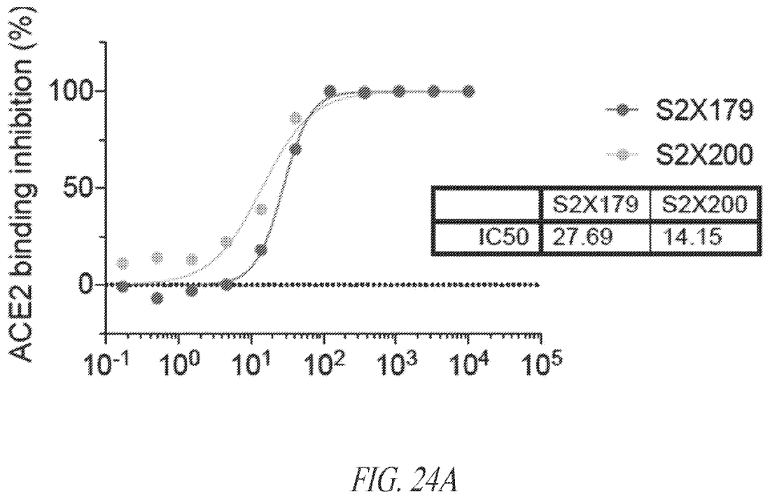

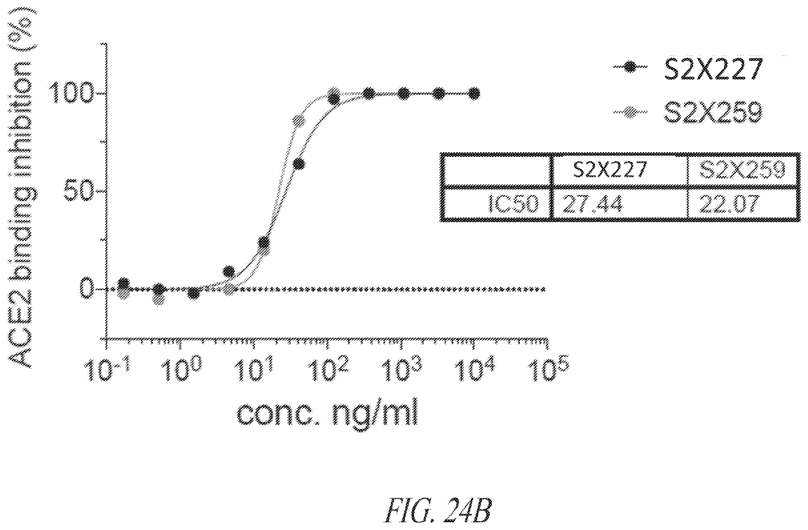

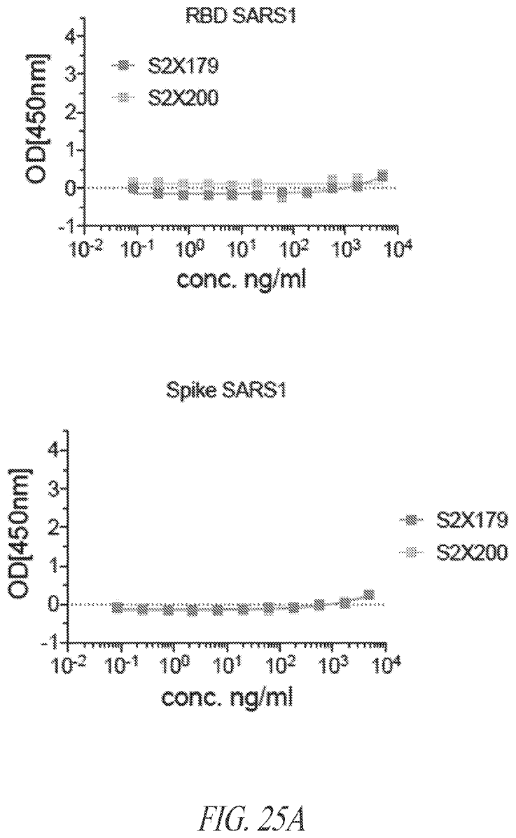

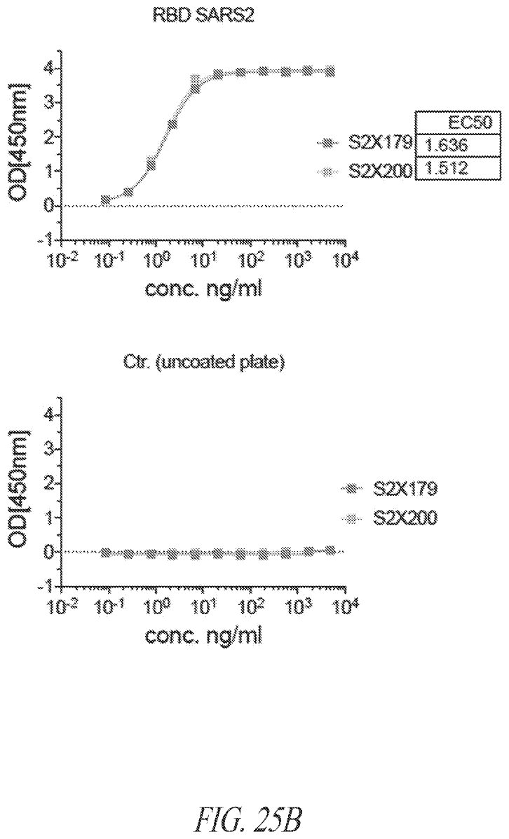

Competitive binding for monoclonal antibodies S2X200, S2X227, S2X259, and comparator antibody S2X179 with human ACE2 to RBD was determined using similar methods. Results are shown in FIGS. 24A-24B. Calculated IC50 values are shown on the right of each figure.

### Example 11

### Competitive Binding of Human Monoclonal Antibodies to RBD of SARS-CoV-2

Competitive binding of pairs of monoclonal antibodies to SARS-CoV-2 RBD was measured to distinguish binding sites of the antibodies.

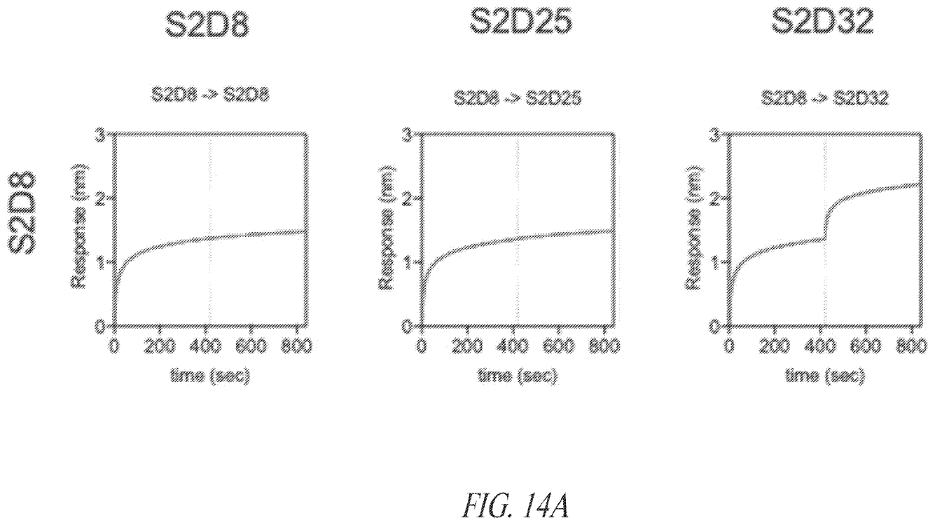

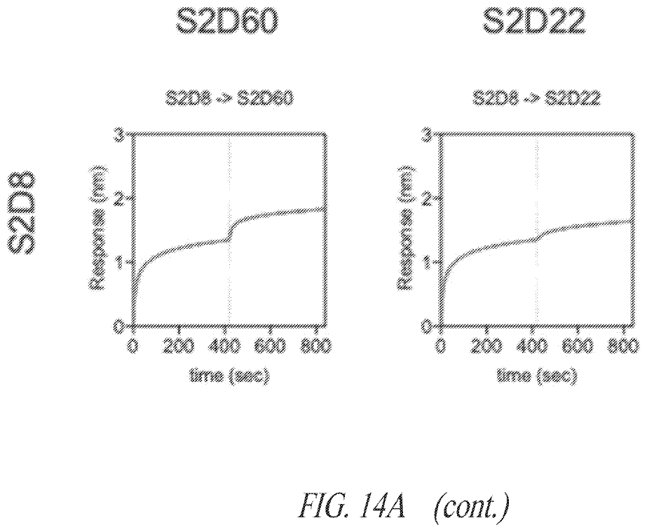

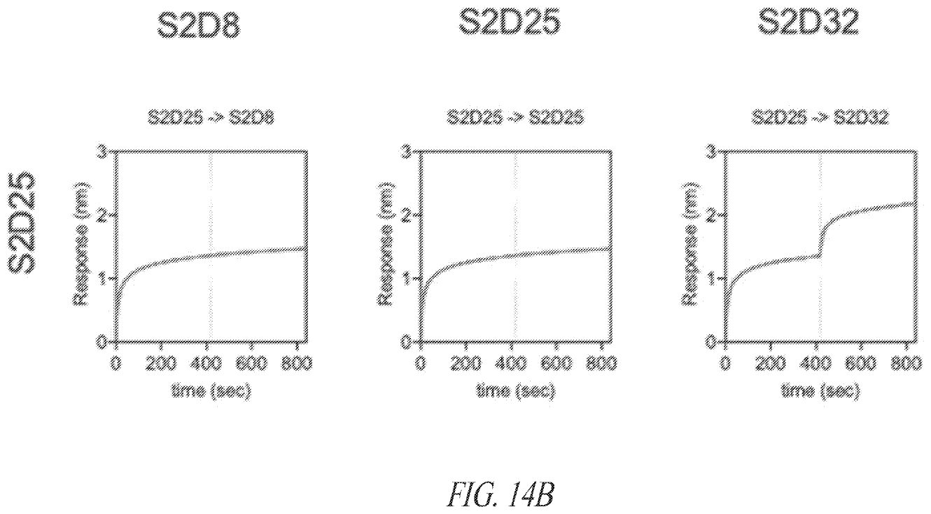

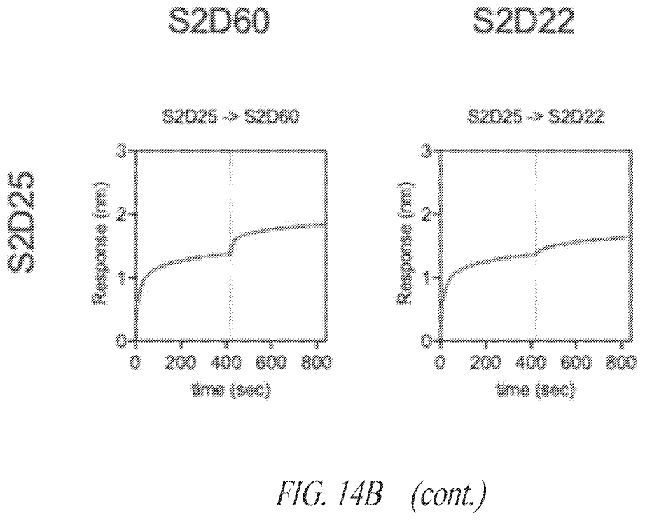

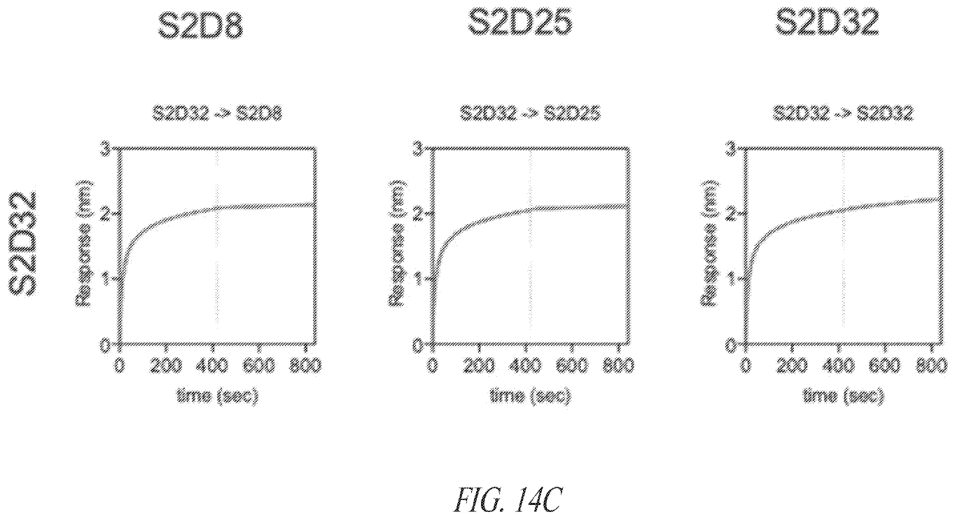

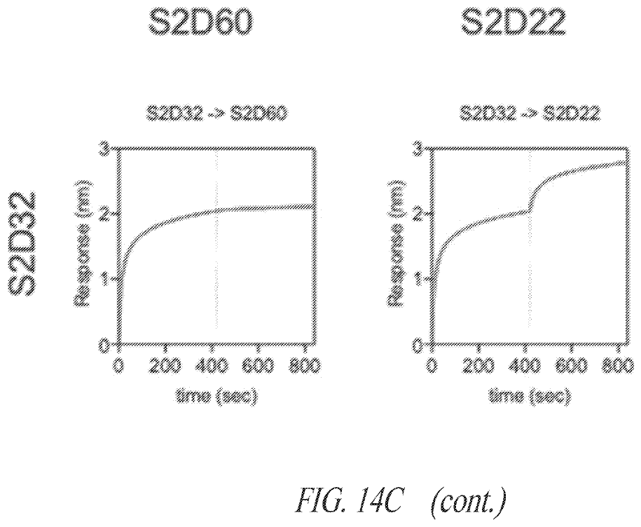

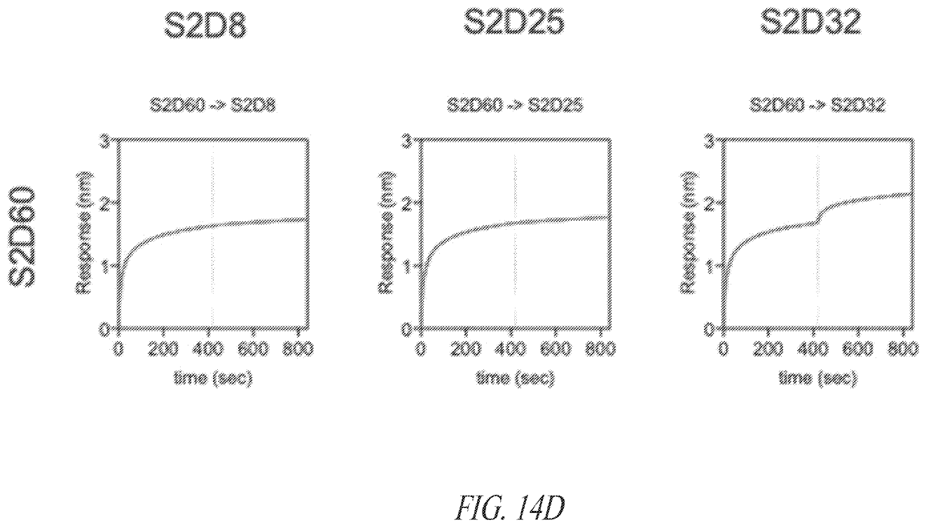

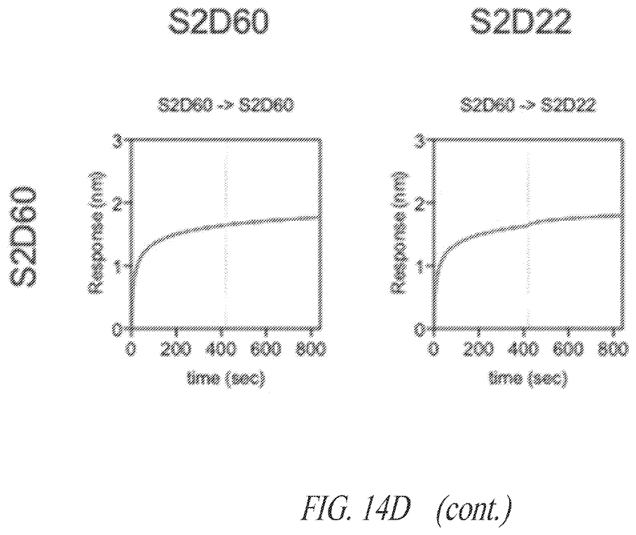

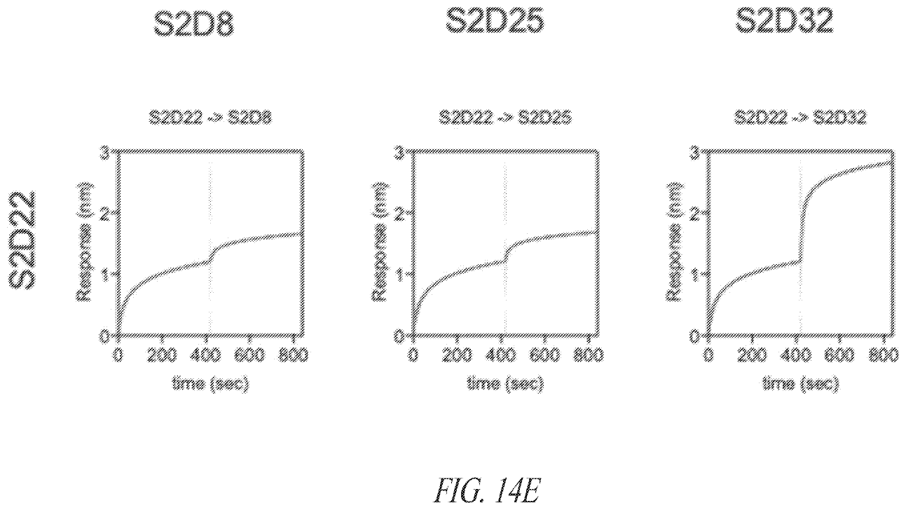

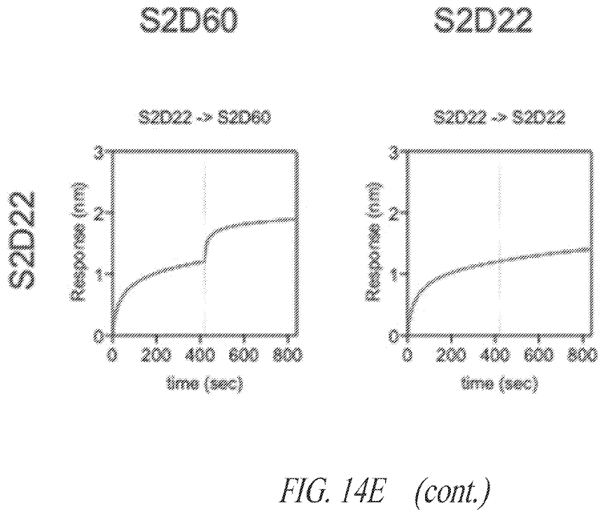

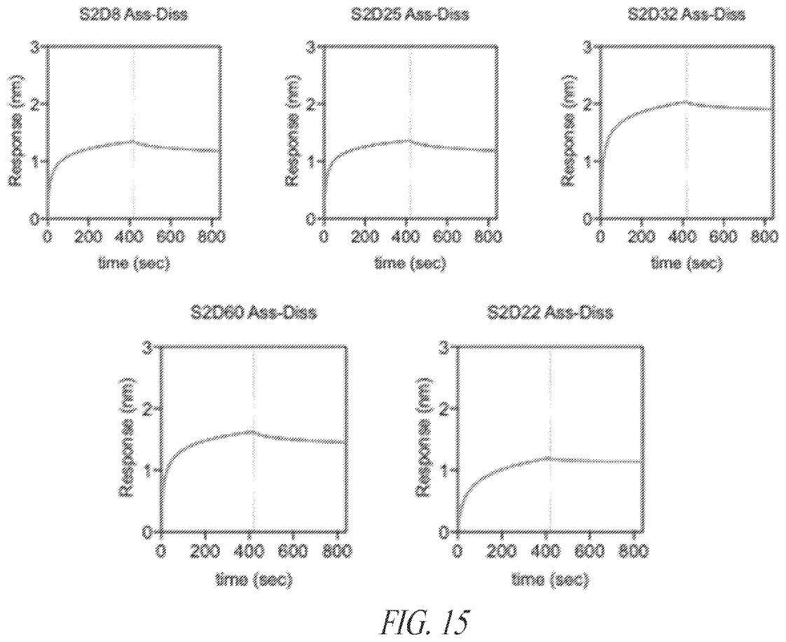

Strepavidin biosensors (Pall ForteBio) were used to immobilize anti-Strep Tag II antibody at 3 ug/ml (clone 5A9F9, Biotin, LabForce AG, Muttenz CH), after a hydration step for 10 min with Kinetics Buffer (KB; 0.01% endotoxin-free BSA, 0.002\hat{ } Tween-20, 0.005% NaN3 in PBS). SARS-CoV-2 RBD with a Strep Tag II (produced in-house) was then loaded for 6 min at a concentration of 4 μg/ml in KB. The first antibody was allowed to associate for a period of time, then the second antibody was allowed to associate for a period of time. Results are shown in FIGS. 14A-14E. The dashed vertical lines in each graph indicate the switch from the first antibody, indicated on the left, to the second antibody, indicated on top.

### Example 12

### Binding of Human Monoclonal Antibodies to RBD Using Octet

Binding affinity of monoclonal antibodies S2D8, S2D25, S2D32, S2D60, and S2D22 for SARS-CoV-2 RBD was tested using Octet. His-tagged RBD of SARS-CoV-2 was loaded at 3 μg/ml in kinetics buffer (KB) for 15 minutes onto anti-HIS (HIS2) biosensors (Molecular Devices, ForteBio). Association of monoclonal antibodies was performed in KB at 15 μg/ml for 5 minutes. Dissociation in KB was measured for 10 minutes. Octet Red96 (ForteBio) equipment was used.

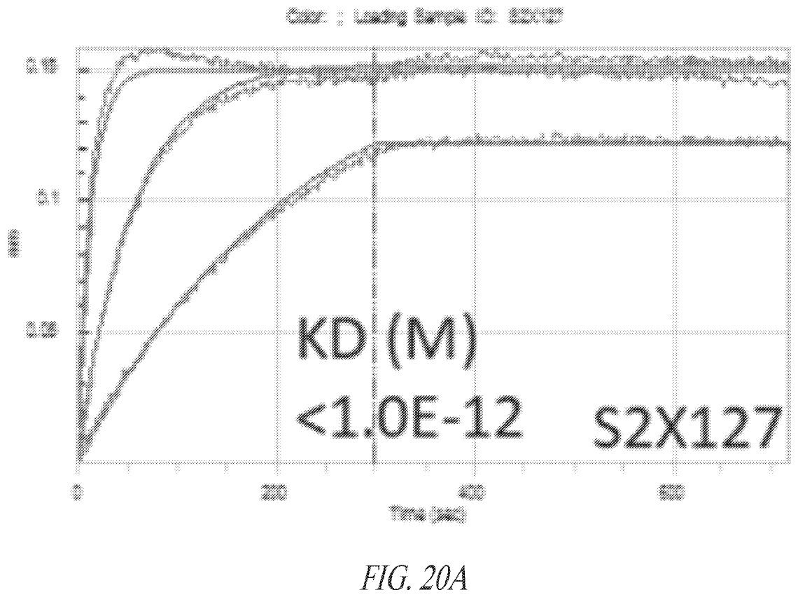

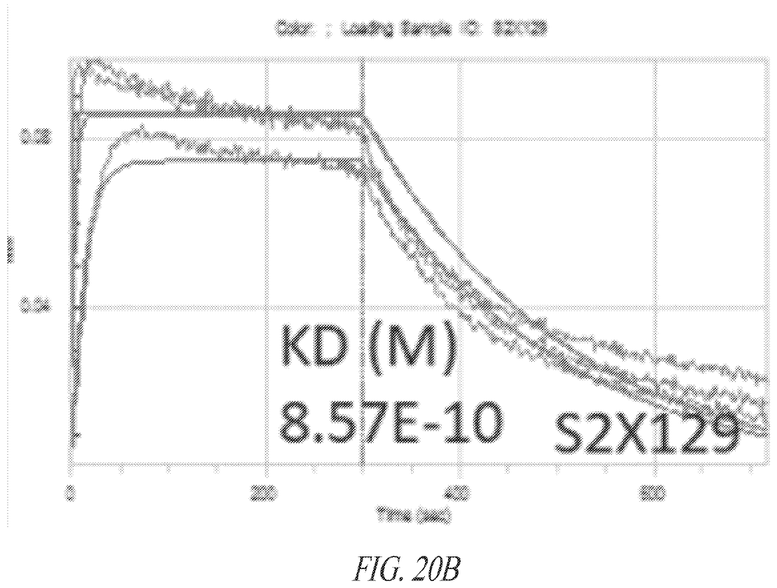

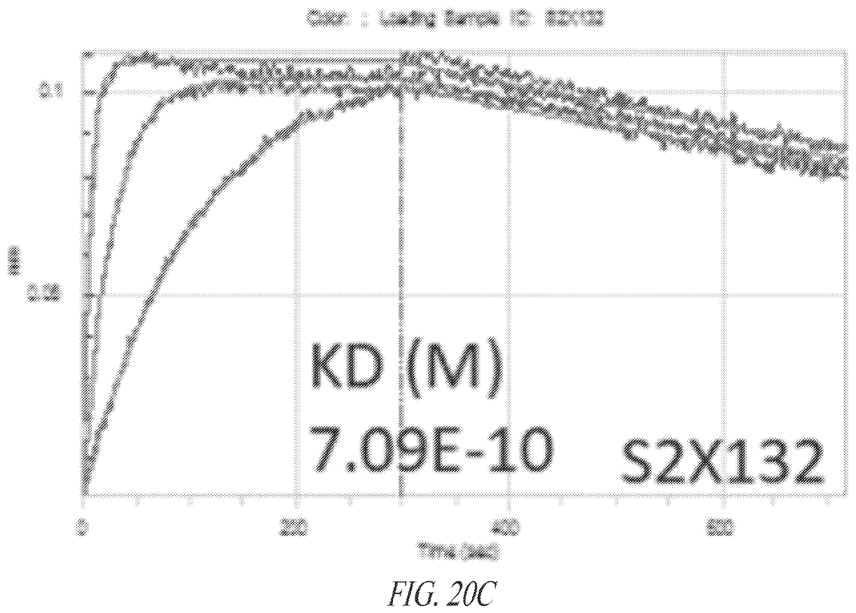

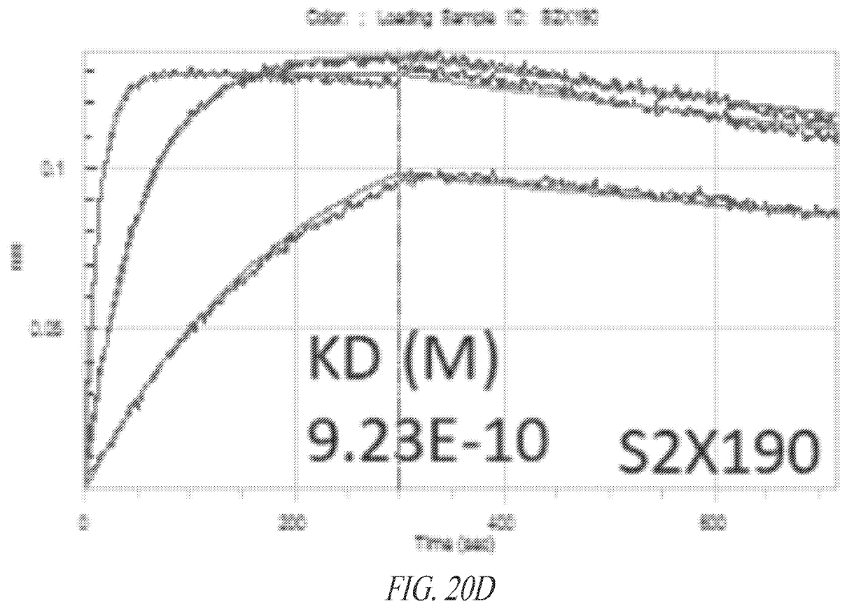

Binding affinity and avidity of monoclonal antibodies S2X127, S2X129, S2X132, and S2X190 for SARS-CoV-2 RBD was measured by Octet. Antibody was loaded on Protein A pins at 2.7 μg/ml. SARS-CoV-2 RBD was loaded for 5 minutes at 6 μg/ml, 1.5 μg/ml, or 0.4 μg/ml. Dissociation was measured for 7 minutes. The vertical dashed line in each figure indicates the start of the dissociation phase. Results are shown in FIGS. 20A-20D.

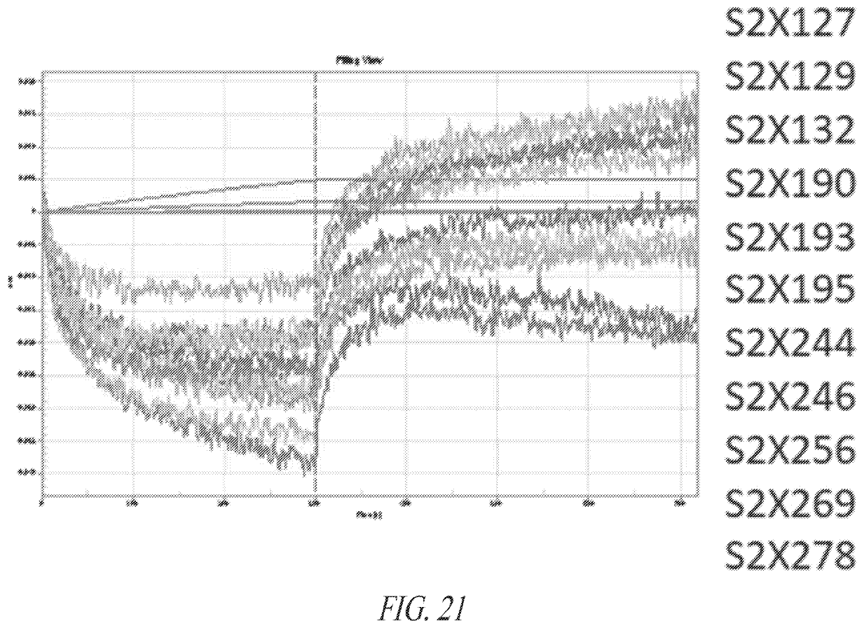

Binding affinity and avidity of monoclonal antibodies S2X127, S2X129, S2X132, and S2X190, along with seven comparator antibodies, to SARS-CoV RBD was also measured by Octet. Antibody was loaded on Protein A pins at 2.7 μg/ml. SARS-CoV RBD was loaded for 5 minutes at 6 μg/ml. Dissociation was measured for 7 minutes. The vertical dashed line in each figure indicates the start of the dissociation phase. Results are shown in FIG. 21, along with results for seven comparator antibodies.

### Example 13

### Quantitative Epitope-Specific Serology of SARS-CoV-2 Spike Protein

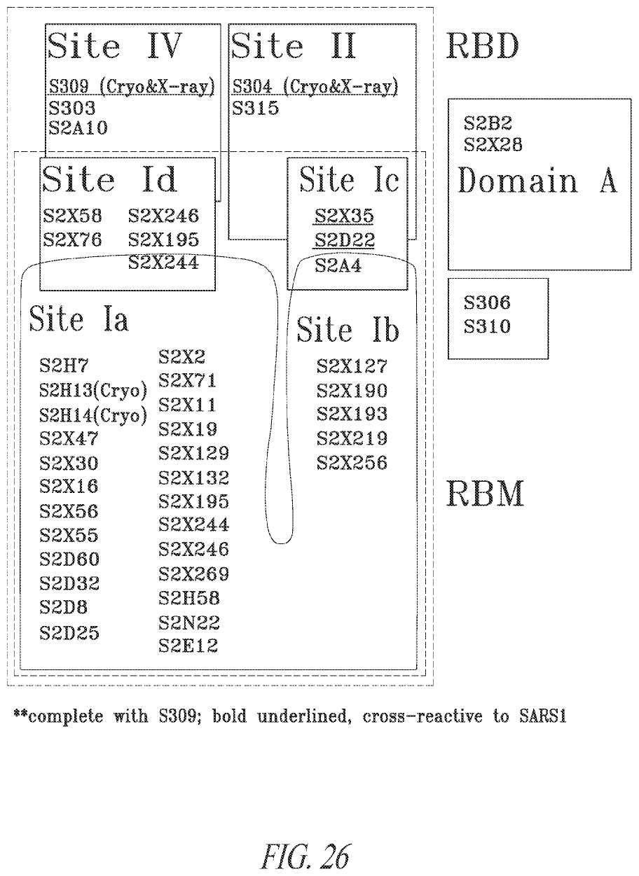

SARS-CoV-2 Spike protein antibody binding was analyzed by antibody competition assays, cryo-EM data, and crystallography data. From this analysis, Spike RBD antigenic Sites Ia, Ib, Ic, Id, II, and IV were identified. A map showing these sites and representative antibodies that bind within each site is shown in FIG. 26.

### Example 14

### Development of S2D5, S2D25, S2D32, and S2D60 rIgG Variants

Recombinant IgG1 antibodies were developed using the VH and VL sequences of monoclonal antibodies S2D5, S2D25, S2D32, and S2D60. The combinations are produced as indicated in Table 18. Each of the variant antibodies is produced by transient transfection and expression of a plasmid vector encoding the recombinant antibody in HD 293F cells (GenScript). Cells are harvested on day 4 and IgG expression is validated by Western blot and protein A titer analysis.

TABLE 18

| | | | VL (WT | |
|---|---|---|---|---|
| | | VH SEQ | or | VL SEQ |
| mAb | VH (WT or variant) | ID | variant) | ID |
| S2D8-11 | WT | 210 | WT | 214 |
| S2D8-21 | D62E | 252 | WT | 214 |
| S2D8-31 | W101F | 253 | WT | 214 |
| S2D8-41 | W101F-D62E | 255 | WT | 214 |
| S2D8-51 | I105A-S63A | 256 | WT | 214 |
| S2D8-61 | W101F-I105A-S63A | 258 | WT | 214 |
| S2D8-71 | W101Y-I105A-S63A | 260 | WT | 214 |
| S2D25-11 | WT | 220 | WT | 224 |
| S2D25-21 | D62E | 262 | WT | 224 |
| S2D25-31 | W101F | 264 | WT | 224 |
| S2D25-41 | W101F-D62E | 266 | WT | 224 |
| S2D25-51 | W101F-S63A-I31S | 267 | WT | 224 |
| S2D25-61 | I31S-I105A-S63A | 270 | WT | 224 |
| S2D25-71 | W101Y-S63A-I31S | 272 | WT | 224 |
| S2D32-11 | WT | 232 | WT | 236 |
| S2D32-21 | W103F | 274 | WT | 236 |
| S2D32-12 | WT | 232 | W94F | 282 |
| S2D32-22 | W103F | 274 | W94F | 282 |
| S2D32-31 | C105S-C110S | 276 | WT | 236 |
| S2D32-41 | C105P-C110P | 278 | WT | 236 |
| S2D32-51 | C105A-C110A | 280 | WT | 236 |
| S2D60-11 | WT | 242 | WT | 246 |
| S2D60-21 | W50F-T61A | 284 | WT | 246 |
| S2D60-31 | W50F-N59Q | 286 | WT | 246 |
| S2D60-41 | T61A-G109A | 288 | WT | 246 |
| S2D60-51 | W50F-G109A-T61A | 291 | WT | 246 |
| S2D60-61 | W50Y-D108E-N59Q | 292 | WT | 246 |
| S2D60-71 | C101S-C106S-T61A | 295 | WT | 246 |
| S2D60-82 | T61A | 297 | W97F | 302 |
| S2D60-92 | N59Q | 298 | W97F | 302 |
| S2D60-102 | N59D | 300 | W97F | 302 |

### Example 15

### Testing of Antibodies Against Multiple Sarbecoviruses

Properties of monoclonal antibodies S2X259 and S2D22 with respect to a variety of sarbecoviruses were tested.

Binding of antibody S2X259 (VH amino acid sequence set forth in SEQ ID NO.:408 (HCDRs of SEQ ID NOs.:409-411); VL amino acid sequence set forth in SEQ ID NO.:412 (LCDRs of SEQ ID NOs.:413-415)) to the spike protein RBD of sarbecoviruses from clade 1a, clade 1b, clade 2 (non-ACE2-utilizing Adian sarbecoviruses), and clade 3 (African and European sarbecoviruses) was investigated by yeast surface-display assay. Clade 1a viruses tested were 12 SARS-CoV strains, LYRa11, WIV1, Rs7327, Rs4231, RsSHC014, and Rs4084. Clade 1b viruses tested were SARS-CoV-2, RaTG13, GD-Pangolin, and GX-Pangolin. Clade 2 viruses tested were Rf4092, RbYN02, YN2013, ZC45, ZXC21, Rf1, JL2012, 273-2005, HeB2013, HuB2013, Rs4247, Longquan-140, HKU3-1, GX2013, Shaanxi2011, 279-2005, As6526, Yunnan2011, Rs4237, Rs4081, and Rp3. Clade 3 viruses tested were BM48-31 and BtKY72. S2X259 was found to bind to the spike protein RBD of all tested clade 1a, clade 1b, and clade 3 sarbecoviruses tested, while showing weak or no binding to clade 2 sarbecoviruses.

Binding of antibodies S2X259 and S2D22 to spike proteins from different sarbecoviruses was measured by flow cytometry (FACS), and binding to spike protein RBDs from different sarbecoviruses was measured by enzyme-linked immunoabsorbant assay (ELISA). Results are shown in Table 19 (“POS”=positive for binding; “NEG”=negative for binding).

TABLE 19

| | | | S2X259 | S2D22 |
|---|---|---|---|---|
| FACS | Clade 1a | SARS-CoV | POS | POS |
| Binding | Clade 1b | SARS-CoV-2 | POS | POS |
| | | PANG/GD | POS | POS |
| | | PANG/GX | POS | POS |
| | | RATG13 | POS | POS |
| | Clade 2 | ZXC21 | POS | POS |
| | | ZC45 | POS | POS |
| | | RMYN02 | POS | POS |
| | | YN2013 | POS | NEG |
| | Clade 3 | BGR2008 | POS | NEG |
| | | BtkY72 | POS | POS |
| ELISA | Clade 1a | SARS-CoV | 28.54 | 42.21 |
| Binding | Clade 1b | SARS-CoV-2 | 34.34 | 35.27 |
| EC50 | | PANG/GD | 36.16 | 49.06 |
| (ng/ml) | | PANG/GX | 28.13 | 43.2 |
| | | RATG13 | 40.13 | 45.11 |
| | Clade 2 | ZC45 | NEG | 48.48 |
| | Clade 3 | BGR2008 | 30.79 | NEG |
| | | BtkY72 | 37.6 | 52.86 |

Antibody S2X259 was also found to bind to spike RBD of further clade 2 viruses with EC50 values in the following ranges (ng/ml): Anlong1 12: 100-1000, YN2013: 1-50, SC2018: 1-50, SX2011: 1-50. Antibody S2D322 was found to bind to spike RBD of additional clade 2 viruses with EC50 values in the following ranges (ng/ml): Anlong1 12: 1-50, YN2013: 100-1000. S2D322 did not bind to RBD of cade 2 viruses SC2018 or SX2011.

Affinity of antibodies S2X259 and S2D322 for spike protein RBD of sarbecoviruses from various clades was measured by BLI. Results are shown in Table 20, which lists the measured KD in M.

TABLE 20

| | | S2X259 | S2D22 |
|---|---|---|---|
| Clade 1a | SARS-CoV | 5.37E−10 | 5.35E−08 |
| | WIV-1 | 5.70E−10 | 4.00E−08 |
| Clade 1b | SARS-CoV-2 | 1.00E−12 | 5.21E−09 |
| Clade 2 | ZC45 | Not detected | 4.14E−08 |
| Clade 3 | BGR2008 | 1.04E−09 | Not detected |

Neutralizing activity of antibodies S2X259 and S2D22 against VSV pseudotyped with SARS-CoV spike protein, MLV pseudotyped with SARS-CoV-2 spike protein, VSV pseudotyped with PANG/GD19 spike protein, and VSV pseudotyped with PANG-GX17 spike protein was measured. IC50 values are shown in Table 21 (μg/ml).

TABLE 21

| | S2X259 | S2D22 |
|---|---|---|
| VSV-SARS-CoV pp | 0.18855 | 3.634 |
| MLV-SARS-CoV-2 pp | 0.046 | 1.033 |
| VSV-PANG/GD19 pp | 0.1792 | 0.09548 |
| VSV-PANG/GX17 pp | 1.322 | 0.284 |

Additional studies were performed to investigate the ability of antibodies S2X259 and S2D22 to block binding of ACE2 to spike protein RBD of SARS-CoV and SARS-CoV-2 and to induce FcγR activation. Antibody blocking of binding of RBDs to immobilized human recombinant ACE2 ectodomain was measured by ELISA. Antibody-dependent activation of human FcγRs was performed with a bioluminescent reporter assay. ExpiCHO cells transiently expressing full-length wild-type SARS-CoV-2 S (target cells) or full-length prefusion stabilized SARS-CoV-2 S, which harbours the 2P mutation and S1/S2 furin cleavage site mutation (RRARS to SGAG) were incubated with different amounts of mAbs. After a 15-minute incubation, Jurkat cells stably expressing FcγRIIIa receptor (V158 variant) or FcγRIIa receptor (H131 variant) and NFAT-driven luciferase gene (effector cells) were added at an effector to target ratio of 6:1 for FcγRIIIa and 5:1 for FcγRIIa. Signaling was quantified by the luciferase signal produced as a result of NFAT pathway activation. Luminescence was measured after hours of incubation at 37° C. with 5% C02 with a luminometer using the Bio-Glo-TM Luciferase Assay Reagent according to the manufacturer's instructions (Promega). Results are shown in Table 22.

TABLE 22

| | | S2X259 | S2D22 |
|---|---|---|---|
| ACE2/RBD blocking | SARS-CoV | +++ | + |
| | SARS-CoV-1 | +++ | + |
| FcγR activation | FcγRIIIA | +/− | ++ |
| | FcγRIIA | − | − |

### Example 16

### Design and Testing of Additional Variant Antibodies

S2X259 variant antibodies were generated and tested for binding against sarbecovirus RBDs using BLI. Parental S2X259 (VH of SEQ ID NO.:408, VL of SEQ ID NO.:412) was also tested. Data are summarized in Table 23.

TABLE 23

| V-region SEQ | | | | | | | |
|---|---|---|---|---|---|---|---|
| ID NOs. (aa) of | | | | | | | |
| certain S2X259 | | | | | | | |
| antibodies | | KD [M] | | | | | |
| VH | VL | RBD2-D | SARS1 | BGR2008 | ZC45 | WIV-1 | SC2018 |
| 408 | 412 | 8.74E−11 | 8.62E−10 | 1.24E−09 | n.d. | 6.10E−10 | 6.93E−09* |
| 408 | 442 | 6.05E−11 | 3.97E−10 | 5.22E−10 | n.d. | 3.45E−10 | 5.55E−09 |
| 408 | 443 | 8.28E−11 | 5.21E−10 | 7.35E−10 | n.d. | 4.22E−10 | 7.94E−09 |
| 408 | 444 | 6.42E−11 | 1.34E−09 | 1.96E−09 | n.d. | 9.86E−10 | 6.00E−09* |
| 408 | 445 | 7.64E−11 | 5.30E−10 | 8.59E−10 | n.d. | 4.46E−10 | 8.36E−09 |
| 446 | 412 | 5.72E−10 | 5.58E−09 | 4.95E−09 | n.d. | 3.06E−09 | 1.20E−09* |
| 448 | 412 | 5.84E−10 | n.d. | n.d. | n.d. | n.d. | n.d. |

*no optimal fitting

These S2X259 antibodies were also tested for binding (ELISA) to Clade 1a (SARS-CoV, WIV1), Clade 1b (SARS-CoV-2, RatG13, PangGD, PangGX), Clade 2 (Anlong1 12, YN2013, SC2018, SX2011, ZC45), Clade 3 (BtKY72, BGR2008, N501Y) and SARS-CoV-2 mutant (N501Y, Y453F, N439K, K417V, N501Y-K417N-E484K) RBDs by ELISA. With the exception of the S2X259 variant having the VH of SEQ ID NO.:448 and the VL of SEQ ID NO.:412 (which did not detectably bind to SARS-CoV, WIV1, RatG13, PanGX, Anlong112, YN2013, SC2018, SX2011, ZC45, BtKY472, and BGR2008), the S2X259 antibodies all bound to all of the tested RBDs with an EC50 of between 1 and 1,000 ng/mL, with most EC50 values between 1 and 100 ng/mL.

The S2X259 antibodies were also evaluated for their ability to neutralize infection by MLV pseudotyped with SARS-CoV2 S protein. IC50 values were as shown in Table 24.

TABLE 24

V-region SEQ ID NOs. (aa) of certain
S2X259 antibodies

| VH SEQ ID NO.: | VL SEQ ID NO.: | IC50 (ng/mL) |
|---|---|---|
| 408 | 412 | 230.8 |
| 408 | 442 | 190.0 |
| 408 | 443 | 97.48 |
| 408 | 444 | 167.1 |
| 408 | 445 | 189.7 |
| 446 | 412 | 320.5 |
| 448 | 412 | 13.38 |

Additionally, the variant antibody having the VH amino acid sequence of SEQ ID NO.:408 and the VL amino acid sequence of SEQ ID NO.:412, expressed as rIgG1 with M428L/N434S mutations in the Fc, had an IC50 of 184.2 ng/mL. For comparison, the following antibodies, also expressed as rIgG1 with M428L/N434S mutations in Fc, were tested, providing the following IC50 values: S2K15=570.3 ng/mL; S2H90=115.1 ng/mL; S2H94=1671 ng/mL; S2H97=2761 ng/mL.

S2X259 antibodies were also evaluated for their ability to neutralize infection by VSV pseudotyped with SARS-CoV. Antibodies S309 (VH SEQ ID NO.:172; VL SEQ ID NO.: 176) and S2H94 were also tested. IC50 values were as shown in Table 25.

TABLE 25

V-region SEQ ID NOs. (aa) of antibodies

| VH SEQ ID NO.: | VL SEQ ID NO.: | IC50 (ng/mL) |
|---|---|---|
| 408 | 412 | 80.6 |
| 408 | 442 | 53.1 |
| 408 | 443 | 35.3 |
| 408 | 444 | 81.1 |
| 408 | 445 | 36.0 |
| (S2H94) | (S2H94) | 76.9 |
| (S309) | (S309) | 41.0 |

S2X259 antibodies were also evaluated for their ability to neutralize infection by VSV pseudotyped with SARS-CoV, GD19, GX17, WIV-1, or SARS-CoV-2. Antibodies S309 (VH SEQ ID NO.:172; VL SEQ ID NO.:176) and S2H94 were also tested. All S2X259 antibodies except for the variant having the VH of SEQ ID NO.:448 and the VL of SEQ ID NO.:412 neutralized all of the pseudotyped VSV viruses with an IC50 value of less than 450 ng/mL, and in most cases less than 300 or less than 200 ng/mL. In this assay, S2H94 did not neutralize WIV-1, SARS-CoV-2, or GX17. In this assay, S309 did not neutralize WIV-1. Two S2X259 variants (VH=SEQ ID NO.:408, VL=SEQ ID NO.:443; VH=SEQ ID NO.:408, VL=SEQ ID NO.:445) neutralized all of the all of the pseudotyped VSV viruses with an IC50 of less than 100 ng/mL.

Three additional S2X259 variant antibodies were generated and tested for breadth of binding (ELISA), neutralization against VSV pseudoviruses in Vero E6 cells and Vero-TMPRSS2 cells, neutralization against MLV pseudoviruses in Vero E6 cells, binding to SARS-CoV-1 RBD and SARS-CoV-2 RBD (by BLI), and cell line productivity and elution profiling. These three additional S2X259 variant antibodies have VH and VL amino acid sequences as follows:
VH=SEQ ID NO.:458, VL=SEQ ID NO.:445
VH=SEQ ID NO:459, VL=SEQ ID NO.:445
VH=SEQ ID NO.:460, VL=SEQ ID NO.:445

In the ELISA binding studies, the following viruses were used: Clade 1a (SARS-CoV, WIV1); Clade 2 (SARS-CoV-2, RatG13, PangGD, PangGX); Clade 2 (Anlong112, YN2013, SC2018, SX2011, ZC45); Clade 3 (BtkY72, BGR2008); SARS-CoV-2 mutants (N501Y, Y453F, N439K, K417V, E484K, B.1.351, B.1.429, P.1, B.1.1222). Two other S2X259 variant antibodies, as well as antibodies S2H9 and S2K146, were used as controls. Binding by the three additional S2X259 variants is summarized in Table 26. These results were comparable to or better than those achieved with other S2X259 antibodies, including an antibody having the VH of SEQ ID NO.:408 and the VL of SEQ ID NO.:445.

TABLE 26

| | | | | IC50 | |
|---|---|---|---|---|---|
| VH SEQ | VL SEQ | IC50 | IC50 | 100-1000 | |
| ID NO.: | ID NO.: | 1-50 ng/mL | 50-100 ng/mL | ng/mL | No binding |
| 458 | 445 | SARS-CoV; | SARS-CoV-2 | Anlong112; | |
| | | WIV1; RatG13; | PangGD; N501Y; | ZC45 | |
| | | PangGX; YN2013; | Y453F; N439K; | | |
| | | SC2018; S2X2011; | E484K; B.1.351; | | |
| | | BtKY72; | B.1.1.222 | | |
| | | BGR2008; K417V; | | | |
| | | B.1.429; P.1 | | | |
| 459 | 445 | SARS-CoV; | SARS-CoV-2; | Anlong112 | ZC45 |
| | | WIV1; | PangGD; | | |
| | | RatG13; PangGX; | BtKY72; N501Y | | |
| | | YN2013; SC2018; | Y453F; N439K | | |
| | | SX2011; | E484K; B.1.351 | | |
| | | BGR2008; | P.1; B.1.1.222 | | |
| | | K417V; B.1.129 | | | |
| 460 | 445 | SARS-CoV; WIV1 | SARS-CoV-2 | SX2011 | Anlong112; |
| | | RatG43; PangGD | | | ZC45 |
| | | PangGX; YN2013 | | | |
| | | SC2018; BtKY72 | | | |
| | | BGR2008; N501Y | | | |
| | | Y453F; N439K | | | |
| | | K417V; E484K | | | |
| | | B.1.351; B.1.429 | | | |
| | | PP.1; B.1.1.222 | | | |

Neutralization IC50 values of antibodies using VSV pseudoviruses (in VeroE6 cells and Vero-TMPRSS2 cells) and MSV pseudoviruses (in Vero E6 cells) were as shown in Table 27. Another S2X259 variant antibody (VH of SEQ ID NO.:408; VL of SEQ ID NO.:445) was included in the analysis.

TABLE 27

| | | | IC50 ng/mL | IC50 ng/mL |
|---|---|---|---|---|
| VH SEQ | VL SEQ | IC50 (ng/mL) | (VSV-Vero- | (MLV-Vero- |
| ID NO.: | ID NO.: | (VSV-VeroE6) | TMPRSS2) | TMPRSS2) |
| 458 | 445 | 105.6 | 133.1 | approx. 354.1 |
| 459 | 445 | 105.3 | 255.3 | approx. 323.2 |
| 460 | 445 | 108.7 | 260.6 | approx. 372.9 |
| 408 | 445 | 47.57 | 96.92 | approx. 370.2 |

Affinity measurements of antibodies against SARS-CoV-1 RBD and SARS-CoV-2 RBD using BLI were as shown in Tables 28 and 29. Another S2X259 variant antibody (VH of SEQ ID NO.:408; VL of SEQ ID NO.:445) was included in the analysis.

TABLE 28

| VH SEQ | VL SEQ | SARS-CoV-1 RBD | | |
|---|---|---|---|---|
| ID NO.: | ID NO.: | KD (M) | Kon (1/Ms) | Kdis (1/s) |
| 458 | 445 | 4.547E−10 | 379700 | 0.0001727 |
| 459 | 445 | 7.917E−10 | 344700 | 0.0002729 |
| 460 | 445 | 1.588E−09 | 391300 | 0.0006212 |
| 408 | 445 | 1.763E−10 | 410600 | 0.00002078 |

TABLE 29

| VH SEQ | VL SEQ | SARS-CoV-2 RBD | | |
|---|---|---|---|---|
| ID NO.: | ID NO.: | KD (M) | Kon (1/Ms) | Kdis (1/s) |
| 458 | 445 | 8.361E−11 | 601200 | 0.00005026 |
| 459 | 445 | 1.076E−10 | 600700 | 0.00006463 |
| 460 | 445 | 2.979E−10 | 594800 | 0.0001772 |
| 408 | 445 | 3.143E−11 | 661200 | 0.00002078 |

S2X259 variant antibodies were expressed in Epi-CHO cells, and productivity and elution profiles were assessed. Results are summarized in Table 30.

TABLE 30

| VH SEQ | VL SEQ | Productivity in Expi-CHO Cells | |
|---|---|---|---|
| ID NO.: | ID NO.: | (μg/mL) | Elution profile |
| 458 | 445 | 350 | clean |
| | | (high productivity) | |
| 459 | 445 | 170 | clean |
| | | (low productivity) | |
| 460 | 445 | 300 | clean |
| | | (middle/high productivity) | |

### Example 17

### Materials and Methods

### Flow-Cytometry Based Screening for Binding to CoV S Protein Expressed on Mammalian Cells

ExpiCHO cells were transfected with S protein of SARS-CoV-2, SARS-CoV and MERS-CoV, or with an empty plasmid as a negative control. The monoclonal antibodies were then tested by flow-cytometry at 10 μg/ml for their ability to stain ExpiCHO cells expressing the S protein of 2019-nCoV, SARS-CoV, MERS-CoV or Mock cell transfectants.

Transient expression of recombinant SARS-CoV-2 protein The full-length S gene of SARS-CoV-2 strain (2019-nCoV-S) isolate BetaCoV/Wuhan-Hu-1/2019 (accession number MN908947) was codon optimized for human cell expression and cloned into the phCMV1 expression vector (Genlantis). Expi-CHO cells were transiently transfected with phCMV1-SARS-CoV-2-S, phCMV1-MERS-CoV-S(London1/2012), SARS-spike_pcDNA.3 (strain SARS) or the empty phCMV1 (Mock) using Expifectamine CHO Enhancer. Two days after transfection, cells were collected, fixed, or fixed and permeabilized with saponin for immunostaining with a panel of monoclonal antibodies reactive to SARS-CoV Receptor Binding Domain (RBD). An Alexa647-labelled secondary antibody anti-human IgG Fc was used for detection. Binding of antibodies to transfected cells was analyzed by flow-cytometry using a ZE5 Cell Analyzer (Biorard) and FlowJo software (TreeStar). Positive binding was defined by differential staining of CoV-S-transfectants versus mock-transfectants.

### Competition Experiments Using Octet (BLI, Biolayer Interferometry)

Unless otherwise indicated herein, anti-His sensors (BIOSENSOR ANTI-PENTA-HIS (HIS1K)) were used to immobilize the S1 subunit protein of SARS-CoV (Sino Biological Europe GmbH). Sensors were hydrated for 10 min with Kinetics Buffer (KB; 0.01% endotoxin-free BSA, 0.002\hat{ } Tween-20, 0.005% NaN3 in PBS). SARS-CoV S1 subunit protein was then loaded for 8 min at a concentration of 10 μg/ml in KB. Antibodies were associated for 6 min at 15 μg/ml for full length mAbs nCoV-10 and nCov-6 mAbs or 5 μg/ml for Fab nCoV-4, and in a subsequent experiment comprising nCoV-1 all at 10 μg/ml. Competing antibodies were then associated at the same concentration for additional 6 mins.

### Competition Experiments Using Octet (BLI, Biolayer Interferometry)

For ACE2 competition experiments, ACE2-His (Bio-Techne AG) was loaded for 30 minutes at 5 μg/ml in KB onto anti-HIS (HIS2) biosensors (Molecular Devices-ForteBio). SARS-CoV RBD-rabbitFc or SARS-CoV-2 RBD-mouseFc (Sino Biological Europe GmbH) at 1 μg/ml was associated for 15 minutes, after a preincubation with or without antibody (30 μg/ml, 30 minutes). Dissociation was monitored for 5 minutes.

### Affinity Determination Using Octet (BLI, Biolayer Interferometry)

For K_{D} determination of full-length antibodies, protein A biosensors (Pall ForteBio) were used to immobilize recombinant antibodies at 2.7 μg/ml for 1 minute, after a hydration step for 10 minutes with Kinetics Buffer. Association curves were recorded for 5 min by incubating the antibody-coated sensors with different concentration of SARS-CoV RBD (Sino Biological) or SARS-CoV-2 RBD (produced in house; residues 331-550 of spike from BetaCoV/Wuhan-Hu-1/2019, accession number MN908947). Highest RBD concentration tested was 10 ug/ml, then 1:2.5 serially diluted. Dissociation was recorded for 9 min by moving the sensors to wells containing KB. K_{D} values were calculated using a global fit model (Octet). Octet Red96 (ForteBio) equipment was used.

For K_{D} determination of full-length antibodies compared to Fab fragments, His-tagged RBD of SARS-CoV or SARS-CoV-2 were loaded at 3 μg/ml in KB for 15 minutes onto anti-HIS (HIS2) biosensors (Molecular Devices, ForteBio). Association of full-length antibody and Fab was performed in KB at 15 ug/ml and 5 ug/ml respectively for 5 minutes. Dissociation in KB was measured for 10 min.

### ELISA Binding

The reactivities of mAbs with SARS-CoV Spike S1 Subunit Protein (strain WH20) protein were determined by enzyme-linked immunosorbent assays (ELISA). Briefly, 96-well plates were coated with 3 μg/ml of recombinant SARS-CoV Spike S1 Subunit Protein (Sino. Biological). Wells were washed and blocked with PBS+1% BSA for 1 h at room temperature and were then incubated with serially diluted mAbs for 1 h at room temperature. Bound mAbs were detected by incubating alkaline phosphatase-conjugated goat anti-human IgG (Southern Biotechnology: 2040-04) for 1 h at room temperature and were developed by 1 mg/ml p-nitrophenylphosphate substrate in 0.1 M glycine buffer (pH 10.4) for 30 min at room temperature. The optical density (OD) values were measured at a wavelength of 405 nm in an ELISA reader (Powerwave 340/96 spectrophotometer, BioTek).

### Neutralization Assay

Unless otherwise indicated, Murine leukemia virus (MLV) pseudotyped with SARS-CoV-2 Spike protein (SARS-CoV-2pp) or SARS-CoV Spike protein (SARS-CoVpp) were used. DBT cells stably transfected with ACE2 (DBT-ACE2) were used as target cells. SARS-CoV-2pp or SARS-CoVpp was activated with trypsin TPCK at 10 ug/ml. Activated SARS-CoV-2pp or SARS-CoVpp was added to a dilution series of antibodies (starting 50 ug/ml final concentration per antibody, 3-fold dilution). DBT-ACE2 cells were added to the antibody-virus mixtures and incubated for 48h. Luminescence was measured after aspirating cell culture supernatant and adding steady-GLO substrate (Promega).

Unless otherwise indicated, pseudoparticle neutralization assays use a VSV-based luciferase reporter pseudotyping system (Kerafast). VSV pseudoparticles and antibody are mixed in DMEM and allowed to incubate for 30 minutes at 37 C. The infection mixture is then allowed to incubate with Vero E6 cells for 1 h at 37C, followed by the addition of DMEM with Pen-Strep and 10% FBS (infection mixture is not removed). The cells are incubated at 37C for 18-24 hours. Luciferase is measured using an Ensight Plate Reader (Perkin Elmer) after the addition of Bio-Glo reagent (Promega).

### SPR Single-Cycle Kinetics

SPR experiments were carried out with a Biacore T200 instrument using a single-cycle kinetics approach. S309 IgG was captured on the surface and increasing concentrations of purified SARS-CoV-2 RBD, either glycosylated or deglycosylated, were injected. Association and dissociation kinetics were monitored and fit to a binding model to determine affinity.

### Expression of Recombinant Antibodies

Recombinant antibodies were expressed in ExpiCHO cells transiently co-transfected with plasmids expressing the heavy and light chain as previously described. (Stettler et al. (2016) Specificity, cross-reactivity, and function of antibodies elicited by Zika virus infection. Science, 353(6301), 823-826) Monoclonal antibodies S303, S304, S306, S309, S310, and S315 were expressed as rIgG-LS antibodies. The LS mutation confers a longer half-life in vivo. (Zalevsky et al. (2010) Enhanced antibody half-life improves in vivo activity. Nature Biotechnology, 28(2), 157-159)

### Sequence Alignment

SARS-CoV-2 genomics sequences were downloaded from GISAID on March 29th 2020, using the “complete (>29,000 bp)” and “low coverage exclusion” filters. Bat and pangolin sequences were removed to yield human-only sequences. The spike ORF was localized by performing reference protein (YP_009724390.1)-genome alignments with GeneWise2. Incomplete matches and indel-containing ORFs were rescued and included in downstream analysis. Nucleotide sequences were translated in silico using seqkit. Sequences with more than 10% undetermined aminoacids (due to N basecalls) were removed. Multiple sequence alignment was performed using MAFFT. Variants were determined by comparison of aligned sequences (n=2,229) to the reference sequence using the R/Bioconductor package Biostrings. A similar strategy was used to extract and translate spike protein sequences from SARS-CoV genomes sourced from ViPR (search criteria: SARS-related coronavirus, full-length genomes, human host, deposited before December 2019 to exclude SARS-CoV-2, n=53). Sourced SARS-CoV genome sequences comprised all the major published strains, such as Urbani, Tor2, TW1, P2, Frankfurt1, among others. Pangolin sequences as shown by Tsan-Yuk Lam et al were sourced from GISAID. Bat sequences from the three clades of Sarbecoviruses as shown by Lu et al (Lancet 2020) were sourced from Genbank. Civet and racoon dog sequences were similarly sourced from Genbank.

Generation of Stable Overexpression Cell Lines

Lentiviruses were generated by co-transfection of Lenti-X 293T cells (Takara) with lentiviral expression plasmids encoding DC-SIGN (CD209), L-SIGN (CLEC4M), SIGLEC1, TMPRSS2 or ACE2 (all obtained from Genecopoeia) and the respective lentiviral helper plasmids. Forty-eight hours post transfection, lentivirus in the supernatant was harvested and concentrated by ultracentrifugation for 2 h at 20,000 rpm. Lenti-X 293T (Takara), Vero E6 (ATCC), MRC5 (Sigma-Aldrich), A549 (ATCC) were transduced in the presence of 6 ug/mL polybrene (Millipore) for 24 h. Cell lines overexpressing two transgenes were transduced subsequently. Selection with puromycin and/or blasticidin (Gibco) was started two days after transduction and selection reagent was kept in the growth medium for all subsequent culturing. Single cell clones were derived from the A549-ACE2-TMPRSS2 cell line, all other cell lines represent cell pools.

SARS-CoV-2 Neutralization

Vero E6 or Vero E6-TMPRSS2 cells cultured in DMEM supplemented with 10% FBS (VWR) and 1× Penicillin/Streptomycin (Thermo Fisher Scientific) were seeded in black 96-well plates at 20,000 cells/well. Serial 1:4 dilutions of the monoclonal antibodies were incubated with 200 pfu of SARS-CoV-2 (isolate USA-WA1/2020, passage 3, passaged in Vero E6 cells) for 30 min at 37° C. in a BSL-3 facility. Cell supernatant was removed and the virus-antibody mixture was added to the cells. 24 h post infection, cells were fixed with 4% paraformaldehyde for 30 min, followed by two PBS (pH 7.4) washes and permeabilization with 0.25% Triton X-100 in PBS for 30 min. After blocking in 5% milk powder/PBS for 30 min, cells were incubated with a primary antibody targeting SARS-CoV-2 nucleocapsid protein (Sino Biological, cat. 40143-R001) at a 1:2000 dilution for 1 h. After washing and incubation with a secondary Alexa647-labeled antibody mixed with 1 ug/ml Hoechst33342 for 1 hour, plates were imaged on an automated cell-imaging reader (Cytation 5, Biotek) and nucleocapsid-positive cells were counted using the manufacturer's supplied software.

SARS-CoV-2-Nluc Neutralization

Neutralization was determined using SARS-CoV-2-Nluc, an infectious clone of SARS-CoV-2 (based on strain 2019-nCoV/USA_WA1/2020) encoding nanoluciferase in place of the viral ORF7, which demonstrates comparable growth kinetics to wild type virus (Xie et al., Nat Comm, 2020, https://doi.org/10.1038/s41467-020-19055-7). Cells were seeded into black-walled, clear-bottom 96-well plates at 20,000 cells/well (293T cells were seeded into poly-L-lysine-coated wells at 35,000 cells/well) and cultured overnight at 37° C. The next day, 9-point 4-fold serial dilutions of antibodies were prepared in infection media (DMEM+10% FBS). SARS-CoV-2-Nluc was diluted in infection media at the indicated MOI, added to the antibody dilutions and incubated for 30 min at 37° C. Media was removed from the cells, mAb-virus complexes were added, and cells were incubated at 37° C. for 24 h. Media was removed from the cells, Nano-Glo luciferase substrate (Promega) was added according to the manufacturer's recommendations, incubated for 10 min at RT and luciferase signal was quantified on a VICTOR Nivo plate reader (Perkin Elmer).

SARS-CoV-2 Pseudotyped VSV Production and Neutralization

To generate SARS-CoV-2 pseudotyped vesicular stomatitis virus, Lenti-X 293T cells (Takara) were seeded in 10-cm dishes for 80% next day confluency. The next day, cells were transfected with a plasmid encoding for SARS-CoV-2 S-glycoprotein (YP_009724390.1) harboring a C-terminal 19 aa truncation using TransIT-Lenti (Mirus Bio) according to the manufacturer's instructions. One day post-transfection, cells were infected with VSV(G*ΔG-luciferase) (Kerafast) at an MOI of 3 infectious units/cell. Viral inoculum was washed off after one hour and cells were incubated for another day at 37° C. The cell supernatant containing SARS-CoV-2 pseudotyped VSV was collected at day 2 post-transfection, centrifuged at 1000×g for 5 minutes to remove cellular debris, aliquoted, and frozen at −80° C.

For viral neutralization, Cells were seeded into black-walled, clear-bottom 96-well plates at 20,000 cells/well (293T cells were seeded into poly-L-lysine-coated wells at 35,000 cells/well) and cultured overnight at 37° C. The next day, 9-point 4-fold serial dilutions of antibodies were prepared in media. SARS-CoV-2 pseudotyped VSV was diluted 1:30 in media in the presence of 100 ng/mL anti-VSV-G antibody (clone 8G5F11, Absolute Antibody) and added 1:1 to each antibody dilution. Virus:antibody mixtures were incubated for 1 hour at 37° C. Media was removed from the cells and 50 L of virus:antibody mixtures were added to the cells. One hour post-infection, 100 μL of media was added to all wells and incubated for 17-20 hours at 37° C. Media was removed and 50 μL of Bio-Glo reagent (Promega) was added to each well. The plate was shaken on a plate shaker at 300 RPM at room temperature for 15 minutes and RLUs were read on an EnSight plate reader (Perkin-Elmer).

Transfection-Based Attachment Receptor Screen

Lenti-X 293T cells (Takara) were transfected with plasmids encoding the following receptor candidates (all purchased from Genecopoeia): ACE2 (NM_021804), DC-SIGN (NM_021155), L-SIGN (BC110614), LGALS3 (NM_002306), SIGLEC1 (NM_023068), SIGLEC3 (XM_057602), SIGLEC9 (BC035365), SIGLEC10 (NM_033130), MGL (NM_182906), MINCLE (NM_014358), CD147 (NM_198589), ASGR1 (NM 001671.4), ASGR2 (NM_080913), NRP1 (NM_003873). One day post transfection, cells were infected with SARS-CoV-2 pseudotyped VSV at 1:20 dilution in the presence of 100 ng/mL anti-VSV-G antibody (clone 8G5F11, Absolute Antibody) at 37° C. One hour post-infection, 100 μL of media was added to all wells and incubated for 17-20 hours at 37° C. Media was removed and 50 μL of Bio-Glo reagent (Promega) was added to each well. The plate was shaken on a plate shaker at 300 RPM at room temperature for 15 minutes and RLUs were read on an EnSight plate reader (Perkin-Elmer).

Trans-Infection

Parental HeLa cells or HeLa cells stably expressing DC-SIGN, L-SIGN or SIGLEC1 were seeded at 5,000 cells per well in black-walled clear-bottom 96-well plates. One day later, cells reached about 50% confluency and were inoculated with SARS-CoV-2 pseudotyped VSV at 1:10 dilution in the presence of 100 ng/mL anti-VSV-G antibody (clone 8G5F11, Absolute Antibody) at 37° C. for 2 h. For antibody-mediated inhibition of trans-infection, cells were pre-incubated with 10 ug/mL anti-SIGLEC1 antibody (Biolegend, clone 7-239) for 30 min. After 2 h inoculation, cells were washed four times with complete medium and 10,000 VeroE6-TMPRSS2 cells per well were added and incubated 17-20 h at 37° C. for trans-infection. Media was removed and 50 μL of Bio-Glo reagent (Promega) was added to each well. The plate was shaken on a plate shaker at 300 RPM at room temperature for 15 minutes and RLUs were read on an EnSight plate reader (Perkin-Elmer).

Cell-Cell Fusion of CHO-S Cells

CHO cells stably expressing SARS-CoV-2 S-glycoprotein were seeded in 96 well plates for microscopy (Thermo Fisher Scientific) at 12′500 cells/well and the following day, different concentrations of mAbs and nuclei marker Hoechst (final dilution 1:1000) were added to the cells and incubated for additional 24h hours. Fusion degree was established using the Cytation 5 Imager (BioTek) and an object detection protocol was used to detect nuclei as objects and measure their size. The nuclei of fused cells (i.e., syncytia) are found aggregated at the center of the syncitia and are recognized as a unique large object that is gated according to its size. The area of the objects in fused cells divided by the total area of all the object multiplied by 100 provides the percentage of fused cells

Immunofluorescence Analysis

HEK 293T cells were seeded onto poly-D-Lysine-coated 96-well plates (Sigma-Aldrich) and fixed 24 h after seeding with 4% paraformaldehyde for 30 min, followed by two PBS (pH 7.4) washes and permeabilization with 0.25% Triton X-100 in PBS for 30 min. Cells were incubated with primary antibodies anti-DC-SIGN/L-SIGN (Biolegend, cat. 845002, 1:500 dilution), anti-DC-SIGN (Cell Signaling, cat. 13193S, 1:500 dilution), anti-SIGLEC1 (Biolegend, cat. 346002, 1:500 dilution) or anti-ACE2 (R&D Systems, cat. AF933, 1:200 dilution) diluted in 3% milk powder/PBS for 2 h at room temperature. After washing and incubation with a secondary Alexa647-labeled antibody mixed with 1 ug/ml Hoechst33342 for 1 hour, plates were imaged on an inverted fluorescence microscope (Echo Revolve).

ACE2/TMPRSS2 RT-qPCR

RNA was extracted from the cells using the NucleoSpin RNA Plus kit (Macherey-Nagel) according to the manufacturer's protocol. RNA was reverse transcribed using the High Capacity cDNA Reverse Transcription kit (Applied Biosystems) according to the manufacturer's instructions. Intracellular levels of ACE2 (Forward Primer: CAAGAGCAAACGGTTGAACAC (SEQ ID NO.:461), Reverse Primer: CCAGAGCCTCTCATTGTAGTCT (SEQ ID NO.:462)), HPRT (Forward Primer: CCTGGCGTCGTGATTAGTG (SEQ ID NO.:463), Reverse Primer: ACACCCTTTCCAAATCCTCAG (SEQ ID NO.:464)), and TMPRSS2 (Forward Primer: CAAGTGCTCCRACTCTGGGAT (SEQ ID NO.:465), Reverse Primer: AACACACCGRTTCTCGTCCTC (SEQ ID NO.:466)) were quantified using the Luna Universal qPCR Master Mix (New England Biolabs) according to the manufacturer's protocol. Levels of ACE2 and TMPRSS2 were normalized to HPRT. Hela cells were used as the reference sample. All qPCRs were run on a QuantStudio 3 Real-Time PCR System (Applied Biosystems).

SARS2 D614G Spike Production and Biotinylation

Prefusion-stabilized SARS2 D614G spike (comprising amino acid sequence Q14 to K1211) with a C-terminal TEV cleavage site, T4 bacteriophage fibritin foldon, 8× His-, Avi- and EPEA-tag was transfected into HEK293 Freestyle cells, using 293fectin as a transfection reagent. Cells were left to produce protein for three days at 37° C. Afterwards, supernatant was harvested by centrifuging cells for 30 minutes at 500×g, followed by another spin for 30 minutes at 4000×g. Cell culture supernatant was filtered through a 0.2 um filter and loaded onto a 5 mL C-tag affinity matrix column, pre-equilibrated with 50 mM Tris pH 8 and 200 mM NaCl. SARS2 D614G spike was eluted, using 10 column volumes of 100 mM Tris, 200 mM NaCl and 3.8 mM SEPEA peptide. Elution peak was concentrated and injected on a Superose 6 increase 10/300 GL gel filtration column, using 50 mM Tris pH 8 and 200 mM NaCl as a running buffer. SEC fractions corresponding to monodisperse SARS2 D614G spike were collected and flash frozen in liquid nitrogen for storage at −80° C. Purified SARS2 D614G spike protein was biotinylated using BirA500 biotinylation kit from Avidity. To 50 ug of spike protein, 5 ug of BirA, and 11 uL of BiomixA and BiomixB was added. Final spike protein concentration during the biotinylation reaction was ˜1 uM. The reaction was left to proceed for 16 hours at 4° C. Then, protein was desalted using two Zeba spin columns pre-equilibrated with 1×PBS pH 7.4.

Flow Cytometry Analysis for DC-SIGN, L-SIGN, SIGLEC1 and ACE-2

HEK 293T cells expressing DC-SIGN, L-SIGN, SIGLEC1 or ACE2 were resuspended at 4×10⁶ cells/mL and 100 μL per well were seeded onto V-bottom 96-well plates (Corning, 3894). The plate was centrifuged at 2,000 rpm for 5 minutes and washed with PBS (pH 7.4). The cells were resuspended in 200 μL of PBS containing Ghost violet 510 viability dye (Cell Signaling, cat. 13-0870-T100, 1:1,000 dilution), incubated for 15 minutes on ice and then washed. The cells were resuspended in 100 μL of FACS buffer prepared with 0.5% BSA (Sigma-Aldrich) in PBS containing the primary antibodies at a 1:100 dilution: mouse anti-DC/L-SIGN (Biolegend, cat. 845002), rabbit anti-DC-SIGN (Cell Signaling, cat. 13193), mouse anti-SIGLEC1 (Biologend, cat. 346002) or goat anti-ACE2 (R&D Systems, cat. AF933). After 1 h incubation on ice, the cells were washed two times and resuspended in FACS buffer containing the Alexa Fluor-488-labeled secondary antibodies at a 1:200 dilution: goat anti-mouse (Invitrogen cat. A11001), goat anti-rabbit (Invitrogen cat. A11008) or donkey anti-goat (Invitrogen cat. A11055). After incubation for 45 min on ice, the cells were washed three times with 200 μL of FACS buffer and fixed with 200 μL of 4% PFA (Alfa Aesar) for 15 mins at room temperature. Cells were washed three times, resuspended in 200 μL of FACS buffer and analyzed by flow cytometry using the CytoFLEX flow cytometer (Beckman Coulter).

Flow cytometry of SARS-CoV-2 Spike and RBD binding to cells Biotinylated SARS-CoV-2 Spike D614G protein (Spikebiotin, in-house generated) or the biotinylated SARS-CoV-2 Spike receptor-binding domain (RBDbiotin, Sino Biological, 40592-V08B) were incubated with Alexa Fluor® 647 streptavidin (AF647-strep, Invitrogen, S21374) at a 1:20 ratio by volume for 20 min at room temperature. The labeled proteins were then stored at 4° C. until further use. Cells were dissociated with TrpLE Express (Gibco, 12605-010) and 10⁵ cells were transferred to each well of a 96-well V bottom plate (Corning, 3894). Cells were washed twice in flow cytometry buffer (2% FBS in PBS (w/o Ca/Mg)) and stained with Spikebiotin-AF647-strep at a final concentration of 20 μg/ml or RBDbiotin-AF647-strep at a final concentration of 7.5 μg/ml for 1 h on ice. Stained cells were washed twice with flow cytometry buffer, resuspended in 1% PFA (Electron Microscopy Sciences, 15714-S) and analyzed with the Cytoflex LX (Beckman Coulter).

Recombinant Expression of SARS-CoV-2-Specific mAbs.

Human mAbs were isolated from plasma cells or memory B cells of SARS-CoV-2 immune donors, as previously described. Recombinant antibodies were expressed in ExpiCHO cells at 37° C. and 8% C02. Cells were transfected using ExpiFectamine. Transfected cells were supplemented 1 day after transfection with ExpiCHO Feed and ExpiFectamine CHO Enhancer. Cell culture supernatant was collected eight days after transfection and filtered through a 0.2 μm filter. Recombinant antibodies were affinity purified on an AKTA xpress FPLC device using 5 mL HiTrap™ MabSelect™ PrismA columns followed by buffer exchange to Histidine buffer (20 mM Histidine, 8% sucrose, pH 6) using HiPrep 26/10 desalting columns

SARS-CoV-2 Infection Model in Hamster

Virus Preparation

The SARS-CoV-2 strain used in this study, BetaCov/Belgium/GHB-03021/2020 (EPI ISL 109 407976|2020-02-03), was recovered from a nasopharyngeal swab taken from an RT-qPCR confirmed asymptomatic patient who returned from Wuhan, China in February 2020. A close relation with the prototypic Wuhan-Hu-1 2019-nCoV (GenBank accession 112 number MN908947.3) strain was confirmed by phylogenetic analysis. Infectious virus was isolated by serial passaging on HuH7 and Vero E6 cells; passage 6 virus was used for the study described here. The titer of the virus stock was determined by end-point dilution on Vero E6 cells by the Reed and Muench method.

Cells

Vero E6 cells (African green monkey kidney, ATCC CRL-1586) were cultured in minimal essential medium (Gibco) supplemented with 10% fetal bovine serum (Integro), 1% L-glutamine (Gibco) and 1% bicarbonate (Gibco). End-point titrations were performed with medium containing 2% fetal bovine serum instead of 10%.

SARS-CoV-2 Infection Model in Hamsters

The hamster infection model of SARS-CoV-2 has been described before. The specific study design is shown in the schematic below. In brief, wild-type Syrian Golden hamsters (Mesocricetus auratus) were purchased from Janvier Laboratories and were housed per two in ventilated isolator cages (IsoCage N Biocontainment System, Tecniplast) with ad libitum access to food and water and cage enrichment (wood block). The animals were acclimated for 4 days prior to study start. Housing conditions and experimental procedures were approved by the ethics committee of animal experimentation of KU Leuven (license P065-2020). Female 6-8 week old hamsters were anesthetized with ketamine/xylazine/atropine and inoculated intranasally with 50 μL containing 2×10⁶ TCID50 SARS-CoV-2 (day 0).

Treatment Regimen

Animals were prophylactically treated 48h before infection by intraperitoneal administration (i.p.) and monitored for appearance, behavior, and weight. At day 4 post infection (p.i.), hamsters were euthanized by i.p. injection of 500 μL Dolethal (200 mg/mL sodium pentobarbital, Vétoquinol SA). Lungs were collected and viral RNA and infectious virus were quantified by RT-qPCR and end-point virus titration, respectively. Blood samples were collected before infection for PK analysis.

SARS-CoV-2 RT-qPCR

Collected lung tissues were homogenized using bead disruption (Precellys) in 350 μL RLT buffer (RNeasyMinikit, Qiagen) and centrifuged (10.000 rpm, 5 min) to pellet the cell debris. RNA was extracted according to the manufacturer's instructions. Of 50 μL eluate, 4 μL was used as a template in RT-qPCR reactions. RT-qPCR was performed on a LightCycler96 platform (Roche) using the iTaq Universal Probes One-Step RT-qPCR kit (BioRad) with N2 primers and probes targeting the nucleocapsid. Standards of SARS-CoV-2 cDNA (IDT) were used to express viral genome copies per mg tissue or per mL serum.

End-Point Virus Titrations

Lung tissues were homogenized using bead disruption (Precellys) in 350 μL minimal essential medium and centrifuged (10,000 rpm, 5 min, 4° C.) to pellet the cell debris. To quantify infectious SARS-CoV-2 particles, endpoint titrations were performed on confluent Vero E6 cells in 96-well plates. Viral titers were calculated by the Reed and Muench method using the Lindenbach calculator and were expressed as 50% tissue culture infectious dose (TCID50) per mg tissue.

Histology

For histological examination, the lungs were fixed overnight in 4% formaldehyde and embedded in paraffin. Tissue sections (5 m) were analyzed after staining with hematoxylin and eosin and scored blindly for lung damage by an expert pathologist. The scored parameters, to which a cumulative score of 1 to 3 was attributed, were the following: congestion, intra-alveolar hemorrhagic, apoptotic bodies in bronchus wall, necrotizing bronchiolitis, perivascular edema, bronchopneumonia, perivascular inflammation, peribronchial inflammation and vasculitis.

Binding of Immunocomplexes to Hamster Monocytes

Immunocomplexes (IC) were generated by complexing S309 mAb (hamster IgG, either wt or N297A) with a biotinylated anti-idiotype fab fragment and Alexa-488-streptavidin, using a precise molar ratio (4:8:1, respectively). Pre-generated fluorescent IC were serially diluted incubated at 4° C. for 3 hrs with freshly revitalized hamster splenocytes, obtained from a naïve animal. Cellular binding was then evaluated by cytometry upon exclusion of dead cells and physical gating on monocyte population. Results are expressed as Alexa-488 mean florescent intensity of the entire monocyte population.

Bioinformatic Analyses

Processed Human Lung Cell Atlas (HLCA) data and cell-type annotations were downloaded from Github (github.com/krasnowlab/HiLCA). Processed single-cell transcriptome data and annotation of lung epithelial and immune cells from SARS-CoV-2 infected individuals were downloaded from NCBI GEO database (ID: GSE158055) and Github (github.com/zhangzlab/covid_balf). Available sequence data from the second single-cell transcriptomics study by Liao et al. were downloaded from NCBI SRA (ID: PRJNA608742) for inspection of reads corresponding to viral RNA. The proportion of sgRNA relative to genomic RNA was estimated by counting TRS-containing reads supporting a leader-TRS junction. Criteria and methods for detection of leader-TRS junction reads were adapted from Alexandersen et al. The viral genome reference and TRS annotation was based on Wuhan-Hu-1 NC_045512.2/MN908947. Only 2 samples from individuals with severe COVID-19 had detectable leader-TRS junction reads (SRR11181958, SRR11181959).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 63/010,025, filed Apr. 14, 2020, U.S. Patent Application No. 63/015,399, filed Apr. 24, 2020, U.S. Patent Application No. 63/019,926, filed May 4, 2020, U.S. Patent Application No. 63/021,063, filed May 6, 2020, U.S. Patent Application No. 63/023,808, filed May 12, 2020, U.S. Patent Application No. 63/030,254, filed May 26, 2020, U.S. Patent Application No. 63/036,631, filed Jun. 9, 2020, U.S. Patent Application No. 63/046,452, filed Jun. 30, 2020, U.S. Patent Application No. 63/057,557, filed Jul. 28, 2020, U.S. Patent Application No. 63/091,841, filed Oct. 14, 2020, U.S. Patent Application No. 63/166,879, filed Mar. 26, 2021, U.S. Patent Application No. 63/170,360, filed Apr. 2, 2021, and U.S. Patent Application No. 63/171,892, filed Apr. 7, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein:
(i) the CDRH1 comprises or consists of the amino acid sequence according to any one of SEQ ID NOs.: 409, 23, 33, 38, 46, 53, 55, 63, 70, 72, 83, 93, 103, 113, 123, 137, 147, 160, 166, 181, 191, 201, 211, 221, 233, 243, 268, 305, 315, 325, 330, 335, 349, 359, 369, 379, 389, 399, 419, or 449, or a sequence variant thereof comprising one, two, or three amino acid substitutions, one or more of which substitutions is optionally a conservative substitution and/or is a substitution to a germline-encoded amino acid;
(ii) the CDRH2 comprises or consists of the amino acid sequence according to any one of SEQ ID NOs.: 410, 24, 31, 36, 39, 48, 51, 56, 65, 67, 73, 83, 93, 103, 113, 123, 137, 147, 161, 167, 182, 192, 202, 212, 222, 234, 244, 263, 269, 285, 287, 289, 293, 299, 301, 306, 316, 326, 331, 336, 350, 360, 370, 380, 390, 400, 420, 447, 457, or a sequence variant thereof comprising one, two, or three amino acid substitutions, one or more of which substitutions is optionally a conservative substitution and/or is a substitution to a germline-encoded amino acid;
(iii) the CDRH3 comprises or consists of the amino acid sequence according to any one of SEQ ID NOs.: 411, 25, 40, 57, 74, 84, 94, 104, 114, 124, 138, 148, 156, 162, 168, 183, 193, 203, 213, 223, 235, 245, 254, 257, 259, 261, 265, 271, 273, 275, 277, 279, 281, 290, 294, 296, 307, 317, 324, 327, 332, 337, 351, 361, 371, 381, 391, 401, 421, or 435, or a sequence variant thereof comprising one, two, or three amino acid substitutions, one or more of which substitutions is optionally a conservative substitution and/or is a substitution to a germline-encoded amino acid;
(iv) the CDRL1 comprises or consists of the amino acid sequence according to any one of SEQ ID NOs.: 413, 27, 42, 59, 76, 86, 96, 106, 116, 126, 140, 150, 163, 169, 185, 195, 205, 215, 225, 237, 247, 309, 319, 328, 333, 338, 353, 363, 373, 383, 393, 403, or 423, or a sequence variant thereof comprising one, two, or three amino acid substitutions, one or more of which substitutions is optionally a conservative substitution and/or is a substitution to a germline-encoded amino acid;
(v) the CDRL2 comprises or consists of the amino acid sequence according to any one of SEQ ID NOs.: 414, 28, 43, 60, 77, 87, 97, 107, 117, 127, 141, 151, 164, 170, 186, 196, 206, 216, 226, 238, 248, 310, 320, 329, 334, 339, 354, 364, 374, 384, 394, 404, 424, or 440, or a sequence variant thereof comprising one, two, or three amino acid substitutions, one or more of which substitutions is optionally a conservative substitution and/or is a substitution to a germline-encoded amino acid; and/or
(vi) the CDRL3 comprises or consists of the amino acid sequence according to any one of SEQ ID NOs.: 415, 29, 44, 61, 78, 88, 98, 108, 118, 128, 142, 152, 165, 171, 187, 197, 207, 217, 227, 239, 249, 283, 303, 311, 321, 355, 365, 375, 385, 395, 405, or 425, or a sequence variant thereof comprising having one, two, or three amino acid substitutions, one or more of which substitutions is optionally a conservative substitution and/or is a substitution to a germline-encoded amino acid,
wherein the antibody or antigen binding fragment is capable of binding to a SARS-CoV-2 surface glycoprotein (S) expressed on a cell surface of a host cell, on a SARS-CoV-2 virion, or both.

2. The antibody or antigen-binding fragment of claim 1, which is capable of neutralizing a SARS-CoV-2 infection in an in vitro model of infection and/or in an in vivo animal model of infection and/or in a human, wherein, optionally, the SARS-CoV-2 infection comprises a SARS-CoV-2 comprising the amino acid sequence according to SEQ ID NO.:3.

3. The antibody or antigen-binding fragment of claim 1, which is
(i) capable of binding to the surface glycoprotein of two or more (e.g., two, three, four, five, or more) sarbecoviruses expressed on a cell surface of a host cell, on a sarbecovirus virion, or both; and/or
(ii) capable of neutralizing an infection by two or more sarbecoviruses in an in vitro model of infection and/or in an in vivo animal model of infection and/or in a human.

4. The antibody or antigen-binding fragment of claim 1, comprising CDRH1, CDRH2, CDRH3, CDRL1, CDRL2, and CDRL3 amino acid sequences according to SEQ ID NOs.:
(i) 409-411 and 413-415, respectively;
(ii) 23 or 160, 31, 25 or 162, and 27-29 or 163-165, respectively;
(iii) 33, 24 or 161, 25 or 162, and 27-29 or 163-165, respectively;
(iv) 33, 31, 25 or 162, and 27-29 or 163-165, respectively;
(v) 33, 36, 25 or 162, and 27-29 or 163-165, respectively;
(vi) 38-40 and 42-44, respectively;
(vii) 46, 39 or 167, 40 or 168, and 42-44 or 169-171, respectively;
(viii) 38 or 166, 48, 40 or 168 and 42-44 or 169-171, respectively;
(ix) 46, 48, 40 or 168 and 42-44 or 169-171, respectively;
(x) 46, 51, 40 or 168, and 42-44 or 169-171, respectively;
(xi) 53, 48, 40 or 168, and 42-44 or 169-171, respectively;
(xii) 55-57 and 59-61, respectively;
(xiii) 63, 56, 57 and 59-61, respectively;
(xiv) 55, 65, 57 and 59-61, respectively;
(xv) 63, 67, 57, and 59-61, respectively;
(xvi) 63, 65, 57 and 59-61, respectively;
(xvii) 70, 65, 57, and 59-61, respectively;
(xviii) 72-74 and 76-78, respectively;
(xix) 82-84 and 86-88, respectively;
(xx) 92-94 and 96-98, respectively;
(xxi) 102-104, and 106-108, respectively;
(xxii) 112-114 and 116-118, respectively;
(xxiii) 122-124 and 126-128, respectively;
(xxiv) 136-138 and 140-142, respectively;
(xxv) 146-148 and 150-152, respectively;
(xxvi) 112, 113, 156 and 116-118, respectively
(xxvii) 181-183 and 185-187, respectively;
(xxviii) 191-193 and 195-197, respectively;
(xxix) 201-203 and 205-207, respectively;
(xxx) 211-213 and 215-217, respectively;
(xxxi) 221-223 and 225-227, respectively;
(xxxii) 233-235 and 237-239, respectively;
(xxxiii) 243-245 and 247-249 respectively;
(xxxiv) 211, 212, any one of 254, 257, 259, 261, or 324 and 215-217, respectively;
(xxxv) any one of 221, 268, or 325, any one of 222, 263, 269, or 326, any one of 223, 265, 271, 273, or 327 and 225, 226 or 328, and 227 or 329, respectively;
(xxxvi) 233 or 330, 234 or 331, any one of 235, 275, 277, 279, 281, or 332, and any one of 237, 282 or 333, 238 or 234, and 239, respectively;
(xxxvii) 243 or 335, any one of 244, 285, 287, 289, 293, 299, 301, or 336, any one of 245, 290, 294, 296, or 337, and 247 or 338, 248 or 339, and 249 or 303, respectively;
(xxxviii) 305-307 and 309-311, respectively;
(xxxix) 315-317 and 319-321, respectively;
(xxxx) 349-351 and 353-355, respectively;
(xxxxi) 359-361 and 363-365, respectively;
(xxxxii) 369-371 and 373-375, respectively;
(xxxxiii) 379-381 and 383-385, respectively;
(xxxxiv) 389-391 and 393-395, respectively;
(xxxxv) 399, 400, 401 or 435, and 403, 404 or 440, and 405, respectively;
(xxxxvi) 23-25 and 27-29, respectively;
(xxxxvii) 419-421 and 423-425, respectively;
(xxxxviii) 409, 447, 411, and 413-415, respectively;
(xxxxix) 449, 410, 411, and 413-415, respectively;
(xxxxx) 449, 447, 411, and 413-415, respectively;
(xxxxxi) 409, 457, 411, and 413-415, respectively;
(xxxxxii) 449, 457, 411, and 413-415, respectively.

5. An antibody, or an antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, the CDRH2, the CDRH3, the CDRL1, the CDRL2, and the CDRL3 comprise or consist of the amino acid sequences set forth in:
(i) SEQ ID NOs.: 409, 410, 411, 413, 414, and 415, respectively;
(ii) SEQ ID NOs.: 409, 447, 411, 413, 414, and 415, respectively;
(iii) SEQ ID NOs.: 409, 457, 411, 413, 414, and 415, respectively;
(iv) SEQ ID NOs.: 449, 410, 411, 413, 414, and 415, respectively;
(v) SEQ ID NOs.: 449, 447, 411, 413, 414, and 415, respectively; or
(vi) SEQ ID NOs.: 449, 457, 411, 413, 414, and 415, respectively,
and wherein the antibody or antigen-binding fragment is capable of binding to a SARS-CoV-2 surface glycoprotein (S).

6.-11. (canceled)

12. An antibody, or an antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, the CDRH2, the CDRH3, the CDRL1, the CDRL2, and the CDRL3 comprise or consist of the amino acid sequences set forth in:
(i) SEQ ID NOs.: 399, 400, 401, 403, 404, and 405, respectively;
(ii) SEQ ID NOs.: 399, 400, 435, 403, 404, and 405, respectively;
(iii) SEQ ID NOs.: 399, 400, 401, 403, 440, and 405, respectively; or
(iv) SEQ ID NOs.: 399, 400, 435, 403, 440, and 405, respectively,
and wherein the antibody or antigen-binding fragment is capable of binding to a SARS-CoV-2 surface glycoprotein (S).

13-16. (canceled)

17. The antibody or antigen-binding fragment of claim 1, wherein:
(i) the VH comprises or consists of an amino acid sequence having at least 85% identity to the amino acid sequence according to any one of SEQ ID NOs.: 22, 30, 32, 34, 35, 37, 45, 47, 49, 50, 52, 54, 62, 64, 66, 68, 69, 71, 81, 91, 101, 111, 121, 135, 145, 155, 158, 180, 190, 200, 210, 220, 232, 242, 252, 253, 255, 256, 258, 260, 262, 264, 266, 267, 270, 272, 274, 276, 278, 280, 284, 286, 288, 291, 292, 295, 297, 298, 300, 304, 314, 348, 358, 368, 378, 388, 398, 408, 418, 428, 432, 434, 437, 446, 448, 448, 459, and 460, wherein the variation is optionally limited to one or more framework regions and/or the variation comprises one or more substitution to a germline-encoded amino acid; and/or
(ii) the VL comprises or consists of an amino acid sequence having at least 85% identity to the amino acid sequence according to any one of SEQ ID NOs.: 26, 41, 58, 75, 85, 95, 105, 115, 125, 139, 149, 184, 194, 204, 214, 224, 230, 236, 246, 282, 302, 308, 319, 352, 362, 372, 382, 392, 402, 412, 422, 439, 442, 443, 444, and 445, wherein the variation is optionally limited to one or more framework regions and/or the variation comprises one or more substitution to a germline-encoded amino acid.

18. The antibody or antigen-binding fragment of claim 1, wherein the VH comprises or consists of any VH amino acid sequence set forth in Table 3, and wherein the VL comprises or consists of any VL amino acid sequence set forth in Table 3, wherein, optionally, the VH and the VL comprise or consist of the amino acid sequences according to SEQ ID NOs.:
(i) any one of 408, 446, 448, 458, 459, and 460 and any one of 412, 442, 443, 444, and 445, respectively, optionally according to (a) 408 and 412, respectively; (b) 408 and 442, respectively; (c) 408 and 443, respectively; (d) 408 and 444, respectively; (e) 408 and 445, respectively; (f) 428 and 412, respectively; (g) 428 and 442, respectively; (h) 428 and 443, respectively; (i) 428 and 444, respectively; (j) 428 and 445, respectively; (k) 446 and 412, respectively; (l) 446 and 442, respectively; (m) 446 and 443, respectively; (n) 446 and 444, respectively; (o) 446 and 445, respectively; (p) 448 and 412, respectively; (q) 448 and 442, respectively; (r) 448 and 443, respectively; (s) 448 and 444, respectively; (t) 448 and 445, respectively; (u) 458 and 412, respectively; (v) 458 and 442, respectively; (w) 458 and 443, respectively; (x) 458 and 444, respectively; (y) 458 and 445, respectively; (z) 459 and 412, respectively; (aa) 459 and 442, respectively; (bb) 459 and 443, respectively; (cc) 459 and 444, respectively; (dd) 459 and 445, respectively; (ee) 460 and 412, respectively; (ff) 460 and 442, respectively; (gg) 460 and 443, respectively; (hh) 460 and 444, respectively; or (ii) 460 and 445, respectively;
(ii) 30 and 26, respectively;
(iii) 32 and 26, respectively;
(iv) 34 and 26, respectively;
(v) 35 and 26, respectively;
(vi) 37 and 41, respectively;
(vii) 45 and 41, respectively;
(viii) 47 and 41, respectively;
(ix) 49 and 41, respectively;
(x) 50 and 41, respectively;
(xi) 52 and 41, respectively;
(xii) 54 and 58, respectively;
(xiii) 62 and 58, respectively;
(xiv) 64 and 58, respectively;
(xv) 66 and 58, respectively;
(xvi) 68 and 58, respectively;
(xvii) 69 and 58, respectively;
(xviii) 71 and 75, respectively;
(xix) 81 and 85, respectively;
(xx) 91 and 95, respectively;
(xxi) 101 or 158 and 105, respectively;
(xxii) 111 or 155 and 115, respectively;
(xxiii) 121 and 125, respectively;
(xxiv) 135 and 139, respectively;
(xxv) 145 and 149, respectively;
(xxvi) 180 and 184, respectively;
(xxvii) 190 and 194, respectively;
(xxviii) 200 and 204, respectively;
(xxix) 210 and 214, respectively;
(xxx) 220 and 224, respectively;
(xxxi) 220 and 230, respectively;
(xxxii) 232 and 236, respectively;
(xxxiii) 242 and 246, respectively;
(xxxiv) any one of 252, 253, 255, 256, 258, or 260 and 214, respectively;
(xxxv) any one of 262, 264, 266, 267, 270, or 272 and 224, respectively;
(xxxvi) any one of 274, 276, 278, or 280 and 236 or 282, respectively
(xxxvii) any one of 284, 286, 288, 291, 292, 295, 297, or 300 and 246 or 302, respectively;
(xxxviii) 304 and 308, respectively;
(xxxix) 314 and 318, respectively;
(xxxx) 348 and 352, respectively;
(xxxxi) 358 and 362, respectively;
(xxxxii) 368 and 372, respectively;
(xxxxiii) 378 and 382, respectively;
(xxxxiv) 388 and 392, respectively;
(xxxxv) 398 or 432 or 434 or 437 and 402 or 439, respectively, optionally (a) 398 and 402, respectively, (b) 398 and 439, respectively, (c) 432 and 402, respectively, (d) 432 and 439, respectively, (e) 434 and 402, respectively, (f) 434 and 439, respectively, (g) 437 and 402, respectively, or (h) 437 and 439, respectively;
(xxxxvi) 408 or 428 and 412, respectively;
(xxxxvii) 418 and 422, respectively;
(xxxxviii) any one of 408, 446, 448, 458, 459, and 460 and any one of 412, 442, 443, 444, and 445, respectively; or
(xxxxix) 22 and 26, respectively.

19. The antibody or antigen-binding fragment thereof of claim 1, wherein the VH and the VL have at least 85% identity to, or comprise or consist of, the amino acid sequences set forth in SEQ ID NOs.:
(a) 458 and 445, respectively;
(b) 408 and 442, respectively;
(c) 408 and 443, respectively;
(d) 408 and 444, respectively;
(e) 408 and 445, respectively;
(f) 428 and 412, respectively;
(g) 428 and 442, respectively;
(h) 428 and 443, respectively;
(i) 428 and 444, respectively;
(j) 428 and 445, respectively;
(k) 446 and 412, respectively;
(l) 446 and 442, respectively;
(m) 446 and 443, respectively;
(n) 446 and 444, respectively;
(o) 446 and 445, respectively;
(p) 448 and 412, respectively;
(q) 448 and 442, respectively;
(r) 448 and 443, respectively;
(s) 448 and 444, respectively;
(t) 448 and 445, respectively;
(u) 458 and 412, respectively;
(v) 458 and 442, respectively;
(w) 458 and 443, respectively;
(x) 458 and 444, respectively;
(y) 408 and 412, respectively;
(z) 459 and 412, respectively;
(aa) 459 and 442, respectively;
(bb) 459 and 443, respectively;
(cc) 459 and 444, respectively;
(dd) 459 and 445, respectively;
(ee) 460 and 412, respectively;
(ff) 460 and 442, respectively;
(gg) 460 and 443, respectively;
(hh) 460 and 444, respectively; or
(ii) 460 and 445, respectively.

20.-58. (canceled)

59. The antibody or antigen-binding fragment of claim 1, wherein the antibody or antigen-binding fragment further comprises a Fc polypeptide or a fragment thereof.

60. The antibody or antigen-binding fragment of claim 59, wherein the Fc polypeptide or fragment thereof comprises:
(i) a mutation that enhances binding to a FcRn as compared to a reference Fc polypeptide that does not comprise the mutation; and/or
(ii) a mutation that enhances binding to a FcγR as compared to a reference Fc polypeptide that does not comprise the mutation.

61.-75. (canceled)

76. An isolated polynucleotide encoding the antibody or antigen-binding fragment of claim 1, or encoding a VH, a heavy chain, a VL, and/or a light chain of the antibody or the antigen-binding fragment.

77.-79. (canceled)

80. A recombinant vector comprising the polynucleotide of claim 76.

81. A host cell comprising the polynucleotide of claim 76, wherein the polynucleotide is heterologous to the host cell.

82.-95. (canceled)

96. A method of treating a sarbecovirus infection in a subject, the method comprising administering to the subject an effective amount of the antibody or antigen-binding fragment of claim 1.

97.-106. (canceled)

107. A method for in vitro diagnosis of a SARS-CoV-2infection, the method comprising:
(i) contacting a sample from a subject with an antibody or antigen-binding fragment of claim 1; and
(ii) detecting a complex comprising an antigen and the antibody, or comprising an antigen and the antigen-binding fragment.

108.-120. (canceled)
